(19) United States
(12) Patent Application Publication
(10) Pub. No.: US 2023/0373985 A1
(43) Pub. Date: Nov. 23, 2023

(54) KINASE MODULATORS AND METHODS OF USE THEREOF

(71) Applicants: Origenis Gmbh, Martinsried (DE); Neuron23, Inc., San Francisco, CA (US)

(72) Inventors: Roland KOESTLER, Martinsried (DE); Nasser Yehia, Munchen (DE); Jean Tassel, Augsburg (DE); Marcus Wegmann, Munchen (DE); Federico Medda, Munchen (DE); Patricia Perez-Gallan, Munchen (DE); Christiane Gilch, Munchen (DE); Michael Thormann, Planegg (DE); Andreas Treml, Bodenmais (DE); Michael Almstetter, Grasbrunn (DE); Zhonghua Pei, San Francisco, CA (US); Scott Savage, San Francisco, CA (US)

(21) Appl. No.: 18/030,723

(22) PCT Filed: Jul. 12, 2021
(86) PCT No.: PCT/US2021/041270

§ 371 Date: Apr. 6, 2023

Related U.S. Application Data
(60) Provisional application No. 63/137,814, filed on Jan. 15, 2021.
(60) Provisional application No. 63/050,382, filed on Jul. 10, 2020.

(51) Int. Cl. C07D 417/12; C07D 401/14; C07D 491/052; C07D 487/04; C07D 413/12; C07D 249/06; C07D 401/12; C07D 413/14; C07D 417/14; C07D 405/12; C07D 231/38; C07D 498/04

(52) U.S. Cl. CPC C07D 417/12; C07D 401/14; C07D 491/052; C07D 487/04; C07D 413/12; C07D 249/06; C07D 401/12; C07D 413/14; C07D 417/14; C07D 405/12; C07D 231/38; C07D 498/04

(57) ABSTRACT

The present invention relates to novel compounds that modulate the activity of one or more kinases, such as TYK2 or mutants thereof. The compounds, which may be kinase inhibitors or activators, are useful for treating autoimmune diseases, such as, such as psoriasis, lupus, multiple sclerosis, and inflammatory bowel disease.

### CROSS-REFERENCE TO RELATED APPLICATIONS

This application claims the benefit of, and priority to, U.S. Provisional Patent Application No. 63/137,814, filed Jan. 15, 2021, and U.S. Provisional Patent Application No. 63/050,382, filed Jul. 10, 2020, the contents of each of which are incorporated by reference.

### FIELD OF THE INVENTION

The invention provided compounds that modulate the activity of kinases, such as Tyrosine Kinase 2 (TYK2).

### BACKGROUND

A variety of medical conditions that affect millions of people are caused or exacerbated by unregulated activity of protein kinases. For example, aberrant kinase activity is associated with autoimmune diseases, inflammatory diseases, bone diseases, metabolic diseases, neurological and neurodegenerative diseases, cancer, cardiovascular diseases, allergies, asthma, Alzheimer's disease, Parkinson's disease, skin disorders, eye diseases, infectious diseases and hormone-related diseases. For many such disorders, however, no effective inhibitor or activator exists for the particular kinase that causes the disorder or its symptoms. Consequently, patients continue to suffer from an array of disorders due to the lack of suitable medicaments for their conditions.

### SUMMARY

The invention provides compounds that modulate the activity of protein kinases that are associated with human diseases, disorders, and conditions. In particular, compounds of the invention inhibit TYK2, a member of the Janus Kinase (JAK) family of non-receptor protein kinases. Altered or unregulated activity of TYK2 promotes inflammation and is implicated in autoimmune diseases, such as psoriasis, lupus, multiple sclerosis, and inflammatory bowel disease. Thus, embodiments of the invention are useful as pharmaceutical compositions for treatment of such autoimmune conditions. The invention also provides methods of using the compounds to modulate kinase activity in cells and to treat conditions, such as autoimmune conditions, for which modulation of kinase activity provides a therapeutic benefit.

In an aspect, the invention provides compounds of Formula (II):

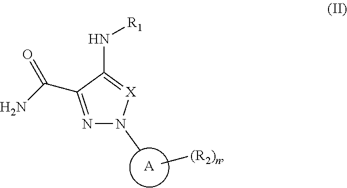

wherein:
X is N or CH;
R₁ is C(O)CH₂R₃, optionally substituted cycloalkyl, optionally substituted cycloheteroalkyl, optionally substituted cycloalkenyl, optionally substituted cycloheteroalkenyl, optionally substituted aryl, or optionally substituted heteroaryl;
each instance of R₂ is independently halo, haloalkyl, cyano, optionally substituted alkoxyl, optionally substituted alkyl, optionally substituted heteroalkyl, optionally substituted alkenyl, optionally substituted heteroalkenyl;
R₃ is optionally substituted cycloalkyl, optionally substituted cycloheteroalkyl, optionally substituted cycloalkenyl, optionally substituted cycloheteroalkenyl, optionally substituted aryl, or optionally substituted heteroaryl;
A is cycloalkyl, cycloheteroalkyl, cycloalkenyl, cycloheteroalkenyl, aryl, or heteroaryl; and
n is 0-5.

The optionally substituted aryl in R₁ may be other than a phenyl.

A may be an aryl substituted with 2 or 3 instances of R₂. A may be a phenyl substituted with 2 or 3 instances of R₂. A may be a heteroaryl substituted with 2 or 3 instances of R₂. A may be a six-membered heteroaryl substituted with 2 or 3 instances of R₂.

The compound of Formula (II) may be represented by Formula (IIa):

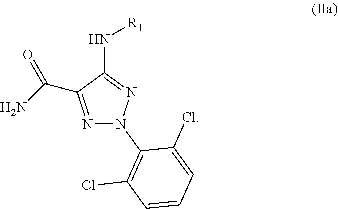

The compound of Formula (IIa) may be represented by one of Formulas (IIa-1), (IIa-2), (IIa-3), (IIa-4), (IIa-5), (IIa-6), (IIa-7), (IIa-8), (IIa-9), (IIa-10), (IIa-11), (IIa-12), (IIa-13), (IIa-14), (IIa-15), (IIa-16), and (IIa-17):

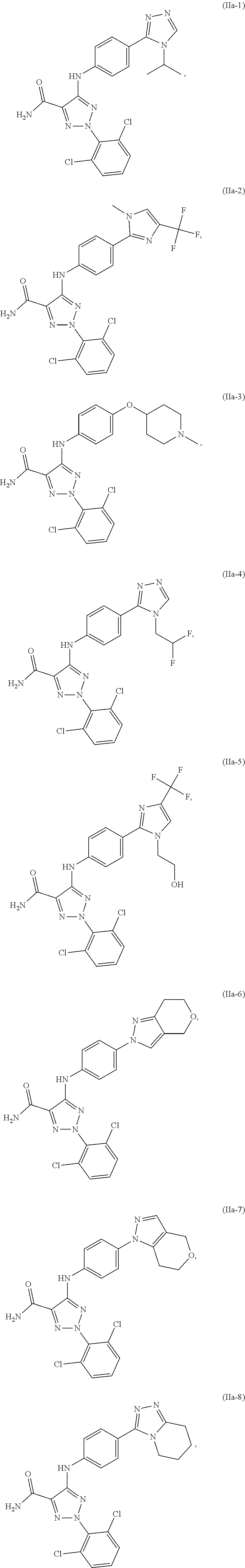

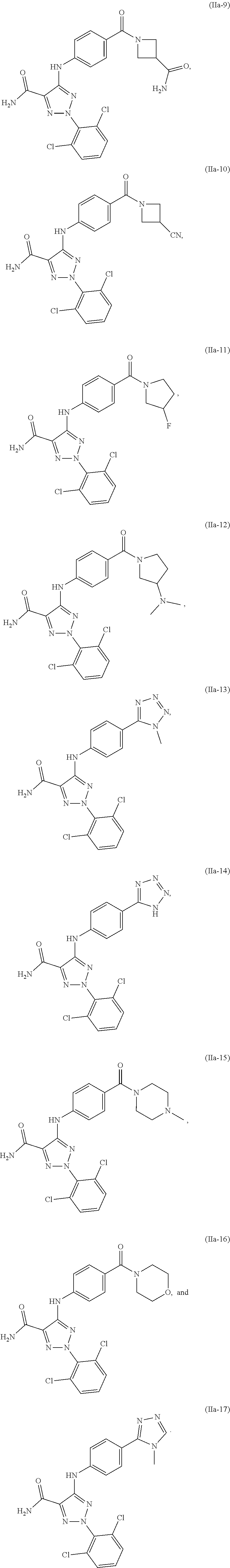

In another aspect, the invention provides compounds of Formula (I):

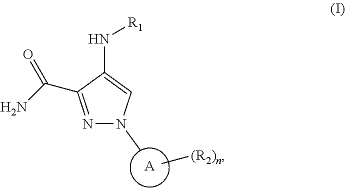

wherein:
R₁ is C(O)CH₂R₃, optionally substituted cycloalkyl, optionally substituted cycloheteroalkyl, optionally substituted cycloalkenyl, optionally substituted cycloheteroalkenyl, optionally substituted aryl, or optionally substituted heteroaryl;
each instance of R₂ is independently halo, haloalkyl, cyano, optionally substituted alkoxyl, optionally substituted alkyl, optionally substituted heteroalkyl, optionally substituted alkenyl, optionally substituted heteroalkenyl;
R₃ is optionally substituted cycloalkyl, optionally substituted cycloheteroalkyl, optionally substituted cycloalkenyl, optionally substituted cycloheteroalkenyl, optionally substituted aryl, or optionally substituted heteroaryl;
A is cycloalkyl, cycloheteroalkyl, cycloalkenyl, cycloheteroalkenyl, aryl, or heteroaryl; and
n is 0-5.

The optionally substituted aryl in R₁ may be other than a phenyl.

A may be an aryl substituted with 2 or 3 instances of R₂. A may be a phenyl substituted with 2 or 3 instances of R₂. A may be a heteroaryl substituted with 2 or 3 instances of R₂. A may be a six-membered heteroaryl substituted with 2 or 3 instances of R₂.

The compound of Formula (I) may be represented by Formula (Ia):

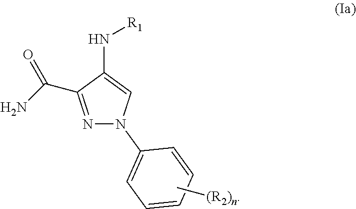

The compound of Formula (Ia) may be represented by one of Formulas (Ia-1), (Ia-2), (Ia-3), (Ia-4), (Ia-5), (Ia-6), and (Ia-7):

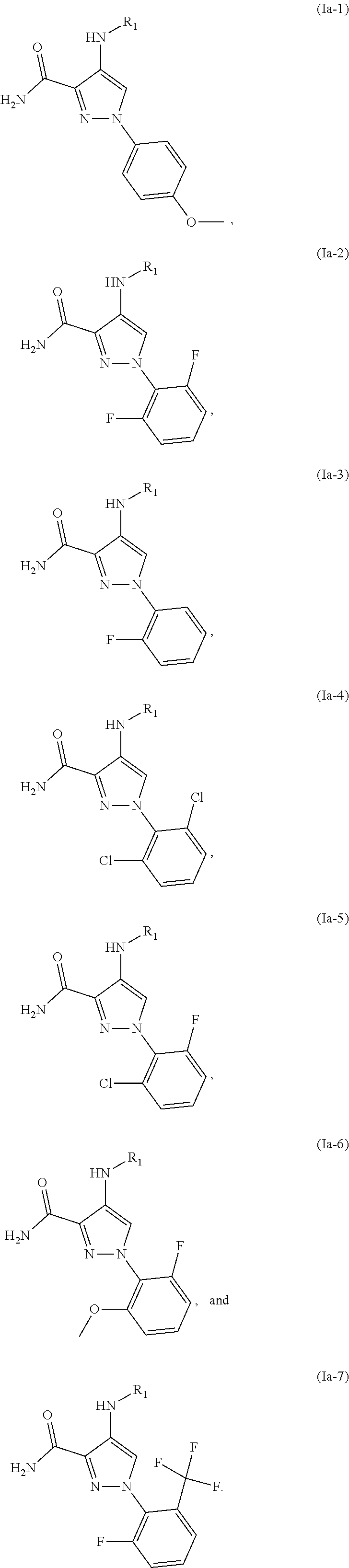

The compound of Formula (I) may be represented by Formula (Ib):

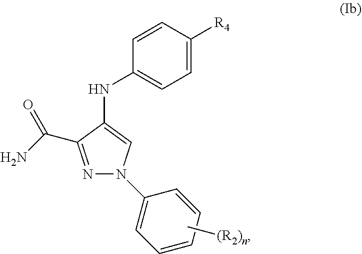

wherein:
R₄ is H, C(O)NR₅R₆, C(O)R₅, S(O)₂R₅, S(O)₂NR₅R₆, P(O)R₇R₈, ORs, optionally substituted cycloalkyl, optionally substituted cycloheteroalkyl, optionally substituted cycloalkenyl, optionally substituted cycloheteroalkenyl, optionally substituted aryl, or optionally substituted heteroaryl;
either:
   R₅ is H, optionally substituted alkyl, optionally substituted heteroalkyl, optionally substituted cycloalkyl, optionally substituted cycloheteroalkyl, optionally substituted cycloalkenyl, optionally substituted cycloheteroalkenyl, optionally substituted aryl, or optionally substituted heteroaryl, and R₆, if present, is H, optionally substituted alkyl, or optionally substituted heteroalkyl; or
R₅ and R₆ together form an optionally substituted cycloalkyl or optionally substituted cycloheteroalkyl;
R₇ is optionally substituted alkyl or optionally substituted heteroalkyl; and
R₈ is optionally substituted alkyl or optionally substituted heteroalkyl.
R₁ may be a heteroaryl having one of the following structures:

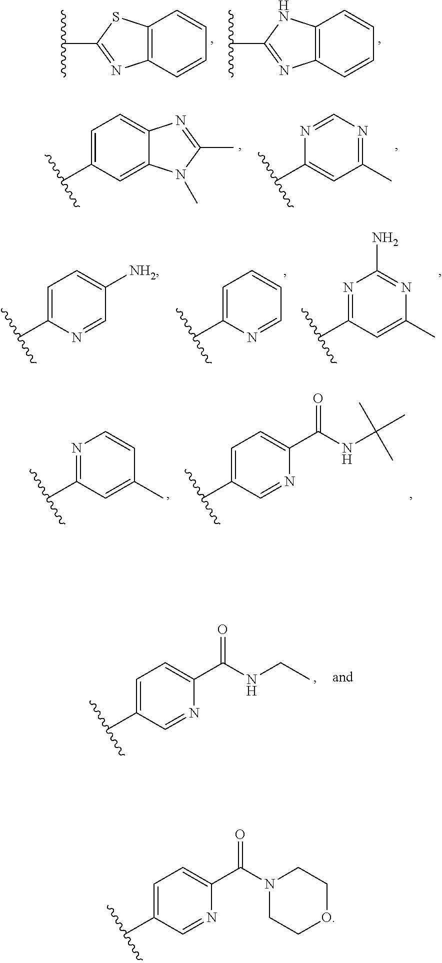

R₃ may have the following structure.

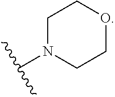 [SMILES: *N1CCOCC1]

In another aspect, the invention provides pharmaceutical compositions containing one or more compounds of the invention, such as any of the compounds described above.

In another aspect, the invention provides methods of modulating the activity of a kinase by contacting cells containing a kinase with one or more compounds of the invention, such as any of those described above.

The compound may inhibit activity of the kinase. The compound may increase activity of the kinase.

The kinase may be a JAK family kinase. The kinase may be, LRRK2, NUAK1, JAK1, JAK2, or TYK2.

In another aspect, the invention provides methods of treating a condition in a subject by administering to the subject a compound of the invention, such as any of those described above.

The condition may be characterized by elevated activity of a kinase. The condition may be characterized by altered activity of a kinase.

The kinase may be a JAK family kinase. The kinase may be LRRK2, NUAK1, JAK1, JAK2, or TYK2.

The condition may be an autoimmune disease, inflammatory disease, bone disease, metabolic disease, neurological or neurodegenerative disease, cancer, cardiovascular disease, allergies, asthma, Alzheimer's disease, Parkinson's disease, skin disorder, eye disease, infectious disease, or hormone-related disease.

In another aspect, the invention provides use of a compound of the invention, such as any of those described above, for making a medicament. In embodiments of the use, the medicament is useful for treating a condition in a subject.

In embodiments of the use the condition is characterize by elevated activity or altered activity of a kinase. In embodiments of the use, the kinase is a JAK family kinase. In embodiments of the use, the kinase is LRRK2, NUAK1, JAK1, JAK2, or TYK2.

In embodiments of the use, the condition is an autoimmune disease, inflammatory disease, bone disease, metabolic disease, neurological or neurodegenerative disease, cancer, cardiovascular disease, allergies, asthma, Alzheimer's disease, Parkinson's disease, skin disorder, eye disease, infectious disease, or hormone-related disease.

### DETAILED DESCRIPTION

### Chemical Definitions

The expression alkyl refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

The expression heteroalkyl refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₁₀ alkyl”). In some embodiments, a heteroalkyl group is a saturated group having 1 to 4 carbon atoms and 1 or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆ alkyl”). The expression alkenyl refers to a radical of a straight-chain or branched hydrocarbon group having from 2 to 20 carbon atoms, one or more carbon-carbon double bonds (e.g., 1, 2, 3, or 4 carbon-carbon double bonds) (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In some embodiments, a heteroalkenyl group has 2 to 9 carbon atoms at least one double bond, and 1, 2, 3, or 4 heteroatoms (“heteroC₂₋₉ alkenyl”). In some embodiments, a heteroalkenyl group has 2 to 8 carbon atoms, at least one double bond, and 1, 2, 3, or 4 heteroatoms (“heteroC₂₋₈ alkenyl”). In some embodiments, a heteroalkenyl group has 2 to 7 carbon atoms, at least one double bond, and 1, 2, 3, or 4 heteroatoms (“heteroC₂₋₇ alkenyl”). In some embodiments, a heteroalkenyl group has 2 to 6 carbon atoms, at least one double bond, and 1, 2, or 3 heteroatoms (“heteroC₂₋₆ alkenyl”). In some embodiments, a heteroalkenyl group has 2 to 5 carbon atoms, at least one double bond, and 1 or 2 heteroatoms (“heteroC₂₋₅ alkenyl”). In some embodiments, a heteroalkenyl group has 2 to 4 carbon atoms, at least one double bond, and 1 or 2 heteroatoms (“heteroC₂₋₄ alkenyl”). In some embodiments, a heteroalkenyl group has 2 to 3 carbon atoms, at least one double bond, and 1 heteroatom (“heteroC₂₋₃ alkenyl”). In some embodiments, a heteroalkenyl group has 2 to 6 carbon atoms, at least one double bond, and 1 or 2 heteroatoms (“heteroCC₂₋₆ alkenyl”).

The expression cycloalkyl refers to a saturated or partially unsaturated (for example, a cycloalkenyl group) cyclic group that contains one or more rings, e.g., 2 or 3 rings, and contains from 3 to 14 ring carbon atoms, such as from 3 to 10 (e.g., 3, 4, 5, 6 or 7) ring carbon atoms. The expression cycloalkyl refers furthermore to groups in which one or more hydrogen atoms have been replaced by fluorine, chlorine, bromine or iodine atoms or by OH, ═O, SH, ═S, NH₂, ═NH, N₃ or NO₂ groups, thus, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetraline, cyclopentylcyclohexyl, fluorocyclohexyl or cyclohex-2-enyl group.

The expression cycloheteroalkyl or heterocycloalkyl refers to a cycloalkyl group as defined above in which one or more (e.g., 1, 2, or 3) ring carbon atoms have been replaced by an oxygen, nitrogen, silicon, selenium, phosphorus or sulfur atom or a SO group or a SO₂ group. A cycloheteroalkyl or heterocycloalkyl group may have 1 or 2 rings containing from 3 to 10 (e.g., 3, 4, 5, 6 or 7) ring atoms (e.g., C, O, N or S). Cycloheteroalkyl or heterocycloalkyl groups include cycloheteroalkenyl or heterocycloalkenyl groups. The expression cycloheteroalkyl or heterocycloalkyl refers furthermore to groups that are substituted by fluorine, chlorine, bromine or iodine atoms or by OH, ═O, SH, ═S, NH₂, ═NH, N₃ or NO₂ groups. Examples are a piperidinyl, prolinyl, imidazolidinyl, piperazinyl, morpholinyl, urotro pinyl, pyrrolidinyl, tetrahydrothiophenyl, tetrahydropyranyl, tetrahydrofuryl or 2-pyrazolinyl group and also lactams, lactones, cyclic imides and cyclic anhydrides.

The expression alkylcycloalkyl refers to groups that contain both cycloalkyl and also alkyl, alkenyl or alkynyl groups in accordance with the above definitions, for example alkylcycloalkyl, cycloalkylalkyl, alkylcycloalkenyl, alkenylcycloalkyl and alkynylcycloalkyl groups. An alkylcycloalkyl group preferably contains a cycloalkyl group that contains one or two rings having from 3 to 10 (e.g., 3, 4, 5, 6 or 7) ring carbon atoms, and one or two alkyl or alkynyl groups having 1 or 2 to 6 carbon atoms.

The expression heteroalkylcycloalkyl refers to alkylcycloalkyl groups as defined above in which one or more (e.g., 1, 2 or 3) carbon atoms have been replaced by an oxygen, nitrogen, silicon, selenium, phosphorus or sulfur atom or a SO group or a SO₂ group. A heteroalkylcycloalkyl group preferably contains 1 or 2 rings having from 3 to 10 (e.g., 3, 4, 5, 6 or 7) ring atoms, and one or two alkyl, alkenyl, alkynyl or heteroalkyl groups having from 1 or 2 to 6 carbon atoms. Examples of such groups are alkylheterocycloalkyl, alkylheterocycloalkenyl, alkenylheterocycloalkyl, alkynylheterocycloalkyl, heteroalkylcycloalkyl, heteroalkylheterocycloalkyl and heteroalkylheterocycloalkenyl, the cyclic groups being saturated or mono-, di- or tri-unsaturated.

The expression aryl refers to an aromatic group that contains one or more rings, e.g., 2 or 3 rings, containing from 6 to 14 ring carbon atoms, such as from 6 to 10 ring carbon atoms. The expression aryl refers furthermore to groups that are substituted by fluorine, chlorine, bromine or iodine atoms or by CH₃, OH, SH, NH₂, N₃ or NO₂ groups. Examples are the phenyl, naphthyl, biphenyl, 2-fluorophenyl, anilinyl, 3-nitrophenyl or 4-hydroxyphenyl group.

The expression heteroaryl refers to an aromatic group that contains one or more rings, e.g., 2 or 3 rings, containing from 5 to 14 ring atoms, such as from 5 to 10 ring atoms, and contains one or more (e.g., 1, 2, 3 or 4) oxygen, nitrogen, phosphorus or sulfur ring atoms. The expression heteroaryl refers furthermore to groups that are substituted by fluorine, chlorine, bromine or iodine atoms or by CH₃, OH, SH, N₃, NH₂ or NO₂ groups. Examples are pyridyl (e.g. 4-pyridyl), imidazolyl (e.g. 2-imidazolyl), phenylpyrrolyl (e.g. 3-phenylpyrrolyl), thiazolyl, isothiazolyl, 1,2,3-triazolyl, 1,2,4-triazolyl, oxadiazolyl, thiadiazolyl, indolyl, indazolyl, tetrazolyl, pyrazinyl, pyrimidinyl, pyridazinyl, oxazolyl, isoxazolyl, triazolyl, tetrazolyl, isoxazolyl, indazolyl, indolyl, benzimidazolyl, benzoxazolyl, benzisoxazolyl, benzthiazolyl, pyridazinyl, quinolinyl, isoquinolinyl, pyrrolyl, purinyl, carbazolyl, acridinyl, pyrimidyl, 2,3′-bifuryl, pyrazolyl (e.g. 3-pyrazolyl) and isoquinolinyl groups.

The expression aralkyl refers to groups containing both aryl and also alkyl, alkenyl, alkynyl and/or cycloalkyl groups in accordance with the above definitions, such as, for example, aryl-alkyl, arylalkenyl, arylalkynyl, arylcycloalkyl, arylcycloalkenyl, alkylarylcycloalkyl and alkylarylcycloalkenyl groups. Specific examples of aralkyls are toluene, xylene, mesitylene, styrene, benzyl chloride, o-fluorotoluene, 1H-indene, tetraline, dihydronaphthalene, indanone, phenylcyclopentyl, cumene, cyclohexylphenyl, fluorene and indane. An aralkyl group preferably contains one or two aromatic ring systems containing from 6 to 10 carbon atoms and one or two alkyl, alkenyl and/or alkynyl groups containing from 1 or 2 to 6 carbon atoms and/or a cycloalkyl group containing 5 or 6 ring carbon atoms.

The expression heteroaralkyl refers to an aralkyl group as defined above in which one or more (e.g., 1, 2, 3 or 4) carbon atoms have been replaced by an oxygen, nitrogen, silicon, selenium, phosphorus, boron or sulfur atom, that is to say to groups containing both aryl or heteroaryl, respectively, and also alkyl, alkenyl, alkynyl and/or heteroalkyl and/or cycloalkyl and/or heterocycloalkyl groups in accordance with the above definitions. A heteroaralkyl group preferably contains one or two aromatic ring systems containing from 5 or 6 to 10 ring carbon atoms and one or two alkyl, alkenyl and/or alkynyl groups containing 1 or 2 to 6 carbon atoms and/or a cycloalkyl group containing 5 or 6 ring carbon atoms, wherein 1, 2, 3 or 4 of these carbon atoms have been replaced by oxygen, sulfur or nitrogen atoms.

Examples are arylheteroalkyl, arylheterocycloalkyl, arylheterocycloalkenyl, arylalkyl heterocycloalkyl, arylalkenylheterocycloalkyl, arylalkynylheterocycloalkyl, arylalkylhetero cycloalkenyl, heteroarylalkyl, heteroarylalkenyl, heteroarylalkynyl, heteroarylheteroalkyl, heteroarylcycloalkyl, heteroarylcycloalkenyl, heteroarylheterocycloalkyl, hetero arylheterocycloalkenyl, heteroarylalkylcycloalkyl, heteroarylalkylheterocycloalkenyl, hetero arylheteroalkylcycloalkyl, heteroarylheteroalkylcycloalkenyl and heteroarylheteroalkylhetero cycloalkyl groups, the cyclic groups being saturated or mono-, di- or tri-unsaturated. Specific examples are a tetrahydroisoquinolinyl, benzoyl, 2- or 3-ethylindolyl, 4-methylpyridino, 2-, 3- or 4-methoxyphenyl, 4-ethoxyphenyl, 2-, 3- or 4-carboxyphenylalkyl group.

As stated above, the expressions cycloalkyl, cycloheteroalkyl, heterocycloalkyl, alkylcycloalkyl, heteroalkylcycloalkyl, aryl, heteroaryl, aralkyl and heteroaralkyl also refer to groups that are substituted by fluorine, chlorine, bromine or iodine atoms or by CH₃, OH, ═O, SH, ═S, NH₂, ═NH, N₃ or NO₂ groups.

The expression carbocyclyl or carbocyclic refers to a radical of a non-aromatic cyclic hydrocarbon group having from 3 to 10 ring carbon atoms (“C₃₋₁₀ carbocyclyl”) and zero heteroatoms in the nonaromatic ring system. In some embodiments, a carbocyclyl group has 3 to 8 ring carbon atoms 10 (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (G), cyclooctenyl (G), bicyclo[2.2.1]heptanyl (C₇), bicyclo[2.2.2]octanyl (G), and the like. Exemplary C₃₋₁₀ carbocyclyl groups include, without 20 limitation, the aforementioned G-s carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

The expression heterocyclyl or heterocyclic refers to a radical of a 3- to 14-membered non-aromatic ring system having ring carbon atoms and 1 to 4 ring heteroatoms, wherein each heteroatom is independently selected from nitrogen, oxygen, sulfur, boron, phosphorus, and silicon (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1,4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups 5 containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

The expression optionally substituted means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Heteroatoms, such as nitrogen, may have substituents, such as any suitable substituent described herein which satisfies the valencies of the heteroatoms and results in the formation of a stable moiety.

For example and without limitation, optional substituents include fluorine, chlorine, bromine, and iodine atoms and CF₃, CN, OH, ═O, SH, ═S, NH₂, ═NH, N₃ and NO₂ groups. Optional substituents also include C₁-C₁₀ alkyl, C₂-C₁₀ alkenyl, C₁-C₁₀ heteroalkyl, C₃-C₁₆ cycloalkyl, C₂-C₁₇ heterocycloalkyl, C₄-C₂₀ alkylcycloalkyl, C₂-C₁₉ heteroalkylcycloalkyl, C₆-C₁₈ aryl, C₁₋₁₇ heteroaryl, C₇-C₂₀ aralkyl or C₂-C₁₉ heteroaralkyl, C₁-C₆ alkyl, C₂-C₆ alkenyl, C₁-C₆ heteroalkyl, C₃-C₁₀ cycloalkyl, C₂-C₉ heterocycloalkyl, C₇-C₁₂ alkylcycloalkyl, C₂-C₁₁ heteroalkylcycloalkyl, C₆-C₁₀ aryl, C₁-C₉ heteroaryl, C₇-C₁₂ aralkyl, C₂-C₁₁ heteroaralkyl, and C₁-C₁₀ haloalkyl groups.

Exemplary substituents are F, Cl, Br, OH, SH, ═O, NH₂, amino, C₁₋₄ alkyl, C₁₋₄ heteroalkyl cyclopropyl, SFs, NO, NO₂.

Other exemplary substituents are F, Cl, Br, OH, SH, ═O, NH₂, C₁₋₄ alkyl (e.g. methyl, ethyl, t-butyl), NMe₂, CONH₂, CH₂NMe₂, NHSO₂Me, C(CH₃)₂CN, COMe, OMe, SMe, COOMe, COOEt, CH₂COOH, OCH₂COOH, COOH, SOMe, SO₂Me, cyclopropyl, SO₂NH₂, SO₂NHMe, SO₂CH₂CH₂OH, NHCH₂CH₂OH, CH₂CH₂OCH₃, SFs, SO₂NMe₂, NO, NO₂, OCF₃, SO₂CF₃, CN or CF₃.

Other exemplary substituents are F, Cl, Br, Me, OMe, CN or CF₃.

The term halogen preferably refers to F, Cl, Br or I.

According to certain embodiments, all alkyl, alkenyl, alkynyl, heteroalkyl, aryl, heteroaryl, cycloalkyl, heterocycloalkyl, alkylcycloalkyl, heteroalkylcycloalkyl, aralkyl and heteroaralkyl groups described herein may optionally be substituted.

When an aryl, heteroaryl, cycloalkyl, alkylcycloalkyl, heteroalkylcycloalkyl, heterocycloalkyl, aralkyl or heteroaralkyl group contains more than one ring, these rings may be bonded to each other via a single or double bond or these rings may be annulated.

Other optional substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{CC}, —C(O)R^{aa}, —CO₂H, —CHO, —C(OR^{cc})₂, —CO₂R^{aa}, —OC(O)R^{aa}, —OCO₂R^{aa}, —C(O)N(R^{bb})₂, —C(O)N(R^{aa})(R^{bb}), —OC(O)N(R^{bb})₂, —NR^{bb}C(O)R^{aa}, —NR^{bb}CO₂R^{aa}, —NR^{bb}C(O)N(R^{bb})₂, —C(NR^{bb})R^{aa}, —C(NR^{bb})OR^{aa}, —OC(NR^{bb})R^{aa}, —OC(NR^{bb})OR^{aa}, —C(NR^{bb})N(R^{bb})₂, —OC(NR^{bb})N(R^{bb})₂, —NR^{bb}C(NR^{bb})N(R^{bb})₂, —C(O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(O)R^{aa}, e.g., —S(O)R^{aa}, —OS(O)R^{aa}, —Si(R^{aa})₃, —OSi(R^{aa})₃—C(S)N(R^{bb})₂, —C(O)SR^{aa}, —C(S)SR^{aa}, —SC(S)SR^{aa}, —SC(O)SR^{aa}, —OC(O)SR^{aa}, —SC(O)OR^{aa}, —SC(O)R^{aa}, —P(O)₂R^{aa}, —OP(O)₂R^{aa}, —P(O)(R^{a})₂, —OP(O)(R^{aa})₂, —OP(O)(OR^{cc})₂, —P(O)₂N(R^{bb})₂, —OP(O)₂N(R^{bb})₂, —P(O)(NR^{bb})₂, —OP(O)(NR^{bb})₂, —NR^{bb}P(O)(OR^{cc})₂, —NR^{bb}P(O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ haloalkyl, C₂₋₁₀ alkenyl, C₃₋₁₀ carbocyclyl, 3-14 membered heterocyclyl, C₆₋₁₄ aryl, and 5-14 membered heteroaryl, wherein each alkyl, alkenyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})₂, ═NNR^{bb}C(O)R^{aa}, ═NNR^{bb}C(O)OR^{aa}, ═NNR^{bb}S(O)₂R^{aa}, ═NR^{bb}, or ═NOR^{cc}; in which:
each instance of R^{aa} is, independently, selected from C₁₋₁₀ alkyl, C₁₋₁₀ heteroalkyl, C₁₋₁₀ haloalkyl, C₂₋₁₀ alkenyl, C₃₋₁₀ cycloalkyl, C₃₋₁₀ cycloheteroalkyl, C₃₋₁₀ cycloalkenyl, C₃₋₁₀ cycloheteroalkenyl, C₃₋₁₀ carbocyclyl, 3-14 membered heterocyclyl, C₆₋₁₄ aryl, and 5-14 membered heteroaryl, or two R^{aa} groups are joined to form a 3-14 membered cycloalkyl, 3-14 membered cycloheteroalkyl, 3-14 membered heterocyclyl or 5-14 membered heteroaryl ring, wherein each alkyl, heteroalkyl, alkenyl, cycloalkyl, cycloheteroalkyl, cycloalkenyl, cycloheteroalkenyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(O)R^{aa}, —C(O)N(R^{cc})₂, —CO₂R^{aa}, —SO₂R^{aa}, —C(NR^{cc})OR^{aa}, —C(NR^{cc})N(R^{cc})₂, —SO₂N(R^{cc})₂, —SO₂R^{cc}, —SO₂OR^{cc}, —SOR^{aa}, —C(S)N(R^{cc})₂, —C(O)SR^{cc}, —C(S)SR^{cc}, —P(O)₂R^{aa}, —P(O)(R^{aa})₂, —P(O)₂N(R^{cc})₂, —P(O)(NR^{cc})₂, C₁₋₁₀ alkyl, C₁₋₁₀ heteroalkyl, C₁₋₁₀ haloalkyl, C₂₋₁₀ alkenyl, C₃₋₁₀ cycloalkyl, C₃₋₁₀ cycloheteroalkyl, C₃₋₁₀ cycloalkenyl, C₃₋₁₀ cycloheteroalkenyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, heteroalkyl, alkenyl, cycloalkyl, cycloheteroalkyl, cycloalkenyl, cycloheteroalkenyl, carbocyclyl, heterocyclyl, aryl, and heteroaryl is independently substituted with 0, 1, 2, 3, 4, or 5 R^{dd} groups;
each instance of RC is, independently, selected from hydrogen, C₁₋₁₀ alkyl, C₁₋₁₀ haloalkyl, C₂₋₁₀ alkenyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(Rn);CX˜, —N(OR^{ee})R^{ff}, —SH, —SR^{ee}, —SSR^{ee}, —C(O)R^{ee}, —CO₂H, —CO₂R^{ee}, —OC(O)R^{ee}, —OCO₂R^{ee}, —C(O)N(R^{ff})₂, —OC(O)N(R^{ff})₂, —NR^{ff}C(O)R^{ee}, —NR^{ff}CO₂R^{ee}, —NR^{ff}C(O)N(R^{ff})₂, —C(NR^{ff})OR^{ee}, —OC(NR^{ff})R^{ee}, —OC(NR^{ee})OR^{ee}, —C(NR^{ff})N(R^{ff})₂, —OC(NR^{ff})N(R^{ff})₂, —NR^{ff}C(NR)N(R^{ff})₂, —NR^{ff}SO₂R^{ee}, —SO₂N(R^{ff})₂, —SO₂R^{ee}, —SO₂OR^{ee}, —OSO₂R^{ee}, —S(O)R^{ee}, e.g., —S(O)R^{cc}, —Si(R^{ee})₃, —OSi(R^{ee})₃, —C(S)N(R^{ff})₂, —C(O)SR^{ee}, —C(S)SR^{ee}, —SC(S)SR^{ee}, —P(O)₂R^{ee}, —P(O)(R^{ee})₂, —OP(O)(R^{ee})₂, —OP(O)(OR^{ee})₂, C₁₋₆ alkyl, C₁₋₆ heteroalkyl, C₁₋₆ haloalkyl, C₂₋₆ alkenyl, C₃₋₁₀ carbocyclyl, 3-10 membered heterocyclyl, C₆₋₁₀ aryl, 5-10 membered heteroaryl, wherein each alkyl, alkenyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ haloalkyl, C₂₋₆ alkenyl, C₃₋₁₀ carbocyclyl, 3-10 membered heterocyclyl, C₆₋₁₀ aryl, 5-10 membered heteroaryl, wherein each alkyl, alkenyl, carbocyclyl, heterocyclyl, aryl, and heteroaryl is independently substituted with 0, 1, 2, 3, 4, or 5 R^{gg} groups;
each instance of R^{f} is, independently, selected from hydrogen, C₁₋₆ alkyl, C₁₋₆ haloalkyl, C₂₋₆ alkenyl, C₃₋₁₀ carbocyclyl, 3-10 membered heterocyclyl, C₆₋₁₀ aryl, 5-10 membered heteroaryl, or two R^{f} groups are joined to form a 3-14 membered heterocyclyl or 5-14 membered heteroaryl ring, wherein each alkyl, alkenyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻-MR⁺X⁻, —N(OC₁₋₆ alkyl)(C₁₋₆ alkyl), —N(OH)(C₁₋₆ alkyl), — NH(OH), —SH, —SC₁₋₆ alkyl, —SS(C₁₋₆ alkyl), —C(O)(C₁₋₆ alkyl), —CO₂H, —CO₂(C₁₋₆ alkyl), —OC(O)(C₁₋₆ alkyl), —OCO₂(C₁₋₆ alkyl), —C(O)NH₂, —C(O)N(C₁₋₆ alkyl)₂, —OC(O)NH(C₁₋₆ alkyl), —NHC(O)(C₁₋₆ alkyl), —N(C₁₋₆ alkyl)C(O)(C₁₋₆ alkyl), —NHCO₂(C₁₋₆ alkyl), —NHC(O)N(C₁₋₆ alkyl)₂, —NHC(O)NH(C₁₋₆ alkyl), —NHC(O)NH₂, —C(NH)O(C₁₋₆ alkyl), —OC(NH)(C₁₋₆ alkyl), —OC(NH)OC₁₋₆ alkyl, —C(NH)N(C₁₋₆ alkyl)₂, —C(NH)NH(C₁₋₆ alkyl), —C(NH)NH₂, —OC(NH)N(C₁₋₆ alkyl)₂, —OC(NH)NH(C₁₋₆ alkyl), —OC(NH)NH₂, —NHC(NH)N(C₁₋₆ alkyl)₂, —NHC(NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, — SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(S)N(C₁₋₆ alkyl)₂, C(S)NH(C₁₋₆ alkyl), C(S)NH₂, —C(O)S(C₁₋₆ alkyl), —C(S)SC₁₋₆ alkyl, —SC(S)SC₁₋₆ alkyl, —P(O)₂(C₁₋₆ alkyl), —P(O)(C₁₋₆ alkyl)₂, —OP(O)(C₁₋₆ alkyl)₂, —OP(O)(OC₁₋₆ alkyl)₂, C₁₋₆ alkyl, C₁₋₆ haloalkyl, C₂₋₆ alkenyl, C₃₋₁₀ carbocyclyl, C₃₋₁₀ aryl, 3-10 membered heterocyclyl, 5-10 membered heteroaryl; or two geminal R^{gg} substituents can be joined to form ═O or ═S; wherein X⁻ is a counterion.

### Compounds

In an aspect, the invention provides compounds of Formula (II):

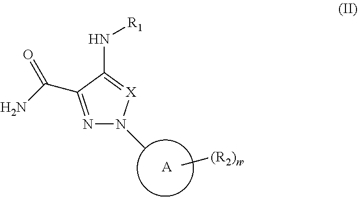

wherein:
X is N or CH;
R₁ is C(O)CH₂R₃, optionally substituted cycloalkyl, optionally substituted cycloheteroalkyl, optionally substituted cycloalkenyl, optionally substituted cycloheteroalkenyl, optionally substituted aryl, or optionally substituted heteroaryl;
each instance of R₂ is independently halo, haloalkyl, cyano, optionally substituted alkoxyl, optionally substituted alkyl, optionally substituted heteroalkyl, optionally substituted alkenyl, optionally substituted heteroalkenyl;
R₃ is optionally substituted cycloalkyl, optionally substituted cycloheteroalkyl, optionally substituted cycloalkenyl, optionally substituted cycloheteroalkenyl, optionally substituted aryl, or optionally substituted heteroaryl;
A is cycloalkyl, cycloheteroalkyl, cycloalkenyl, cycloheteroalkenyl, aryl, or heteroaryl; and
n is 0-5.

The optionally substituted aryl in R₁ may be other than a phenyl.

A may be an aryl substituted with 2 or 3 instances of R₂. A may be a phenyl substituted with 2 or 3 instances of R₂. A may be a heteroaryl substituted with 2 or 3 instances of R₂. A may be a six-membered heteroaryl substituted with 2 or 3 instances of R₂.

The compound of Formula (II) may be represented by Formula (IIa):

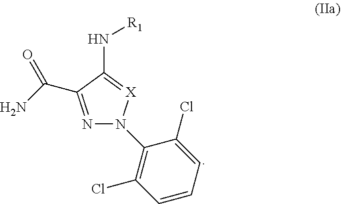

The compound of Formula (IIa) may be represented by one of Formulas (IIa-1), (IIa-2), (IIa-3), (IIa-4), (IIa-5), (IIa-6), (IIa-7), (IIa-8), (IIa-9), (IIa-10), (IIa-11), (IIa-12), (IIa-13), (IIa-14), (IIa-15), (IIa-16), and (IIa-17):

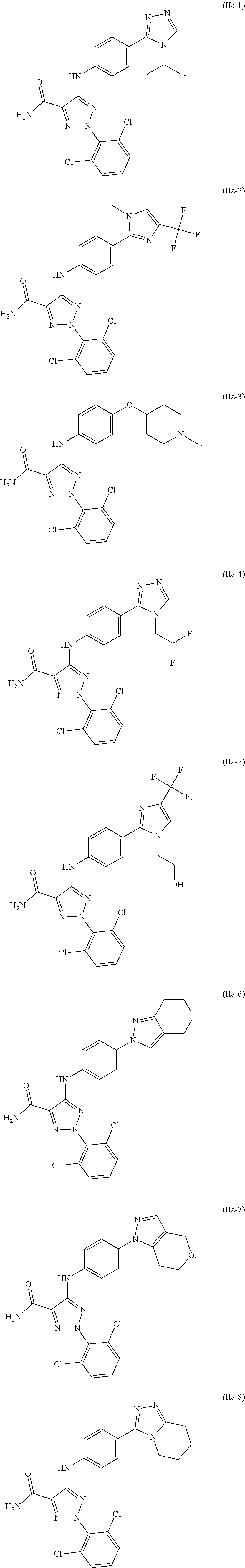

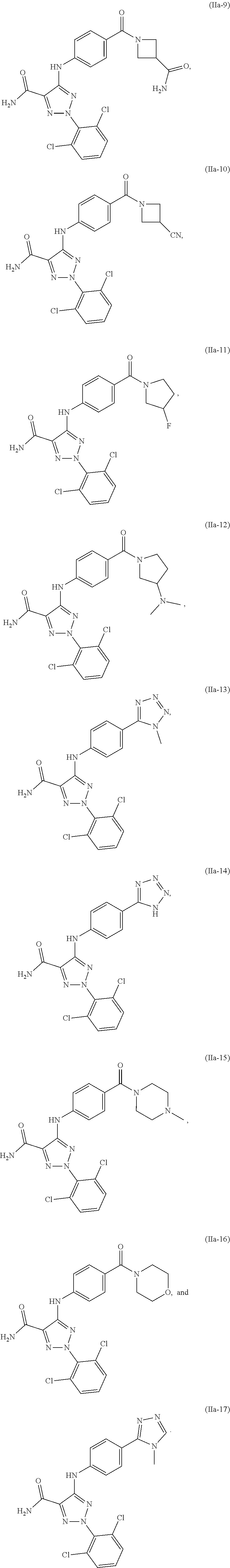

The compound of Formula (11) may have one of the structures shown in Table 1a.

TABLE 1a

| Compound | | |
|---|---|---|
| No. | Structure | IUPAC-Name |
| 101 | 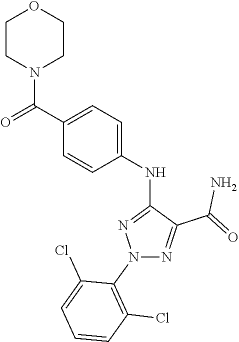 [SMILES: NC(=O)c1nn(-c2c(Cl)cccc2Cl)nc1Nc1ccc(C(=O)N2CCOCC2)cc1] | 2-(2,6-Dichloro-phenyl)-5-[4- (morpholine-4-carbonyl)- phenylamino]-2H-[1,2,3]triazole-4- carboxylic acid amide |
| 102 | 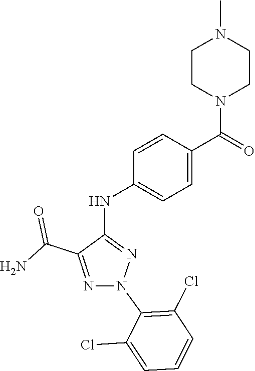 [SMILES: CN1CCN(C(=O)c2ccc(Nc3nn(-c4c(Cl)cccc4Cl)nc3C(N)=O)cc2)CC1] | 2-(2,6-Dichloro-phenyl)-5-[4-(4- methyl-piperazine-1-carbonyl)- phenylamino]-2H-[1,2,3]triazole-4- carboxylic acid amide |
| 103 | 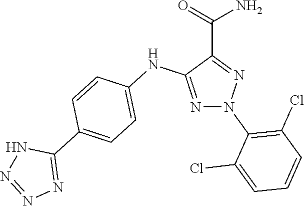 [SMILES: NC(=O)c1nn(-c2c(Cl)cccc2Cl)nc1Nc1ccc(-c2nnn[nH]2)cc1] | 2-(2,6-Dichloro-phenyl)-5-[4-(1H- tetrazol-5-yl)-phenylamino]-2H- [1,2,3]triazole-4-carboxylic acid amide |
| 104 | 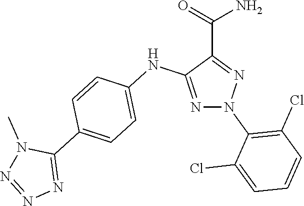 [SMILES: Cn1nnnc1-c1ccc(Nc2nn(-c3c(Cl)cccc3Cl)nc2C(N)=O)cc1] | 2-(2,6-Dichloro-phenyl)-5-[4-(1- methyl-1H-tetrazol-5-yl)- phenylamino]-2H-[1,2,3]triazole-4- carboxylic acid |
| 105 | 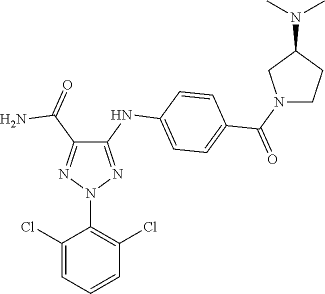 [SMILES: CN(C)[C@H]1CCN(C(=O)c2ccc(Nc3nn(-c4c(Cl)cccc4Cl)nc3C(N)=O)cc2)C1] | 2-(2,6-Dichloro-phenyl)-5-[4-((S)-3- dimethylamino-pyrrolidine-1- carbonyl)-phenylamino]-2H- [1,2,3]triazole-4-carboxylic acid amide |
| 106 | 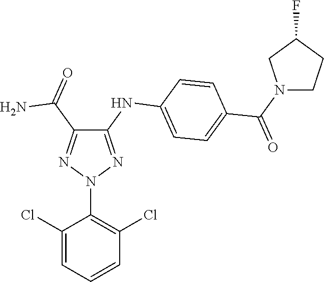 [SMILES: NC(=O)c1nn(-c2c(Cl)cccc2Cl)nc1Nc1ccc(C(=O)N2CC[C@@H](F)C2)cc1] | 2-(2,6-Dichloro-phenyl)-5-[4-((R)-3- fluoro-pyrrolidine-1-carbonyl)- phenylamino]-2H-[1,2,3]triazole-4- carboxylic acid amide |
| 107 | 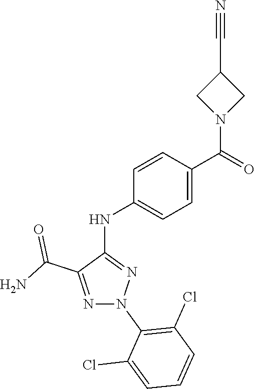 [SMILES: N#CC1CN(C(=O)c2ccc(Nc3nn(-c4c(Cl)cccc4Cl)nc3C(N)=O)cc2)C1] | 5-[4-(3-Cyano-azetidine-1-carbonyl)- phenylamino]-2-(2,6-dichloro- phenyl)-2H-[1,2,3]triazole-4- carboxylic acid amide |
| 108 | 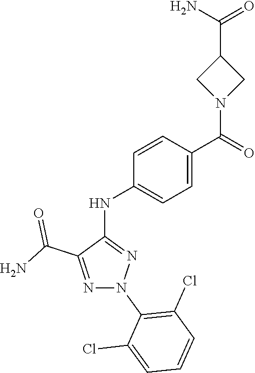 [SMILES: NC(=O)c1nn(-c2c(Cl)cccc2Cl)nc1Nc1ccc(C(=O)N2CC(C(N)=O)C2)cc1] | 2-(2,6-dichlorophenyl)-5-((4-(1-(2- hydroxyethyl)-4-(trifluoromethyl)-1H- imidazol-2-yl)phenyl)amino)-2H- 1,2,3-triazole-4-carboxamide |
| 109 | 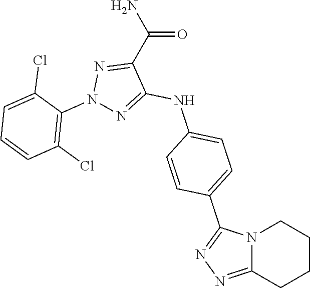 [SMILES: NC(=O)c1nn(-c2c(Cl)cccc2Cl)nc1Nc1ccc(-c2nnc3n2CCCC3)cc1] | 2-(2,6-Dichloro-phenyl)-5-[4-(5,6,7,8- tetrahydro-[1,2,4]triazolo[4,3- a]pyridin-3-yl)-phenylamino]-2H- [1,2,3]triazole-4-carboxylic acid amide |
| 110 | 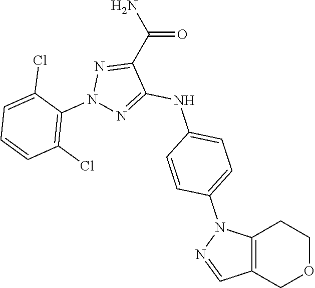 [SMILES: NC(=O)c1nn(-c2c(Cl)cccc2Cl)nc1Nc1ccc(-n2ncc3c2CCOC3)cc1] | 2-(2,6-Dichloro-phenyl)-5-[4-(6,7- dihydro-4H-pyrano[4,3-c]pyrazol-1- yl)-phenylamino]-2H-[1,2,3]triazole- 4-carboxylic acid amide |
| 111 | 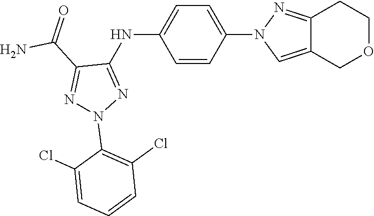 [SMILES: NC(=O)c1nn(-c2c(Cl)cccc2Cl)nc1Nc1ccc(-n2cc3c(n2)CCOC3)cc1] | 2-(2,6-Dichloro-phenyl)-5-[4-(6,7- dihydro-4H-pyrano[4,3-c]pyrazol-2- yl)-phenylamino]-2H-[1,2,3]triazole- 4-carboxylic acid amide |
| 112 | 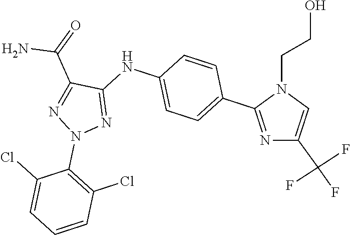 [SMILES: NC(=O)c1nn(-c2c(Cl)cccc2Cl)nc1Nc1ccc(-c2nc(C(F)(F)F)cn2CCO)cc1] | 2-(2,6-dichlorophenyl)-5-((4-(1-(2- hydroxyethyl)-4-(trifluoromethyl)-1H- imidazol-2-yl)phenyl)amino)-2H- 1,2,3-triazole-4-carboxamide |
| 113 | 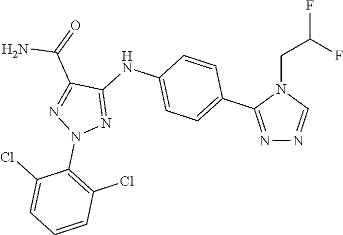 [SMILES: NC(=O)c1nn(-c2c(Cl)cccc2Cl)nc1Nc1ccc(-c2nncn2CC(F)F)cc1] | 2-(2,6-dichlorophenyl)-5-((4-(4-(2,2- difluoroethyl)-4H-1,2,4-triazol-3- yl)phenyl)amino)-2H-1,2,3-triazole-4- carboxamide |
| 114 | 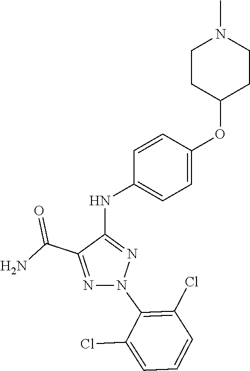 [SMILES: CN1CCC(Oc2ccc(Nc3nn(-c4c(Cl)cccc4Cl)nc3C(N)=O)cc2)CC1] | 2-(2,6-dichlorophenyl)-5-((4-((1- methylpiperidin-4- yl)oxy)phenyl)amino)-2H-1,2,3- triazole-4-carboxamide |
| 115 | 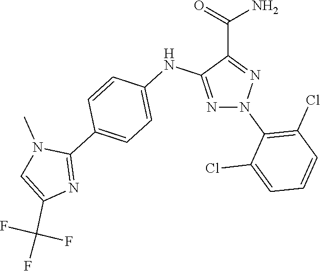 [SMILES: Cn1cc(C(F)(F)F)nc1-c1ccc(Nc2nn(-c3c(Cl)cccc3Cl)nc2C(N)=O)cc1] | 2-(2,6-dichlorophenyl)-5-((4-(1- methyl-4-(trifluoromethyl)-1H- imidazol-2-yl)phenyl)amino)-2H- 1,2,3-triazole-4-carboxamide |
| 116 | 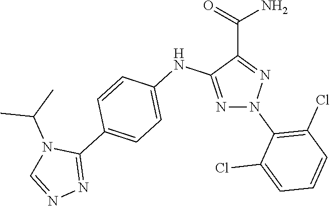 [SMILES: CC(C)n1cnnc1-c1ccc(Nc2nn(-c3c(Cl)cccc3Cl)nc2C(N)=O)cc1] | 2-(2,6-dichlorophenyl)-5-((4-(4- isopropyl-4H-1,2,4-triazol-3- yl)phenyl)amino)-2H-1,2,3-triazole-4- carboxamide |
| 117 | 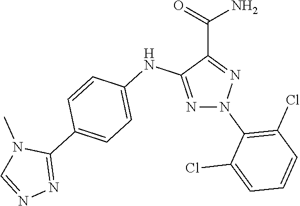 [SMILES: Cn1cnnc1-c1ccc(Nc2nn(-c3c(Cl)cccc3Cl)nc2C(N)=O)cc1] | 2-(2,6-dichlorophenyl)-5-((4-(4- methyl-4H-1,2,4-triazol-3- yl)phenyl)amino)-2H-1,2,3-triazole-4- carboxamide |
| 118 | 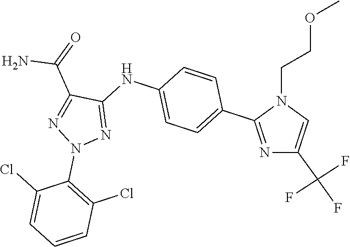 [SMILES: COCCn1cc(C(F)(F)F)nc1-c1ccc(Nc2nn(-c3c(Cl)cccc3Cl)nc2C(N)=O)cc1] | 2-(2,6-dichlorophenyl)-5-((4-(1-(2- methoxyethyl)-4-(trifluoromethyl)- 1H-imidazol-2-yl)phenyl)amino)-2H- 1,2,3-triazole-4-carboxamide |
| 119 | 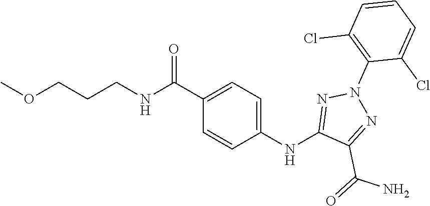 [SMILES: COCCCNC(=O)c1ccc(Nc2nn(-c3c(Cl)cccc3Cl)nc2C(N)=O)cc1] | 2-(2,6-dichlorophenyl)-5-((4-((3- methoxypropyl)carbamoyl)phenyl) amino)-2H-1,2,3-triazole-4-carboxamide |
| 120 | 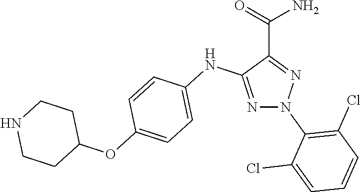 [SMILES: NC(=O)c1nn(-c2c(Cl)cccc2Cl)nc1Nc1ccc(OC2CCNCC2)cc1] | 2-(2,6-dichlorophenyl)-5-((4- (piperidin-4-yloxy)phenyl)amino)-2H- 1,2,3-triazole-4-carboxamide |
| 121 | 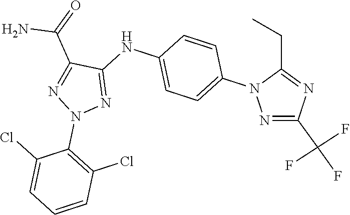 [SMILES: CCc1nc(C(F)(F)F)nn1-c1ccc(Nc2nn(-c3c(Cl)cccc3Cl)nc2C(N)=O)cc1] | 2-(2,6-dichlorophenyl)-5-((4-(5-ethyl- 3-(trifluoromethyl)-1H-1,2,3-triazol- 1-yl)phenyl)amino)-2H-1,2,3-triazole- 4-carboxamide |
| 122 | 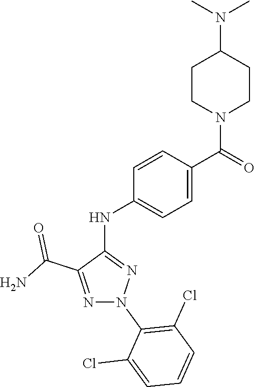 [SMILES: CN(C)C1CCN(C(=O)c2ccc(Nc3nn(-c4c(Cl)cccc4Cl)nc3C(N)=O)cc2)CC1] | 2-(2,6-dichlorophenyl)-5-((4-(4- (dimethylamino)piperidine-1- carbonyl)phenyl)amino)-2H-1,2,3- triazole-4-carboxamide |
| 123 | 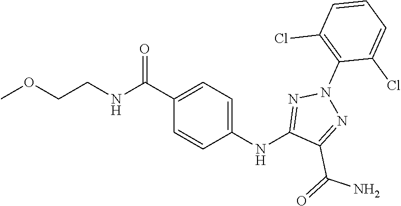 [SMILES: COCCNC(=O)c1ccc(Nc2nn(-c3c(Cl)cccc3Cl)nc2C(N)=O)cc1] | 2-(2,6-dichlorophenyl)-5-((4-((2- methoxyethyl)carbamoyl)phenyl)amino)- 2H-1,2,3-triazole-4-carboxamide |
| 124 | 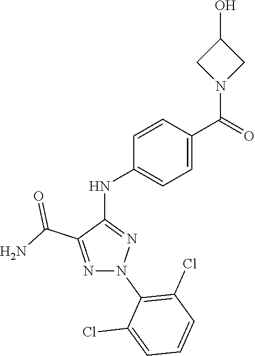 [SMILES: NC(=O)c1nn(-c2c(Cl)cccc2Cl)nc1Nc1ccc(C(=O)N2CC(O)C2)cc1] | 2-(2,6-dichlorophenyl)-5-((4-(3- hydroxyazetidine-1- carbonyl)phenyl)amino)-2H-1,2,3- triazole-4-carboxamide |
| 125 | 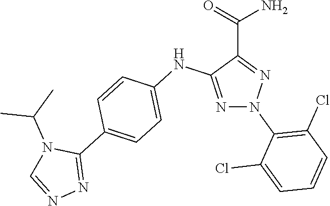 [SMILES: CC(C)n1cnnc1-c1ccc(Nc2nn(-c3c(Cl)cccc3Cl)nc2C(N)=O)cc1] | 2-(2,6-dichlorophenyl)-5-((4-(4- isopropyl-4H-1,2,4-triazol-3- yl)phenyl)amino)-2H-1,2,3-triazole-4- carboxamide |
| 126 | 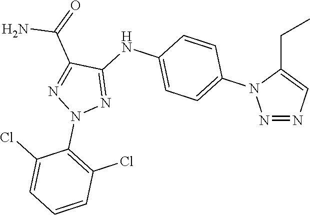 [SMILES: CCc1cnnn1-c1ccc(Nc2nn(-c3c(Cl)cccc3Cl)nc2C(N)=O)cc1] | 2-(2,6-dichlorophenyl)-5-((4-(5-ethyl- 1H-1,2,3-triazol-1-yl)phenyl)amino)- 2H-1,2,3-triazole-4-carboxamide |
| 127 | 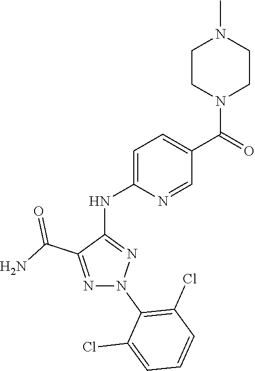 [SMILES: CN1CCN(C(=O)c2ccc(Nc3nn(-c4c(Cl)cccc4Cl)nc3C(N)=O)nc2)CC1] | 2-(2,6-dichlorophenyl)-5-((5-(4- methylpiperazine-1-carbonyl)pyridin- 2-yl)amino)-2H-1,2,3-triazole-4- carboxamide |
| 128 | 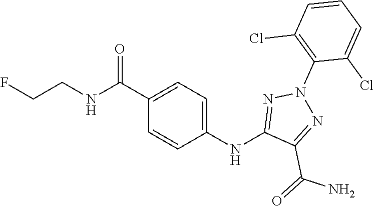 [SMILES: NC(=O)c1nn(-c2c(Cl)cccc2Cl)nc1Nc1ccc(C(=O)NCCF)cc1] | 2-(2,6-dichlorophenyl)-5-((4-((2- fluoroethyl)carbamoyl)phenyl)amino)- 2H-1,2,3-triazole-4-carboxamide |
| 129 | 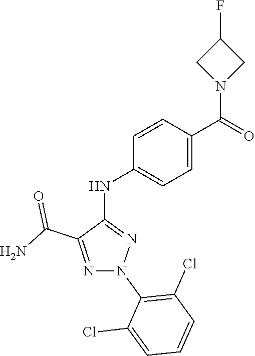 [SMILES: NC(=O)c1nn(-c2c(Cl)cccc2Cl)nc1Nc1ccc(C(=O)N2CC(F)C2)cc1] | 2-(2,6-dichlorophenyl)-5-((4-(3- fluoroazetidine-1- carbonyl)phenyl)amino)-2H-1,2,3- triazole-4-carboxamide |
| 130 | 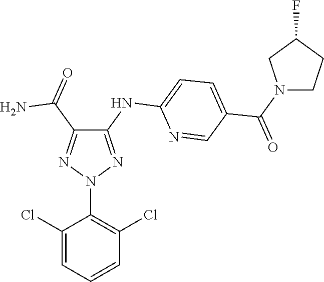 [SMILES: NC(=O)c1nn(-c2c(Cl)cccc2Cl)nc1Nc1ccc(C(=O)N2CC[C@@H](F)C2)cn1] | (S)-2-(2,6-dichlorophenyl)-5-((5-(3- fluoropyrrolidine-1-carbonyl)pyridin- 2-yl)amino)-2H-1,2,3-triazole-4- carboxamide |
| 131 | 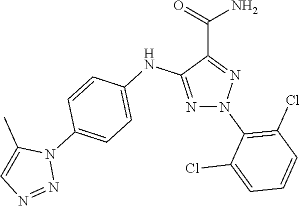 [SMILES: Cc1cnnn1-c1ccc(Nc2nn(-c3c(Cl)cccc3Cl)nc2C(N)=O)cc1] | 2-(2,6-dichlorophenyl)-5-((4-(5- methyl-1H-1,2,3-triazol-1- yl)phenyl)amino)-2H-1,2,3-triazole-4- carboxamide |
| 132 | 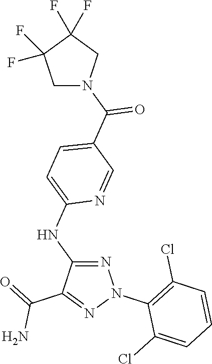 [SMILES: NC(=O)c1nn(-c2c(Cl)cccc2Cl)nc1Nc1ccc(C(=O)N2CC(F)(F)C(F)(F)C2)cn1] | 2-(2,6-dichlorophenyl)-5-((5-(3,3,4,4- tetrafluoropyrrolidine-1- carbonyl)pyridin-2-yl)amino)-2H- 1,2,3-triazole-4-carboxamide |
| 133 | 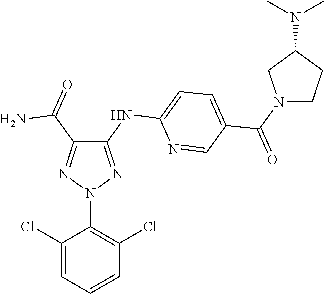 [SMILES: CN(C)[C@@H]1CCN(C(=O)c2ccc(Nc3nn(-c4c(Cl)cccc4Cl)nc3C(N)=O)nc2)C1] | (R)-2-(2,6-dichlorophenyl)-5-((5-(3- (dimethylamino)pyrrolidine-1- carbonyl)pyridin-2-yl)amino)-2H- 1,2,3-triazole-4-carboxamide |
| 134 | 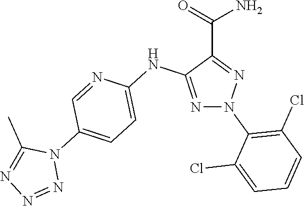 [SMILES: Cc1nnnn1-c1ccc(Nc2nn(-c3c(Cl)cccc3Cl)nc2C(N)=O)nc1] | 2-(2,6-dichlorophenyl)-5-((5-(5- methyl-1H-tetrazol-1-yl)pyridin-2- yl)amino)-2H-1,2,3-triazole-4- carboxamide |
| 135 | 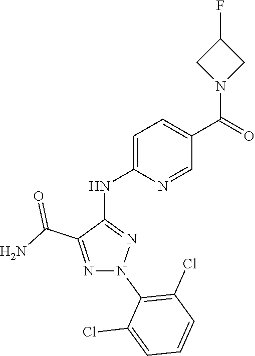 [SMILES: NC(=O)c1nn(-c2c(Cl)cccc2Cl)nc1Nc1ccc(C(=O)N2CC(F)C2)cn1] | 2-(2,6-dichlorophenyl)-5-((5-(3- fluoroazetidine-1-carbonyl)pyridin-2- yl)amino)-2H-1,2,3-triazole-4- carboxamide |
| 136 | 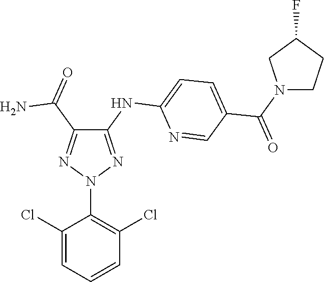 [SMILES: NC(=O)c1nn(-c2c(Cl)cccc2Cl)nc1Nc1ccc(C(=O)N2CC[C@@H](F)C2)cn1] | (R)-2-(2,6-dichlorophenyl)-5-((5-(3- fluoropyrrolidine-1-carbonyl)pyridin- 2-yl)amino)-2H-1,2,3-triazole-4- carboxamide |
| 137 | 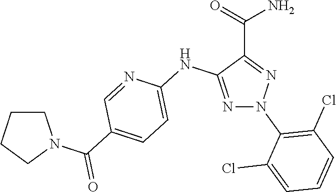 [SMILES: NC(=O)c1nn(-c2c(Cl)cccc2Cl)nc1Nc1ccc(C(=O)N2CCCC2)cn1] | 2-(2,6-dichlorophenyl)-5-((5- (pyrrolidine-1-carbonyl)pyridin-2- yl)amino)-2H-1,2,3-triazole-4- carboxamide |
| 138 | 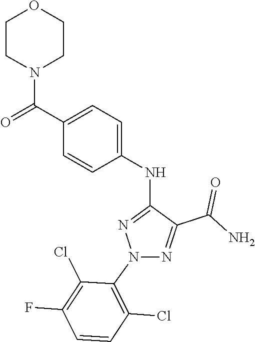 [SMILES: NC(=O)c1nn(-c2c(Cl)ccc(F)c2Cl)nc1Nc1ccc(C(=O)N2CCOCC2)cc1] | 2-(2,6-dichloro-3-fluorophenyl)-5-((4- (morpholine-4- carbonyl)phenyl)amino)-2H-1,2,3- triazole-4-carboxamide |
| 139 | 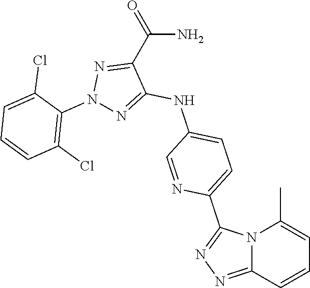 [SMILES: Cc1cccc2nnc(-c3ccc(Nc4nn(-c5c(Cl)cccc5Cl)nc4C(N)=O)cn3)n12] | 2-(2,6-dichlorophenyl)-5-((6-(5- methyl-[1,2,4]triazolo[4,3-a]pyridin- 3-yl)pyridin-3-yl)amino)-2H-1,2,3- triazole-4-carboxamide |
| 140 | 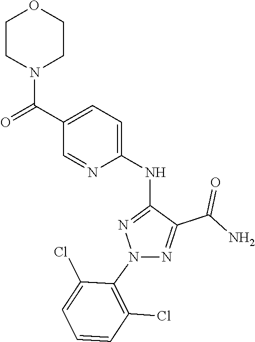 [SMILES: NC(=O)c1nn(-c2c(Cl)cccc2Cl)nc1Nc1ccc(C(=O)N2CCOCC2)cn1] | 2-(2,6-dichlorophenyl)-5-((5- (morpholine-4-carbonyl)pyridin-2- yl)amino)-2H-1,2,3-triazole-4- carboxamide |
| 141 | 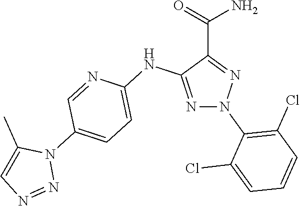 [SMILES: Cc1cnnn1-c1ccc(Nc2nn(-c3c(Cl)cccc3Cl)nc2C(N)=O)nc1] | 2-(2,6-dichlorophenyl)-5-((5-(5- methyl-1H-1,2,3-triazol-1-yl)pyridin- 2-yl)amino)-2H-1,2,3-triazole-4- carboxamide |
| 142 | 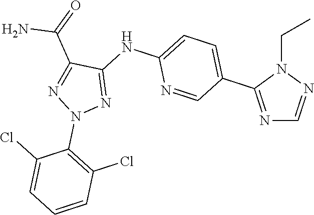 [SMILES: CCn1ncnc1-c1ccc(Nc2nn(-c3c(Cl)cccc3Cl)nc2C(N)=O)nc1] | 2-(2,6-dichlorophenyl)-5-((5-(1-ethyl- 1H-1,2,4-triazol-5-yl)pyridin-2- yl)amino)-2H-1,2,3-triazole-4- carboxamide |
| 143 | 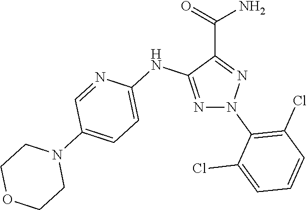 [SMILES: NC(=O)c1nn(-c2c(Cl)cccc2Cl)nc1Nc1ccc(N2CCOCC2)cn1] | 2-(2,6-dichlorophenyl)-5-((5- morpholinopyridin-2-yl)amino)-2H- 1,2,3-triazole-4-carboxamide |
| 144 | 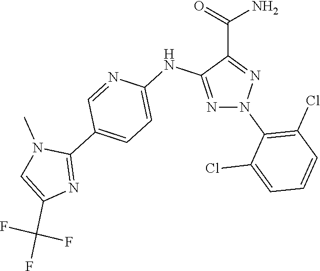 [SMILES: Cn1cc(C(F)(F)F)nc1-c1ccc(Nc2nn(-c3c(Cl)cccc3Cl)nc2C(N)=O)nc1] | 2-(2,6-dichlorophenyl)-5-((5-(1- methyl-4-(trifluoromethyl)-1H- imidazol-2-yl)pyridin-2-yl)amino)- 2H-1,2,3-triazole-4-carboxamide |
| 145 | 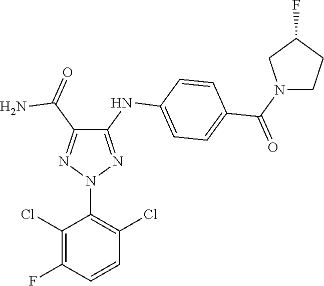 [SMILES: NC(=O)c1nn(-c2c(Cl)ccc(F)c2Cl)nc1Nc1ccc(C(=O)N2CC[C@@H](F)C2)cc1] | (R)-2-(2,6-dichloro-3-fluorophenyl)- 5-((4-(3-fluoropyrrolidine-1- carbonyl)phenyl)amino)-2H-1,2,3- triazole-4-carboxamide |
| 146 | 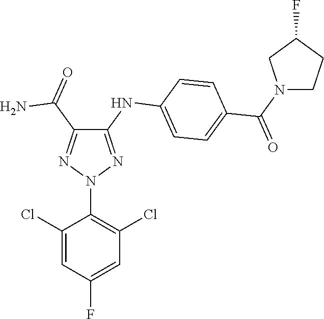 [SMILES: NC(=O)c1nn(-c2c(Cl)cc(F)cc2Cl)nc1Nc1ccc(C(=O)N2CC[C@@H](F)C2)cc1] | (R)-2-(2,6-dichloro-4-fluorophenyl)- 5-((4-(3-fluoropyrrolidine-1- carbonyl)phenyl)amino)-2H-1,2,3- triazole-4-carboxamide |
| 147 | 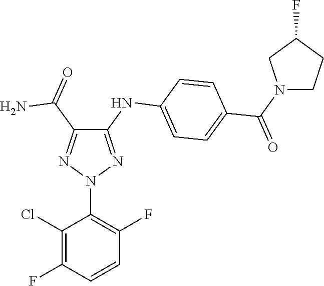 [SMILES: NC(=O)c1nn(-c2c(F)ccc(F)c2Cl)nc1Nc1ccc(C(=O)N2CC[C@@H](F)C2)cc1] | (R)-2-(2-chloro-3,6-difluorophenyl)- 5-((4-(3-fluoropyrrolidine-1- carbonyl)phenyl)amino)-2H-1,2,3- triazole-4-carboxamide |
| 148 | 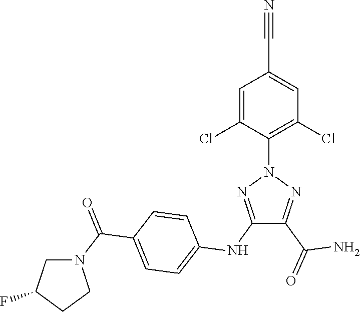 [SMILES: N#Cc1cc(Cl)c(-n2nc(Nc3ccc(C(=O)N4CC[C@H](F)C4)cc3)c(C(N)=O)n2)c(Cl)c1] | (R)-2-(2,6-dichloro-4-cyanophenyl)-5- ((4-(3-fluoropyrrolidine-1- carbonyl)phenyl)amino)-2H-1,2,3- triazole-4-carboxamide |
| 149 | 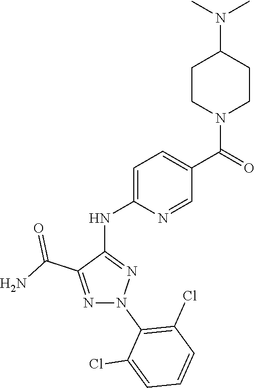 [SMILES: CN(C)C1CCN(C(=O)c2ccc(Nc3nn(-c4c(Cl)cccc4Cl)nc3C(N)=O)nc2)CC1] | 2-(2,6-dichlorophenyl)-5-((5-(4- (dimethylamino)piperidine-1- carbonyl)pyridin-2-yl)amino)-2H- 1,2,3-triazole-4-carboxamide |
| 150 | 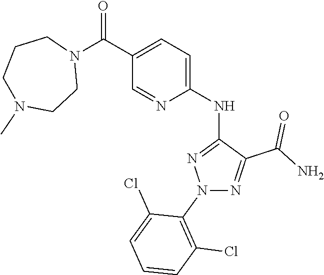 [SMILES: CN1CCCN(C(=O)c2ccc(Nc3nn(-c4c(Cl)cccc4Cl)nc3C(N)=O)nc2)CC1] | 2-(2,6-dichlorophenyl)-5-((5-(4- methyl-1,4-diazepane-1- carbonyl)pyridin-2-yl)amino)-2H- 1,2,3-triazole-4-carboxamide |
| 151 | 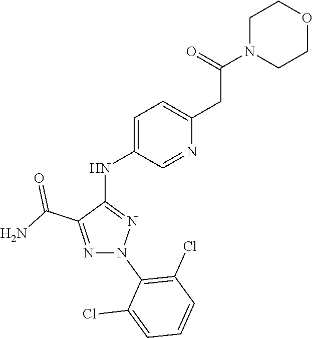 [SMILES: NC(=O)c1nn(-c2c(Cl)cccc2Cl)nc1Nc1ccc(CC(=O)N2CCOCC2)nc1] | 2-(2,6-dichlorophenyl)-5-((6-(2- morpholino-2-oxoethyl)pyridin-3- yl)amino)-2H-1,2,3-triazole-4- carboxamide |
| 152 | 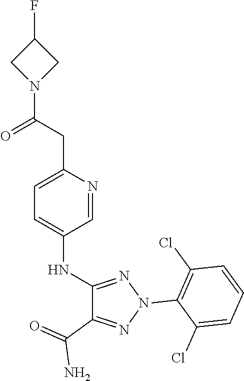 [SMILES: NC(=O)c1nn(-c2c(Cl)cccc2Cl)nc1Nc1ccc(CC(=O)N2CC(F)C2)nc1] | 2-(2,6-dichlorophenyl)-5-((6-(2-(3- fluoroazetidin-1-yl)-2- oxoethyl)pyridin-3-yl)amino)-2H- 1,2,3-triazole-4-carboxamide |
| 153 | 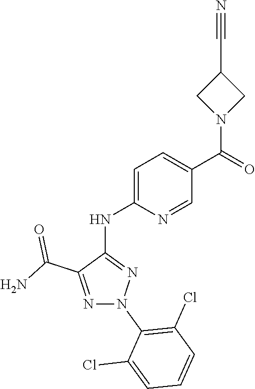 [SMILES: N#CC1CN(C(=O)c2ccc(Nc3nn(-c4c(Cl)cccc4Cl)nc3C(N)=O)nc2)C1] | 5-((5-(3-cyanoazetidine-1- carbonyl)pyridin-2-yl)amino)-2-(2,6- dichlorophenyl)-2H-1,2,3-triazole-4- carboxamide |
| 154 | 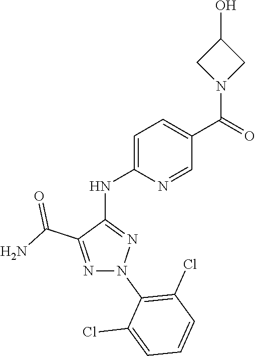 [SMILES: NC(=O)c1nn(-c2c(Cl)cccc2Cl)nc1Nc1ccc(C(=O)N2CC(O)C2)cn1] | 2-(2,6-dichlorophenyl)-5-((5-(3- hydroxyazetidine-1-carbonyl)pyridin- 2-yl)amino)-2H-1,2,3-triazole-4- carboxamide |
| 155 | 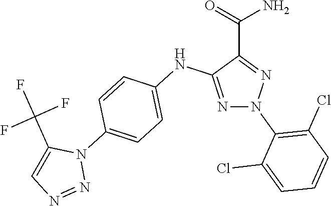 [SMILES: NC(=O)c1nn(-c2c(Cl)cccc2Cl)nc1Nc1ccc(-n2nncc2C(F)(F)F)cc1] | 2-(2,6-dichlorophenyl)-5-((4-(5- (trifluoromethyl)-1H-1,2,3-triazol-1- yl)phenyl)amino)-2H-1,2,3-triazole-4- carboxamide |
| 156 | 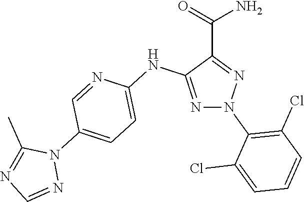 [SMILES: Cc1ncnn1-c1ccc(Nc2nn(-c3c(Cl)cccc3Cl)nc2C(N)=O)nc1] | 2-(2,6-dichlorophenyl)-5-((5-(5- methyl-1H-1,2,4-triazol-1-yl)pyridin- 2-yl)amino)-2H-1,2,3-triazole-4- carboxamide |
| 157 | 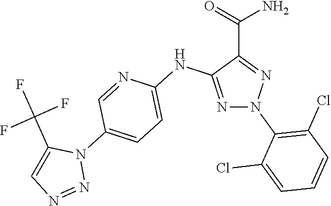 [SMILES: NC(=O)c1nn(-c2c(Cl)cccc2Cl)nc1Nc1ccc(-n2nncc2C(F)(F)F)cn1] | 2-(2,6-dichlorophenyl)-5-((5-(5- (trifluoromethyl)-1H-1,2,3-triazol-1- yl)pyridin-2-yl)amino)-2H-1,2,3- triazole-4-carboxamide |
| 158 | 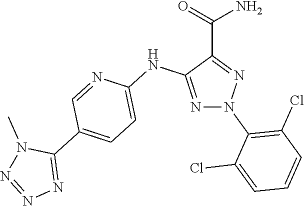 [SMILES: Cn1nnnc1-c1ccc(Nc2nn(-c3c(Cl)cccc3Cl)nc2C(N)=O)nc1] | 2-(2,6-dichlorophenyl)-5-((5-(1- methyl-1H-tetrazol-5-yl)pyridin-2- yl)amino)-2H-1,2,3-triazole-4- carboxamide |
| 159 | 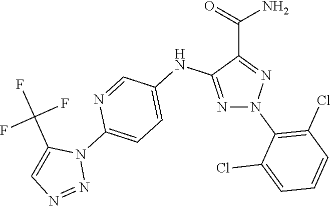 [SMILES: NC(=O)c1nn(-c2c(Cl)cccc2Cl)nc1Nc1ccc(-n2nncc2C(F)(F)F)nc1] | 2-(2,6-dichlorophenyl)-5-((6-(5- (trifluoromethyl)-1H-1,2,3-triazol-1- yl)pyridin-3-yl)amino)-2H-1,2,3- triazole-4-carboxamide |
| 160 | 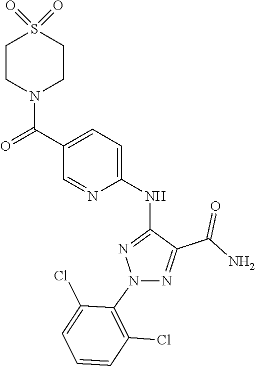 [SMILES: NC(=O)c1nn(-c2c(Cl)cccc2Cl)nc1Nc1ccc(C(=O)N2CCS(=O)(=O)CC2)cn1] | 2-(2,6-dichlorophenyl)-5-((5-(1,1- dioxidothiomorpholine-4- carbonyl)pyridin-2-yl)amino)-2H- 1,2,3-triazole-4-carboxamide |
| 161 | 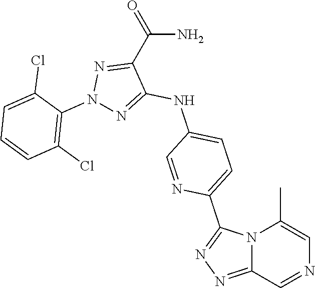 [SMILES: Cc1cncc2nnc(-c3ccc(Nc4nn(-c5c(Cl)cccc5Cl)nc4C(N)=O)cn3)n12] | 2-(2,6-dichlorophenyl)-5-((6-(5- methyl-[1,2,4]triazolo[4,3-a]pyrazin- 3-yl)pyridin-3-yl)amino)-2H-1,2,3- triazole-4-carboxamide |
| 162 | 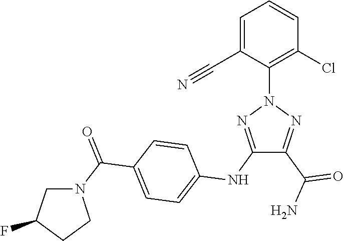 [SMILES: N#Cc1cccc(Cl)c1-n1nc(Nc2ccc(C(=O)N3CC[C@@H](F)C3)cc2)c(C(N)=O)n1] | (R)-2-(2-chloro-6-cyanophenyl)-5-((4- (3-fluoropyrrolidine-1- carbonyl)phenyl)amino)-2H-1,2,3- triazole-4-carboxamide |
| 163 | 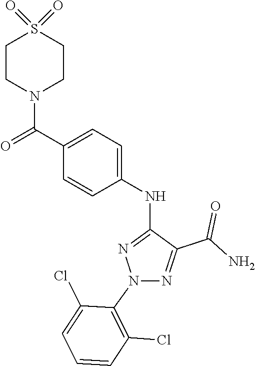 [SMILES: NC(=O)c1nn(-c2c(Cl)cccc2Cl)nc1Nc1ccc(C(=O)N2CCS(=O)(=O)CC2)cc1] | 2-(2,6-dichlorophenyl)-5-((4-(1,1- dioxidothiomorpholine-4- carbonyl)phenyl)amino)-2H-1,2,3- triazole-4-carboxamide |
| 164 | 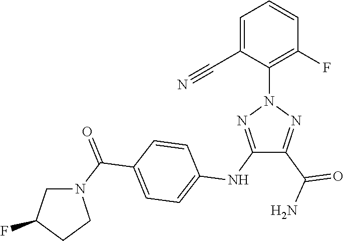 [SMILES: N#Cc1cccc(F)c1-n1nc(Nc2ccc(C(=O)N3CC[C@@H](F)C3)cc2)c(C(N)=O)n1] | (R)-2-(2-cyano-6-fluorophenyl)-5-((4- (3-fluoropyrrolidine-1- carbonyl)phenyl)amino)-2H-1,2,3- triazole-4-carboxamide |
| 165 | 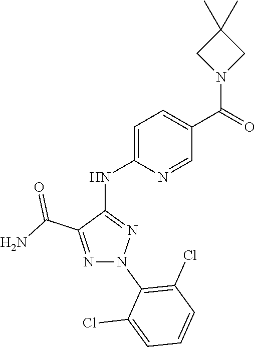 [SMILES: CC1(C)CN(C(=O)c2ccc(Nc3nn(-c4c(Cl)cccc4Cl)nc3C(N)=O)nc2)C1] | 2-(2,6-dichlorophenyl)-5-((5-(3,3- dimethylazetidine-1-carbonyl)pyridin- 2-yl)amino)-2H-1,2,3-triazole-4- carboxamide |
| 166 | 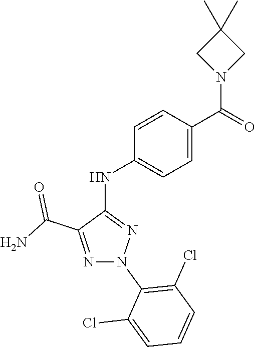 [SMILES: CC1(C)CN(C(=O)c2ccc(Nc3nn(-c4c(Cl)cccc4Cl)nc3C(N)=O)cc2)C1] | 2-(2,6-dichlorophenyl)-5-((4-(3,3- dimethylazetidine-1- carbonyl)phenyl)amino)-2H-1,2,3- triazole-4-carboxamide |
| 167 | 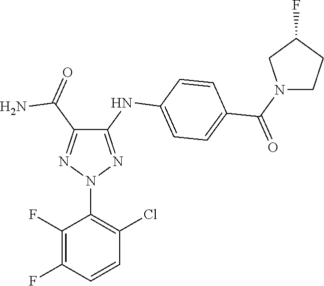 [SMILES: NC(=O)c1nn(-c2c(Cl)ccc(F)c2F)nc1Nc1ccc(C(=O)N2CC[C@@H](F)C2)cc1] | (R)-2-(6-chloro-2,3-difluorophenyl)- 5-((4-(3-fluoropyrrolidine-1- carbonyl)phenyl)amino)-2H-1,2,3- triazole-4-carboxamide |
| 168 | 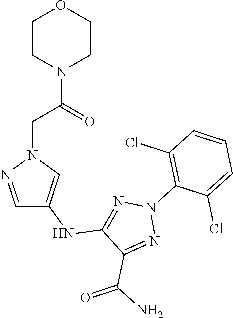 [SMILES: NC(=O)c1nn(-c2c(Cl)cccc2Cl)nc1Nc1cnn(CC(=O)N2CCOCC2)c1] | 2-(2,6-dichlorophenyl)-5-((1-(2- morpholino-2-oxoethyl)-1H-pyrazol- 4-yl)amino)-2H-1,2,3-triazole-4- carboxamide |
| 169 | 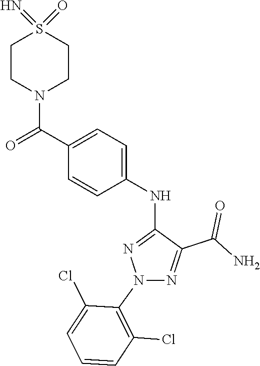 [SMILES: N=S1(=O)CCN(C(=O)c2ccc(Nc3nn(-c4c(Cl)cccc4Cl)nc3C(N)=O)cc2)CC1] | 2-(2,6-dichlorophenyl)-5-((4-(1- imino-1-oxidothiomorpholine-4- carbonyl)phenyl)amino)-2H-1,2,3- triazole-4-carboxamide |
| 170 | 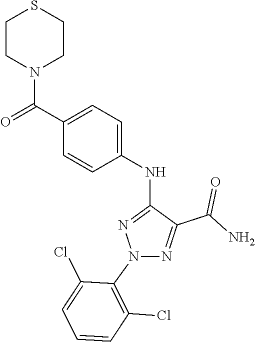 [SMILES: NC(=O)c1nn(-c2c(Cl)cccc2Cl)nc1Nc1ccc(C(=O)N2CCSCC2)cc1] | 2-(2,6-dichlorophenyl)-5-((4- (thiomorpholine-4- carbonyl)phenyl)amino)-2H-1,2,3- triazole-4-carboxamide |
| 171 | 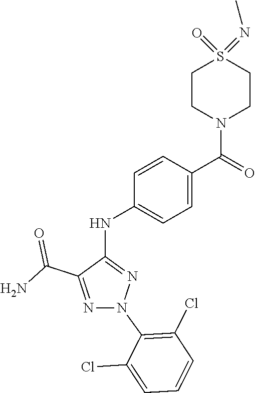 [SMILES: CN=S1(=O)CCN(C(=O)c2ccc(Nc3nn(-c4c(Cl)cccc4Cl)nc3C(N)=O)cc2)CC1] | 2-(2,6-dichlorophenyl)-5-((4-(1- (methylimino)-1- oxidothiomorpholine-4- carbonyl)phenyl)amino)-2H-1,2,3- triazole-4-carboxamide |
| 172 | 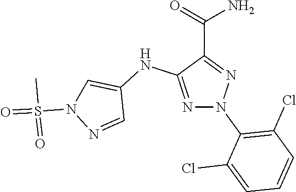 [SMILES: CS(=O)(=O)n1cc(Nc2nn(-c3c(Cl)cccc3Cl)nc2C(N)=O)cn1] | 2-(2,6-dichlorophenyl)-5-((1- (methylsulfonyl)-1H-pyrazol-4- yl)amino)-2H-1,2,3-triazole-4- carboxamide |
| 173 | 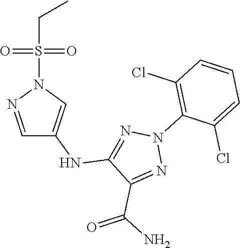 [SMILES: CCS(=O)(=O)n1cc(Nc2nn(-c3c(Cl)cccc3Cl)nc2C(N)=O)cn1] | 2-(2,6-dichlorophenyl)-5-((1- (ethylsulfonyl)-1H-pyrazol-4- yl)amino)-2H-1,2,3-triazole-4- carboxamide |
| 174 | 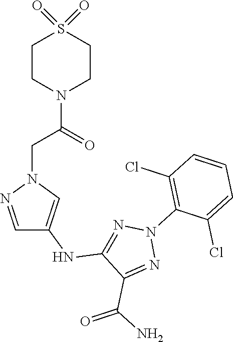 [SMILES: NC(=O)c1nn(-c2c(Cl)cccc2Cl)nc1Nc1cnn(CC(=O)N2CCS(=O)(=O)CC2)c1] | 2-(2,6-dichlorophenyl)-5-((1-(2-(1,1- dioxidothiomorpholino)-2-oxoethyl)- 1H-pyrazol-4-yl)amino)-2H-1,2,3- triazole-4-carboxamide |
| 175 | 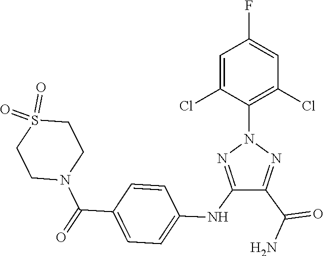 [SMILES: NC(=O)c1nn(-c2c(Cl)cc(F)cc2Cl)nc1Nc1ccc(C(=O)N2CCS(=O)(=O)CC2)cc1] | 2-(2,6-dichloro-4-fluorophenyl)-5-((4- (1,1-dioxidothiomorpholine-4- carbonyl)phenyl)amino)-2H-1,2,3- triazole-4-carboxamide |
| 176 | 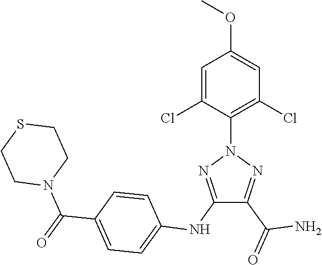 [SMILES: COc1cc(Cl)c(-n2nc(Nc3ccc(C(=O)N4CCSCC4)cc3)c(C(N)=O)n2)c(Cl)c1] | 2-(2,6-dichloro-4-methoxyphenyl)-5- ((4-(thiomorpholine-4- carbonyl)phenyl)amino)-2H-1,2,3- triazole-4-carboxamide |
| 177 | 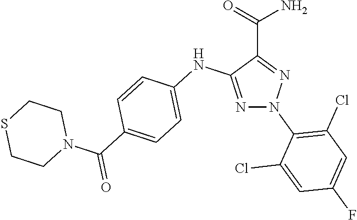 [SMILES: NC(=O)c1nn(-c2c(Cl)cc(F)cc2Cl)nc1Nc1ccc(C(=O)N2CCSCC2)cc1] | 2-(2,6-dichloro-4-fluorophenyl)-5-((4- (thiomorpholine-4- carbonyl)phenyl)amino)-2H-1,2,3- triazole-4-carboxamide |

In an aspect, the invention provides compounds of Formula (I):

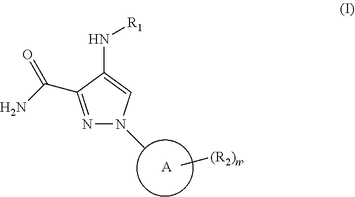

wherein:
R₁ is C(O)CH₂R₃, optionally substituted cycloalkyl, optionally substituted cycloheteroalkyl, optionally substituted cycloalkenyl, optionally substituted cycloheteroalkenyl, optionally substituted aryl, or optionally substituted heteroaryl;
each instance of R₂ is independently halo, haloalkyl, cyano, optionally substituted alkoxyl, optionally substituted alkyl, optionally substituted heteroalkyl, optionally substituted alkenyl, optionally substituted heteroalkenyl;
R₃ is optionally substituted cycloalkyl, optionally substituted cycloheteroalkyl, optionally substituted cycloalkenyl, optionally substituted cycloheteroalkenyl, optionally substituted aryl, or optionally substituted heteroaryl;
A is cycloalkyl, cycloheteroalkyl, cycloalkenyl, cycloheteroalkenyl, aryl, or heteroaryl; and
n is 0-5.

The optionally substituted aryl in R₁ may be other than a phenyl.

A may be an aryl substituted with 2 or 3 instances of R₂. A may be a phenyl substituted with 2 or 3 instances of R₂. A may be a heteroaryl substituted with 2 or 3 instances of R₂. A may be a six-membered heteroaryl substituted with 2 or 3 instances of R₂.

The compound of Formula (I) may be represented by Formula (Ia):

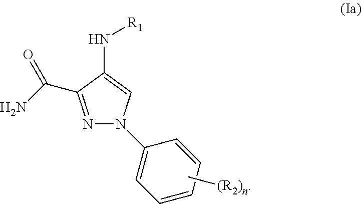

The compound of Formula (Ia) may be represented by one of Formulas (Ia-1), (Ia-2), (Ia-3), (Ia-4), (Ia-5), (Ia-6), and (Ia-7):

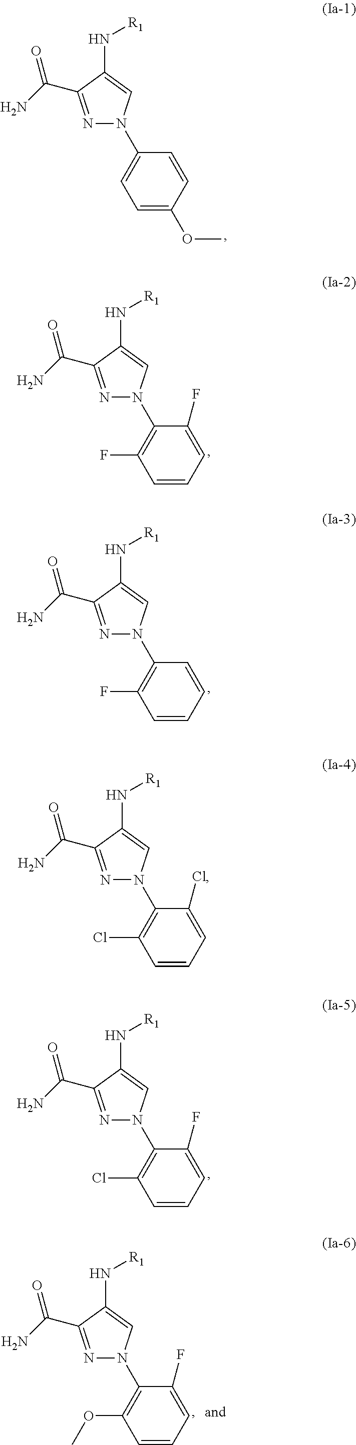

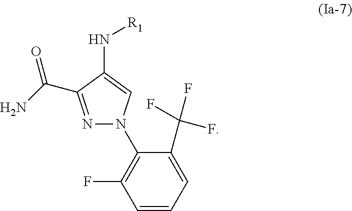

The compound of Formula (I) may be represented by Formula (Ib):

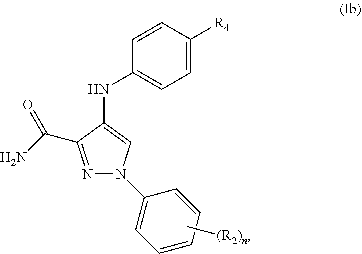

wherein:
R₄ is H, C(O)NR₅R₆, C(O)R₅, S(O)₂R₅, S(O)₂NR₅R₆, P(O)R₇R₅, OR₅, optionally substituted cycloalkyl, optionally substituted cycloheteroalkyl, optionally substituted cycloalkenyl, optionally substituted cycloheteroalkenyl, optionally substituted aryl, or optionally substituted heteroaryl;
either:
   R₅ is H, optionally substituted alkyl, optionally substituted heteroalkyl, optionally substituted cycloalkyl, optionally substituted cycloheteroalkyl, optionally substituted cycloalkenyl, optionally substituted cycloheteroalkenyl, optionally substituted aryl, or optionally substituted heteroaryl, and R₆, if present, is H, optionally substituted alkyl, or optionally substituted heteroalkyl; or
   R₅ and R₆ together form an optionally substituted cycloalkyl or optionally substituted cycloheteroalkyl;
R₇ is optionally substituted alkyl or optionally substituted heteroalkyl; and
R₈ is optionally substituted alkyl or optionally substituted heteroalkyl.
R₁ may be a heteroaryl having one of the following structures:

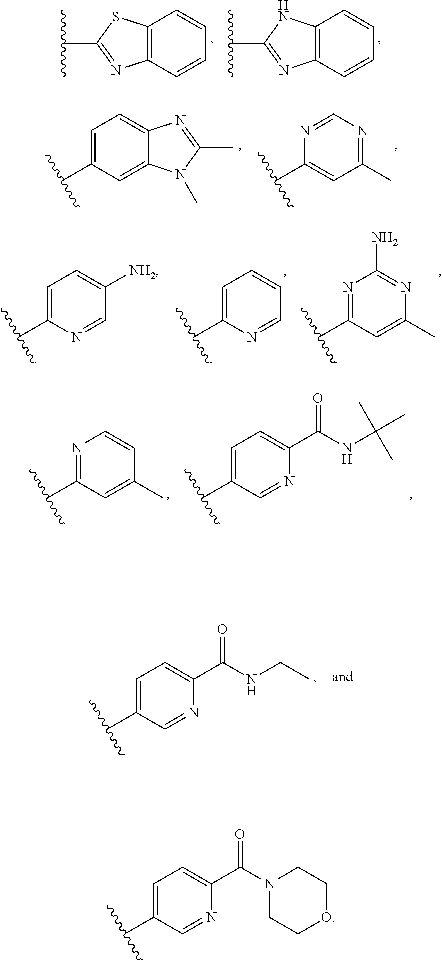

R₃ may have the following structure:

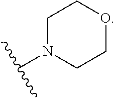 [SMILES: *N1CCOCC1]

The compound of Formula (I) may have one of the structures shown in Table 1.

TABLE 1

| Compound | | |
|---|---|---|
| No. | Structure | IUPAC-Name |
| 1 | 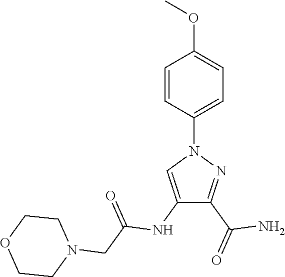 [SMILES: COc1ccc(-n2cc(NC(=O)CN3CCOCC3)c(C(N)=O)n2)cc1] | 1-(4-methoxyphenyl)-4-(2- morpholinoacetamido)-1H-pyrazole- 3-carboxamide |
| 2 | 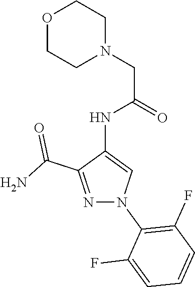 [SMILES: NC(=O)c1nn(-c2c(F)cccc2F)cc1NC(=O)CN1CCOCC1] | 1-(2,6-difluorophenyl)-4-(2- morpholinoacetamido)-1H-pyrazole- 3-carboxamide |
| 3 | 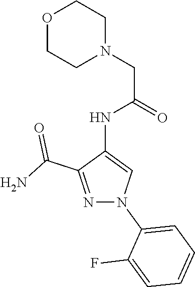 [SMILES: NC(=O)c1nn(-c2ccccc2F)cc1NC(=O)CN1CCOCC1] | 1-(2-fluorophenyl)-4-(2- morpholinoacetamido)-1H-pyrazole- 3-carboxamide |
| 4 | 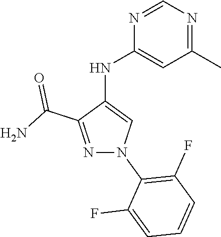 [SMILES: Cc1cc(Nc2cn(-c3c(F)cccc3F)nc2C(N)=O)ncn1] | 1-(2,6-difluorophenyl)-4-((6- methylpyrimidin-4-yl)amino)-1H- pyrazole-3-carboxamide |
| 5 | 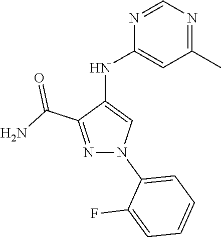 [SMILES: Cc1cc(Nc2cn(-c3ccccc3F)nc2C(N)=O)ncn1] | 1-(2-fluorophenyl)-4-((6- methylpyrimidin-4-yl)amino)-1H- pyrazole-3-carboxamide |
| 6 | 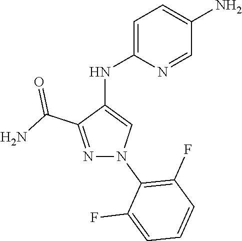 [SMILES: NC(=O)c1nn(-c2c(F)cccc2F)cc1Nc1ccc(N)cn1] | 4-((5-aminopyridin-2-yl)amino)-1- (2,6-difluorophenyl)-1H-pyrazole-3- carboxamide |
| 7 | 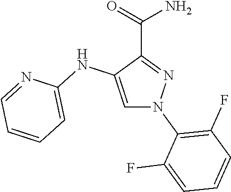 [SMILES: NC(=O)c1nn(-c2c(F)cccc2F)cc1Nc1ccccn1] | 1-(2,6-difluorophenyl)-4-(pyridin-2- ylamino)-1H-pyrazole-3-carboxamide |
| 8 | 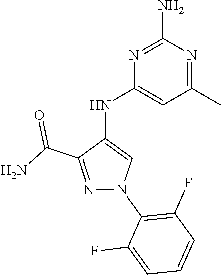 [SMILES: Cc1cc(Nc2cn(-c3c(F)cccc3F)nc2C(N)=O)nc(N)n1] | 4-((2-amino-6-methylpyrimidin-4- yl)amino)-1-(2,6-difluorophenyl)-1H- pyrazole-3-carboxamide |
| 9 | 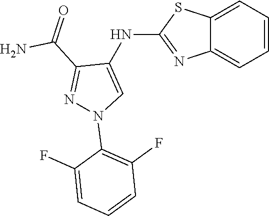 [SMILES: NC(=O)c1nn(-c2c(F)cccc2F)cc1Nc1nc2ccccc2s1] | 4-(benzo[d]thiazol-2-ylamino)-1-(2,6- difluorophenyl)-1H-pyrazole-3- carboxamide |
| 10 | 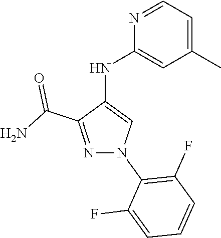 [SMILES: Cc1ccnc(Nc2cn(-c3c(F)cccc3F)nc2C(N)=O)c1] | 1-(2,6-difluorophenyl)-4-((4- methylpyridin-2-yl)amino)-1H- pyrazole-3-carboxamide |
| 11 | 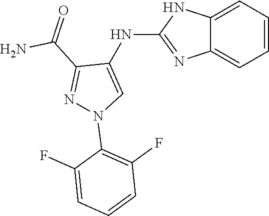 [SMILES: NC(=O)c1nn(-c2c(F)cccc2F)cc1Nc1nc2ccccc2[nH]1] | 4-((1H-benzo[d]imidazol-2- yl)amino)-1-(2,6-difluorophenyl)-1H- pyrazole-3-carboxamide |
| 12 | 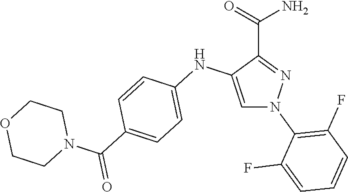 [SMILES: NC(=O)c1nn(-c2c(F)cccc2F)cc1Nc1ccc(C(=O)N2CCOCC2)cc1] | 1-(2,6-difluorophenyl)-4-((4- (morpholine-4- carbonyl)phenyl)amino)-1H-pyrazole- 3-carboxamide |
| 13 | 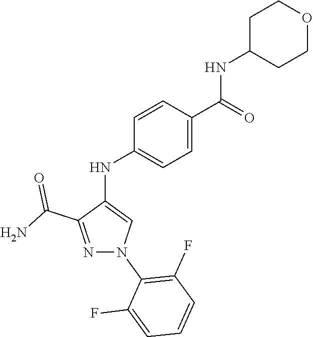 [SMILES: NC(=O)c1nn(-c2c(F)cccc2F)cc1Nc1ccc(C(=O)NC2CCOCC2)cc1] | 1-(2,6-difluorophenyl)-4-((4- ((tetrahydro-2H-pyran-4- yl)carbamoyl)phenyl)amino)-1H- pyrazole-3-carboxamide |
| 14 | 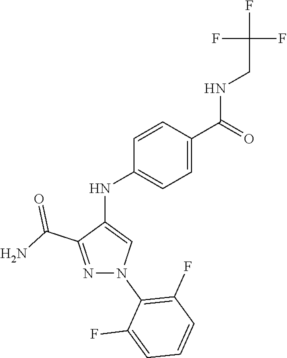 [SMILES: NC(=O)c1nn(-c2c(F)cccc2F)cc1Nc1ccc(C(=O)NCC(F)(F)F)cc1] | 1-(2,6-difluorophenyl)-4-((4-((2,2,2- trifluoroethyl)carbamoyl)phenyl) amino)-1H-pyrazole-3-carboxamide |
| 15 | 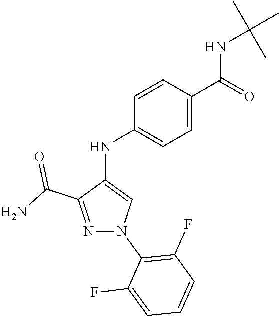 [SMILES: CC(C)(C)NC(=O)c1ccc(Nc2cn(-c3c(F)cccc3F)nc2C(N)=O)cc1] | 4-((4-(tert- butylcarbamoyl)phenyl)amino)-1- (2,6-difluorophenyl)-1H-pyrazole-3- carboxamide |
| 16 | 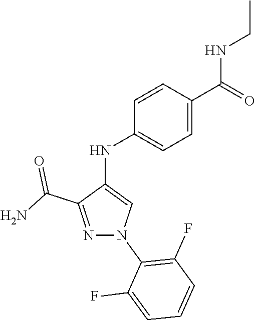 [SMILES: CCNC(=O)c1ccc(Nc2cn(-c3c(F)cccc3F)nc2C(N)=O)cc1] | 1-(2,6-difluorophenyl)-4-((4- (ethylcarbamoyl)phenyl)amino)-1H- pyrazole-3-carboxamide |
| 17 | 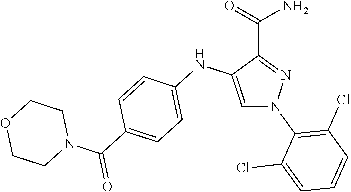 [SMILES: NC(=O)c1nn(-c2c(Cl)cccc2Cl)cc1Nc1ccc(C(=O)N2CCOCC2)cc1] | 1-(2,6-dichlorophenyl)-4-((4- (morpholine-4- carbonyl)phenyl)amino)-1H-pyrazole- 3-carboxamide |
| 18 | 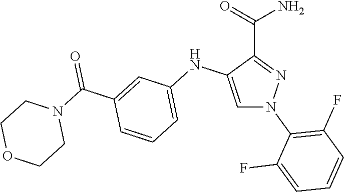 [SMILES: NC(=O)c1nn(-c2c(F)cccc2F)cc1Nc1cccc(C(=O)N2CCOCC2)c1] | 1-(2,6-difluorophenyl)-4-((3- (morpholine-4- carbonyl)phenyl)amino)-1H-pyrazole- 3-carboxamide |
| 19 | 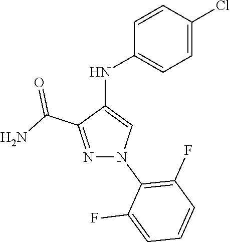 [SMILES: NC(=O)c1nn(-c2c(F)cccc2F)cc1Nc1ccc(Cl)cc1] | 4-((4-chlorophenyl)amino)-1-(2,6- difluorophenyl)-1H-pyrazole-3- carboxamide |
| 20 | 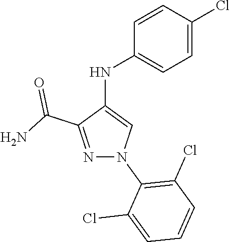 [SMILES: NC(=O)c1nn(-c2c(Cl)cccc2Cl)cc1Nc1ccc(Cl)cc1] | 4-((4-chlorophenyl)amino)-1-(2,6- dichlorophenyl)-1H-pyrazole-3- carboxamide |
| 21 | 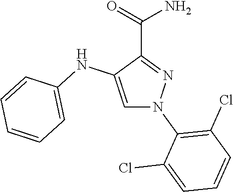 [SMILES: NC(=O)c1nn(-c2c(Cl)cccc2Cl)cc1Nc1ccccc1] | 1-(2,6-dichlorophenyl)-4- (phenylamino)-1H-pyrazole-3- carboxamide |
| 22 | 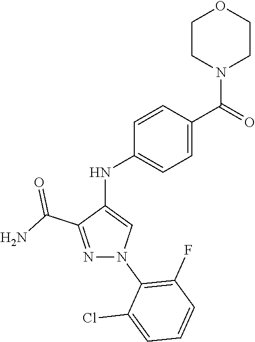 [SMILES: NC(=O)c1nn(-c2c(F)cccc2Cl)cc1Nc1ccc(C(=O)N2CCOCC2)cc1] | 1-(2-chloro-6-fluorophenyl)-4-((4- (morpholine-4- carbonyl)phenyl)amino)-1H-pyrazole- 3-carboxamide |
| 23 | 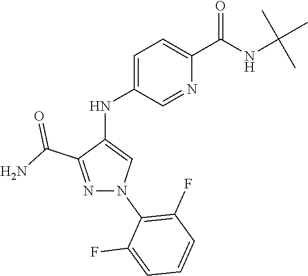 [SMILES: CC(C)(C)NC(=O)c1ccc(Nc2cn(-c3c(F)cccc3F)nc2C(N)=O)cn1] | N-(tert-butyl)-5-((3-carbamoyl-1-(2,6- difluorophenyl)-1H-pyrazol-4- yl)amino)picolinamide |
| 24 | 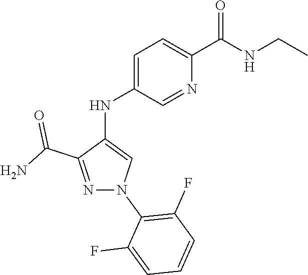 [SMILES: CCNC(=O)c1ccc(Nc2cn(-c3c(F)cccc3F)nc2C(N)=O)cn1] | 5-((3-carbamoyl-1-(2,6- difluorophenyl)-1H-pyrazol-4- yl)amino)-N-ethylpicolinamide |
| 25 | 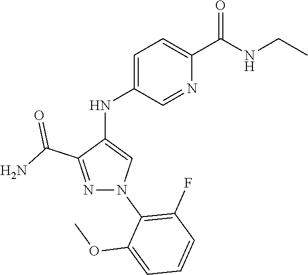 [SMILES: CCNC(=O)c1ccc(Nc2cn(-c3c(F)cccc3OC)nc2C(N)=O)cn1] | 5-((3-carbamoyl-1-(2-fluoro-6- methoxyphenyl)-1H-pyrazol-4- yl)amino)-N-ethylpicolinamide |
| 26 | 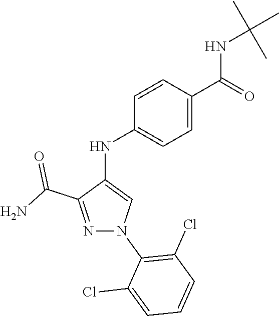 [SMILES: CC(C)(C)NC(=O)c1ccc(Nc2cn(-c3c(Cl)cccc3Cl)nc2C(N)=O)cc1] | 4-((4-(tert- butylcarbamoyl)phenyl)amino)-1- (2,6-dichlorophenyl)-1H-pyrazole-3- carboxamide |
| 27 | 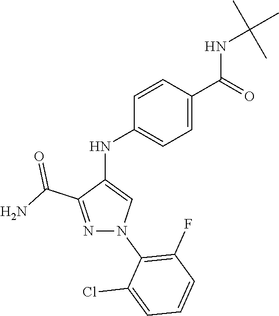 [SMILES: CC(C)(C)NC(=O)c1ccc(Nc2cn(-c3c(F)cccc3Cl)nc2C(N)=O)cc1] | 4-((4-(tert- butylcarbamoyl)phenyl)amino)-1-(2- chloro-6-fluorophenyl)-1H-pyrazole- 3-carboxamide |
| 28 | 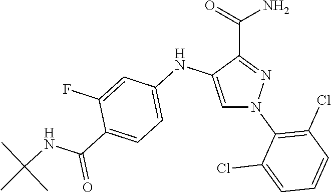 [SMILES: CC(C)(C)NC(=O)c1ccc(Nc2cn(-c3c(Cl)cccc3Cl)nc2C(N)=O)cc1F] | 4-((4-(tert-butylcarbamoyl)-3- fluorophenyl)amino)-1-(2,6- dichlorophenyl)-1H-pyrazole-3- carboxamide |
| 29 | 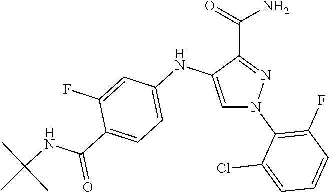 [SMILES: CC(C)(C)NC(=O)c1ccc(Nc2cn(-c3c(F)cccc3Cl)nc2C(N)=O)cc1F] | 4-((4-tert-butylcarbamoyl)-3- fluorophenyl)amino)-1-(2-chloro-6- fluorophenyl)-1H-pyrazole-3- carboxamide |
| 30 | 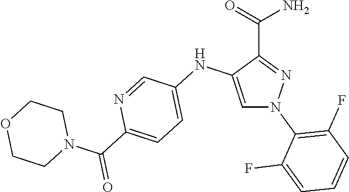 [SMILES: NC(=O)c1nn(-c2c(F)cccc2F)cc1Nc1ccc(C(=O)N2CCOCC2)nc1] | 1-(2,6-difluorophenyl)-4-((6- (morpholine-4-carbonyl)pyridin-3- yl)amino)-1H-pyrazole-3- carboxamide |
| 31 | 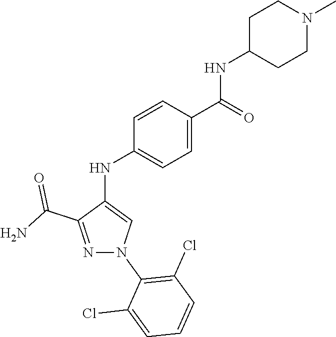 [SMILES: CN1CCC(NC(=O)c2ccc(Nc3cn(-c4c(Cl)cccc4Cl)nc3C(N)=O)cc2)CC1] | 1-(2,6-dichlorophenyl)-4-((4-((1- methylpiperidin-4- yl)carbamoyl)phenyl)amino)-1H- pyrazole-3-carboxamide |
| 32 | 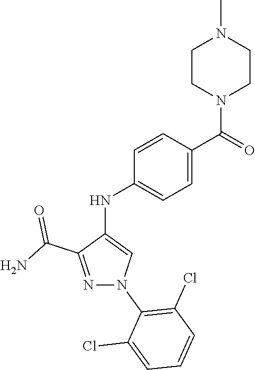 [SMILES: CN1CCN(C(=O)c2ccc(Nc3cn(-c4c(Cl)cccc4Cl)nc3C(N)=O)cc2)CC1] | 1-(2,6-dichlorophenyl)-4-((4-(4- methylpiperazine-1- carbonyl)phenyl)amino)-1H-pyrazole- 3-carboxamide |
| 33 | 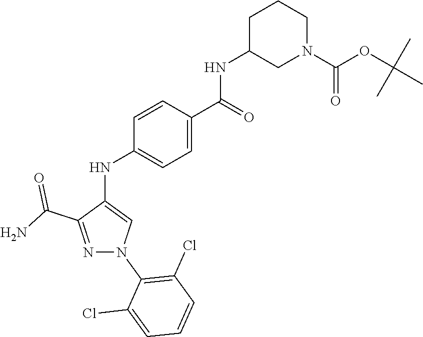 [SMILES: CC(C)(C)OC(=O)N1CCCC(NC(=O)c2ccc(Nc3cn(-c4c(Cl)cccc4Cl)nc3C(N)=O)cc2)C1] | tert-butyl_3-(4-((3-carbamoyl-1-(2,6- dichlorophenyl)-1H-pyrazol-4- yl)amino)benzamido)piperidine-1- carboxylate |
| 34 | 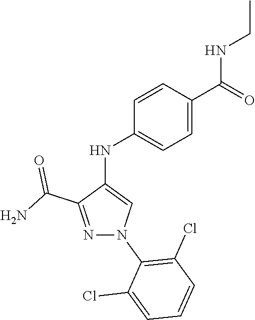 [SMILES: CCNC(=O)c1ccc(Nc2cn(-c3c(Cl)cccc3Cl)nc2C(N)=O)cc1] | 1-(2,6-dichlorophenyl)-4-((4- (ethylcarbamoyl)phenyl)amino)-1H- pyrazole-3-carboxamide |
| 35 | 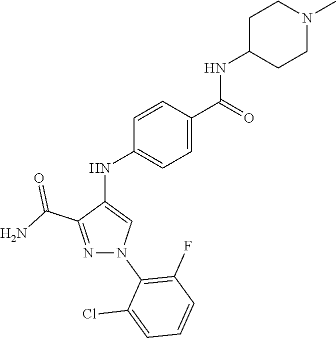 [SMILES: CN1CCC(NC(=O)c2ccc(Nc3cn(-c4c(F)cccc4Cl)nc3C(N)=O)cc2)CC1] | 1-(2-chloro-6-fluorophenyl)-4-((4-((1- methylpiperidin-4- yl)carbamoyl)phenyl)amino)-1H- pyrazole-3-carboxamide |
| 36 | 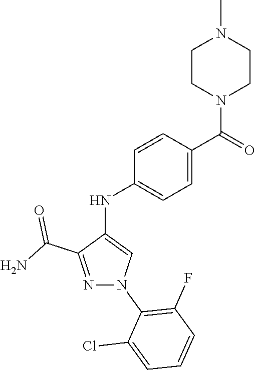 [SMILES: CN1CCN(C(=O)c2ccc(Nc3cn(-c4c(F)cccc4Cl)nc3C(N)=O)cc2)CC1] | 1-(2-chloro-6-fluorophenyl)-4-((4-(4- methylpiperazine-1- carbonyl)phenyl)amino)-1H-pyrazole- 3-carboxamide |
| 37 | 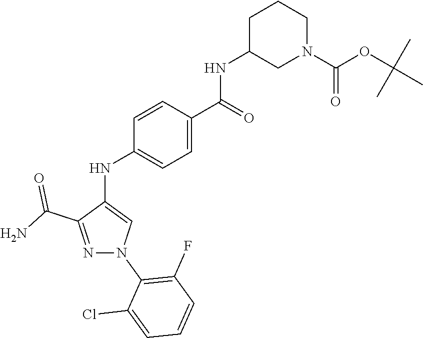 [SMILES: CC(C)(C)OC(=O)N1CCCC(NC(=O)c2ccc(Nc3cn(-c4c(F)cccc4Cl)nc3C(N)=O)cc2)C1] | tert-butyl_3-(4-((3-carbamoyl-1-(2- chloro-6-fluorophenyl)-1H-pyrazol-4- yl)amino)benzamido)piperidine-1- carboxylate |
| 38 | 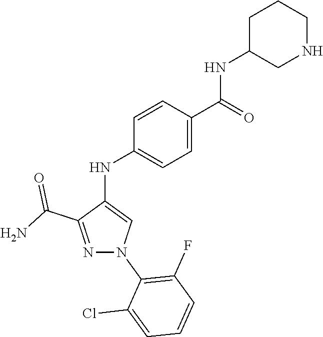 [SMILES: NC(=O)c1nn(-c2c(F)cccc2Cl)cc1Nc1ccc(C(=O)NC2CCCNC2)cc1] | 1-(2-chloro-6-fluorophenyl)-4-((4- (piperidin-3- ylcarbamoyl)phenyl)amino)-1H- pyrazole-3-carboxamide |
| 39 | 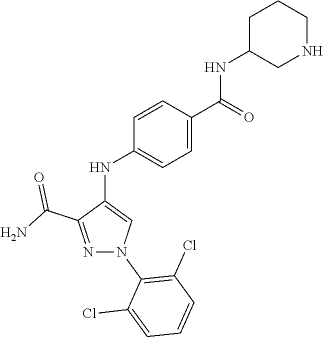 [SMILES: NC(=O)c1nn(-c2c(Cl)cccc2Cl)cc1Nc1ccc(C(=O)NC2CCCNC2)cc1] | 1-(2,6-dichlorophenyl)-4-((4- (piperidin-3- ylcarbamoyl)phenyl)amino)-1H- pyrazole-3-carboxamide |
| 40 | 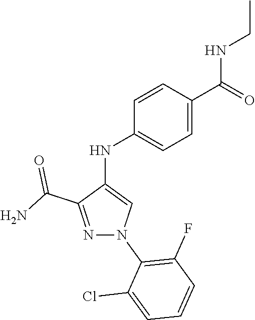 [SMILES: CCNC(=O)c1ccc(Nc2cn(-c3c(F)cccc3Cl)nc2C(N)=O)cc1] | 1-(2-chloro-6-fluorophenyl)-4-((4- (ethylcarbamoyl)phenyl)amino)-1H- pyrazole-3-carboxamide |
| 41 | 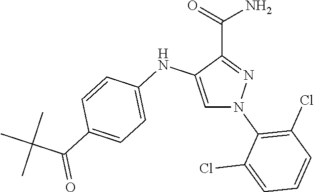 [SMILES: CC(C)(C)C(=O)c1ccc(Nc2cn(-c3c(Cl)cccc3Cl)nc2C(N)=O)cc1] | 1-(2,6-dichlorophenyl)-4-((4- pivaloylphenyl)amino)-1H-pyrazole-3- carboxamide |
| 42 | 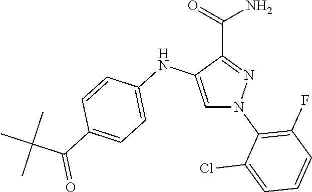 [SMILES: CC(C)(C)C(=O)c1ccc(Nc2cn(-c3c(F)cccc3Cl)nc2C(N)=O)cc1] | 1-(2-chloro-6-fluorophenyl)-4-((4- pivaloylphenyl)amino)-1H-pyrazole- 3-carboxamide |
| 43 | 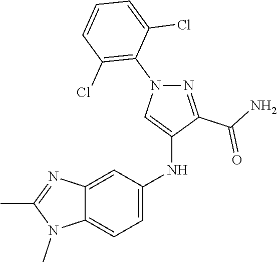 [SMILES: Cc1nc2cc(Nc3cn(-c4c(Cl)cccc4Cl)nc3C(N)=O)ccc2n1C] | 1-(2,6-dichlorophenyl)-4-((1,2- dimethyl-1H-benzo[d]imidazol-5- yl)amino)-1H-pyrazole-3- carboxamide |
| 44 | 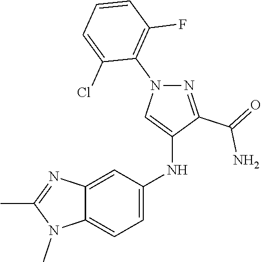 [SMILES: Cc1nc2cc(Nc3cn(-c4c(F)cccc4Cl)nc3C(N)=O)ccc2n1C] | 1-(2-chloro-6-fluorophenyl)-4-((1,2- dimethyl-1H-benzo[d]imidazol-5- yl)amino)-1H-pyrazole-3- carboxamide |
| 45 | 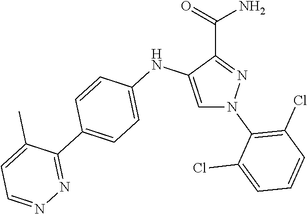 [SMILES: Cc1ccnnc1-c1ccc(Nc2cn(-c3c(Cl)cccc3Cl)nc2C(N)=O)cc1] | 1-(2,6-dichlorophenyl)-4-((4-(4- methylpyridazin-3-yl)phenyl)amino)- 1H-pyrazole-3-carboxamide |
| 46 | 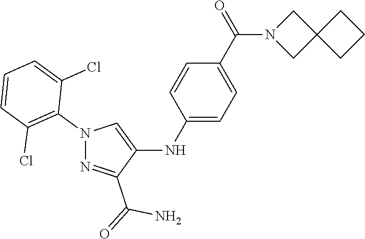 [SMILES: NC(=O)c1nn(-c2c(Cl)cccc2Cl)cc1Nc1ccc(C(=O)N2CC3(CCC3)C2)cc1] | 4-((4-(2-azaspiro[3.3]heptane-2- carbonyl)phenyl)amino)-1-(2,6- dichlorophenyl)-1H-pyrazole-3- carboxamide |
| 47 | 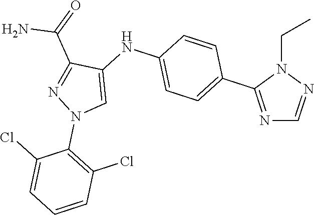 [SMILES: CCn1ncnc1-c1ccc(Nc2cn(-c3c(Cl)cccc3Cl)nc2C(N)=O)cc1] | 1-(2,6-dichlorophenyl)-4-((4-(1-ethyl- 1H-1,2,4-triazol-5-yl)phenyl)amino)- 1H-pyrazole-3-carboxamide |
| 48 | 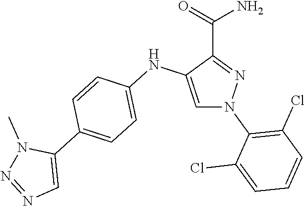 [SMILES: Cn1nncc1-c1ccc(Nc2cn(-c3c(Cl)cccc3Cl)nc2C(N)=O)cc1] | 1-(2,6-dichlorophenyl)-4-((4-(1- methyl-1H-1,2,3-triazol-5- yl)phenyl)amino)-1H-pyrazole-3- carboxamide |
| 49 | 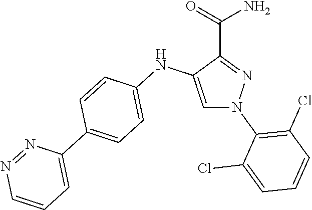 [SMILES: NC(=O)c1nn(-c2c(Cl)cccc2Cl)cc1Nc1ccc(-c2cccnn2)cc1] | 1-(2,6-dichlorophenyl)-4-((4- (pyridazin-3-yl)phenyl)amino)-1H- pyrazole-3-carboxamide |
| 50 | 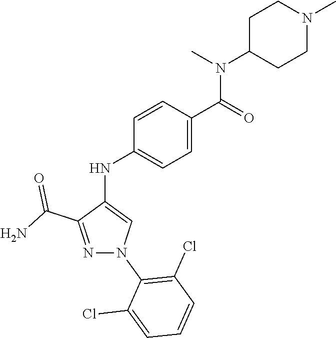 [SMILES: CN1CCC(N(C)C(=O)c2ccc(Nc3cn(-c4c(Cl)cccc4Cl)nc3C(N)=O)cc2)CC1] | 1-(2,6-dichlorophenyl)-4-((4- (methyl(1-methylpiperidin-4- yl)carbamoyl)phenyl)amino)-1H- pyrazole-3-carboxamide |
| 51 | 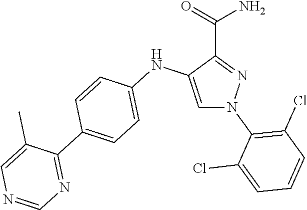 [SMILES: Cc1cncnc1-c1ccc(Nc2cn(-c3c(Cl)cccc3Cl)nc2C(N)=O)cc1] | 1-(2,6-dichlorophenyl)-4-((4-(5- methylpyrimidin-4-yl)phenyl)amino)- 1H-pyrazole-3-carboxamide |
| 52 | 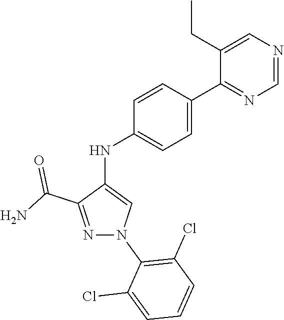 [SMILES: CCc1cncnc1-c1ccc(Nc2cn(-c3c(Cl)cccc3Cl)nc2C(N)=O)cc1] | 1-(2,6-dichlorophenyl)-4-((4-(5- ethylpyrimidin-4-yl)phenyl)amino)- 1H-pyrazole-3-carboxamide |
| 53 | 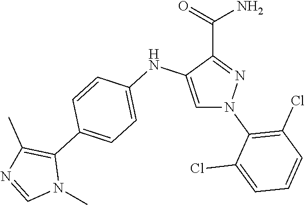 [SMILES: Cc1ncn(C)c1-c1ccc(Nc2cn(-c3c(Cl)cccc3Cl)nc2C(N)=O)cc1] | 1-(2,6-dichlorophenyl)-4-((4-(1,4- dimethyl-1H-imidazol-5- yl)phenyl)amino)-1H-pyrazole-3- carboxamide |
| 54 | 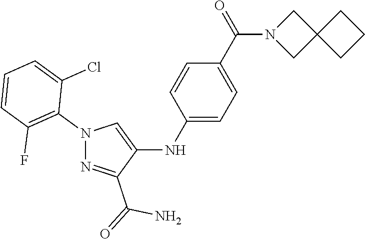 [SMILES: NC(=O)c1nn(-c2c(F)cccc2Cl)cc1Nc1ccc(C(=O)N2CC3(CCC3)C2)cc1] | 4-((4-(2-azaspiro[3.3]heptane-2- carbonyl)phenyl)amino)-1-(2-chloro- 6-fluorophenyl)-1H-pyrazole-3- carboxamide |
| 55 | 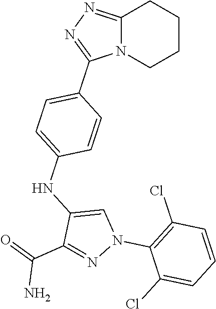 [SMILES: NC(=O)c1nn(-c2c(Cl)cccc2Cl)cc1Nc1ccc(-c2nnc3n2CCCC3)cc1] | 1-(2,6-dichlorophenyl)-4-((4-(5,6,7,8- tetrahydro-[1,2,4]triazolo[4,3- a]pyridin-3-yl)phenyl)amino)-1H- pyrazole-3-carboxamide |
| 56 | 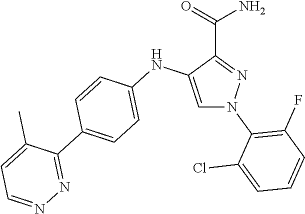 [SMILES: Cc1ccnnc1-c1ccc(Nc2cn(-c3c(F)cccc3Cl)nc2C(N)=O)cc1] | 1-(2-chloro-6-fluorophenyl)-4-((4-(4- methylpyridazin-3-yl)phenyl)amino)- 1H-pyrazole-3-carboxamide |
| 57 | 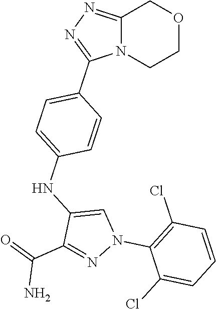 [SMILES: NC(=O)c1nn(-c2c(Cl)cccc2Cl)cc1Nc1ccc(-c2nnc3n2CCOC3)cc1] | 1-(2,6-dichlorophenyl)-4-((4-(6,8- dihydro-5H-[1,2,4]triazolo[3,4- c][1,4]oxazin-3-yl)phenyl)amino)-1H- pyrazole-3-carboxamide |
| 58 | 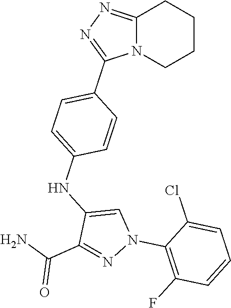 [SMILES: NC(=O)c1nn(-c2c(F)cccc2Cl)cc1Nc1ccc(-c2nnc3n2CCCC3)cc1] | 1-(2-chloro-6-fluorophenyl)-4-((4- (5,6,7,8-tetrahydro-[1,2,4]triazolo[4,3- a]pyridin-3-yl)phenyl)amino)-1H- pyrazole-3-carboxamide |
| 59 | 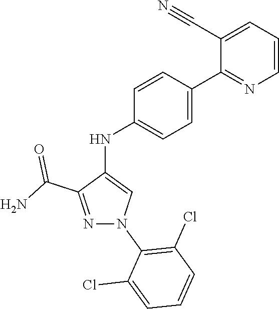 [SMILES: N#Cc1cccnc1-c1ccc(Nc2cn(-c3c(Cl)cccc3Cl)nc2C(N)=O)cc1] | 4-((4-(3-cyanopyridin-2- yl)phenyl)amino)-1-(2,6- dichlorophenyl)-1H-pyrazole-3- carboxamide |
| 60 | 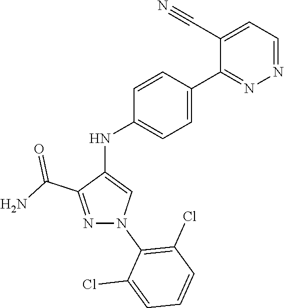 [SMILES: N#Cc1ccnnc1-c1ccc(Nc2cn(-c3c(Cl)cccc3Cl)nc2C(N)=O)cc1] | 4-((4-(4-cyanopyridazin-3- yl)phenyl)amino)-1-(2,6- dichlorophenyl)-1H-pyrazole-3- carboxamide |
| 61 | 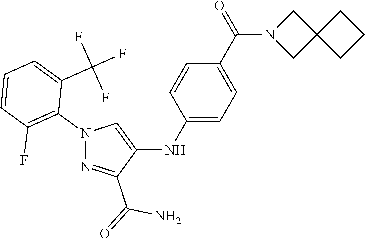 [SMILES: NC(=O)c1nn(-c2c(F)cccc2C(F)(F)F)cc1Nc1ccc(C(=O)N2CC3(CCC3)C2)cc1] | 4-((4-(2-azaspiro[3.3]heptane-2- carbonyl)phenyl)amino)-1-(2-fluoro- 6-(trifluoromethyl)phenyl)-1H- pyrazole-3-carboxamide |
| 62 | 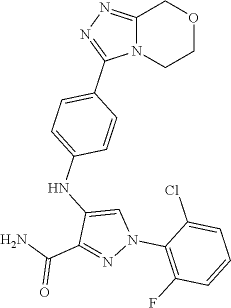 [SMILES: NC(=O)c1nn(-c2c(F)cccc2Cl)cc1Nc1ccc(-c2nnc3n2CCOC3)cc1] | 1-(2-chloro-6-fluorophenyl)-4-((4- (6,8-dihydro-5H-[1,2,4]triazolo[3,4- c][1,4]oxazin-3-yl)phenyl)amino)-1H- pyrazole-3-carboxamide |
| 63 | 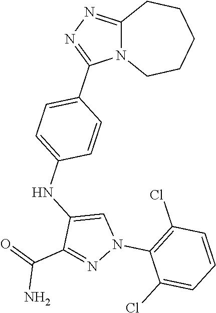 [SMILES: NC(=O)c1nn(-c2c(Cl)cccc2Cl)cc1Nc1ccc(-c2nnc3n2CCCCC3)cc1] | 1-(2,6-dichlorophenyl)-4-((4-(6,7,8,9- tetrahydro-5H-[1,2,4]triazolo[4,3- a]azepin-3-yl)phenyl)amino)-1H- pyrazole-3-carboxamide |
| 64 | 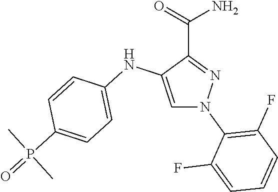 [SMILES: CP(C)(=O)c1ccc(Nc2cn(-c3c(F)cccc3F)nc2C(N)=O)cc1] | 1-(2,6-difluorophenyl)-4-((4- (dimethylphosphoryl)phenyl)amino)- 1H-pyrazole-3-carboxamide |
| 65 | 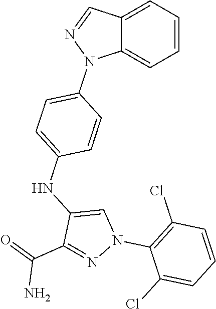 [SMILES: NC(=O)c1nn(-c2c(Cl)cccc2Cl)cc1Nc1ccc(-n2ncc3ccccc32)cc1] | 4-((4-(1H-indazol-1- yl)phenyl)amino)-1-(2,6- dichlorophenyl)-1H-pyrazole-3- carboxamide |
| 66 | 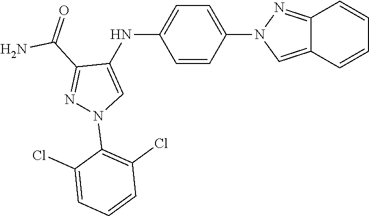 [SMILES: NC(=O)c1nn(-c2c(Cl)cccc2Cl)cc1Nc1ccc(-n2cc3ccccc3n2)cc1] | 4-((4-(2H-indazol-2- yl)phenyl)amino)-1-(2,6- dichlorophenyl)-1H-pyrazole-3- carboxamide |
| 67 | 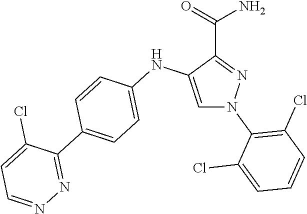 [SMILES: NC(=O)c1nn(-c2c(Cl)cccc2Cl)cc1Nc1ccc(-c2nnccc2Cl)cc1] | 4-((4-(4-chloropyridazin-3- yl)phenyl)amino)-1-(2,6- dichlorophenyl)-1H-pyrazole-3- carboxamide |
| 68 | 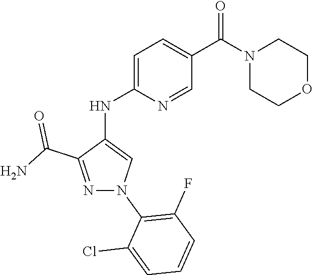 [SMILES: NC(=O)c1nn(-c2c(F)cccc2Cl)cc1Nc1ccc(C(=O)N2CCOCC2)cn1] | 1-(2-chloro-6-fluorophenyl)-4-((5- (morpholine-4-carbonyl)pyridin-2- yl)amino)-1H-pyrazole-3- carboxamide |
| 69 | 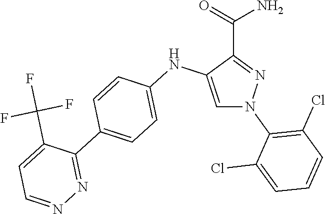 [SMILES: NC(=O)c1nn(-c2c(Cl)cccc2Cl)cc1Nc1ccc(-c2nnccc2C(F)(F)F)cc1] | 1-(2,6-dichlorophenyl)-4-((4-(4- (trifluoromethyl)pyridazin-3- yl)phenyl)amino)-1H-pyrazole-3- carboxamide |
| 70 | 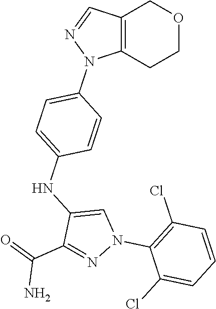 [SMILES: NC(=O)c1nn(-c2c(Cl)cccc2Cl)cc1Nc1ccc(-n2ncc3c2CCOC3)cc1] | 1-(2,6-dichlorophenyl)-4-((4-(6,7- dihydropyrano[4,3-c]pyrazol-1(4H)- yl)phenyl)amino)-1H-pyrazole-3- carboxamide |
| 71 | 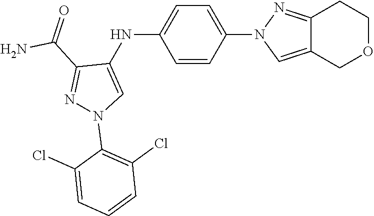 [SMILES: NC(=O)c1nn(-c2c(Cl)cccc2Cl)cc1Nc1ccc(-n2cc3c(n2)CCOC3)cc1] | 1-(2,6-dichlorophenyl)-4-((4-(6,7- dihydropyrano[4,3-c]pyrazol-2(4H)- yl)phenyl)amino)-1H-pyrazole-3- carboxamide |
| 72 | 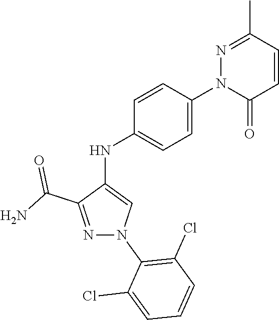 [SMILES: Cc1ccc(=O)n(-c2ccc(Nc3cn(-c4c(Cl)cccc4Cl)nc3C(N)=O)cc2)n1] | 1-(2,6-dichlorophenyl)-4-((4-(3- methyl-6-oxopyridazin-1(6H)- yl)phenyl)amino)-1H-pyrazole-3- carboxamide |
| 73 | 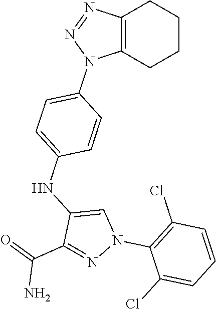 [SMILES: NC(=O)c1nn(-c2c(Cl)cccc2Cl)cc1Nc1ccc(-n2nnc3c2CCCC3)cc1] | 1-(2,6-dichlorophenyl)-4-((4-(4,5,6,7- tetrahydro-1H-benzo[d][1,2,3]triazol- 1-yl)phenyl)amino)-1H-pyrazole-3- carboxamide |
| 74 | 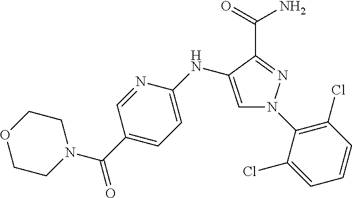 [SMILES: NC(=O)c1nn(-c2c(Cl)cccc2Cl)cc1Nc1ccc(C(=O)N2CCOCC2)cn1] | 1-(2,6-dichlorophenyl)-4-((5- (morpholine-4-carbonyl)pyridin-2- yl)amino)-1H-pyrazole-3- carboxamide |
| 75 | 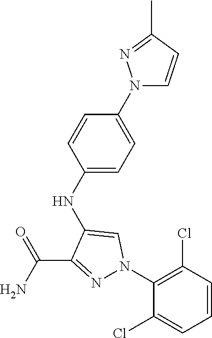 [SMILES: Cc1ccn(-c2ccc(Nc3cn(-c4c(Cl)cccc4Cl)nc3C(N)=O)cc2)n1] | 1-(2,6-dichlorophenyl)-4-((4-(3- methyl-1H-pyrazol-1- yl)phenyl)amino)-1H-pyrazole-3- carboxamide |
| 76 | 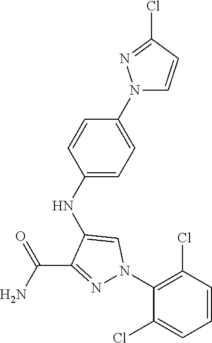 [SMILES: NC(=O)c1nn(-c2c(Cl)cccc2Cl)cc1Nc1ccc(-n2ccc(Cl)n2)cc1] | 4-((4-(3-chloro-1H-pyrazol-1- yl)phenyl)amino)-1-(2,6- dichlorophenyl)-1H-pyrazole-3- carboxamide |
| 77 | 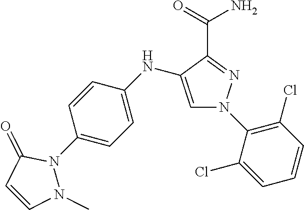 [SMILES: Cn1ccc(=O)n1-c1ccc(Nc2cn(-c3c(Cl)cccc3Cl)nc2C(N)=O)cc1] | 1-(2,6-dichlorophenyl)-4-((4-(2- methyl-5-oxo-2,5-dihydro-1H- pyrazol-1-yl)phenyl)amino)-1H- pyrazole-3-carboxamide |
| 78 | 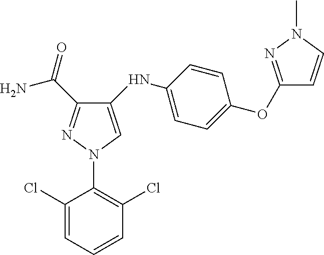 [SMILES: Cn1ccc(Oc2ccc(Nc3cn(-c4c(Cl)cccc4Cl)nc3C(N)=O)cc2)n1] | 1-(2,6-dichlorophenyl)-4-((4-((1- methyl-1H-pyrazol-3- yl)oxy)phenyl)amino)-1H-pyrazole-3- carboxamide |
| 79 | 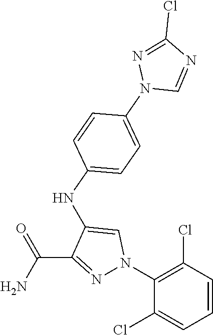 [SMILES: NC(=O)c1nn(-c2c(Cl)cccc2Cl)cc1Nc1ccc(-n2cnc(Cl)n2)cc1] | 4-((4-(3-chloro-1H-1,2,4-triazol-1- yl)phenyl)amino)-1-(2,6- dichlorophenyl)-1H-pyrazole-3- carboxamide |
| 80 | 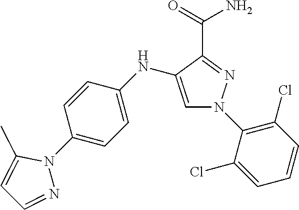 [SMILES: Cc1ccnn1-c1ccc(Nc2cn(-c3c(Cl)cccc3Cl)nc2C(N)=O)cc1] | 1-(2,6-dichlorophenyl)-4-((4-(5- methyl-1H-pyrazol-1- yl)phenyl)amino)-1H-pyrazole-3- carboxamide |
| 81 | 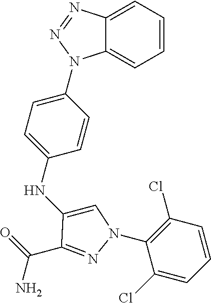 [SMILES: NC(=O)c1nn(-c2c(Cl)cccc2Cl)cc1Nc1ccc(-n2nnc3ccccc32)cc1] | 4-((4-(1H-benzo[d][1,2,3]triazol-1- yl)phenyl)amino)-1-(2,6- dichlorophenyl)-1H-pyrazole-3- carboxamide |
| 82 | 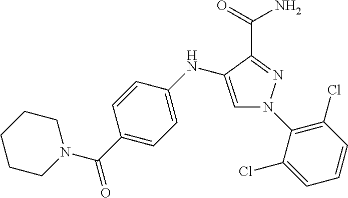 [SMILES: NC(=O)c1nn(-c2c(Cl)cccc2Cl)cc1Nc1ccc(C(=O)N2CCCCC2)cc1] | 1-(2,6-dichlorophenyl)-4-((4- (piperidine-1- carbonyl)phenyl)amino)-1H-pyrazole- 3-carboxamide |
| 83 | 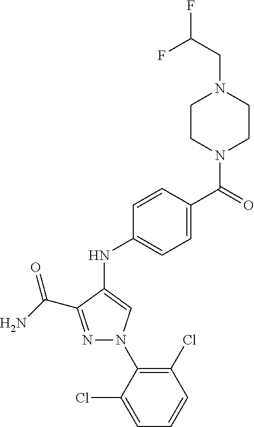 [SMILES: NC(=O)c1nn(-c2c(Cl)cccc2Cl)cc1Nc1ccc(C(=O)N2CCN(CC(F)F)CC2)cc1] | 1-(2,6-dichlorophenyl)-4-((4-(4-(2,2- difluoroethyl)piperazine-1- carbonyl)phenyl)amino)-1H-pyrazole- 3-carboxamide |
| 84 | 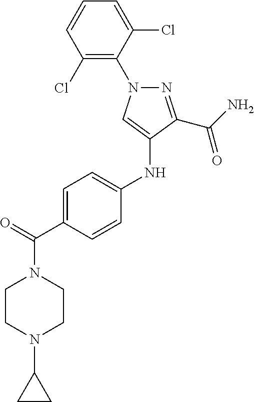 [SMILES: NC(=O)c1nn(-c2c(Cl)cccc2Cl)cc1Nc1ccc(C(=O)N2CCN(C3CC3)CC2)cc1] | 4-((4-(4-cyclopropylpiperazine-1- carbonyl)phenyl)amino)-1-(2,6- dichlorophenyl)-1H-pyrazole-3- carboxamide |
| 85 | 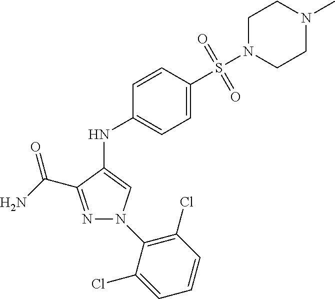 [SMILES: CN1CCN(S(=O)(=O)c2ccc(Nc3cn(-c4c(Cl)cccc4Cl)nc3C(N)=O)cc2)CC1] | 1-(2,6-dichlorophenyl)-4-((4-((4- methylpiperazin-1- yl)sulfonyl)phenyl)amino)-1H- pyrazole-3-carboxamide |
| 86 | 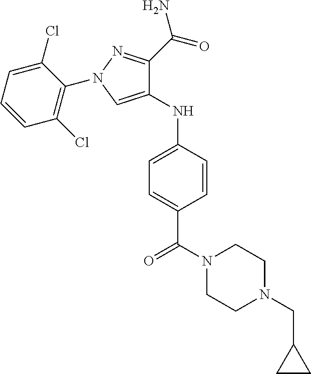 [SMILES: NC(=O)c1nn(-c2c(Cl)cccc2Cl)cc1Nc1ccc(C(=O)N2CCN(CC3CC3)CC2)cc1] | 4-((4-(4- (cyclopropylmethyl)piperazine-1- carbonyl)phenyl)amino)-1-(2,6- dichlorophenyl)-1H-pyrazole-3- carboxamide |
| 87 | 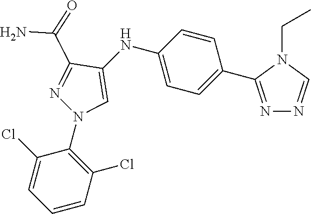 [SMILES: CCn1cnnc1-c1ccc(Nc2cn(-c3c(Cl)cccc3Cl)nc2C(N)=O)cc1] | 1-(2,6-dichlorophenyl)-4-((4-(4-ethyl- 4H-1,2,4-triazol-3-yl)phenyl)amino)- 1H-pyrazole-3-carboxamide |
| 88 | 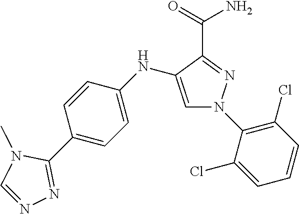 [SMILES: Cn1cnnc1-c1ccc(Nc2cn(-c3c(Cl)cccc3Cl)nc2C(N)=O)cc1] | 1-(2,6-dichlorophenyl)-4-((4-(4- methyl-4H-1,2,4-triazol-3- yl)phenyl)amino)-1H-pyrazole-3- carboxamide |
| 89 | 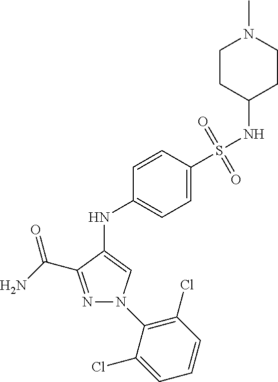 [SMILES: CN1CCC(NS(=O)(=O)c2ccc(Nc3cn(-c4c(Cl)cccc4Cl)nc3C(N)=O)cc2)CC1] | 1-(2,6-dichlorophenyl)-4-((4-(N-(1- methylpiperidin-4- yl)sulfamoyl)phenyl)amino)-1H- pyrazole-3-carboxamide |
| 90 | 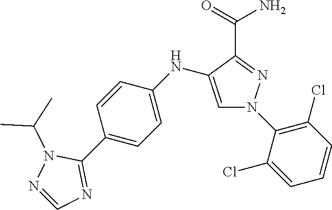 [SMILES: CC(C)n1ncnc1-c1ccc(Nc2cn(-c3c(Cl)cccc3Cl)nc2C(N)=O)cc1] | 1-(2,6-dichlorophenyl)-4-((4-(1- isopropyl-1H-1,2,4-triazol-5- yl)phenyl)amino)-1H-pyrazole-3- carboxamide |
| 91 | 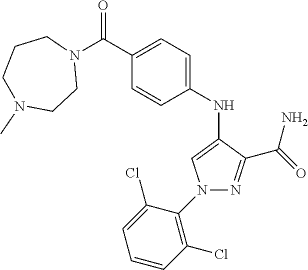 [SMILES: CN1CCCN(C(=O)c2ccc(Nc3cn(-c4c(Cl)cccc4Cl)nc3C(N)=O)cc2)CC1] | 1-(2,6-dichlorophenyl)-4-((4-(4- methyl-1,4-diazepane-1- carbonyl)phenyl)amino)-1H-pyrazole- 3-carboxamide |
| 92 | 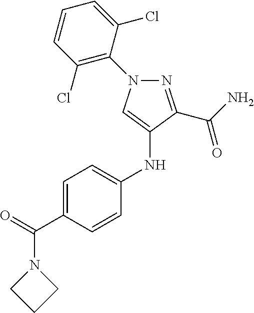 [SMILES: NC(=O)c1nn(-c2c(Cl)cccc2Cl)cc1Nc1ccc(C(=O)N2CCC2)cc1] | 4-((4-(azetidine-1- carbonyl)phenyl)amino)-1-(2,6- dichlorophenyl)-1H-pyrazole-3- carboxamide |
| 93 | 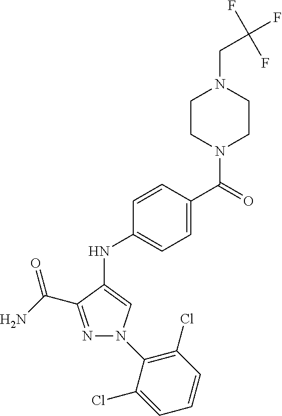 [SMILES: NC(=O)c1nn(-c2c(Cl)cccc2Cl)cc1Nc1ccc(C(=O)N2CCN(CC(F)(F)F)CC2)cc1] | 1-(2,6-dichlorophenyl)-4-((4-(4- (2,2,2-trifluoroethyl)piperazine-1- carbonyl)phenyl)amino)-1H-pyrazole- 3-carboxamide |
| 94 | 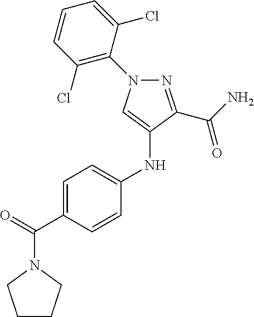 [SMILES: NC(=O)c1nn(-c2c(Cl)cccc2Cl)cc1Nc1ccc(C(=O)N2CCCC2)cc1] | 1-(2,6-dichlorophenyl)-4-((4- (pyrrolidine-1- carbonyl)phenyl)amino)-1H-pyrazole- 3-carboxamide |
| 95 | 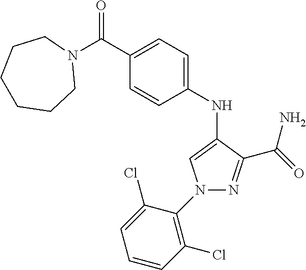 [SMILES: NC(=O)c1nn(-c2c(Cl)cccc2Cl)cc1Nc1ccc(C(=O)N2CCCCCC2)cc1] | 4-((4-(azepane-1- carbonyl)phenyl)amino)-1-(2,6- dichlorophenyl)-1H-pyrazole-3- carboxamide |
| 96 | 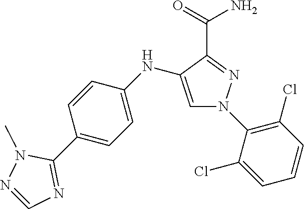 [SMILES: Cn1ncnc1-c1ccc(Nc2cn(-c3c(Cl)cccc3Cl)nc2C(N)=O)cc1] | 1-(2,6-dichlorophenyl)-4-((4-(1- methyl-1H-1,2,4-triazol-5- yl)phenyl)amino)-1H-pyrazole-3- carboxamide |
| 201 | 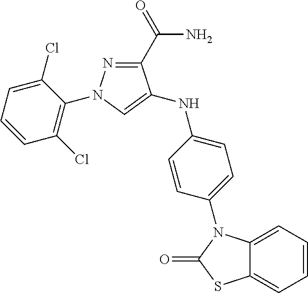 [SMILES: NC(=O)c1nn(-c2c(Cl)cccc2Cl)cc1Nc1ccc(-n2c(=O)sc3ccccc32)cc1] | 1-(2,6-dichlorophenyl)-4-((4-(2- oxobenzo[d]thiazol-3(2H)- yl)phenyl)amino)-1H-pyrazole-3- carboxamide |
| 202 | 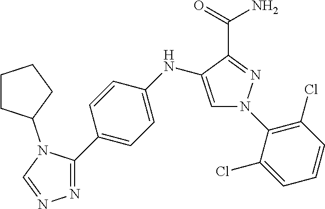 [SMILES: NC(=O)c1nn(-c2c(Cl)cccc2Cl)cc1Nc1ccc(-c2nncn2C2CCCC2)cc1] | 4-((4-(4-cyclopentyl-4H-1,2,4-triazol- 3-yl)phenyl)amino)-1-(2,6- dichlorophenyl)-1H-pyrazole-3- carboxamide |
| 203 | 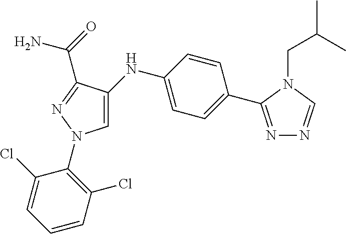 [SMILES: CC(C)Cn1cnnc1-c1ccc(Nc2cn(-c3c(Cl)cccc3Cl)nc2C(N)=O)cc1] | 1-(2,6-dichlorophenyl)-4-((4-(4- isobutyl-4H-1,2,4-triazol-3- yl)phenyl)amino)-1H-pyrazole-3- carboxamide |
| 204 | 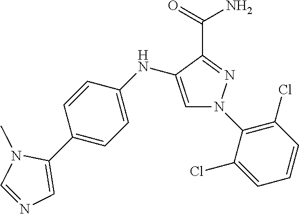 [SMILES: Cn1cncc1-c1ccc(Nc2cn(-c3c(Cl)cccc3Cl)nc2C(N)=O)cc1] | 1-(2,6-dichlorophenyl)-4-((4-(1- methyl-1H-imidazol-5- yl)phenyl)amino)-1H-pyrazole-3- carboxamide |
| 205 | 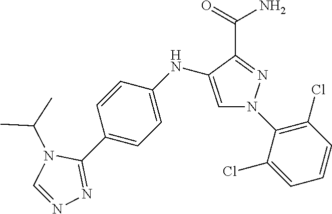 [SMILES: CC(C)n1cnnc1-c1ccc(Nc2cn(-c3c(Cl)cccc3Cl)nc2C(N)=O)cc1] | 1-(2,6-dichlorophenyl)-4-((4-(4- isopropyl-4H-1,2,4-triazol-3- yl)phenyl)amino)-1H-pyrazole-3- carboxamide |
| 206 | 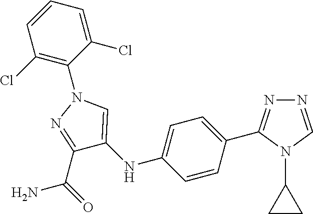 [SMILES: NC(=O)c1nn(-c2c(Cl)cccc2Cl)cc1Nc1ccc(-c2nncn2C2CC2)cc1] | 4-((4-(4-cyclopropyl-4H-1,2,4-triazol- 3-yl)phenyl)amino)-1-(2,6- dichlorophenyl)-1H-pyrazole-3- carboxamide |
| 207 | 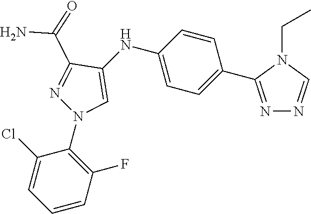 [SMILES: CCn1cnnc1-c1ccc(Nc2cn(-c3c(F)cccc3Cl)nc2C(N)=O)cc1] | 1-(2-chloro-6-fluorophenyl)-4-((4-(4- ethyl-4H-1,2,4-triazol-3- yl)phenyl)amino)-1H-pyrazole-3- carboxamide |
| 208 | 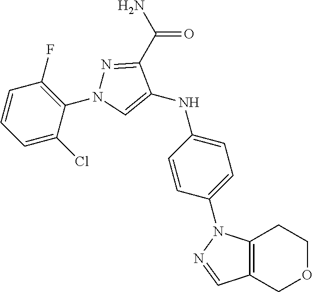 [SMILES: NC(=O)c1nn(-c2c(F)cccc2Cl)cc1Nc1ccc(-n2ncc3c2CCOC3)cc1] | 1-(2-chloro-6-fluorophenyll)-4-((4- (6,7-dihydropyrano[4,3-c]pyrazol- 1(4H)-yl)phenyl)amino)-1H-pyrazole- 3-carboxamide |
| 209 | 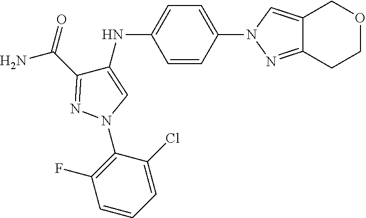 [SMILES: NC(=O)c1nn(-c2c(F)cccc2Cl)cc1Nc1ccc(-n2cc3c(n2)CCOC3)cc1] | 1-(2-chloro-6-fluorophenyl)-4-((4- (6,7-dihydropyrano[4,3-c]pyrazol- 2(4H)-yl)phenyl)amino)-1H-pyrazole- 3-carboxamide |
| 210 | 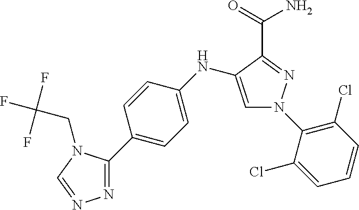 [SMILES: NC(=O)c1nn(-c2c(Cl)cccc2Cl)cc1Nc1ccc(-c2nncn2CC(F)(F)F)cc1] | 1-(2,6-dichlorophenyl)-4-((4-(4- (2,2,2-trifluoroethyl)-4H-1,2,4-triazol- 3-yl)phenyl)amino)-1H-pyrazole-3- carboxamide |
| 211 | 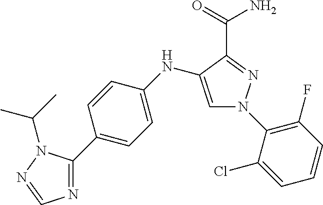 [SMILES: CC(C)n1ncnc1-c1ccc(Nc2cn(-c3c(F)cccc3Cl)nc2C(N)=O)cc1] | 1-(2-chloro-6-fluorophenyl)-4-((4-(1- isopropyl-1H-1,2,4-triazol-5- yl)phenyl)amino)-1H-pyrazole-3- carboxamide |
| 212 | 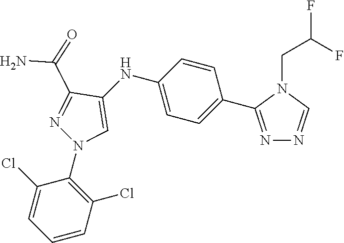 [SMILES: NC(=O)c1nn(-c2c(Cl)cccc2Cl)cc1Nc1ccc(-c2nncn2CC(F)F)cc1] | 1-(2,6-dichlorophenyl)-4-((4-(4-(2,2- difluoroethyl)-4H-1,2,4-triazol-3- yl)phenyl)amino)-1H-pyrazole-3- carboxamide |
| 213 | 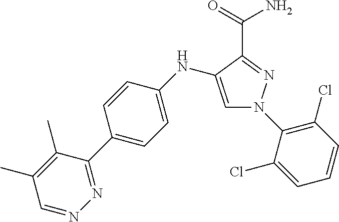 [SMILES: Cc1cnnc(-c2ccc(Nc3cn(-c4c(Cl)cccc4Cl)nc3C(N)=O)cc2)c1C] | 1-(2,6-dichlorophenyl)-4-((4-(4,5- dimethylpyridazin-3- yl)phenyl)amino)-1H-pyrazole-3- carboxamide |
| 214 | 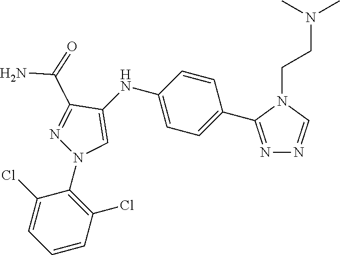 [SMILES: CN(C)CCn1cnnc1-c1ccc(Nc2cn(-c3c(Cl)cccc3Cl)nc2C(N)=O)cc1] | 1-(2,6-dichlorophenyl)-4-((4-(4-(2- (dimethylamino)ethyl)-4H-1,2,4- triazol-3-yl)phenyl)amino)-1H- pyrazole-3-carboxamide |
| 215 | 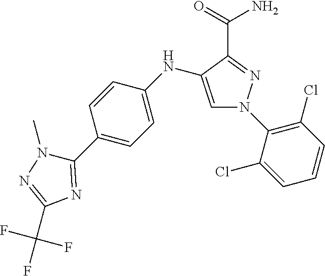 [SMILES: Cn1nc(C(F)(F)F)nc1-c1ccc(Nc2cn(-c3c(Cl)cccc3Cl)nc2C(N)=O)cc1] | 1-(2,6-dichlorophenyl)-4-((4-(1- methyl-3-(trifluoromethyl)-1H-1,2,4- triazol-5-yl)phenyl)amino)-1H- pyrazole-3-carboxamide |
| 216 | 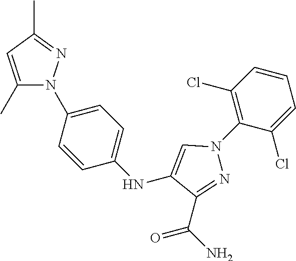 [SMILES: Cc1cc(C)n(-c2ccc(Nc3cn(-c4c(Cl)cccc4Cl)nc3C(N)=O)cc2)n1] | 1-(2,6-dichlorophenyl)-4-((4-(3,5- dimethyl-1H-pyrazol-1- yl)phenyl)amino)-1H-pyrazole-3- carboxamide |
| 217 | 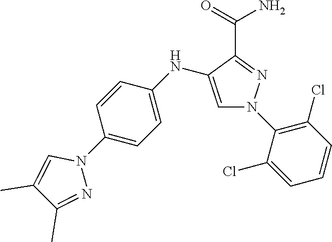 [SMILES: Cc1cn(-c2ccc(Nc3cn(-c4c(Cl)cccc4Cl)nc3C(N)=O)cc2)nc1C] | 1-(2,6-dichlorophenyl)-4-((4-(3,4- dimethyl-1H-pyrazol-1- yl)phenyl)amino)-1H-pyrazole-3- carboxamide |
| 218 | 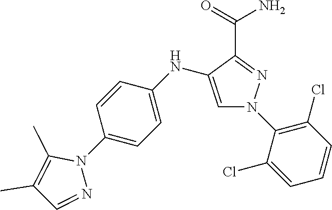 [SMILES: Cc1cnn(-c2ccc(Nc3cn(-c4c(Cl)cccc4Cl)nc3C(N)=O)cc2)c1C] | 1-(2,6-dichlorophenyl)-4-((4-(4,5- dimethyl-1H-pyrazol-1- yl)phenyl)amino)-1H-pyrazole-3- carboxamide |
| 219 | 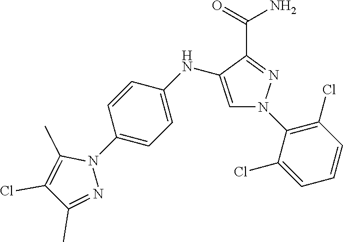 [SMILES: Cc1nn(-c2ccc(Nc3cn(-c4c(Cl)cccc4Cl)nc3C(N)=O)cc2)c(C)c1Cl] | 4-((4-(4-chloro-3,5-dimethyl-1H- pyrazol-1-yl)phenyl)amino)-1-(2,6- dichlorophenyl)-1H-pyrazole-3- carboxamide |
| 220 | 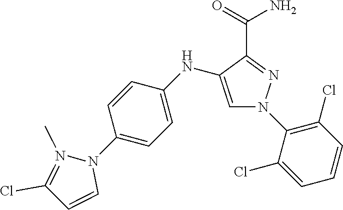 | 4-((4-(4-chloro-5-methyl-1H-pyrazol- 1-yl)phenyl)amino)-1-(2,6- dichlorophenyl)-1H-pyrazole-3- carboxamide |
| 221 | 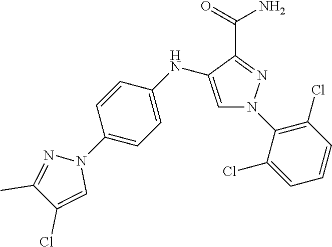 [SMILES: Cc1nn(-c2ccc(Nc3cn(-c4c(Cl)cccc4Cl)nc3C(N)=O)cc2)cc1Cl] | 4-((4-(4-chloro-3-methyl-1H-pyrazol- 1-yl)phenyl)amino)-1-(2,6- dichlorophenyl)-1H-pyrazole-3- carboxamide |
| 222 | 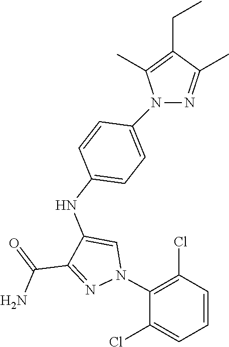 [SMILES: CCc1c(C)nn(-c2ccc(Nc3cn(-c4c(Cl)cccc4Cl)nc3C(N)=O)cc2)c1C] | 1-(2,6-dichlorophenyl)-4-((4-(4-ethyl- 3,5-dimethyl-1H-pyrazol-1- yl)phenyl)amino)-1H-pyrazole-3- carboxamide |
| 223 | 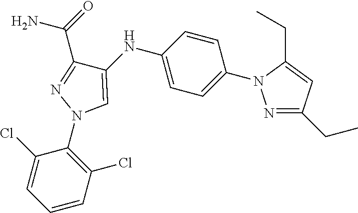 [SMILES: CCc1cc(CC)n(-c2ccc(Nc3cn(-c4c(Cl)cccc4Cl)nc3C(N)=O)cc2)n1] | 1-(2,6-dichlorophenyl)-4-((4-(3,5- diethyl-1H-pyrazol-1- yl)phenyl)amino)-1H-pyrazole-3- carboxamide |
| 224 | 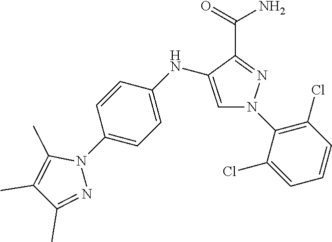 [SMILES: Cc1nn(-c2ccc(Nc3cn(-c4c(Cl)cccc4Cl)nc3C(N)=O)cc2)c(C)c1C] | 1-(2,6-dichlorophenyl)-4-((4-(3,4,5- trimethyl-1H-pyrazol-1- yl)phenyl)amino)-1H-pyrazole-3- carboxamide |
| 225 | 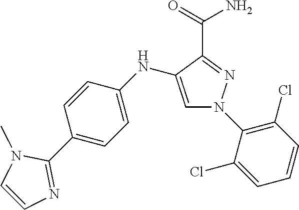 [SMILES: Cn1ccnc1-c1ccc(Nc2cn(-c3c(Cl)cccc3Cl)nc2C(N)=O)cc1] | 1-(2,6-dichlorophenyl)-4-((4-(1- methyl-1H-imidazol-2- yl)phenyl)amino)-1H-pyrazole-3- carboxamide |
| 226 | 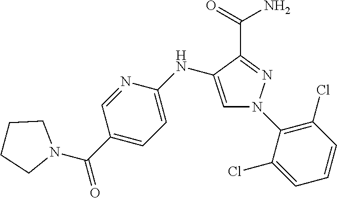 [SMILES: NC(=O)c1nn(-c2c(Cl)cccc2Cl)cc1Nc1ccc(C(=O)N2CCCC2)cn1] | 1-(2,6-dichlorophenyl)-4-((5- (pyrrolidine-1-carbonyl)pyridin-2- yl)amino)-1H-pyrazole-3- carboxamide |
| 227 | 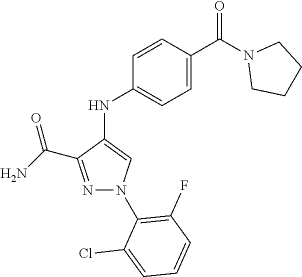 [SMILES: NC(=O)c1nn(-c2c(F)cccc2Cl)cc1Nc1ccc(C(=O)N2CCCC2)cc1] | 1-(2-chloro-6-fluorophenyl)-4-((4- (pyrrolidine-1- carbonyl)phenyl)amino)-1H-pyrazole- 3-carboxamide |
| 228 | 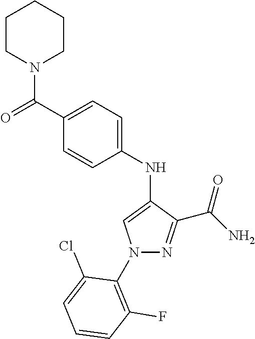 [SMILES: NC(=O)c1nn(-c2c(F)cccc2Cl)cc1Nc1ccc(C(=O)N2CCCCC2)cc1] | 1-(2-chloro-6-fluorophenyl)-4-((4- (piperidine-1- carbonyl)phenyl)amino)-1H-pyrazole- 3-carboxamide |
| 229 | 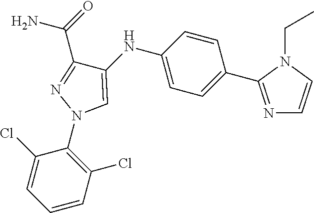 [SMILES: CCn1ccnc1-c1ccc(Nc2cn(-c3c(Cl)cccc3Cl)nc2C(N)=O)cc1] | 1-(2,6-dichlorophenyl)-4-((4-(1-ethyl- 1H-imidazol-2-yl)phenyl)amino)-1H- pyrazole-3-carboxamide |
| 230 | 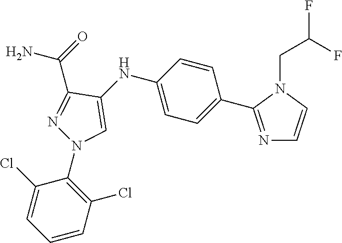 [SMILES: NC(=O)c1nn(-c2c(Cl)cccc2Cl)cc1Nc1ccc(-c2nccn2CC(F)F)cc1] | 1-(2,6-dichlorophenyl)-4-((4-(1-(2,2- difluoroethyl)-1H-imidazol-2- yl)phenyl)amino)-1H-pyrazole-3- carboxamide |
| 231 | 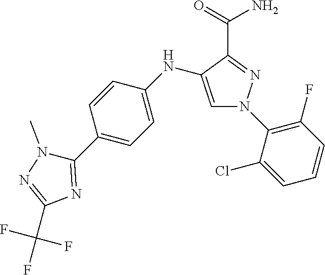 [SMILES: Cn1nc(C(F)(F)F)nc1-c1ccc(Nc2cn(-c3c(F)cccc3Cl)nc2C(N)=O)cc1] | 1-(2-chloro-6-fluorophenyl)-4-((4-(1- methyl-3-(trifluoromethyl)-1H-1,2,4- triazol-5-yl)phenyl)amino)-1H- pyrazole-3-carboxamide |
| 232 | 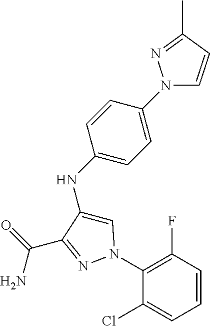 [SMILES: Cc1ccn(-c2ccc(Nc3cn(-c4c(F)cccc4Cl)nc3C(N)=O)cc2)n1] | 1-(2-chloro-6-fluorophenyl)-4-((4-(3- methyl-1H-pyrazol-1- yl)phenyl)amino)-1H-pyrazole-3- carboxamide |
| 233 | 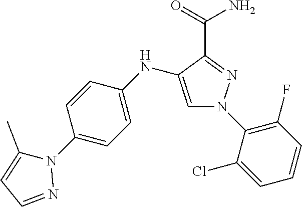 [SMILES: Cc1ccnn1-c1ccc(Nc2cn(-c3c(F)cccc3Cl)nc2C(N)=O)cc1] | 1-(2-chloro-6-fluorophenyl)-4-((4-(5- methyl-1H-pyrazol-1- yl)phenyl)amino)-1H-pyrazole-3- carboxamide |
| 234 | 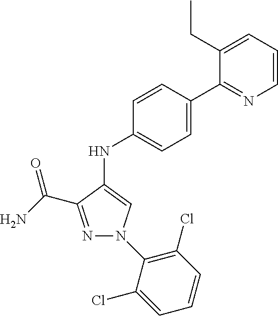 [SMILES: CCc1cccnc1-c1ccc(Nc2cn(-c3c(Cl)cccc3Cl)nc2C(N)=O)cc1] | 1-(2,6-dichlorophenyl)-4-((4-(3- ethylpyridin-2-yl)phenyl)amino)-1H- pyrazole-3-carboxamide |
| 235 | 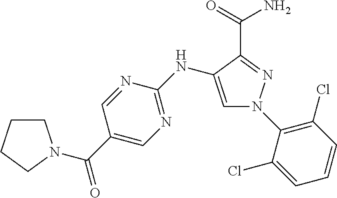 [SMILES: NC(=O)c1nn(-c2c(Cl)cccc2Cl)cc1Nc1ncc(C(=O)N2CCCC2)cn1] | 1-(2,6-dichlorophenyl)-4-((5- (pyrrolidine-1-carbonyl)pyrimidin-2- yl)amino)-1H-pyrazole-3- carboxamide |
| 236 | 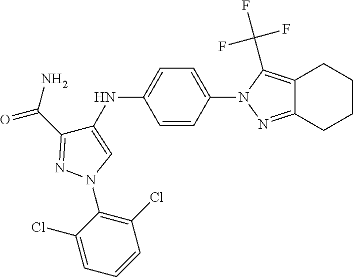 [SMILES: NC(=O)c1nn(-c2c(Cl)cccc2Cl)cc1Nc1ccc(-n2nc3c(c2C(F)(F)F)CCCC3)cc1] | 1-(2,6-dichlorophenyl)-4-((4-(3- (trifluoromethyl)-4,5,6,7-tetrahydro- 2H-indazol-2-yl)phenyl)amino)-1H- pyrazole-3-carboxamide |
| 237 | 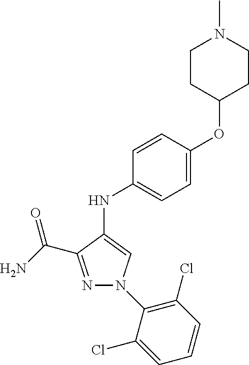 [SMILES: CN1CCC(Oc2ccc(Nc3cn(-c4c(Cl)cccc4Cl)nc3C(N)=O)cc2)CC1] | 1-(2,6-dichlorophenyl)-4-((4-((1- methylpiperidin-4- yl)oxy)phenyl)amino)-1H-pyrazole-3- carboxamide |
| 238 | 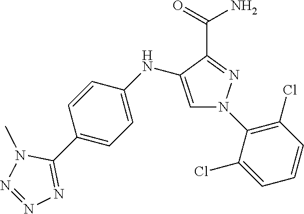 [SMILES: Cn1nnnc1-c1ccc(Nc2cn(-c3c(Cl)cccc3Cl)nc2C(N)=O)cc1] | 1-(2,6-dichlorophenyl)-4-((4-(1- methyl-1H-tetrazol-5- yl)phenyl)amino)-1H-pyrazole-3- carboxamide |
| 239 | 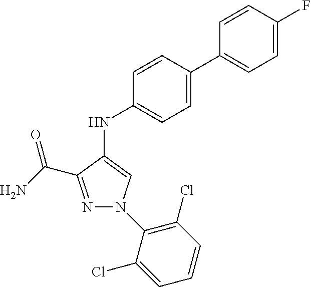 [SMILES: NC(=O)c1nn(-c2c(Cl)cccc2Cl)cc1Nc1ccc(-c2ccc(F)cc2)cc1] | 1-(2,6-dichlorophenyl)-4-((4′-fluoro- [1,1′-biphenyl]-4-yl)amino)-1H- pyrazole-3-carboxamide |
| 240 | 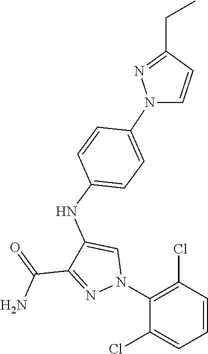 [SMILES: CCc1ccn(-c2ccc(Nc3cn(-c4c(Cl)cccc4Cl)nc3C(N)=O)cc2)n1] | 1-(2,6-dichlorophenyl)-4-((4-(3-ethyl- 1H-pyrazol-1-yl)phenyl)amino)-1H- pyrazole-3-carboxamide |
| 241 | 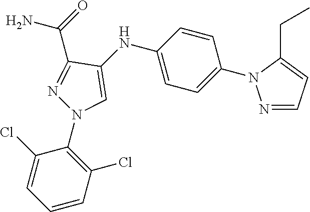 [SMILES: CCc1ccnn1-c1ccc(Nc2cn(-c3c(Cl)cccc3Cl)nc2C(N)=O)cc1] | 1-(2,6-dichlorophenyl)-4-((4-(5-ethyl- 1H-pyrazol-1-yl)phenyl)amino)-1H- pyrazole-3-carboxamide |
| 242 | 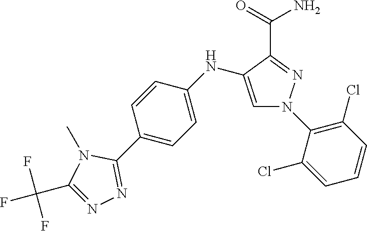 [SMILES: Cn1c(-c2ccc(Nc3cn(-c4c(Cl)cccc4Cl)nc3C(N)=O)cc2)nnc1C(F)(F)F] | 1-(2,6-dichlorophenyl)-4-((4-(4- methyl-5-(trifluoromethyl)-4H-1,2,4- triazol-3-yl)phenyl)amino)-1H- pyrazole-3-carboxamide |
| 243 | 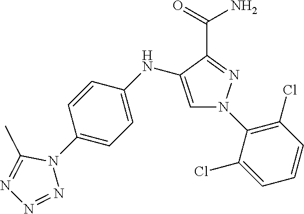 [SMILES: Cc1nnnn1-c1ccc(Nc2cn(-c3c(Cl)cccc3Cl)nc2C(N)=O)cc1] | 1-(2,6-dichlorophenyl)-4-((4-(5- methyl-1H-tetrazol-1- yl)phenyl)amino)-1H-pyrazole-3- carboxamide |
| 244 | 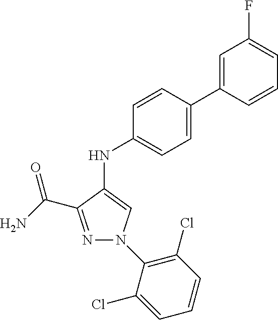 [SMILES: NC(=O)c1nn(-c2c(Cl)cccc2Cl)cc1Nc1ccc(-c2cccc(F)c2)cc1] | 1-(2,6-dichlorophenyl)-4-((3′-fluoro- [1,1′-biphenyl]-4-yl)amino)-1H- pyrazole-3-carboxamide |
| 245 | 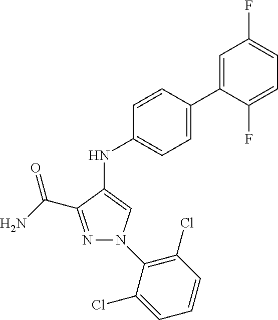 [SMILES: NC(=O)c1nn(-c2c(Cl)cccc2Cl)cc1Nc1ccc(-c2cc(F)ccc2F)cc1] | 1-(2,6-dichlorophenyl)-4-((2′,5′- difluoro-[1,1′-biphenyl]-4-yl)amino)- 1H-pyrazole-3-carboxamide |
| 246 | 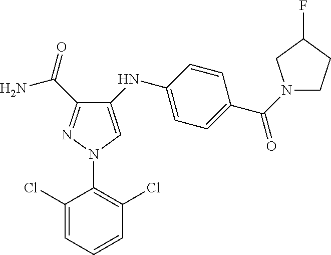 [SMILES: NC(=O)c1nn(-c2c(Cl)cccc2Cl)cc1Nc1ccc(C(=O)N2CCC(F)C2)cc1] | 1-(2,6-dichlorophenyl)-4-((4-(3- fluoropyrrolidine-1- carbonyl)phenyl)amino)-1H-pyrazole- 3-carboxamide |
| 247 | 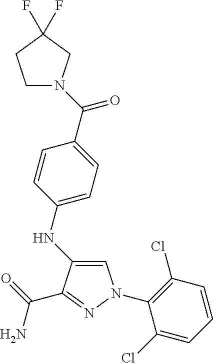 [SMILES: NC(=O)c1nn(-c2c(Cl)cccc2Cl)cc1Nc1ccc(C(=O)N2CCC(F)(F)C2)cc1] | 1-(2,6-dichlorophenyl)-4-((4-(3,3- difluoropyrrolidine-1- carbonyl)phenyl)amino)-1H-pyrazole- 3-carboxamide |
| 248 | 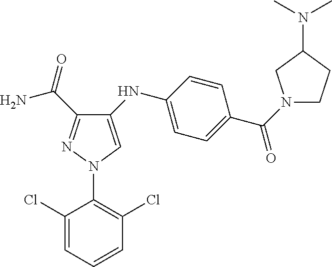 [SMILES: CN(C)C1CCN(C(=O)c2ccc(Nc3cn(-c4c(Cl)cccc4Cl)nc3C(N)=O)cc2)C1] | 1-(2,6-dichlorophenyl)-4-((4-(3- (dimethylamino)pyrrolidine-1- carbonyl)phenyl)amino)-1H-pyrazole- 3-carboxamide |
| 249 | 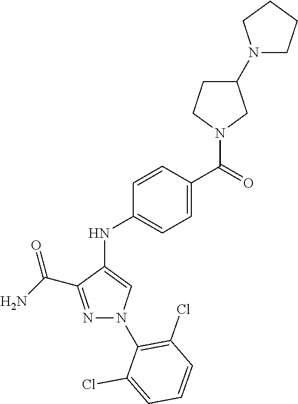 [SMILES: NC(=O)c1nn(-c2c(Cl)cccc2Cl)cc1Nc1ccc(C(=O)N2CCC(N3CCCC3)C2)cc1] | 4-((4-([1,3′-bipyrrolidine]-1′- carbonyl)phenyl)amino)-1-(2,6- dichlorophenyl)-1H-pyrazole-3- carboxamide |
| 250 | 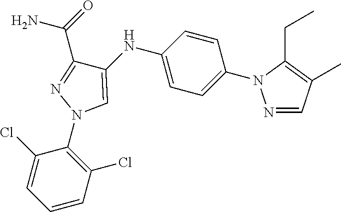 [SMILES: CCc1c(C)cnn1-c1ccc(Nc2cn(-c3c(Cl)cccc3Cl)nc2C(N)=O)cc1] | 1-(2,6-dichlorophenyl)-4-((4-(5-ethyl- 4-methyl-1H-pyrazol-1- yl)phenyl)amino)-1H-pyrazole-3- carboxamide |
| 251 | 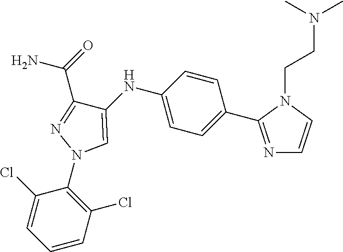 [SMILES: CN(C)CCn1ccnc1-c1ccc(Nc2cn(-c3c(Cl)cccc3Cl)nc2C(N)=O)cc1] | 1-(2,6-dichlorophenyl)-4-((4-(1-(2- (dimethylamino)ethyl)-1H-imidazol- 2-yl)phenyl)amino)-1H-pyrazole-3- carboxamide |
| 252 | 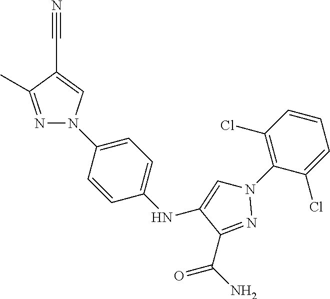 [SMILES: Cc1nn(-c2ccc(Nc3cn(-c4c(Cl)cccc4Cl)nc3C(N)=O)cc2)cc1C#N] | 4-((4-(4-cyano-3-methyl-1H-pyrazol- 1-yl)phenyl)amino)-1-(2,6- dichlorophenyl)-1H-pyrazole-3- carboxamide |
| 253 | 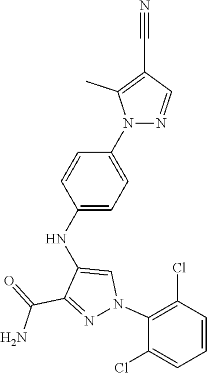 [SMILES: Cc1c(C#N)cnn1-c1ccc(Nc2cn(-c3c(Cl)cccc3Cl)nc2C(N)=O)cc1] | 4-((4-(4-cyano-5-methyl-1H-pyrazol- 1-yl)phenyl)amino)-1-(2,6- dichlorophenyl)-1H-pyrazole-3- carboxamide |
| 254 | 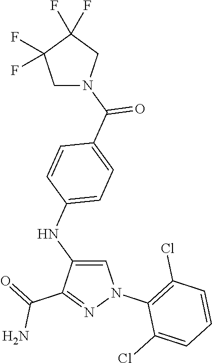 [SMILES: NC(=O)c1nn(-c2c(Cl)cccc2Cl)cc1Nc1ccc(C(=O)N2CC(F)(F)C(F)(F)C2)cc1] | 1-(2,6-dichlorophenyl)-4-((4-(3,3,4,4- tetrafluoropyrrolidine-1- carbonyl)phenyl)amino)-1H-pyrazole- 3-carboxamide |
| 255 | 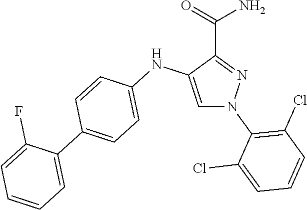 [SMILES: NC(=O)c1nn(-c2c(Cl)cccc2Cl)cc1Nc1ccc(-c2ccccc2F)cc1] | 1-(2,6-dichlorophenyl)-4-((2′-fluoro- [1,1′-biphenyl]-4-yl)amino)-1H- pyrazole-3-carboxamide |
| 256 | 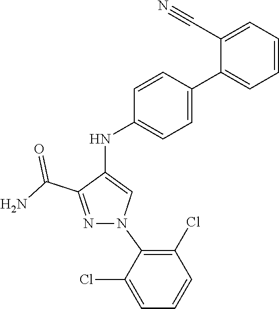 [SMILES: N#Cc1ccccc1-c1ccc(Nc2cn(-c3c(Cl)cccc3Cl)nc2C(N)=O)cc1] | 4-((2′-cyano-[1,1′-biphenyl]-4- yl)amino)-1-(2,6-dichlorophenyl)-1H- pyrazole-3-carboxamide |
| 257 | 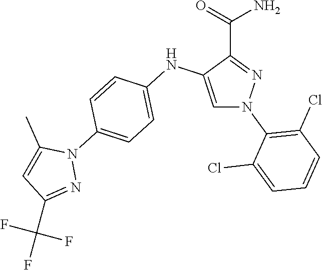 [SMILES: Cc1cc(C(F)(F)F)nn1-c1ccc(Nc2cn(-c3c(Cl)cccc3Cl)nc2C(N)=O)cc1] | 1-(2,6-dichlorophenyl)-4-((4-(5- methyl-3-(trifluoromethyl)-1H- pyrazol-1-yl)phenyl)amino)-1H- pyrazole-3-carboxamide |
| 258 | 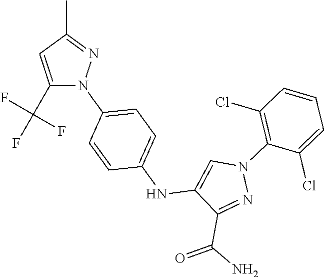 [SMILES: Cc1cc(C(F)(F)F)n(-c2ccc(Nc3cn(-c4c(Cl)cccc4Cl)nc3C(N)=O)cc2)n1] | 1-(2,6-dichlorophenyl)-4-((4-(3- methyl-5-(trifluoromethyl)-1H- pyrazol-1-yl)phenyl)amino)-1H- pyrazole-3-carboxamide |
| 259 | 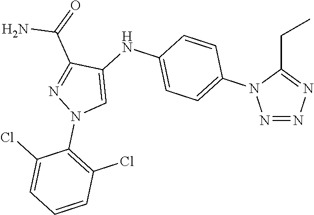 [SMILES: CCc1nnnn1-c1ccc(Nc2cn(-c3c(Cl)cccc3Cl)nc2C(N)=O)cc1] | 1-(2,6-dichlorophenyl)-4-((4-(5-ethyl- 1H-tetrazol-1-yl)phenyl)amino)-1H- pyrazole-3-carboxamide |
| 260 | 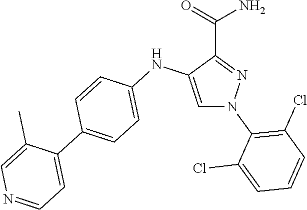 [SMILES: Cc1cnccc1-c1ccc(Nc2cn(-c3c(Cl)cccc3Cl)nc2C(N)=O)cc1] | 1-(2,6-dichlorophenyl)-4-((4-(3- methylpyridin-4-yl)phenyl)amino)- 1H-pyrazole-3-carboxamide |
| 261 | 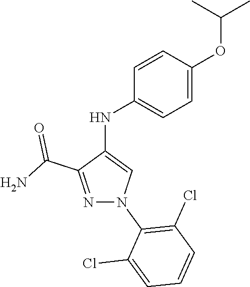 [SMILES: CC(C)Oc1ccc(Nc2cn(-c3c(Cl)cccc3Cl)nc2C(N)=O)cc1] | 1-(2,6-dichlorophenyl)-4-((4- isopropoxyphenyl)amino)-1H- pyrazole-3-carboxamide |
| 262 | 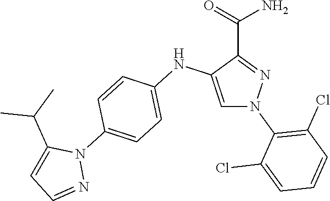 [SMILES: CC(C)c1ccnn1-c1ccc(Nc2cn(-c3c(Cl)cccc3Cl)nc2C(N)=O)cc1] | 1-(2,6-dichlorophenyl)-4-((4-(5- isopropyl-1H-pyrazol-1- yl)phenyl)amino)-1H-pyrazole-3- carboxamide |
| 263 | 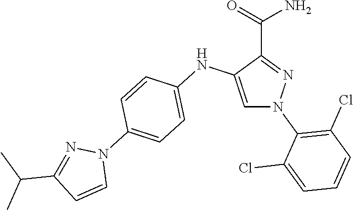 [SMILES: CC(C)c1ccn(-c2ccc(Nc3cn(-c4c(Cl)cccc4Cl)nc3C(N)=O)cc2)n1] | 1-(2,6-dichlorophenyl)-4-((4-(3- isopropyl-1H-pyrazol-1- yl)phenyl)amino)-1H-pyrazole-3- carboxamide |
| 264 | 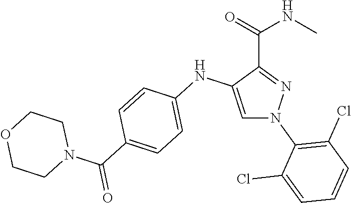 [SMILES: CNC(=O)c1nn(-c2c(Cl)cccc2Cl)cc1Nc1ccc(C(=O)N2CCOCC2)cc1] | 1-(2,6-dichlorophenyl)-N-methyl-4- ((4-(morpholine-4- carbonyl)phenyl)amino)-1H-pyrazole- 3-carboxamide |
| 265 | 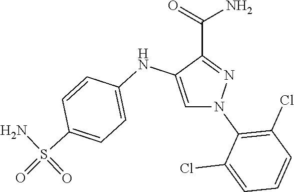 [SMILES: NC(=O)c1nn(-c2c(Cl)cccc2Cl)cc1Nc1ccc(S(N)(=O)=O)cc1] | 1-(2,6-dichlorophenyl)-4-((4- sulfamoylphenyl)amino)-1H- pyrazole-3-carboxamide |
| 266 | 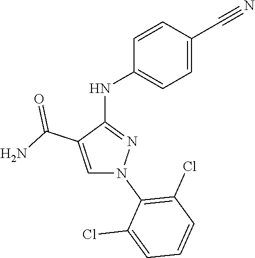 [SMILES: N#Cc1ccc(Nc2nn(-c3c(Cl)cccc3Cl)cc2C(N)=O)cc1] | 4-((4-cyanophenyl)amino)-1-(2,6- dichlorophenyl)-1H-pyrazole-3- carboxamide |
| 267 | 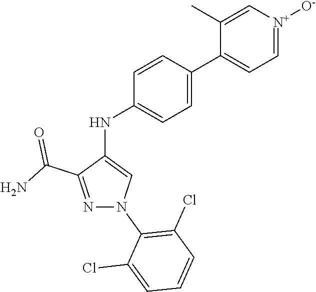 [SMILES: Cc1c[n+]([O-])ccc1-c1ccc(Nc2cn(-c3c(Cl)cccc3Cl)nc2C(N)=O)cc1] | 4-(4-((3-carbamoyl-1-(2,6- dichlorophenyl)-1H-pyrazol-4- yl)amino)phenyl)-3- methylpridine_1-oxide |
| 268 | 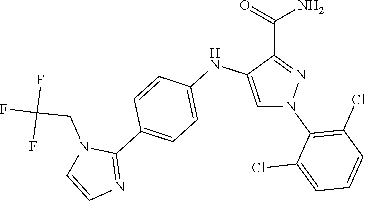 [SMILES: NC(=O)c1nn(-c2c(Cl)cccc2Cl)cc1Nc1ccc(-c2nccn2CC(F)(F)F)cc1] | 1-(2,6-dichlorophenyl)-4-((4-(1- (2,2,2-trifluoroethyl)-1H-imidazol-2- yl)phenyl)amino)-1H-pyrazole-3- carboxamide |
| 269 | 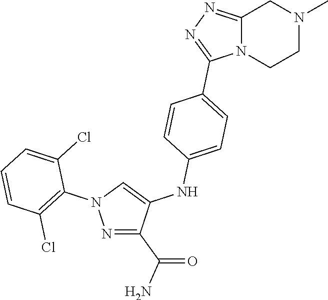 [SMILES: CN1CCn2c(nnc2-c2ccc(Nc3cn(-c4c(Cl)cccc4Cl)nc3C(N)=O)cc2)C1] | 1-(2,6-dichlorophenyl)-4-((4-(7- methyl-5,6,7,8-tetrahydro- [1,2,4]triazolo[4,3-a]pyrazin-3- yl)phenyl)amino)-1H-pyrazole-3- carboxamide |
| 270 | 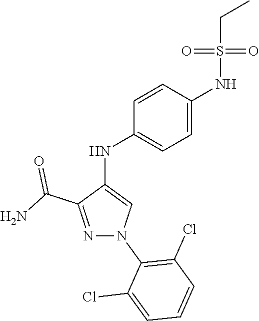 [SMILES: CCS(=O)(=O)Nc1ccc(Nc2cn(-c3c(Cl)cccc3Cl)nc2C(N)=O)cc1] | 1-(2,6-dichlorophenyl)-4-((4- (ethylsulfonamido)phenyl)amino)-1H- pyrazole-3-carboxamide |
| 271 | 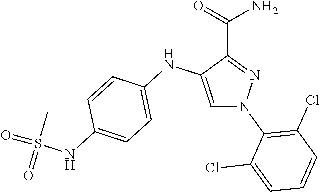 [SMILES: CS(=O)(=O)Nc1ccc(Nc2cn(-c3c(Cl)cccc3Cl)nc2C(N)=O)cc1] | 1-(2,6-dichlorophenyl)-4-((4- (methylsulfonamido)phenyl)amino)- 1H-pyrazole-3-carboxamide |
| 272 | 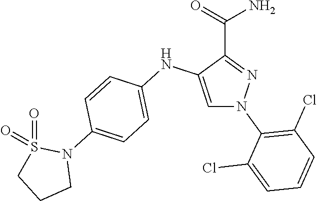 [SMILES: NC(=O)c1nn(-c2c(Cl)cccc2Cl)cc1Nc1ccc(N2CCCS2(=O)=O)cc1] | 1-(2,6-dichlorophenyl)-4-((4-(1,1- dioxidoisothiazolidin-2- yl)phenyl)amino)-1H-pyrazole-3- carboxamide |
| 273 | 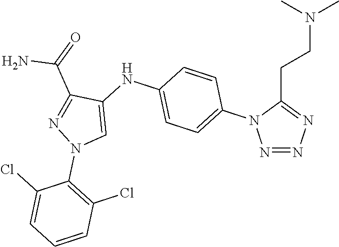 [SMILES: CN(C)CCc1nnnn1-c1ccc(Nc2cn(-c3c(Cl)cccc3Cl)nc2C(N)=O)cc1] | 1-(2,6-dichlorophenyl)-4-((4-(5-(2- (dimethylamino)ethyl)-1H-tetrazol-1- yl)phenyl)amino)-1H-pyrazole-3- carboxamide |
| 274 | 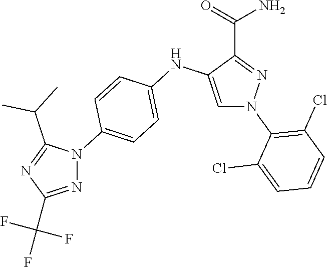 [SMILES: CC(C)c1nc(C(F)(F)F)nn1-c1ccc(Nc2cn(-c3c(Cl)cccc3Cl)nc2C(N)=O)cc1] | 1-(2,6-dichlorophenyl)-4-((4-(5- isopropyl-3-(trifluoromethyl)-1H- 1,2,4-triazol-1-yl)phenyl)amino)-1H- pyrazole-3-carboxamide |
| 275 | 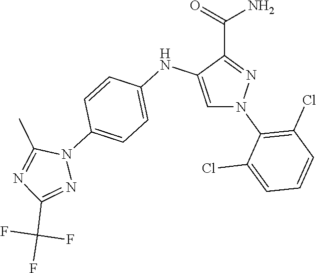 [SMILES: Cc1nc(C(F)(F)F)nn1-c1ccc(Nc2cn(-c3c(Cl)cccc3Cl)nc2C(N)=O)cc1] | 1-(2,6-dichlorophenyl)-4-((4-(5- methyl-3-(trifluoromethyl)-1H-1,2,4- triazol-1-yl)phenyl)amino)-1H- pyrazole-3-carboxamide |
| 276 | 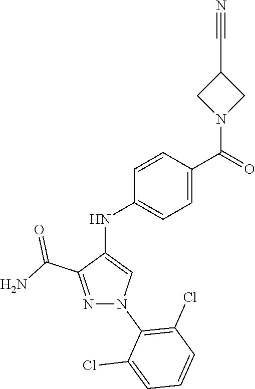 [SMILES: N#CC1CN(C(=O)c2ccc(Nc3cn(-c4c(Cl)cccc4Cl)nc3C(N)=O)cc2)C1] | 4-((4-(3-cyanoazetidine-1- carbonyl)phenyl)amino)-1-(2,6- dichlorophenyl)-1H-pyrazole-3- carboxamide |
| 277 | 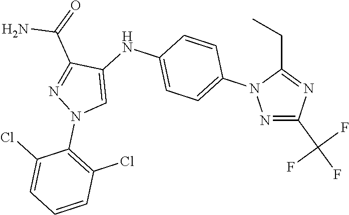 [SMILES: CCc1nc(C(F)(F)F)nn1-c1ccc(Nc2cn(-c3c(Cl)cccc3Cl)nc2C(N)=O)cc1] | 1-(2,6-dichlorophenyl)-4-((4-(5-ethyl- 3-(trifluoromethyl)-1H-1,2,4-triazol- 1-yl)phenyl)amino)-1H-pyrazole-3- carboxamide |
| 278 | 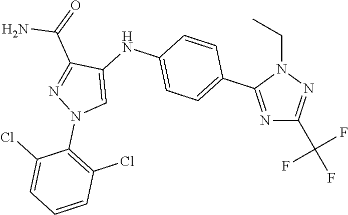 [SMILES: CCn1nc(C(F)(F)F)nc1-c1ccc(Nc2cn(-c3c(Cl)cccc3Cl)nc2C(N)=O)cc1] | 1-(2,6-dichlorophenyl)-4-((4-(1-ethyl- 3-(trifluoromethyl)-1H-1,2,4-triazol- 5-yl)phenyl)amino)-1H-pyrazole-3- carboxamide |
| 279 | 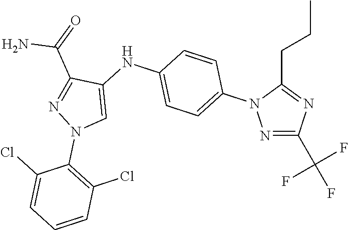 [SMILES: CCCc1nc(C(F)(F)F)nn1-c1ccc(Nc2cn(-c3c(Cl)cccc3Cl)nc2C(N)=O)cc1] | 1-(2,6-dichlorophenyl)-4-((4-(5- propyl-3-(trifluoromethyl)-1H-1,2,4- triazol-1-yl)phenyl)amino)-1H- pyrazole-3-carboxamide |
| 280 | 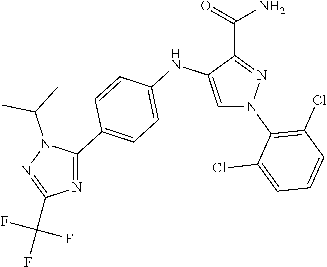 [SMILES: CC(C)n1nc(C(F)(F)F)nc1-c1ccc(Nc2cn(-c3c(Cl)cccc3Cl)nc2C(N)=O)cc1] | 1-(2,6-dichlorophenyl)-4-((4-(1- isopropyl-3-(trifluoromethyl)-1H- 1,2,4-triazol-5-yl)phenyl)amino)-1H- pyrazole-3-carboxamide |
| 281 | 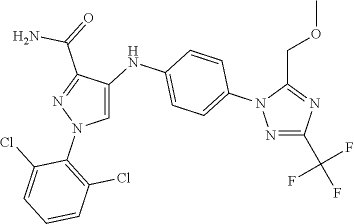 [SMILES: COCc1nc(C(F)(F)F)nn1-c1ccc(Nc2cn(-c3c(Cl)cccc3Cl)nc2C(N)=O)cc1] | 1-(2,6-dichlorophenyl)-4-((4-(5- (methoxymethyl)-3-(trifluoromethyl)- 1H-1,2,4-triazol-1-yl)phenyl)amino)- 1H-pyrazole-3-carboxamide |
| 282 | 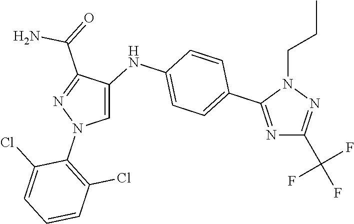 [SMILES: CCCn1nc(C(F)(F)F)nc1-c1ccc(Nc2cn(-c3c(Cl)cccc3Cl)nc2C(N)=O)cc1] | 1-(2,6-dichlorophenyl)-4-((4-(1- propyl-3-(trifluoromethyl)-1H-1,2,4- triazol-5-yl)phenyl)amino)-1H- pyrazole-3-carboxamide |
| 283 | 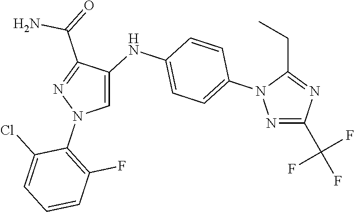 [SMILES: CCc1nc(C(F)(F)F)nn1-c1ccc(Nc2cn(-c3c(F)cccc3Cl)nc2C(N)=O)cc1] | 1-(2-chloro-6-fluorophenyl)-4-((4-(5- ethyl-3-(trifluoromethyl)-1H-1,2,4- triazol-1-yl)phenyl)amino)-1H- pyrazole-3-carboxamide |
| 284 | 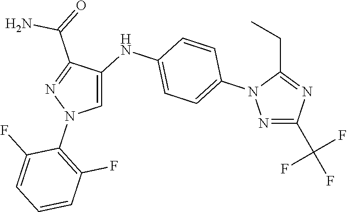 [SMILES: CCc1nc(C(F)(F)F)nn1-c1ccc(Nc2cn(-c3c(F)cccc3F)nc2C(N)=O)cc1] | 1-(2,6-dichlorophenyl)-4-((4-(5-ethyl- 3-(trifluoromethyl)-1H-1,2,4-triazol- 1-yl)phenyl)amino)-1H-pyrazole-3- carboxamide |
| 285 | 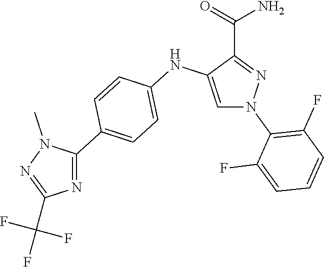 [SMILES: Cn1nc(C(F)(F)F)nc1-c1ccc(Nc2cn(-c3c(F)cccc3F)nc2C(N)=O)cc1] | 1-(2,6-difluorophenyl)-4-((4-(1- methyl-3-(trifluoromethyl)-1H-1,2,4- triazol-5-yl)phenyl)amino)-1H- pyrazole-3-carboxamide |
| 286 | 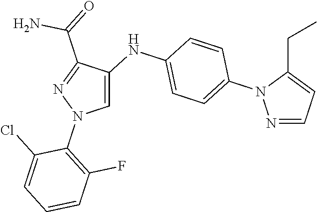 [SMILES: CCc1ccnn1-c1ccc(Nc2cn(-c3c(F)cccc3Cl)nc2C(N)=O)cc1] | 1-(2-chloro-6-fluorophenyl)-4-((4-(5- ethyl-1H-pyrazol-1-yl)phenyl)amino)- 1H-pyrazole-3-carboxamide |
| 287 | 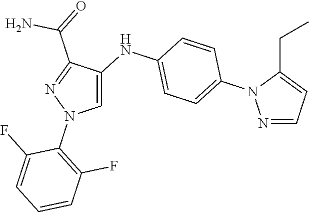 [SMILES: CCc1ccnn1-c1ccc(Nc2cn(-c3c(F)cccc3F)nc2C(N)=O)cc1] | 1-(2,6-difluorophenyl)-4-((4-(5-ethyl- 1H-pyrazol-1-yl)phenyl)amino)-1H- pyrazole-3-carboxamide |
| 288 | 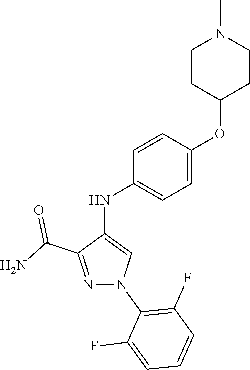 [SMILES: CN1CCC(Oc2ccc(Nc3cn(-c4c(F)cccc4F)nc3C(N)=O)cc2)CC1] | 1-(2,6-difluorophenyl)-4-((4-((1- methylpiperidin-4- yl)oxy)phenyl)amino)-1H-pyrazole-3- carboxamide |
| 289 | 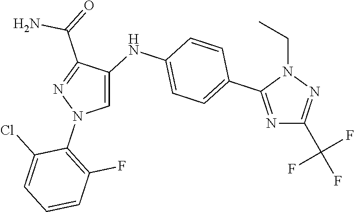 [SMILES: CCn1nc(C(F)(F)F)nc1-c1ccc(Nc2cn(-c3c(F)cccc3Cl)nc2C(N)=O)cc1] | 1-(2-chloro-6-fluorophenyl)-4-((4-(1- ethyl-3-(trifluoromethyl)-1H-1,2,4- triazol-5-yl)phenyl)amino)-1H- pyrazole-3-carboxamide |
| 290 | 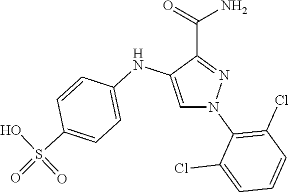 [SMILES: NC(=O)c1nn(-c2c(Cl)cccc2Cl)cc1Nc1ccc(S(=O)(=O)O)cc1] | 4-((3-carbamoyl-1-(2,6- dichlorophenyl)-1H-pyrazol-4- yl)amino)benzenesulfonic_acid |
| 291 | 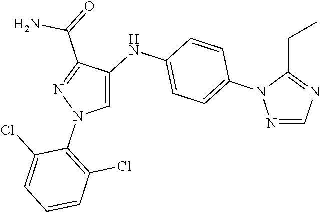 [SMILES: CCc1ncnn1-c1ccc(Nc2cn(-c3c(Cl)cccc3Cl)nc2C(N)=O)cc1] | 1-(2,6-dichlorophenyl)-4-((4-(5-ethyl- 1H-1,2,4-triazol-1-yl)phenyl)amino)- 1H-pyrazole-3-carboxamide |
| 292 | 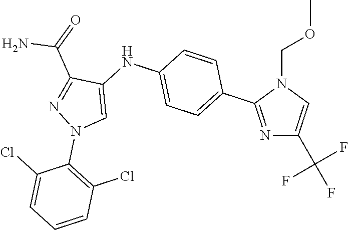 [SMILES: COCn1cc(C(F)(F)F)nc1-c1ccc(Nc2cn(-c3c(Cl)cccc3Cl)nc2C(N)=O)cc1] | 1-(2,6-dichlorophenyl)-4-((4-(1- (methoxymethyl)-4-(trifluoromethyl)- 1H-imidazol-2-yl)phenyl)amino)-1H- pyrazole-3-carboxamide |
| 293 | 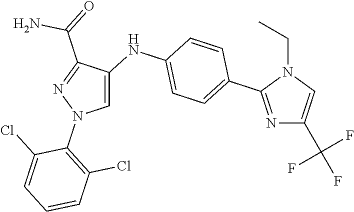 [SMILES: CCn1cc(C(F)(F)F)nc1-c1ccc(Nc2cn(-c3c(Cl)cccc3Cl)nc2C(N)=O)cc1] | 1-(2,6-dichlorophenyl)-4-((4-(1-ethyl- 4-(trifluoromethyl)-1H-imidazol-2- yl)phenyl)amino)-1H-pyrazole-3- carboxamide |
| 294 | 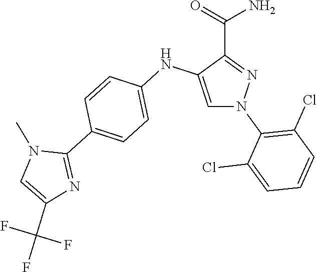 [SMILES: Cn1cc(C(F)(F)F)nc1-c1ccc(Nc2cn(-c3c(Cl)cccc3Cl)nc2C(N)=O)cc1] | 1-(2,6-dichlorophenyl)-4-((4-(1- methyl-4-(trifluoromethyl)-1H- imidazol-2-yl)phenyl)amino)-1H- pyrazole-3-carboxamide |
| 295 | 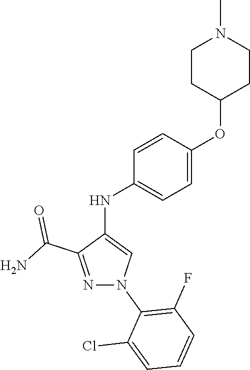 [SMILES: CN1CCC(Oc2ccc(Nc3cn(-c4c(F)cccc4Cl)nc3C(N)=O)cc2)CC1] | 1-(2-chloro-6-fluorophenyl)-4-((4-((1- methylpiperidin-4- yl)oxy)phenyl)amino)-1H-pyrazole-3- carboxamide |
| 296 | 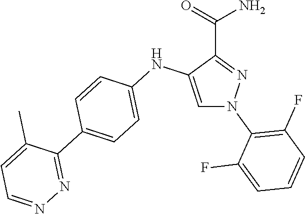 [SMILES: Cc1ccnnc1-c1ccc(Nc2cn(-c3c(F)cccc3F)nc2C(N)=O)cc1] | 1-(2,6-difluorophenyl)-4-((4-(4- methylpyridazin-3-yl)phenyl)amino)- 1H-pyrazole-3-carboxamide |
| 297 | 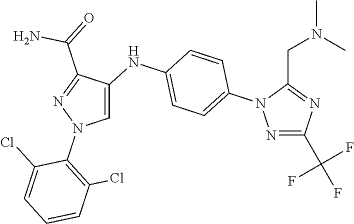 [SMILES: CN(C)Cc1nc(C(F)(F)F)nn1-c1ccc(Nc2cn(-c3c(Cl)cccc3Cl)nc2C(N)=O)cc1] | 1-(2,6-dichlorophenyl)-4-((4-(5- ((dimethylamino)methyl)-3- (trifluoromethyl)-1H-1,2,4-triazol-1- yl)phenyl)amino)-1H-pyrazole-3- carboxamide |
| 298 | 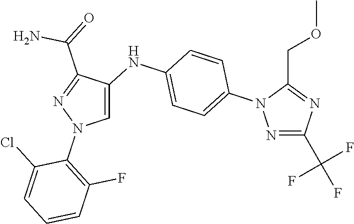 [SMILES: COCc1nc(C(F)(F)F)nn1-c1ccc(Nc2cn(-c3c(F)cccc3Cl)nc2C(N)=O)cc1] | 1-(2-chloro-6-fluorophenyl)-4-((4-(5- (methoxymethyl)-3-(trifluoromethyl)- 1H-1,2,4-triazol-1-yl)phenyl)amino)- 1H-pyrazole-3-carboxamide |
| 299 | 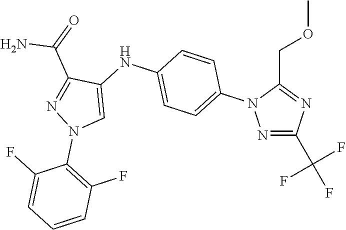 [SMILES: COCc1nc(C(F)(F)F)nn1-c1ccc(Nc2cn(-c3c(F)cccc3F)nc2C(N)=O)cc1] | 1-(2,6-difluorophenyl)-4-((4-(5- (methoxymethyl)-3-(trifluoromethyl)- 1H-1,2,4-triazol-1-yl)phenyl)amino)- 1H-pyrazole-3-carboxamide |
| 300 | 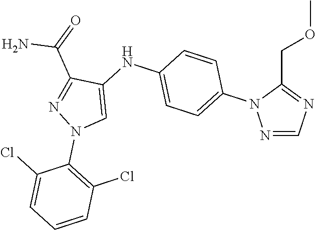 [SMILES: COCc1ncnn1-c1ccc(Nc2cn(-c3c(Cl)cccc3Cl)nc2C(N)=O)cc1] | 1-(2,6-dichlorophenyl)-4-((4-(5- (methoxymethyl)-1H-1,2,4-triazol-1- yl)phenyl)amino)-1H-pyrazole-3- carboxamide |
| 301 | 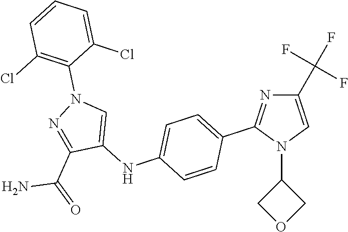 [SMILES: NC(=O)c1nn(-c2c(Cl)cccc2Cl)cc1Nc1ccc(-c2nc(C(F)(F)F)cn2C2COC2)cc1] | 1-(2,6-dichlorophenyl)-4-((4-(1- (oxetan-3-yl)-4-(trifluoromethyl)-1H- imidazol-2-yl)phenyl)amino)-1H- pyrazole-3-carboxamide |
| 302 | 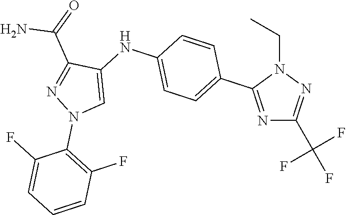 [SMILES: CCn1nc(C(F)(F)F)nc1-c1ccc(Nc2cn(-c3c(F)cccc3F)nc2C(N)=O)cc1] | 1-(2,6-difluorophenyl)-4-((4-(1-ethyl- 3-(trifluoromethyl)-1H-1,2,4-triazol- 5-yl)phenyl)amino)-1H-pyrazole-3- carboxamide |
| 303 | 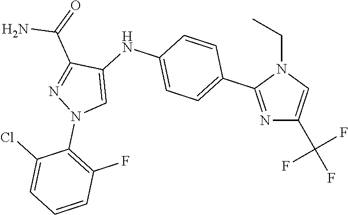 [SMILES: CCn1cc(C(F)(F)F)nc1-c1ccc(Nc2cn(-c3c(F)cccc3Cl)nc2C(N)=O)cc1] | 1-(2-chloro-6-fluorophenyl)-4-((4-(1- ethyl-4-(trifluoromethyl)-1H- imidazol-2-yl)phenyl)amino)-1H- pyrazole-3-carboxamide |
| 304 | 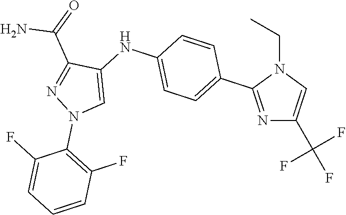 [SMILES: CCn1cc(C(F)(F)F)nc1-c1ccc(Nc2cn(-c3c(F)cccc3F)nc2C(N)=O)cc1] | 1-(2,6-difluorophenyl)-4-((4-(1-ethyl- 4-(trifluoromethyl)-1H-imidazol-2- yl)phenyl)amino)-1H-pyrazole-3- carboxamide |
| 305 | 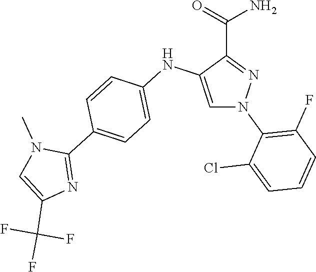 [SMILES: Cn1cc(C(F)(F)F)nc1-c1ccc(Nc2cn(-c3c(F)cccc3Cl)nc2C(N)=O)cc1] | 1-(2-chloro-6-fluorophenyl)-4-((4-(1- methyl-4-(trifluoromethyl)-1H- imidazol-2-yl)phenyl)amino)-1H- pyrazole-3-carboxamide |
| 306 | 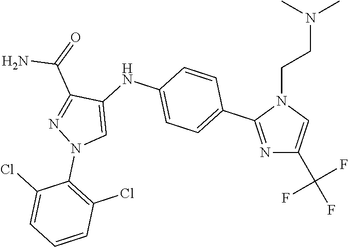 [SMILES: CN(C)CCn1cc(C(F)(F)F)nc1-c1ccc(Nc2cn(-c3c(Cl)cccc3Cl)nc2C(N)=O)cc1] | 1-(2,6-dichlorophenyl)-4-((4-(1-(2- (dimethylamino)ethyl)-4- (trifluoromethyl)-1H-imidazol-2- yl)phenyl)amino)-1H-pyrazole-3- carboxamide |
| 307 | 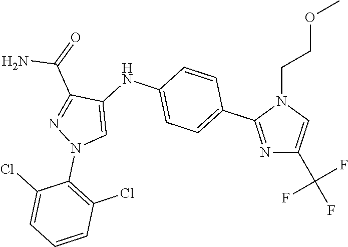 [SMILES: COCCn1cc(C(F)(F)F)nc1-c1ccc(Nc2cn(-c3c(Cl)cccc3Cl)nc2C(N)=O)cc1] | 1-(2,6-dichlorophenyl)-4-((4-(1-(2- methoxyethyl)-4-(trifluoromethyl)- 1H-imidazol-2-yl)phenyl)amino)-1H- pyrazole-3-carboxamide |
| 308 | 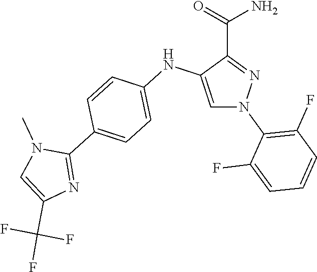 [SMILES: Cn1cc(C(F)(F)F)nc1-c1ccc(Nc2cn(-c3c(F)cccc3F)nc2C(N)=O)cc1] | 1-(2,6-difluorophenyl)-4-((4-(1- methyl-4-(trifluoromethyl)-1H- imidazol-2-yl)phenyl)amino)-1H- pyrazole-3-carboxamide |
| 309 | 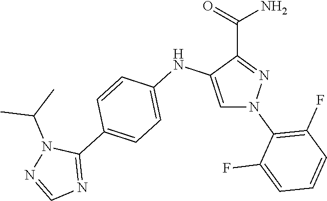 [SMILES: CC(C)n1ncnc1-c1ccc(Nc2cn(-c3c(F)cccc3F)nc2C(N)=O)cc1] | 1-(2,6-difluorophenyl)-4-((4-(1- isopropyl-1H-1,2,4-triazol-5- yl)phenyl)amino)-1H-pyrazole-3- carboxamide |
| 310 | 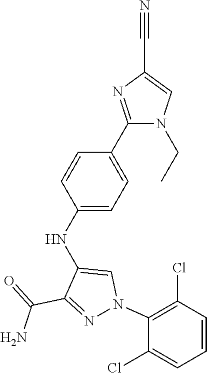 [SMILES: CCn1cc(C#N)nc1-c1ccc(Nc2cn(-c3c(Cl)cccc3Cl)nc2C(N)=O)cc1] | 4-((4-(4-cyano-1-ethyl-1H-imidazol- 2-yl)phenyl)amino)-1-(2,6- dichlorophenyl)-1H-pyrazole-3- carboxamide |
| 311 | 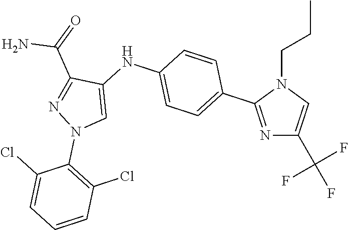 [SMILES: CCCn1cc(C(F)(F)F)nc1-c1ccc(Nc2cn(-c3c(Cl)cccc3Cl)nc2C(N)=O)cc1] | 1-(2,6-dichlorophenyl)-4-((4-(1- propyl-4-(trifluoromethyl)-1H- imidazol-2-yl)phenyl)amino)-1H- pyrazole-3-carboxamide |
| 312 | 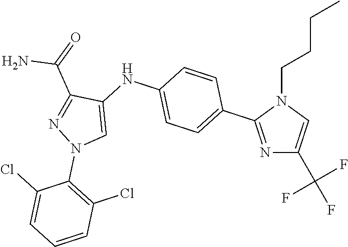 [SMILES: CCCCn1cc(C(F)(F)F)nc1-c1ccc(Nc2cn(-c3c(Cl)cccc3Cl)nc2C(N)=O)cc1] | 4-((4-(1-butyl-4-(trifluoromethyl)-1H- imidazol-2-yl)phenyl)amino)-1-(2,6- dichlorophenyl)-1H-pyrazole-3- carboxamide |
| 313 | 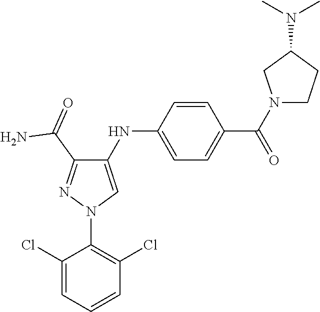 [SMILES: CN(C)[C@@H]1CCN(C(=O)c2ccc(Nc3cn(-c4c(Cl)cccc4Cl)nc3C(N)=O)cc2)C1] | (R)-1-(2,6-dichlorophenyl)-4-((4-(3- (dimethylamino)pyrrolidine-1- carbonyl)phenyl)amino)-1H-pyrazole- 3-carboxamide |
| 314 | 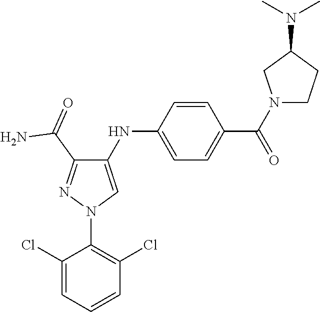 [SMILES: CN(C)[C@H]1CCN(C(=O)c2ccc(Nc3cn(-c4c(Cl)cccc4Cl)nc3C(N)=O)cc2)C1] | (S)-1-(2,6-dichlorophenyl)-4-((4-(3- (dimethylamino)pyrrolidine-1- carbonyl)phenyl)amino)-1H-pyrazole- 3-carboxamide |
| 315 | 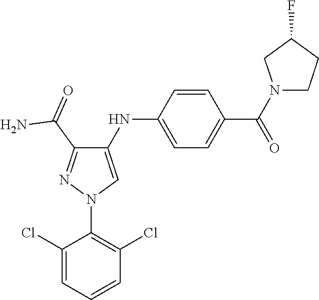 [SMILES: NC(=O)c1nn(-c2c(Cl)cccc2Cl)cc1Nc1ccc(C(=O)N2CC[C@@H](F)C2)cc1] | (R)-1-(2,6-dichlorophenyl)-4-((4-(3- fluoropyrrolidine-1- carbonyl)phenyl)amino)-1H-pyrazole- 3-carboxamide |
| 316 | 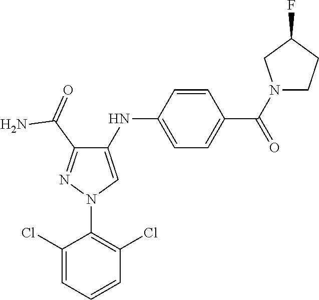 [SMILES: NC(=O)c1nn(-c2c(Cl)cccc2Cl)cc1Nc1ccc(C(=O)N2CC[C@H](F)C2)cc1] | (S)-1-(2,6-dichlorophenyl)-4-((4-(3- fluoropyrrolidine-1- carbonyl)phenyl)amino)-1H-pyrazole- 3-carboxamide |
| 317 | 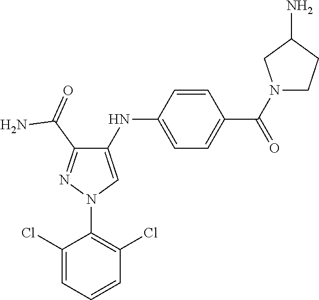 [SMILES: NC(=O)c1nn(-c2c(Cl)cccc2Cl)cc1Nc1ccc(C(=O)N2CCC(N)C2)cc1] | 4-((4-(3-aminopyrrolidine-1- carbonyl)phenyl)amino)-1-(2,6- dichlorophenyl)-1H-pyrazole-3- carboxamide |
| 318 | 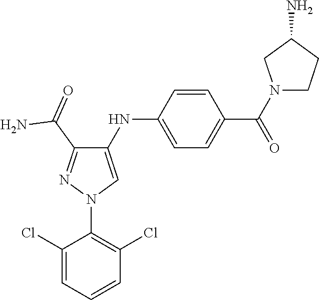 [SMILES: NC(=O)c1nn(-c2c(Cl)cccc2Cl)cc1Nc1ccc(C(=O)N2CC[C@@H](N)C2)cc1] | (R)-4-((4-(3-aminopyrrolidine-1- carbonyl)phenyl)amino)-1-(2,6- dichlorophenyl)-1H-pyrazole-3- carboxamide |
| 319 | 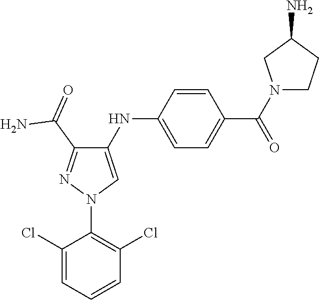 [SMILES: NC(=O)c1nn(-c2c(Cl)cccc2Cl)cc1Nc1ccc(C(=O)N2CC[C@H](N)C2)cc1] | (S)-4-((4-(3-aminopyrrolidine-1- carbonyl)phenyl)amino)-1-(2,6- dichlorophenyl)-1H-pyrazole-3- carboxamide |
| 320 | 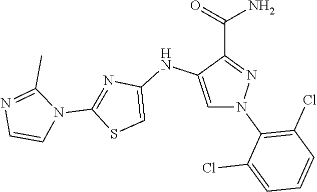 [SMILES: Cc1nccn1-c1nc(Nc2cn(-c3c(Cl)cccc3Cl)nc2C(N)=O)cs1] | 1-(2,6-dichlorophenyl)-4-((2-(2- methyl-1H-imidazol-1-yl)thiazol-4- yl)amino)-1H-pyrazole-3- carboxamide |
| 321 | 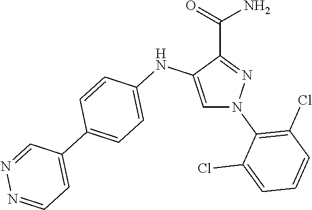 [SMILES: NC(=O)c1nn(-c2c(Cl)cccc2Cl)cc1Nc1ccc(-c2ccnnc2)cc1] | 1-(2,6-dichlorophenyl)-4-((4- (pyridazin-4-yl)phenyl)amino)-1H- pyrazole-3-carboxamide |
| 322 | 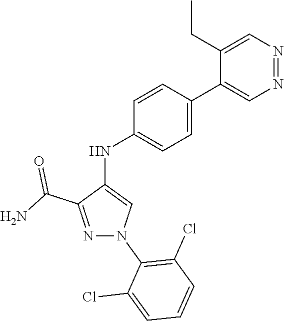 [SMILES: CCc1cnncc1-c1ccc(Nc2cn(-c3c(Cl)cccc3Cl)nc2C(N)=O)cc1] | 1-(2,6-dichlorophenyl)-4-((4-(5- ethylpyridazin-4-yl)phenyl)amino)- 1H-pyrazole-3-carboxamide |
| 323 | 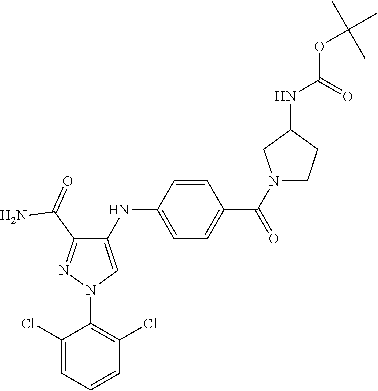 [SMILES: CC(C)(C)OC(=O)NC1CCN(C(=O)c2ccc(Nc3cn(-c4c(Cl)cccc4Cl)nc3C(N)=O)cc2)C1] | tert-butyl_(1-(4-((3-carbamoyl-1-(2,6- dichlorophenyl)-1H-pyrazol-4- yl)amino)benzoyl)pyrrolidin-3- yl)carbamate |
| 324 | 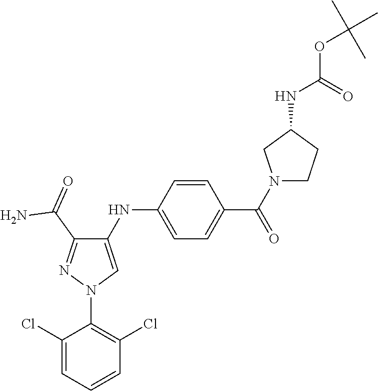 [SMILES: CC(C)(C)OC(=O)N[C@@H]1CCN(C(=O)c2ccc(Nc3cn(-c4c(Cl)cccc4Cl)nc3C(N)=O)cc2)C1] | (R)-tert-butyl_(1-(4-((3-carbamoyl-1- (2,6-dichlorophenyl)-1H-pyrazol-4- yl)amino)benzoyl)pyrrolidin-3- yl)carbamate |
| 325 | 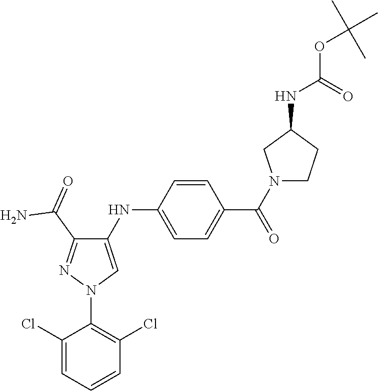 [SMILES: CC(C)(C)OC(=O)N[C@H]1CCN(C(=O)c2ccc(Nc3cn(-c4c(Cl)cccc4Cl)nc3C(N)=O)cc2)C1] | (S)-tert-butyl_(1-(4-((3-carbamoyl-1- (2,6-dichlorophenyl)-1H-pyrazol-4- yl)amino)benzoyl)pyrrolidin-3- yl)carbamate |
| 326 | 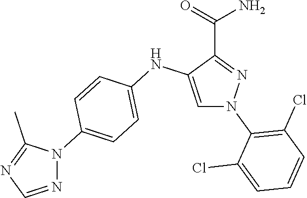 [SMILES: Cc1ncnn1-c1ccc(Nc2cn(-c3c(Cl)cccc3Cl)nc2C(N)=O)cc1] | 1-(2,6-dichlorophenyl)-4-((4-(5- methyl-1H-1,2,4-triazol-1- yl)phenyl)amino)-1H-pyrazole-3- carboxamide |
| 327 | 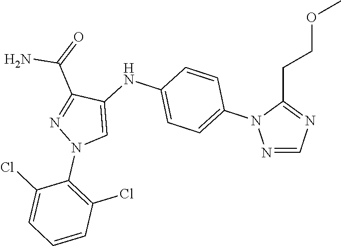 [SMILES: COCCc1ncnn1-c1ccc(Nc2cn(-c3c(Cl)cccc3Cl)nc2C(N)=O)cc1] | 1-(2,6-dichlorophenyl)-4-((4-(5-(2- methoxyethyl)-1H-1,2,4-triazol-1- yl)phenyl)amino)-1H-pyrazole-3- carboxamide |
| 328 | 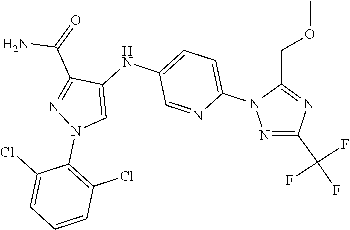 [SMILES: COCc1nc(C(F)(F)F)nn1-c1ccc(Nc2cn(-c3c(Cl)cccc3Cl)nc2C(N)=O)cn1] | 1-(2,6-dichlorophenyl)-4-((6-(5- (methoxymethyl)-3-(trifluoromethyl)- 1H-1,2,4-triazol-1-yl)pyridin-3- yl)amino)-1H-pyrazole-3- carboxamide |
| 329 | 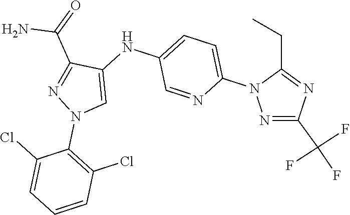 [SMILES: CCc1nc(C(F)(F)F)nn1-c1ccc(Nc2cn(-c3c(Cl)cccc3Cl)nc2C(N)=O)cn1] | 1-(2,6-dichlorophenyl)-4-((6-(5-ethyl- 3-(trifluoromethyl)-1H-1,2,4-triazol- 1-yl)pyridin-3-yl)amino)-1H- pyrazole-3-carboxamide |
| 330 | 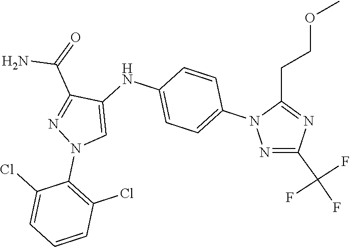 [SMILES: COCCc1nc(C(F)(F)F)nn1-c1ccc(Nc2cn(-c3c(Cl)cccc3Cl)nc2C(N)=O)cc1] | 1-(2,6-dichlorophenyl)-4-((4-(5-(2- methoxyethyl)-3-(trifluoromethyl)- 1H-1,2,4-triazol-1-yl)phenyl)amino)- 1H-pyrazole-3-carboxamide |
| 331 | 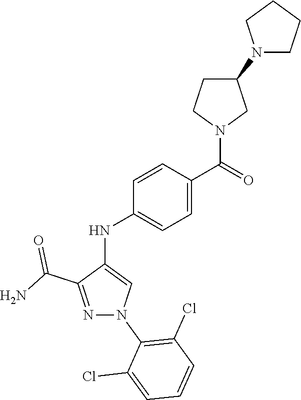 [SMILES: NC(=O)c1nn(-c2c(Cl)cccc2Cl)cc1Nc1ccc(C(=O)N2CC[C@@H](N3CCCC3)C2)cc1] | (R)-4-((4-([1,3′-bipyrrolidine]-1′- carbonyl)phenyl)amino)-1-(2,6- dichlorophenyl)-1H-pyrazole-3- carboxamide |
| 332 | 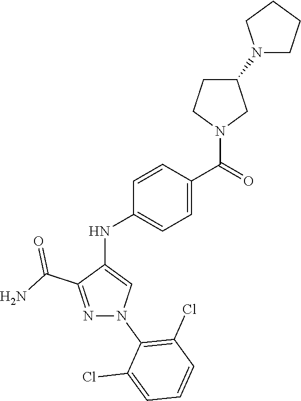 [SMILES: NC(=O)c1nn(-c2c(Cl)cccc2Cl)cc1Nc1ccc(C(=O)N2CC[C@H](N3CCCC3)C2)cc1] | (S)-4-((4-([1,3′-bipyrrolidine]-1′- carbonyl)phenyl)amino)-1-(2,6- dichlorophenyl)-1H-pyrazole-3- carboxamide |
| 333 | 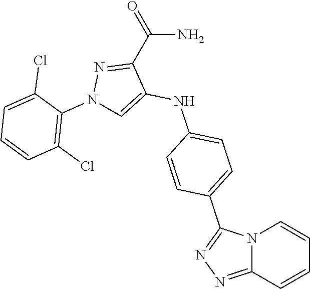 [SMILES: NC(=O)c1nn(-c2c(Cl)cccc2Cl)cc1Nc1ccc(-c2nnc3ccccn23)cc1] | 4-((4-([1,2,4]triazolo[4,3-a]pyridin-3- yl)phenyl)amino)-1-(2,6- dichlorophenyl)-1H-pyrazole-3- carboxamide |
| 334 | 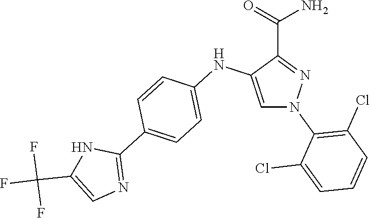 [SMILES: NC(=O)c1nn(-c2c(Cl)cccc2Cl)cc1Nc1ccc(-c2ncc(C(F)(F)F)[nH]2)cc1] | 1-(2,6-dichlorophenyl)-4-((4-(5- (trifluoromethyl)-1H-1,2,4-triazol-3- yl)phenyl)amino)-1H-pyrazole-3- carboxamide |
| 335 | 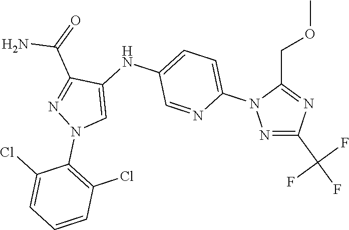 [SMILES: COCc1nc(C(F)(F)F)nn1-c1ccc(Nc2cn(-c3c(Cl)cccc3Cl)nc2C(N)=O)cn1] | 1-(2,6-dichlorophenyl)-4-((6-(5- (methoxymethyl)-3-(trifluoromethyl)- 1H-1,2,4-triazol-1-yl)pyridazin-3- yl)amino)-1H-pyrazole-3- carboxamide |
| 336 | 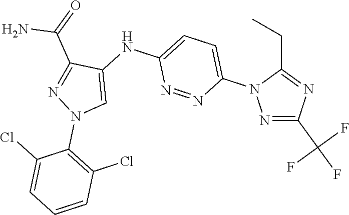 [SMILES: CCc1nc(C(F)(F)F)nn1-c1ccc(Nc2cn(-c3c(Cl)cccc3Cl)nc2C(N)=O)nn1] | 1-(2,6-dichlorophenyl)-4-((6-(5-ethyl- 3-(trifluoromethyl)-1H-1,2,4-triazol- 1-yl)pyridin-3-yl)amino)-1H- pyrazole-3-carboxamide |
| 337 | 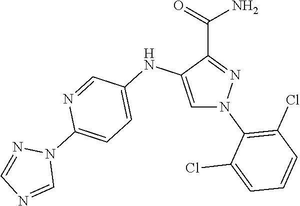 [SMILES: NC(=O)c1nn(-c2c(Cl)cccc2Cl)cc1Nc1ccc(-n2cncn2)nc1] | 4-((6-(1H-1,2,4-triazol-1-yl)pyridin-3- yl)amino)-1-(2,6-dichlorophenyl)-1H- pyrazole-3-carboxamide |
| 338 | 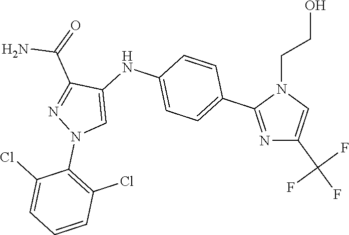 [SMILES: NC(=O)c1nn(-c2c(Cl)cccc2Cl)cc1Nc1ccc(-c2nc(C(F)(F)F)cn2CCO)cc1] | 1-(2,6-dichlorophenyl)-4-((4-(1-(2- hydroxyethyl)-4-(trifluoromethyl)-1H- imidazol-2-yl)phenyl)amino)-1H- pyrazole-3-carboxamide |
| 339 | 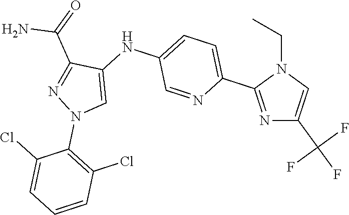 [SMILES: CCn1cc(C(F)(F)F)nc1-c1ccc(Nc2cn(-c3c(Cl)cccc3Cl)nc2C(N)=O)cn1] | 1-(2,6-dichlorophenyl)-4-((6-(1-ethyl- 4-(trifluoromethyl)-1H-imidazol-2- yl)pyridin-3-yl)amino)-1H-pyrazole- 3-carboxamide |
| 340 | 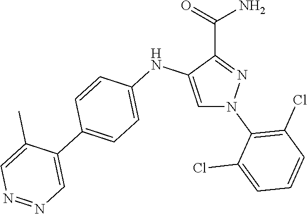 [SMILES: Cc1cnncc1-c1ccc(Nc2cn(-c3c(Cl)cccc3Cl)nc2C(N)=O)cc1] | 1-(2,6-dichlorophenyl)-4-((4-(5- methylpyridazin-4-yl)phenyl)amino)- 1H-pyrazole-3-carboxamide |
| 341 | 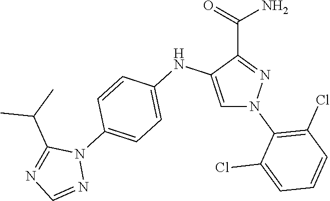 [SMILES: CC(C)c1ncnn1-c1ccc(Nc2cn(-c3c(Cl)cccc3Cl)nc2C(N)=O)cc1] | 1-(2,6-dichlorophenyl)-4-((4-(5- isopropyl-1H-1,2,4-triazol-1- yl)phenyl)amino)-1H-pyrazole-3- carboxamide |
| 342 | 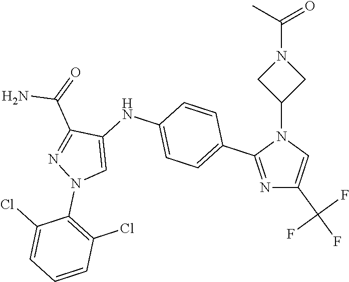 [SMILES: CC(=O)N1CC(n2cc(C(F)(F)F)nc2-c2ccc(Nc3cn(-c4c(Cl)cccc4Cl)nc3C(N)=O)cc2)C1] | 4-((4-(1-(1-acetylazetidin-3-yl)-4- (trifluoromethyl)-1H-imidazol-2- yl)phenyl)amino)-1-(2,6- dichlorophenyl)-1H-pyrazole-3- carboxamide |
| 343 | 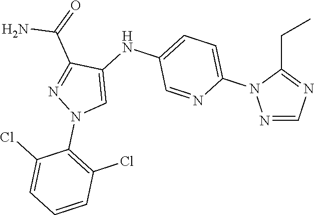 [SMILES: CCc1ncnn1-c1ccc(Nc2cn(-c3c(Cl)cccc3Cl)nc2C(N)=O)cn1] | 1-(2,6-dichlorophenyl)-4-((6-(5-ethyl- 1H-1,2,4-triazol-1-yl)pyridin-3- yl)amino)-1H-pyrazole-3- carboxamide |
| 344 | 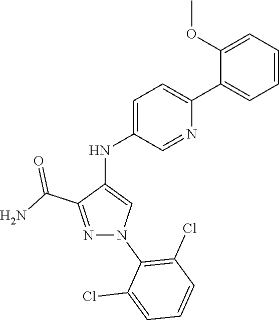 [SMILES: COc1ccccc1-c1ccc(Nc2cn(-c3c(Cl)cccc3Cl)nc2C(N)=O)cn1] | 1-(2,6-dichlorophenyl)-4-((6-(2- methoxyphenyl)pyridin-3-yl)amino)- 1H-pyrazole-3-carboxamide |
| 345 | 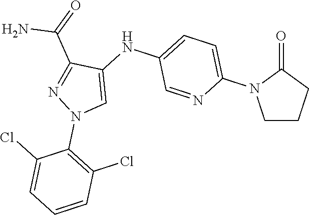 [SMILES: NC(=O)c1nn(-c2c(Cl)cccc2Cl)cc1Nc1ccc(N2CCCC2=O)nc1] | 1-(2,6-dichlorophenyl)-4-((6-(2- oxopyrrolidin-1-yl)pyridin-3- yl)amino)-1H-pyrazole-3- carboxamide |
| 346 | 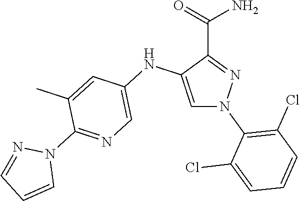 [SMILES: Cc1cc(Nc2cn(-c3c(Cl)cccc3Cl)nc2C(N)=O)cnc1-n1cccn1] | 1-(2,6-dichlorophenyl)-4-((5-methyl- 6-(1H-pyrazol-1-yl)pyridin-3- yl)amino)-1H-pyrazole-3- carboxamide |
| 347 | 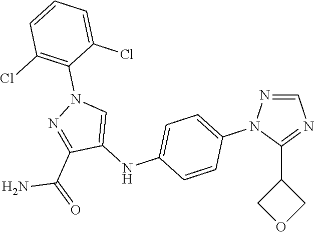 [SMILES: NC(=O)c1nn(-c2c(Cl)cccc2Cl)cc1Nc1ccc(-n2ncnc2C2COC2)cc1] | 1-(2,6-dichlorophenyl)-4-((4-(5- (oxetan-3-yl)-1H-1,2,4-triazol-1- yl)phenyl)amino)-1H-pyrazole-3- carboxamide |
| 348 | 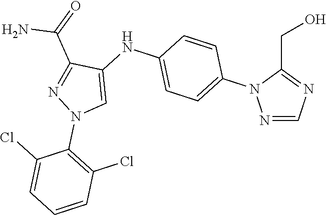 [SMILES: NC(=O)c1nn(-c2c(Cl)cccc2Cl)cc1Nc1ccc(-n2ncnc2CO)cc1] | 1-(2,6-dichlorophenyl)-4-((4-(5- (hydroxymethyl)-1H-1,2,4-triazol-1- yl)phenyl)amino)-1H-pyrazole-3- carboxamide |
| 349 | 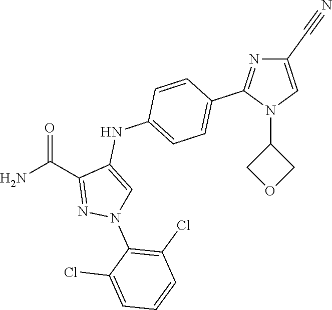 [SMILES: N#Cc1cn(C2COC2)c(-c2ccc(Nc3cn(-c4c(Cl)cccc4Cl)nc3C(N)=O)cc2)n1] | 4-((4-(4-cyano-1-(oxetan-3-yl)-1H- imidazol-2-yl)phenyl)amino)-1-(2,6- dichlorophenyl)-1H-pyrazole-3- carboxamide |
| 350 | 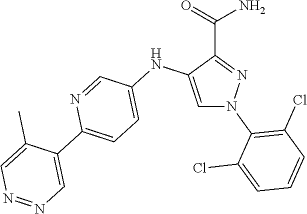 [SMILES: Cc1cnncc1-c1ccc(Nc2cn(-c3c(Cl)cccc3Cl)nc2C(N)=O)cn1] | 1-(2,6-dichlorophenyl)-4-((6-(5- methylpyridazin-4-yl)pyridin-3- yl)amino)-1H-pyrazole-3- carboxamide |
| 351 | 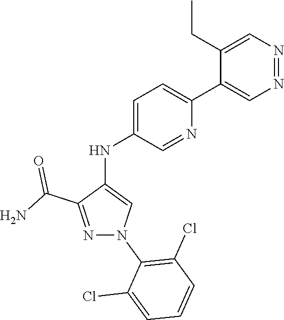 [SMILES: CCc1cnncc1-c1ccc(Nc2cn(-c3c(Cl)cccc3Cl)nc2C(N)=O)cn1] | 1-(2,6-dichlorophenyl)-4-((6-(5- ethylpyridazin-4-yl)pyridin-3- yl)amino)-1H-pyrazole-3- carboxamide |
| 352 | 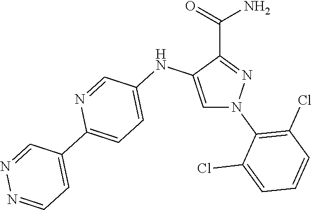 [SMILES: NC(=O)c1nn(-c2c(Cl)cccc2Cl)cc1Nc1ccc(-c2ccnnc2)nc1] | 1-(2,6-dichlorophenyl)-4-((6- (pyridazin-4-yl)pyridin-3-yl)amino)- 1H-pyrazole-3-carboxamide |
| 353 | 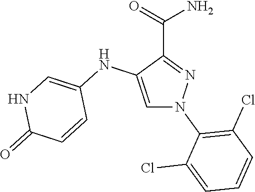 [SMILES: NC(=O)c1nn(-c2c(Cl)cccc2Cl)cc1Nc1ccc(=O)[nH]c1] | 1-(2,6-dichlorophenyl)-4-((6-oxo-1,6- dihydropyridin-3-yl)amino)-1H- pyrazole-3-carboxamide |
| 354 | 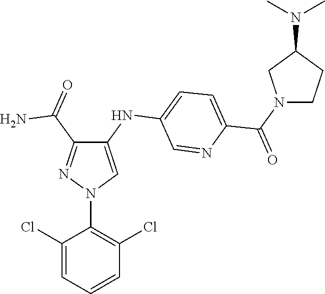 [SMILES: CN(C)[C@H]1CCN(C(=O)c2ccc(Nc3cn(-c4c(Cl)cccc4Cl)nc3C(N)=O)cn2)C1] | (S)-1-(2,6-dichlorophenyl)-4-((6-(3- (dimethylamino)pyrrolidine-1- carbonyl)pyridin-3-yl)amino)-1H- pyrazole-3-carboxamide |
| 355 | 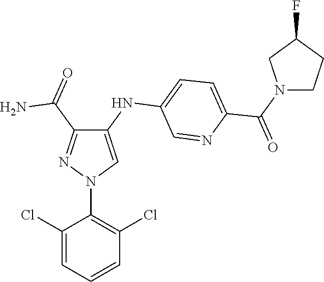 [SMILES: NC(=O)c1nn(-c2c(Cl)cccc2Cl)cc1Nc1ccc(C(=O)N2CC[C@H](F)C2)nc1] | (S)-1-(2,6-dichlorophenyl)-4-((6-(3- fluoropyrrolidine-1-carbonyl)pyridin- 3-yl)amino)-1H-pyrazole-3- carboxamide |
| 356 | 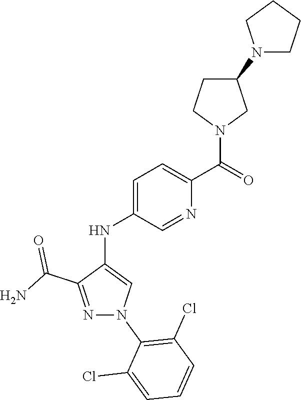 [SMILES: NC(=O)c1nn(-c2c(Cl)cccc2Cl)cc1Nc1ccc(C(=O)N2CC[C@@H](N3CCCC3)C2)nc1] | (R)-4-((6-([1,3′-bipyrrolidine]-1′- carbonyl)pyridin-3-yl)amino)-1-(2,6- dichlorophenyl)-1H-pyrazole-3- carboxamide |
| 357 | 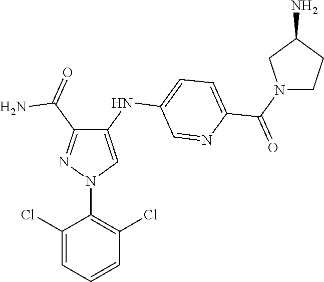 [SMILES: NC(=O)c1nn(-c2c(Cl)cccc2Cl)cc1Nc1ccc(C(=O)N2CC[C@H](N)C2)nc1] | (S)-4-((6-(3-aminopyrrolidine-1- carbonyl)pyridin-3-yl)amino)-1-(2,6- dichlorophenyl)-1H-pyrazole-3- carboxamide |
| 358 | 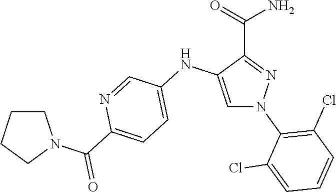 [SMILES: NC(=O)c1nn(-c2c(Cl)cccc2Cl)cc1Nc1ccc(C(=O)N2CCCC2)nc1] | 1-(2,6-dichlorophenyl)-4-((6- (pyrrolidine-1-carbonyl)pyridin-3- yl)amino)-1H-pyrazole-3- carboxamide |
| 359 | 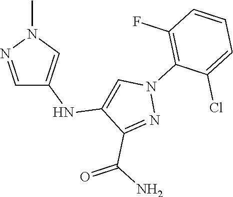 [SMILES: Cn1cc(Nc2cn(-c3c(F)cccc3Cl)nc2C(N)=O)cn1] | 1-(2-chloro-6-fluorophenyl)-4-((1- methyl-1H-pyrazol-4-yl)amino)-1H- pyrazole-3-carboxamide |
| 360 | 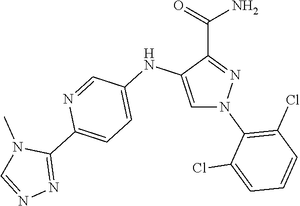 [SMILES: Cn1cnnc1-c1ccc(Nc2cn(-c3c(Cl)cccc3Cl)nc2C(N)=O)cn1] | 1-(2,6-dichlorophenyl)-4-((6-(4- methyl-4H-1,2,4-triazol-3-yl)pyridin- 3-yl)amino)-1H-pyrazole-3- carboxamide |
| 361 | 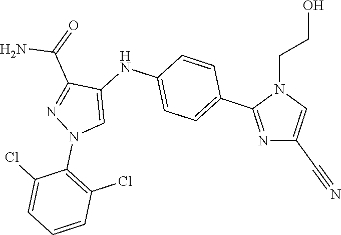 [SMILES: N#Cc1cn(CCO)c(-c2ccc(Nc3cn(-c4c(Cl)cccc4Cl)nc3C(N)=O)cc2)n1] | 4-((4-(4-cyano-1-(2-hydroxyethyl)- 1H-imidazol-2-yl)phenyl)amino)-1- (2,6-dichlorophenyl)-1H-pyrazole-3- carboxamide |
| 362 | 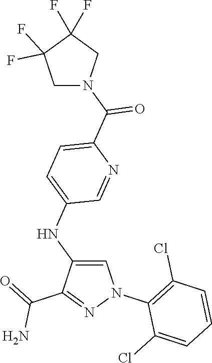 [SMILES: NC(=O)c1nn(-c2c(Cl)cccc2Cl)cc1Nc1ccc(C(=O)N2CC(F)(F)C(F)(F)C2)nc1] | 1-(2,6-dichlorophenyl)-4-((6-(3,3,4,4- tetrafluoropyrrolidine-1- carbonyl)pyridin-3-yl)amino)-1H- pyrazole-3-carboxamide |
| 363 | 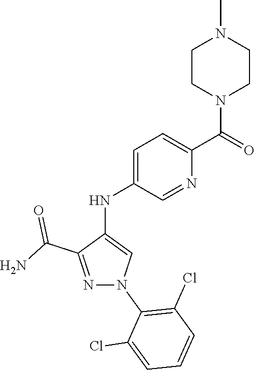 [SMILES: CN1CCN(C(=O)c2ccc(Nc3cn(-c4c(Cl)cccc4Cl)nc3C(N)=O)cn2)CC1] | 1-(2,6-dichlorophenyl)-4-((6-(4- methylpiperazine-1-carbonyl)pyridin- 3-yl)amino)-1H-pyrazole-3- carboxamide |
| 364 | 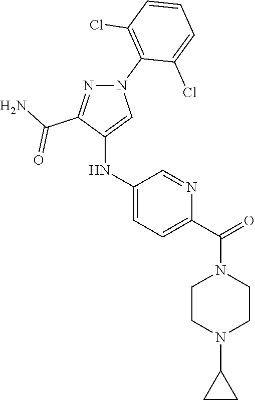 [SMILES: NC(=O)c1nn(-c2c(Cl)cccc2Cl)cc1Nc1ccc(C(=O)N2CCN(C3CC3)CC2)nc1] | 4-((6-(4-cyclopropylpiperazine-1- carbonyl)pyridin-3-yl)amino)-1-(2,6- dichlorophenyl)-1H-pyrazole-3- carboxamide |
| 365 | 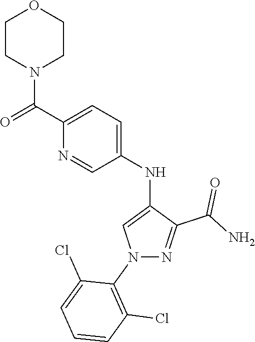 [SMILES: NC(=O)c1nn(-c2c(Cl)cccc2Cl)cc1Nc1ccc(C(=O)N2CCOCC2)nc1] | 1-(2,6-dichlorophenyl)-4-((6- (morpholine-4-carbonyl)pyridin-3- yl)amino)-1H-pyrazole-3- carboxamide |
| 366 | 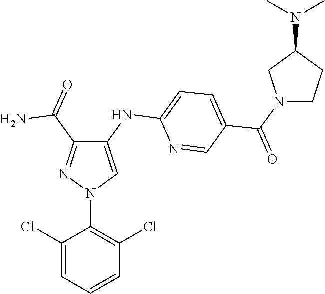 [SMILES: CN(C)[C@H]1CCN(C(=O)c2ccc(Nc3cn(-c4c(Cl)cccc4Cl)nc3C(N)=O)nc2)C1] | (S)-1-(2,6-dichlorophenyl)-4-((5-(3- (dimethylamino)pyrrolidine-1- carbonyl)pyridin-2-yl)amino)-1H- pyrazole-3-carboxamide |
| 367 | 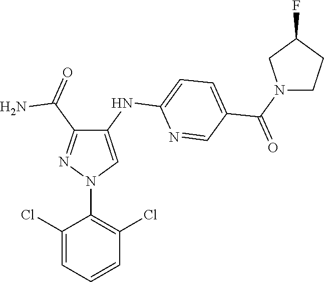 [SMILES: NC(=O)c1nn(-c2c(Cl)cccc2Cl)cc1Nc1ccc(C(=O)N2CC[C@H](F)C2)cn1] | (S)-1-(2,6-dichlorophenyl)-4-((5-(3- fluoropyrrolidine-1-carbonyl)pyridin- 2-yl)amino)-1H-pyrazole-3- carboxamide |
| 368 | 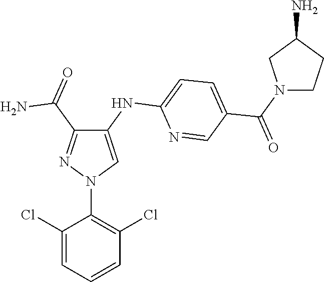 [SMILES: NC(=O)c1nn(-c2c(Cl)cccc2Cl)cc1Nc1ccc(C(=O)N2CC[C@H](N)C2)cn1] | (S)-4-((5-(3-aminopyrrolidine-1- carbonyl)pyridin-2-yl)amino)-1-(2,6- dichlorophenyl)-1H-pyrazole-3- carboxamide |
| 369 | 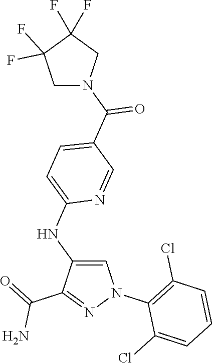 [SMILES: NC(=O)c1nn(-c2c(Cl)cccc2Cl)cc1Nc1ccc(C(=O)N2CC(F)(F)C(F)(F)C2)cn1] | 1-(2,6-dichlorophenyl)-4-((5-(3,3,4,4- tetrafluoropyrrolidine-1- carbonyl)pyridin-2-yl)amino)-1H- pyrazole-3-carboxamide |
| 370 | 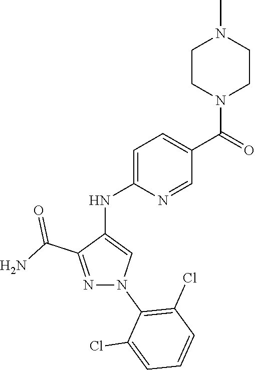 [SMILES: CN1CCN(C(=O)c2ccc(Nc3cn(-c4c(Cl)cccc4Cl)nc3C(N)=O)nc2)CC1] | 1-(2,6-dichlorophenyl)-4-((5-(4- methylpiperazine-1-carbonyl)pyridin- 2-yl)amino)-1H-pyrazole-3- carboxamide |
| 371 | 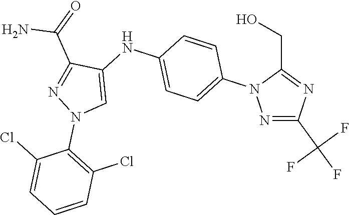 [SMILES: NC(=O)c1nn(-c2c(Cl)cccc2Cl)cc1Nc1ccc(-n2nc(C(F)(F)F)nc2CO)cc1] | 1-(2,6-dichlorophenyl)-4-((4-(5- (hydroxymethyl)-3-(trifluoromethyl)- 1H-1,2,4-triazol-1-yl)phenyl)amino)- 1H-pyrazole-3-carboxamide |
| 372 | 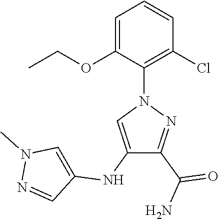 [SMILES: CCOc1cccc(Cl)c1-n1cc(Nc2cnn(C)c2)c(C(N)=O)n1] | 1-(2-chloro-6-ethoxyphenyl)-4-((1- methyl-1H-pyrazol-4-yl)amino)-1H- pyrazole-3-carboxamide |
| 373 | 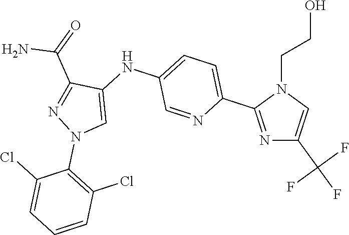 [SMILES: NC(=O)c1nn(-c2c(Cl)cccc2Cl)cc1Nc1ccc(-c2nc(C(F)(F)F)cn2CCO)nc1] | 1-(2,6-dichlorophenyl)-4-((6-(1-(2- hydroxyethyl)-4-(trifluoromethyl)-1H- imidazol-2-yl)pyridin-3-yl)amino)- 1H-pyrazole-3-carboxamide |
| 374 | 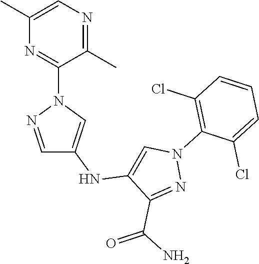 [SMILES: Cc1cnc(C)c(-n2cc(Nc3cn(-c4c(Cl)cccc4Cl)nc3C(N)=O)cn2)n1] | 1-(2,6-dichlorophenyl)-4-((1-(3,6- dimethylpyrazin-2-yl)-1H-pyrazol-4- yl)amino)-1H-pyrazole-3- carboxamide |
| 375 | 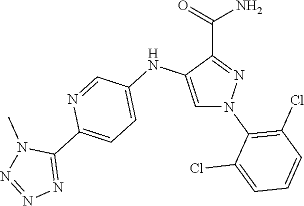 [SMILES: Cn1nnnc1-c1ccc(Nc2cn(-c3c(Cl)cccc3Cl)nc2C(N)=O)cn1] | 1-(2,6-dichlorophenyl)-4-((6-(1- methyl-1H-tetrazol-5-yl)pyridin-3- yl)amino)-1H-pyrazole-3- carboxamide |
| 376 | 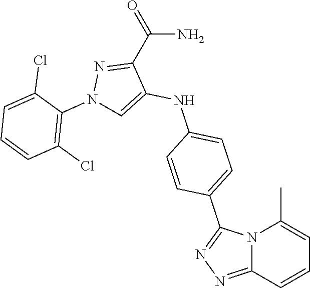 [SMILES: Cc1cccc2nnc(-c3ccc(Nc4cn(-c5c(Cl)cccc5Cl)nc4C(N)=O)cc3)n12] | 1-(2,6-dichlorophenyl)-4-((4-(5- methyl-[1,2,4]triazolo[4,3-a]pyridin- 3-yl)phenyl)amino)-1H-pyrazole-3- carboxamide |
| 377 | 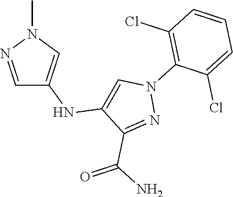 [SMILES: Cn1cc(Nc2cn(-c3c(Cl)cccc3Cl)nc2C(N)=O)cn1] | 1-(2,6-dichlorophenyl)-4-((1-methyl- 1H-pyrazol-4-yl)amino)-1H-pyrazole- 3-carboxamide |
| 378 | 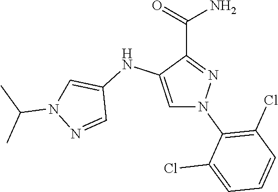 [SMILES: CC(C)n1cc(Nc2cn(-c3c(Cl)cccc3Cl)nc2C(N)=O)cn1] | 1-(2,6-dichlorophenyl)-4-((1- isopropyl-1H-pyrazol-4-yl)amino)- 1H-pyrazole-3-carboxamide |
| 379 | 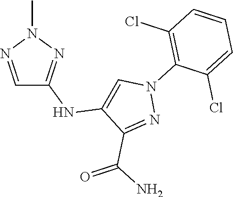 [SMILES: Cn1ncc(Nc2cn(-c3c(Cl)cccc3Cl)nc2C(N)=O)n1] | 1-(2,6-dichlorophenyl)-4-((2-methyl- 2H-1,2,3-triazol-4-yl)amino)-1H- pyrazole-3-carboxamide |
| 380 | 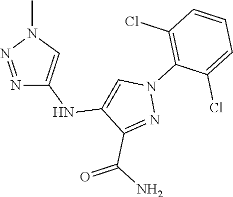 [SMILES: Cn1cc(Nc2cn(-c3c(Cl)cccc3Cl)nc2C(N)=O)nn1] | 1-(2,6-dichlorophenyl)-4-((1-methyl- 1H-1,2,3-triazol-4-yl)amino)-1H- pyrazole-3-carboxamide |
| 381 | 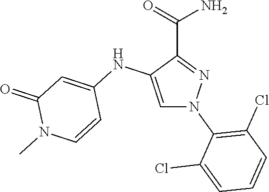 [SMILES: Cn1ccc(Nc2cn(-c3c(Cl)cccc3Cl)nc2C(N)=O)cc1=O] | 1-(2,6-dichlorophenyl)-4-((1-methyl- 2-oxo-1,2-dihydropyridin-4- yl)amino)-1H-pyrazole-3- carboxamide |
| 382 | 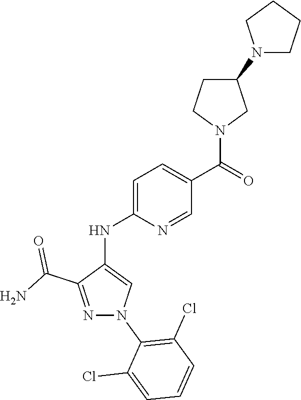 [SMILES: NC(=O)c1nn(-c2c(Cl)cccc2Cl)cc1Nc1ccc(C(=O)N2CC[C@@H](N3CCCC3)C2)cn1] | (R)-4-((5-([1,3′-bipyrrolidine]-1′- carbonyl)pyridin-2-yl)amino)-1-(2,6- dichlorophenyl)-1H-pyrazole-3- carboxamide |
| 383 | 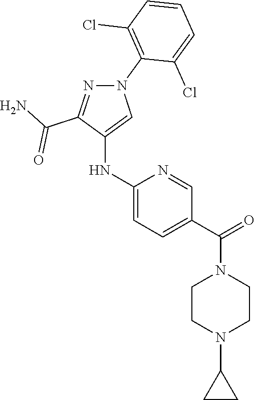 [SMILES: NC(=O)c1nn(-c2c(Cl)cccc2Cl)cc1Nc1ccc(C(=O)N2CCN(C3CC3)CC2)cn1] | 4-((5-(4-cyclopropylpiperazine-1- carbonyl)pyridin-2-yl)amino)-1-(2,6- dichlorophenyl)-1H-pyrazole-3- carboxamide |
| 384 | 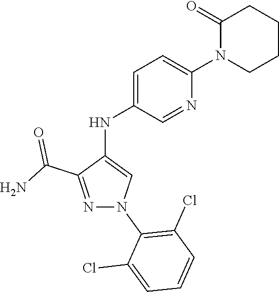 [SMILES: NC(=O)c1nn(-c2c(Cl)cccc2Cl)cc1Nc1ccc(N2CCCCC2=O)nc1] | 1-(2,6-dichlorophenyl)-4-((6-(2- oxopiperidin-1-yl)pyridin-3- yl)amino)-1H-pyrazole-3- carboxamide |
| 385 | 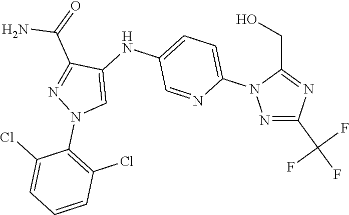 [SMILES: NC(=O)c1nn(-c2c(Cl)cccc2Cl)cc1Nc1ccc(-n2nc(C(F)(F)F)nc2CO)nc1] | 1-(2,6-dichlorophenyl)-4-((6-(5- (hydroxymethyl)-3-(trifluoromethyl)- 1H-1,2,4-triazol-1-yl)pyridin-3- yl)amino)-1H-pyrazole-3- carboxamide |
| 386 | 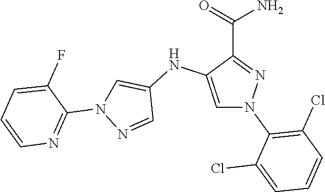 [SMILES: NC(=O)c1nn(-c2c(Cl)cccc2Cl)cc1Nc1cnn(-c2ncccc2F)c1] | 1-(2,6-dichlorophenyl)-4-((1-(3- fluoropyridin-2-yl)-1H-pyrazol-4- yl)amino)-1H-pyrazole-3- carboxamide |
| 387 | 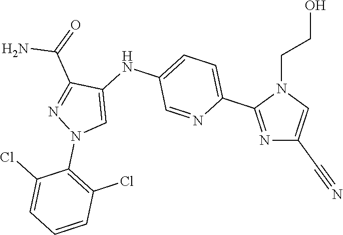 [SMILES: N#Cc1cn(CCO)c(-c2ccc(Nc3cn(-c4c(Cl)cccc4Cl)nc3C(N)=O)cn2)n1] | 4-((6-(4-cyano-1-(2-hydroxyethyl)- 1H-imidazol-2-yl)pyridin-3- yl)amino)-1-(2,6-dichlorophenyl)-1H- pyrazole-3-carboxamide |
| 388 | 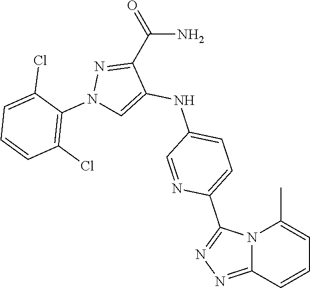 [SMILES: Cc1cccc2nnc(-c3ccc(Nc4cn(-c5c(Cl)cccc5Cl)nc4C(N)=O)cn3)n12] | 1-(2,6-dichlorophenyl)-4-((6-(5- methyl-[1,2,4]triazolo[4,3-a]pyridin- 3-yl)pyridin-3-yl)amino)-1H- pyrazole-3-carboxamide |
| 389 | 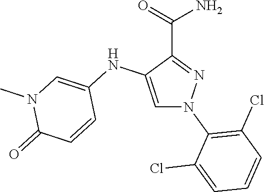 [SMILES: Cn1cc(Nc2cn(-c3c(Cl)cccc3Cl)nc2C(N)=O)ccc1=O] | 1-(2,6-dichlorophenyl)-4-((1-methyl- 6-oxo-1,6-dihydropyridin-3- yl)amino)-1H-pyrazole-3- carboxamide |
| 390 | 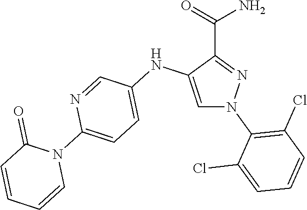 [SMILES: NC(=O)c1nn(-c2c(Cl)cccc2Cl)cc1Nc1ccc(-n2ccccc2=O)nc1] | 1-(2,6-dichlorophenyl)-4-((2-oxo-2H- [1,2′-bipyridin]-5′-yl)amino)-1H- pyrazole-3-carboxamide |
| 391 | 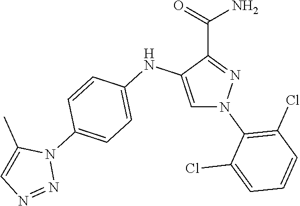 [SMILES: Cc1cnnn1-c1ccc(Nc2cn(-c3c(Cl)cccc3Cl)nc2C(N)=O)cc1] | 1-(2,6-dichlorophenyl)-4-((4-(5- methyl-1H-1,2,3-triazol-1- yl)phenyl)amino)-1H-pyrazole-3- carboxamide |
| 392 | 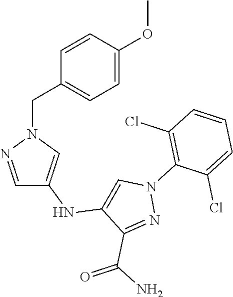 [SMILES: COc1ccc(Cn2cc(Nc3cn(-c4c(Cl)cccc4Cl)nc3C(N)=O)cn2)cc1] | 1-(2,6-dichlorophenyl)-4-((1-(4- methoxybenzyl)-1H-pyrazol-4- yl)amino)-1H-pyrazole-3- carboxamide |
| 393 | 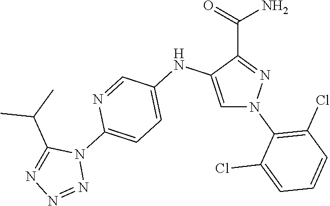 [SMILES: CC(C)c1nnnn1-c1ccc(Nc2cn(-c3c(Cl)cccc3Cl)nc2C(N)=O)cn1] | 1-(2,6-dichlorophenyl)-4-((6-(5- isopropyl-1H-tetrazol-1-yl)pyridin-3- yl)amino)-1H-pyrazole-3- carboxamide |
| 394 | 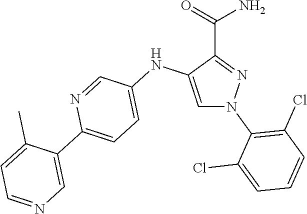 [SMILES: Cc1ccncc1-c1ccc(Nc2cn(-c3c(Cl)cccc3Cl)nc2C(N)=O)cn1] | 1-(2,6-dichlorophenyl)-4-((4′-methyl- [2,3′-bipyridin]-5-yl)amino)-1H- pyrazole-3-carboxamide |
| 395 | 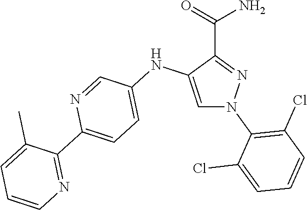 [SMILES: Cc1cccnc1-c1ccc(Nc2cn(-c3c(Cl)cccc3Cl)nc2C(N)=O)cn1] | 1-(2,6-dichlorophenyl)-4-((3′-methyl- [2,2′-bipyridin]-5-yl)amino)-1H- pyrazole-3-carboxamide |
| 396 | 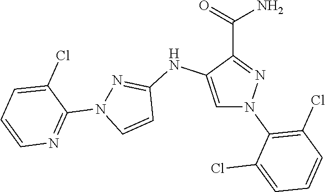 [SMILES: NC(=O)c1nn(-c2c(Cl)cccc2Cl)cc1Nc1ccn(-c2ncccc2Cl)n1] | 4-((1-(3-chloropyridin-2-yl)-1H- pyrazol-3-yl)amino)-1-(2,6- dichlorophenyl)-1H-pyrazole-3- carboxamide |
| 397 | 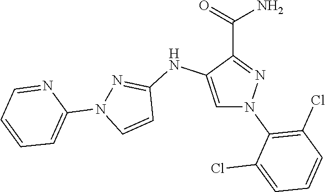 [SMILES: NC(=O)c1nn(-c2c(Cl)cccc2Cl)cc1Nc1ccn(-c2ccccn2)n1] | 1-(2,6-dichlorophenyl)-4-((1-(pyridin- 2-yl)-1H-pyrazol-3-yl)amino)-1H- pyrazole-3-carboxamide |
| 398 | 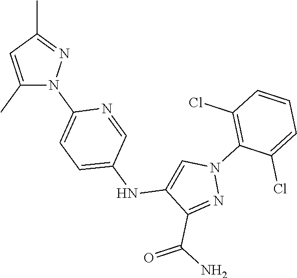 [SMILES: Cc1cc(C)n(-c2ccc(Nc3cn(-c4c(Cl)cccc4Cl)nc3C(N)=O)cn2)n1] | 1-(2,6-dichlorophenyl)-4-((6-(3,5- dimethyl-1H-pyrazol-1-yl)pyridin-3- yl)amino)-1H-pyrazole-3- carboxamide |
| 399 | 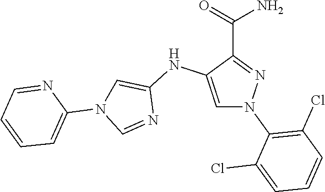 [SMILES: NC(=O)c1nn(-c2c(Cl)cccc2Cl)cc1Nc1cn(-c2ccccn2)cn1] | 1-(2,6-dichlorophenyl)-4-((1-(pyridin- 2-yl)-1H-imidazol-4-yl)amino)-1H- pyrazole-3-carboxamide |
| 400 | 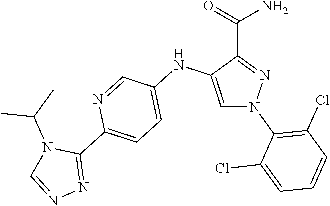 [SMILES: CC(C)n1cnnc1-c1ccc(Nc2cn(-c3c(Cl)cccc3Cl)nc2C(N)=O)cn1] | 1-(2,6-dichlorophenyl)-4-((6-(4- isopropyl-4H-1,2,4-triazol-3- yl)pyridin-3-yl)amino)-1H-pyrazole- 3-carboxamide |
| 401 | 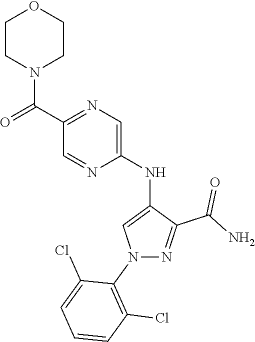 [SMILES: NC(=O)c1nn(-c2c(Cl)cccc2Cl)cc1Nc1cnc(C(=O)N2CCOCC2)cn1] | 1-(2,6-dichlorophenyl)-4-((5- (morpholine-4-carbonyl)pyrazin-2- yl)amino)-1H-pyrazole-3- carboxamide |
| 402 | 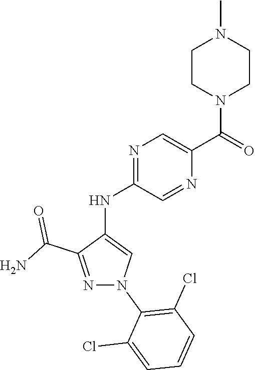 [SMILES: CN1CCN(C(=O)c2cnc(Nc3cn(-c4c(Cl)cccc4Cl)nc3C(N)=O)cn2)CC1] | 1-(2,6-dichlorophenyl)-4-((5-(4- methylpiperazine-1-carbonyl)pyrazin- 2-yl)amino)-1H-pyrazole-3- carboxamide |
| 403 | 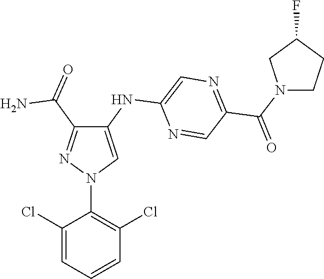 [SMILES: NC(=O)c1nn(-c2c(Cl)cccc2Cl)cc1Nc1cnc(C(=O)N2CC[C@@H](F)C2)cn1] | (R)-1-(2,6-dichlorophenyl)-4-((5-(3- fluoropyrrolidine-1-carbonyl)pyrazin- 2-yl)amino)-1H-pyrazole-3- carboxamide |
| 404 | 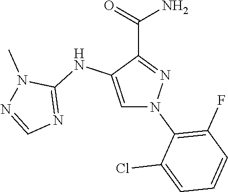 [SMILES: Cn1ncnc1Nc1cn(-c2c(F)cccc2Cl)nc1C(N)=O] | 1-(2-chloro-6-fluorophenyl)-4-((1- methyl-1H-1,2,4-triazol-5-yl)amino)- 1H-pyrazole-3-carboxamide |
| 405 | 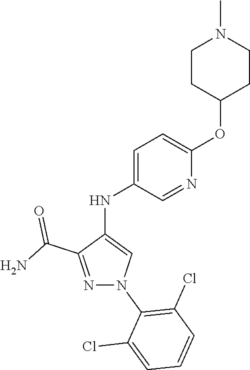 [SMILES: CN1CCC(Oc2ccc(Nc3cn(-c4c(Cl)cccc4Cl)nc3C(N)=O)cn2)CC1] | 1-(2,6-dichlorophenyl)-4-((6-((1- methylpiperidin-4-yl)oxy)pyridin-3- yl)amino)-1H-pyrazole-3- carboxamide |
| 406 | 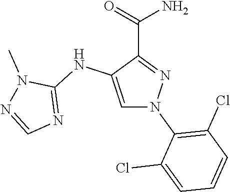 [SMILES: Cn1ncnc1Nc1cn(-c2c(Cl)cccc2Cl)nc1C(N)=O] | 1-(2,6-dichlorophenyl)-4-((1-methyl- 1H-1,2,4-triazol-5-yl)amino)-1H- pyrazole-3-carboxamide |
| 407 | 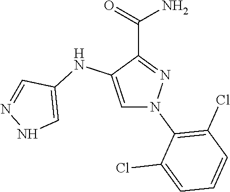 [SMILES: NC(=O)c1nn(-c2c(Cl)cccc2Cl)cc1Nc1cn[nH]c1] | 4-((1H-pyrazol-4-yl)amino)-1-(2,6- dichlorophenyl)-1H-pyrazole-3- carboxamide |
| 408 | 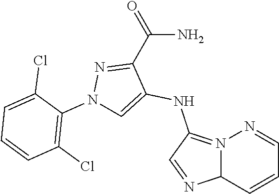 | 1-(2,6-dichlorophenyl)-4- (imidazo[1,2-b]pyridazin-3-ylamino)- 1H-pyrazole-3-carboxamide |
| 409 | 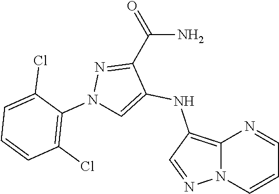 [SMILES: NC(=O)c1nn(-c2c(Cl)cccc2Cl)cc1Nc1cnn2cccnc12] | 1-(2,6-dichlorophenyl)-4- (pyrazolo[1,5-a]pyrimidin-3- ylamino)-1H-pyrazole-3-carboxamide |
| 410 | 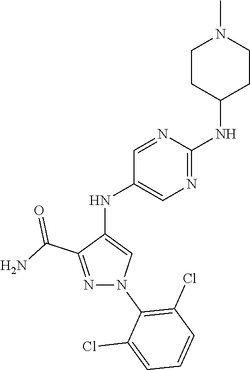 [SMILES: CN1CCC(Nc2ncc(Nc3cn(-c4c(Cl)cccc4Cl)nc3C(N)=O)cn2)CC1] | 1-(2,6-dichlorophenyl)-4-((2-((1- methylpiperidin-4- yl)amino)pyrimidin-5-yl)amino)-1H- pyrazole-3-carboxamide |
| 411 | 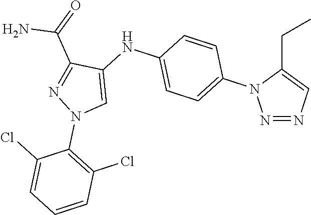 [SMILES: CCc1cnnn1-c1ccc(Nc2cn(-c3c(Cl)cccc3Cl)nc2C(N)=O)cc1] | 1-(2,6-dichlorophenyl)-4-((4-(5-ethyl- 1H-1,2,3-triazol-1-yl)phenyl)amino)- 1H-pyrazole-3-carboxamide |
| 412 | 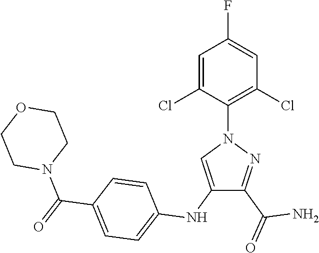 [SMILES: NC(=O)c1nn(-c2c(Cl)cc(F)cc2Cl)cc1Nc1ccc(C(=O)N2CCOCC2)cc1] | 1-(2,6-dichloro-4-fluorophenyl)-4-((4- (morpholine-4- carbonyl)phenyl)amino)-1H-pyrazole- 3-carboxamide |
| 413 | 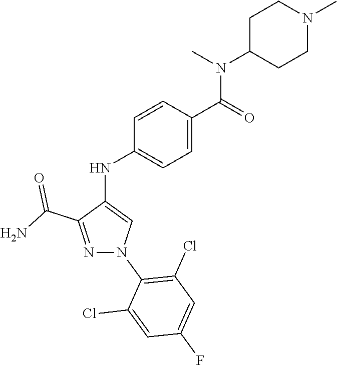 [SMILES: CN1CCC(N(C)C(=O)c2ccc(Nc3cn(-c4c(Cl)cc(F)cc4Cl)nc3C(N)=O)cc2)CC1] | 1-(2,6-dichloro-4-fluorophenyl)-4-((4- (methyl(1-methylpiperidin-4- yl)carbamoyl)phenyl)amino)-1H- pyrazole-3-carboxamide |
| 414 | 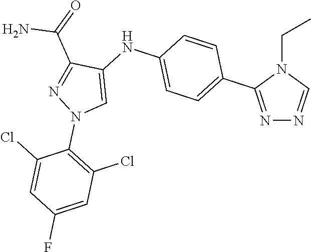 [SMILES: CCn1cnnc1-c1ccc(Nc2cn(-c3c(Cl)cc(F)cc3Cl)nc2C(N)=O)cc1] | 1-(2,6-dichloro-4-fluorophenyl)-4-((4- (4-ethyl-4H-1,2,4-triazol-3- yl)phenyl)amino)-1H-pyrazole-3- carboxamide |
| 415 | 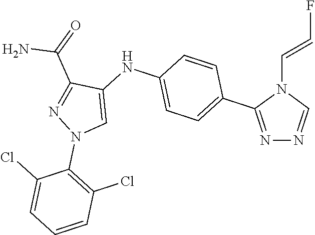 [SMILES: NC(=O)c1nn(-c2c(Cl)cccc2Cl)cc1Nc1ccc(-c2nncn2/C=C/F)cc1] | (E)-1-(2,6-dichlorophenyl)-4-((4-(4- (2-fluorovinyl)-4H-1,2,4-triazol-3- yl)phenyl)amino)-1H-pyrazole-3- carboxamide |
| 416 | 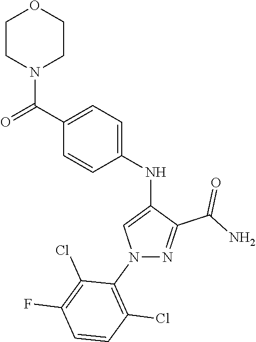 [SMILES: NC(=O)c1nn(-c2c(Cl)ccc(F)c2Cl)cc1Nc1ccc(C(=O)N2CCOCC2)cc1] | 1-(2,6-dichloro-3-fluorophenyl)-4-((4- (morpholine-4- carbonyl)phenyl)amino)-1H-pyrazole- 3-carboxamide |
| 417 | 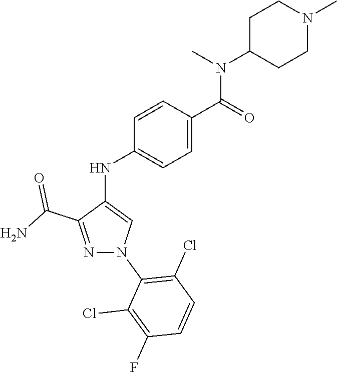 [SMILES: CN1CCC(N(C)C(=O)c2ccc(Nc3cn(-c4c(Cl)ccc(F)c4Cl)nc3C(N)=O)cc2)CC1] | 1-(2,6-dichloro-3-fluorophenyl)-4-((4- (methyl(1-methylpiperidin-4- yl)carbamoyl)phenyl)amino)-1H- pyrazole-3-carboxamide |
| 418 | 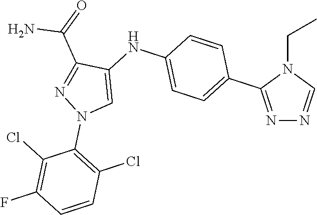 [SMILES: CCn1cnnc1-c1ccc(Nc2cn(-c3c(Cl)ccc(F)c3Cl)nc2C(N)=O)cc1] | 1-(2,6-dichloro-3-fluorophenyl)-4-((4- (4-ethyl-4H-1,2,4-triazol-3- yl)phenyl)amino)-1H-pyrazole-3- carboxamide |
| 419 | 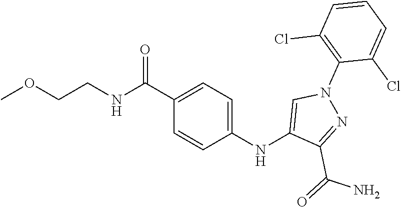 [SMILES: COCCNC(=O)c1ccc(Nc2cn(-c3c(Cl)cccc3Cl)nc2C(N)=O)cc1] | 1-(2,6-dichlorophenyl)-4-((4-((2- methoxyethyl)carbamoyl)phenyl) amino)-1H-pyrazole-3-carboxamide |
| 420 | 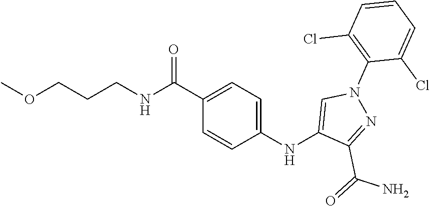 [SMILES: COCCCNC(=O)c1ccc(Nc2cn(-c3c(Cl)cccc3Cl)nc2C(N)=O)cc1] | 1-(2,6-dichlorophenyl)-4-((4-((3- methoxypropyl)carbamoyl)phenyl) amino)-1H-pyrazole-3-carboxamide |
| 421 | 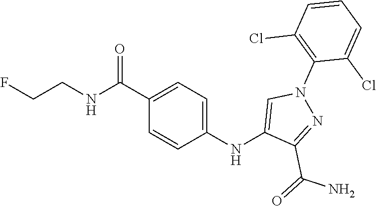 [SMILES: NC(=O)c1nn(-c2c(Cl)cccc2Cl)cc1Nc1ccc(C(=O)NCCF)cc1] | 1-(2,6-dichlorophenyl)-4-((4-((2- fluoroethyl)carbamoyl)phenyl)amino)- 1H-pyrazole-3-carboxamide |
| 422 | 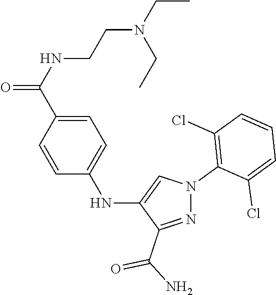 [SMILES: CCN(CC)CCNC(=O)c1ccc(Nc2cn(-c3c(Cl)cccc3Cl)nc2C(N)=O)cc1] | 1-(2,6-dichlorophenyl)-4-((4-((2- (diethylamino)ethyl)carbamoyl) phenyl)amino)-1H-pyrazole-3-carboxamide |
| 423 | 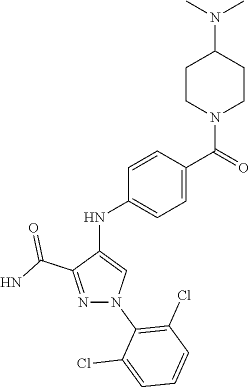 [SMILES: CN(C)C1CCN(C(=O)c2ccc(Nc3cn(-c4c(Cl)cccc4Cl)nc3C(N)=O)cc2)CC1] | 1-(2,6-dichlorophenyl)-4-((4-(4- (dimethylamino)piperidine-1- carbonyl)phenyl)amino)-1H-pyrazole- 3-carboxamide |
| 424 | 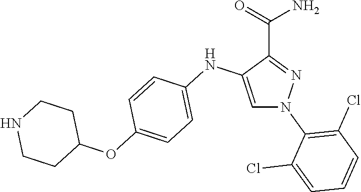 [SMILES: NC(=O)c1nn(-c2c(Cl)cccc2Cl)cc1Nc1ccc(OC2CCNCC2)cc1] | 1-(2,6-dichlorophenyl)-4-((4- (piperidin-4-yloxy)phenyl)amino)-1H- pyrazole-3-carboxamide |
| 425 | 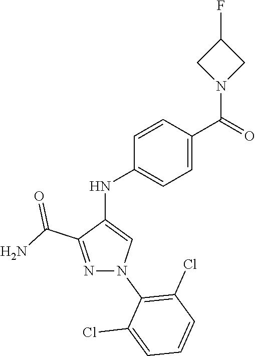 [SMILES: NC(=O)c1nn(-c2c(Cl)cccc2Cl)cc1Nc1ccc(C(=O)N2CC(F)C2)cc1] | 1-(2,6-dichlorophenyl)-4-((4-(3- fluoroazetidine-1- carbonyl)phenyl)amino)-1H-pyrazole- 3-carboxamide |
| 426 | 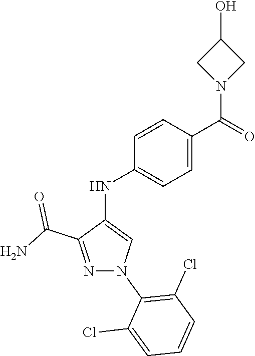 [SMILES: NC(=O)c1nn(-c2c(Cl)cccc2Cl)cc1Nc1ccc(C(=O)N2CC(O)C2)cc1] | 1-(2,6-dichlorophenyl)-4-((4-(3- hydroxyazetidine-1- carbonyl)phenyl)amino)-1H-pyrazole- 3-carboxamide |
| 427 | 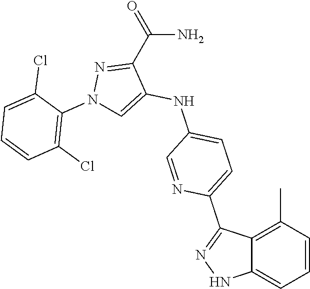 [SMILES: Cc1cccc2[nH]nc(-c3ccc(Nc4cn(-c5c(Cl)cccc5Cl)nc4C(N)=O)cn3)c12] | 1-(2,6-dichlorophenyl)-4-((6-(4- methyl-3H-indazol-3-ylidene)-1,6- dihydropyridin-3-yl)amino)-1H- pyrazole-3-carboxamide |
| 428 | 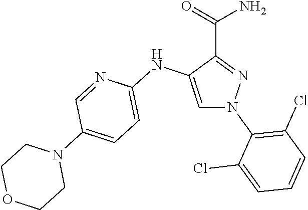 [SMILES: NC(=O)c1nn(-c2c(Cl)cccc2Cl)cc1Nc1ccc(N2CCOCC2)cn1] | 1-(2,6-dichlorophenyl)-4-((5- morpholinopyridin-2-yl)amino)-1H- pyrazole-3-carboxamide |
| 429 | 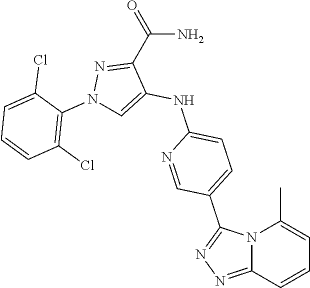 [SMILES: Cc1cccc2nnc(-c3ccc(Nc4cn(-c5c(Cl)cccc5Cl)nc4C(N)=O)nc3)n12] | 1-(2,6-dichlorophenyl)-4-((5-(5- methyl-[1,2,4]triazolo[4,3-a]pyridin- 3-yl)pyridin-2-yl)amino)-1H- pyrazole-3-carboxamide |
| 430 | 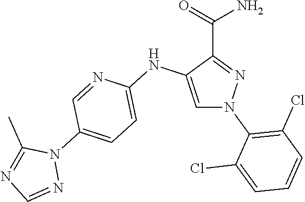 [SMILES: Cc1ncnn1-c1ccc(Nc2cn(-c3c(Cl)cccc3Cl)nc2C(N)=O)nc1] | 1-(2,6-dichlorophenyl)-4-((5-(5- methyl-1H-1,2,4-triazol-1-yl)pyridin- 2-yl)amino)-1H-pyrazole-3- carboxamide |
| 431 | 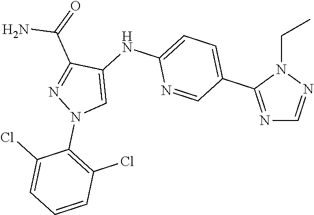 [SMILES: CCn1ncnc1-c1ccc(Nc2cn(-c3c(Cl)cccc3Cl)nc2C(N)=O)nc1] | 1-(2,6-dichlorophenyl)-4-((5-(1-ethyl- 1H-1,2,4-triazol-5-yl)pyridin-2- yl)amino)-1H-pyrazole-3- carboxamide |
| 432 | 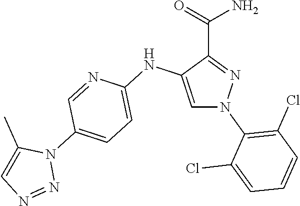 [SMILES: Cc1cnnn1-c1ccc(Nc2cn(-c3c(Cl)cccc3Cl)nc2C(N)=O)nc1] | 1-(2,6-dichlorophenyl)-4-((5-(5- methyl-1H-1,2,3-triazol-1-yl)pyridin- 2-yl)amino)-1H-pyrazole-3- carboxamide |
| 433 | 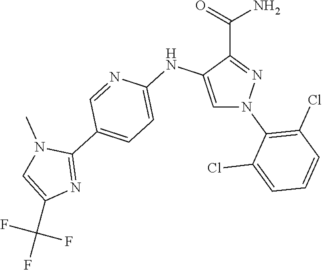 [SMILES: Cn1cc(C(F)(F)F)nc1-c1ccc(Nc2cn(-c3c(Cl)cccc3Cl)nc2C(N)=O)nc1] | 1-(2,6-dichlorophenyl)-4-((5-(1- methyl-4-(trifluoromethyl)-1H- imidazol-2-yl)pyridin-2-yl)amino)- 1H-pyrazole-3-carboxamide |
| 434 | 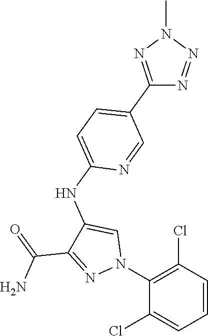 [SMILES: Cn1nnc(-c2ccc(Nc3cn(-c4c(Cl)cccc4Cl)nc3C(N)=O)nc2)n1] | 1-(2,6-dichlorophenyl)-4-((5-(2- methyl-2H-tetrazol-5-yl)pyridin-2- yl)amino)-1H-pyrazole-3- carboxamide |
| 435 | 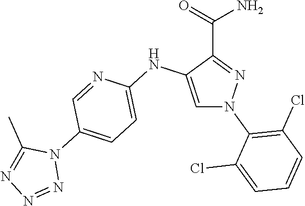 [SMILES: Cc1nnnn1-c1ccc(Nc2cn(-c3c(Cl)cccc3Cl)nc2C(N)=O)nc1] | 1-(2,6-dichlorophenyl)-4-((5-(5- methyl-1H-tetrazol-1-yl)pyridin-2- yl)amino)-1H-pyrazole-3- carboxamide |
| 436 | 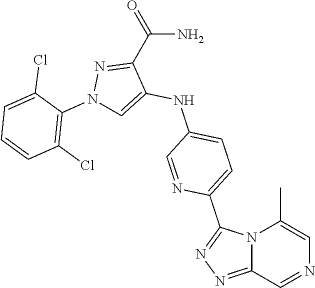 [SMILES: Cc1cncc2nnc(-c3ccc(Nc4cn(-c5c(Cl)cccc5Cl)nc4C(N)=O)cn3)n12] | 1-(2,6-dichlorophenyl)-4-((6-(5- methyl-[1,2,4]triazolo[4,3-a]pyrazin- 3-yl)pyridin-3-yl)amino)-1H- pyrazole-3-carboxamide |
| 437 | 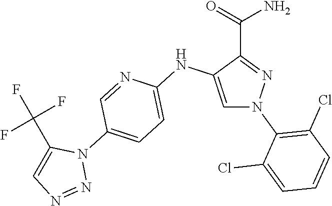 [SMILES: NC(=O)c1nn(-c2c(Cl)cccc2Cl)cc1Nc1ccc(-n2nncc2C(F)(F)F)cn1] | 1-(2,6-dichlorophenyl)-4-((5-(5- (trifluoromethyl)-1H-1,2,3-triazol-1- yl)pyridin-2-yl)amino)-1H-pyrazole- 3-carboxamide |
| 438 | 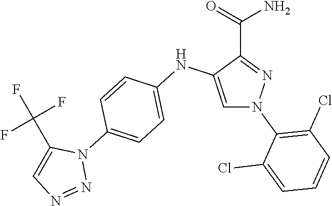 [SMILES: NC(=O)c1nn(-c2c(Cl)cccc2Cl)cc1Nc1ccc(-n2nncc2C(F)(F)F)cc1] | 1-(2,6-dichlorophenyl)-4-((4-(5- (trifluoromethyl)-1H-1,2,3-triazol-1- yl)phenyl)amino)-1H-pyrazole-3- carboxamide |
| 439 | 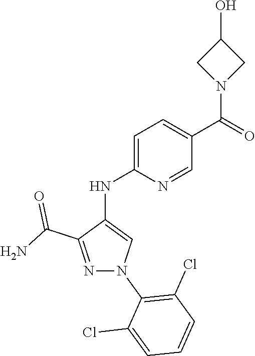 [SMILES: NC(=O)c1nn(-c2c(Cl)cccc2Cl)cc1Nc1ccc(C(=O)N2CC(O)C2)cn1] | 1-(2,6-dichlorophenyl)-4-((5-(3- hydroxyazetidine-1-carbonyl)pyridin- 2-yl)amino)-1H-pyrazole-3- carboxamide |
| 440 | 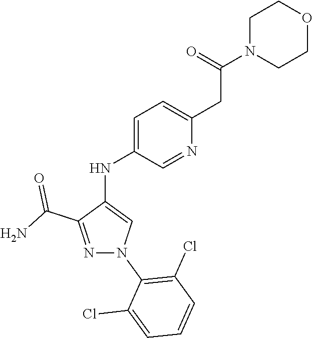 [SMILES: NC(=O)c1nn(-c2c(Cl)cccc2Cl)cc1Nc1ccc(CC(=O)N2CCOCC2)nc1] | 1-(2,6-dichlorophenyl)-4-((6-(2- morpholino-2-oxoethyl)pyridin-3- yl)amino)-1H-pyrazole-3- carboxamide |
| 441 | 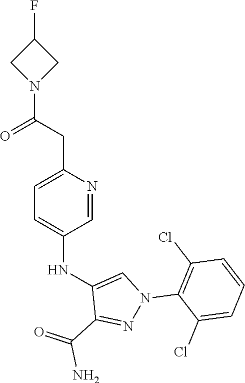 [SMILES: NC(=O)c1nn(-c2c(Cl)cccc2Cl)cc1Nc1ccc(CC(=O)N2CC(F)C2)nc1] | 1-(2,6-dichlorophenyl)-4-((6-(2-(3- fluoroazetidin-1-yl)-2- oxoethyl)pyridin-3-yl)amino)-1H- pyrazole-3-carboxamide |
| 442 | 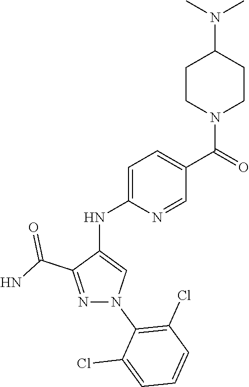 [SMILES: CN(C)C1CCN(C(=O)c2ccc(Nc3cn(-c4c(Cl)cccc4Cl)nc3C(N)=O)nc2)CC1] | 1-(2,6-dichlorophenyl)-4-((5-(4- (dimethylamino)piperidine-1- carbonyl)pyridin-2-yl)amino)-1H- pyrazole-3-carboxamide |
| 443 | 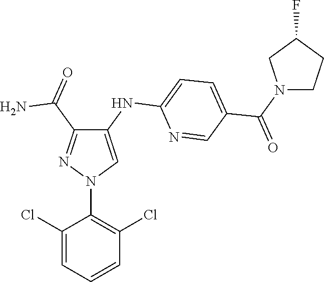 [SMILES: NC(=O)c1nn(-c2c(Cl)cccc2Cl)cc1Nc1ccc(C(=O)N2CC[C@@H](F)C2)cn1] | (R)-1-(2,6-dichlorophenyl)-4-((5-(3- fluoropyrrolidine-1-carbonyl)pyridin- 2-yl)amino)-1H-pyrazole-3- carboxamide |
| 444 | 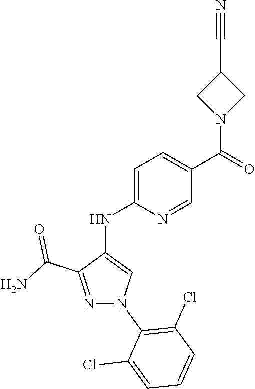 [SMILES: N#CC1CN(C(=O)c2ccc(Nc3cn(-c4c(Cl)cccc4Cl)nc3C(N)=O)nc2)C1] | 4-((5-(3-cyanoazetidine-1- carbonyl)pyridin-2-yl)amino)-1-(2,6- dichlorophenyl)-1H-pyrazole-3- carboxamide |
| 445 | 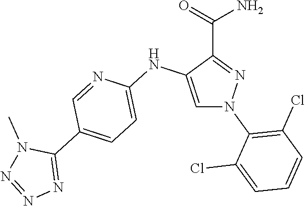 [SMILES: Cn1nnnc1-c1ccc(Nc2cn(-c3c(Cl)cccc3Cl)nc2C(N)=O)nc1] | 1-(2,6-dichlorophenyl)-4-((5-(1- methyl-1H-tetrazol-5-yl)pyridin-2- yl)amino)-1H-pyrazole-3- carboxamide |
| 446 | 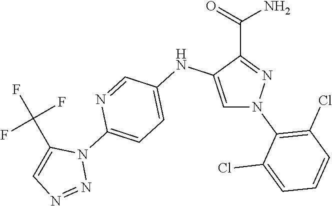 [SMILES: NC(=O)c1nn(-c2c(Cl)cccc2Cl)cc1Nc1ccc(-n2nncc2C(F)(F)F)nc1] | 1-(2,6-dichlorophenyl)-4-((6-(5- (trifluoromethyl)-1H-1,2,3-triazol-1- yl)pyridin-3-yl)amino)-1H-pyrazole- 3-carboxamide |
| 447 | 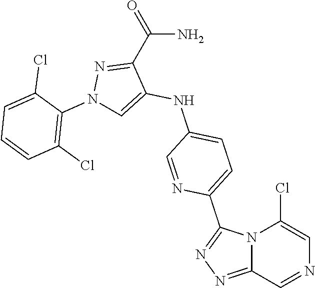 [SMILES: NC(=O)c1nn(-c2c(Cl)cccc2Cl)cc1Nc1ccc(-c2nnc3cncc(Cl)n23)nc1] | 4-((6-(5-chloro-[1,2,4]triazolo[4,3- a]pyrazin-3-yl)pyridin-3-yl)amino)-1- (2,6-dichlorophenyl)-1H-pyrazole-3- carboxamide |
| 448 | 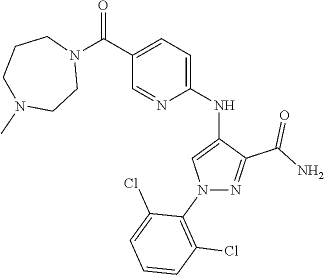 [SMILES: CN1CCCN(C(=O)c2ccc(Nc3cn(-c4c(Cl)cccc4Cl)nc3C(N)=O)nc2)CC1] | 1-(2,6-dichlorophenyl)-4-((5-(4- methyl-1,4-diazepane-1- carbonyl)pyridin-2-yl)amino)-1H- pyrazole-3-carboxamide |
| 449 | 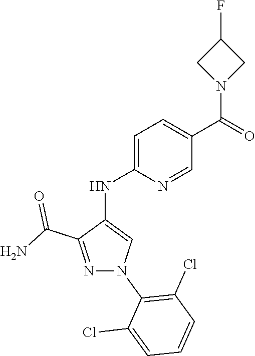 [SMILES: NC(=O)c1nn(-c2c(Cl)cccc2Cl)cc1Nc1ccc(C(=O)N2CC(F)C2)cn1] | 1-(2,6-dichlorophenyl)-4-((5-(3- fluoroazetidine-1-carbonyl)pyridin-2- yl)amino)-1H-pyrazole-3- carboxamide |
| 450 | 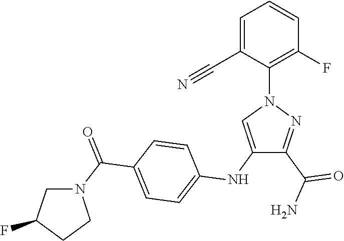 [SMILES: N#Cc1cccc(F)c1-n1cc(Nc2ccc(C(=O)N3CC[C@@H](F)C3)cc2)c(C(N)=O)n1] | (R)-1-(2-cyano-6-fluorophenyl)-4-((4- (3-fluoropyrrolidine-1- carbonyl)phenyl)amino)-1H-pyrazole- 3-carboxamide |
| 451 | 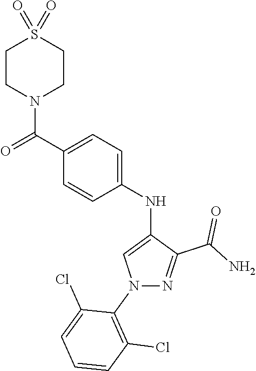 [SMILES: NC(=O)c1nn(-c2c(Cl)cccc2Cl)cc1Nc1ccc(C(=O)N2CCS(=O)(=O)CC2)cc1] | 1-(2,6-dichlorophenyl)-4-((4-(1,1- dioxidothiomorpholine-4- carbonyl)phenyl)amino)-1H-pyrazole- 3-carboxamide |
| 452 | 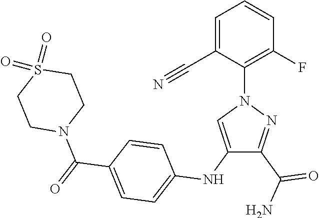 [SMILES: N#Cc1cccc(F)c1-n1cc(Nc2ccc(C(=O)N3CCS(=O)(=O)CC3)cc2)c(C(N)=O)n1] | 1-(2-cyano-6-fluorophenyl)-4-((4- (1,1-dioxidothiomorpholine-4- carbonyl)phenyl)amino)-1H-pyrazole- 3-carboxamide |
| 453 | 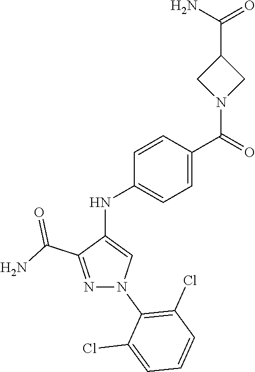 [SMILES: NC(=O)c1nn(-c2c(Cl)cccc2Cl)cc1Nc1ccc(C(=O)N2CC(C(N)=O)C2)cc1] | 4-[4-(3-Carbamoyl-azetidine-1- carbonyl)-phenylamino]-1-(2,6- dichloro-phenyl)-1H-pyrazole-3- carboxylic acid amide |
| 454 | 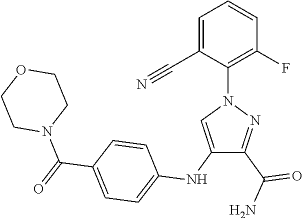 [SMILES: N#Cc1cccc(F)c1-n1cc(Nc2ccc(C(=O)N3CCOCC3)cc2)c(C(N)=O)n1] | 1-(2-cyano-6-fluorophenyl)-4-((4- (morpholine-4- carbonyl)phenyl)amino)-1H-pyrazole- 3-carboxamide |
| 455 | 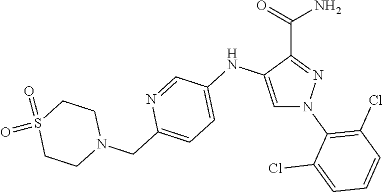 [SMILES: NC(=O)c1nn(-c2c(Cl)cccc2Cl)cc1Nc1ccc(CN2CCS(=O)(=O)CC2)nc1] | 1-(2,6-dichlorophenyl)-4-((6-((1,1- dioxidothiomorpholino)methyl) pyridin-3-yl)amino)-1H-pyrazole-3- carboxamide |
| 456 | 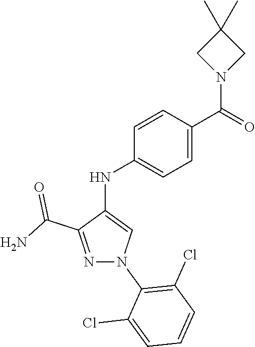 [SMILES: CC1(C)CN(C(=O)c2ccc(Nc3cn(-c4c(Cl)cccc4Cl)nc3C(N)=O)cc2)C1] | 1-(2,6-dichlorophenyl)-4-((4-(3,3- dimethylazetidine-1- carbonyl)phenyl)amino)-1H-pyrazole- 3-carboxamide |
| 457 | 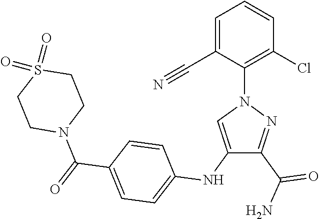 [SMILES: N#Cc1cccc(Cl)c1-n1cc(Nc2ccc(C(=O)N3CCS(=O)(=O)CC3)cc2)c(C(N)=O)n1] | 1-(2-chloro-6-cyanophenyl)-4-((4- (1,1-dioxidothiomorpholine-4- carbonyl)phenyl)amino)-1H-pyrazole- 3-carboxamide |
| 458 | 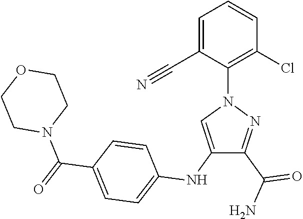 [SMILES: N#Cc1cccc(Cl)c1-n1cc(Nc2ccc(C(=O)N3CCOCC3)cc2)c(C(N)=O)n1] | 1-(2-chloro-6-cyanophenyl)-4-((4- (morpholine-4- carbonyl)phenyl)amino)-1H-pyrazole- 3-carboxamide |
| 459 | 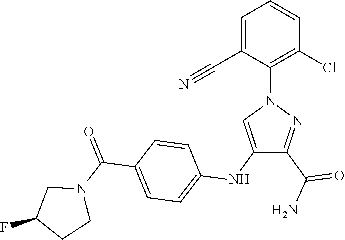 [SMILES: N#Cc1cccc(Cl)c1-n1cc(Nc2ccc(C(=O)N3CC[C@@H](F)C3)cc2)c(C(N)=O)n1] | 1-(2-Chloro-6-cyano-phenyl)-4-[4- ((R)-3-fluoro-pyrrolidine-1-carbonyl)- phenylamino]-1H-pyrazole-3- carboxylic acid amide |
| 460 | 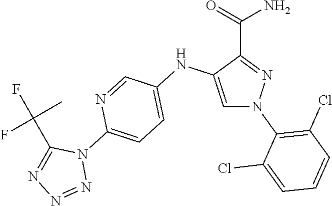 [SMILES: CC(F)(F)c1nnnn1-c1ccc(Nc2cn(-c3c(Cl)cccc3Cl)nc2C(N)=O)cn1] | 1-(2,6-dichlorophenyl)-4-((6-(5-(1,1- difluoroethyl)-1H-tetrazol-1- yl)pyridin-3-yl)amino)-1H-pyrazole- 3-carboxamide |
| 461 | 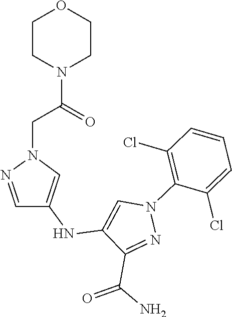 [SMILES: NC(=O)c1nn(-c2c(Cl)cccc2Cl)cc1Nc1cnn(CC(=O)N2CCOCC2)c1] | 1-(2,6-dichlorophenyl)-4-((1-(2- morpholino-2-oxoethyl)-1H-pyrazol- 4-yl)amino)-1H-pyrazole-3- carboxamide |
| 462 | 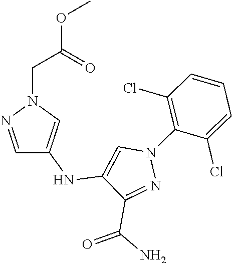 [SMILES: COC(=O)Cn1cc(Nc2cn(-c3c(Cl)cccc3Cl)nc2C(N)=O)cn1] | methyl_2-(4-((3-carbamoyl-1-(2,6- dichlorophenyl)-1H-pyrazol-4- yl)amino)-1H-pyrazol-1-yl)acetate |
| 463 | 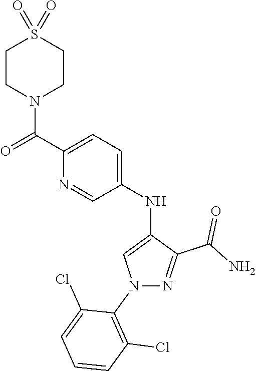 [SMILES: NC(=O)c1nn(-c2c(Cl)cccc2Cl)cc1Nc1ccc(C(=O)N2CCS(=O)(=O)CC2)nc1] | 1-(2,6-dichlorophenyl)-4-((6-(1,1- dioxidothiomorpholine-4- carbonyl)pyridin-3-yl)amino)-1H- pyrazole-3-carboxamide |
| 464 | 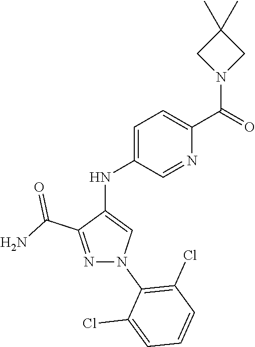 [SMILES: CC1(C)CN(C(=O)c2ccc(Nc3cn(-c4c(Cl)cccc4Cl)nc3C(N)=O)cn2)C1] | 1-(2,6-dichlorophenyl)-4-((6-(3,3- dimethylazetidine-1-carbonyl)pyridin- 3-yl)amino)-1H-pyrazole-3- carboxamide |
| 465 | 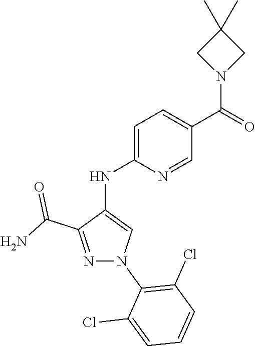 [SMILES: CC1(C)CN(C(=O)c2ccc(Nc3cn(-c4c(Cl)cccc4Cl)nc3C(N)=O)nc2)C1] | 1-(2,6-dichlorophenyl)-4-((5-(3,3- dimethylazetidine-1-carbonyl)pyridin- 2-yl)amino)-1H-pyrazole-3- carboxamide |
| 466 | 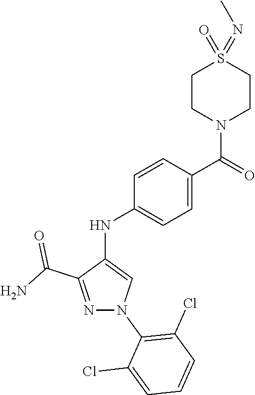 [SMILES: CN=S1(=O)CCN(C(=O)c2ccc(Nc3cn(-c4c(Cl)cccc4Cl)nc3C(N)=O)cc2)CC1] | 1-(2,6-dichlorophenyl)-4-((4-(1- (methylimino)-1- oxidothiomorpholine-4- carbonyl)phenyl)amino)-1H-pyrazole- 3-carboxamide |
| 467 | 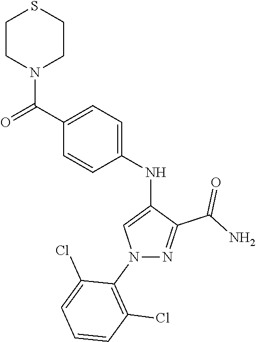 [SMILES: NC(=O)c1nn(-c2c(Cl)cccc2Cl)cc1Nc1ccc(C(=O)N2CCSCC2)cc1] | 1-(2,6-dichlorophenyl)-4-((4- (thiomorpholine-4- carbonyl)phenyl)amino)-1H-pyrazole- 3-carboxamide |
| 468 | 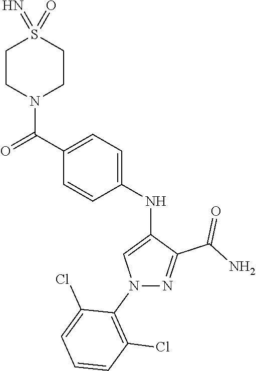 [SMILES: N=S1(=O)CCN(C(=O)c2ccc(Nc3cn(-c4c(Cl)cccc4Cl)nc3C(N)=O)cc2)CC1] | 1-(2,6-dichlorophenyl)-4-((4-(1- imino-1-oxidothiomorpholine-4- carbonyl)phenyl)amino)-1H-pyrazole- 3-carboxamide |
| 469 | 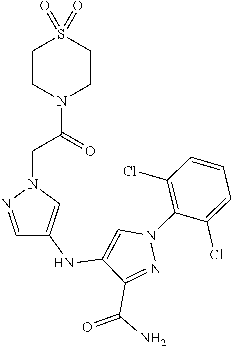 [SMILES: NC(=O)c1nn(-c2c(Cl)cccc2Cl)cc1Nc1cnn(CC(=O)N2CCS(=O)(=O)CC2)c1] | 1-(2,6-dichlorophenyl)-4-((1-(2-(1,1- dioxidothiomorpholino)-2-oxoethyl)- 1H-pyrazol-4-yl)amino)-1H-pyrazole- 3-carboxamide |
| 470 | 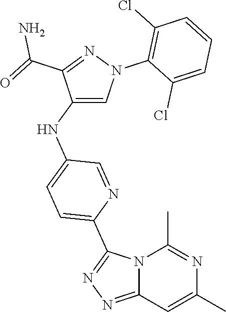 [SMILES: Cc1cc2nnc(-c3ccc(Nc4cn(-c5c(Cl)cccc5Cl)nc4C(N)=O)cn3)n2c(C)n1] | 1-(2,6-dichlorophenyl)-4-((6-(5,7- dimethyl-[1,2,4]triazolo[4,3- c]pyrimidin-3-yl)pyridin-3-yl)amino)- 1H-pyrazole-3-carboxamide |
| 471 | 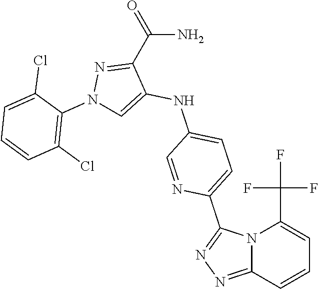 [SMILES: NC(=O)c1nn(-c2c(Cl)cccc2Cl)cc1Nc1ccc(-c2nnc3cccc(C(F)(F)F)n23)nc1] | 1-(2,6-dichlorophenyl)-4-((6-(5- (trifluoromethyl)-[1,2,4]triazolo[4,3- a]pyridin-3-yl)pyridin-3-yl)amino)- 1H-pyrazole-3-carboxamide |
| 472 | 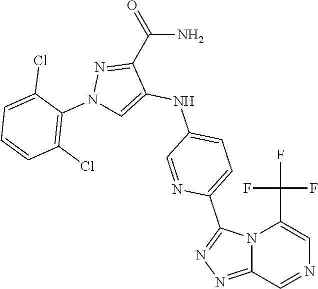 [SMILES: NC(=O)c1nn(-c2c(Cl)cccc2Cl)cc1Nc1ccc(-c2nnc3cncc(C(F)(F)F)n23)nc1] | 1-(2,6-dichlorophenyl)-4-((6-(5- (trifluoromethyl)-[1,2,4]triazolo[4,3- a]pyrazin-3-yl)pyridin-3-yl)amino)- 1H-pyrazole-3-carboxamide |
| 473 | 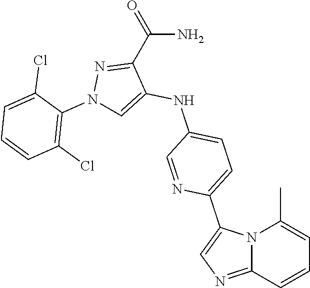 [SMILES: Cc1cccc2ncc(-c3ccc(Nc4cn(-c5c(Cl)cccc5Cl)nc4C(N)=O)cn3)n12] | 1-(2,6-dichlorophenyl)-4-((6-(5- methylimidazo[1,2-a]pyridin-3- yl)pyridin-3-yl)amino)-1H-pyrazole- 3-carboxamide |
| 474 | 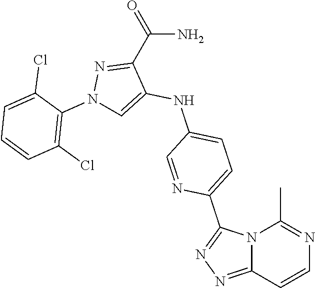 [SMILES: Cc1nccc2nnc(-c3ccc(Nc4cn(-c5c(Cl)cccc5Cl)nc4C(N)=O)cn3)n12] | 1-(2,6-dichlorophenyl)-4-((6-(5- methyl-[1,2,4]triazolo[4,3- c]pyrimidin-3-yl)pyridin-3-yl)amino)- 1H-pyrazole-3-carboxamide |
| 475 | 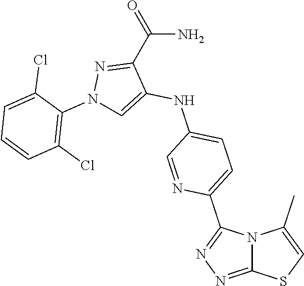 [SMILES: Cc1csc2nnc(-c3ccc(Nc4cn(-c5c(Cl)cccc5Cl)nc4C(N)=O)cn3)n12] | 1-(2,6-dichlorophenyl)-4-((6-(5- methylthiazolo[2,3-c][1,2,4]triazol-3- yl)pyridin-3-yl)amino)-1H-pyrazole- 3-carboxamide |
| 476 | 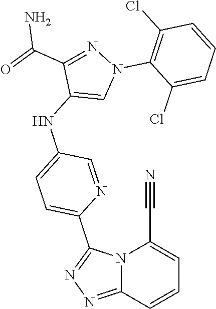 [SMILES: N#Cc1cccc2nnc(-c3ccc(Nc4cn(-c5c(Cl)cccc5Cl)nc4C(N)=O)cn3)n12] | 4-((6-(5-cyano-[1,2,4]triazolo[4,3- a]pyridin-3-yl)pyridin-3-yl)amino)-1- (2,6-dichlorophenyl)-1H-pyrazole-3- carboxamide |
| 477 | 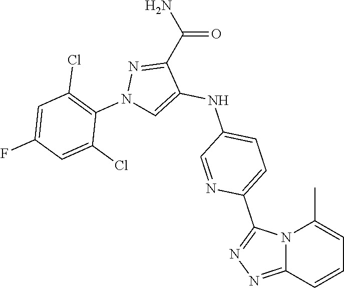 [SMILES: Cc1cccc2nnc(-c3ccc(Nc4cn(-c5c(Cl)cc(F)cc5Cl)nc4C(N)=O)cn3)n12] | 1-(2,6-dichloro-4-fluorophenyl)-4-((6- (5-methyl-[1,2,4]triazolo[4,3- a]pyridin-3-yl)pyridin-3-yl)amino)- 1H-pyrazole-3-carboxamide |
| 478 | | 4-((4-(3-carbamoylazetidine-1- |
| | | carbonyl)phenyl)amino)-1-(2,6- |
| | | dichlorophenyl)-1H-pyrazole-3- |
| | | carboxamide |

### Pharmaceutical Compositions

The present invention provides pharmaceutical compositions containing one or more compounds described above, or a pharmaceutically acceptable ester, prodrug, hydrate, solvate or salt of such a compound, optionally in combination with a pharmaceutically acceptable carrier. The invention further provides such compounds for the preparation of a medicament for the treatment of one or more diseases mentioned herein.

A pharmaceutical composition may contain one or more compounds of the invention in a therapeutically effective amount. A therapeutically effective amount of a compound in accordance with this invention means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage may be adjusted to the individual requirements in each particular case including the specific compound being administered, the route of administration, the condition being treated, as well as the patient being treated.

Compositions of the invention may include a vehicle for delivery of one or more compounds of the invention. For example, the composition may contain particles, such as nanoparticles, microparticles, liposomes, micelles, and virus particles.

Examples of pharmacologically acceptable salts of sufficiently basic compounds of the invention are salts of physiologically acceptable mineral acids like hydrochloric, hydrobromic, sulfuric and phosphoric acid; or salts of organic acids like methanesulfonic, p-toluenesulfonic, lactic, acetic, trifluoroacetic, citric, succinic, fumaric, maleic and salicylic acid. Further, a sufficiently acidic compound of the invention may form alkali or alkaline earth metal salts, for example sodium, potassium, lithium, calcium or magnesium salts; ammonium salts; or organic base salts, for example methylamine, dimethylamine, trimethylamine, triethylamine, ethylenediamine, ethanolamine, choline hydroxide, meglumin, piperidine, morpholine, tris-(2-hydroxyethyl)amine, lysine or arginine salts; all of which are also further examples of salts of the invention. Compounds of the invention may be solvated, especially hydrated. The solvation may occur during the process of production or as a consequence of the hygroscopic nature of the initially water free compounds of the invention. The solvates and/or hydrates may e.g. be present in solid or liquid form.

It should be appreciated that certain compounds of the invention may have tautomeric forms from which only one might be specifically mentioned or depicted in the following description, different geometrical isomers (which are usually denoted as cis/trans isomers or more generally as (E) and (Z) isomers) or different optical isomers as a result of one or more chiral carbon atoms (which are usually nomenclatured under the Cahn-Ingold-Prelog or R/S system). All these tautomeric forms, geometrical or optical isomers (as well as racemates and diastereomers) and polymorphous forms are included in the invention. Since the compounds of the invention may contain asymmetric C-atoms, they may be present either as achiral compounds, mixtures of diastereomers, mixtures of enantiomers or as optically pure compounds. The present invention comprises both all pure enantiomers and all pure diastereomers, and also the mixtures thereof in any mixing ratio.

According to a further embodiment of the present invention, one or more hydrogen atoms of the compounds of the present invention may be replaced by deuterium. Deuterium modification improves the metabolic properties of a drug with little or no change in its intrinsic pharmacology. Deuterium substitution at specific molecular positions improves metabolic stability, reduces formation of toxic metabolites and/or increases the formation of desired active metabolites. Accordingly, the present invention also encompasses the partially and fully deuterated compounds of the invention. The term hydrogen also encompasses deuterium.

The therapeutic use of compounds according to the invention, their pharmacologically acceptable salts, solvates and hydrates, respectively, as well as formulations and pharmaceutical compositions also lie within the scope of the present invention. The pharmaceutical compositions according to the present invention may comprise at least one compound of the invention as an active ingredient and, optionally, carrier substances and/or adjuvants.

The present invention also relates to prodrugs which are composed of a compound of the invention and at least one pharmacologically acceptable protective group which will be cleaved under physiological conditions, such as an alkoxy-, arylalkyloxy-, acyl-, acyloxymethyl group (e.g. pivaloyloxymethyl), an 2-alkyl-, 2-aryl- or 2-arylalkyl oxycarbonyl-2-alkylidene ethyl group or an acyloxy group as defined herein, e.g. ethoxy, benzyloxy, acetyl or acetyloxy or, especially for a compound of the invention, carrying a hydroxy group (—OH): a sulfate, a phosphate (—OPO₃ or —OCH₂OPO₃) or an ester of an amino acid. For example, compositions may contain pro-drugs of the hydroxy group of a compound of the invention.

As used herein, the term pharmaceutically acceptable ester especially refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The present invention also relates to a prodrug, a biohydrolyzable ester, a biohydrolyzable amide, a polymorph, tautomer, stereoisomer, metabolite, N-oxide, biohydrolyzable carbamate, biohydrolyzable ether, physiologically functional derivative, atropisomer, or in vivo-hydrolysable precursor, diastereomer or mixture of diastereomers, chemically protected form, affinity reagent, complex, chelate and a stereoisomer of the compounds of the invention.

As mentioned above, therapeutically useful agents that contain compounds of the invention, their solvates, salts or formulations are also comprised in the scope of the present invention. In general, compounds of the invention will be administered by using the known and acceptable modes known in the art, either alone or in combination with any other therapeutic agent.

For oral administration such therapeutically useful agents can be administered by one of the following routes: oral, e.g. as tablets, dragees, coated tablets, pills, semisolids, soft or hard capsules, for example soft and hard gelatin capsules, aqueous or oily solutions, emulsions, suspensions or syrups, parenteral including intravenous, intramuscular and subcutaneous injection, e.g. as an injectable solution or suspension, rectal as suppositories, by inhalation or insufflation, e.g. as a powder formulation, as microcrystals or as a spray (e.g. liquid aerosol), transdermal, for example via an transdermal delivery system (TDS) such as a plaster containing the active ingredient or intranasal. For the production of such tablets, pills, semisolids, coated tablets, dragees and hard, e.g. gelatin, capsules, the therapeutically useful product may be mixed with pharmaceutically inert, inorganic or organic excipients as are e.g. lactose, sucrose, glucose, gelatin, malt, silica gel, starch or derivatives thereof, talc, stearinic acid or their salts, dried skim milk, and the like. For the production of soft capsules, one may use excipients as are e.g. vegetable, petroleum, animal or synthetic oils, wax, fat, polyols. For the production of liquid solutions, emulsions or suspensions or syrups one may use as excipients e.g. water, alcohols, aqueous saline, aqueous dextrose, polyols, glycerin, lipids, phospholipids, cyclodextrins, vegetable, petroleum, animal or synthetic oils. Particularly useful are lipids, such as phospholipids (e.g., natural origin and/or with a particle size between 300 to 350 nm) in phosphate buffered saline (pH=7 to 8, e.g., 7.4). For suppositories one may use excipients as are e.g. vegetable, petroleum, animal or synthetic oils, wax, fat and polyols. For aerosol formulations one may use compressed gases suitable for this purpose, as are e.g. oxygen, nitrogen and carbon dioxide. The pharmaceutically useful agents may also contain additives for conservation, stabilization, e.g. UV stabilizers, emulsifiers, sweetener, aromatizers, salts to change the osmotic pressure, buffers, coating additives and antioxidants.

In general, in the case of oral or parenteral administration to adult humans weighing approximately 80 kg, a daily dosage of about 10 mg to about 10,000 mg, or from about 20 mg to about 1,000 mg, should be appropriate, although the upper limit may be exceeded when indicated. The daily dosage can be administered as a single dose or in divided doses, or for parenteral administration, it may be given as continuous infusion or subcutaneous injection.

### Methods of Making Compounds

The invention also provides methods of making compounds of the invention, such as those described above. Synthesis schemes for making specific compounds of Formula (I) are provided in the Examples below.

### Methods of Treating Conditions

The compounds and compositions of the invention modulate activity of one or more protein kinases. The compounds and compositions may inhibit, activate, or otherwise alter kinase activity. Consequently, the compounds and compositions may be used to diagnose, treat, or prevent a condition, such as a disease, disorder, or other condition for which modulation of kinase activity provides therapeutic benefit.

Diseases, disorders, and conditions that can be diagnosed and/or treated using compositions and methods of the invention include (but are not restricted to) those associated with aberrant activity, e.g., increased activity or decreased activity, of one or more kinases. The kinase may be a serine-threonine kinase or a tyrosine kinase, e.g., a receptor tyrosine kinase or non-receptor tyrosine kinase. The kinase may be a member of the JAK family. For example and without limitation, the kinase may be leucine-rich repeat kinase 2 (LRRK2), NUAK family SNF1-like kinase 1 (NUAK1, also known as AMPK-related protein kinase 5 or ARK5), JAK1, JAK2, or non-receptor tyrosine-protein kinase TYK2 (TYK2), including mutants of any of the aforementioned kinases.

The disease, disorder, or condition may be associated with aberrant LRRK2 activity, such as Alzheimer's disease, Crohn's disease, inflammatory bowel disease, an inflammatory disease, leprosy, neurodegenerative diseases, a non-skin cancer, or Parkinson's disease, including familial Parkinson's disease, sporadic Parkinson's disease, late-onset Parkinson's disease (PD), and type 8 Parkinson's disease.

The disease, disorder, or condition may be associated with aberrant NUAK1 activity, such as cancer, e.g., colorectal cancer, stomach cancer, endometrial cancer, or multiple myeloma, diabetes, fibrosis, a neurodegenerative disease, or omphalocele.

The disease, disorder, or condition may be associated with aberrant TYK2 activity, such as autoimmune disorders, Crohn's disease, hyperimmunoglobulin E syndrome, inflammatory bowel disease, multiple sclerosis (MS), psoriasis, rheumatoid arthritis, systemic lupus erythematosus (SLE), type 1 diabetes (T1D), or ulcerative colitis.

The disease, disorder, or condition may be or include a respiratory tract/obstructive airways disease or disorder, such as rhinorrhea, tracheal constriction, airway contraction, acute-, allergic, atrophic rhinitis or chronic rhinitis (such as rhinitis caseosa, hypertrophic rhinitis, rhinitis purulenta, rhinitis sicca), rhinitis medicamentosa, membranous rhinitis (including croupous, fibrinous and pseudomembranous rhinitis), scrofulous rhinitis, perennial allergic rhinitis, seasonal rhinitis (including rhinitis nervosa (hay fever) and vasomotor rhinitis), pollinosis, asthma (such as bronchial, atopic, allergic, intrinsic, extrinsic, exercise-induced, cold air-induced, occupational, bacterial infection-induced, and dust asthma particularly chronic or inveterate asthma (e.g. late asthma and airways hyper-responsiveness)), bronchitis (including chronic, acute, arachidic, catarrhal, croupus, phthinoid and eosinophilic bronchitis), cardiobronchitis, pneumoconiosis, chronic inflammatory disease of the lung which result in interstitial fibrosis, such as interstitial lung disease (ILD) (e.g., idiopathic pulmonary fibrosis, or ILD associated with rheumatoid arthritis, or other autoimmune conditions), acute lung injury (ALI), adult respiratory distress syndrome (ARDS), chronic obstructive pulmonary, airways or lung disease (CORD, COAD, COLD or COPD, such as irreversible COPD), chronic sinusitis, conjunctivitis (e.g. allergic conjunctivitis), cystic fibrosis, extrinsic allergic alveolitis (like farmer's lung and related diseases), fibroid lung, hypersensitivity lung diseases, hypersensitivity pneumonitis, idiopathic interstitial pneumonia, nasal congestion, nasal polyposis, otitis media, and cough (chronic cough associated with inflammation or iatrogenic induced), pleurisy, pulmonary congestion, emphysema, bronchiectasis, sarcoidosis, lung fibrosis, including cryptogenic fibrosing alveolitis, fibrosis complicating anti-neoplastic therapy and chronic infection, including tuberculosis and aspergillosis and other fungal infections, vasculitic and thrombotic disorders of the lung vasculature, and pulmonary hypertension, acute viral infection including the common cold, and infection due to respiratory syncytial virus, influenza, coronavirus (including SARS) and adenovirus, allergic bronchopulmonary mycosis, emphysema, diffuse panbronchiolitis, systemic anaphylaxis or hypersensitivity responses, drug allergies (e.g., to penicillin, cephalosporins), insect sting allergies, and food related allergies which may have effects remote from the gut (such as migraine, rhinitis and eczema), anaphylactic shock, or vascular spasms.

The disease, disorder, or condition may be or include a bone and joint related disease or disorder, such as osteoporosis, arthritis (including rheumatic, infectious, autoimmune, chronic, malignant), seronegative spondyloarthropathies (such as ankylosing spondylitis, rheumatoid spondylitis, psoriatic arthritis, enthesopathy, Bechet's disease, Marie-Strumpell arthritis, arthritis of inflammatory bowel disease, and Reiter's disease), systemic sclerosis, osteoarthritis, osteoarthrosis, both primary and secondary to e.g. congenital hip dysplasia, cervical and lumbar spondylitis, and low back and neck pain, Still's disease, reactive arthritis and undifferentiated spondarthropathy, septic arthritis and other infection-related arthropathies and bone disorders such as tuberculosis, including Pott's disease and Poncet's syndrome, acute and chronic crystal-induced synovitis including urate gout, calcium pyrophosphate deposition disease, and calcium apatite related tendon, bursar and synovial inflammation, primary and secondary Sjogren's syndrome, systemic sclerosis and limited scleroderma, mixed connective tissue disease, and undifferentiated connective tissue disease, inflammatory myopathies including, polymyalgia rheumatica, juvenile arthritis including idiopathic inflammatory arthritides of whatever joint distribution and associated syndromes, other joint disease (such as intervertebral disc degeneration or temporomandibular joint degeneration), rheumatic fever and its systemic complications, vasculitides including giant cell arteritis, Takayasu's arteritis, polyarteritis nodosa, microscopic polyarteritis, and vasculitides to associated with viral infection, hypersensitivity reactions, cryoglobulins, paraproteins, low back pain, Familial Mediterranean fever, Muckle-Wells syndrome, and Familial Hibenian Fever, Kikuchi disease, drug-induced arthralgias, tendonititides, polychondritis, and myopathies, osteoporosis, osteomalacia like osteoporosis, osteopenia, osteogenesis imperfects, osteopetrosis, osteofibrosis, osteonecrosis, Paget's disease of bone, hypophosphatemia, Felty's syndrome, Still's disease, slack of artificial joint implant, sprain or strain of muscle or joint, tendinitis, fasciitis, periarthritis humeroscapularis, cervico-omo-brachial syndrome, or tenosynovitis.

The disease, disorder, or condition may be or include a skin or eye related disease or disorder, such as glaucoma, ocular hypertension, cataract, retinal detachment, psoriasis (including psoriasis vulgaris, pustular psoriasis, arthritic psoriasis, erythroderma psoriaticum), palmoplantar pustulosis, xerodoma, eczematous diseases (like atopic dermatitis, ultraviolet radiation dermatitis, contact dermatitis, and seborrheic dermatitis), phytodermatitis, photodermatitis, cutaneous eosinophilias, chronic skin ulcers, cutaneous lupus erythematosus, contact hypersensitivity/allergic contact dermatitis (including sensitivity to poison ivy, sumac, or oak), and eosinophilic folliculitis (Ofuji's disease), pruritus, drug eruptions, urticaria (acute or chronic, allergic or non-allergic), acne, erythema, dermatitis herpetiformis, scleroderma, vitiligo, lichen planus, lichen sclerosus et atrophica, pyodenna gangrenosum, skin sarcoid, pemphigus, ocular pemphigus, pemphigoid, epidermolysis bullosa, angioedema, vasculitides, toxic erythemas, cutaneous eosinophilias, alopecia areata, male-pattern baldness, Sweet's syndrome, Stevens-Johnson syndrome, Weber-Christian syndrome, erythema multiforme, cellulitis, both, infective and non infective, panniculitis, cutaneous Lymphomas, non, melanoma skin cancer and other dysplastic lesions, blepharitis, iritis, anterior and posterior uveitis, choroiditis, autoimmune, degenerative or inflammatory disorders affecting the retina, ophthalmitis including sympathetic ophthalmitis, sarcoidosis, xerosis infections including viral, fungal, and bacterial, allergic conjunctivitis, increased fibrosis, keloids, keloplasty, post surgical scars, epidermolysis bullosa, dry eye, ocular inflammation, allergic conjunctivitis, vernal conjunctivitis, vernal keratoconjunctivitis, and giant papillary conjunctivitis, ocular angiogenesis, cornea damage and scar, all forms of macular degeneration, macular edema, macular dystrophy, abnormal wound healing, scleritis, episcleritis, pachydermia, peripheral ulcerative keratitis, fungal keratitis, herpetic keratitis, invasive aspergillosis; conical cornea, dystorphia epithelialis comeae, or severe intraocular inflammation.

The disease, disorder, or condition may be or include a gastrointestinal tract and abdominal related disease or disorder, such as celiac/coeliac disease (e.g. celiac sprue), cholecystitis, enteritis (including infectious, ischemic, radiation, drug-induced, and eosinophilic gastroenteritis), eosinophilic esophagitis, eosinophilic gastrointestinal inflammation, allergen induced diarrhea, enteropathy associated with seronegative arthropathies, gastritis, autoimmune atrophic gastritis, ischemic bowel disease, inflammatory bowel disease (Crohn's disease and ulcerative colitis), colitis, Mooren's ulcer, irritable bowel syndrome, necrotizing enterocolitis, gut ischemia, glossitis, gingivitis, periodontitis, oesophagitis, including reflex, proctitis, fibrosis and cirrhosis of the liver, pancreatitis, both acute and chronic, pancreatic fibrosis, pancreatic sclerosis, pancreatolithiasis, hepatic cirrhosis, hepatitis (congestive, autoimmune, acute, fulminant, chronic, drug-induced, alcoholic, lupoid, steatohepatitis and chronic viral), fatty liver, primary biliary cirrhosis, hepatic porphyria, and gastrointestinal related allergic disorders, spastic colon, diverticulitis, gastroenteric bleeding, Behcet's disease; partial liver resection, acute liver necrosis (e.g. necrosis caused by toxins, viral hepatitis, shock or anoxia), or hemolytic uremic syndrome.

The disease, disorder, or condition may be or include a hematological disease or disorder, such as anemias, coagulation, myeloproliferative disorders, hemorrhagic disorders, leukopenia, eosinophilic disorders, leukemias (e.g. myelogenous, lymphomas, plasma cell dyscrasias, disorders of the spleen, Band's disease, hemophilia, purpura (including idiopathic thrombocytopenic purpura), or Wiskott-Aldrich syndrome.

The disease, disorder, or condition may be or include a metabolic disease or disorder, such as obesity, amyloidosis, disturbances of the amino and acid metabolism like branched chain disease, hyperaminoacidemia, hyperaminoaciduria, disturbances of the metabolism of urea, hyperammonemia, mucopolysaccharidoses e.g. Maroteaux-Lamy syndrome, storage disease like glycogen storage diseases and lipid storage diseases, glycogenosis I diseases like Cori's disease, malabsorption diseases like intestinal carbohydrate malabsorption, oligosaccharidase deficiency like maltase-, lactase-, sucrase-insufficiency, disorders of the metabolism of fructose, disorders of the metabolism of galactose, galactosaemia, disturbances of carbohydrate utilization like diabetes, hypoglycemia, disturbances of pyruvate metabolism, hypolipidemia, hypolipoproteinemia, hyperlipidemia, hyperlipoproteinemia, carnitine or carnitine acyltransferase deficiency, disturbances of the porphyrin metabolism, porphyrins, disturbances of the purine metabolism, lysosomal diseases, metabolic diseases of nerves and nervous systems like gangliosidoses, sphingolipidoses, sulfatidoses, leucodystrophies, or Lesch Nyhan syndrome.

The disease, disorder, or condition may be or include a cerebellar dysfunction or disturbance of brain metabolism, such as dementia, Alzheimer's disease, Huntington's chores, Parkinson's disease, Pick's disease, toxic encephalopathy, demyelinating neuropathies like inflammatory neuropathy, Guillain-Barre syndrome; Meniere's disease and radiculopathy, primary and secondary metabolic disorders associated with hormonal defects like any disorder stemming from either an hyperfunction or hypofunction of some hormone-secreting endocrine gland and any combination thereof. Sipple's syndrome, pituitary gland dysfunction and its effects on other endocrine glands, such as the thyroid, adrenals, ovaries, and testes, acromegaly, hyper- and hypothyroidism, euthyroid goiter, euthyroid sick syndrome, thyroiditis, and thyroid cancer, over or underproduction of the adrenal steroid hormones, adrenogenital syndrome, Cushing's syndrome, Addison's disease of the adrenal cortex, Addison's pernicious anemia, primary and secondary aldosteronism, diabetes insipidus, diabetes mellitus, carcinoid syndrome, disturbances caused by the dysfunction of the parathyroid glands, pancreatic islet cell dysfunction, diabetes, disturbances of the endocrine system of the female like estrogen deficiency, resistant ovary syndrome; muscle weakness, myotonia. Duchenne's and other muscular dystrophies, dystrophia myotonica of Steinert, mitochondrial myopathies like disturbances of the catabolic metabolism in the muscle, carbohydrate and lipid storage myopathies, glycogenoses, myoglobinuria, malignant hyperthermia, polymyalgia rheumatics, dermatomyositis, multiple myositis, primary myocardial disease, cardiomyopathy; disorders of the ectoderm, neurofibromatosis, scleroderma and polyar teritis, Louis-Bar syndrome, von Hippel-Lindau disease, Sturge-Weber syndrome, tuberous sclerosis, amyloidosis, porphyria; sexual dysfunction of the male and female; confused states and seizures due to inappropriate secretion of antidiuretic hormone from the pituitary gland, Liddle's syndrome, Bartter's syndrome, Fanconi's I syndrome, or renal electrolyte wasting.

The disease, disorder, or condition may be or include a transplant rejection related condition, such as acute and chronic allograft rejection following solid organ transplant, for example, transplantation of kidney, heart, liver, lung, and cornea, chronic graft versus host disease, skin graft rejection, and bone marrow transplant rejection, or immunosuppression.

The disease, disorder, or condition may be or include a genitourinary related condition, such as nephritis (interstitial, acute interstitial (allergic), and glomerulonephritis), nephrotic syndrome, cystitis including acute and chronic (interstitial) cystitis and Hunner's ulcer, acute and chronic urethritis, prostatitis, epididymitis, oophoritis, salpingitis, vulvo vaginitis, vulvovaginal candidiasis, Peyronie's disease, and erectile dysfunction, renal disease, renal fibrosis, nephropyelitis, secondary contracted kidney, steroid dependent and steroid-resistant nephrosis, or Goodpasture's syndrome.

The disease, disorder, or condition may be or include a CNS related disease or disorder, such as neurodegenerative diseases, Alzheimer's disease and other cementing disorders including CJD and nvCJD, amyloidosis, and other demyelinating syndromes, cerebral atherosclerosis and vasculitis, temporal arteritis, myasthenia gravis, acute and chronic so pain (acute, intermittent or persistent, whether of central or peripheral origin) including post-operative, visceral pain, headache, migraine, neuralgia (including trigeminal), atypical facial pain, joint and bone pain, pain arising from cancer and tumor invasion, neuropathic pain syndromes including diabetic, post-herpetic, and HIV-associated neuropathies, neurosarcoidosis, to brain injuries, cerebrovascular diseases and their consequences, Parkinson's disease, corticobasal degeneration, motor neuron disease, dementia, including ALS (Amyotrophic-lateral sclerosis), multiple sclerosis, traumatic brain injury, stroke, post-stroke, post-traumatic brain injury, and small-vessel cerebrovascular disease, dementias, vascular dementia, dementia with Lewy bodies, frontotemporal dementia and Parkinsonism linked 1 to chromosome 17, frontotemporal dementias, including Pick's disease, progressive supranuclear palsy, corticobasal degeneration, Huntington's disease, thalamic degeneration, HIV dementia, schizophrenia with dementia, and Korsakoffs psychosis, within the meaning of the definition are also considered to be CNS disorders central and peripheral nervous system complications of malignant, infectious or autoimmune processes, algesia, cerebral infarction, attack, cerebral ischemia, head injury, spinal cord injury, myelopathic muscular atrophy, Shy-Drager syndrome, Reye's syndrome, progressive multifocal leukoencephalopathy, normal pressure hydrocephalus, sclerosing panencephalitis, frontal lobe type dementia, acute anterior poliomyelitis (poliomyelitis), poliomyelitis neurosis, viral encephalitis, allergic encephalomyelitis, epileptic encephalopathies, Creutzfeldt-Jakob disease, Kuru disease, bovine spongiform encephalopathy (mad cow disease), scrapie, epilepsy, cerebral amyloid angiopathy, depression, mania, manic-depressive psychosis, hereditary cerebellar ataxia, peripheral neuropathy, Nasu-Hakola syndrome, or Machado-Joseph disease.

The disease, disorder, or condition may be or include an inflammatory or immunological disease or disorder, such as general inflammation (of the ocular, nasal, pulmonary, and gastrointestinal passages), mastocytosis/mast cell disorders (cutaneous, systemic, mast cell activation syndrome, and pediatric mast cell diseases), mastitis (mammary gland), vaginitis, vasculitis (e.g., necrotizing, cutaneous, and hypersensitivity vasculitis), Wegener granulamatosis, myyositis (including polymyositis, dermatomyositis), basophil related diseases including basophilic leukemia and basophilic leukocytosis, and eosinophil related diseases such as Churg-Strauss syndrome, eosinophilic granuloma, lupus erythematosus (such as, systemic lupus erythematosus, subacute cutaneous lupus erythematosus, and discoid lupus erythematosus), chronic thyroiditis, Hashimoto's thyroiditis, Grave's disease, type I diabetes, complications arising from diabetes mellitus, other immune disorders, eosinophilia fasciitis, hyper IgE syndrome, Addison's disease, antiphospholipid syndrome, immunodeficiency disease, acquired immune deficiency syndrome (AIDS), leprosy, Sezary syndrome, paraneoplastic syndromes, and other autoimmune disorders, fervescence, myositis, nervous diseases selected from multiple myositis, bursitis, Evans syndrome, leukotriene B4-mediated diseases, idiopathic hypoparathyroidism, nephrotic syndrome lupus, or immunosuppression.

The disease, disorder, or condition may be or include a cardiovascular disease or disorder, such as congestive heart failure, myocardial infarction, ischemic diseases of the heart, all kinds of atrial and ventricular arrhythmias, hypertension, cerebral trauma, occlusive vascular disease, stroke, cerebrovascular disorder, atherosclerosis, restenosis, affecting the coronary and peripheral is circulation, pericarditis, myocarditis, inflammatory and auto-immune cardiomyopathies including myocardial sarcoid, endocarditis, valvulitis, and aortitis including infective (e.g. syphilitic), hypertensive vascular diseases, peripheral vascular diseases, and atherosclerosis, vasculitides, disorders of the proximal and peripheral veins including phlebitis and thrombosis, including deep vein thrombosis and complications of varicose veins, aortic aneurism, periarteritis nodosa, cardiac fibrosis, post-myocardial infarction, idiopathic cardiomyopathy, or angioplasty.

The disease, disorder, or condition may be or include an oncological disease or disorder, such as common cancers (prostate, breast, lung, ovarian, pancreatic, bowel and colon, abdomen, stomach (and any other digestive system cancers), liver, pancreas, peritoneum, endocrine glands (adrenal, parathyroid, pituitary, testicles, ovary, thymus, thyroid), eye, head, neck, nervous system (central and peripheral), lymphatic system, blood, pelvic, skin, bone, soft tissue, spleen, thoracic, urogenital, and brain tumors), breast cancer, genitourinary cancer, lung cancer, gastrointestinal cancer, epidermoid cancer, melanoma, ovarian cancer, pancreas cancer, neuroblastoma, malignancies affecting the bone marrow (including the leukemias) and lymphoproliferative systems, such as Hodgkin's and non-Hodgkin's lymphoma, B-cell lymphoma, follicular lymphoma, metastatic disease and tumor recurrences, and paraneoplastic syndromes, as well as hypergammaglobulinemia, lymphoproliferative diseases, disorders, and/or conditions, paraproteinemias, purpura (including idiopathic thrombocytopenic purpura), Waldenstron's Macroglobulinemia, Gaucher's Disease, histiocytosis, retinoblastoma and any other hyperproliferative disease, sarcomata, cachexia, tumor growth, tumor invasion, metastasis, AIDS-related lymphomas, malignant immunoproliferative diseases, multiple myeloma and malignant plasma cell neoplasms, lymphoid leukemia, acute or chronic myeloid leukemia, acute or chronic lymphocytic leukemia, monocytic leukemia, other leukemias of specified cell type, leukemia of unspecified cell type, other and unspecified malignant neoplasms of lymphoid, hematopoietic and related tissues, for example diffuse large cell lymphoma, T-cell lymphoma or cutaneous T-cell lymphoma). Myeloid cancer includes e.g. acute or chronic myeloid leukemia, or keratoleukoma.

The disease, disorder, or condition may be or include another disease or disorder, such as pain, migraine, sleep disorders, fever, sepsis, idiopathic thrombocytopenia purpura, post-operative adhesions, flushing, ischemic/reperfusion injury in the heart, brain, peripheral limbs, bacterial infection, viral infection, fungal infection, thrombosis, endotoxin shock, septic shock, thermal regulation including fever, Raynaud's disease, gangrene, diseases requiring anti-coagulation therapy, congestive heart failure, mucus secretion disorders, pulmonary hypotension, prostanoid-induced smooth muscle contract associated with dysmenorrhea and premature labor, premature delivery, reperfusion injury, bum, thermal injury, hemorrhage or traumatic shock, menstrual pain, menstrual cramp, dysmenorrhea, periodontosis, rickettsial infectious disease, protozoal disease, reproduction disease, toothache, pain after tooth extraction, Herpes zoster, Herpes simplex, retroperitoneal fibrosis, or various radiation injuries.

In certain embodiments, the disease is selected from the group consisting of an inflammatory disease, an autoimmune disease, an allergic disorder, and an ocular disorder. In certain embodiments, the disease is selected from the group consisting of pruritus, eczema, asthma, rhinitis, dry eye, ocular inflammation, allergic conjunctivitis, vernal conjunctivitis, vernal keratoconjunctivitis, giant papillary conjunctivitis, fungal keratitis and uveitis.

The method may include modulating the activity of one or more kinases in a subject, such as any of the kinase described above. The method may include inhibiting a kinase. The method may include activating, e.g., stimulating or enhancing the activity of, a kinase. The method may include modulating activity of a single kinase or preferentially modulating activity of a specific kinase over others. The method may include modulating activity of multiple kinases or preferentially modulating activity of two more specific kinases over others.

The method may include providing a compound of the invention. The method may include providing multiple compounds of the invention.

The method may include contacting cells containing a kinase with one or more compounds of the invention. For example and without limitation, contacting a cell with a compound may include exposing a cell to a compound, e.g., in a formulation, such as any of those described above; delivering a compound inside a cell; providing a compound to a subject and allowing a cell in the subject to become exposed to the compound. Contacting may be performed in vivo or in vitro. In vitro contact may include exposure of cells or tissue isolated from a subject. The method may include contacting cells with a single compound of the invention. The method may include contact cells with multiple compounds of the invention.

The method may include administration of a composition to a subject. The compositions may be provided by any suitable route of administration. For example and without limitation, the compositions may be administered buccally, by injection, dermally, enterally, intraarterially, intravenously, intranasally, e.g., by inhalation, intraocularly, orally, parenterally, pulmonarily, rectally, subcutaneously, systemically, topically, e.g., to the skin or eye, transdermally, or with or on an implantable medical device (e.g., stent or drug-eluting stent or balloon equivalents).

The method may include using a composition of the invention to diagnose a disease, disorder, or condition in a subject. For example, a radiolabeled form of a compound may be used a tracer in positron emission tomography (PET) to identify anatomical locations of aberrant kinase activity. PET is known in the art and described in, for example, Wadsak Wolfgang, Mitterhauser Markus (2010), “Basics and principles of radiopharmaceuticals for PET/CT”, European Journal of Radiology, 73 (3): 461-469. doi:10.1016/j.ejrad.2009.12.022; Bailey, D. L; D. W. Townsend; P. E. Valk; M. N. Maisey (2005), Positron Emission Tomography: Basic Sciences. Secaucus, NJ: Springer-Verlag, ISBN 1-85233-798-2; and Carlson, Neil (Jan. 22, 2012). Physiology of Behavior. Methods and Strategies of Research, 11th edition, Pearson, p. 151, ISBN 0205239390, the contents of each of which are incorporated herein by reference. The invention may include administering one or more compositions of the invention for both diagnostic and therapeutic purposes.

### EXAMPLES

### Example 1

The following methods were used in synthesis of compounds described herein.

Flash chromatography: Flash chromatography was performed on a Biotage Isolera® or Selekt® system using SNAP or SFÄR silica cartridges and ethyl acetate/cyclohexane/methanol or dichloromethane/methanol gradients as eluent.

Microwave conditions: Reactions under microwave conditions are performed in a Biotage Initiator® microwave system.

SEMIprep reversed phase chromatography: The following instrumentation was used for SEMIprep reversed phase chromatography: 2× Varian PrepStar SD-1, 1× Dionex P580 Pump 1 Channel (MakeUP I), 1× Dionex AXP-MS (MakeUP II), 1× Dionex MSQ, 1× Dionex UVD 340V—Prep Flow Cell, Gilson 215 Liquid Handler, SunFire Prep C18 OBD 5 μm, 19×50 mm column, 1×G7159B 1290 Infinity II Preparative Open-Bed Sampler/Collector, 1× G7161B 1290 Infinity II Preparative Binary Pump, 1×G7111B 1260 Infinity II Quaternary Pump (Modifier), 1×G7111B 1260 Infinity II Quaternary Pump (Analyltic/MakeUp), 1×G7165A 1260 Infinity II Multiple Wavelength Detector incl. Flow Cell (Product #G1315-60022, Serial #DE185H6157, Path Length 10.00 mm, Volume 13.00 μl), 1×G7170B 1290 Infinity II MS Flow Modulator, 1×G6125B MSD 6100 Series Single Quadrupole incl. G1948B Electrospray Interface, and 3×G1170A 1290 Infinity Valve Drive (14 Ports, 6 Positions Valve Head for Analytic Column Selection; 14 Ports, 6 Positions Valve Head for Preparative Column Selection; 14 Ports, 2 Positions Valve Head for Analytic/Preparative Mode Selection).

Preparative columns: Waters SunFire Prep C18 5 μm OBD 30×100 mm, #186002572, Waters Atlantis T3 Prep 5 μm OBD 30×100 mm, #186003702, and Waters XSelect CSH Prep C18 5 μm OBD 30×100 mm, #186005425

Analytical columns: Waters SunFire C18 2.5 μm 3.0×75 mm, #186005636, Waters Atlantis T3 3 μm 3.0×75 mm, #186005653, and Waters XSelect CSH C18 2.5 μm 3.0×75 mm, #186006106.

Typical chromatography conditions are as follows:

Column flow was 30 mL/min, Solvent A was methanol containing 0.3% acetic acid, and Solvent B was water containing 0.3% acetic acid. Typical times and relative volumes of Solvent and Solvent B are shown in Table 2.

TABLE 2

| Time (min) | Solv. A | Solv. B |
|---|---|---|
| 0.0 | 30.00 | 70.00 |
| 10.0 | 100.00 | 0.00 |
| 14.0 | 100.00 | 0.00 |
| 14.4 | 30.00 | 70.00 |
| 16.4 | 30.00 | 70.00 |

Typical preparative method: Column flow was 60 mL/min, Solvent A was acetonitrile, and Solvent B was water. Preparation included Modifier Flow: 1.8 mL/min Modifier Flow containing 10% Acetic Acid in Acetonitrile/Water 1:1=>resulting 0.3% Acetic Acid in Flow; and 0.5M NH₄Ac/NH₄OH-Buffer (pH 9.2) in Acetonitrile/Water 1:9=>resulting 15 mM Buffer Concentration in Flow

MS MakeUp: 0.9 mL/min 0.05% Acetic Acid in Acetonitrile/Water 1:1.

Typical Focused Gradient Timetable for e.g. 59.7% Elution Point is shown in Table 3.

TABLE 3

| Time (min) | Solv. A | Solv. B |
|---|---|---|
| −2.37 | 18.6 | 84.4 |
| 0.00 | 18.6 | 84.4 |
| 1.15 | 18.6 | 84.4 |
| 1.16 | 43.5 | 56.5 |
| 8.46 | 63.5 | 36.5 |
| 8.47 | 100 | 0 |
| 10.77 | 100 | 0 |
| 10.78 | 18.6 | 84.4 |

Typical Analytical Modifier: Column flow was 1 mL/min, Solvent A was acetonitrile, Solvent B was water, and Solvent C was 5% acetic Acid in acetonitrile/water 1:1. Typical times and relative volumes of Solvent, Solvent B, Solvent C are shown in Table 4.

TABLE 4

| Time (min) | Solv. A | Solv. B | Solv. C |
|---|---|---|---|
| 0 | 2 | 96 | 2 |
| 0.5 | 2 | 96 | 2 |
| 5.5 | 96 | 2 | 2 |
| 5.6 | 98 | 0 | 2 |
| 6.9 | 98 | 0 | 2 |
| 7.0 | 2 | 2 | 2 |

A Mass Spectrometer Detector (API-ES, positive) at UV 220 nm, 254 nm, or 310 nm was used for detection.

### Example 2

Terms and abbreviations used in the Examples are provided in Table 5.

TABLE 5
Pd dppf—[1,1′-Bis(diphenylphosphino)ferrocene]dichloropalladium(II), complex with
dichloromethane CAS 95464-05-4
DCM—dichloromethane
THF—tetrahydofuran
MeOH—methanol
celite—Diatomaceous earth, celite (R) CAS 61790-53-2
diborolane—4,4,5,5,4′,4′,5′,5′-Octamethyl-[2,2′]bi[[1,3,2]dioxaborolanyl] CAS 73183-34-3
pinacolborane—4,4,5,5-Tetramethyl-[1,3,2]dioxaborolane CAS 25015-63-8
o.n.—over night
r.t.—room temperature
eq.—equivalent
dioxane—1,4-dioxane
brine—saturated aqueous solution of NaCl
h—hour
TFA—trifluoroacetic acid
Boc—tert-Butyloxycarbonyl
catacxium—cataCXium ® A, Di(1-adamantyl)-n-butylphosphine, CAS 321921-71-5
LiHMDS—Lithium bis(trimethylsilyl)amide CAS 4039-32-1
Pd(PPh₃)₄—Palladium-tetrakis(triphenylphosphine) CAS 14221-01-3
PYBOP—(Benzotriazol-1-yloxy)tripyrrolidinophosphonium hexafluorophosphate CAS
128625-52-5
DIPEA—N,N-Diisopropylethylamine CAS 7087-68-5
DMF—N,N-Dimethylformamide CAS 68-12-2
DBAD—Di-tert-butyl azodicarboxylate CAS 870-50-8
PPh₃—Triphenylphosphine CAS 603-35-0
HOBT—1-Hydroxybenzotriazole hydrate CAS 123333-53-9
HATU—1-[Bis(dimethylamino)methylene]-1H-1,2,3-triazolo[4,5-b]pyridinium 3-oxide
hexafluorophosphate CAS 148893-10-1
EDCI—N-(3-Dimethylaminopropyl)-N′-ethylcarbodiimide hydrochloride CAS 25952-53-8
T3P—T3P ® 2,4,6-Tripropyl-1,3,5,2,4,6-trioxatriphosphorinane-2,4,6-trioxide CAS 68957-94-8
NaOtBu Sodium tert-butoxide CAS 865-48-5
X-Phos Pd G2—CAS 1310584-14-5
XPhos—CAS 564483-18-7
tBuBrettPhos Pd G3—CAS 1536473-72-9

### Example 3

Certain compounds that were used as reagents in synthesis schemes described herein were prepared by the following schemes.

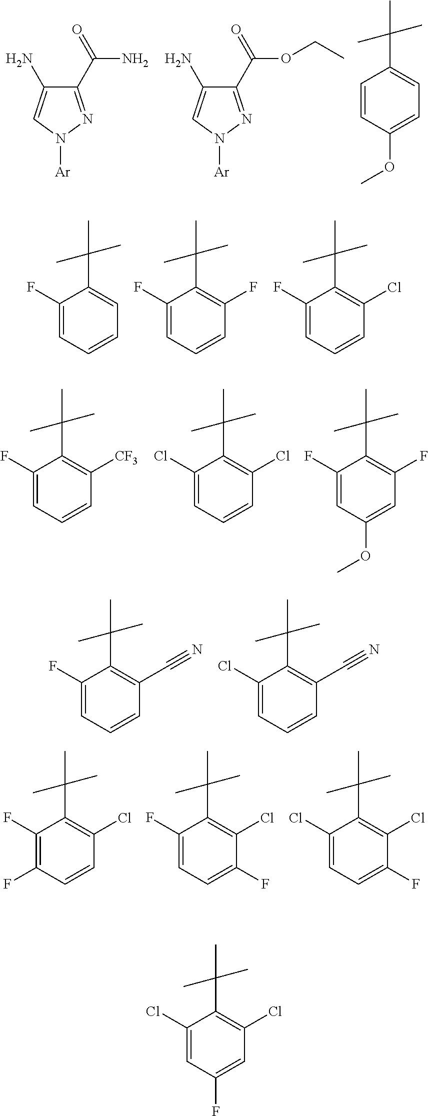

### Route 1:

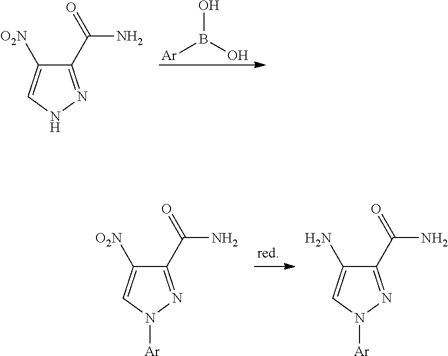

### Route 2:

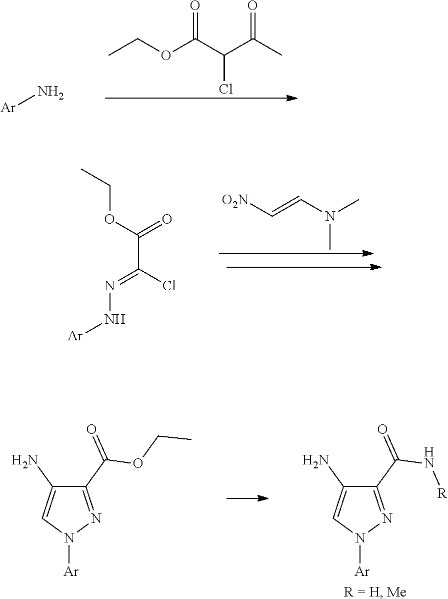

Routes 1 and 2 are known in the art and described in, for example, International Patent Publication Nos. WO2006/115845, WO2007/2248, WO2007/55941, WO2007/55942, WO2007/87129, WO2011/154327, WO2017/7756, and WO2018/64135; U.S. Patent Publication Nos. US2007/117824, US2011/306589, and US2019/192668; European Patent Publication No. EP1566384; Bioorganic and Medicinal Chemistry Letters, vol. 24, 12 (2016), p. 2794-2808; Med. Chem. Commun., vol. 4, nb. 2 (2013), p. 456-462; Journal of Medicinal Chemistry, vol. 52, nb. 23 (2009), p. 7640-7652; Zeitschrift fur Naturforschung, B, Chemical Sciences, vol. 59, nb. 10 (2004), p. 1132-1136; Tetrahedron; vol. 53, nb. 25 (1997), p. 8585-8598; Indian Journal of Chemistry—Section B Organic and Medicinal Chemistry, vol. 28, nb. 1 (1989), p. 56-60; and Gazzetta Chimica Italiana, vol. 98 (1968), p. 949, the contents of each of which are incorporated herein by reference.

### Example 4

Intermediates: Anilines.

Peptide Coupling:

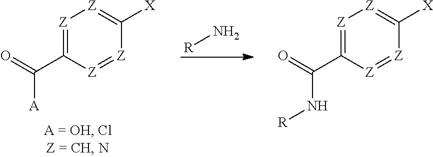

Starting from acid chloride: The amine was dissolved in DCM, cooled to 0° C., triethylamine (2 eq). and the corresponding acid chloride were added. The mixture was stirred at r.t. for 2 h. The reaction was diluted with NaHCO₃ aq., extracted with DCM, dried (Na₂SO₄), filtered and concentrated under reduced pressure.

Alternatively, standard peptide coupling protocols, e.g., using HOBT, EDCI, DIPEA or HATU, DIPEA were used.

Alternatively the following acids are used for the corresponding intermediates:

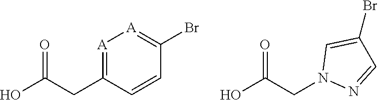

The following intermediates were synthesized using this method:

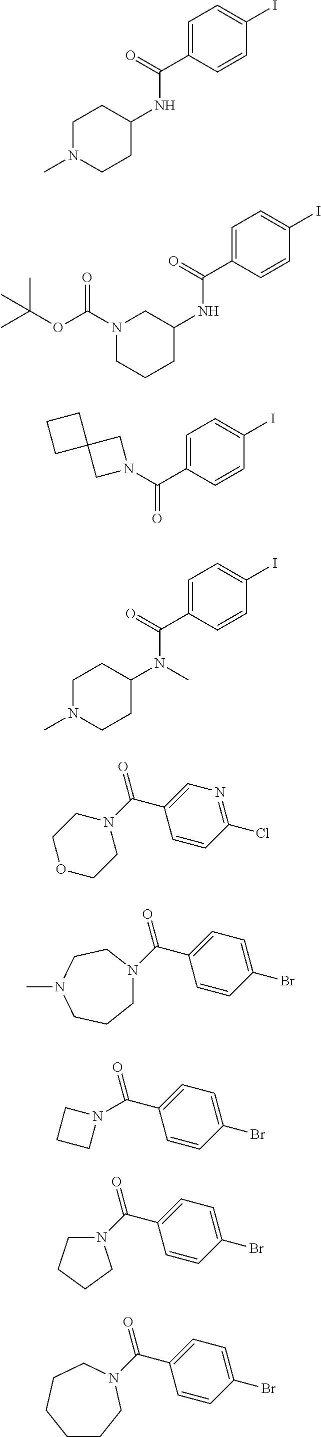

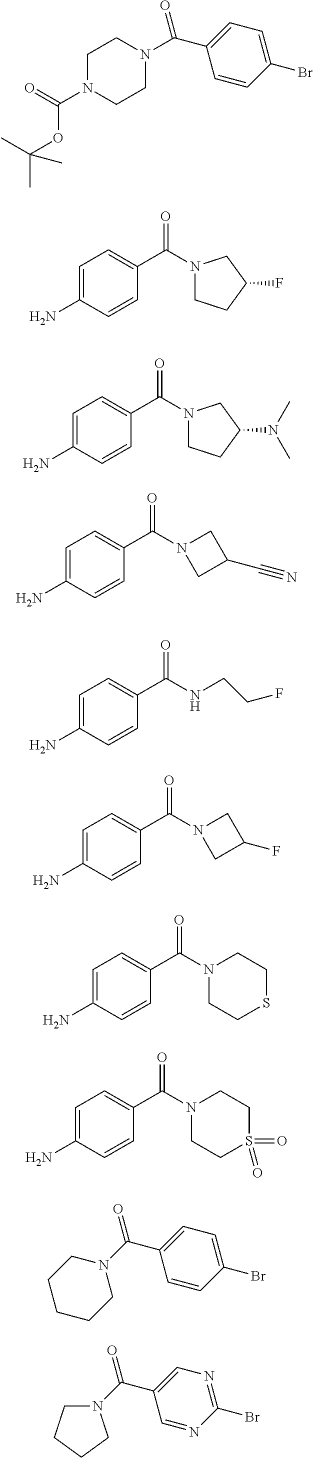

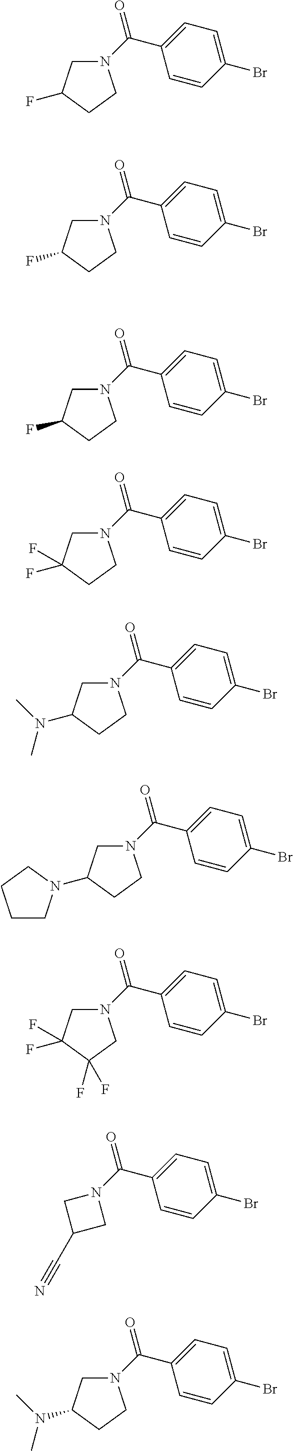

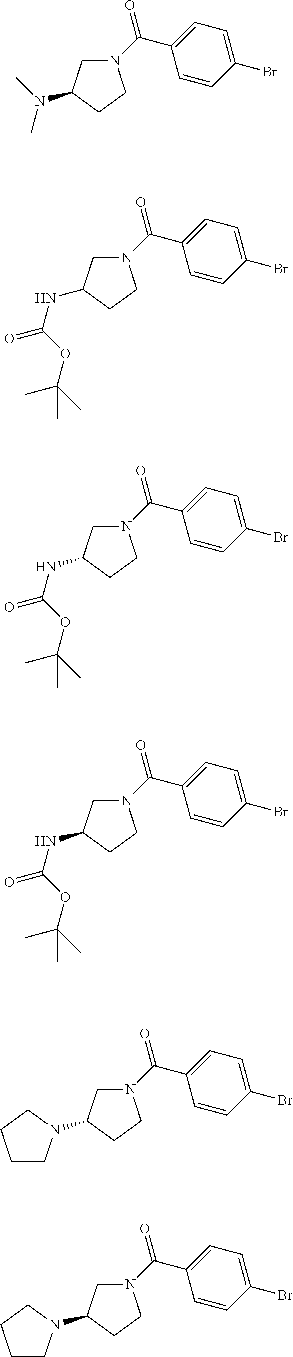 [SMILES: CN(C)[C@@H]1CCN(C(=O)c2ccc(Br)cc2)C1 ; CC(C)(C)OC(=O)NC1CCN(C(=O)c2ccc(Br)cc2)C1 ; CC(C)(C)OC(=O)N[C@H]1CCN(C(=O)c2ccc(Br)cc2)C1 ; CC(C)(C)OC(=O)N[C@@H]1CCN(C(=O)c2ccc(Br)cc2)C1 ; O=C(c1ccc(Br)cc1)N1CC[C@H](N2CCCC2)C1 ; O=C(c1ccc(Br)cc1)N1CC[C@@H](N2CCCC2)C1]

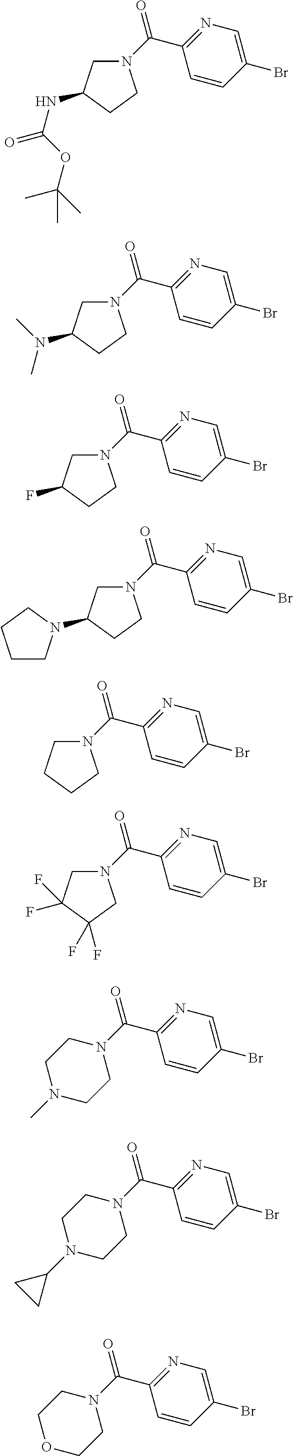

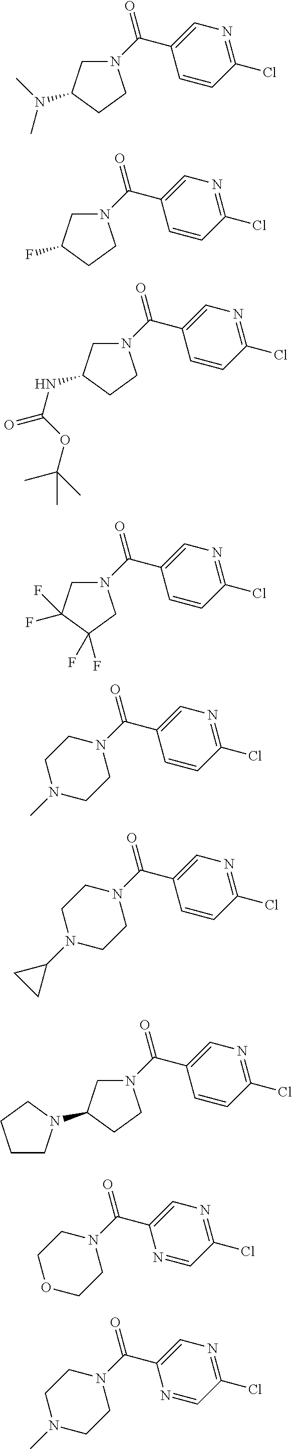

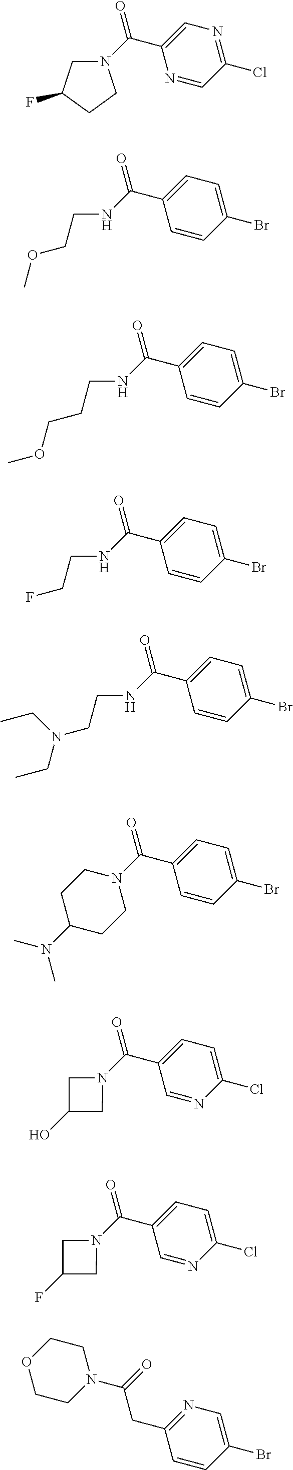

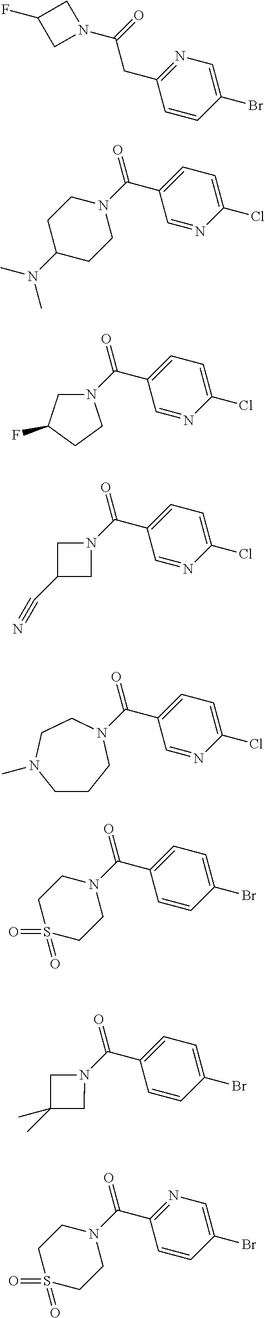

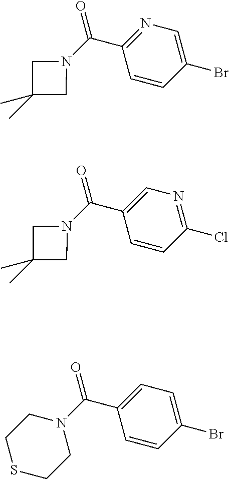

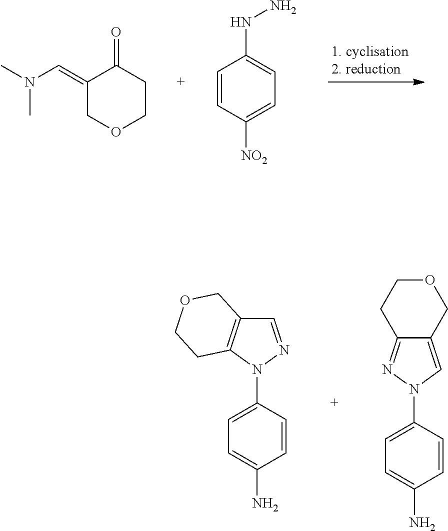

These anilines were synthesized as described in International Patent Pub. No. WO 2017/139778, the contents of which are incorporated herein by reference. The two intermediates were used as mixture and separated at the stage of the examples.

The aniline were obtained commercially or were synthesized as described above.

This method was used to synthesize Compound Nos. 101, 102, 103, 104, 105, 106, 107, 108, 109, 110, 111, 119, 123, 124, 128, 129, 145, 146, 147, 148, 162, 164, 167, 175, 176, and 177.

Compound No. 120, 2-(2,6-dichlorophenyl)-5-((4-(piperidin-4-yloxy)phenyl)amino)-2H-1,2,3-triazole-4-carboxamide, was synthesized using method A with the corresponding Boc protected reagent, followed by HCl treatment and reversed phase HPLC purification.

The following intermediate was synthesized using Route A to step 2A.

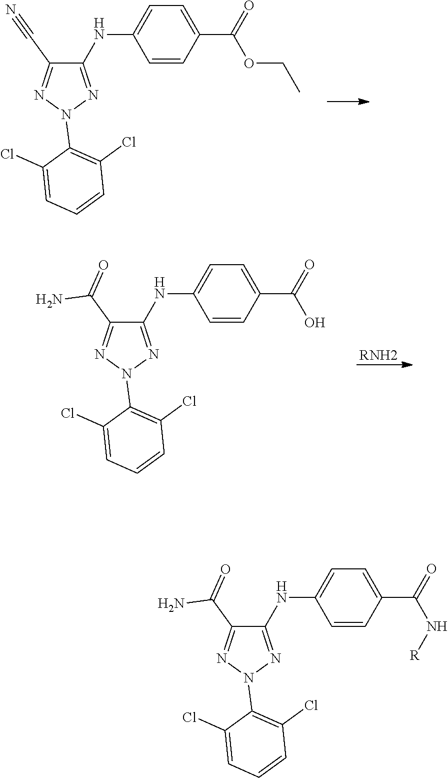

This intermediate was treated with NaOH 1M in water/THF 1/1 at 35° C. over three days. The mixture was concentrated to remove organic solvent, adjusted to pH 3-4 (HCl 1M) and extracted with EtOAc. The organic layer was dried (Na₂SO₄), filtered and concentrated under reduced pressure. The crude product was used without further purification.

The amination was performed using the conditions of aniline amide synthesis above.

This method was used to synthesize Compound Nos. 122, 163, and 166.

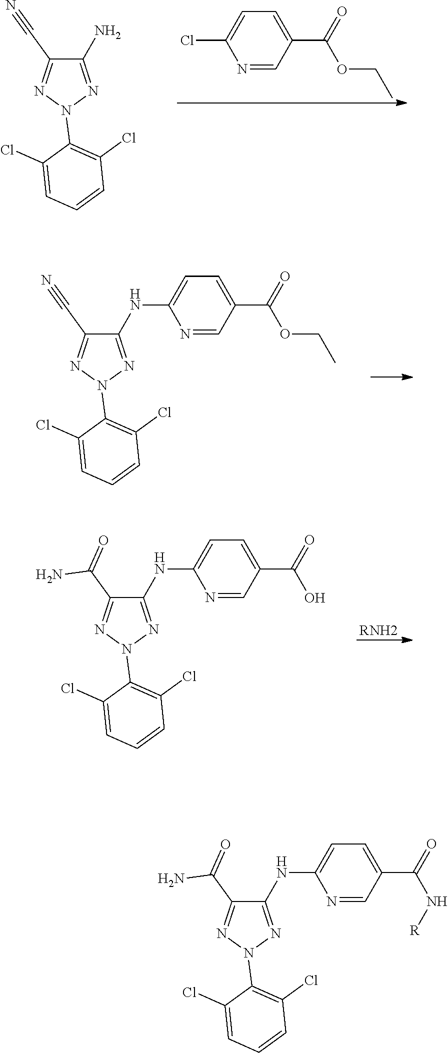

In the scheme shown above, the acid intermediate was synthesized using Route A to step 2A. This was dissolved in DMF, chloronicotinate (1 eq.) and CsCO₃ (5 eq.) was added. The mixture was heated at 60° C. o.n. The mixture was diluted with water and extracted with EtOAc. The organic layer was dried (Na₂SO₄), filtered and concentrated under reduced pressure. The crude product was purified with gradient flash chromatography.

This intermediate was treated with NaOH 1M in water/THF 1/1 at 35° C. o.n. The mixture was concentrated to remove organic solvent, adjusted to pH 3-4 (HCl 1M) and extracted with EtOAc. The organic layer was dried (Na₂SO₄), filtered and concentrated under reduced pressure. The crude product was used without further purification.

The amination was performed using the conditions of aniline amide synthesis above.

This method was used to synthesize Compound Nos. 133, 149, 150, 153, 154, and 165.

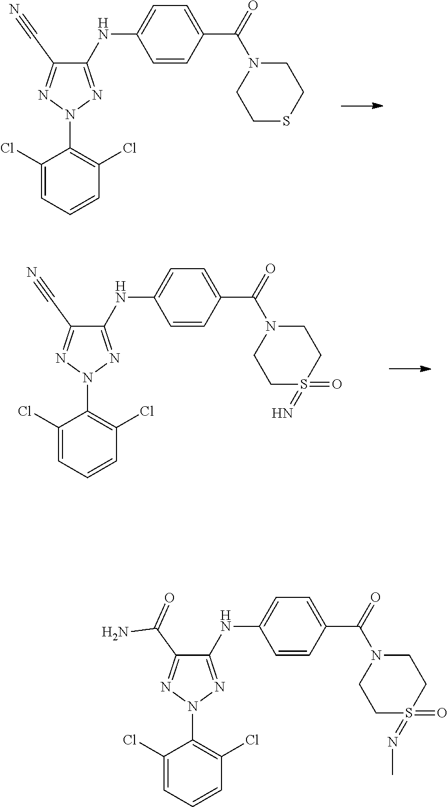 [SMILES: N#Cc1nn(-c2c(Cl)cccc2Cl)nc1Nc1ccc(C(=O)N2CCSCC2)cc1 ; N#Cc1nn(-c2c(Cl)cccc2Cl)nc1Nc1ccc(C(=O)N2CCS(=N)(=O)CC2)cc1 ; CN=S1(=O)CCN(C(=O)c2ccc(Nc3nn(-c4c(Cl)cccc4Cl)nc3C(N)=O)cc2)CC1]

In the scheme shown above, the thiomorpholine intermediate was synthesized following Route A to step 2A. This intermediate was dissolved in MeOH, ammonium acetate (2 eq.) and iodobenzene diacetate (3 eq.) were added. The mixture was stirred for 2.5 h at r.t., then concentrated, diluted with water and extracted with DCM. The organic layer was dried (Na₂SO₄), filtered and concentrated under reduced pressure. The crude product was purified by gradient flash chromatography.

The product from the step before was dissolved in MeOH. Formaldehyde (10 eq., 37% in water) was added, the pH was adjusted with NaOH 1M and HOAc against bromocresole green, followed by sodium cyanoborohydride (2.5 eq.). The mixture was stirred for 2 h, the adjustment and the addition of cyano borohydride was repeated. The mixture was cooled to 0° C., water and NaHCO₃ aq. sat was added an the mixture extracted with DCM. The organic layer was dried (Na₂SO₄), filtered and concentrated under reduced pressure. The crude product was used without further purification.

The hydrolysis to the amide was following step 3A.

Compound No. 172, 2-(2,6-dichlorophenyl)-5-((1-(methylsulfonyl)-1H-pyrazol-4-yl)amino)-2H-1,2,3-triazole-4-carboxamide, was synthesized using this procedure.

Route B was used to synthesize Compound Nos. 121, 125, 126, 131, and 132.

The halogenids were obtained commercially or synthesized as described above:

Route C, using the condition A2.2 from pyrazole section, was used to synthesize Compound Nos. 127, 130, 136, 137, and 140.

The halogenids were obtained commercially or synthesized as described above.

Route C, using the condition A2.3 from pyrazole section, was used to synthesize Compound Nos. 112, 113, 114, 115, 141, 142, 143, 144, 152, 155, 156, 157, 158, 159, 160, 168, 172, and 173.

The halogenids were obtained commercially or synthesized as described above.

Route D was used to synthesize Compound Nos. 151, 169, and 170.

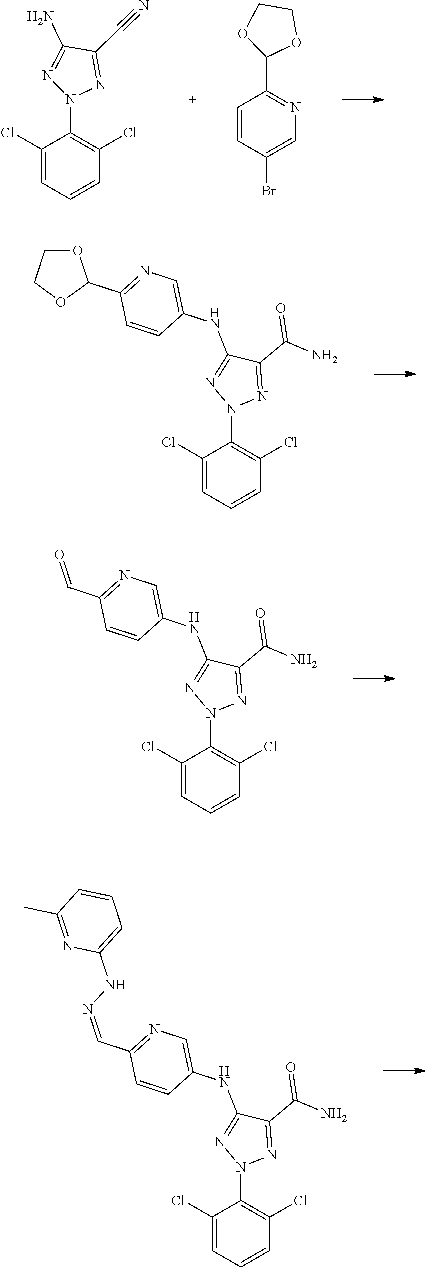

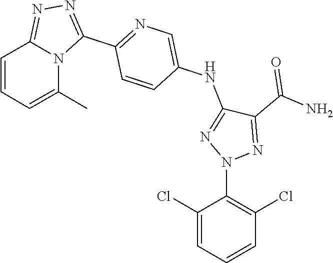 [SMILES: Cc1cccc2nnc(-c3ccc(Nc4nn(-c5c(Cl)cccc5Cl)nc4C(N)=O)cn3)n12]

In the scheme shown above, the intermediate form step 2C was reacted under the conditions of A2.3 (pyrazole section), followed by treatment with 2 m HCl aq. in THF for 2. The mixture was concentrated, diluted with water and extracted with EtOAc. The organic layer was dried (Na₂SO₄), filtered and concentrated under reduced pressure.

The aldehyde building block was dissolved in EtOH. The hydrazine derivative was added and the mixture refluxed for 1 h. The mixture was concentrated under reduced pressure. The crude product was used without further purification.

This intermediate was dissolved in DCM. Iodobenzene diacetate (10 eq.) was added and the mixture was stirred for 3 h at r.t. The mixture was filtered through celite, concentrated and purified by reversed phase HPLC.

This method, with appropriate hydrazine derivatives, was used to synthesize Compound Nos. 139 and 161.

Other compounds that may be made using the schemes described above are shown below.

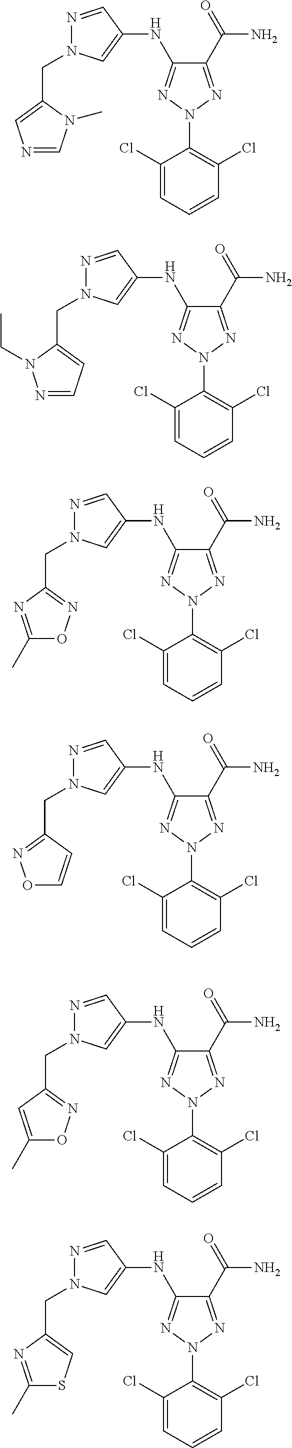

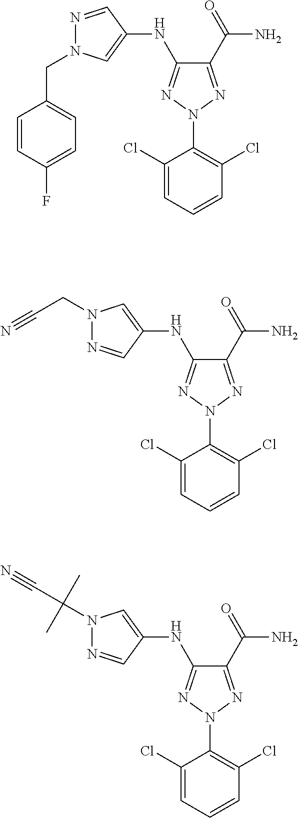 [SMILES: NC(=O)c1nn(-c2c(Cl)cccc2Cl)nc1Nc1cnn(Cc2ccc(F)cc2)c1 ; N#CCn1cc(Nc2nn(-c3c(Cl)cccc3Cl)nc2C(N)=O)cn1 ; CC(C)(C#N)n1cc(Nc2nn(-c3c(Cl)cccc3Cl)nc2C(N)=O)cn1]

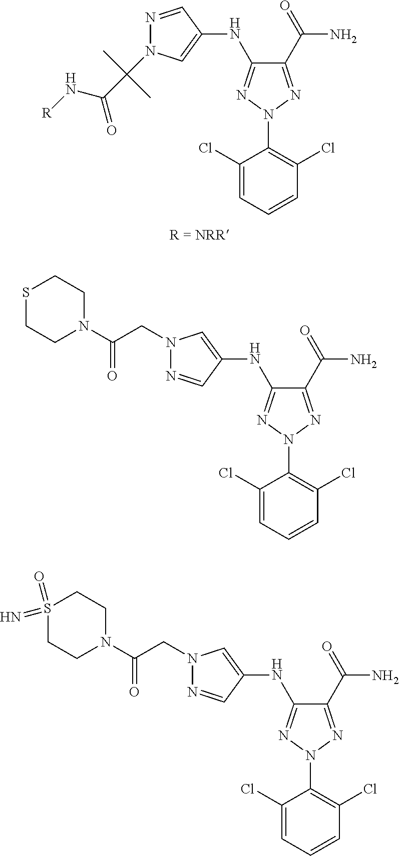

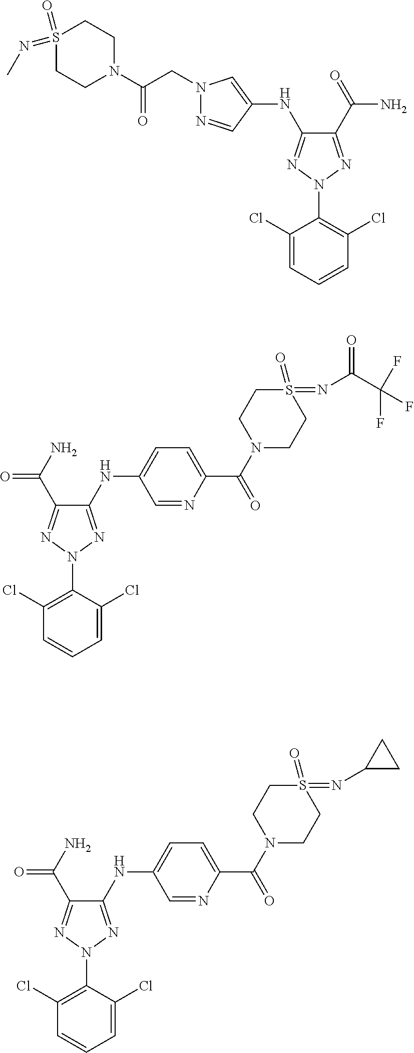 [SMILES: CN=S1(=O)CCN(C(=O)Cn2cc(Nc3nn(-c4c(Cl)cccc4Cl)nc3C(N)=O)cn2)CC1 ; NC(=O)c1nn(-c2c(Cl)cccc2Cl)nc1Nc1ccc(C(=O)N2CCS(=O)(=NC(=O)C(F)(F)F)CC2)nc1 ; NC(=O)c1nn(-c2c(Cl)cccc2Cl)nc1Nc1ccc(C(=O)N2CCS(=O)(=NC3CC3)CC2)nc1]

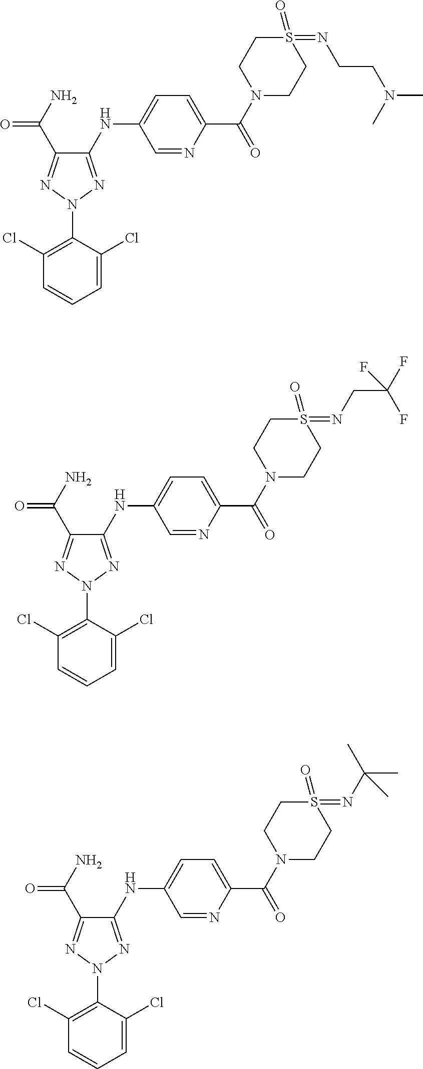 [SMILES: CN(C)CCN=S1(=O)CCN(C(=O)c2ccc(Nc3nn(-c4c(Cl)cccc4Cl)nc3C(N)=O)cn2)CC1 ; NC(=O)c1nn(-c2c(Cl)cccc2Cl)nc1Nc1ccc(C(=O)N2CCS(=O)(=NCC(F)(F)F)CC2)nc1 ; CC(C)(C)N=S1(=O)CCN(C(=O)c2ccc(Nc3nn(-c4c(Cl)cccc4Cl)nc3C(N)=O)cn2)CC1]

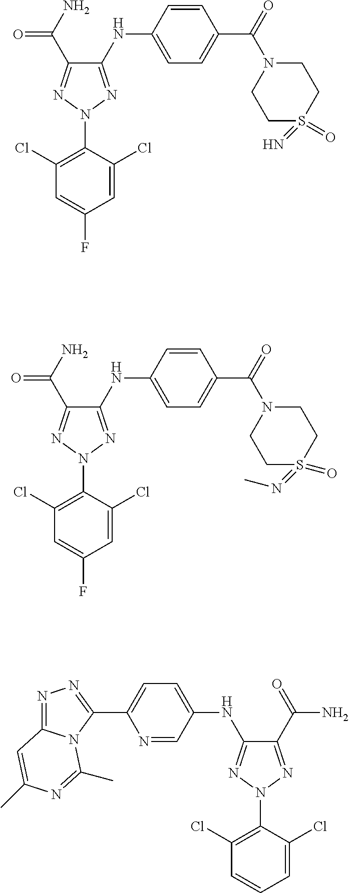 [SMILES: N=S1(=O)CCN(C(=O)c2ccc(Nc3nn(-c4c(Cl)cc(F)cc4Cl)nc3C(N)=O)cc2)CC1 ; CN=S1(=O)CCN(C(=O)c2ccc(Nc3nn(-c4c(Cl)cc(F)cc4Cl)nc3C(N)=O)cc2)CC1 ; Cc1cc2nnc(-c3ccc(Nc4nn(-c5c(Cl)cccc5Cl)nc4C(N)=O)cn3)n2c(C)n1]

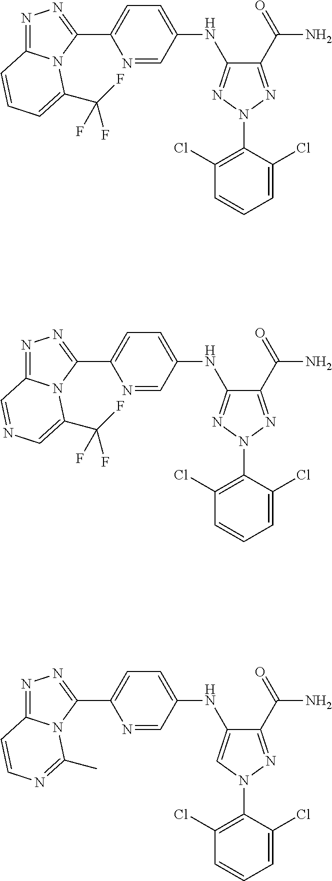 [SMILES: NC(=O)c1nn(-c2c(Cl)cccc2Cl)nc1Nc1ccc(-c2nnc3cccc(C(F)(F)F)n23)nc1 ; NC(=O)c1nn(-c2c(Cl)cccc2Cl)nc1Nc1ccc(-c2nnc3cncc(C(F)(F)F)n23)nc1 ; Cc1nccc2nnc(-c3ccc(Nc4cn(-c5c(Cl)cccc5Cl)nc4C(N)=O)cn3)n12]

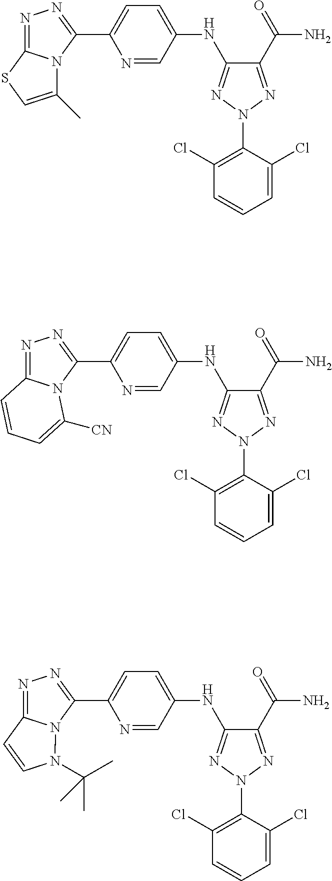 [SMILES: Cc1csc2nnc(-c3ccc(Nc4nn(-c5c(Cl)cccc5Cl)nc4C(N)=O)cn3)n12 ; N#Cc1cccc2nnc(-c3ccc(Nc4nn(-c5c(Cl)cccc5Cl)nc4C(N)=O)cn3)n12 ; CC(C)(C)n1ccc2nnc(-c3ccc(Nc4nn(-c5c(Cl)cccc5Cl)nc4C(N)=O)cn3)n21]

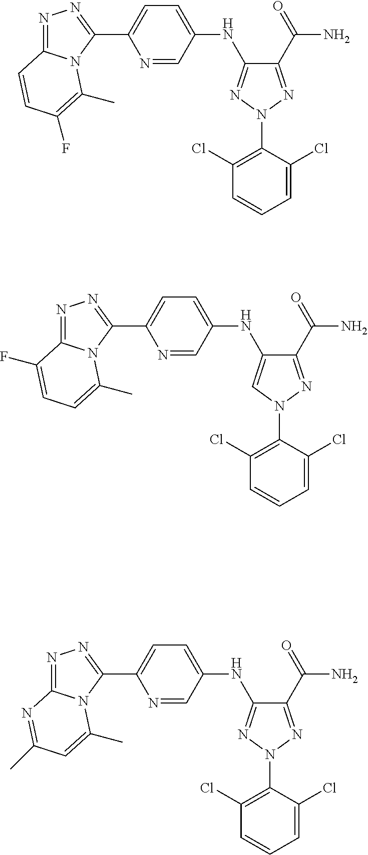 [SMILES: Cc1c(F)ccc2nnc(-c3ccc(Nc4nn(-c5c(Cl)cccc5Cl)nc4C(N)=O)cn3)n12 ; Cc1ccc(F)c2nnc(-c3ccc(Nc4cn(-c5c(Cl)cccc5Cl)nc4C(N)=O)cn3)n12 ; Cc1cc(C)n2c(-c3ccc(Nc4nn(-c5c(Cl)cccc5Cl)nc4C(N)=O)cn3)nnc2n1]

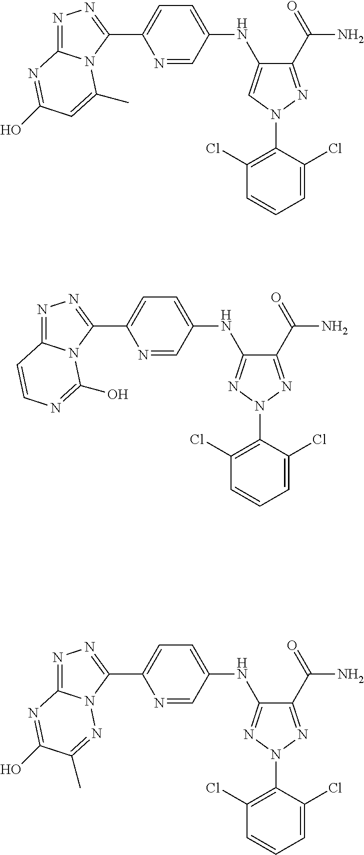 [SMILES: Cc1cc(O)nc2nnc(-c3ccc(Nc4cn(-c5c(Cl)cccc5Cl)nc4C(N)=O)cn3)n12 ; NC(=O)c1nn(-c2c(Cl)cccc2Cl)nc1Nc1ccc(-c2nnc3ccnc(O)n23)nc1 ; Cc1nn2c(-c3ccc(Nc4nn(-c5c(Cl)cccc5Cl)nc4C(N)=O)cn3)nnc2nc1O]

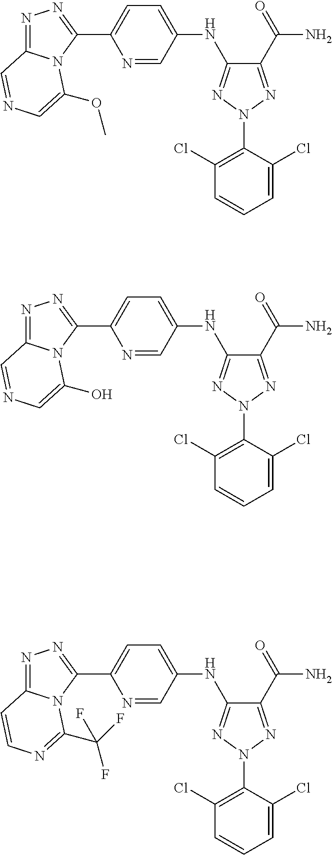 [SMILES: COc1cncc2nnc(-c3ccc(Nc4nn(-c5c(Cl)cccc5Cl)nc4C(N)=O)cn3)n12 ; NC(=O)c1nn(-c2c(Cl)cccc2Cl)nc1Nc1ccc(-c2nnc3cncc(O)n23)nc1 ; NC(=O)c1nn(-c2c(Cl)cccc2Cl)nc1Nc1ccc(-c2nnc3ccnc(C(F)(F)F)n23)nc1]

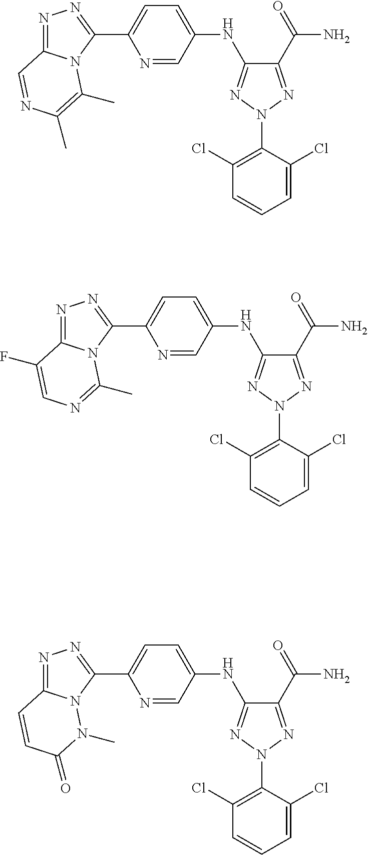 [SMILES: Cc1ncc2nnc(-c3ccc(Nc4nn(-c5c(Cl)cccc5Cl)nc4C(N)=O)cn3)n2c1C ; Cc1ncc(F)c2nnc(-c3ccc(Nc4nn(-c5c(Cl)cccc5Cl)nc4C(N)=O)cn3)n12 ; Cn1c(=O)ccc2nnc(-c3ccc(Nc4nn(-c5c(Cl)cccc5Cl)nc4C(N)=O)cn3)n21]

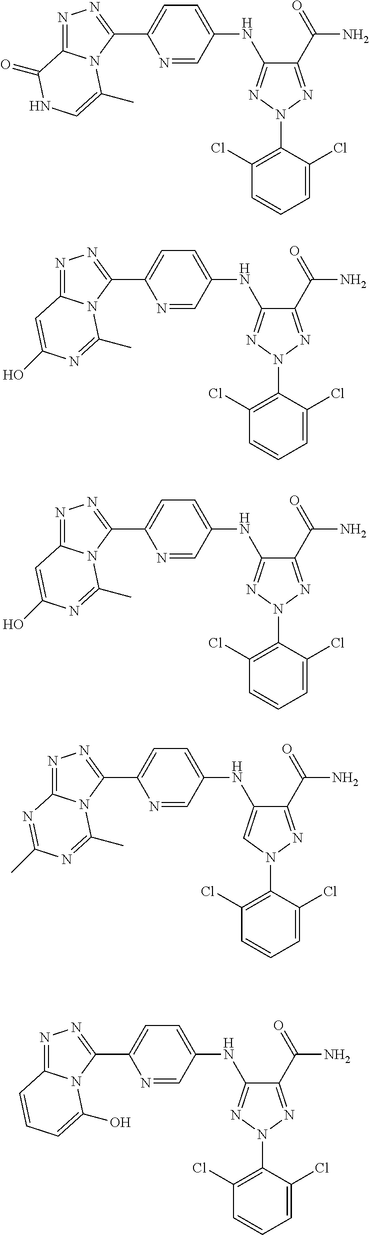

### Example 5

Piperazine Modifications:

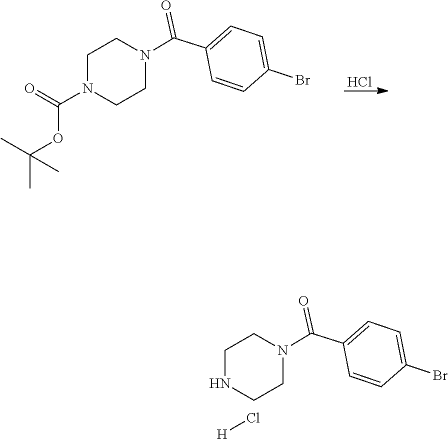

The Boc protected piperazine was dissolved in dry MeOH followed by the addition of 4M HCl in dioxane (40% v/v). The mixture was stirred o.n. The product was collected by filtration and used without further purification.

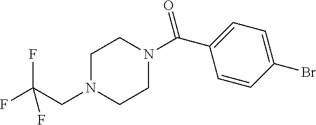 [SMILES: O=C(c1ccc(Br)cc1)N1CCN(CC(F)(F)F)CC1]

The HCl salt from above was suspended in THF (dry), followed by the addition of 2,2,2-trifluoroethyl trifluoromethanesulfonate (3 eq.) and DIPEA (4 eq.). The mixture was stirred at 65° C. o.n. The mixture was diluted with ethyl acetate and washed with NaHCO₃ aq. The organic layer was dried (Na₂SO₄) filtered and concentrated under reduced pressure. The crude product was purified by gradient flash chromatography.

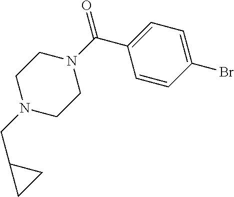 [SMILES: O=C(c1ccc(Br)cc1)N1CCN(CC2CC2)CC1]

The HCl salt from above and cyclopropyl aldehyde (1.2 eq.) were dissolved in DCE DCE or DCM? If DCE need to define in the table of abbreviations vide supra. After 10 min sodium triacetoxyborohydride (1.5. eq.) was added. The mixture was stirred at r.t. o.n. The mixture was diluted with ethyl acetate and washed with NaHCO₃ aq. The organic layer was dried (Na₂SO₄) filtered and concentrated under reduced pressure. The crude product was purified by gradient flash chromatography.

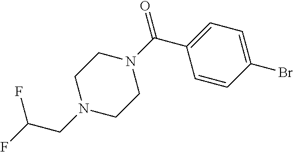 [SMILES: O=C(c1ccc(Br)cc1)N1CCN(CC(F)F)CC1]

The HCl salt from above was suspended in THF (dry), followed by the addition of 2,2-difluoroethyl trifluoromethanesulfonate (3 eq.) and DIPEA (4 eq.). The mixture was stirred at 65° C. o.n. The mixture was diluted with ethyl acetate and washed with NaHCO₃ aq. The organic layer was dried (Na₂SO₄) filtered and concentrated under reduced pressure. The crude product was purified by gradient flash chromatography.

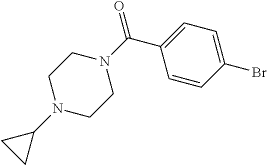 [SMILES: O=C(c1ccc(Br)cc1)N1CCN(C2CC2)CC1]

The HCl salt from above and (1-Ethoxycyclopropoxy)trimethylsilane (10 eq.) were dissolved in MeOH. Acetic acid (16 eq.) was added dropwise, followed by sodium cyanoborohydride (12. eq.).). The mixture was stirred at r.t. o.n. The mixture was diluted with ethyl acetate and washed with NaHCO₃ aq. The organic layer was dried (Na₂SO₄) filtered and concentrated under reduced pressure. The crude product was purified by gradient flash chromatography.

Special Modifications

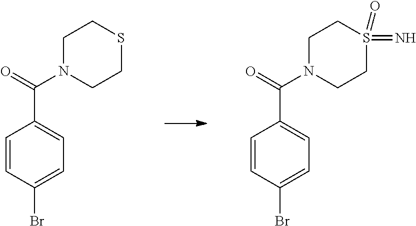 [SMILES: O=C(c1ccc(Br)cc1)N1CCSCC1 ; N=S1(=O)CCN(C(=O)c2ccc(Br)cc2)CC1]

The thiomorpholine amide was dissolved in MeOH, followed by the addition of ammonium acetate (2 eq.) and iodobenzene diacetate (2.5 eq.). The mixture was stirred for 30 min at r.t. The mixture was concentrated, diluted with water and extracted with DCM. The organic layer was dried (Na₂SO₄), filtered and concentrated under reduced pressure. The crude product was purified by gradient flash chromatography.

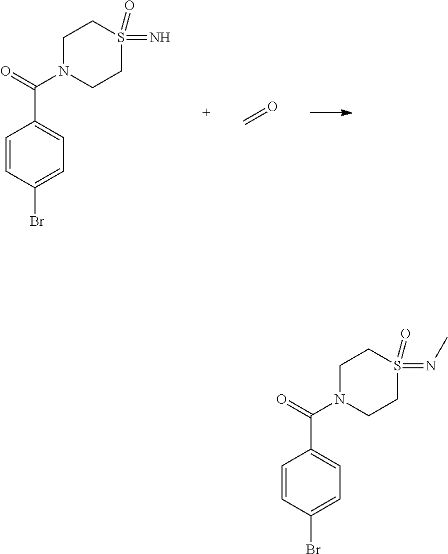

The imino-oxidothiomorpholino derivative was dissolved in MeOH, formaldehyde (10 eq., 37% in water) and sodium cyano borohydride (2.5 eq.) was added. The mixture was basified with NaOH 1M until the color change of bromcresol green. After 20 min additional NaBH₃CN (2.5 eq.). The pH was adjusted with HOAc. This was repeated three times. The mixture was basified with NaOH 1M and NaHCO₃ aq. sat. The mixture was extracted with DCM, the organic layer dried (Na₂SO₄), filtered and concentrated under reduced pressure. The crude product was purified by gradient flash chromatography.

### Example 6

Suzuki Type Coupling:

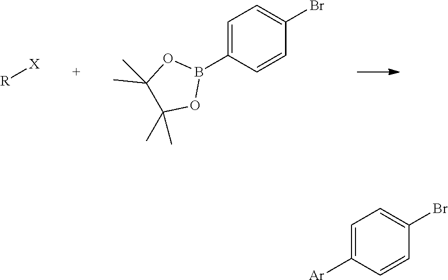

General procedure: Bromophenyl boronic ester, haloarene (2 eq.), K₂CO₃ (3 eq.) were dissolved in dioxane water (5/1). The mixture is degassed with N₂, then Pd(PPh₃)₄ (0.1 eq.) was added. The mixture was heated at 100° C. o.n. The solvent was evaporated under reduced pressure. The crude product was purified by gradient flash chromatography.

The following intermediates were synthesized using this method:

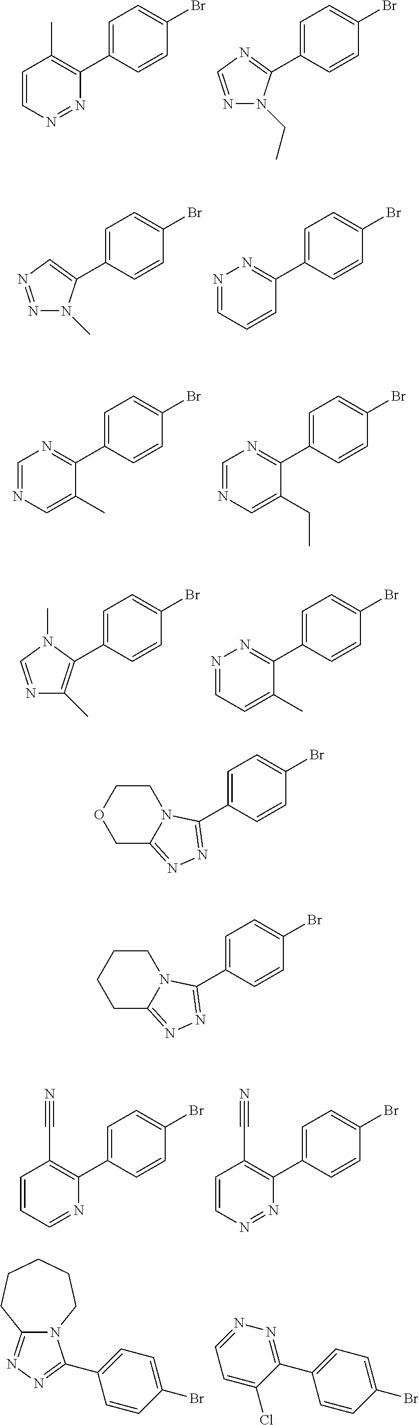

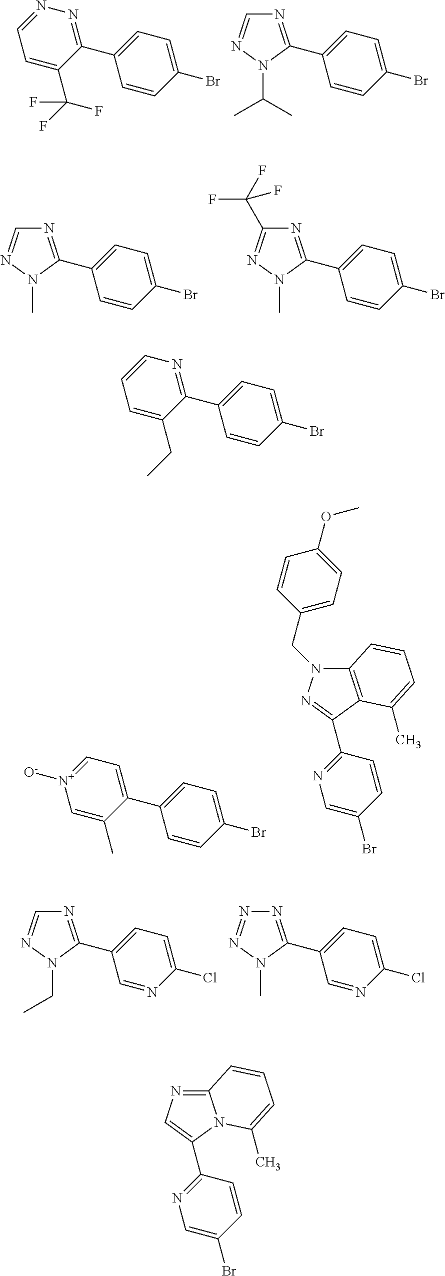

### Example 7

Ullmann Type Coupling

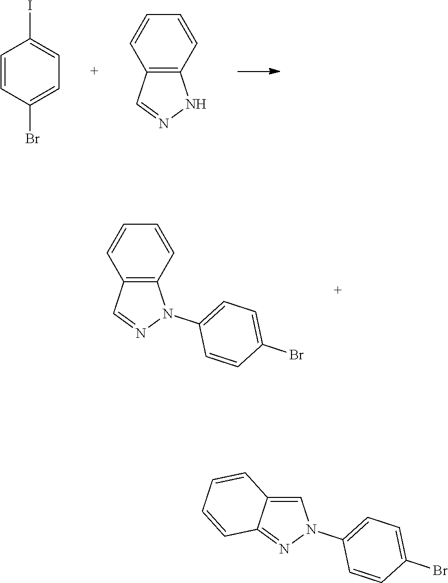 [SMILES: Brc1ccc(I)cc1 ; c1ccc2[nH]ncc2c1 ; Brc1ccc(-n2ncc3ccccc32)cc1 ; "+" ; Brc1ccc(-n2cc3ccccc3n2)cc1]

General procedure: Bromoiodobenzene, pyrazolo compound (1 eq.); K₂CO₃ (2.1 eq.) dimethyl glycine and CuI were dissolved in DMSO. During the degassing the reaction turned blue. The reaction was stirred at 100° C. for 1 h. The mixture was diluted with ethyl acetate and water. The aq. layer was extracted with ethyl acetate several times. The combined organic layers were washed with NH₄Cl aq, and brine. The organic layer was dried (Na₂SO₄), filtered and concentrated under reduced pressure. The resulting product was used without further purification as a mixture of isomers.

The following intermediates were synthesized using this procedure:

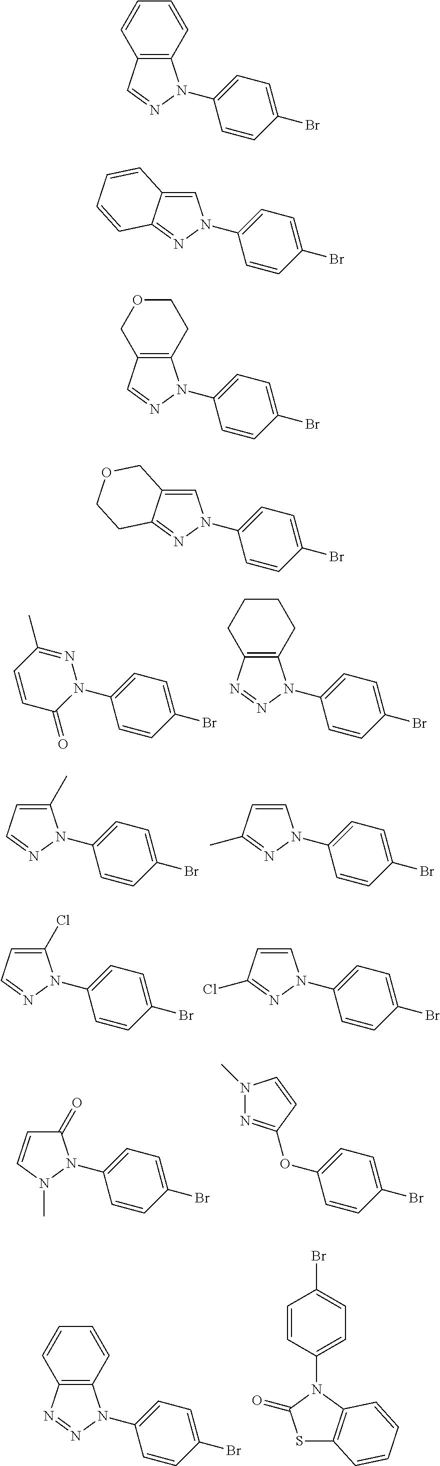

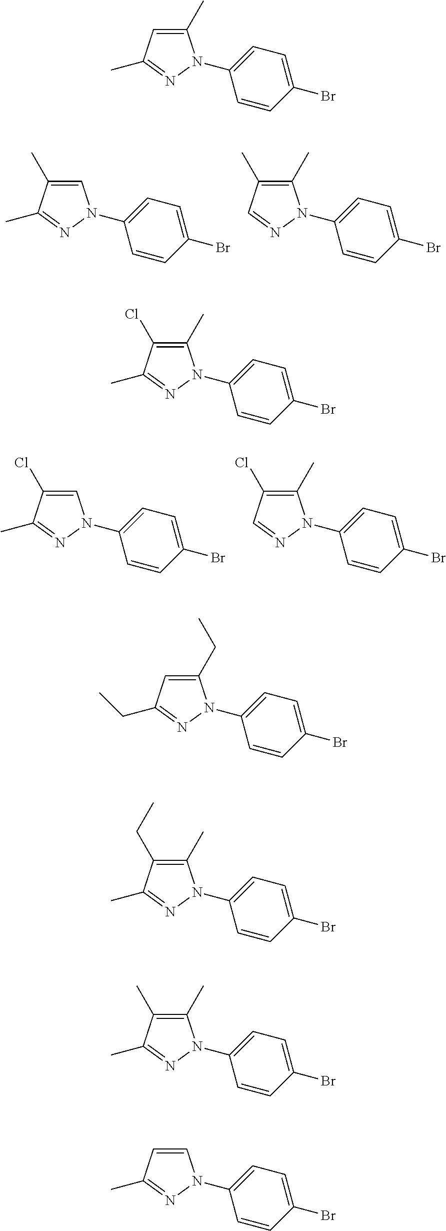

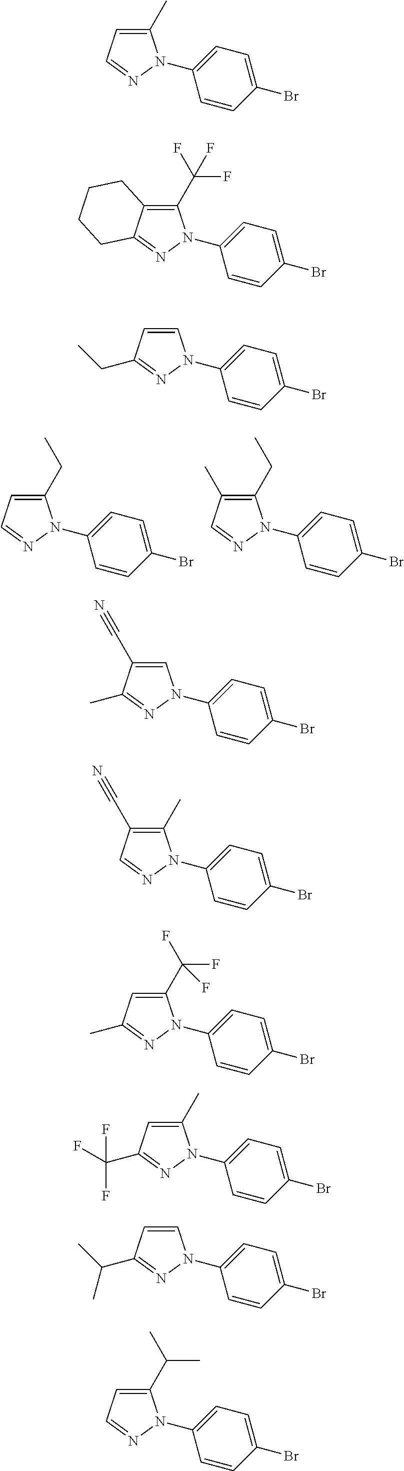

3-Aryl,4-Alky 1,2,4 Triazole Formation.

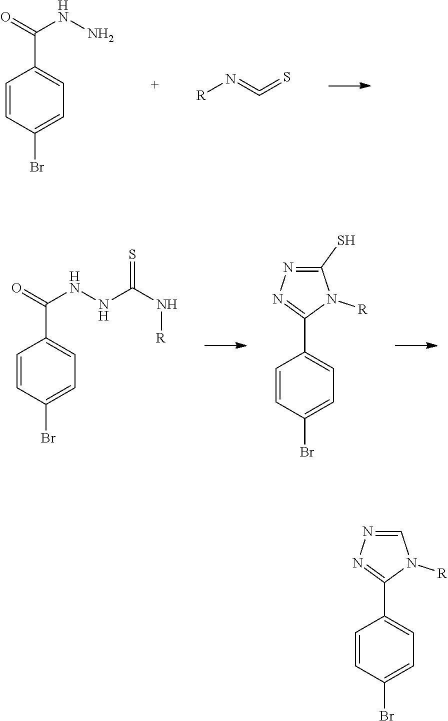

The following intermediates were synthesized in procedures known in the art and described in, for example, Journal of Medicinal Chemistry, 2018, vol. 61, no. 18, p. 8226-8240, the contents of which are incorporated herein by reference.

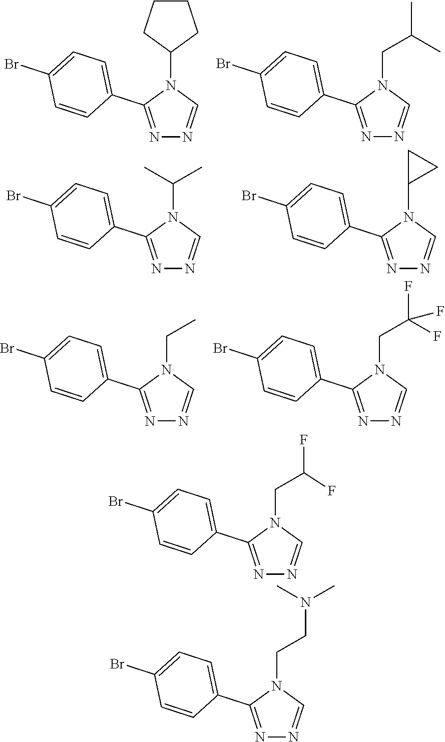

### Special Examples

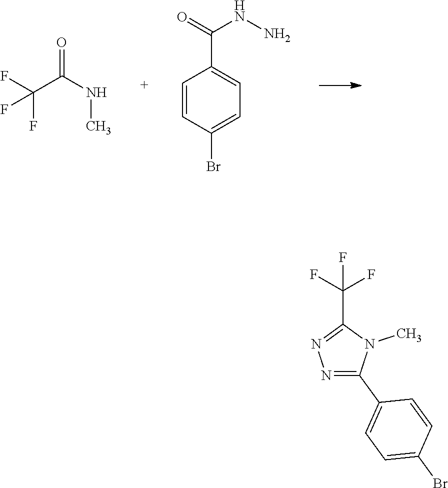

The intermediate shown above was synthesized as described in Org. Lett. 2015, 17, 1184-1187, the contents of which are incorporated herein by reference.

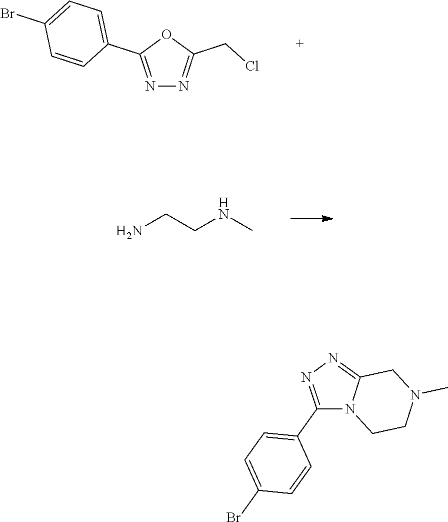

The intermediate shown above was synthesized as described in Org. Lett. 2005, 7, 6, 1039-1042, the contents of which are incorporated herein by reference.

1-Alky-2-Arylimidazole

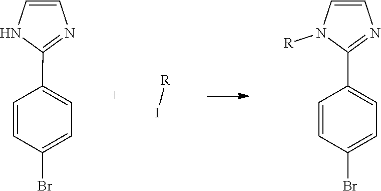

The following compounds were synthesized as described in Org. Lett. 2017, 19, 6, 1450-1453, the contents of which are incorporated herein by reference.

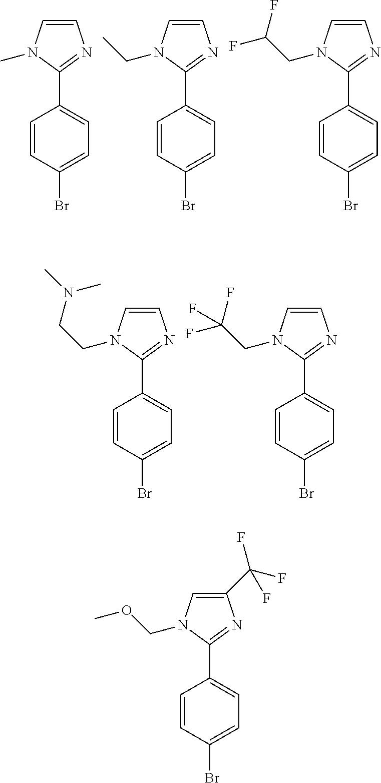

1-Aryl,5-Alkyl Tetrazoles

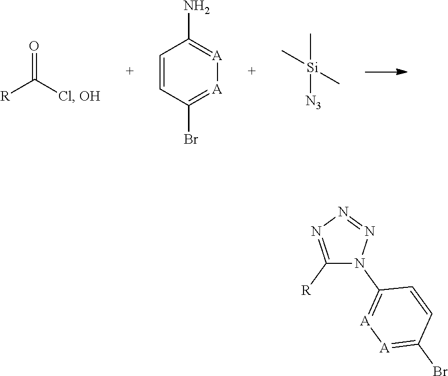

The following compounds were synthesized as described in EUR JOC 2016, 2383-2387, the contents of which are incorporated herein.

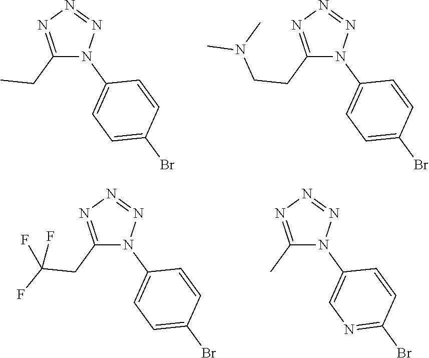 [SMILES: CCc1nnnn1-c1ccc(Br)cc1 ; CN(C)CCc1nnnn1-c1ccc(Br)cc1 ; FC(F)(F)Cc1nnnn1-c1ccc(Br)cc1 ; Cc1nnnn1-c1ccc(Br)nc1]

3-Trifluoromethyl, 5-Alkyl, 1H-Triazole

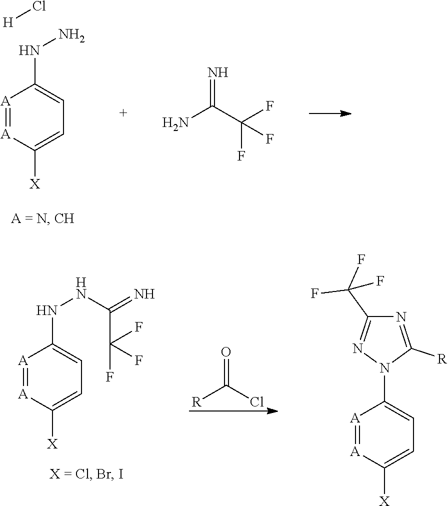

The following compounds were synthesized as described in, e.g., US2004/106612 A1, WO2018/48969 A1, US2005/75507 A1, the contents of which are incorporated herein by reference.

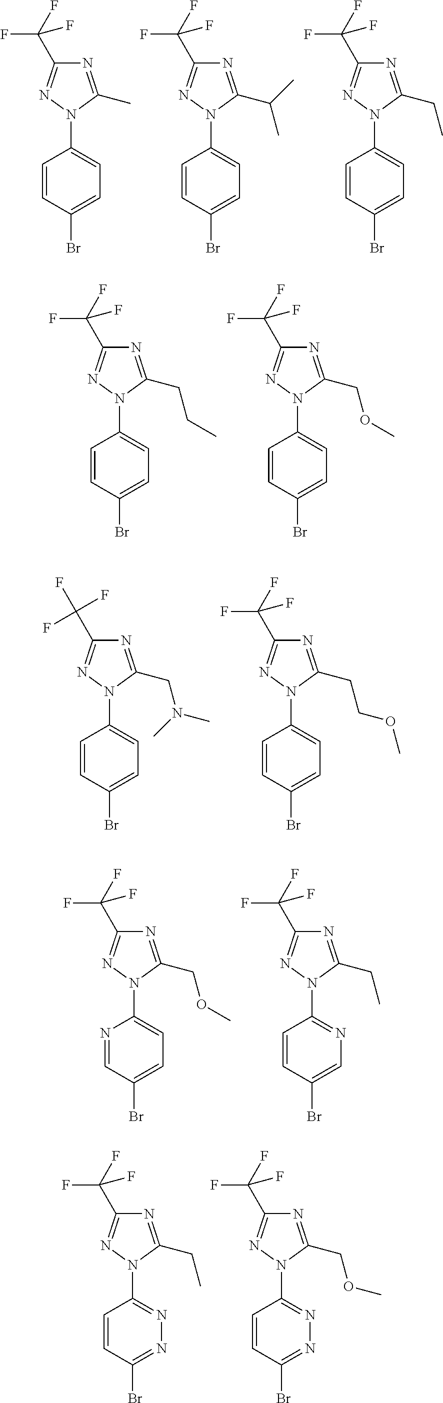

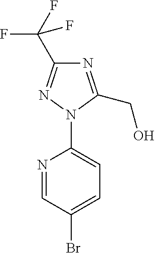 [SMILES: OCc1nc(C(F)(F)F)nn1-c1ccc(Br)cn1]

### 5-Aryl-1-alkyl-3-(trifluoromethyl)-1H-1,2,4-triazoles

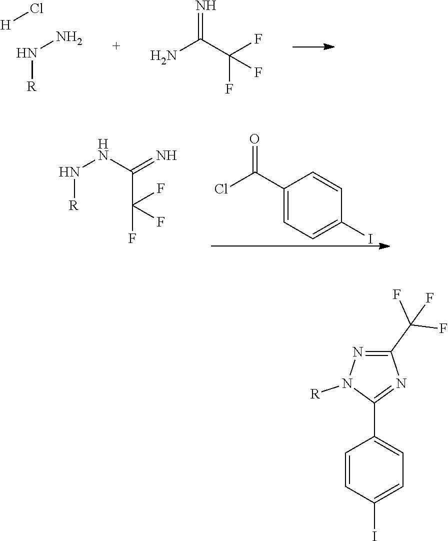

The corresponding hydrazine hydrochloride was dissolved in THF/MeOH, followed by triethylamine (1.1 eq.). After 30 min trifluoro amidine (1.1. eq.) The mixture was stirred for 6 h, diluted with water and extracted with EtOAc. The organic layer was dried (Na₂SO₄), filtered and concentrated under reduced pressure. The crude product was used without further purification.

The product from step1 was dissolved in dioxane, pyridine (1 eq.) was added and the was degassed with N₂. After 10 min the acid chloride was added. The mixture was stirred at 105° C. o.n. The mixture was diluted with water and extracted with EtOAc. The organic layer was dried (Na₂SO₄), filtered and concentrated under reduced pressure. The crude product was purified by gradient flash chromatography.

The following intermediates were synthesized according to this procedure:

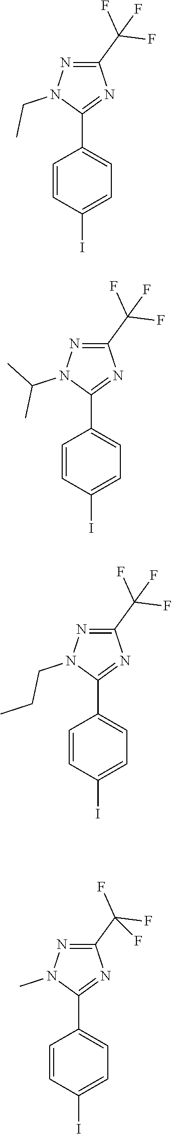 [SMILES: CCn1nc(C(F)(F)F)nc1-c1ccc(I)cc1 ; CC(C)n1nc(C(F)(F)F)nc1-c1ccc(I)cc1 ; CCCn1nc(C(F)(F)F)nc1-c1ccc(I)cc1 ; Cn1nc(C(F)(F)F)nc1-c1ccc(I)cc1]

1-Alkyl,2-Aryl,4-Trifluoromethyl Imidazole

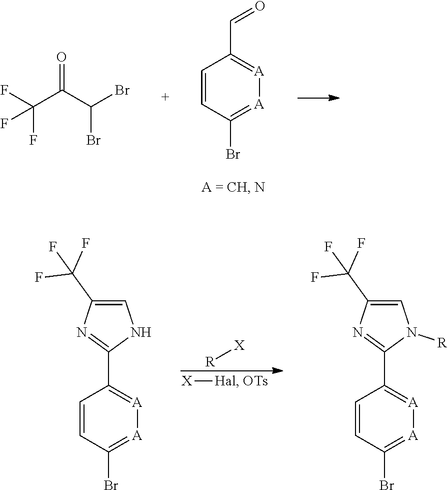

The syntheses were performed as described in US20200079776A1, the contents of which are incorporated herein by reference.

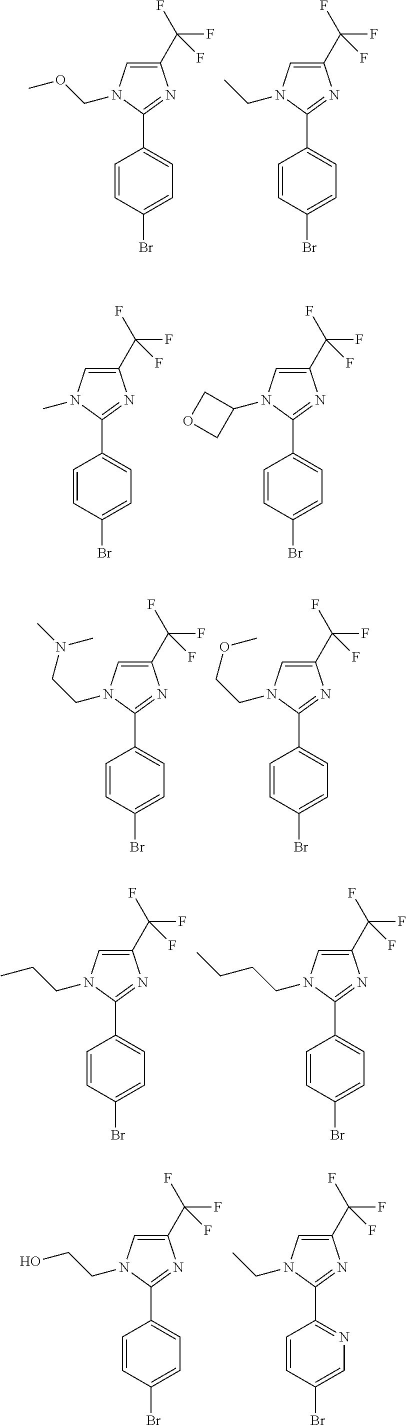

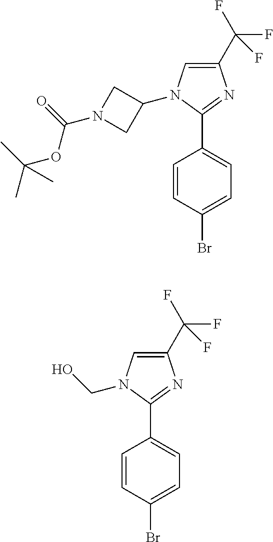 [SMILES: CC(C)(C)OC(=O)N1CC(n2cc(C(F)(F)F)nc2-c2ccc(Br)cc2)C1 ; OCn1cc(C(F)(F)F)nc1-c1ccc(Br)cc1]

Later Modifications:

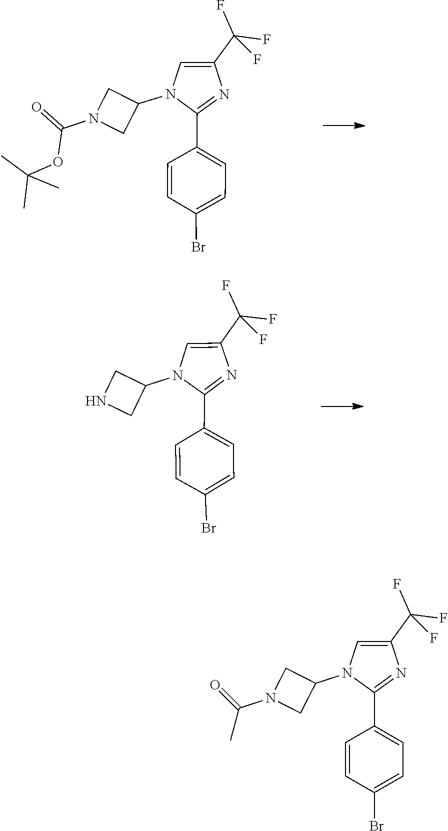 [SMILES: CC(C)(C)OC(=O)N1CC(n2cc(C(F)(F)F)nc2-c2ccc(Br)cc2)C1 ; FC(F)(F)c1cn(C2CNC2)c(-c2ccc(Br)cc2)n1 ; CC(=O)N1CC(n2cc(C(F)(F)F)nc2-c2ccc(Br)cc2)C1]

The protected compound was dissolved in DCM/TFA (2/1) and stirred for 5 h. The reaction was quenched by adding NaHCO₃ aq. and extracted with DCM/MeOH (9/1). The organic layer was washed with brine, dried, filtered and concentrated under reduced pressure. The product was used without further purification.

The product from the first step was dissolved in THF, AcCl (1.5 eq.) and Net3 (3 eq.) were added and the mixture stirred for 10 min. The mixture was concentrated, partitioned between water and DCM, the organic layer dried (Na₂SO₄), filtered and concentrated under reduced pressure. The product was used without further purification.

1-Alkyl,2-Aryl,4-Cyano Imidazole

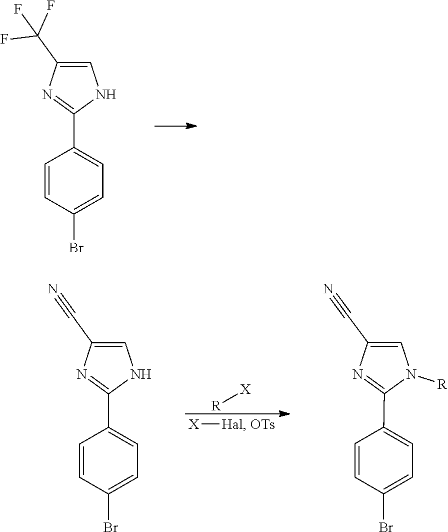

The trifluoro aryl imidazole was dissolved in MeOH, ammonia (20 mL/mmol: 25% aq.) was added and the mixture hearted to 60° C. for 2 h. The mixture was extracted with EtOAc. The organic layer washed with brine, dried (Na₂SO₄), filtered and concentrated und reduced pressure. The product was used without further purification.

The alkylation was performed as described in US20200079776A1, the contents of which are incorporated by reference.

The following intermediates were synthesized using this method

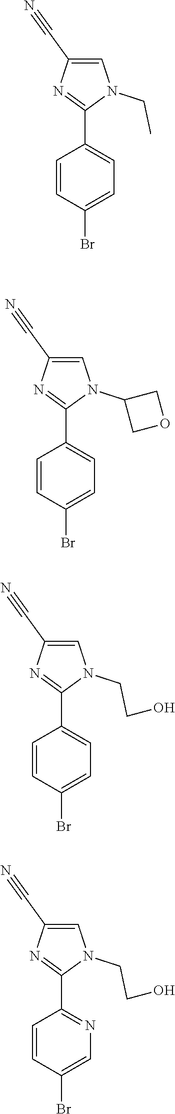 [SMILES: CCn1cc(C#N)nc1-c1ccc(Br)cc1 ; N#Cc1cn(C2COC2)c(-c2ccc(Br)cc2)n1 ; N#Cc1cn(CCO)c(-c2ccc(Br)cc2)n1 ; N#Cc1cn(CCO)c(-c2ccc(Br)cn2)n1]

4-(Aryl)-5-Alky Pyridazines

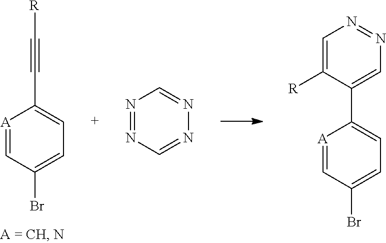

The acetylene and tetrazine were mixed in o-xylene and heated at 140° C. The desired product was isolated by evaporation and used without purification.

The following intermediates were synthesized using this method.

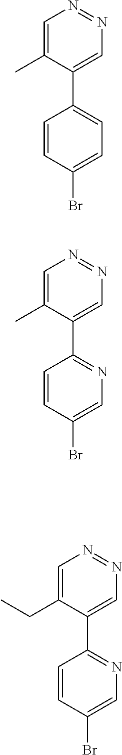

12. 5 substituted 3-(4-aryl)-1-[1,2,4]triazolo[4,3-a]pyridines

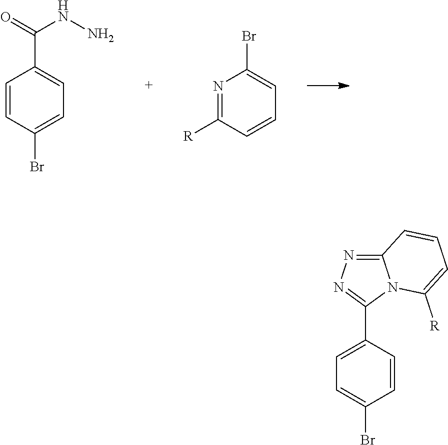

The pyridine derivative and hydrazide were dissolved in DCM, cooled to 0° C. and Tf2O (1 eq) was added slowly. The mixture was stirred for 15 min at 0° C. the at r.t. o.n. The reaction was quenched with NaHCO₃ aq. sat., extracted with DCM. The organic layer dried (Na₂SO₄), filtered and concentrated under reduced pressure. The crude product was purified by gradient flash chromatography.

The following intermediate was synthesized using this method:

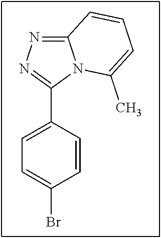 [SMILES: Cc1cccc2nnc(-c3ccc(Br)cc3)n12]

### 5 Substituted 3-(4-pyridyl)-1-[1,2,4]triazolo[4,3-a]pyridines

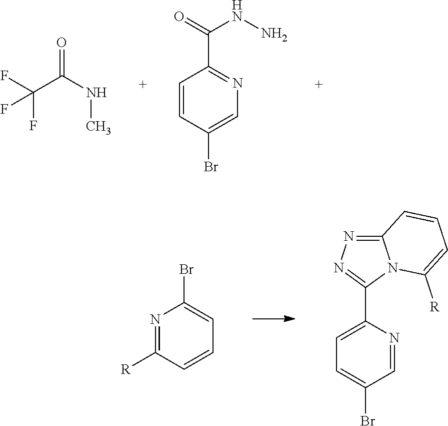

The pyridine derivative and, CF₃-aminde were dissolved in DCM, cooled to 0° C. and Tf2O (1 eq) was added slowly. The mixture was stirred for 15 min at 0° C. then hydrazide was added. The mixture was heated at 120° C. for 10 min under microwave irradiation. The reaction was quenched with NaHCO₃ aq. sat., extracted with DCM. The organic layer dried (Na₂SO₄), filtered and concentrated under reduced pressure. The crude product was purified by gradient flash chromatography.

The following intermediate was synthesized using this method:

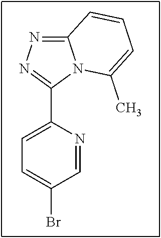 [SMILES: Cc1cccc2nnc(-c3ccc(Br)cn3)n12]

### Special Examples

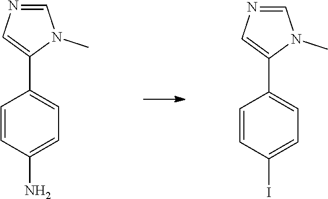

The aniline 1 eq. was dissolved in HCl conc. (3 mL/mmol) A solution of NaNO₂ (1.1 eq.) in water was added dropwise at 0° C. The solution was stirred at 0° C. for 1 h. Acetone (3 mL/mL), KI (1.1 eq) and CuI were added, and the mixture stirred at r.t. o.n. The mixture was quench by adding pH 7 buffer, extracted with EtOAc and the organic layer washed with brine, dried (Na₂SO₄), filtered and concentrated under reduced pressure. The crude product was purified by gradient flash chromatography.

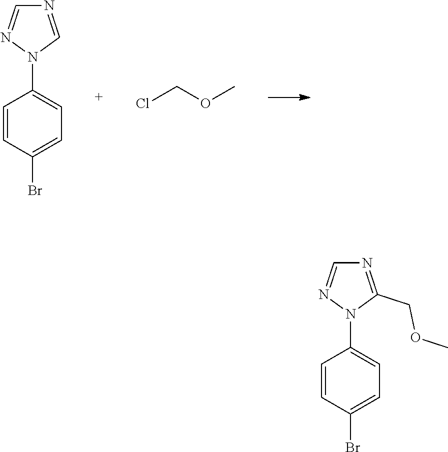

The triazole was dissolved in dry THF, cooled to −78° C. and n-BuLi (1.5 eq, 2.5M in hexanes) was added. After 0.5 h MOM-Cl was added. The mixture was allowed to warm to r.t. The reaction mixture was diluted with water and extracted with EtOAc. The organic layer was dried (Na₂SO₄), filtered and concentrated under reduced pressure. The crude product was purified by gradient flash chromatography.

Alternative Route:

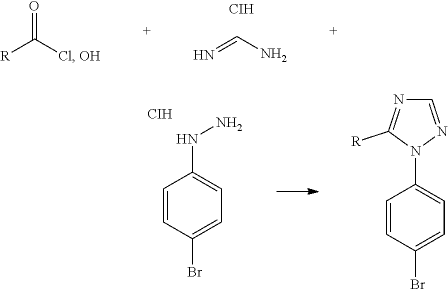

Acid chloride, amidine salt were mixed in DMF, HATU (1.1. eq.) and DIPEA (4 eq.) were added. The mixture was stirred o. n. Then AcOH (10 mL/mmol) and hydrazine salt was added. The mixture was stirred at 80° C. for 6 h, then 2 days at r.t. The reaction mixture was diluted with EtOAc, neutralized with NaHCO₃ aq. the washed with water. The organic layer is dried, (Na₂SO₄), filtered and concentrated under reduced pressure. The crude product is purified by gradient flash chromatography.

The following intermediates were synthesized following the method mentioned above.

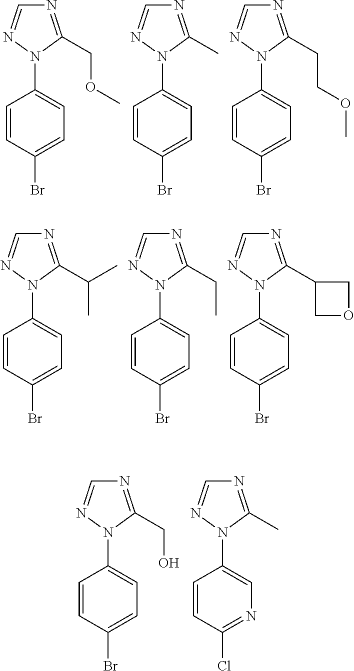

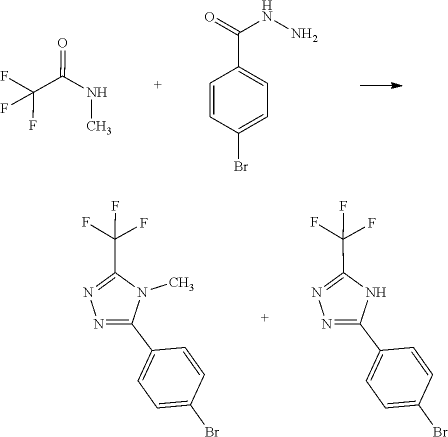

The following intermediates were synthesized as described in Org. Lett. 2015, 17, 1184-1187, the contents of which are incorporated herein by reference.

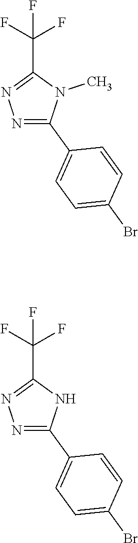 [SMILES: Cn1c(-c2ccc(Br)cc2)nnc1C(F)(F)F ; FC(F)(F)c1nnc(-c2ccc(Br)cc2)[nH]1]

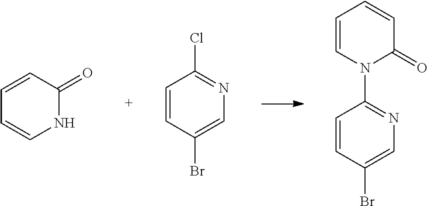 [SMILES: O=c1cccc[nH]1 ; Clc1ccc(Br)cn1 ; O=c1ccccn1-c1ccc(Br)cn1]

The amide and chloro pyridine were mixed in dry DMSO. The mixture was heated at 100° C. for 4 h. The mixture was diluted with water, extracted with EtOAc, the organic layer dried (Na₂SO₄), filtered and concentrated under reduced pressure. The crude product was purified by gradient flash chromatography.

1 Pyridyl, 5-Isopropyl Tetrazoles

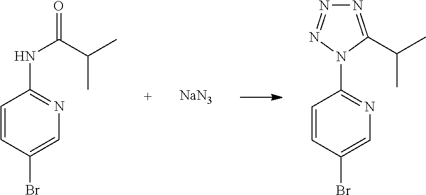

The amide was dissolved in dry THF SiCl₄ (2 eq.) and NaN₃ (2 eq.) were added. The mixture was heated at 90° C. for two days. The additional SiCl₄ and NaN₃ (2 eq. each) were and the mixture hated at 90° C. for two additional days. The mixture was diluted with NaHCO₃ aq. sat, extracted with DCM/MeOH (9/1), the organic layer washed with brine, dried (Na₂SO₄) filtered and concentrated under reduced pressure. The product was used without further purification.

14: 1-Aryl, 5-Alky 1,2,3 Triazoles

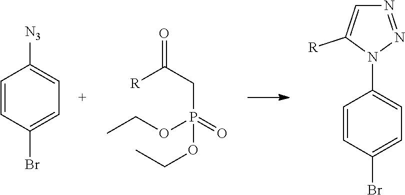

The aryl azide, tetramethylguanidine (3 eq) and the corresponding phosphonate were mixed in acetonitrile. The mixture was heated at 80° C. o.n. The mixture was diluted with water, extracted with DCM, the organic layer dried (Na₂SO₄) filtered and concentrated under reduced pressure. The crude product was used without further purification.

The following intermediated were synthesized using this procedure.

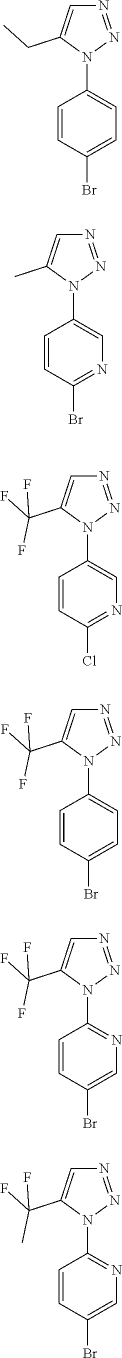

### Special Examples

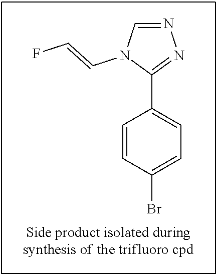 [SMILES: F/C=C/n1cnnc1-c1ccc(Br)cc1]

### 3-(bromo-aryl)-5-substituted-[1,2,4]triazolo[4,3-a]pyridines

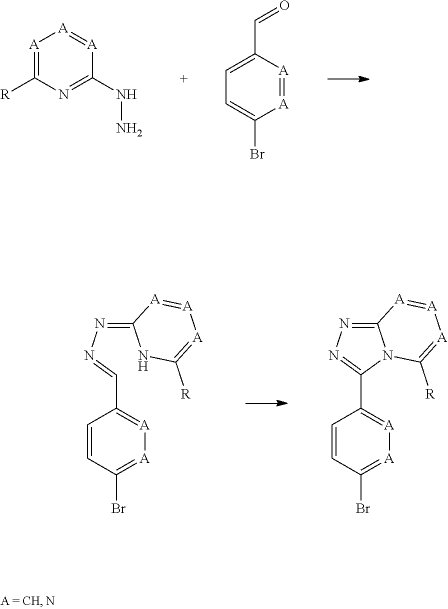

The hydrazine and aldehyde (1.1 eq.) were mixed in EtOH. The mixtures was heated for 3 h at reflux. The mixture was concentrated under reduced pressure and use without purification. The product from step 1 was dissolved in DCM, iodobenzene diacetate (1 eq.) was added and the mixture stirred at r.t.

The following intermediates were synthesized using this method:

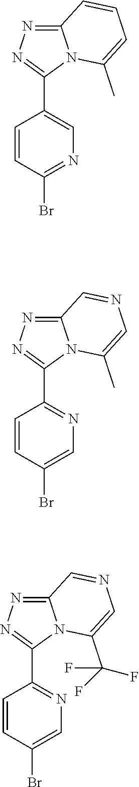 [SMILES: Cc1cccc2nnc(-c3ccc(Br)nc3)n12 ; Cc1cncc2nnc(-c3ccc(Br)cn3)n12 ; FC(F)(F)c1cncc2nnc(-c3ccc(Br)cn3)n12]

2-Alky-5-Aryl Tetrazole

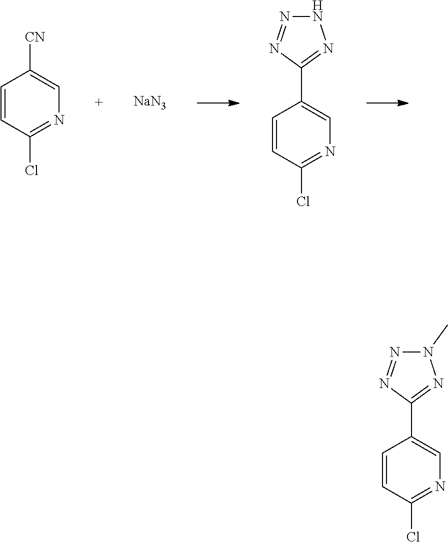

Step 1 was performed as described in J Med Chem 2010, 53, 3814-3830, the contents of which are incorporated herein by reference.

The product from step1 was dissolved in dry DMF, followed by NaH (2.3 eq. (60% suspension in oil) and Mel (1.15 eq.). The mixture was stirred o.n. at r.t., concentrated and purified with gradient flash chromatography.

### Example 8

General Synthesis Scheme to Final Compounds.

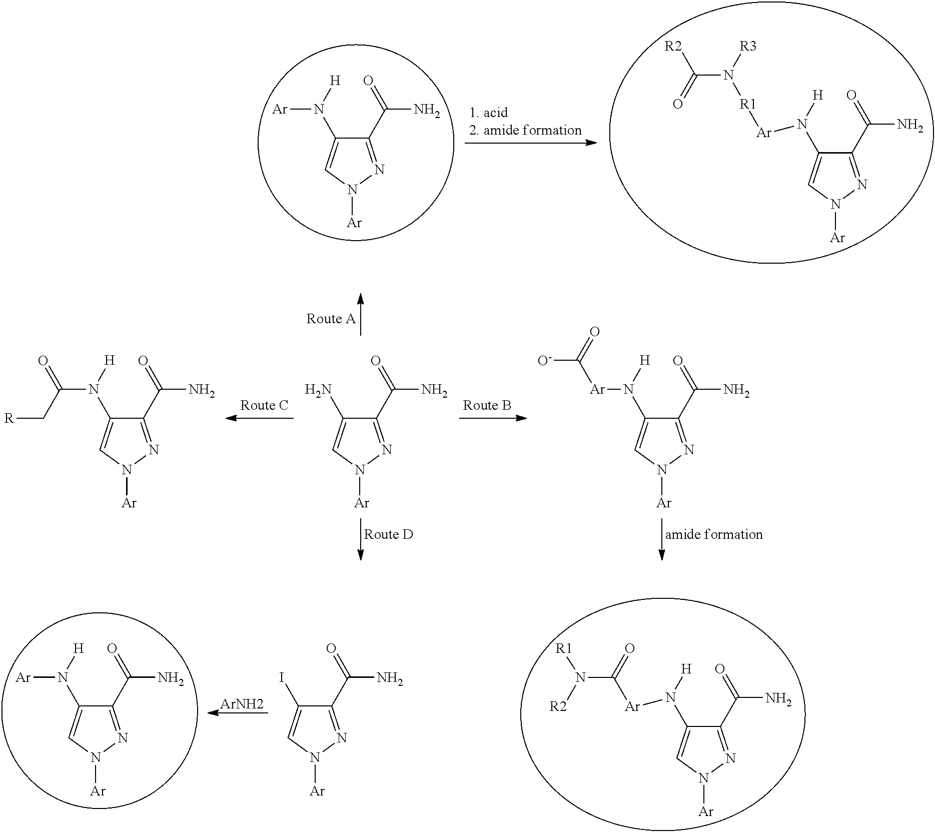

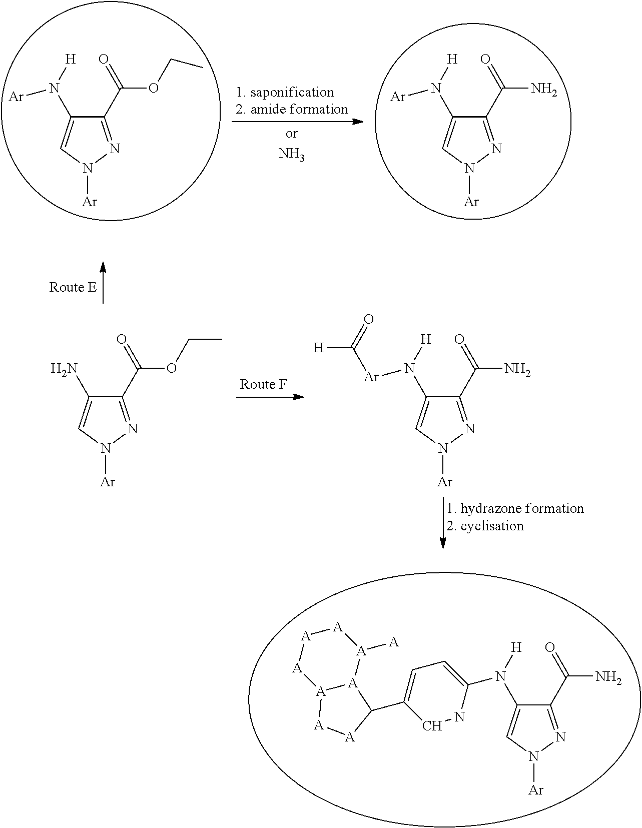

### Example 9

Route A.

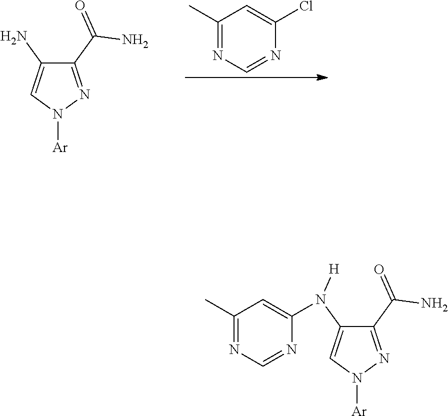

### Example 10

Method A1—arylation. The arylation were performed by nucleophilic displacement or Buchwald-Hartwig conditions.

Nucleophilic displacements—general method: The pyrazole amine and the corresponding pyrimidine chloride (2 eq.) were dissolved in iPrOH, followed by some drops of HCl 4M in dioxane. The mixture was irradiated under microwave conditions at 150° C. for 15 min. The mixture was diluted with NaHCO₃ aq. and water and extracted with ethyl acetate. The organic layer was concentrated under reduced pressure, and the crude material purified by semipreparative HPLC.

This method was used to synthesize Compound Nos. 4, 5 and 12.

### Example 11

Method A2—Buchwald type coupling:

General method A2.1: The pyrazole amine, the haloarene (2 eq.), K₂CO₃ (3 eq.) and Pd₂(dba)₃ and X-Phos were dissolved in iso-butanol. The mixture was irradiated under microwave conditions for 1 h at 110° C. The solvent was removed under reduced pressure. The residue was triturated with diethyl ether, the solids filtered off, the filtrate was concentrated under reduced pressure and purified using semipreparative HPLC.

This method was used to synthesize Compound Nos. 6, 7, 8, 9, 10, 11, 17, 18, 19, 20, 21, 22, 28, and 29.

### Example 12

General method A2.2: The pyrazole amine, the haloarene (1.2 eq.), NaOtBu (3 eq.) and X-Phos Pd G2 (0.1 eq.), X-Phos (0.1. eq.) were dissolved in dioxane. The mixture was degassed with N₂, then heated to 95° C. o.n. The mixture was filtered, and the filtrate concentrated under reduced pressure. The residue was purified by chromatography.

This method was used to synthesize Compound Nos. 31, 32, 33, 35, 36, 37, 41, 42, 43, 44, 45, 46, 47, 48, 49, 50, 51, 52, 53, 54, 56, 60, 61, 67, 74, 82, 83, 84, 85, 86, 89, 90, 91, 92, 93, 94, 95, 204, 246, 247, 248, 313, 314, 315, 316, 323, 324, 325, 331, 332, 354, 355, 356, 358, 362, 363, 364, 365, 366, 367, 369, 370, 382, 383, 407, 439, 440, 441, 442, 443, and 444.

### Example 13

General method A2.3: The pyrazole amine, the haloarene (1.2 eq.), NaOtBu (3 eq.) and ^{t}BuBrettPhos Pd G3 (0.25 eq.), X-Phos (0.25. eq.) were dissolved in dioxane. The mixture was degassed with N₂, then heated to 95° C. for 3 h. The mixture was filtered, and the filtrate concentrated under reduced pressure. The residue was purified by chromatography.

This method was used to synthesize Compound Nos. 55, 57, 58, 59, 62, 63, 65, 66, 68, 69, 70, 71, 72, 73, 75, 76, 77, 78, 79, 80, 81, 87, 88, 96, 201, 202, 203, 205, 206, 207, 208, 210, 211, 212, 213, 214, 215, 216, 217, 218, 219, 220, 221, 222, 223, 224, 225, 227, 228, 229, 230, 231, 232, 233, 234, 235, 236, 237, 238, 239, 240, 241, 242, 243, 244, 245, 249, 250, 251, 252, 253, 254, 255, 256, 257, 258, 259, 260, 261, 262, 263, 264, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 320, 321, 322, 326, 327, 328, 329, 330, 333, 334, 335, 336, 337, 338, 339, 340, 341, 342, 343, 344, 345, 346, 347, 348, 349, 350, 351, 352, 353, 361, 362, 373, 375, 376, 381, 384, 385, 386, 387, 388, 389, 390, 391, 393, 394, 395, 398, 399, 400, 401, 402, 403, 404, 405, 406, 408, 409, 410, 411, 415, 419, 420, 421, 422, 423, 424, 425, 426, 427, 428, 429, 430, 431, 432, 445, 446, 448, 449, 451, 452, 454, 456, 459, 460, 462, 463, 464, 465, 466, 467, 468, 473, and 477.

### Example 14

Further Extension of Route A: Example of Boc Deprotection:

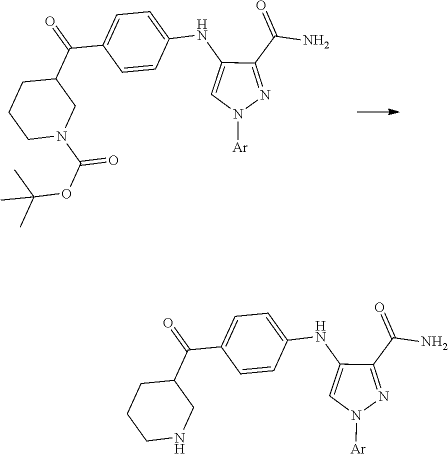

General method: The protected compound was dissolved in DCM. TFA (10% v/v) was added and the mixture stirred for 1 h. The mixture was diluted with water, basified with NaHCO₃ (pH8), dried over Na₂SO₄, filtered and concentrated und reduced pressure.

This method was used to synthesize Compound Nos. 38 39, 317, 318, 319, 357, and 368.

### Special Example

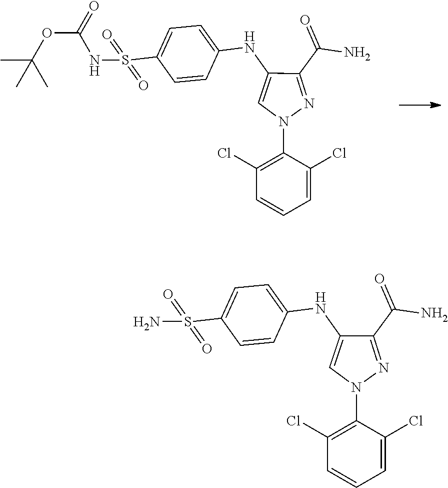 [SMILES: CC(C)(C)OC(=O)NS(=O)(=O)c1ccc(Nc2cn(-c3c(Cl)cccc3Cl)nc2C(N)=O)cc1 ; NC(=O)c1nn(-c2c(Cl)cccc2Cl)cc1Nc1ccc(S(N)(=O)=O)cc1]

The protected compound was dissolved in DCM. TFA (10% v/v) was added and the mixture stirred for 1 h. The mixture was diluted with water, basified with NaHCO₃ (pH8), dried over Na₂SO₄, filtered and concentrated und reduced pressure and purified by reversed phase HPLC.

### Example 15

Route B: The following intermediates was synthesized using a Buchwald type—Method A2:

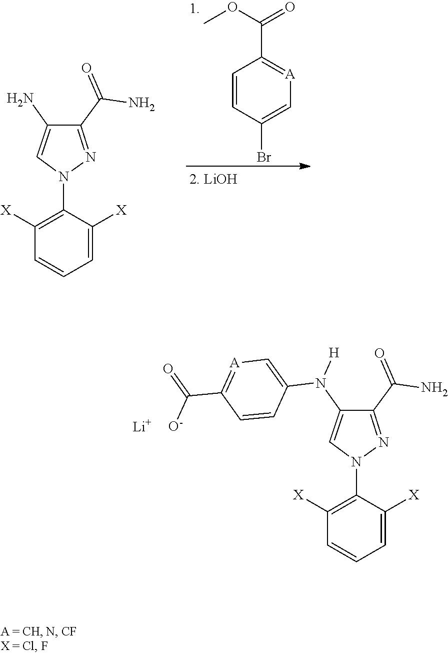

Modified work up: The solvent was removed by concentration under reduced pressure. The residue partitioned in between DCM and water, the aqueous layer was washed with DCM (3×) and 10% MeOH in DCM. The combined organic layers were washed with bine, dried (Na₂SO₄) and concentrated under reduced pressure. The crude product was purified by gradient flash chromatography. The intermediate ester was saponified with LiOH (10 eq) in THF:MeOH (3:1) at 60° C. o.n. The mixture was concentrated and used without further purification.

Amination:

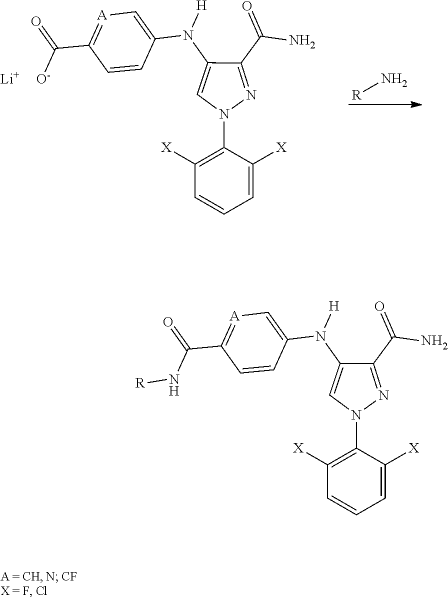

General method: The lithium salt was dissolved in DMF, the corresponding amine (2 eq.), HATU (3 eq.) and DIPEA (3 eq.) were added. The mixture was stirred at 50° C. o.n. The reaction mixture was purified without work up with semipreparative HPLC.

This method was used to synthesize Compound Nos. 13, 14, 15, 16, 23, 24, 25, 26, 27, 30, 34, 40, 226, and 472

Special Method:

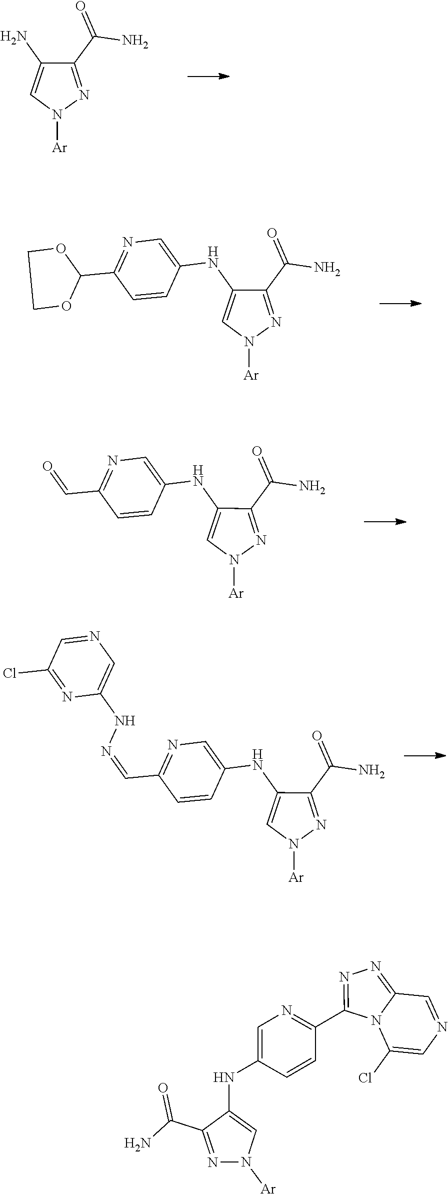

Step 1 is performed according to the procedures of A2.3. The deprotection is performed by treating the product of step1 with THF/HCl aq. 2M for 1 h at r.t. The mixture was diluted with water extracted with DCM, the organics dried (Na₂SO₄), filtered and concentrated under reduced pressure. Hydrazone formation and oxidative cyclisation are following the procedures of intermediate 15 synthesis. The final crude product is purified by reversed phase chromatography

This method was used to synthesize Compound Nos. 447, 470, 471, 474, 475, and 476.

### Example 16

Route C—Amide Formation.

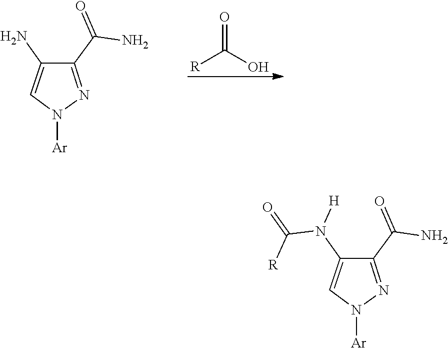

General method: The pyrazole amine and carboxylic acid (1 eq.) were dissolved in DMF (dry), followed by HOBt (2 eq), EDCI (2 eq.) and DIPEA (3 eq.). The mixture was stirred at r.t. for 4 h. The mixture was diluted with ethyl acetate, washed with NaHCO₃ aq. and brine. The desired compound was purified by semipreparative HPLC.

Alternatively the corresponding acid chloride was used with triethyl amine (3 eq.) in DCM.

The following compounds were synthesized according this procedure using an appropriate pyrazole amine and carboxylic acid.

This method was used to synthesize Compound Nos. 1, 2, and 3.

### Example 17

Route D—Special Final Compounds.

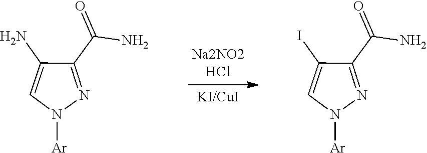

A solution of NaNO₂ (1.2 eq) was dropped to a cooled (0° C.) solution of the aminopyrazole in HCl (37% aq). Subsequently acetone, KI (1.1 eq.) and CuI (1.1 eq.) were added. The solution was stirred at r.t. o.n. The reaction mixture was quenched with buffer (pH7). The aqueous layer was extracted with DCM and 5% MeOH in DCM. The combined organic phases were dried (Na₂SO₄) filtered and evaporated under reduced pressure. The residue was purified by gradient flash chromatography.

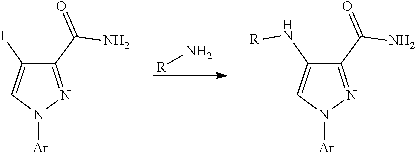

A modified method B was used to synthesize Compound No. 64.

Compound No. 453 was isolated as a byproduct in the corresponding nitril synthesis.

Reductive amination was used to synthesize Compound No. 455.

The aldehyde was dissolved in MeOH, followed by the addition of amine, acetic acid and sodiumcyanoborohydride (5 eq. each). After 30 min the mixture was dilute with water, extracted with DCM, dried (Na₂SO₄), filtered and concentrated under reduced pressure. The crude product was purified by reversed phase HPLC.

Route E:

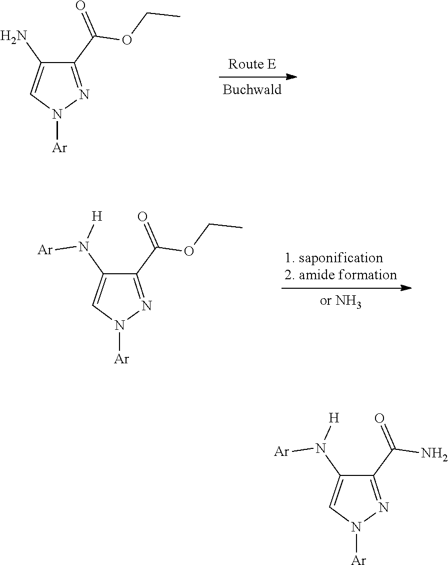

The “Buchwald-type” step was performed, using the conditions A2.1, A2.2 or A2.3 starting from the ester intermediate of the building block synthesis.

The saponification is performed by treating the corresponding ester by NaOH in MeOH. The amidation was performed by dissolving the acid in DMF, followed by addition of an excess (10-20 eq.) ammonium chloride, HATU (1.2 eq.) and DIPEA (3 eq.)

Alternatively the ester intermediate can be treated with NH₃ to get directly to the amide.

The residue was purified by chromatography.

The method of using A2.3 in the Buchwald step was used to synthesize Compound Nos. 359, 372, 374, 375, 378, 379, 380, 392, 396, 397, 412, 413, 414, 416, 417, 418. and 461

This method can also be used to make the following compounds:

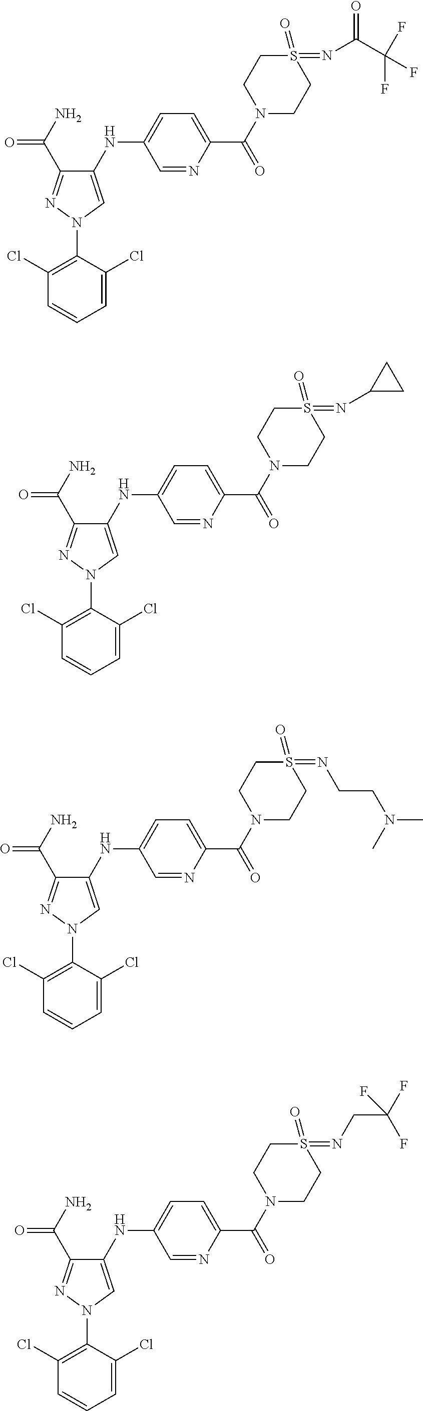 [SMILES: NC(=O)c1nn(-c2c(Cl)cccc2Cl)cc1Nc1ccc(C(=O)N2CCS(=O)(=NC(=O)C(F)(F)F)CC2)nc1 ; NC(=O)c1nn(-c2c(Cl)cccc2Cl)cc1Nc1ccc(C(=O)N2CCS(=O)(=NC3CC3)CC2)nc1 ; CN(C)CCN=S1(=O)CCN(C(=O)c2ccc(Nc3cn(-c4c(Cl)cccc4Cl)nc3C(N)=O)cn2)CC1 ; NC(=O)c1nn(-c2c(Cl)cccc2Cl)cc1Nc1ccc(C(=O)N2CCS(=O)(=NCC(F)(F)F)CC2)nc1]

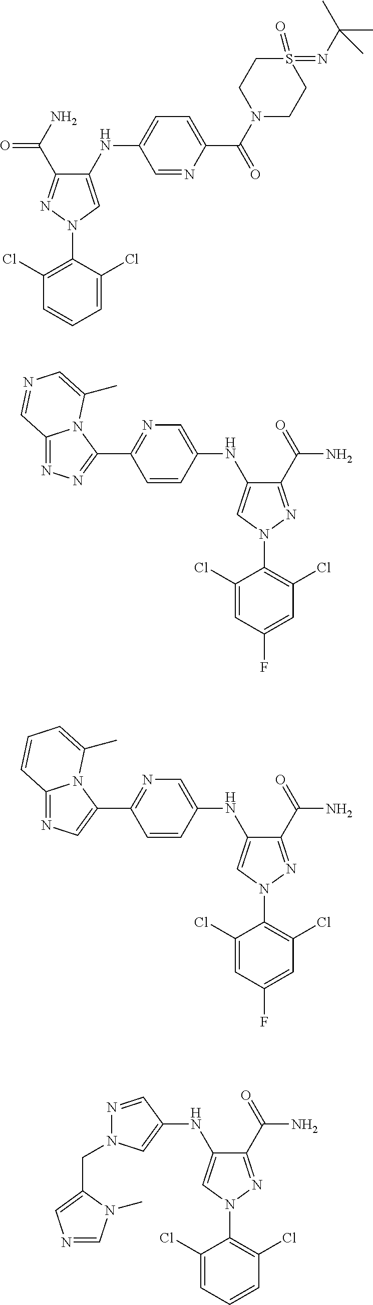 [SMILES: CC(C)(C)N=S1(=O)CCN(C(=O)c2ccc(Nc3cn(-c4c(Cl)cccc4Cl)nc3C(N)=O)cn2)CC1 ; Cc1cncc2nnc(-c3ccc(Nc4cn(-c5c(Cl)cc(F)cc5Cl)nc4C(N)=O)cn3)n12 ; Cc1cccc2ncc(-c3ccc(Nc4cn(-c5c(Cl)cc(F)cc5Cl)nc4C(N)=O)cn3)n12 ; Cn1cncc1Cn1cc(Nc2cn(-c3c(Cl)cccc3Cl)nc2C(N)=O)cn1]

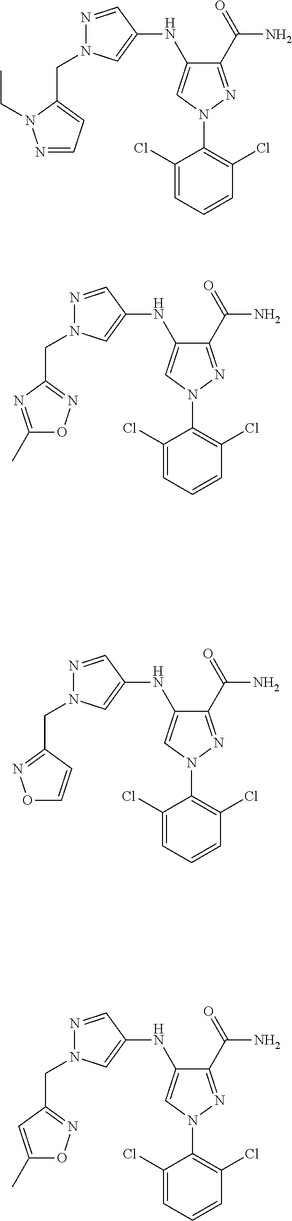 [SMILES: CCn1nccc1Cn1cc(Nc2cn(-c3c(Cl)cccc3Cl)nc2C(N)=O)cn1 ; Cc1nc(Cn2cc(Nc3cn(-c4c(Cl)cccc4Cl)nc3C(N)=O)cn2)no1 ; NC(=O)c1nn(-c2c(Cl)cccc2Cl)cc1Nc1cnn(Cc2ccon2)c1 ; Cc1cc(Cn2cc(Nc3cn(-c4c(Cl)cccc4Cl)nc3C(N)=O)cn2)no1]

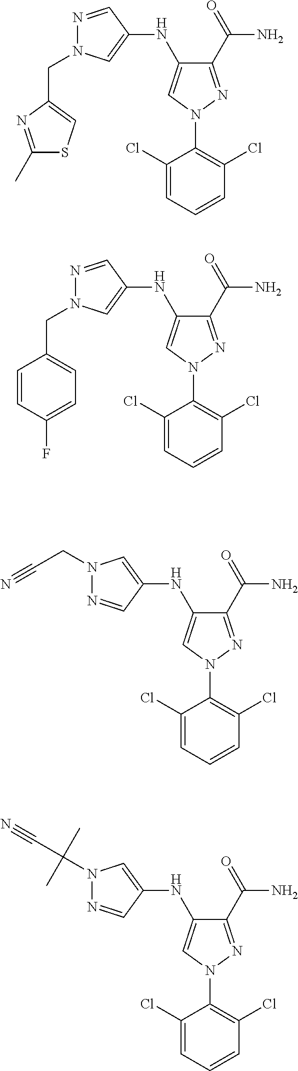

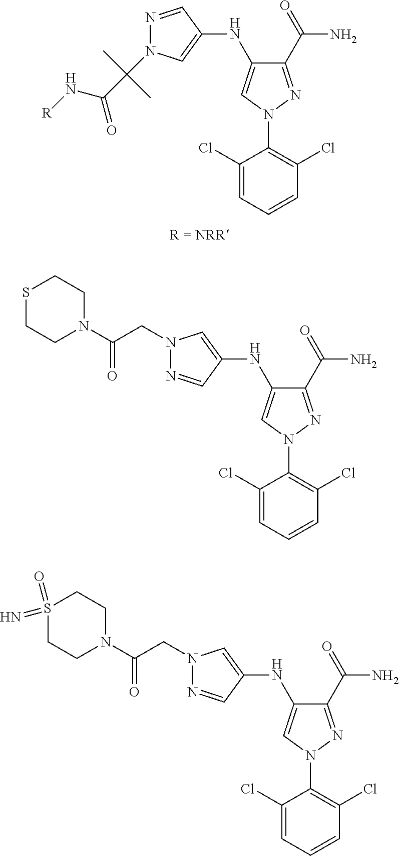

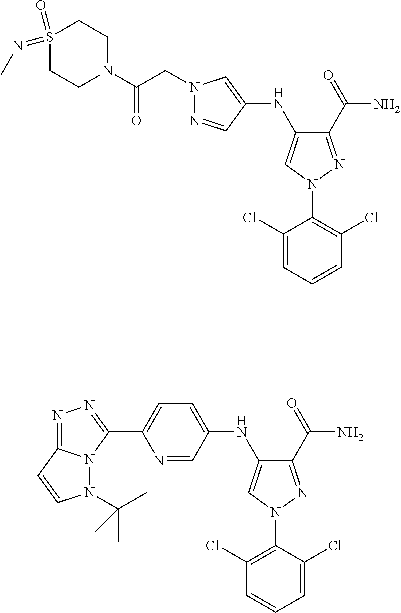 [SMILES: CN=S1(=O)CCN(C(=O)Cn2cc(Nc3cn(-c4c(Cl)cccc4Cl)nc3C(N)=O)cn2)CC1 ; CC(C)(C)n1ccc2nnc(-c3ccc(Nc4cn(-c5c(Cl)cccc5Cl)nc4C(N)=O)cn3)n21]

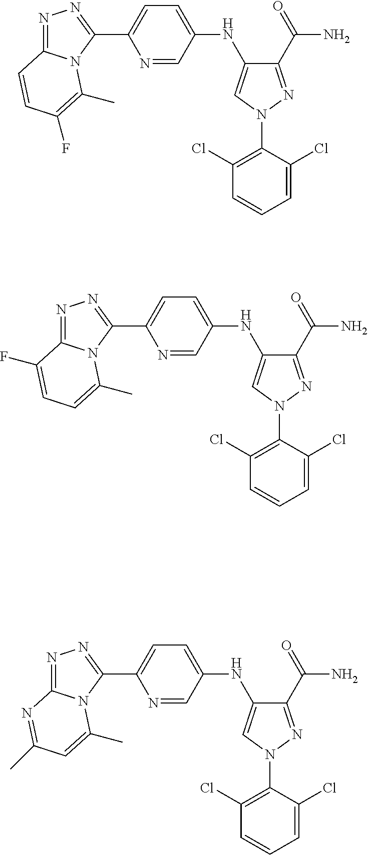 [SMILES: Cc1c(F)ccc2nnc(-c3ccc(Nc4cn(-c5c(Cl)cccc5Cl)nc4C(N)=O)cn3)n12 ; Cc1ccc(F)c2nnc(-c3ccc(Nc4cn(-c5c(Cl)cccc5Cl)nc4C(N)=O)cn3)n12 ; Cc1cc(C)n2c(-c3ccc(Nc4cn(-c5c(Cl)cccc5Cl)nc4C(N)=O)cn3)nnc2n1]

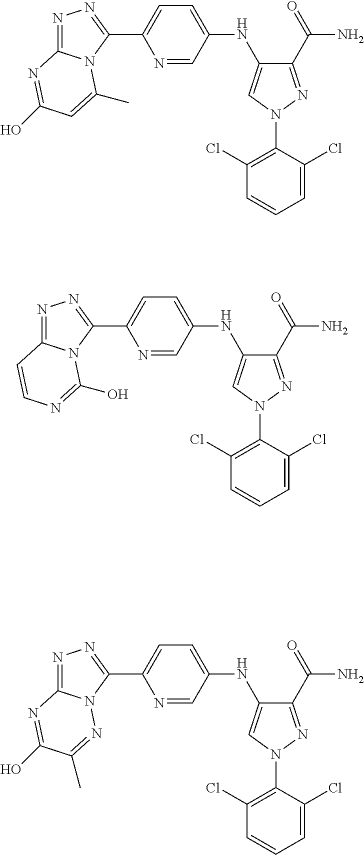 [SMILES: Cc1cc(O)nc2nnc(-c3ccc(Nc4cn(-c5c(Cl)cccc5Cl)nc4C(N)=O)cn3)n12 ; NC(=O)c1nn(-c2c(Cl)cccc2Cl)cc1Nc1ccc(-c2nnc3ccnc(O)n23)nc1 ; Cc1nn2c(-c3ccc(Nc4cn(-c5c(Cl)cccc5Cl)nc4C(N)=O)cn3)nnc2nc1O]

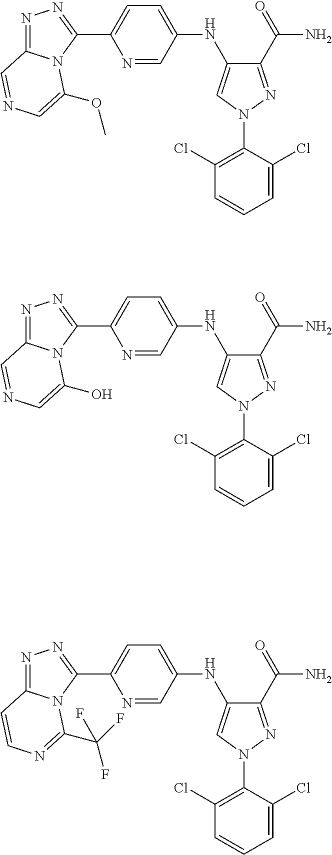 [SMILES: COc1cncc2nnc(-c3ccc(Nc4cn(-c5c(Cl)cccc5Cl)nc4C(N)=O)cn3)n12 ; NC(=O)c1nn(-c2c(Cl)cccc2Cl)cc1Nc1ccc(-c2nnc3cncc(O)n23)nc1 ; NC(=O)c1nn(-c2c(Cl)cccc2Cl)cc1Nc1ccc(-c2nnc3ccnc(C(F)(F)F)n23)nc1]

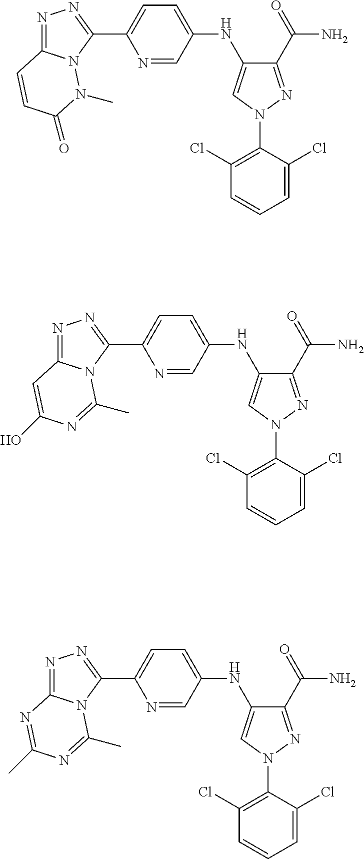 [SMILES: Cn1c(=O)ccc2nnc(-c3ccc(Nc4cn(-c5c(Cl)cccc5Cl)nc4C(N)=O)cn3)n21 ; Cc1nc(O)cc2nnc(-c3ccc(Nc4cn(-c5c(Cl)cccc5Cl)nc4C(N)=O)cn3)n12 ; Cc1nc(C)n2c(-c3ccc(Nc4cn(-c5c(Cl)cccc5Cl)nc4C(N)=O)cn3)nnc2n1]

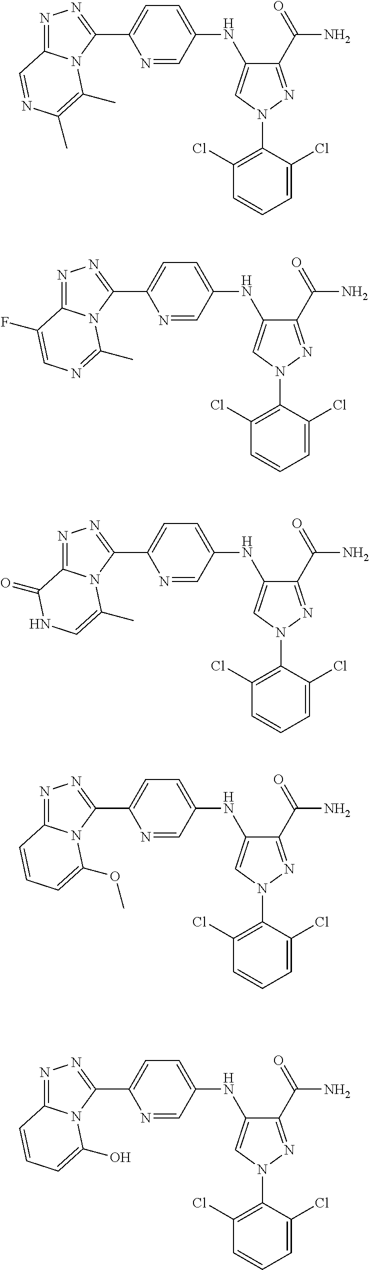 [SMILES: Cc1ncc2nnc(-c3ccc(Nc4cn(-c5c(Cl)cccc5Cl)nc4C(N)=O)cn3)n2c1C ; Cc1ncc(F)c2nnc(-c3ccc(Nc4cn(-c5c(Cl)cccc5Cl)nc4C(N)=O)cn3)n12 ; Cc1c[nH]c(=O)c2nnc(-c3ccc(Nc4cn(-c5c(Cl)cccc5Cl)nc4C(N)=O)cn3)n12 ; COc1cccc2nnc(-c3ccc(Nc4cn(-c5c(Cl)cccc5Cl)nc4C(N)=O)cn3)n12 ; NC(=O)c1nn(-c2c(Cl)cccc2Cl)cc1Nc1ccc(-c2nnc3cccc(O)n23)nc1]

### Example 18

The HPLC Methods used in preparation of the compounds are listed in Table 6.

TABLE 6

| | | | flow | |
|---|---|---|---|---|
| Method | col | grad | (mL/min) | ms |
| HPLC-MS A | Waters ACQUITY UPLC HSS T3 | 2%-4.0 min->95%-1.0 min->95%-0.1 min->2%- | 0.6 | ESI positive |
| | 50 × 2.1 1.8 μm PN: 186003538 | 2.9 min->2%; ACN/H₂O + 0.1% HCOOH | | & negative |
| HPLC-MS B | Waters ACQUITY UPLC CSH | 5%-4.0 min->95%-1.0 min->95%-0.1 min->5%- | 0.6 | ESI positive |
| | C18 50 × 2.1 1.7 μm | 2.9 min->5%; ACN/H2O + 0.1% HCOOH | | & negative |
| | PN: 186005296 | | | |
| HPLC-MS C | Waters ACQUITY UPLC CSH | 4%-4.0 min->96%-1.0 min->96%-0.1 min->4%- | 1.2 | ESI positive |
| | C18 50 × 2.1 1.7 μm | 2.9 min->4%; ACN/H2O + 100 mM NH4Ac- | | |
| | PN: 186005296 | | | |
| HPLC-MS D | Waters CORTECS UPLC C18+ | 5%-4.0 min->95%-1.0 min->95%-0.1 min->5%- | 0.6 | ESI positive |
| | 50 × 2.1 1.6 μm PN: 186007114 | 2.9 min->5%; ACN/H2O + 0.1% HCOOH | | & negative |
| HPLC-MS E | Waters ACQUITY UPLC CSH | 5%-0.75 min->95%-0.5 min->95%-0.05 min->5%- | 1.2 | ESI positive |
| | C18 50 × 2.1 1.7 μm PN: 186005296 | 1.2 min->5%; ACN/H2O + 0.1% HCOOH | | |

### Example 19

Chemical analytical data for the compounds listed in Table 1 is summarized in Table 7.

TABLE 7

| Compound | HPLC-MS | retention | m + H | |
|---|---|---|---|---|
| No | Method | time ( min) | Found | NMR |
| 1 | C | 3.047 | 360.2 | 1H NMR (400 MHz, MeOD) δ 8.70 (s, 1H), 7.79-7.73 (m, 2H), 7.12-7.03 (m, 2H), |
| | | | | 3.87 (m, 7H), 3.25 (s, 2H), 2.66 (m, 4H). |
| 2 | C | 2.923 | 366.2 | 1H NMR (400 MHz, Methanol-d₄) δ 8.42 (d, J = 1.2 Hz, 1H), 7.43 (tt, J = 8.3, 6.0 |
| | | | | Hz, 1H), 7.15-7.04 (m, 2H), 3.90 (s, 4H), 3.51 (d, J = 5.0 Hz, 7H), 2.86 (s, 3H). |
| 3 | C | 3.015 | 348.1 | 1H NMR (400 MHz, Methanol-d₄) δ 8.72 (d, J = 2.9 Hz, 1H), 7.92 (dtd, J = 15.8, |
| | | | | 7.9, 1.6 Hz, 2H), 7.67 (d, J = 3.0 Hz, 1H), 7.51-7.43 (m, 1H), 7.43-7.28 (m, 5H), |
| | | | | 3.91-3.82 (m, 4H), 3.24 (s, 2H), 2.65 (dd, J = 5.5, 3.5 Hz, 4H). |
| 4 | C | 3.074 | 331.2 | 1H NMR (400 MHz, Chloroform-d) δ 9.37 (s, 1H), 8.72 (s, 1H), 8.69 (q, J = 1.0 Hz, |
| | | | | 1H), 7.48 (tt, J = 8.6, 6.0 Hz, 1H), 7.21-7.09 (m, 2H), 6.85 (s, 1H), 6.63 (s, |
| | | | | 1H), 5.56 (s, 1H), 4.14 (q, J = 7.2 Hz, 1H), 2.50 (s, 3H), 2.07 (s, 2H), 1.28 (t, |
| | | | | J = 7.2 Hz, 3H). |
| 5 | C | 3.174 | 313.2 | 1H NMR (400 MHz, Chloroform-d) δ 8.93 (d, J = 3.0 Hz, 1H), 8.74 (s, 1H), 7.83 |
| | | | | (td, J = 7.8, 2.1 Hz, 1H), 7.47-7.35 (m, 1H), 7.35-7.25 (m, 3H), 6.60 (s, 1H), |
| | | | | 2.48 (s, 3H). |
| 6 | C | 2.690 | 331.2 | |
| 7 | C | 3.642 | 316.2 | 1H NMR (400 MHz, Methanol-d₄) δ 8.62 (t, J = 1.1 Hz, 1H), 8.20 (ddd, J = 5.1, |
| | | | | 1.9, 0.9 Hz, 1H), 7.62-7.51 (m, 2H), 7.29-7.19 (m, 2H), 6.84 (dt, J = 8.4, 1.0 |
| | | | | Hz, 1H), 6.74 (ddd, J = 7.2, 5.1, 0.9 Hz, 1H). |
| 8 | C | 2.822 | 346.2 | |
| 9 | C | 4.017 | 372.3 | 1H NMR (400 MHz, Methanol-d₄) δ 8.73 (s, 1H), 7.71 (dt, J = 7.6, 1.0 Hz, 1H), |
| | | | | 7.65-7.56 (m, 2H), 7.37-7.22 (m, 3H), 7.16 (td, J = 7.7, 1.2 Hz, 1H). |
| 10 | C | 3.834 | 330.3 | 1H NMR (400 MHz, Methanol-d₄) δ 8.59 (t, J = 1.0 Hz, 1H), 8.05 (dd, J = 5.2, 0.8 |
| | | | | Hz, 1H), 7.62-7.49 (m, 1H), 7.30-7.11 (m, 2H), 6.67 (dt, J = 1.6, 0.9 Hz, 1H), |
| | | | | 6.61 (ddd, J = 5.3, 1.5, 0.7 Hz, 1H), 2.29 (s, 3H). |
| 11 | C | 3.474 | 355.3 | 1H NMR (400 MHz, Methanol-d₄) δ 8.52 (d, J = 1.0 Hz, 1H), 7.60 (tt, J = 8.6, 6.1 |
| | | | | Hz, 1H), 7.36-7.22 (m, 4H), 7.06 (dd, J = 5.9, 3.2 Hz, 2H). |
| 12 | C | 3.118 | 428.3 | 1H NMR (400 MHz, Methanol-d₄) δ 8.15 (s, 1H), 7.63-7.52 (m, 1H), 7.41-7.36 (m, |
| | | | | 2H), 7.28-7.18 (m, 2H), 7.10-7.04 (m, 2H), 3.82-3.53 (brs, 8H). |
| 13 | C | 3.120 | 442.2 | 1H NMR (400 MHz, Methanol-d₄) δ 8.19 (s, 1H), 7.83-7.75 (m, 2H), 7.59 (ddd, J = |
| | | | | 8.6, 6.1, 2.5 Hz, 1H), 7.30-7.21 (m, 2H), 7.09-7.01 (m, 2H), 4.09 (ddd, J = |
| | | | | 11.3, 7.0, 4.3 Hz, 1H), 4.02-3.94 (m, 2H), 3.52 (td, J = 11.9, 2.1 Hz, 2H), 1.89 |
| | | | | (ddd, J = 12.6, 4.4, 2.0 Hz, 2H), 1.74-1.60 (m, 2H). |
| 14 | C | 3.467 | 440.2 | 1H NMR (400 MHz, Methanol-d₄) δ 8.21 (s, 1H), 7.85-7.79 (m, 2H), 7.64-7.53 (m, |
| | | | | 1H), 7.30-7.21 (m, 2H), 7.11-7.04 (m, 2H), 4.07 (q, J = 9.4 Hz, 1H). |
| 15 | C | 3.634 | 414.2 | 1H NMR (400 MHz, Methanol-d₄) δ 8.17 (s, 1H), 7.74-7.68 (m, 2H), 7.63-7.54 (m, |
| | | | | 1H), 7.30-7.20 (m, 2H), 7.07-6.98 (m, 2H), 1.45 (s, 9H). |
| 16 | C | 3.158 | 386.2 | 1H NMR (400 MHz, Methanol-d₄) δ 8.19 (s, 1H), 7.81-7.74 (m, 2H), 7.61-7.57 (m, |
| | | | | 1H), 7.25 (t, J = 8.3 Hz, 2H), 7.09-7.02 (m, 2H), 3.39 (q, J = 7.3 Hz, 3H), 1.21 |
| | | | | (t, J = 7.2 Hz, 4H). |
| 17 | C | 3.360 | 460.2 | 1H NMR (400 MHz, Methanol-d₄) d 8.12 (s, 1H), 7.64 (d, J = 1.5 Hz, 1H), 7.62 (d, |
| | | | | J = 0.7 Hz, 1H), 7.55 (dd, J = 9.2, 7.0 Hz, 1H), 7.40 (d, J = 8.6 Hz, 2H), 7.08 |
| | | | | (d, J = 8.6 Hz, 2H), 3.68 (bs, 8H). |
| 18 | C | 3.369 | 428.2 | 1H NMR (400 MHz, Methanol-d₄) δ 8.17 (s, 1H), 7.64-7.54 (m, 1H), 7.36 (t, J = 7.8 |
| | | | | Hz, 1H), 7.30-7.19 (m, 2H), 7.16-7.09 (m, 1H), 7.08-7.04 (m, 1H), 6.87 (dt, J = |
| | | | | 7.7, 1.1 Hz, 1H), 3.75 (brs, 5H), 3.62 (brs, 3H). |
| 19 | C | 3.904 | 349.3 | |
| 20 | C | 4.208 | 381.0 | 1H NMR (400 MHz, Methanol-d₄) d 8.03 (s, 1H), 7.67-7.61 (m, 2H), 7.56 (dd, J = |
| | | | | 9.2, 7.0 Hz, 1H), 7.30-7.21 (m, 2H), 7.05-6.98 (m, 2H). |
| 21 | C | 3.950 | 347.0 | 1H NMR (400 MHz, Methanol-d₄) d 8.00 (s, 1H), 7.64 (dd, J = 8.1, 1.1 Hz, 2H), |
| | | | | 7.56 (dd, J = 9.2, 7.0 Hz, 1H), 7.32-7.23 (m, 2H), 7.07-6.99 (m, 3H), 6.85 (tt, |
| | | | | J = 7.4, 1.1 Hz, 1H). |
| 22 | C | 3.335 | 444.2 | 1H NMR (400 MHz, Methanol-d₄) d 8.15 (s, 1H), 7.59 (td, J = 8.4, 5.6 Hz, 1H), |
| | | | | 7.50 (dt, J = 8.3, 1.4 Hz, 1H), 7.43-7.34 (m, 3H), 7.11-7.04 (m, 2H), 3.66 (d, J = |
| | | | | 14.9 Hz, 8H). |
| 23 | C | 3.685 | 416.2 | |
| 24 | C | 3.185 | 387.2 | |
| 25 | C | 3.361 | 387.2 | |
| 26 | C | 3.801 | 446.2 | 1H NMR (400 MHz, Methanol-d₄) d 8.12 (s, 1H), 7.75-7.68 (m, 2H), 7.66-7.60 (m, |
| | | | | 2H), 7.56 (dd, J = 9.2, 7.0 Hz, 1H), 7.08-6.99 (m, 2H), 1.44 (s, 9H). |
| 27 | C | 3.719 | 430.2 | 1H NMR (400 MHz, Methanol-d₄) d 8.15 (s, 1H), 7.74-7.67 (m, 2H), 7.59 (td, J = |
| | | | | 8.3, 5.6 Hz, 1H), 7.50 (dt, J = 8.2, 1.3 Hz, 1H), 7.38 (ddd, J = 9.6, 8.5, 1.4 |
| | | | | Hz, 1H), 7.03 (d, J = 8.8 Hz, 2H), 1.44 (s, 9H). |
| 28 | C | 4.028 | 464.0 | 1H NMR (400 MHz, Methanol-d₄) d 8.19 (s, 1H), 7.70-7.63 (m, 3H), 7.58 (dd, J = |
| | | | | 9.2, 6.9 Hz, 1H), 6.89 (dd, J = 8.6, 2.3 Hz, 1H), 6.80 (dd, J = 14.0, 2.3 Hz, |
| | | | | 1H), 1.46 (s, 9H). |
| 29 | C | 3.939 | 448.2 | 1H NMR (400 MHz, Methanol-d₄) d 8.23 (s, 1H), 7.67 (t, J = 8.7 Hz, 1H), 7.61 (td, |
| | | | | J = 8.3, 5.6 Hz, 1H), 7.52 (dt, (J = 8.3, 1.3 Hz, 1H), 7.40 (ddd, J = 9.6, 8.4, |
| | | | | 1.4 Hz, 1H), 6.89 (dd, J = 8.6, 2.3 Hz, 1H), 6.80 (dd, J = 14.0, 2.3 Hz, 1H), |
| | | | | 1.46 (s, 9H). |
| 30 | C | 2.884 | 429.2 | 1H NMR (400 MHz, Methanol-d₄) δ 8.34-8.32 (m, 1H), 8.28 (s, 1H), 7.65-7.50 (m, |
| | | | | 3H), 7.26 (t, J = 8.3 Hz, 2H), 3.83-3.63 (brs, 8H). |
| 31 | C | 2.903 | 487.2 | 1H NMR (400 MHz, Methanol-d₄) d 8.15 (s, 1H), 7.85-7.74 (m, 2H), 7.64 (d, J = 8.8 |
| | | | | Hz, 2H), 7.56 (dd, J = 9.2, 6.9 Hz, 1H), 7.12-7.03 (m, 2H), 3.99 (tt, J = 11.0, |
| | | | | 4.1 Hz, 1H), 3.18 (dd, J = 12.0, 3.6 Hz, 2H), 2.54 (s, 5H), 2.12-1.99 (m, 2H), |
| | | | | 1.80 (qd, J = 11.6, 6.2 Hz, 2H). |
| 32 | C | 3.073 | 473.2 | 1H NMR (400 MHz, Methanol-d₄) d 8.12 (s, 1H), 7.66-7.62 (m, 2H), 7.56 (dd, J = |
| | | | | 9.2, 7.0 Hz, 1H), 7.39 (d, J = 8.7 Hz, 2H), 7.08 (d, J = 8.7 Hz, 2H), 3.68 (s, |
| | | | | 4H), 2.52 (s, 4H), 2.36 (s, 3H). |
| 33 | C | 3.968 | 573.2 | 1H NMR (400 MHz, Methanol-d₄) d 8.17 (s, 1H), 7.80 (d, J = 8.7 Hz, 2H), 7.68-7.63 |
| | | | | (m, 2H), 7.58 (dd, J = 9.2, 7.0 Hz, 1H), 7.11-7.05 (m, 2H), 3.99-3.88 (m, 1H), |
| | | | | 2.96 (s, 2H), 2.02 (d, J = 26.7 Hz, 3H), 1.81 (s, 1H), 1.59 (d, J = 9.6 Hz, 2H), |
| | | | | 1.46 (s, 9H). |
| 34 | C | 3.355 | 418.2 | 1H NMR (400 MHz, Methanol-d₄) d 8.15 (d, J = 4.0 Hz, 1H), 7.81-7.76 (m, 2H), |
| | | | | 7.68-7.63 (m, 2H), 7.61-7.55 (m, 1H), 7.09-7.05 (m, 2H), 3.41 (q, J = 7.2 Hz, |
| | | | | 2H), 1.24 (t, J = 3.6 Hz, 3H). |
| 35 | C | 2.773 | 471.2 | 1H NMR (400 MHz, Methanol-d₄) d 8.17 (s, 1H), 7.81-7.76 (m, 2H), 7.59 (td, J = |
| | | | | 8.3, 5.6 Hz, 1H), 7.50 (dt, J = 8.3, 1.4 Hz, 1H), 7.38 (td, J = 8.8, 1.4 Hz, 1H), |
| | | | | 7.08-7.02 (m, 2H), 4.02 (tt, J = 11.1, 4.1 Hz, 1H), 3.29-3.23 (m, 3H), 2.74 (t, |
| | | | | J = 12.3 Hz, 2H), 2.62 (d, J = 1.2 Hz, 3H), 2.08 (dd, J = 14.2, 3.8 Hz, 2H), 1.88- |
| | | | | 1.74 (m, 2H). |
| 36 | C | 3.013 | 457.2 | 1H NMR (400 MHz, Methanol-d₄) d 8.17 (s, 1H), 7.61 (td, J = 8.4, 5.6 Hz, 1H), |
| | | | | 7.52 (dt, J = 8.3, 1.3 Hz, 1H), 7.44-7.37 (m, 3H), 7.14-7.06 (m, 2H), 3.70 (s, |
| | | | | 4H), 2.55 (d, J = 4.9 Hz, 4H), 2.38 (s, 3H). |
| 37 | C | 3.915 | 557.2 | |
| 38 | C | 2.943 | 457.2 | 1H NMR (400 MHz, Methanol-d₄) d 8.20 (s, 1H), 7.81 (d, J = 8.8 Hz, 1H), 7.61 (td, |
| | | | | J = 8.3, 5.6 Hz, 1H), 7.52 (dt, J = 8.3, 1.4 Hz, 1H), 7.40 (ddd, J = 9.4, 8.6, |
| | | | | 1.3 Hz, 1H), 7.08 (d, J = 8.8 Hz, 2H), 4.12 (td, J = 10.1, 4.7 Hz, 1H), 3.29 (d, |
| | | | | J = 4.2 Hz, 1H), 3.12 (d, J = 12.8 Hz, 1H), 2.81-2.63 (m, 2H), 2.05 (d, J = 11.9 |
| | | | | Hz, 1H), 1.92 (s, 2H), 1.80-1.57 (m, 2H). |
| 39 | C | 3.050 | 473.2 | |
| 40 | C | 3.261 | 402.2 | |
| 41 | C | 4.256 | 431.2 | 1H NMR (400 MHz, Methanol-d₄) d 8.17 (s, 1H), 7.92-7.88 (m, 2H), 7.63 (dd, J = |
| | | | | 8.1, 1.1 Hz, 2H), 7.55 (dd, J = 9.2, 7.0 Hz, 1H), 7.06-7.00 (m, 2H), 1.37 (s, 9H). |
| 42 | C | 4.156 | 415.2 | 1H NMR (400 MHz, Methanol-d₄) d 8.21 (s, 1H), 7.91 (d, J = 8.8 Hz, 2H), 7.59 (td, |
| | | | | J = 8.4, 5.6 Hz, 1H), 7.50 (dt, J = 8.3, 1.4 Hz, 1H), 7.38 (ddd, J = 9.7, 8.5, |
| | | | | 1.4 Hz, 1H), 7.03 (d, J = 8.9 Hz, 1H), 1.37 (s, 9H). |
| 43 | C | 3.258 | 415.0 | 1H NMR (400 MHz, Methanol-d₄) d 8.01 (s, 1H), 7.66 (d, J = 1.4 Hz, 1H), 7.64 (d, |
| | | | | J = 0.7 Hz, 1H), 7.56 (dd, J = 9.2, 7.0 Hz, 1H), 7.37 (d, J = 8.7 Hz, 1H), 7.23 |
| | | | | (d, J = 2.2 Hz, 1H), 7.01 (dd, J = 8.6, 2.2 Hz, 1H), 3.77 (s, 3H), 2.57 (s, 3H). |
| 44 | C | 3.169 | 399.2 | 1H NMR (400 MHz, Methanol-d₄) d 8.04 (s, 1H), 7.60 (td, J = 8.4, 5.6 Hz, 1H), |
| | | | | 7.51 (dt, J = 8.3, 1.4 Hz, 1H), 7.43-7.34 (m, 2H), 7.23 (d, J = 2.2 Hz, 1H), 7.01 |
| | | | | (dd, J = 8.6, 2.1 Hz, 1H), 3.77 (s, 3H), 2.57 (s, 3H). |
| 45 | C | 3.473 | 440.3 | 1H NMR (400 MHz, Methanol-d₄) δ 8.94 (d, J = 5.1 Hz, 1H), 8.13 (s, 1H), 7.67-7.59 |
| | | | | (m, 3H), 7.59-7.50 (m, 3H), 7.22-7.15 (m, 2H), 2.43 (s, 3H). |
| 46 | C | 3.860 | 470.2 | 1H NMR (400 MHz, Methanol-d₄) d 8.15 (s, 1H), 7.67-7.61 (m, 4H), 7.57 (dd, J = |
| | | | | 9.2, 7.0 Hz, 1H), 7.12-6.99 (m, 2H), 4.39 (s, 2H), 4.12 (s, 2H), 2.25 (t, J = 7.6 |
| | | | | Hz, 4H), 1.94-1.79 (m, 2H). |
| 47 | C | 3.480 | 442.0 | 1H NMR (400 MHz, Methanol-d₄) d 8.18 (s, 1H), 7.98 (s, 1H), 7.66 (dd, J = 8.1, |
| | | | | 1.1 Hz, 2H), 7.61-7.57 (m, 3H), 7.24-7.19 (m, 2H), 4.32 (q, J = 7.2 Hz, 2H), 1.49 |
| | | | | (t, J = 7.2 Hz, 3H). |
| 48 | C | 3.450 | 428.0 | 1H NMR (400 MHz, Methanol-d₄) d 8.14 (s, 1H), 7.75 (s, 1H), 7.65 (dd, J = 8.1, |
| | | | | 1.1 Hz, 2H), 7.57 (dd, J = 9.2, 7.0 Hz, 1H), 7.48 (d, J = 8.7 Hz, 2H), 7.25-7.15 |
| | | | | (m, 2H), 4.11 (s, 3H). |
| 49 | C | 3.430 | 425.0 | 1H NMR (400 MHz, Methanol-d₄) d 9.05 (dd, J = 4.9, 1.5 Hz, 1H), 8.17 (s, 1H), |
| | | | | 8.12 (dd, J = 8.8, 1.5 Hz, 1H), 8.06 (d, J = 8.8 Hz, 2H), 7.73 (dd, J = 8.7, 4.9 |
| | | | | Hz, 1H), 7.66 (dd, J = 8.2, 1.1 Hz, 2H), 7.57 (dd, J = 9.2, 7.0 Hz, 1H), 7.21 (d, |
| | | | | J = 8.8 Hz, 2H). |
| 50 | C | 3.040 | 501.2 | 1H NMR (400 MHz, Methanol-d₄) d 8.13 (s, 1H), 7.65 (dd, J = 8.1, 1.1 Hz, 2H), |
| | | | | 7.57 (dd, J = 9.2, 7.0 Hz, 1H), 7.40 (d, J = 8.2 Hz, 2H), 7.12-7.07 (m, 2H), 3.33 |
| | | | | (s, 3H), 3.26 (bs, 1H), 2.98 (s, 3H), 2.60 (s, 4H), 2.09 (td, J = 12.9, 3.9 Hz, |
| | | | | 2H), 1.91 (d, J = 13.1 Hz, 2H). |
| 51 | C | 3.689 | 440.2 | 1H NMR (400 MHz, Methanol-d₄) δ 8.49-8.43 (m, 1H), 8.40 (d, J = 2.6 Hz, 1H), 8.12 |
| | | | | (s, 1H), 7.63 (dd, J = 8.1, 1.0 Hz, 2H), 7.59-7.53 (m, 3H), 7.20-7.13 (m, 2H), |
| | | | | 2.67-2.62 (m, 3H). |
| 52 | B | 3.739 | 454.2 | 1H NMR (400 MHz, Methanol-d₄) δ 8.96 (s, 1H), 8.68 (s, 1H), 8.14 (s, 1H), 7.67- |
| | | | | 7.60 (m, 2H), 7.60-7.51 (m, 3H), 7.20-7.13 (m, 2H), 2.85 (q, J = 7.6 Hz, 2H), 1.20 |
| | | | | (t, J = 7.6 Hz, 3H). |
| 53 | C | 3.858 | 441.2 | |
| 54 | C | 3.776 | 454.2 | 1H NMR (400 MHz, Methanol-d₄) d 8.18 (s, 1H), 7.66-7.56 (m, 3H), 7.52 (dt, J = |
| | | | | 8.3, 1.4 Hz, 1H), 7.40 (ddd, J = 9.7, 8.4, 1.4 Hz, 1H), 7.10-7.03 (m, 2H), 4.39 |
| | | | | (s, 2H), 4.12 (s, 2H), 2.25 (t, J = 7.6 Hz, 5H), 1.88 (td, J = 7.6, 4.8 Hz, 2H). |
| 55 | C | 3.317 | 468.2 | 1H NMR (400 MHz, Chloroform-d) d 8.49 (s, 1H), 7.69 (s, 1H), 7.53-7.46 (m, 2H), |
| | | | | 7.41 (dd, J = 9.0, 7.1 Hz, 1H), 7.10 (d, J = 6.3 Hz, 2H), 6.76 (s, 1H), 5.50 (s, |
| | | | | 1H), 4.14 (s, 2H), 3.15(s, 2H), 2.04(bs, 4H). |
| 56 | C | 3.385 | 423.2 | 1H NMR (400 MHz, Methanol-d₄) δ 8.95 (d, J = 5.2 Hz, 1H), 8.16 (s, 1H), 7.66-7.47 |
| | | | | (m, 5H), 7.38 (ddd, J = 9.6, 8.4, 1.4 Hz, 1H), 7.23-7.15 (m, 2H), 2.44 (s, 3H). |
| 57 | C | 3.146 | 471.2 | ¹H NMR (400 MHz, Methanol-d₄) δ 8.15 (s, 1H), 7.71-7.60 (m, 4H), 7.56 (dd, J = |
| | | | | 9.2, 7.0 Hz, 1H), 7.22-7.15 (m, 2H), 4.99 (s, 2H), 4.21 (dd, J = 6.0, 4.3 Hz, |
| | | | | 2H), 4.06 (dd, J = 6.0, 4.3 Hz, 2H). |
| 58 | C | 3.250 | 452.2 | 1H NMR (400 MHz, Methanol-d₄) d 8.19 (s, 1H), 7.66-7.57 (m, 3H), 7.52 (dt, J = |
| | | | | 8.3, 1.3 Hz, 1H), 7.40 (ddd, J = 9.6, 8.4, 1.4 Hz, 1H), 7.23-7.15 (m, 2H), 4.12 |
| | | | | (t, J = 5.6 Hz, 2H), 3.02 (t, J = 6.0 Hz, 2H), 2.02 (dt, J = 6.5, 3.6 Hz, 4H). |
| 59 | C | 3.855 | 449.0 | 1H NMR (400 MHz, Chloroform-d) d 8.87 (dd, J = 4.9, 1.8 Hz, 1H), 8.53 (s, 1H), |
| | | | | 8.08 (dd, J = 7.8, 1.8 Hz, 1H), 8.04-7.97 (m, 2H), 7.76 (s, 1H), 7.53 (dd, J = |
| | | | | 8.1, 0.9 Hz, 2H), 7.44 (dd, J = 9.1, 7.1 Hz, 1H), 7.34 (dd, J = 7.8, 4.9 Hz, 1H), |
| | | | | 7.21-7.14 (m, 2H). |
| 60 | C | 3.579 | 450.0 | |
| 61 | C | 3.834 | 488.2 | 1H NMR (400 MHz, Methanol-d₄) d 8.20 (s, 1H), 7.87-7.67 (m, 4H), 7.66-7.56 (m, |
| | | | | 2H), 7.11-6.98 (m, 2H), 4.38 (s, 2H), 4.12 (s, 2H), 2.24 (t, J = 7.6 Hz, 4H), |
| | | | | 1.87 (dq, J = 12.6, 7.4 Hz, 2H). |
| 62 | C | 3.065 | 454.2 | 1H NMR (400 MHz, Methanol-d₄) δ 8.18 (s, 1H), 7.71-7.64 (m, 2H), 7.59 (td, J = |
| | | | | 8.3, 5.6 Hz, 1H), 7.51 (dd, J = 8.1, 1.6 Hz, 1H), 7.38 (t, J = 8.5 Hz, 1H), 7.22- |
| | | | | 7.16 (m, 2H), 4.99 (s, 2H), 4.21 (dd, J = 6.0, 4.3 Hz, 2H), 4.07 (dd, J = 6.0, 4.3 Hz, 2H). |
| 63 | C | 3.450 | 483.4 | 1H NMR (400 MHz, Methanol-d₄) δ 8.14 (s, 1H), 7.63 (dd, J = 8.1, 1.1 Hz, 2H), |
| | | | | 7.56 (dd, J = 9.2, 7.0 Hz, 1H), 7.46 (d, J = 8.6 Hz, 2H), 7.22-7.15 (m, 2H), 4.15- |
| | | | | 4.08 (m, 2H), 3.08-3.00 (m, 2H), 1.98-1.90 (m, 2H), 1.83-1.76 (m, 4H). |
| 64 | C | 2.856 | 391.2 | |
| 65 | C | 4.298 | 463.2 | 1H NMR (400 MHz, Chloroform-d) d 8.27 (s, 1H), 8.16 (d, J = 1.0 Hz, 1H), 7.78 |
| | | | | (dt, J = 8.1, 1.0 Hz, 1H), 7.67 (s, 1H), 7.64 (dq, J = 8.6, 1.0 Hz, 1H), 7.61- |
| | | | | 7.56 (m, 2H), 7.49 (dd, J = 8.1, 0.9 Hz, 2H), 7.41-7.35 (m, 2H), 7.18-7.14 (m, |
| | | | | 2H), 6.75 (s, 1H), 5.42 (s, 1H). |
| 66 | C | 4.189 | 463.2 | |
| 67 | C | 3.688 | 460.2 | |
| 68 | C | 3.232 | 445.2 | |
| 69 | C | 3.806 | 494.2 | 1H NMR (400 MHz, Methanol-d₄) δ 9.42 (d, J = 5.3 Hz, 1H), 8.15 (s, 1H), 8.09 (d, |
| | | | | J = 5.4 Hz, 1H), 7.67-7.60 (m, 2H), 7.60-7.50 (m, 3H), 7.22-7.15 (m, 2H). |
| 70 | C | 3.656 | 469.2 | 1H NMR (400 MHz, Methanol-d₄) d 8.04 (d, J = 0.8 Hz, 1H), 7.85 (d, J = 0.7 Hz, |
| | | | | 1H), 7.64-7.59 (m, 2H), 7.54 (dd, J = 9.3, 6.9 Hz, 1H), 7.44 (s, 1H), 7.41-7.35 |
| | | | | (m, 2H), 7.18-7.12 (m, 2H), 4.73 (d, J = 1.3 Hz, 2H), 3.92 (t, J = 5.5 Hz, 2H), |
| | | | | 2.81 (dd, J = 6.2, 4.9 Hz, 2H). |
| 71 | C | 3.706 | 469.2 | 1H NMR (400 MHz, Methanol-d₄) d 7.99 (d, J = 0.9 Hz, 1H), 7.76 (s, 1H), 7.64-7.58 |
| | | | | (m, 2H), 7.56-7.50 (m, 3H), 7.15-7.07 (m, 2H), 4.75 (d, J = 0.9 Hz, 2H), 3.99 (t, |
| | | | | J = 5.7 Hz, 2H), 2.83 (t, J = 5.7 Hz, 2H). |
| 72 | C | 3.470 | 455.0 | 1H NMR (400 MHz, Methanol-d₄) d 8.11 (s, 1H), 7.67-7.62 (m, 2H), 7.56 (dd, J = |
| | | | | 9.2, 7.0 Hz, 1H), 7.48-7.40 (m, 3H), 7.17-7.11 (m, 2H), 7.01 (d, J = 9.5 Hz, 1H), |
| | | | | 2.40 (s, 3H). |
| 73 | C | 3.855 | 468.2 | 1H NMR (400 MHz, Methanol-d₄) d 8.01 (d, J = 3.0 Hz, 1H), 7.78 (d, J = 2.9 Hz, |
| | | | | 1H), 7.61 (d, J = 8.8 Hz, 2H), 7.54 (dd, J = 9.3, 6.8 Hz, 1H), 7.48-7.42 (m, 2H), |
| | | | | 7.22-7.16 (m, 2H), 2.82-2.70 (m, 4H), 1.90 (p, J = 3.7 Hz, 4H). |
| 74 | C | 3.326 | 461.2 | 1H NMR (400 MHz, Methanol-d₄) δ 8.61 (s, 1H), 8.36 (dd, J = 2.3, 0.8 Hz, 1H), |
| | | | | 7.68 (dd, J = 8.7, 2.3 Hz, 1H), 7.66-7.59 (m, 2H), 7.55 (dd, J = 9.2, 6.9 Hz, 1H), |
| | | | | 6.93 (dd, J = 8.7, 0.8 Hz, 1H), 3.91-3.49 (m, 8H). |
| 75 | C | 3.940 | 427.2 | 1H NMR (400 MHz, Methanol-d₄) d 8.07 (s, 1H), 7.93 (d, J = 2.4 Hz, 1H), 7.65 (dd, |
| | | | | J = 8.1, 1.1 Hz, 2H), 7.56 (dd, J = 9.2, 7.2 Hz, 3H), 7.18-7.11 (m, 2H), 6.28 (d, |
| | | | | J = 2.4 Hz, 1H), 2.34 (s, 3H). |
| 76 | C | 4.198 | 447.0 | 1H NMR (400 MHz, Methanol-d₄) d 8.09 (s, 1H), 8.08 (d, J = 2.5 Hz, 1H), 7.65 (dd, |
| | | | | J = 8.1, 1.1 Hz, 2H), 7.62-7.54 (m, 3H), 7.15 (d, J = 9.0 Hz, 2H), 6.44 (d, |
| | | | | J = 2.5 Hz, 1H). |
| 77 | C | 3.719 | 443.2 | 1H NMR (400 MHz, Methanol-d₄) d 7.96 (s, 1H), 7.63 (dd, J = 8.1, 1.1 Hz, 2H), |
| | | | | 7.55 (dd, J = 9.2, 7.0 Hz, 1H), 7.44 (d, J = 2.4 Hz, 1H), 5.69 (d, J = 2.4 Hz, |
| | | | | 1H), 3.77 (s, 3H). |
| 78 | C | 3.054 | 443.2 | |
| 79 | C | 3.845 | 448.0 | 1H NMR (400 MHz, Chloroform-d) d 8.42 (s, 1H), 8.35 (s, 1H), 7.68 (s, 1H), 7.58- |
| | | | | 7.50 (m, 4H), 7.45 (dd, J = 9.0, 7.1 Hz, 1H), 7.16-7.09 (m, 2H), 6.79 (s, 1H), |
| | | | | 5.44 (s, 1H). |
| 80 | C | 3.820 | 427.2 | 1H NMR (400 MHz, Methanol-d₄) d 8.12 (s, 1H), 7.68-7.62 (m, 2H), 7.57 (dd, J = |
| | | | | 9.2, 7.0 Hz, 1H), 7.52 (d, J = 1.8 Hz, 2H), 7.36-7.30 (m, 2H), 7.20-7.13 (m, 2H), |
| | | | | 6.25 (s, 1H), 2.30 (s, 3H). |
| 81 | C | 4.030 | 465.2 | 1H NMR (400 MHz, Chloroform-d) δ 8.44 (s, 1H), 8.11 (s, 1H), 7.72 (s, 1H), 7.72- |
| | | | | 7.62 (m, 3H), 7.53 (dd, J = 8.6, 7.3 Hz, 2H), 7.47-7.37 (m, 2H), 7.26-7.19 (m, |
| | | | | 2H), 6.79 (brs, 1H), 5.45 br (s, 1H). |
| 82 | A | 3.942 | 458.2 | 1H NMR (400 MHz, Methanol-d₄) δ 8.12 (s, 1H), 7.66 (d, J = 1.5 Hz, 1H), 7.64 (d, |
| | | | | J = 0.7 Hz, 1H), 7.59-7.56 (m, 1H), 7.39-7.35 (m, 2H), 7.11-7.06 (m, 2H), 3.61 |
| | | | | (m, J = 34.7 Hz, 4H), 1.73 (m, J = 5.8 Hz, 2H), 1.63 (m, 4H). |
| 83 | A | 3.428 | 523.2 | ¹H NMR (400 MHz, Methanol-d₄) δ 8.13 (s, 1H), 7.65 (dd, J = 8.1, 1.1 Hz, 2H), |
| | | | | 7.57 (dd, J = 9.2, 7.0 Hz, 1H), 7.45-7.37 (m, 2H), 7.14-7.06 (m, 2H), 6.25-5.89 |
| | | | | (m, 1H), 3.72 (m, J = 7.5 Hz, 4H), 2.99 (td, J = 15.1, 4.2 Hz, 2H), 2.81 (d, |
| | | | | J = 5.1 Hz, 4H). |
| 84 | C | 3.625 | 499.2 | ¹H NMR (400 MHz, Methanol-d₄) δ 8.13 (s, 1H), 7.65 (dd, J = 8.1, 1.1 Hz, 2H), |
| | | | | 7.57 (dd, J = 9.2, 7.0 Hz, 1H), 7.42-7.37 (m, 2H), 7.12-7.07 (m, 2H), 3.63 (m, |
| | | | | 4H), 2.68 (m, 4H), 1.73 (tt, J = 6.6, 3.7 Hz, 1H), 0.56-0.49 (m, J = 2.8 Hz, 2H), |
| | | | | 0.49-0.43 (m, 2H). |
| 85 | C | 3.414 | 560.2 | 1H NMR (400 MHz, Methanol-d₄) δ 8.20 (s, 1H), 7.70-7.61 (m, 4H), 7.56 (dd, J = |
| | | | | 9.2, 6.9 Hz, 1H), 7.19-7.13 (m, 2H), 3.01 (brs, 4H), 2.52 (brs, 4H), 2.27 (s, 3H). |
| 86 | A | 2.901 | 513.2 | ¹H NMR (600 MHz, Methanol-d₄) δ 8.10 (s, 1H), 7.61 (d, J = 8.1 Hz, 2H), 7.54 (dd, |
| | | | | J = 8.9, 7.4 Hz, 1H), 7.39-7.35 (m, 2H), 7.08-7.04 (m, 2H), 3.67 (m, 4H), 2.60 |
| | | | | (m, 4H), 2.32 (d, J = 6.7 Hz, 2H), 0.93-0.85 (m, 1H), 0.57-0.50 (m, 2H), 0.15 (q, |
| | | | | J = 5.1 Hz, 2H). |
| 87 | C | 3.234 | 442.2 | 1H NMR (400 MHz, Methanol-d₄) d 8.61 (s, 1H), 8.17 (s, 1H), 7.68-7.63 (m, 2H), |
| | | | | 7.62-7.56 (m, 3H), 7.24-7.19 (m, 2H), 4.19 (q, J = 7.3 Hz, 2H), 1.42 (t, J = 7.3 |
| | | | | Hz, 3H). |
| 88 | C | 3.103 | 428.2 | 1H NMR (400 MHz, Methanol-d₄) d 8.51 (s, 1H), 8.17 (d, J = 0.9 Hz, 1H), 7.68-7.61 |
| | | | | (m, 4H), 7.57 (ddd, J = 9.2, 7.0, 0.7 Hz, 1H), 7.24-7.18 (m, 2H), 3.82 (s, 3H). |
| 89 | C | 3.080 | 524.2 | 1H NMR (400 MHz, Methanol-d₄) δ 8.18 (s, 1H), 7.78-7.72 (m, 2H), 7.67-7.61 (m, |
| | | | | 2H), 7.56 (dd, J = 9.2, 6.9 Hz, 1H), 7.15-7.08 (m, 2H), 3.17-3.04 (m, 1H), 2.96- |
| | | | | 2.92 (m, 2H), 2.40-2.36 (m, 5H), 1.80-1.76 (m, 2H), 1.61-1.53 (m, 2H). |
| 90 | C | 3.669 | 458.2 | |
| 91 | C | 2.947 | 487.2 | ¹H NMR (400 MHz, Methanol-d₄) δ 8.07 (s, 1H), 7.63-7.58 (m, 2H), 7.53 (dd, J = |
| | | | | 9.2, 7.0 Hz, 1H), 7.38 (d, J = 8.2 Hz, 2H), 7.09-7.01 (m, 2H), 3.72 (m, J = 44.2 |
| | | | | Hz, 4H), 3.04 (m, 1H), 2.87 (s, 3H), 2.58 (m, 3H), 2.00 (m, 2H). |
| 92 | C | 3.363 | 430.2 | |
| 93 | C | 3.839 | 541.2 | |
| 94 | C | 3.526 | 444.2 | ¹H NMR (400 MHz, Methanol-d₄) δ 8.08 (s, 1H), 7.60 (dd, J = 8.1, 1.1 Hz, 2H), |
| | | | | 7.55-7.52 (m, 1H), 7.49 (dd, J = 8.4, 1.7 Hz, 2H), 7.07-7.00 (m, 2H), 3.55 (t, |
| | | | | J = 6.7 Hz, 4H), 1.95 (q, J = 6.6 Hz, 2H), 1.88 (q, J = 6.4 Hz, 2H). |
| 95 | C | 3.897 | 472.2 | ¹H NMR (400 MHz, Methanol-d₄) δ 8.06 (s, 1H), 7.62-7.58 (m, 2H), 7.52 (dd, J = |
| | | | | 9.2, 7.0 Hz, 1H), 7.36-7.26 (m, 2H), 7.09-7.01 (m, 2H), 3.62 (m, 2H), 3.50 (m, |
| | | | | 2H), 1.80 (m, 2H), 1.64 (m, 4H), 1.59 (m, 2H). |
| 96 | C | 3.325 | 430.2 | 1H NMR (400 MHz, Methanol-d₄) δ 8.15 (s, 1H), 7.94 (s, 1H), 7.77-7.60 (m, 4H), |
| | | | | 7.56 (dd, J = 9.2, 7.0 Hz, 1H), 7.31-6.96 (m, 2H), 3.99 (s, 3H). |
| 201 | C | 4.265 | 496.0 | 1H NMR (400 MHz, Methanol-d4) d 8.16 (s, 1H), 7.68-7.63 (m, 2H), 7.60-7.56 (m, |
| | | | | 2H), 7.34-7.28 (m, 3H), 7.28-7.22 (m, 3H), 6.85 (dd, J = 7.9, 1.2 Hz, 1H). |
| 202 | C | 3.573 | 482.2 | 1H NMR (400 MHz, Methanol-d4) d 8.70 (s, 1H), 8.15 (s, 1H), 7.68-7.59 (m, 2H), |
| | | | | 7.59-7.48 (m, 3H), 7.25-7.14 (m, 2H), 4.66 (d, J = 7.3 Hz, 1H), 2.30-2.12 (m, |
| | | | | 2H), 1.89 (d, J = 13.6 Hz, 4H), 1.79-1.64 (m, 2H). |
| 203 | C | 3.500 | 470.00 | 1H NMR (400 MHz, Methanol-d4) d 8.56 (s, 1H), 8.16 (s, 1H), 7.63 (dd, J = 8.1, |
| | | | | 1.1 Hz, 2H), 7.60-7.48 (m, 3H), 7.25-7.07 (m, 2H), 3.98 (d, J = 7.5 Hz, 2H), 1.95 |
| | | | | (dt, J = 13.7, 6.9 Hz, 1H), 0.81 (d, J = 6.7 Hz, 6H). |
| 204 | C | 3.38 | 427.2 | |
| 205 | C | 3.354 | 456.00 | 1H NMR (400 MHz, Methanol-d₄) δ 8.73 (s, 1H), 8.13 (s, 1H), 7.61 (dd, J = 8.1, |
| | | | | 1.0 Hz, 2H), 7.53 (dd, J = 9.2, 7.0 Hz, 1H), 7.50-7.45 (m, 2H), 7.20-7.15 (m, |
| | | | | 2H), 4.54 (h, J = 6.7 Hz, 1H), 1.48 (d, J = 6.7 Hz, 6H). |
| 206 | C | 3.295 | 454.00 | 1H NMR (400 MHz, Methanol-d₄) δ 8.56 (s, 1H), 8.19 (s, 1H), 7.85-7.79 (m, 2H), |
| | | | | 7.66 (dd, J = 8.1, 1.1 Hz, 2H), 7.58 (dd, J = 9.2, 7.0 Hz, 1H), 7.23-7.16 (m, |
| | | | | 2H), 3.61 (tt, J = 7.3, 3.8 Hz, 1H), 1.18-1.10 (m, 2H), 1.00-0.93 (m, 2H). |
| 207 | C | 3.14 | 426.2 | 1H NMR (400 MHz, Methanol-d₄) δ 8.60 (s, 1H), 8.18 (s, 1H), 7.66-7.47 (m, 4H), |
| | | | | 7.38 (ddd, J = 9.6, 8.4, 1.4 Hz, 1H), 7.26-7.13 (m, 2H), 4.18 (q, J = 7.3 Hz, |
| | | | | 2H), 1.40 (t, J = 7.3 Hz, 3H). |
| 208 | C | 3.56 | 453.2 | 1H NMR (400 MHz, Methanol-d₄) δ 8.12 (s, 1H), 7.58 (td, J = 8.3, 5.6 Hz, 1H), |
| | | | | 7.50 (dt, J = 8.3, 1.3 Hz, 1H), 7.47-7.31 (m, 4H), 7.21-7.09 (m, 2H), 4.72 (2, |
| | | | | 2H), 3.91 (t, J = 5.7 Hz, 2H), 2.81 (t, J = 5.7 Hz, 2H). |
| 209 | C | 3.61 | 453.2 | 1H NMR (400 MHz, Methanol-d₄) δ 8.09 (s, 1H), 7.79 (s, 1H), 7.62-7.47 (m, 4H), |
| | | | | 7.42-7.33 (m, 1H), 7.15-7.07 (m, 2H), 4.75 (s, 2H), 3.99 (t, J = 5.7 Hz, 2H), |
| | | | | 2.83 (t, J = 5.7 Hz, 2H). |
| 210 | C | 3.400 | 496.00 | 1H NMR (400 MHz, Methanol-d4) d 8.71 (s, 1H), 8.19 (s, 1H), 7.65 (dd, J = 8.1, |
| | | | | 1.1 Hz, 2H), 7.60-7.52 (m, 3H), 7.27-7.19 (m, 2H), 5.00 (q, J = 8.6 Hz, 2H). |
| 211 | C | 3.570 | 440.20 | 1H NMR (400 MHz, Methanol-d4) d 8.20 (s, 1H), 7.99 (d, J = 0.7 Hz, 1H), 7.61 (td, |
| | | | | J = 8.3, 5.6 Hz, 1H), 7.56-7.49 (m, 3H), 7.40 (td, J = 8.8, 8.4, 1.4 Hz, 1H), |
| | | | | 7.25-7.18 (m, 2H), 4.80 (p, J = 6.7 Hz, 1H), 1.51 (d, J = 6.6 Hz, 6H). |
| 212 | C | 3.240 | 478.00 | 1H NMR (400 MHz, Methanol-d4) d 8.63 (d, J = 1.1 Hz, 1H), 8.18 (s, 1H), 7.65 (dd, |
| | | | | J = 8.1, 1.1 Hz, 2H), 7.60-7.52 (m, 3H), 7.25-7.18 (m, 2H), 6.22 (tt, J = 54.3, |
| | | | | 2.9 Hz, 1H), 4.60 (td, J = 15.5, 2.9 Hz, 2H). |
| 213 | C | 3.590 | 453.20 | 1H NMR (400 MHz, Methanol-d4) d 8.88 (s, 1H), 8.14 (s, 1H), 7.68-7.63 (m, 2H), |
| | | | | 7.57 (dd, J = 9.2, 7.0 Hz, 1H), 7.51-7.46 (m, 2H), 7.23-7.17 (m, 2H), 2.43 (s, |
| | | | | 3H), 2.36 (s, 3H). |
| 214 | C | 2.94 | 485.2 | 1H NMR (400 MHz, Methanol-d₄) δ 8.16 (s, 1H), 7.71-7.50 (m, 6H), 7.20 (d, J = 8.7 |
| | | | | Hz, 2H), 4.29 (t, J = 6.6 Hz, 2H), 2.77 (t, J = 6.6 Hz, 2H), 2.30 (s, 6H). |
| 215 | C | 4.070 | 496.00 | 1H NMR (400 MHz, Methanol-d4) d 8.19 (s, 1H), 7.76-7.68 (m, 2H), 7.66 (dd, J = |
| | | | | 8.1, 1.1 Hz, 2H), 7.58 (dd, J = 9.2, 7.0 Hz, 1H), 7.26-7.18 (m, 2H), 4.08 (s, 3H). |
| 216 | C | 3.846 | 441.20 | 1H NMR (400 MHz, Methanol-d4) d 8.11 (s, 1H), 7.65 (dd, J = 8.1, 1.1 Hz, 2H), |
| | | | | 7.57 (dd, J = 9.2, 7.0 Hz, 1H), 7.34-7.28 (m, 2H), 7.19-7.13 (m, 2H), 6.03 (s, |
| | | | | 1H), 2.31-2.15 (m, 6H). |
| 217 | C | 4.120 | 441.20 | 1H NMR (400 MHz, Methanol-d4) d 8.06 (s, 1H), 7.76 (s, 1H), 7.65 (dd, J = 8.1, |
| | | | | 1.0 Hz, 2H), 7.56 (dd, J = 9.2, 7.0 Hz, 1H), 7.54-7.49 (m, 2H), 7.16-7.09 (m, |
| | | | | 2H), 2.26 (s, 3H), 2.09 (d, J = 0.9 Hz, 3H). |
| 218 | C | 3.997 | 441.20 | 1H NMR (400 MHz, Methanol-d4) d 8.12 (s, 1H), 7.65 (dd, J = 8.1, 1.1 Hz, 2H), |
| | | | | 7.57 (dd, J = 9.2, 7.0 Hz, 1H), 7.39 (s, 1H), 7.35-7.27 (m, 2H), 7.19-7.12 (m, |
| | | | | 2H), 2.20 (s, 3H), 2.08 (s, 3H). |
| 219 | C | 4.384 | 475.00 | 1H NMR (400 MHz, Methanol-d4) d 8.13 (s, 1H), 7.65 (dd, J = 8.1, 1.1 Hz, 2H), |
| | | | | 7.57 (dd, J = 9.2, 7.0 Hz, 1H), 7.35-7.30 (m, 2H), 7.20-7.14 (m, 2H), 2.25 (s, |
| | | | | 3H), 2.24 (s, 3H). |
| 220 | C | 4.421 | 461.20 | 1H NMR (400 MHz, Methanol-d4) d 8.12 (s, 1H), 8.08 (s, 1H), 7.65 (dd, J = 8.1, |
| | | | | 1.1 Hz, 2H), 7.57 (dt, J = 9.1, 3.6 Hz, 3H), 7.18-7.11 (m, 2H), 2.30 (s, 3H). |
| 221 | C | 4.243 | 461.20 | 1H NMR (400 MHz, Methanol-d4) d 8.13 (s, 1H), 7.65 (dd, J = 8.1, 1.1 Hz, 2H), |
| | | | | 7.60-7.56 (m, 2H), 7.37-7.32 (m, 2H), 7.21-7.15 (m, 2H), 2.27 (s, 3H). |
| 222 | C | 4.274 | 469.20 | 1H NMR (600 MHz, Methanol-d4) d 8.08 (s, 1H), 7.64-7.60 (m, 2H), 7.54 (dd, J = |
| | | | | 8.9, 7.4 Hz, 1H), 7.28-7.24 (m, 2H), 7.15-7.10 (m, 2H), 2.44 (q, J = 7.6 Hz, 2H), |
| | | | | 2.20 (s, 3H), 2.16 (s, 3H), 1.11 (t, J = 7.5 Hz, 3H). |
| 223 | C | 4.329 | 469.20 | 1H NMR (600 MHz, Methanol-d4) d 8.10 (s, 1H), 7.64-7.59 (m, 2H), 7.54 (dd, J = |
| | | | | 8.9, 7.4 Hz, 1H), 7.29-7.23 (m, 2H), 7.16-7.10 (m, 2H), 6.08 (s, 1H), 2.60 (dq, |
| | | | | J = 20.4, 7.6 Hz, 4H), 1.24 (t, J = 7.6 Hz, 3H), 1.16 (t, J = 7.5 Hz, 3H). |
| 224 | C | 4.105 | 455.20 | 1H NMR (600 MHz, Methanol-d4) d 8.08 (s, 1H), 7.65-7.58 (m, 2H), 7.54 (dd, J = |
| | | | | 8.9, 7.5 Hz, 1H), 7.29-7.23 (m, 2H), 7.17-7.08 (m, 2H), 2.17 (s, 3H), 2.14 (s, |
| | | | | 3H), 1.97 (s, 3H). |
| 225 | C | 3.09 | 427.2 | 1H NMR (400 MHz, Methanol-d₄) δ 8.11 (s, 1H), 7.67-7.47 (m, 5H), 7.20-7.08 (m, |
| | | | | 3H), 6.99 (d, J = 1.4 Hz, 1H), 3.74 (s, 3H). |
| 226 | C | 3.547 | 445.20 | 1H NMR (400 MHz, Methanol-d4) d 8.63 (s, 1H), 8.50 (dd, J = 2.4, 0.8 Hz, 1H), |
| | | | | 7.81 (dd, J = 8.7, 2.3 Hz, 1H), 7.65 (dd, J = 8.1, 1.1 Hz, 2H), 7.57 (dd, J = 9.2, |
| | | | | 6.9 Hz, 1H), 6.93 (dd, J = 8.7, 0.8 Hz, 1H), 3.62 (q, J = 7.2 Hz, 4H), 2.00 (td, |
| | | | | J = 12.1, 11.6, 6.1 Hz, 4H). |
| 227 | C | 3.43 | 428.2 | 1H NMR (400 MHz, Methanol-d₄) δ 8.14 (s, 1H), 7.66-7.43 (m, 4H), 7.37 (ddd, J = |
| | | | | 9.6, 8.4, 1.4 Hz, 1H), 7.13-6.99 (m, 2H), 3.57 (t, J = 6.7 Hz, 4H), 2.00-1.88 (m, 4H). |
| 228 | C | 3.68 | 442.2 | 1H NMR (400 MHz, Methanol-d₄) δ 8.13 (s, 1H), 7.57 (dd, J = 8.3, 5.6 Hz, 1H), |
| | | | | 7.50 (dt, J = 8.3, 1.4 Hz, 1H), 7.43-7.27 (m, 3H), 7.13-6.96 (m, 2H), 3.58 (brs, |
| | | | | 4H), 1.66 (brs, 6H). |
| 229 | C | 3.442 | 441.20 | 1H NMR (400 MHz, Methanol-d4) d 8.13 (s, 1H), 7.65 (dd, J = 8.1, 1.0 Hz, 2H), |
| | | | | 7.57 (dd, J = 9.2, 7.0 Hz, 1H), 7.52-7.47 (m, 2H), 7.24 (d, J = 1.5 Hz, 1H), |
| | | | | 7.20-7.14 (m, 2H), 7.06 (d, J = 1.5 Hz, 1H), 4.11 (q, J = 7.3 Hz, 2H), 1.40 (t, J = |
| | | | | 7.3 Hz, 3H). |
| 230 | C | 3.525 | 477.20 | 1H NMR (400 MHz, Methanol-d4) d 8.14 (s, 1H), 7.65 (dd, J = 8.1, 1.1 Hz, 2H), |
| | | | | 7.57 (dd, J = 9.2, 7.0 Hz, 1H), 7.51-7.45 (m, 2H), 7.29 (d, J = 1.4 Hz, 1H), 7.21- |
| | | | | 7.14 (m, 2H), 7.10 (d, J = 1.5Hz, 1H), 6.16 (tt, J = 54.8, 3.4 Hz, 1H), 4.47 (td, |
| | | | | J = 15.0, 3.4 Hz, 2H). |
| 231 | B | 3.96 | 479.2 | 1H NMR (400 MHz, Methanol-d₄) δ 8.20 (d, J = 0.5 Hz, 1H), 7.71 (d, J = 8.8 Hz, |
| | | | | 2H), 7.59 (td, J = 8.3, 5.6 Hz, 1H), 7.54-7.48 (m, 1H), 7.38 (ddd, J = 9.2, 8.4, |
| | | | | 1.4 Hz, 1H), 7.20 (d, J = 8.9 Hz, 2H), 4.06 (s, 3H). |
| 232 | B | 3.8 | 410.2 | 1H NMR (400 MHz, Methanol-d₄) δ 8.14 (s, 1H), 7.58 (td, J = 8.4, 5.6 Hz, 1H), |
| | | | | 7.53-7.46 (m, 2H), 7.37 (ddd, J = 9.6, 8.4, 1.4 Hz, 1H), 7.32 (d, J = 8.8 Hz, |
| | | | | 2H), 7.15 (d, J = 8.8 Hz, 2H), 6.23 (dd, J = 1.9, 0.9 Hz, 1H), 2.28 (d, J = 0.6 |
| | | | | Hz, 3H). |
| 233 | B | 3.69 | 410.2 | 1H NMR (400 MHz, Methanol-d₄) δ 8.14 (d, J = 0.5 Hz, 1H), 7.58 (td, J = 8.3, 5.6 |
| | | | | Hz, 1H), 7.53-7.46 (m, 2H), 7.42-7.33 (m, 1H), 7.32 (d, J = 8.9 Hz, 1H), 7.16 (d, |
| | | | | J = 8.9 Hz, 2H), 6.23 (dd, J = 1.9, 0.8 Hz, 1H), 2.28 (t, J = 0.6 Hz, 3H). |
| 234 | C | 4.11 | 452.2 | |
| 235 | C | 3.45 | 446.2 | |
| 236 | C | 4.78 | 535.2 | 1H NMR (400 MHz, Methanol-d₄) δ 8.11 (s, 1H), 7.69-7.48 (m, 3H), 7.45-7.34 (m, |
| | | | | 2H), 7.23-7.00 (m, 2H), 2.71-2.61 (m, 4H), 1.84-1.81 (m, 4H). |
| 237 | C | 3.165 | 460.20 | 1H NMR (400 MHz, Methanol-d4) d 7.87 (s, 1H), 7.63 (dd, J = 8.1, 1.1 Hz, 2H), |
| | | | | 7.55 (dd, J = 9.2, 7.0 Hz, 1H), 7.05-6.99 (m, 2H), 6.99-6.93 (m, 2H), 4.47 (s, |
| | | | | 1H), 3.24-3.13 (m, 2H), 2.97 (s, 2H), 2.70 (s, 3H), 2.15-1.93 (m, 4H). |
| 238 | C | 3.436 | 429.20 | 1H NMR (400 MHz, Methanol-d4) d 8.21 (s, 1H), 7.81-7.73 (m, 2H), 7.66 (dd, J = |
| | | | | 8.1, 1.1 Hz, 2H), 7.58 (dd, J = 9.2, 7.0 Hz, 1H), 7.29-7.19 (m, 2H), 4.22 (s, 3H). |
| 239 | C | 4.53 | 441.2 | 1H NMR (400 MHz, Methanol-d₄) δ 8.04 (s, 1H), 7.70-7.46 (m, 7H), 7.17-6.99 (m, 4H). |
| 240 | C | 4.181 | 441.20 | 1H NMR (400 MHz, Methanol-d4) d 8.07 (s, 1H), 7.94 (d, J = 2.4 Hz, 1H), 7.65 (dd, |
| | | | | J = 8.1, 1.1 Hz, 2H), 7.60-7.53 (m, 3H), 7.18-7.11 (m, 2H), 6.32 (d, J = 2.4 Hz, |
| | | | | 1H), 2.72 (q, J = 7.6 Hz, 2H), 1.30 (t, J = 7.6 Hz, 3H). |
| 241 | C | 4.012 | 441.20 | 1H NMR (400 MHz, Methanol-d4) d 8.13 (s, 1H), 7.65 (dd, J = 8.1, 1.1 Hz, 2H), |
| | | | | 7.61-7.55 (m, 1H), 7.55-7.52 (m, 1H), 7.36-7.29 (m, 2H), 7.21-7.14 (m, 2H), 6.28 |
| | | | | (dd, J = 1.9, 0.9 Hz, 1H), 2.66 (q, J = 7.6 Hz, 2H), 1.21 (t, J = 7.5 Hz, 3H). |
| 242 | C | 3.67 | 96.2 | |
| 243 | C | 3.472 | 429.20 | 1H NMR (400 MHz, Methanol-d4) d 8.19 (s, 1H), 7.66 (dd, J = 8.1, 1.1 Hz, 2H), |
| | | | | 7.58 (dd, J = 9.2, 7.0 Hz, 1H), 7.50-7.44 (m, 2H), 7.28-7.22 (m, 2H), 2.59 (s, 3H). |
| 244 | C | 4.551 | 441.20 | 1H NMR (400 MHz, Methanol-d4) d 8.07 (s, 1H), 7.65 (dd, J = 8.1, 1.1 Hz, 2H), |
| | | | | 7.62-7.56 (m, 3H), 7.44-7.39 (m, 2H), 7.32 (dt, J = 10.7, 1.9 Hz, 1H), 7.16-7.10 |
| | | | | (m, 2H), 7.03-6.96 (m, 1H). |
| 245 | C | 4.549 | 459.00 | 1H NMR (400 MHz, Methanol-d4) d 8.09 (s, 1H), 7.67-7.62 (m, 2H), 7.60-7.56 (m, |
| | | | | 1H), 7.54-7.49 (m, 2H), 7.23 (td, J = 6.2, 3.1 Hz, 1H), 7.15 (d, J = 1.0 Hz, 1H), |
| | | | | 7.15-7.11 (m, 2H), 7.02 (ddt, J = 9.0, 7.6, 3.4 Hz, 1H). |
| 246 | A | 3.619 | 462.2 | 1H NMR (600 MHz, cd3od) δ 8.12 (s, 1H), 7.64-7.60 (m, 2H), 7.57-7.50 (m, 3H), |
| | | | | 7.09-7.05 (m, 2H), 5.28 (t, J = 55.1 Hz, 1H), 3.94-3.65 (m, 4H), 2.37-2.05 (m, 2H). |
| 247 | A | 3.835 | 481.2 | 1H NMR (600 MHz, cd3od) δ 8.12 (s, 1H), 7.65-7.60 (m, 2H), 7.58-7.50 (m, 3H), |
| | | | | 7.09-7.05 (m, 2H), 3.94 (t, J = 12.6 Hz, 2H), 3.88-3.78 (m, 2H), 2.43 (s, 2H). |
| 248 | A | 2.774 | 487.2 | 1H NMR (600 MHz, cd3od) δ 8.11 (s, 1H), 7.65-7.60 (m, 2H), 7.57-7.48 (m, 3H), |
| | | | | 7.08-7.03 (m, 2H), 3.89-3.53 (m, 4H), 3.48-3.38 (m, 1H), 2.86 (d, J = 72.7 Hz, |
| | | | | 1H), 2.34 (s, 3H), 2.25 (s, 3H), 1.84 (dq, J = 28.9, 9.8 Hz, 1H). |
| 249 | A | 2.822 | 513.2 | 1H NMR (600 MHz, cd3od) δ 8.11 (s, 1H), 7.62 (d, J = 8.0 Hz, 2H), 7.57-7.49 (m, |
| | | | | 3H), 7.06 (d, J = 7.5 Hz, 2H), 3.86-3.55 (m, 3H), 3.48 (dd, J = 11.4, 8.0 Hz, 1H), |
| | | | | 2.90 (d, J = 76.4 Hz, 1H), 2.61 (d, J = 83.4 Hz, 4H), 2.18 (d, J = 50.1 Hz, 1H), |
| | | | | 1.83 (d, J = 38.4 Hz, 5H). |
| 250 | C | 4.36 | 455.2 | 1H NMR (400 MHz, Chloroform-d) δ 8.16 (s, 1H), 7.61 (s, 1H), 7.56-7.47 (m, 4H), |
| | | | | 7.41 (dd, J = 9.0, 7.1 Hz, 1H), 7.11-7.03 (m, 2H), 2.71 (q, J = 7.6 Hz, 2H), 2.09 |
| | | | | (s, 3H), 1.29 (t, J = 7.6 Hz, 7H). |
| 251 | C | 3.075 | 484.20 | 1H NMR (400 MHz, Methanol-d4) d 8.09 (s, 1H), 7.63-7.59 (m, 2H), 7.53 (dd, J = |
| | | | | 9.2, 7.0 Hz, 1H), 7.46 (d, J = 8.2 Hz, 2H), 7.22 (s, 1H), 7.13 (d, J = 8.1 Hz, |
| | | | | 2H), 6.99 (s, 1H), 4.15 (t, J = 7.1 Hz, 2H), 2.61 (t, J = 7.1 Hz, 2H), 2.16 (s, 6H). |
| 252 | C | 4.028 | 452.20 | 1H NMR (400 MHz, Chloroform-d) d 8.34 (s, 1H), 8.08 (s, 1H), 7.64 (s, 1H), 7.53- |
| | | | | 7.49 (m, 4H), 7.42 (dd, J = 9.0, 7.1 Hz, 1H), 7.12-7.06 (m, 2H), 6.76 (s, 1H), |
| | | | | 5.41 (s, 1H), 2.47 (s, 3H). |
| 253 | C | 3.867 | 452.20 | 1H NMR (400 MHz, Chloroform-d) d 8.42 (s, 1H), 7.83 (s, 1H), 7.67 (s, 1H), 7.51 |
| | | | | (dd, J = 8.1, 1.0 Hz, 2H), 7.42 (dd, J = 9.1, 7.1 Hz, 1H), 7.32-7.27 (m, 2H), |
| | | | | 7.13-7.07 (m, 2H), 6.77 (s, 1H), 5.44 (s, 1H), 2.44 (s, 3H). |
| 254 | C | 3.959 | 516.2 | 1H NMR (400 MHz, cd3od) δ 8.11 (s, 1H), 7.61 (dd, J = 8.1, 1.1 Hz, 2H), 7.57-7.49 |
| | | | | (m, 3H), 7.10-7.04 (m, 2H), 4.18 (t, J = 13.6 Hz, 4H). |
| 255 | C | 4.51 | 441.2 | 1H NMR (600 MHz, Methanol-d₄) δ 8.05 (s, 1H), 7.66-7.60 (m, 2H), 7.55 (dd, J = |
| | | | | 8.9, 7.5 Hz, 1H), 7.51-7.43 (m, 3H), 7.31-7.24 (m, 1H), 7.22-7.17 (m, 1H), 7.16- |
| | | | | 7.07 (m, 3H). |
| 256 | C | 4.27 | 448.2 | 1H NMR (600 MHz, Methanol-d₄) δ 8.10 (s, 1H), 7.78 (dd, J = 7.9, 1.3 Hz, 1H), |
| | | | | 7.68 (td, J = 7.7, 1.4 Hz, 1H), 7.65-7.61 (m, 2H), 7.58-7.54 (m, 2H), 7.54-7.50 |
| | | | | (m, 2H), 7.45 (td, J = 7.6, 1.2 Hz, 1H), 7.17-7.13 (m, 2H). |
| 257 | C | 4.38 | 495.2 | 1H NMR (600 MHz, Chloroform-d) δ 8.36 (brs, 1H), 7.66 (s, 1H), 7.52-7.48 (m, 2H), |
| | | | | 7.42 (dd, J = 8.8, 7.5 Hz, 1H), 7.34-7.30 (m, 2H), 7.12-7.07 (m, 2H), 6.77 (brs, |
| | | | | 1H), 6.42 (s, 1H), 5.46 (brs, 1H), 2.30 (s, 3H). |
| 258 | C | 4.35 | 495.2 | 1H NMR (600 MHz, Chloroform-d) δ 8.36 (brs, 1H), 7.66 (s, 1H), 7.52-7.48 (m, 2H), |
| | | | | 7.41 (dd, J = 8.8, 7.5 Hz, 1H), 7.37-7.32 (m, 2H), 7.08-7.04 (m, 2H), 6.75 (brs, |
| | | | | 1H), 6.55 (s, 1H), 5.39 (brs, 1 H), 2.35 (s, 3H). |
| 259 | C | 3.629 | 443.20 | 1H NMR (600 MHz, Methanol-d4) d 8.17 (s, 1H), 7.65-7.61 (m, 2H), 7.55 (dd, J = |
| | | | | 8.9, 7.4 Hz, 1H), 7.43-7.40 (m, 2H), 7.25-7.19 (m, 2H), 2.91 (q, J = 7.6 Hz, 2H), |
| | | | | 1.32 (t, J = 7.6 Hz, 3H). |
| 260 | C | 3.91 | 438.2 | 1H NMR (400 MHz, Methanol-d₄) δ 8.31 (t, J = 2.6 Hz, 2H), 8.07 (s, 1H), 7.63 (dd, |
| | | | | J = 8.1, 1.0 Hz, 2H), 7.55 (dd, J = 9.2, 7.0 Hz, 1H), 7.33 (d, J = 5.1 Hz, 1H), |
| | | | | 7.31-7.24 (m, 2H), 7.18-7.09 (m, 2H), 2.35 (s, 3H). |
| 261 | C | 4.226 | 405.20 | 1H NMR (400 MHz, Methanol-d4) d 7.80 (s, 1H), 7.60-7.56 (m, 2H), 7.50 (dd, J = |
| | | | | 9.2, 7.0 Hz, 1H), 6.97-6.93 (m, 2H), 6.85-6.81 (m, 2H), 4.43 (p, J = 6.1 Hz, 1H), |
| | | | | 1.26 (s, 3H), 1.24 (s, 3H). |
| 262 | C | 4.403 | 455.20 | 1H NMR (400 MHz, Chloroform-d) d 8.16 (s, 1H), 7.69 (d, J = 2.4 Hz, 1H), 7.60 (s, |
| | | | | 1H), 7.57-7.51 (m, 2H), 7.51-7.45 (m, 2H), 7.39 (dd, J = 9.0, 7.1 Hz, 1H), 7.09- |
| | | | | 7.03 (m, 2H), 6.72 (s, 1H), 6.24 (d, J = 2.4 Hz, 1H), 5.36 (s, 1H), 3.10 (h, J = |
| | | | | 6.9 Hz, 1H), 1.31 (s, 3H), 1.30 (s, 3H). |
| 263 | C | 4.149 | 455.20 | |
| 264 | C | 3.54 | 474.2 | |
| 265 | C | 3.28 | 426.2 | |
| 266 | C | 3.753 | 372.20 | 1H NMR (400 MHz, Methanol-d4) d 8.15 (s, 1H), 7.61 (dd, J = 8.1, 1.1 Hz, 2H), |
| | | | | 7.58-7.52 (m, 3H), 7.11-7.05 (m, 2H). |
| 267 | C | 3.305 | 354.20 | 1H NMR (400 MHz, Methanol-d4) d 8.24 (t, J = 1.4 Hz, 1H), 8.16 (dd, J = 6.7, 2.0 |
| | | | | Hz, 1H), 8.07 (s, 1H), 7.63-7.58 (m, 2H), 7.52 (dd, J = 9.2, 7.0 Hz, 1H), 7.39 |
| | | | | (d, J = 6.6 Hz, 1H), 7.36-7.32 (m, 2H), 7.15-7.10(m, 2H), 2.34 (s, 3H). |
| 268 | C | 3.710 | 495.20 | |
| 269 | C | 3.1 | 483.2 | 1H NMR (400 MHz, Methanol-d₄) δ 8.15 (s, 1H), 7.70-7.52 (m, 5H), 7.24-7.11 (m, |
| | | | | 2H), 4.20 (t, J = 5.5 Hz, 2H), 3.87 (s, 2H), 2.96 (t, J = 5.5 Hz, 2H), 2.57 (s, 2H). |
| 270 | C | 3.48 | 454.2 | 1H NMR (400 MHz, Methanol-d₄) δ 7.97 (s, 1H), 7.62 (dd, J = 8.1, 1.1 Hz, 2H), |
| | | | | 7.54 (dd, J = 9.2, 7.0 Hz, 1H), 7.23-7.15 (m, 2H), 7.03-6.97 (m, 2H), 3.01 (q, J = |
| | | | | 7.4 Hz, 2H), 1.31 (t, J = 7.4 Hz, 3H). |
| 271 | C | 3.35 | 440.2 | |
| 272 | C | 3.505 | 466.20 | 1H NMR (400 MHz, Methanol-d4) d 8.01 (s, 1H), 7.64 (dd, J = 8.1, 1.1 Hz, 2H), |
| | | | | 7.56 (dd, J = 9.2, 7.0 Hz, 1H), 7.29-7.22 (m, 2H), 7.10-7.02 (m, 2H), 3.74 (t, |
| | | | | J = 6.6 Hz, 2H), 3.40 (dd, J = 8.2, 7.0Hz, 2H), 2.49 (dq, J = 8.3, 6.7 Hz, 2H). |
| 273 | C | 2.999 | 486.20 | 1H NMR (400 MHz, Methanol-d4) d 8.20 (s, 1H), 7.68-7.63 (m, 2H), 7.58 (dd, J = |
| | | | | 9.2, 7.0 Hz, 1H), 7.49-7.44 (m, 2H), 7.28-7.23 (m, 2H), 3.13 (t, J = 7.3 Hz, 2H), |
| | | | | 2.82 (t, J = 7.4 Hz, 2H), 2.27 (s, 6H). |
| 274 | C | 4.37 | 524.2 | 1H NMR (600 MHz, Methanol-d₄) δ 8.16 (s, 1H), 7.67-7.60 (m, 2H), 7.60-7.51 (m, |
| | | | | 1H), 7.43-7.35 (m, 2H), 7.26-7.12 (m, 2H), 3.17 (sept, J = 6.9 Hz, 1H), 1.29 (d, |
| | | | | J = 6.9 Hz, 5H). |
| 275 | C | 4.05 | 496.2 | 1H NMR (600 MHz, Methanol-d₄) δ 8.15 (s, 1H), 7.69-7.60 (m, 2H), 7.56 (dd, J = |
| | | | | 8.9, 7.5 Hz, 1H), 7.49-7.40 (m, 2H), 7.28-7.11 (m, 2H), 2.51 (s, 3H). |
| 276 | C | 3.32 | 455.2 | 1H NMR (600 MHz, Methanol-d₄) δ 8.14 (s, 1H), 7.68-7.51 (m, 5H), 7.10-7.00 (m, |
| | | | | 2H), 4.74-4.17 (m, 4H), 3.81-3.73 (m, 1H). |
| 277 | C | 4.230 | 510.20 | 1H NMR (400 MHz, Methanol-d4) d 8.17 (s, 1H), 7.65 (dd, J = 8.0, 1.1 Hz, 2H), |
| | | | | 7.57 (dd, J = 9.2, 7.0 Hz, 1H), 7.45-7.39 (m, 2H), 7.25-7.18 (m, 2H), 2.85 (q, |
| | | | | J = 7.6 Hz, 2H), 1.31 (t, J = 7.6 Hz, 3H). |
| 278 | C | 4.245 | 510.20 | 1H NMR (400 MHz, Methanol-d4) d 8.19 (s, 1H), 7.68-7.62 (m, 4H), 7.58 (dd, J = |
| | | | | 9.2, 7.0 Hz, 1H), 7.25-7.20 (m, 2H), 4.39 (q, J = 7.2 Hz, 2H), 1.53 (t, J = 7.2 |
| | | | | Hz, 3H). |
| 279 | C | 4.388 | 524.20 | 1H NMR (400 MHz, Methanol-d4) d 8.18 (s, 1H), 7.67-7.63 (m, 2H), 7.57 (dd, J = |
| | | | | 9.2, 7.0 Hz, 1H), 7.44-7.38 (m, 2H), 7.24-7.19 (m, 2H), 2.81 (dd, J = 8.0, 7.1 |
| | | | | Hz, 2H), 1.77 (h, J = 7.4 Hz, 2H), 0.95 (t, J = 7.4 Hz, 3H). |
| 280 | C | 4.402 | 524.20 | 1H NMR (400 MHz, Methanol-d4) d 8.19 (s, 1H), 7.66 (dd, J = 8.1, 1.1 Hz, 2H), |
| | | | | 7.62-7.54 (m, 3H), 7.25-7.20 (m, 2H), 3.32 (1H under solvent peak), 1.54 (d, J = |
| | | | | 6.6 Hz, 6H). |
| 281 | C | 4.143 | 526.20 | 1H NMR (400 MHz, Methanol-d4) d 8.18 (s, 1H), 7.68-7.63 (m, 2H), 7.57 (dd, J = |
| | | | | 9.2, 7.0 Hz, 1H), 7.55-7.51 (m, 2H), 7.24-7.18 (m, 2H), 4.58 (s, 2H), 3.42 (s, 3H). |
| 282 | C | 4.412 | 524.20 | 1H NMR (400 MHz, Methanol-d4) d 8.19 (s, 1H), 7.69-7.60 (m, 4H), 7.58 (dd, J = |
| | | | | 9.2, 7.0 Hz, 1H), 7.26-7.18 (m, 2H), 4.35-4.27 (m, 2H), 2.01-1.86 (m, 2H), 0.91 |
| | | | | (t, J = 7.4 Hz, 3H). |
| 283 | C | 4.145 | 494.20 | 1H NMR (400 MHz, Methanol-d4) d 8.20 (d, J = 0.5 Hz, 1H), 7.61 (td, J = 8.3, 5.6 |
| | | | | Hz, 1H), 7.52 (dt, J = 8.3, 1.3 Hz, 1H), 7.47-7.35 (m, 3H), 7.25-7.17 (m, 2H), 2.85 |
| | | | | (q, J = 7.6 Hz, 2H), 1.30 (q, J = 7.3 Hz, 3H). |
| 284 | C | 4.064 | 478.20 | 1H NMR (400 MHz, Methanol-d4) d 8.23 (d, J = 0.9 Hz, 1H), 7.61 (tt, J = 8.4, 6.1 |
| | | | | Hz, 1H), 7.49-7.40 (m, 2H), 7.34-7.24 (m, 2H), 7.24-7.16 (m, 2H), 2.85 (q, J = 7.6 |
| | | | | Hz, 2H), 1.31 (t, J = 7.6 Hz, 3H). |
| 285 | C | 3.915 | 464.20 | ¹H NMR (400 MHz, Methanol-d₄) δ 8.25 (t, J = 0.9 Hz, 1H), 7.77-7.69 (m, 2H), 7.61 |
| | | | | (tt, J = 8.6, 6.1 Hz, 1H), 7.33-7.23 (m, 2H), 7.26-7.18 (m, 2H), 4.08 (s, 3H). |
| 286 | C | 3.927 | 425.20 | ¹H NMR (400 MHz, Methanol-d₄) δ 8.16 (d, J = 0.5 Hz, 1H), 7.60 (td, J = 8.4, 5.6 |
| | | | | Hz, 1H), 7.55-7.49 (m, 2H), 7.39 (ddd, J = 9.2, 8.4, 1.4 Hz, 1H), 7.34-7.29 (m, |
| | | | | 2H), 7.20-7.13 (m, 2H), 6.28 (dd, J = 1.8, 0.9 Hz, 1H), 2.70-2.61 (m, 2H), 1.22- |
| | | | | 1.16 (m, 3H). |
| 287 | C | 3.838 | 409.20 | ¹H NMR (400 MHz, Methanol-d₄) δ 8.19 (d, J = 1.0 Hz, 1H), 7.60 (tt, J = 8.6, 6.1 |
| | | | | Hz, 1H), 7.54 (d, J = 1.9 Hz, 1H), 7.36-7.30 (m, 2H), 7.30-7.23 (m, 2H), 7.20- |
| | | | | 7.13 (m, 2H), 6.28 (dd, J = 1.9, 0.9 Hz, 1H), 2.66 (q, J = 7.5 Hz, 2H), 1.21 (t, |
| | | | | J = 7.6 Hz, 3H). |
| 288 | C | 3.057 | 428.40 | ¹H NMR (400 MHz, Methanol-d₄) δ 7.94 (d, J = 1.0 Hz, 1H), 7.59 (tt, J = 8.5, 6.1 |
| | | | | Hz, 1H), 7.31-7.20 (m, 2H), 7.07-6.95 (m, 4H), 4.61-4.49 (m, 1H), 3.42-3.32 (m, |
| | | | | 2H), 3.28-3.13 (m, 2H), 2.86 (s, 3H), 2.20-2.01 (m, 4H). |
| 289 | C | 4.166 | 494.20 | ¹H NMR (400 MHz, Methanol-d₄) δ 8.22 (s, 1H), 7.67-7.63 (m, 2H), 7.60 (dd, J = |
| | | | | 8.4, 5.6 Hz, 1H), 7.53 (dt, J = 8.3, 1.3 Hz, 1H), 7.40 (ddd, J = 9.6, 8.4, 1.4 |
| | | | | Hz, 1H), 7.26-7.19 (m, 2H), 4.39 (q, J = 7.2 Hz, 2H), 1.53 (t, J = 7.3 Hz, 3H). |
| 290 | C | 2.75 | 427.2 | |
| 291 | C | 3.520 | 442.20 | 1H NMR (400 MHz, Methanol-d4) d 8.16 (s, 1H), 8.00 (s, 1H), 7.68-7.63 (m, 2H), |
| | | | | 7.57 (dd, J = 9.2, 7.0 Hz, 1H), 7.41-7.34 (m, 2H), 7.23-7.17 (m, 2H), 2.82 (q, |
| | | | | J = 7.6 Hz, 2H), 1.29 (t, J = 7.6 Hz, 3H). |
| 292 | C | 4.043 | 525.20 | 1H NMR (400 MHz, Methanol-d4) d 8.15 (d, J = 1.0 Hz, 1H), 7.87 (q, J = 1.4 Hz, |
| | | | | 1H), 7.71-7.67 (m, 2H), 7.66-7.62 (m, 2H), 7.60-7.54 (m, 1H), 7.20-7.14 (m, 2H), |
| | | | | 5.34 (s, 2H), 3.41 (s, 3H). |
| 293 | C | 4.096 | 509.20 | ¹H NMR (400 MHz, Methanol-d₄) δ 8.15 (s, 1H), 7.74 (q, J = 1.3 Hz, 1H), 7.65 (dd, |
| | | | | J = 8.1, 1.1 Hz, 2H), 7.57 (dd, J = 9.2, 7.0 Hz, 1H), 7.56-7.47 (m, 2H), 7.22- |
| | | | | 7.14 (m, 2H), 4.13 (q, J = 7.3 Hz, 2H), 1.41 (t, J = 7.3 Hz, H). |
| 294 | C | 3.95 | 495.2 | ¹H NMR (400 MHz, Methanol-d₄) δ 8.13 (s, 1H), 7.66-7.59 (m, 3H), 7.59-7.50 (m, |
| | | | | 3H), 7.20-7.09 (m, 2H), 3.77 (s, 3H). |
| 295 | C | 3.122 | 444.20 | ¹H NMR (400 MHz, Methanol-d₄) δ 7.90 (s, 1H), 7.58 (td, J = 8.3, 5.6 Hz, 1H), |
| | | | | 7.49 (dt, J = 8.3, 1.4 Hz, 1H), 7.37 (td, J = 8.9, 8.3, 1.4 Hz, 1H), 7.03-6.98 |
| | | | | (m, 2H), 6.98-6.91 (m, 2H), 4.46-4.39 (m, 1H), 3.07 (ddd, J = 12.2, 8.5, 3.7 Hz, |
| | | | | 2H), 2.77 (d, J = 37.0 Hz, 2H), 2.60 (s, 3H), 2.07 (ddt, J = 12.4, 7.9, 3.5 Hz, 2H). |
| 296 | C | 3.317 | 407.20 | ¹H NMR (400 MHz, Methanol-d₄) δ 8.97 (d, J = 5.1 Hz, 1H), 8.22 (d, J = 0.9 Hz, |
| | | | | 1H), 7.65 (dd, J = 5.2, 0.9 Hz, 1H), 7.60 (ddt, J = 8.1, 6.3, 2.3 Hz, 1H), 7.58- |
| | | | | 7.53 (m, 2H), 7.31-7.24 (m, 2H), 7.24-7.17 (m, 2H), 2.46 (d, J = 0.9 Hz, 3H). |
| 297 | C | 4.168 | 539.20 | 1H NMR (400 MHz, Methanol-d4) d 8.18 (s, 1H), 7.70-7.62 (m, 2H), 7.61-7.50 (m, |
| | | | | 3H), 7.24-7.14 (m, 2H), 3.66 (s, 2H), 2.30 (s, 6H). |
| 298 | C | 4.056 | 510.20 | ¹H NMR (400 MHz, Methanol-d₄) δ 8.22-8.18 (m, 1H), 7.61 (td, J = 8.4, 5.6 Hz, |
| | | | | 1H), 7.56-7.49 (m, 3H), 7.40 (ddd, J = 9.3, 8.4, 1.4 Hz, 1H), 7.24-7.18 (m, 2H), |
| | | | | 4.58 (s, 2H), 3.42 (s, 3H). |
| 299 | C | 3.973 | 494.20 | ¹H NMR (400 MHz, Methanol-d₄) δ 8.23 (d, J = 0.9 Hz, 1H), 7.61 (tt, J = 8.6, 6.1 |
| | | | | Hz, 1H), 7.56-7.51 (m, 2H), 7.31-7.24 (m, 2H), 7.24-7.18 (m, 2H), 4.61, 4.58 (s, |
| | | | | 2H), 3.42 (s, 3H). |
| 300 | C | 3.426 | 458.20 | |
| 301 | C | 3.84 | 537.2 | ¹H NMR (400 MHz, Methanol-d₄) δ 8.22 (s, 1H), 8.14 (s, 1H), 7.67-7.60 (m, 2H), |
| | | | | 7.56 (dd, J = 9.1, 7.0 Hz, 1H), 7.43-7.34 (m, 2H), 7.20-7.11 (m, 2H), 5.57 (ddd, |
| | | | | J = 7.5, 6.1, 1.4 Hz, 1H), 5.01 (t, J = 7.3 Hz, 2H), 4.85 (t, J = 7.3 Hz, 2H). |
| 302 | C | 4.084 | 478.20 | 1H NMR (400 MHz, Methanol-d4) d 8.25 (d, J = 1.0 Hz, 1H), 7.68-7.63 (m, 2H), 7.60 |
| | | | | (dt, J = 8.6, 6.1 Hz, 1H), 7.32-7.25 (m, 2H), 7.25-7.20 (m, 2H), 4.39 (q, J = 7.2 |
| | | | | Hz, 2H), 1.53 (t, J = 7.2 Hz, 3H). |
| 303 | C | 4.008 | 493.20 | ¹H NMR (400 MHz, Methanol-d₄) δ 8.18 (d, J = 0.5 Hz, 1H), 7.74 (q, J = 1.2 Hz, |
| | | | | 1H), 7.61 (td, J = 8.4, 5.6 Hz, 1H), 7.55-7.48 (m, 3H), 7.40 (ddd, J = 9.3, 8.4, |
| | | | | 1.4 Hz, 1H), 7.22-7.15 (m, 2H), 4.14 (q, J = 7.3 Hz, 2H), 1.41 (t, J = 7.3 Hz, 3H). |
| 304 | C | 3.928 | 477.20 | ¹H NMR (400 MHz, Methanol-d₄) δ 8.21 (d, J = 0.9 Hz, 1H), 7.74 (q, J = 1.2 Hz, |
| | | | | 1H), 7.61 (tt, J = 8.6, 6.1 Hz, 1H), 7.55-7.49 (m, 2H), 7.31-7.24 (m, 2H), 7.22- |
| | | | | 7.16 (m, 2H), 4.14 (q, J = 7.3 Hz, 2H), 1.41 (t, J = 7.3 Hz, 3H). |
| 305 | C | 3.867 | 479.20 | ¹H NMR (400 MHz, Methanol-d₄) δ 8.18 (d, J = 0.5 Hz, 1H), 7.65 (q, J = 1.3 Hz, |
| | | | | 1H), 7.64-7.55 (m, 3H), 7.52 (dt, J = 8.3, 1.3 Hz, 1H), 7.40 (ddd, J = 9.2, 8.4, |
| | | | | 1.4 Hz, 1H), 7.21-7.15 (m, 2H), 3.79 (s, 3H). |
| 306 | C | 3.652 | 552.20 | 1H NMR (400 MHz, Methanol-d4) d 8.16 (s, 1H), 7.79 (q, J = 1.3 Hz, 1H), 7.65 (dd, |
| | | | | J = 8.1, 1.0 Hz, 2H), 7.57 (dd, J = 9.2, 7.0 Hz, 1H), 7.55-7.51 (m, 2H), 7.21- |
| | | | | 7.16 (m, 2H), 4.21 (t, J = 6.8 Hz, 2H), 2.66 (t, J = 6.8 Hz, 2H), 2.19 (s, 6H). |
| 307 | C | 4.01 | 539.2 | 1H NMR (400 MHz, Methanol-d₄) δ 8.13 (s, 1H), 7.72 (q, J = 1.3 Hz, 1H), 7.66-7.59 |
| | | | | (m, 2H), 7.59-7.47 (m, 3H), 7.21-7.10 (m, 2H), 4.22 (t, J = 5.2 Hz, 2H), 3.67 (t, |
| | | | | J = 5.2 Hz, 2H), 3.30(s, , 3H). |
| 308 | C | 3.786 | 463.20 | ¹H NMR (400 MHz, Methanol-d₄) δ 8.21 (t, J = 0.9 Hz, 1H), 7.65 (p, J = 1.1 Hz, |
| | | | | 1H), 7.59-7.56 (m, 2H), 7.31-7.23 (m, 2H), 7.21-7.14 (m, 2H), 3.79 (s, 3H). |
| 309 | C | 3.495 | 424.20 | ¹H NMR (400 MHz, Methanol-d₄) δ 8.23 (s, 1H), 7.99 (s, 1H), 7.66-7.56 (m, 1H), |
| | | | | 7.53 (d, J = 8.2 Hz, 2H), 7.27 (td, J = 8.7, 7.4, 1.7 Hz, 2H), 7.21 (d, J = 8.4 |
| | | | | Hz, 2H), 4.80 (s, 1H), 1.51 (d, J = 6.6 Hz, 6H). |
| 310 | C | 3.740 | 466.20 | 1H NMR (400 MHz, Methanol-d4) d 8.11 (d, J = 0.9 Hz, 1H), 7.99 (d, J = 0.9 Hz, |
| | | | | 1H), 7.63-7.58 (m, 2H), 7.56-7.50 (m, 1H), 7.50-7.44 (m, 2H), 7.16-7.11 (m, 2H), |
| | | | | 4.12 (q, J = 7.3 Hz, 2H), 1.42-1.30 (m, 3H). |
| 311 | C | 4.23 | 523.2 | ¹H NMR (400 MHz, Methanol-d₄) δ 7.69 (s, 1H), 7.62 (dd, J = 8.1, 1.1 Hz, 2H), |
| | | | | 7.57-7.48 (m, 2H), 7.38 (s, 1H), 6.97 (d, J = 1.7 Hz, 1H), 2.65 (s, 3H). |
| 312 | C | 4.39 | 537.2 | ¹H NMR (400 MHz, Methanol-d₄) δ 8.14 (s, 1H), 7.74-7.52 (m, 3H), 7.52-7.41 (m, |
| | | | | 2H), 7.21-7.09 (m, 2H), 4.09 (t, J = 7.4 Hz, 2H), 1.71 (dd, J = 8.5, 7.4 Hz, 2H), |
| | | | | 1.38-1.14 (m, 2H), 0.86 (t, J = 7.4 Hz, 3H). |
| 313 | A | 2.785 | 487.2 | 1H NMR (400 MHz, cd3od) δ 8.09 (s, 1H), 7.60 (d, J = 7.2 Hz, 2H), 7.51 (dd, J = |
| | | | | 18.2, 7.8 Hz, 3H), 7.04 (d, J = 8.4 Hz, 2H), 3.90-3.51 (m, 4H), 3.43 (t, J = 10.0 |
| | | | | Hz, 1H), 2.92 (d, J = 42.3 Hz, 1H), 2.30 (d, J = 39.3 Hz, 6H), 1.83 (s, 1H). |
| 314 | C | 2.894 | 487.2 | 1H NMR (400 MHz, cd3od) δ 8.09 (s, 1H), 7.64-7.58 (m, 2H), 7.51 (dd, J = 17.9, |
| | | | | 7.8 Hz, 3H), 7.06 (s, 2H), 3.90-3.51 (m, 4H), 3.45 (t, J = 10.0 Hz, 1H), 2.96 (d, |
| | | | | J = 45.5 Hz, 1H), 2.33 (d, J = 22.3 Hz, 6H), 1.83 (s, 1H). |
| 315 | B | 3.419 | 462.2 | 1H NMR (400 MHz, cd3od) δ 8.09 (s, 1H), 7.61 (d, J = 8.6 Hz, 2H), 7.56-7.46 (m, |
| | | | | 3H), 7.05 (d, J = 8.7 Hz, 2H), 5.44-5.07 (m, 1H), 3.73 (d, J = 14.0 Hz, 4H), 2.20 |
| | | | | (s, 2H). |
| 316 | B | 3.420 | 462.2 | 1H NMR (400 MHz, cd3od) δ 8.09 (s, 1H), 7.64-7.57 (m, 2H), 7.53 (dd, J = 9.2, 6.9 |
| | | | | Hz, 3H), 7.05 (d, J = 8.7 Hz, 2H), 5.43-5.08 (m, 1H), 3.75 (t, J = 20.5 Hz, 4H), |
| | | | | 2.08 (d, J = 47.0 Hz, 2H). |
| 317 | C | 2.741 | 459.2 | 1H NMR (400 MHz, cd3od) δ 8.08 (s, 1H), 7.63-7.61 (m, 1H), 7.60 (s, 1H), 7.52 (d, |
| | | | | J = 8.9 Hz, 3H), 7.06 (d, J = 8.6 Hz, 2H), 3.93 (s, 3H), 3.83-3.77 (m, 1H), 3.76- |
| | | | | 3.60 (m, 3H), 2.37 (d, J = 10.1 Hz, 1H), 2.05 (s, 1H). |
| 318 | C | 2.808 | 459.2 | 1H NMR (400 MHz, cd3od) δ 8.08 (s, 1H), 7.61 (d, J = 8.7 Hz, 2H), 7.52 (dd, J = |
| | | | | 9.2, 3.0 Hz, 3H), 7.05 (d, J = 8.6 Hz, 2H), 3.90-3.59 (m, 5H), 3.51 (s, 2H), 2.33 |
| | | | | (s, 1H), 1.98-1.93 (m, 1H). |
| 319 | C | 2.787 | 459.2 | 1H NMR (400 MHz, cd3od) δ 8.08 (s, 1H), 7.60 (d, J = 7.4 Hz, 2H), 7.54-7.48 (m, |
| | | | | 3H), 7.05 (d, J = 8.6 Hz, 2H), 3.88-3.73 (m, 3H), 3.69-3.61 (m, 2H), 3.49 (s, 2H), |
| | | | | 2.25 (s, 1H), 1.95 (t, J = 6.7 Hz, 1H). |
| 320 | C | 3.68 | 434.2 | 1H NMR (400 MHz, Methanol-d₄) δ 7.69 (s, 1H), 7.62 (dd, J = 8.1, 1.1 Hz, 2H), |
| | | | | 7.57-7.48 (m, 2H), 7.38 (s, 1H), 6.97 (d, J = 1.7 Hz, 1H), 2.65 (s, 3H). |
| 321 | C | 3.4 | 425.2 | 1H NMR (400 MHz, Methanol-d₄) δ 9.56 (s, 1H), 9.12 (d, J = 5.7 Hz, 1H), 8.17 (s, |
| | | | | 1H), 8.05 (dd, J = 5.8, 2.5 Hz, 1H), 7.96-7.82 (m, 2H), 7.64 (dd, J = 8.1, 1.1 Hz, |
| | | | | 2H), 7.56 (dd, J = 9.2, 7.0 Hz, 1H), 7.30-7.10 (m, 2H). |
| 322 | C | 3.67 | 453.2 | 1H NMR (400 MHz, Methanol-d₄) δ 9.09 (s, 1H), 8.97 (s, 1H), 8.13 (s, 1H), 7.69- |
| | | | | 7.45 (m, 3H), 7.45-7.29 (m, 2H), 7.25-7.00 (m, 2H), 2.84 (q, J = 5.2 Hz, 2H), |
| | | | | 1.22 (t, J = 5.2 Hz, 3H) |
| 323 | C | 3.717 | 559.2 | |
| 324 | C | 3.718 | 559.2 | |
| 325 | C | 3.717 | 559.2 | |
| 326 | C | 3.363 | 428.20 | 1H NMR (400 MHz, Methanol-d4) d 8.10 (s, 1H), 7.94 (s, 1H), 7.63-7.57 (m, 2H), |
| | | | | 7.53 (dd, J = 9.2, 7.0 Hz, 1H), 7.37 (d, J = 8.6 Hz, 2H), 7.20-7.13 (m, 2H), 2.44 |
| | | | | (s, 3H). |
| 327 | C | 3.401 | 472.20 | 1H NMR (400 MHz, Methanol-d4) d 8.11 (s, 1H), 7.99 (s, 1H), 7.61 (d, J = 7.5 Hz, |
| | | | | 2H), 7.53 (dd, J = 9.2, 7.0 Hz, 1H), 7.40-7.35 (m, 2H), 7.18-7.13 (m, 2H), 3.70 (t, |
| | | | | J = 6.3 Hz, 2H), 3.24 (s, 3H), 3.01 (t, J = 6.3 Hz, 2H). |
| 328 | C | 4.066 | 527.20 | 1H NMR (400 MHz, Methanol-d4) d 8.28 (d, J = 2.9 Hz, 1H), 8.19 (s, 1H), 7.74 (d, |
| | | | | J = 8.9 Hz, 1H), 7.67-7.59 (m, 3H), 7.54 (dd, J = 9.2, 7.0 Hz, 1H), 4.99 (s, 2H), |
| | | | | 3.42 (s, 3H). |
| 329 | C | 4.253 | 511.20 | 1H NMR (400 MHz, Methanol-d4) d 8.30-8.27 (m, 1H), 8.19 (s, 1H), 7.70-7.63 (m, |
| | | | | 2H), 7.63-7.59 (m, 2H), 7.54 (dd, J = 9.2, 7.0 Hz, 1H), 3.15 (q, J = 7.5 Hz, 2H), |
| | | | | 1.32 (t, J = 7.5 Hz, 3H). |
| 330 | C | 4.116 | 540.20 | 1H NMR (400 MHz, Methanol-d4) d 8.12 (s, 1H), 7.63-7.58 (m, 2H), 7.53 (dd, J = |
| | | | | 9.2, 7.0 Hz, 1H), 7.46-7.36 (m, 2H), 7.20-7.12 (m, 2H), 3.72 (t, J = 6.2 Hz, 2H), |
| | | | | 3.25 (s, 3H), 3.04 (t, J = 6.2 Hz, 2H). |
| 331 | A | 2.806 | 513.2 | |
| 332 | A | 2.803 | 513.2 | |
| 333 | B | 3.22 | 464.2 | 1H NMR (400 MHz, Methanol-d4) δ 8.50 (dd, J = 7.1, 1.2 Hz, 1H), 8.18 (s, 1H), |
| | | | | 7.81-7.73 (m, 3H), 7.68-7.60 (m, 2H), 7.56 (dd, J = 9.2, 7.0 Hz, 1H), 7.48 (ddd, |
| | | | | J = 9.3, 6.6, 1.1 Hz, 1H), 7.31-7.23 (m, 2H), 7.05 (td, J = 6.8, 1.1 Hz, 1H). |
| 334 | E | 0.78 | 482 | — |
| 335 | C | 4.065 | 528.20 | 1H NMR (400 MHz, Methanol-d4) d 8.67 (s, 1H), 7.92 (d, J = 9.5 Hz, 1H), 7.65-7.59 |
| | | | | (m, 2H), 7.54 (dd, J = 9.3, 7.1 Hz, 2H), 5.03 (s, 2H), 3.42 (s, 3H). |
| 336 | C | 4.297 | 512.20 | 1H NMR (400 MHz, Methanol-d4) d 8.66 (d, J = 0.9 Hz, 1H), 7.87 (d, J = 9.5 Hz, |
| | | | | 1H), 7.64-7.59 (m, 2H), 7.57-7.51 (m, 2H), 3.21 (q, J = 7.5 Hz, 2H), 1.36 (t, |
| | | | | J = 7.5 Hz, 3H). |
| 337 | C | 3.355 | 415.20 | 1H NMR (400 MHz, Methanol-d4) d 9.16 (s, 1H), 8.25 (dd, J = 2.9, 0.7 Hz, 1H), |
| | | | | 8.15 (s, 1H), 8.11 (s, 1H), 7.80 (dd, J = 8.9, 0.7 Hz, 1H), 7.65 (d, J = 2.9 Hz, |
| | | | | 1H), 7.64-7.59 (m, 2H), 7.54 (dd, J = 9.2, 7.0 Hz, 1H). |
| 338 | C | 3.639 | 525.20 | 1H NMR (400 MHz, Methanol-d4) d 8.09 (s, 1H), 7.72 (q, J = 1.3 Hz, 1H), 7.64-7.58 |
| | | | | (m, 2H), 7.57-7.48 (m, 3H), 7.18-7.08 (m, 2H), 4.13 (t, J = 5.3 Hz, 2H), 3.79 (dd, |
| | | | | J = 5.8, 4.9 Hz, 2H). |
| 339 | C | 4.198 | 510.20 | 1H NMR (400 MHz, Methanol-d4) d 8.45 (dd, J = 3.0, 0.7 Hz, 1H), 8.23 (s, 1H), |
| | | | | 7.95-7.91 (m, 1H), 7.72 (q, J = 1.2 Hz, 1H), 7.66 (dd, J = 8.1, 1.1 Hz, 2H), 7.62- |
| | | | | 7.54 (m, 2H), 4.59 (q, J = 7.2 Hz, 2H), 1.44 (t, J = 7.1 Hz, 3H). |
| 340 | C | 3.5 | 439.2 | |
| 341 | C | 3.65 | 456.2 | 1H NMR (400 MHz, Methanol-d₄) δ 8.14 (s, 1H), 7.98 (d, J = 0.6 Hz, 1H), 7.66-7.60 |
| | | | | (m, 2H), 7.56 (dd, J = 9.2, 6.9 Hz, 1H), 7.36-7.30 (m, 2H), 7.26-7.11 (m, 2H), |
| | | | | 3.13 (sept, J = 6.8 Hz, 1H), 1.27 (d, J = 6.8 Hz, 6H). |
| 342 | C | 3.1 | 483.2 | 1H NMR (400 MHz, Methanol-d₄) δ 8.19-8.15 (m, 1H), 8.13 (s, 1H), 7.65 (d, J = 1.5 |
| | | | | Hz, 1H), 7.63 (d, J = 0.7 Hz, 1H), 7.56 (dd, J = 9.2, 7.0 Hz, 1H), 7.46-7.40 (m, |
| | | | | 2H), 7.20-7.14 (m, 2H), 5.32-5.24 (m, 1H), 4.67-4.60 (m, 1H), 4.49-4.40 (m, 2H), |
| | | | | 4.23-4.16 (m, 1H), 1.90 (s, 3H). |
| 343 | C | 3.47 | 443.2 | 1H NMR (400 MHz, Methanol-d₄) δ 8.30 (dd, J = 2.3, 1.3 Hz, 1H), 8.20 (s, 1H), |
| | | | | 7.96 (s, 1H), 7.68-7.64 (m, 2H), 7.63 (d, J = 0.7 Hz, 1H), 7.56 (dd, J = 9.2, 6.9 |
| | | | | Hz, 2H), 3.13 (q, J = 7.5 Hz, 2H), 1.31 (t, J = 7.5 Hz, 3H). |
| 344 | D | 2.96 | 454.2 | ¹H NMR (400 MHz, Methanol-d₄) δ 8.35 (dd, J = 2.9, 0.7 Hz, 1H), 8.14 (s, 1H), |
| | | | | 7.71 (dd, J = 8.7, 0.7 Hz, 1H), 7.66-7.61 (m, 2H), 7.58-7.53 (m, 3H), 7.51 (dd, |
| | | | | J = 8.7, 2.9 Hz, 1H), 7.35 (ddd, J = 8.3, 7.4, 1.8 Hz, 1H), 7.09 (dd, J = 8.4, |
| | | | | 1.0 Hz, 1H), 7.03 (td, J = 7.5, 1.1 Hz, 1H), 3.84 (s, 3H). |
| 345 | D | 3.32 | 431.2 | ¹H NMR (400 MHz, Methanol-d₄) δ 8.15 (s, 1H), 8.13 (dd, J = 5.6, 0.7 Hz, 1H), |
| | | | | 8.05 (s, 1H), 7.63 (d, J = 1.5 Hz, 1H), 7.61 (d, J = 0.7 Hz, 1H), 7.59-7.48 (m, |
| | | | | 2H), 4.10-4.04 (m, 2H), 2.63 (dd, J = 8.5, 7.6 Hz, 2H), 2.19-2.09 (m, 2H). |
| 346 | D | 3.57 | 428.2 | ¹H NMR (400 MHz, Methanol-d₄) δ 8.23 (s, 1H), 8.09 (dd, J = 2.9, 0.6 Hz, 1H), |
| | | | | 7.97 (dd, J = 2.4, 0.7 Hz, 1H), 7.72 (dd, J = 1.9, 0.7 Hz, 1H), 7.65 (d, J = 1.5 |
| | | | | Hz, 1H), 7.63 (d, J = 0.7 Hz, 1H), 7.56 (dd, J = 9.2, 7.0Hz, 1H), 7.48 (dd, J = 2.8, |
| | | | | 0.8 Hz, 1H), 6.51 (dd, J = 2.5, 1.9 Hz, 1H), 2.25 (t, J = 0.6 Hz, 3H). |
| 347 | C | 3.296 | 470.20 | ¹H NMR (400 MHz, Methanol-d₄) δ 8.13-8.08 (m, 1H), 8.05 (q, J = 0.7 Hz, 1H), |
| | | | | 7.63-7.58 (m, 2H), 7.57-7.48 (m, 1H), 7.24 (dd, J = 8.9, 1.8 Hz, 2H), 7.18-7.10 |
| | | | | (m, 2H), 4.88 (dqd, J = 6.8, 5.8, 1.2 Hz, 4H), 4.60-4.41 (m, 1H). |
| 348 | C | 3.136 | 444.20 | |
| 349 | C | 3.523 | 494.20 | ¹H NMR (400 MHz, Methanol-d₄) δ 8.47 (s, 1H), 8.11 (s, 1H), 7.61 (dd, J = 8.1, |
| | | | | 1.1 Hz, 2H), 7.53 (dd, J = 9.2, 7.0 Hz, 1H), 7.41-7.33 (m, 2H), 7.18-7.04 (m, |
| | | | | 2H), 5.56 (ddd, J = 13.4, 7.4, 6.1 Hz, 1H), 5.02-4.94 (m, 2H), 4.80 (under MeOD |
| | | | | signal, m, 2H). |
| 350 | C | 3.23 | 440.2 | 1H NMR (400 MHz, Methanol-d₄) δ 9.21 (s, 1H), 9.09 (s, 1H), 8.57-8.48 (m, 1H), |
| | | | | 8.22 (s, 1H), 7.74-7.48 (m, 5H), 2.54 (s, 3H). |
| 351 | C | 3.39 | 454.2 | ¹H NMR (400 MHz, Methanol-d₄) δ 9.18 (s, 1H), 8.52 (s, 1H), 8.22 (s, 1H), 7.73- |
| | | | | 7.49 (m, 6H), 2.96 (q, J = 7.6 Hz, 2H), 1.22 (t, J = 7.6 Hz, 3H). |
| 352 | C | 3.2 | 426.2 | 1H NMR (400 MHz, Methanol-d₄) δ 9.81 (s, 1H), 9.19 (s, 2H), 8.57-8.49 (m, 1H), |
| | | | | 8.24 (s, 1H), 8.11-8.02 (m, 1H), 7.65 (dd, J = 8.1, 1.1 Hz, 2H), 7.61-7.48 (m, 2H). |
| 353 | C | 2.81 | 364.2 | |
| 354 | A | 2.723 | 490.2 | |
| 355 | C | 3.335 | 463.2 | 1H NMR (400 MHz, MeOD) δ 8.37 (d, J = 2.6 Hz, 1H), 8.24 (s, 1H), 7.83 (dd, J = |
| | | | | 12.5, 8.7 Hz, 1H), 7.67 (d, J = 1.5 Hz, 1H), 7.65 (d, J = 0.7 Hz, 1H), 7.61-7.49 |
| | | | | (m, 2H), 5.40 (d, J = 10.3 Hz, 1H), 5.26 (d, J = 10.3 Hz, 1H), 4.22-3.65 (m, 5H), |
| | | | | 2.35-2.03 (m, 2H). |
| 356 | A | 2.776 | 515.2 | |
| 357 | A | 2.714 | 460.2 | |
| 358 | A | 3.507 | 447.2 | 1H NMR (400 MHz, MeOD) δ 8.35 (d, J = 2.8 Hz, 1H), 8.23 (s, 1H), 7.75 (d, J = 8.7 |
| | | | | Hz, 1H), 7.68-7.62 (m, 2H), 7.61-7.48 (m, 2H), 3.84-3.77 (m, 2H), 3.67-3.60 (m, |
| | | | | 2H), 2.01-1.92 (m, 4H). |
| 359 | C | 3.008 | 335.20 | 1H NMR (400 MHz, Methanol-d4) d 7.59 (s, 1H), 7.49 (d, J = 0.9 Hz, 1H), 7.45 (t, |
| | | | | J = 1.3 Hz, 1H), 7.43 (dd, J = 2.7, 1.4 Hz, 1H), 7.35 (d, J = 0.9 Hz, 1H), 7.35- |
| | | | | 7.28 (m, 1H), 3.80 (s, 3H). |
| 360 | C | 3.057 | 429.20 | ¹H NMR (400 MHz, Methanol-d₄) δ 8.51 (s, 1H), 8.25 (s, 1H), 8.02 (d, J = 8.7 Hz, |
| | | | | 1H), 7.70-7.64 (m, 2H), 7.64-7.53 (m, 3H), 4.08 (s, 3H). |
| 361 | C | 3.334 | 482.20 | 1H NMR (400 MHz, Methanol-d4) d 8.15 (s, 1H), 8.05 (s, 1H), 7.70-7.61 (m, 2H), |
| | | | | 7.64-7.51 (m, 3H), 7.21-7.13 (m, 2H), 4.20 (dd, J = 5.7, 4.8 Hz, 2H), 3.82 (dd, |
| | | | | J = 5.8, 4.8 Hz, 2H). |
| 362 | A | 4.108 & | 518.2 | |
| | | 4.167 | | |
| 363 | A | 2.686 | 475.4 | |
| 364 | A | 2.742 | 502.2 | 1H NMR (400 MHz, MeOD) δ 8.34 (dd, J = 2.8, 0.8 Hz, 1H), 8.22 (s, 1H), 7.67 (d, |
| | | | | J = 1.6 Hz, 1H), 7.65 (d, J = 0.7 Hz, 1H), 7.61-7.52 (m, 3H), 3.69 (d, J = 40.4 |
| | | | | Hz, 4H), 2.70 (d, J = 28.8 Hz, 4H), 1.74 (tt, J = 6.6, 3.7 Hz, 1H), 0.58-0.40 (m, 4H). |
| 365 | A | 3.263 | 461.2 | |
| 366 | A | 2.759 | 490.3 | |
| 367 | A | 3.632 | 465.2 | |
| 368 | A | 2.753 | 460.2 | |
| 369 | A | 4.107 & | 517.2 | |
| | | 4.162 | | |
| 370 | A | 2.779 | 476.2 | |
| 371 | C | 3.796 | 512.20 | 1H NMR (400 MHz, Methanol-d4) d 8.17 (s, 1H), 7.69-7.62 (m, 2H), 7.61-7.52 (m, |
| | | | | 3H), 7.25-7.17 (m, 2H), 4.70 (s, 2H). |
| 372 | C | 3.201 | 361.20 | 1H NMR (400 MHz, Methanol-d4) d 7.55 (d, J = 1.0 Hz, 1H), 7.51 (s, 1H), 7.51-7.41 |
| | | | | (m, 2H), 7.17 (ddd, J = 8.5, 6.1, 1.2 Hz, 2H), 4.11 (q, J = 7.0 Hz, 2H), 3.85 (s, |
| | | | | 3H), 1.29 (t, J = 7.0 Hz, 3H). |
| 373 | C | 3.700 | 526.20 | 1H NMR (400 MHz, Methanol-d4) d 8.42 (dd, J = 3.0, 0.6 Hz, 1H), 8.23 (s, 1H), |
| | | | | 7.95 (dd, J = 8.8, 0.6 Hz, 1H), 7.72 (q, J = 1.2 Hz, 1H), 7.66 (dd, J = 8.1, 1.1 |
| | | | | Hz, 2H), 7.58 (ddd, J = 9.2, 5.2, 2.2 Hz, 2H), 4.65 (t, J = 5.3 Hz, 2H), 3.91 (t, J = |
| | | | | 5.3 Hz, 2H). |
| 374 | C | 3.718 | 443.20 | 1H NMR (400 MHz, Methanol-d4) d 8.31 (s, 1H), 8.22 (d, J = 1.0 Hz, 1H), 7.84 (s, |
| | | | | 1H), 7.79 (d, J = 1.0 Hz, 1H), 7.67-7.61 (m, 2H), 7.56 (dd, J = 9.2, 7.0 Hz, 1H), |
| | | | | 2.78 (s, 3H), 2.54 (t, J = 0.7 Hz, 3H). |
| 375 | C | 3.478 | 430.2 | |
| 376 | B | 3.276 | 478 | — |
| 377 | C | 3.11 | 351.2 | 1H NMR (400 MHz, Methanol-d₄) δ 7.64-7.56 (m, 3H), 7.55-7.49 (m, 2H), 7.37 (d, |
| | | | | J = 0.9 Hz, 1H), 3.82 (S, 3H). |
| 378 | C | 3.48 | 379.2 | 1H NMR (400 MHz, Methanol-d₄) δ 7.64-7.46 (m, 5H), 7.39 (d, J = 1.0 Hz, 1H), 4.44 |
| | | | | (sept, J = 6.7 Hz, 1H), 1.46 (d, J = 6.7 Hz, 6H). |
| 379 | C | 3.45 | 352.2 | 1H NMR (400 MHz, Methanol-d₄) δ 8.10 (s, 1H), 7.67-7.47 (m, 3H), 7.27 (s, 1H), |
| | | | | 4.03 (s, 3H). |
| 380 | C | 3.07 | 3352.2 | 1H NMR (400 MHz, Methanol-d₄) δ 7.95 (s, 1H), 7.68-7.43 (m, 5H), 4.05 (s, 3H). |
| 381 | C | 2.84 | 378.2 | 1H NMR (400 MHz, Methanol-d₄) δ 8.15 (d, J = 0.8 Hz, 1H), 7.64 (dd, J = 8.1, 1.2 |
| | | | | Hz, 2H), 7.56 (dd, J = 9.2, 6.9 Hz, 2H), 7.49 (d, J = 7.4 Hz, 1H), 6.22 (dd, |
| | | | | J = 7.4, 1.4 Hz, 1H), 3.49 (s, 3H). |
| 382 | C | 2.963 | 514.2 | |
| 383 | C | 3.664 | 500.2 | |
| 384 | C | 3.26 | 445.2 | 1H NMR (400 MHz, Methanol-d₄) δ 8.22 (d, J = 3.0 Hz, 1H), 8.11 (s, 1H), 7.64 (d, |
| | | | | J = 1.5 Hz, 1H), 7.62 (d, J = 0.7 Hz, 1H), 7.56-7.50 (m, 2H), 7.35 (dd, J = 8.8, |
| | | | | 0.7 Hz, 1H), 3.76 (t, J = 5.8 Hz, 2H), 2.53 (t, J = 5.8 Hz, 2H), 2.01-1.89 (m, 4H). |
| 385 | C | 3.847 | 513.20 | 1H NMR (400 MHz, Methanol-d4) d 8.30 (dd, J = 2.9, 0.7 Hz, 1H), 8.19 (s, 1H), |
| | | | | 7.80 (dd, J = 8.9, 0.7 Hz, 1H), 7.67 (dd, J = 8.9, 2.9 Hz, 1H), 7.64-7.59 (m, 2H), |
| | | | | 7.54 (dd, J = 9.2, 7.0 Hz, 1H), 5.00 (s, 2H). |
| 386 | C | 3.544 | 432.20 | no nmr |
| 387 | C | 3.362 | 483.20 | 1H NMR (400 MHz, Methanol-d4) d 8.37 (dd, J = 2.9, 0.7 Hz, 1H), 8.18 (s, 1H), |
| | | | | 7.93 (s, 1H), 7.91 (dd, J = 8.7, 0.7 Hz, 1H), 7.64-7.57 (m, 2H), 7.56-7.50 (m, 2H), |
| | | | | 4.64 (t, J = 5.3 Hz, 2H), 3.86 (t, J = 5.3 Hz, 2H). |
| 388 | B | 3.21 | 479.2 | ¹H NMR (600 MHz, Methanol-d₄) δ 8.50 (d, J = 2.8 Hz, 1H), 8.28 (s, 1H), 7.76 (d, |
| | | | | J = 8.5 Hz, 1H), 7.69 (d, J = 9.2 Hz, 1H), 7.64 (d, J = 7.7 Hz, 2H), 7.63-7.61 |
| | | | | (m, 1H), 7.57 (dd, J = 8.9, 7.4 Hz, 1H), 7.43 (dd, J = 9.2, 6.7 Hz, 1H), 6.83 (d, |
| | | | | J = 6.7 Hz, 1H), 2.35 (s, 3H). |
| 389 | C | 2.93 | 378.2 | ¹H NMR (400 MHz, Methanol-d₄) δ 8.25 (d, J = 2.8 Hz, 1H), 7.72 (dd, J = 9.6, 2.8 |
| | | | | Hz, 1H), 7.63-7.56 (m, 3H), 7.56-7.46 (m, 1H), 6.56 (d, J = 9.6 Hz, 1H), 3.60 (s, 3H). |
| 390 | C | 3.19 | 441.2 | |
| 391 | C | 3.51 | 428.2 | ¹H NMR (400 MHz, Methanol-d₄) δ 8.17 (s, 1H), 7.68-7.51 (m, 4H), 7.46-7.33 (m, |
| | | | | 2H), 7.27-7.13 (m, 2H), 2.34 (d, J = 0.8 Hz, 3H). |
| 392 | C | 3.71 | 457.2 | |
| 393 | C | 3.76 | 458.2 | ¹H NMR (400 MHz, Methanol-d₄) δ 8.37 (dd, J = 2.9, 0.8 Hz, 1H), 8.26 (s, 1H), |
| | | | | 7.78-7.61 (m, 4H), 7.57 (dd, J = 9.2, 7.0 Hz, 1H), 3.77 (sept, J = 6.9 Hz, 1H), |
| | | | | 1.38 (d, J = 6.9 Hz, 6H). |
| 394 | C | 3.496 | 439.20 | 1H NMR (400 MHz, Methanol-d4) d 8.50 (s, 1H), 8.46 (d, J = 2.9 Hz, 1H), 8.41 (d, |
| | | | | J = 5.2 Hz, 1H), 8.22 (d, J = 1.9 Hz, 1H), 7.69-7.64 (m, 2H), 7.64-7.54 (m, 2H), |
| | | | | 7.50 (dd, J = 8.5, 1.8 Hz, 1H), 7.40 (d, J = 5.2 Hz, 1H), 2.43 (s, 3H). |
| 395 | C | 3.646 | 439.20 | 1H NMR (400 MHz, Methanol-d4) d 8.46-8.41 (m, 2H), 8.24 (s, 1H), 7.80 (ddd, J = |
| | | | | 7.6, 1.7, 0.8 Hz, 1H), 7.69-7.61 (m, 4H), 7.58 (dd, J = 9.2, 7.0 Hz, 1H), 7.37 |
| | | | | (dd, J = 7.7, 4.8 Hz, 1H), 2.43 (s, 3H). |
| 396 | C | 4.002 | 448.20 | |
| 397 | C | 3.785 | 414.20 | 1H NMR (400 MHz, Methanol-d4) d 8.42-8.37 (m, 2H), 7.96-7.92 (m, 2H), 7.84 (s, |
| | | | | 1H), 7.76 (d, J = 1.0 Hz, 1H), 7.67-7.62 (m, 2H), 7.57 (dd, J = 9.2, 7.0 Hz, 1H), |
| | | | | 7.27 (td, J = 5.1, 3.3 Hz, 1H). |
| 398 | C | 3.908 | 442.20 | 1H NMR (400 MHz, Methanol-d4) d 8.26 (dd, J = 3.1, 0.6 Hz, 1H), 8.18 (s, 1H), |
| | | | | 7.68-7.61 (m, 3H), 7.60-7.54 (m, 1H), 7.52 (dd, J = 8.8, 0.7 Hz, 1H), 6.06 (s, |
| | | | | 1H), 2.44 (d, J = 0.8 Hz, 3H), 2.26 (s, 3H). |
| 399 | C | 3.500 | 414.20 | |
| 400 | C | 3.355 | 457.20 | 1H NMR (400 MHz, Methanol-d4) d 8.72 (s, 1H), 8.45 (d, J = 2.9 Hz, 1H), 8.21 (d, |
| | | | | J = 0.5 Hz, 1H), 7.92 (dd, J = 8.7, 0.7 Hz, 1H), 7.65-7.58 (m, 2H), 7.58-7.49 (m, |
| | | | | 2H), 5.52 (p, J = 6.8 Hz, 1H), 1.51 (d, J = 6.7 Hz, 6H). |
| 401 | C | 3.273 | 462.20 | 1H NMR (400 MHz, Methanol-d4) d 8.59 (d, J = 0.6 Hz, 1H), 8.52 (d, J = 1.4 Hz, |
| | | | | 1H), 8.27 (t, J = 1.1 Hz, 1H), 7.62-7.57 (m, 2H), 7.52 (dd, J = 9.4, 6.9 Hz, 1H), |
| | | | | 3.74 (s, 8H). |
| 402 | C | 3.300 | 475.20 | 1H NMR (400 MHz, Methanol-d4) d 8.60 (s, 1H), 8.51 (d, J = 1.4 Hz, 1H), 8.28 (d, |
| | | | | J = 1.4 Hz, 1H), 7.64-7.57 (m, 2H), 7.53 (dd, J = 9.2, 6.9 Hz, 1H), 3.80-3.73 (m, |
| | | | | 4H), 2.54 (bs, 4H), 2.34 (s, 3H). |
| 403 | C | 3.46 | 464.2 | |
| 404 | C | 3.04 | 336.2 | 1H NMR (400 MHz, Methanol-d₄) δ 8.24 (s, 1H), 7.64-7.54 (m, 2H), 7.49 (dt, J = |
| | | | | 8.3, 1.3 Hz, 1H), 7.37 (ddd, J = 9.5, 8.5, 1.4 Hz, 1H), 3.76 (s, 3H). |
| 405 | C | 3.075 | 461.20 | |
| 406 | C | 3.16 | 352.2 | |
| 407 | C | 2.9 | 337.2 | |
| 408 | C | 3.229 | 388.20 | 1H NMR (400 MHz, Methanol-d4) d 8.51 (dd, J = 4.4, 1.4 Hz, 1H), 8.06 (s, 1H), |
| | | | | 7.95 (d, J = 9.2 Hz, 1H), 7.65 (dd, J = 8.1, 1.1 Hz, 2H), 7.57 (dd, J = 9.2, 7.0 |
| | | | | Hz, 1H), 7.54 (bs, 1H), 7.14 (dd, J = 9.2, 4.4 Hz, 1H). |
| 409 | C | 3.215 | 388.20 | 1H NMR (400 MHz, Methanol-d4) d 8.76 (dd, J = 7.1, 1.7 Hz, 1H), 8.41 (dd, J = |
| | | | | 4.0, 1.6 Hz, 1H), 8.19 (s, 1H), 7.76 (s, 1H), 7.62 (dd, J = 8.1, 1.1 Hz, 2H), |
| | | | | 7.54 (dd, J = 9.2, 7.0 Hz, 1H), 6.93 (dd, J = 7.1, 3.9 Hz, 1H). |
| 410 | C | 2.821 | 461.40 | |
| 411 | C | 3.7 | 442.2 | 1H NMR (400 MHz, Methanol-d₄) δ 8.15 (s, 1H), 7.69-7.50 (m, 4H), 7.43-7.27 (m, |
| | | | | 2H), 7.27-7.07 (m, 2H), 2.71 (q, J = 7.6, 2H), 1.22 (m, J = 7.6, 3H). |
| 412 | A | 3.6 | 478.2 | 1H NMR (400 MHz, Methanol-d₄) δ 8.12 (s, 1H), 7.55 (d, J = 8.1 Hz, 2H), 7.43-7.37 |
| | | | | (m, 2H), 7.10-7.05(m, 2H), 3.67 (d, J = 15.0 Hz, 9H). |
| 413 | A | 2.910 | 519.2 | |
| 414 | A | 3.22 | 460.2 | 1H NMR (400 MHz, Methanol-d₄) δ 8.63 (s, 1H), 8.17 (s, 1H), 7.56 (dd, J = 8.5, |
| | | | | 6.9 Hz, 4H), 7.23-7.17 (m, 2H), 4.19 (q, J = 7.3 Hz, 2H), 1.41 (t, J = 7.3 Hz, 3H). |
| 415 | C | 3.342 | 458.20 | |
| 416 | A | 3.66 | 478.2 | 1H NMR (400 MHz, Methanol-d₄) δ 8.15 (s, 1H), 7.67 (dd, J = 9.2, 5.0 Hz, 1H), |
| | | | | 7.55 (dd, J = 9.2, 8.4 Hz, 1H), 7.43-7.37 (m, 2H), 7.11-7.05 (m, 2H), 3.67 (m, 8H). |
| 417 | A | 2.93 | 519.2 | 1H NMR (400 MHz, Methanol-d₄) δ 8.17 (s, 1H), 7.69 (dd, J = 9.1, 5.0 Hz, 1H), |
| | | | | 7.57 (dd, J = 9.2, 8.3 Hz, 1H), 7.40 (d, J = 8.2 Hz, 2H), 7.14-7.07 (m, 2H), 3.33 |
| | | | | (p, J = 1.6 Hz, 1H), 2.98 (s, 3H), 2.58 (m, 4H), 2.13-2.00 (m, 2H), 1.98 (s, 3H), |
| | | | | 1.91 (m, 2H). |
| 418 | A | 3.42 | 460.2 | |
| 419 | C | 3.37 | 448.2 | 1H NMR (400 MHz, Methanol-d₄) δ 8.13 (d, J = 0.5 Hz, 1H), 7.81-7.71 (m, 2H), |
| | | | | 7.66-7.59 (m, 2H), 7.55 (ddd, J = 9.2, 7.0, 0.5 Hz, 1H), 7.09-7.00 (m, 2H), 3.58- |
| | | | | 3.49 (m, 4H), 3.38 (s, 3H). |
| 420 | C | 3.47 | 462.2 | 1H NMR (600 MHz, Methanol-d₄) δ 8.13 (s, 1H), 7.80-7.70 (m, 2H), 7.67-7.59 (m, |
| | | | | 2H), 7.55 (dd, J = 8.9, 7.4 Hz, 1H), 7.11-6.95 (m, 2H), 3.48 (t, J = 6.5 Hz, 2H), |
| | | | | 3.43 (t, J = 6.5 Hz, 2H), 3.34 (s, 3H), 1.86 (q, J = 6.5 Hz, 2H). |
| 421 | C | 3.33 | 436.2 | 1H NMR (400 MHz, Methanol-d₄) δ 8.15 (s, 1H), 7.85-7.70 (m, 2H), 7.70-7.46 (m, |
| | | | | 3H), 7.12-6.97 (m, 2H), 4.60 (t, J = 5.1 Hz, 1H), 4.48 (t, J = 5.1 Hz, 1H), 3.68 |
| | | | | (t, J = 5.1 Hz, 1H), 3.62 (t, J = 5.1 Hz, 1H). |
| 422 | C | 3.18 | 489.2 | 1H NMR (400 MHz, Methanol-d₄) δ 8.14 (s, 1H), 7.82-7.72 (m, 2H), 7.67-7.49 (m, |
| | | | | 3H), 7.11-7.00 (m, 2H), 3.55 (t, J = 6.8 Hz, 2H), 2.96-2.71 (m, 6H), 1.17 (t, J = |
| | | | | 7.2 Hz, 6H). |
| 423 | C | 2.91 | 501.2 | 1H NMR (400 MHz, Methanol-d₄) δ 8.10 (s, 1H), 7.70-7.59 (m, 2H), 7.55 (dd, J = |
| | | | | 9.2, 7.0 Hz, 1H), 7.45-7.32 (m, 2H), 7.14-6.99 (m, 2H), 3.20-2.86 (m, 3H), 2.57 |
| | | | | (s, 6H), 2.05-2.01 (m, 4H), 1.61-1.51 (m, 2H). |
| 424 | C | 3.13 | 446.2 | 1H NMR (400 MHz, Methanol-d₄) δ 7.85 (s, 1H), 7.61 (dd, J = 8.1, 1.1 Hz, 2H), |
| | | | | 7.53 (dd, J = 9.2, 7.0 Hz, 1H), 7.05-6.88 (m, 4H), 4.48 (dt, J = 6.9, 3.5 Hz, |
| | | | | 1H), 3.31-3.20 (m, 2H), 3.08-2.93 (m, 2H), 2.10-1.99 (m, 2H), 1.96-1.79 (m, 2H). |
| 425 | C | 3.43 | 448.2 | 1H NMR (400 MHz, Methanol-d₄) δ 8.14 (s, 1H), 7.74-7.41 (m, 5H), 7.24-6.92 (m, |
| | | | | 2H), 5.31 (dt, J = 6.1, 2.9 Hz, 1H), 4.79-3.99 (m, 4H). |
| 426 | C | 3.04 | 446.2 | ¹H NMR (400 MHz, Methanol-d₄) δ 8.13 (s, 1H), 7.70-7.47 (m, 5H), 7.14-6.96 (m, |
| | | | | 2H), 4.71-4.51 (m, 2H), 4.38 (brs, 1H), 4.19 (brs, 1H), 3.94 (brs, 1H). |
| 427 | B | 3.415 | 478.2 | ¹H NMR (400 MHz, Methanol-d₄) δ 8.80-8.74 (m, 1H), 8.44-8.36 (m, 1H), 8.29 (s, |
| | | | | 1H), 7.98-7.86 (m, 2H), 7.66 (dd, J = 8.2, 1.1 Hz, 1H), 7.58 (dd, J = 9.2, 7.0 |
| | | | | Hz, 1H), 7.45 (d, J = 8.4 Hz, 1H), 7.39-7.32 (m, 1H), 7.02 (d, J = 7.0 Hz, 1H), 2.46 |
| | | | | (s, 3H). |
| 428 | C | 3.69 | 433.2 | 1H NMR (400 MHz, Chloroform-d) δ 8.87 (brs, 1H), 8.51 (s, 1H), 7.94 (d, J = 3.0 |
| | | | | Hz, 1H), 7.57-7.29 (m, 4H), 6.78 (d, J = 8.7 Hz, 2H), 5.41 (brs, 1H), 4.02-3.78 |
| | | | | (m, 4H), 3.20-2.97 (m, 4H). |
| 429 | B | 3.298 | 479.2 | 1H NMR (400 MHz, Methanol-d4) δ 8.65 (s, 1H), 8.45 (dd, J = 2.4, 0.8 Hz, 1H), |
| | | | | 7.82 (dd, J = 8.6, 2.4 Hz, 1H), 7.69-7.61 (m, 3H), 7.55 (dd, J = 9.2, 7.0 Hz, |
| | | | | 1H), 7.40 (dd, J = 9.3, 6.7 Hz, 1H), 7.05 (dd, J = 8.6, 0.8 Hz, 1H), 6.79 (dt, J = |
| | | | | 6.7, 1.1 Hz, 1H), 2.36 (s, 3H). |
| 430 | C | 3.385 | 429.20 | ¹H NMR (600 MHz, Methanol-d₄) δ 8.63 (d, J = 1.1 Hz, 1H), 8.36 (d, J = 2.6 Hz, |
| | | | | 1H), 8.00 (s, 1H), 7.72 (ddd, J = 9.0, 2.9, 1.9 Hz, 1H), 7.64-7.60 (m, 2H), 7.54 |
| | | | | (ddt, J = 9.1, 7.6, 1.4 Hz, 1H), 7.05 (dd, J = 9.0, 2.6 Hz, 1H), 2.48 (d, J = 1.5 |
| | | | | Hz, 3H). |
| 431 | C | 3.541 | 443.20 | 1H NMR (600 MHz, Methanol-d4) d 8.65 (s, 1H), 8.52 (d, J = 2.1 Hz, 1H), 8.02 (s, |
| | | | | 1H), 7.85 (dd, J = 8.7, 2.2 Hz, 1H), 7.64-7.59 (m, 2H), 7.55 (dd, J = 8.9, 7.5 |
| | | | | Hz, 1H), 7.05 (d, J = 8.6 Hz, 1H), 4.30 (q, J = 7.2 Hz, 2H), 1.48 (t, J = 7.2 Hz, 3H). |
| 432 | C | 3.545 | 429.20 | 1H NMR (600 MHz, Methanol-d4) d 8.64 (d, J = 1.2 Hz, 1H), 8.37 (t, J = 2.4 Hz, |
| | | | | 1H), 7.74 (dt, J = 8.9, 2.4 Hz, 1H), 7.65-7.60 (m, 3H), 7.55 (ddd, J = 9.0, 7.4, |
| | | | | 2.1 Hz, 1H), 7.08 (dd, J = 8.9, 2.2 Hz, 1H), 2.35 (s, 3H). |
| 433 | C | 3.97 | 496.2 | 1H NMR (600 MHz, Methanol-d₄) δ 8.64 (s, 1H), 8.51 (d, J = 2.4 Hz, 1H), 7.85 (dd, |
| | | | | J = 8.7, 2.4 Hz, 1H), 7.71-7.59 (m, 3H), 7.55 (dd, J = 8.9, 7.4 Hz, 1H), 7.01 |
| | | | | (d, J = 8.7 Hz, 1H), 3.79 (s, 3H). |
| 434 | C | 3.79 | 430.2 | 1H NMR (400 MHz, Methanol-d₄) δ 8.95 (d, J = 2.2 Hz, 1H), 8.63 (s, 1H), 8.21 (dd, |
| | | | | J = 8.7, 2.3 Hz, 1H), 7.71-7.47 (m, 3H), 7.01 (d, J = 8.7 Hz, 1H), 4.40 (s, 3H). |
| 435 | C | 3.51 | 430.2 | 1H NMR (600 MHz, Methanol-d₄) δ 8.65 (d, J = 1.0 Hz, 1H), 8.42 (dd, J = 2.7, 0.7 |
| | | | | Hz, 1H), 7.79 (dd, J = 8.9, 2.7 Hz, 1H), 7.68-7.59 (m, 2H), 7.59-7.48 (m, 1H), |
| | | | | 7.11 (dd, J = 8.9, 0.8 Hz, 1H), 2.58 (s, 3H). |
| 436 | B | 3.098 | 480.2 | 1H NMR (400 MHz, Methanol-d4) δ 9.25 (s, 1H), 8.54 (d, J = 2.8 Hz, 1H), 8.28 (s, |
| | | | | 1H), 7.82 (d, J = 8.6 Hz, 1H), 7.76 (s, 1H), 7.66-7.61 (m, 3H), 7.59-7.54 (m, |
| | | | | 2H), 2.41 (d, J = 1.0 Hz, 3H). |
| 437 | C | 4.070 | 483.20 | ¹H NMR (400 MHz, Chloroform-d) δ 9.34 (s, 1H), 8.58 (s, 1H), 8.33 (d, J = 2.6 Hz, |
| | | | | 1H), 8.10 (d, J = 0.9 Hz, 1H), 7.58 (dd, J = 8.9, 2.6 Hz, 1H), 7.48 (dd, J = 8.1, |
| | | | | 0.9 Hz, 2H), 7.39 (dd, J = 9.0, 7.2 Hz, 1H), 6.89 (dd, J = 8.9, 0.7 Hz, 1H), 6.80 |
| | | | | (s, 1H), 5.44 (s, 1H). |
| 438 | C | 4.01 | 482.2 | ¹H NMR (400 MHz, Methanol-d₄) δ 8.32 (q, J = 0.9 Hz, 1H), 8.18 (s, 1H), 7.63 (dd, |
| | | | | J = 8.1, 1.1 Hz, 2H), 7.55 (dd, J = 9.2, 7.0 Hz, 1H), 7.49-7.35 (m, 2H), 7.27- |
| | | | | 7.11 (m, 2H). |
| 439 | C | 2.992 | 447.2 | |
| 440 | A | 2.780 | 475.2 | 1H NMR (600 MHz, cdcl3) δ 8.32 (d, J = 2.8 Hz, 1H), 8.18 (s, 1H), 7.58 (s, 1H), |
| | | | | 7.51 (d, J = 0.9 Hz, 1H), 7.49 (s, 1H), 7.42 (dd, J = 8.8, 7.4 Hz, 1H), 7.31 (dd, |
| | | | | J = 8.5, 2.9 Hz, 1H), 7.26 (s, 1H), 6.75 (s, 1H), 5.42 (s, 1H), 3.85(s, 2H), 3.62 |
| | | | | (d, J = 4.1 Hz, 6H), 3.57-3.52 (m, 2H). |
| 441 | C | 3.131 | 463.2 | 1H NMR (600 MHz, cdcl3) δ 8.34 (d, J = 2.8 Hz, 1H), 8.16 (s, 1H), 7.58 (s, 1H), |
| | | | | 7.52-7.43 (m, 2H), 7.41 (dd, J = 8.8, 7.5 Hz, 1H), 7.32-7.24 (m, 3H), 6.75 (s, |
| | | | | 1H), 5.28 (dtt, J = 56.5, 6.3, 3.5 Hz, 1H), 4.51 (dddd, J = 19.4, 10.4, 6.1, 1.7 |
| | | | | Hz, 1H), 4.37-4.22 (m, 2H), 4.17-4.07 (m, 1H), 3.66-3.57 (m, 2H). |
| 442 | A | 2.796 | 502.2 | 1H NMR (400 MHz, CDCl3) δ 9.13 (s, 1H), 8.62 (s, 1H), 8.35 (d, J = 2.4 Hz, 1H), |
| | | | | 7.66-7.60 (m, 1H), 7.52 (dd, J = 8.0, 0.9 Hz, 2H), 7.43 (dd, J = 9.0, 7.2 Hz, |
| | | | | 1H), 7.28 (s, 1H), 6.87-6.77 (m, 2H), 5.56 (s, 1H), 2.99 (s, 2H), 2.73-2.50 (m, |
| | | | | 2H), 2.36 (s, 6H), 1.99 (s, 2H), 1.61-1.45 (m, 2H). |
| 443 | A | 3.620 | 465.2 | 1H NMR (400 MHz, CDCl3) δ 9.24 (s, 1H), 8.55 (d, J = 36.2 Hz, 2H), 7.80 (d, J = |
| | | | | 8.6 Hz, 1H), 7.52 (dd, J = 8.1, 0.9 Hz, 2H), 7.43 (dd, J = 9.0, 7.1 Hz, 1H), 6.81 |
| | | | | (dd, J = 8.7, 0.8 Hz, 2H), 5.52 (s, 1H), 5.34 (d, J = 53.0 Hz, 2H), 4.08-3.58 (m, 4H). |
| 444 | A | 3.519 | 456.2 | 1H NMR (400 MHz, CDCl3) δ 8.63-8.54 (m, 1H), 8.45 (dd, J = 8.7, 0.8 Hz, 1H), 8.02 |
| | | | | (dd, J = 8.7, 2.4 Hz, 1H), 7.60-7.33 (m, 4H), 7.28 (s, 1H), 7.19 (s, 1H), 6.82 |
| | | | | (d, J = 8.6 Hz, 1H), 4.68-4.45 (m, 4H), 3.62 (ddd, J = 15.4, 9.0, 6.4 Hz, 1H). |
| 445 | C | 3.35 | 430.2 | ¹H NMR (400 MHz, Methanol-d₄) δ 8.75-8.60 (m, 2H), 8.02 (dd, J = 8.8, 2.4 Hz, |
| | | | | 1H), 7.64 (dd, J = 8.1, 1.1 Hz, 2H), 7.56 (dd, J = 9.2, 7.0 Hz, 1H), 7.09 (dd, J = |
| | | | | 8.8, 0.8 Hz, 1H), 4.22 (s, 3H). |
| 446 | C | 3.62 | 483.2 | ¹H NMR (400 MHz, Methanol-d₄) δ 8.38-8.29 (m, 2H), 8.25 (s, 1H), 7.76 (dd, J = |
| | | | | 8.8, 0.7 Hz, 1H), 7.73-7.61 (m, 3H), 7.56 (dd, J = 9.2, 7.0 Hz, 1H). |
| 447 | B | 3.182 | 500 | — |
| 448 | A | 2.668 | 488.2 | 1H NMR (600 MHz, cdcl3) δ 9.08 (s, 1H), 8.57 (s, 1H), 8.32 (s, 1H), 7.60 (d, J = |
| | | | | 8.6 Hz, 1H), 7.48 (d, J = 8.3 Hz, 2H), 7.39 (dd, J = 8.8, 7.5 Hz, 1H), 6.86-6.68 |
| | | | | (m, 2H), 5.51 (s, 1H), 3.69 (dd, J = 87.7, 30.2 Hz, 4H), 2.78 (s, 1H), 2.60 (s, |
| | | | | 3H), 2.37 (d, J = 23.7 Hz, 3H), 2.01 (d, J = 24.7 Hz, 2H). |
| 449 | A | 3.622 | 449.2 | |
| 450 | C | 3.169 | 437.20 | ¹H NMR (400 MHz, Chloroform-d) δ 8.35 (s, 1H), 7.97 (d, J = 3.0 Hz, 1H), 7.72- |
| | | | | 7.67 (m, 1H), 7.65-7.49 (m, 4H), 7.08-7.03 (m, 2H), 6.87 (s, 1H), 5.50 (s, 1H), |
| | | | | 5.45-5.08 (m, 1H), 4.02-3.61 (m, 5H), 2.18 (d, J = 98.0 Hz, 2H). |
| 451 | C | 3.246 | 508.00 | 1H NMR (400 MHz, Chloroform-d) d 8.51 (s, 1H), 7.69 (s, 1H), 7.56-7.50 (m, 2H), |
| | | | | 7.48-7.39 (m, 3H), 7.08 7.02 (m, 2H), 6.79 (s, 1H), 5.49 (s, 1H), 4.14 (t, J = |
| | | | | 5.1 Hz, 4H), 3.09 (s, 4H). |
| 452 | C | 3.246 | 483.20 | ¹H NMR (400 MHz, Chloroform-d) δ 8.40 (s, 1H), 7.93 (d, J = 2.8 Hz, 1H), 7.68- |
| | | | | 7.63 (m, 1H), 7.59-7.46 (m, 2H), 7.45-7.37 (m, 2H), 7.08-6.99 (m, 2H), 6.84 (s, |
| | | | | 1H), 5.52 (s, 1H), 4.17-3.97 (m, 4H), 3.05 (t, J = 5.4 Hz, 4H). |
| 453 | E | 0.834 | 473.2 | |
| 454 | C | 3.031 | 435.20 | 1H NMR (400 MHz, Chloroform-d) d 8.29 (s, 1H), 7.92 (d, J = 3.0 Hz, 1H), 7.68- |
| | | | | 7.62 (m, 1H), 7.58-7.44 (m, 2H), 7.43-7.35 (m, 2H), 7.06-6.98 (m, 2H), 6.82 (s, |
| | | | | 1H), 5.45 (s, 1H), 3.66 (d, J = 17.2 Hz, 6H). |
| 455 | B | 2.485 | 495.18 | 1H NMR (400 MHz, Methanol-d4) δ 8.28 (d, J = 2.2 Hz, 1H), 8.10 (s, 1H), 7.63 (dd, |
| | | | | J = 8.1, 1.1 Hz, 2H), 7.55 (dd, J = 9.2, 7.0 Hz, 1H), 7.50 (dd, J = 8.5, 2.8 Hz, |
| | | | | 1H), 7.42 (dd, J = 8.5, 0.7 Hz, 1H), 3.73 (s, 2H), 3.16-2.96 (m, 8H). |
| 456 | C | 3.75 | 458.2 | 1H NMR (400 MHz, Methanol-d₄) δ 8.12 (s, 1H), 7.68-7.48 (m, 5H), 7.05 (d, J = 8.8 |
| | | | | Hz, 2H), 4.10 (s, 2H), 3.83 (s, 2H), 1.31 (s, 6H). |
| 457 | C | 3.030 | 499.20 | 1H NMR (400 MHz, Methanol-d4) d 8.27 (s, 1H), 7.96 (dd, J = 8.3, 1.4 Hz, 1H), |
| | | | | 7.91 (dd, J = 7.8, 1.3 Hz, 1H), 7.69 (t, J = 8.1 Hz, 1H), 7.48-7.40 (m, 2H), 7.14- |
| | | | | 7.06 (m, 2H), 4.05 (d, J = 6.0 Hz, 4H), 3.17 (t, J = 5.5 Hz, 4H) |
| 458 | C | 3.094 | 451.20 | 1H NMR (400 MHz, Methanol-d4) d 8.26 (s, 1H), 7.96 (dd, J = 8.2, 1.4 Hz, 1H), |
| | | | | 7.91 (dd, J = 7.8, 1.4 Hz, 1H), 7.69 (t, J = 8.1 Hz, 1H), 7.43-7.31 (m, 2H), 7.12- |
| | | | | 7.04 (m, 2H), 3.64 (d, J = 14.6 Hz, 8H). |
| 459 | C | 3.234 | 453.20 | |
| 460 | C | 3.8 | 480.2 | 1H NMR (400 MHz, Methanol-d₄) δ 8.35 (dd, J = 2.9, 0.7 Hz, 1H), 8.26 (s, 1H), |
| | | | | 7.69-7.54 (m, 5H), 2.24 (t, J = 19.0 Hz, 3H). |
| 461 | C | 2.958 | 464.2 | 1H NMR (600 MHz, cdcl3) δ 7.46 (d, J = 8.4 Hz, 2H), 7.43 (d, J = 0.9 Hz, 1H), |
| | | | | 7.39-7.36 (m, 1H), 7.35 (d, J = 11.0 Hz, 2H), 7.21 (s, 1H), 6.67 (s, 1H), 5.34 |
| | | | | (s, 1H), 4.91 (s, 2H), 3.69-3.63 (m, 2H), 3.60 (q, J = 4.6 Hz, 4H), 3.55 (q, J = |
| | | | | 4.5, 4.1 Hz, 2H). |
| 462 | C | 3.183 | 409.2 | |
| 463 | B | 3.05 | 509.2 | ¹H NMR (600 MHz, Methanol-d₄) δ 8.34 (d, J = 2.8 Hz, 1H), 8.22 (s, 1H), 7.70 (d, |
| | | | | J = 8.6 Hz, 1H), 7.66-7.61 (m, 2H), 7.56 (dd, J = 8.9, 7.4 Hz, 1H), 7.52 (dd, J = |
| | | | | 8.6, 2.9 Hz, 1H), 4.18 (s, 4H), 3.26 (s, 4H). |
| 464 | C | 3.697 | 459.20 | ¹H NMR (400 MHz, Methanol-d₄) δ 8.35 (d, J = 2.9 Hz, 1H), 8.23 (s, 1H), 7.94 (d, |
| | | | | J = 8.7 Hz, 1H), 7.72-7.62 (m, 2H), 7.58 (dd, J = 9.2, 6.9 Hz, 1H), 7.49 (dd, |
| | | | | J = 8.7, 2.9 Hz, 1H), 4.39 (d, J = 1.1 Hz, 2H), 3.86 (d, J = 1.0 Hz, 2H), 1.34 (s, 6H). |
| 465 | C | 3.796 | 459.20 | ¹H NMR (400 MHz, Methanol-d₄) δ 8.64 (s, 1H), 8.58 (dd, J = 2.4, 0.8 Hz, 1H), |
| | | | | 7.89 (dd, J = 8.7, 2.4 Hz, 1H), 7.71-7.62 (m, 2H), 7.62-7.50 (m, 1H), 6.93 (dd, |
| | | | | J = 8.8, 0.8 Hz, 1H), 4.15 (s, 2H), 3.86 (s, 2H), 1.33 (s, 6H). |
| 466 | C | 3.092 | 521.2 | 1H NMR (400 MHz, Methanol-d4) δ 8.14 (s, 1H), 7.67-7.62 (m, 2H), 7.57 (dd, J = |
| | | | | 9.2, 7.0 Hz, 1H), 7.51-7.44 (m, 2H), 7.15-7.06 (m, 2H), 4.29-4.12 (m, 2H), 3.88 |
| | | | | (ddd, J = 14.5, 8.6, 3.2 Hz, 2H), 3.31-3.20 (m, 4H), 2.81 (s, 3H). |
| 467 | C | 3.670 | 476.2 | 1H NMR (400 MHz, Methanol-d4) δ 8.13 (s, 1H), 7.68-7.62 (m, 2H), 7.57 (dd, J = |
| | | | | 9.2, 6.9 Hz, 1H), 7.45-7.32 (m, 2H), 7.15-7.06 (m, 2H), 3.89 (s, 4H), 2.68 (d, |
| | | | | J = 5.8 Hz, 4H) |
| 468 | C | 2.97 | 507.2 | 1H NMR (400 MHz, Methanol-d4) δ 8.14 (s, 1H), 7.67-7.62 (m, 2H), 7.57 (dd, J = |
| | | | | 9.2, 7.0 Hz, 1H), 7.50-7.44 (m, 2H), 7.14-7.08 (m, 2H), 4.27-4.12 (m, 2H), 3.94 |
| | | | | (ddd, J = 14.5, 8.3, 3.2 Hz, 2H), 3.25 (d, J = 13.3 Hz, 4H). |
| 469 | C | 2.921 | 512.0 | 1H NMR (400 MHz, CDCl3) δ 7.54-7.38 (m, 5H), 7.36 (s, 1H), 7.28 (s, 1H), 6.72 (s, |
| | | | | 1H), 5.43 (s, 1H), 4.98 (s, 2H), 4.25-4.04 (m, 4H), 2.98 (d, J = 35.0 Hz, 4H). |
| 470 | B | 3.326 | 494.2 | |
| 471 | B | 3.338 | 533 | |
| 472 | E | 0.92 | 534.0 | |
| 473 | HPLC | 3.47 | 478.2 | |
| | MS-C | | | |
| 474 | D | 3.11 | 480.2 | |
| 475 | A | 3.59 | 485.2 | |
| 476 | B | 3.16 | 490.2 | |
| 477 | D | 3.12 | 497.2 | |
| 478 | | 1.75 | 473.1 | ¹H NMR (CD₃OD) δ 3.51 (m, 1H), δ 4.25 (m, 2H), δ 4.52 (d, 2H), δ 7.06 (d, 2H), δ |
| | | | | 7.55 (m, 1H), δ 7.63 (m, 4H), δ 8.14 (s, 1H). |

### Example 20

The compounds listed in Table 1 were tested for their ability to inhibit a set of kinases according to the following procedures.

Biochemical Assays:

The basic protocol for TR-FRET LanthaScreen Eu Kinase Binding Assay inhibitor studies were performed as follows. LanthaScreen Kinase Binding Assays (ThermoFisher/USA) to evaluate inhibitors were performed by addition of 5 μL of test compound in corresponding DMSO dilutions/5 μl of kinase/antibody mixture, 5 μL of tracer into 384 well small volume plates. After incubation for 1 hour at room temperature, plates were read. Data analysis of emission ratios was according LanthaScreen Eu Kinase Binding Assay protocol.

Kinase and assay components were adjusted to final concentrations according to kit protocol. For LRRK2: 5 nM wt human LRRK2, catalytic site, or G2019S human LRRK2, catalytic site (ThermoFisher/USA), 2 nM Eu-Anti-GST Antibody, 10 nM Kinase Tracer 236 in 1× Kinase Buffer A. For NUAK1: 8 nM wt human NUAK1, full length (ThermoFisher/USA), 2 nM Eu-Anti-His Antibody, 5 nM Kinase Tracer 236 in 1× Kinase Buffer A.

Basic protocol for Z′-LYTE Assays (ThermoFisher/USA) inhibitor studies involved two steps:
Kinase reaction: Addition of 2 μL of test compound in corresponding DMSO dilutions, 5 L of kinase/substrate mixture, 3 μL of ATP into 384 well small volume plates. Incubation for at least 1 hour at room temperature.
Development reaction: Addition of 5 μL development reagent, incubation for 1 hour at room temperature, addition of 5 μL stop reagent, read plate. Data analysis of emission ratios according to Z′-LYTE assay protocol.

Kinase and assay components were adjusted to final concentrations according to kit protocol. For JAK2: 5 nM wt human JAK2, catalytic site (ThermoFisher/USA), 2 μM Z′-LYTE Tyr 06 Peptide, 36 μM ATP in Kinase Buffer.

Basic protocol for HTRF KinEASE assay (Cisbio/FRA) inhibitor studies involved two steps:
Enzymatic step: Addition of 4 μL of test compound in corresponding DMSO dilutions, 4 μL of kinase/substrate mixture, 2 μL of ATP into 384 well small volume plates. Incubation for at least 30 minutes at room temperature.
Detection step: Addition of 5 μL antibody and 5 μL streptavidin-XL665, read plate after 60 minutes. Data analysis of emission ratios according KinEASE assay protocol.

Kinase and assay components were adjusted to final concentrations according to kit protocol. For TYK2: 2 nM wt human TYK2, catalytic site (SignalChem/CAN), 1 μM HTRF KinEASE-TK Substrate-biotin, 1 μM ATP in 1× Kinase Buffer. For JAK1: 4 nM wt human JAK1, catalytic site (ThermoFisher/USA), 1 μM HTRF KinEASE-TK Substrate-biotin, 2.5 μM ATP in 1× Kinase Buffer. For JAK2: 1 nM wt human JAK2, catalytic site (SignalChem/CAN), 1 μM HTRF KinEASE-TK Substrate-biotin, 100 μM ATP in 1× Kinase Buffer.

Results for from biochemical tests were as follows:

### Activity Toward TYK2

The following compounds had an IC₅₀ lower than 10 nM: Compound Nos. 17, 22, 32, 36, 42, 45, 46, 57, 58, 62, 83, 84, 85, 86, 87, 88, 89, 91, 95, 203, 207, 212, 214, 215, 227, 230, 231, 237, 238, 243, 247, 249, 254, 259, 275, 276, 277, 281, 283, 292, 294, 297, 299, 301, 302, 305, 314, 315, 316, 332, 333, 338, 342, 347, 348, 349, 361, 371, 411, 413, 414, 415, 416, 425, 426, 450, 451, 452, 453, 454, 456, 457, 458, 459, 463, 466, 467, and 468.

The following compounds had an IC₅₀ between 10 nM and 100 nM: Compound Nos. 12, 13, 14, 15, 16, 26, 27, 28, 29, 30, 31, 34, 35, 38, 40, 44, 47, 48, 49, 50, 51, 52, 54, 55, 56, 60, 61, 63, 68, 69, 70, 72, 73, 74, 78, 79, 80, 82, 90, 92, 93, 94, 96, 202, 204, 205, 206, 208, 209, 210, 211, 213, 216, 225, 226, 228, 229, 232, 233, 241, 242, 246, 248, 251, 267, 268, 269, 270, 271, 273, 284, 285, 286, 288, 291, 293, 295, 296, 298, 300, 303, 304, 306, 307, 308, 310, 313, 317, 318, 319, 321, 322, 323, 325, 326, 327, 330, 331, 340, 341, 351, 354, 355, 356, 357, 358, 359, 360, 362, 363, 364, 365, 366, 367, 370, 373, 375, 376, 378, 383, 385, 386, 387, 388, 391, 400, 412, 417, 418, 419, 421, 422, 423, 424, 429, 433, 435, 436, 438, 443, 444, 445, 447, 455, 461, 464, 469, 472, and 473.

The following compounds had an IC₅₀ between 100 nM and 1000 nM: Compound Nos. 4, 5, 6, 7, 8, 10, 11, 18, 21, 23, 24, 25, 33, 37, 39, 41, 43, 53, 59, 64, 67, 71, 75, 77, 81, 201, 218, 224, 236, 240, 253, 257, 260, 261, 263, 266, 272, 274, 278, 279, 280, 287, 289, 309, 311, 324, 328, 329, 334, 337, 339, 343, 344, 345, 346, 350, 352, 368, 369, 372, 374, 377, 379, 380, 381, 383, 384, 390, 392, 393, 394, 395, 397, 398, 401, 402, 403, 405, 407, 420, 427, 428, 430, 431, 432, 434, 439, 440, 441, 442, 448, 449, 460, 462, 465, 470, 471, 475, and 476.

### Activity Toward LRRK2(Wt) and LRRK2(G2019)

The following compounds had an IC₅₀ between 10 nM and 100 nM: Compound Nos. 12, 35, 36, 45, 56, 58, 62, 63, 208, 211, 304, and 309.

The following compounds had an IC₅₀ between 100 nM and 1000 nM: Compound Nos. 4, 7, 8, 10, 11, 13, 14, 15, 16, 17, 18, 22, 23, 26, 27, 30, 31, 32, 34, 37, 38, 40, 42, 46, 47, 48, 49, 50, 51, 52, 53, 54, 57, 59, 60, 61, 64, 68, 70, 71, 80, 82, 83, 84, 85, 86, 87, 88, 89, 202, 205, 206, 207, 212, 225, 227, 228, 229, 230, 231, 232, 233, 246, 247, 248, 249, 259, 260, 267, 268, 272, 276, 283, 284, 285, 286, 287, 288, 295, 296, 299, 302, 303, 305, 308, 310, 313, 315, 316, 317, 318, 321, 322, 323, 326, 331, 333, 342, 344, 347, 349, 355, 359, 361, 376, 386, 387, 388, 391, 394, 395, 400, 414, 415, 416, 417, 418, 421, 422, 423, 425, 426, 436, 450, 452, 453, 454, 456, 466, and 472.

### Activity Toward NUAK1

The following compounds had an IC₅₀ between 10 nM and 100 nM: Compound Nos. 8, 13, 14, 22, 38, 227, 287, 288, and 295.

The following compounds had an IC₅₀ between 100 nM and 1000 nM: Compound Nos. 4, 7, 12, 15, 16, 27, 30, 31, 35, 36, 44, 48, 54, 56, 58, 61, 62, 68, 82, 86, 87, 88, 91, 202, 203, 205, 206, 207, 208, 211, 212, 214, 225, 228, 229, 230, 232, 237, 248, 249, 251, 284, 285, 286, 296, 308, 309, 313, 314, 317, 318, 319, 323, 327, 331, 332, 333, 338, 342, 349, 359, 361, 403, 407, 416, 417, 422, 423, 424, 452, 453, and 454.

### Activity Toward JAK1

The following compounds had an IC₅₀ lower than 10 nM: Compound Nos. 42, 46, 229, 231, 241, 291, 436, 451, 452, 453, 467, and 468.

The following compounds had an IC₅₀ between 10 nM and 100 nM: Compound Nos. 17, 22, 26, 31, 32, 34, 35, 36, 38, 39, 40, 49, 51, 53, 58, 59, 60, 74, 80, 82, 84, 85, 86, 87, 88, 91, 92, 94, 96, 203, 205, 206, 207, 208, 212, 214, 215, 216, 225, 227, 228, 230, 232, 233, 238, 242, 243, 246, 247, 248, 254, 259, 260, 266, 267, 268, 269, 270, 271, 272, 273, 275, 276, 285, 292, 293, 294, 295, 302, 303, 305, 308, 310, 313, 314, 315, 316, 317, 318, 319, 321, 322, 326, 327, 330, 333, 347, 348, 359, 361, 365, 376, 377, 386, 387, 388, 391, 411, 412, 413, 414, 415, 416, 417, 418, 419, 421, 422, 423, 425, 426, 431, 432, 433, 435, 439, 440, 443, 444, 445, 447, 448, 450, 454, 456, 457, 458, 459, 461, 462, 463, 466, 469, 472, 473, and 476.

The following compounds had an IC₅₀ between 100 nM and 1000 nM: Compound Nos. 8, 11, 12, 13, 14, 15, 16, 18, 27, 28, 29, 30, 33, 37, 41, 43, 44, 45, 47, 48, 50, 52, 54, 55, 56, 57, 61, 62, 63, 64, 67, 68, 69, 70, 71, 72, 73, 75, 76, 77, 78, 79, 83, 89, 90, 93, 95, 202, 204, 209, 210, 211, 213, 217, 218, 222, 224, 226, 234, 237, 240, 249, 251, 253, 261, 263, 277, 281, 283, 284, 286, 287, 288, 296, 297, 298, 299, 300, 304, 307, 309, 323, 324, 325, 331, 332, 334, 338, 340, 341, 342, 349, 350, 351, 352, 354, 355, 356, 357, 358, 360, 362, 363, 364, 366, 367, 370, 371, 373, 374, 375, 378, 379, 380, 381, 383, 383, 384, 385, 390, 392, 393, 394, 395, 397, 398, 400, 401, 402, 405, 407, 420, 424, 428, 429, 430, 437, 438, 441, 442, 449, 455, 460, 464, 470, 471, and 475.

### Activity Toward JAK2

The following compounds had an IC₅₀ between 100 nM and 1000 nM: Compound Nos. 12, 13, 17, 22, 32, 36, 57, 58, 62, 87, 88, 90, 94, 96, 202, 203, 205, 206, 207, 210, 211, 212, 227, 229, 238, 322, 333, 347, 414, 416, 425, 451, 452, 454, and 457.

### Example 21

Certain compounds that were used as reagents in synthesis schemes described herein were prepared by the following schemes.

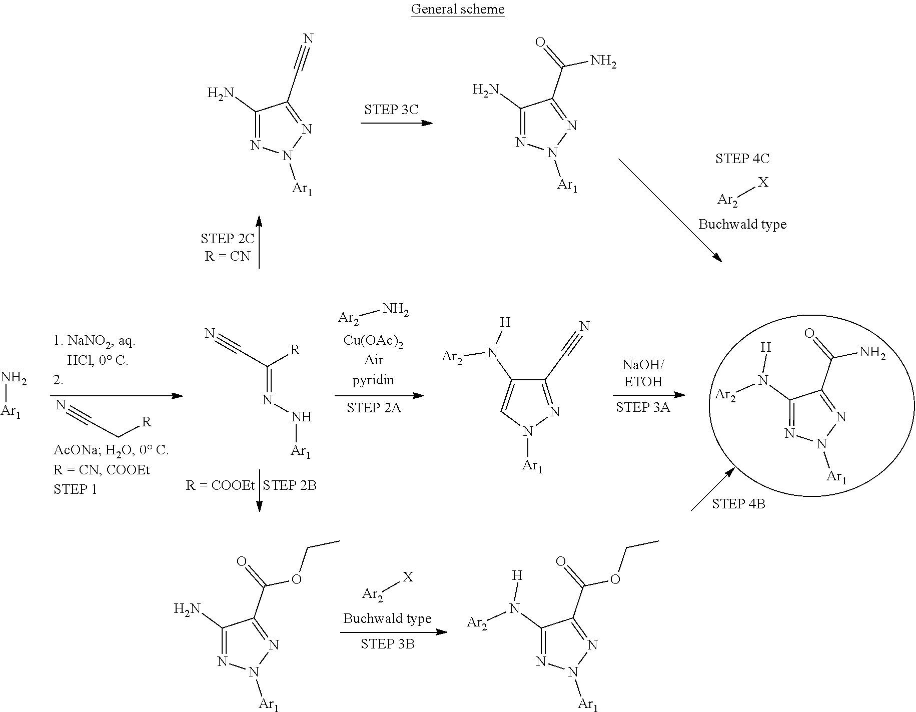

### General Procedure:

Step 1:

In a round bottom flask, equipped with a magnetic stirrer, aniline (1 eq) was dissolved in aq. 6M HCl (1 mL/mmol). The reaction was cooled to −10° C. in an ice-salt bath. Aq. 1M NaNO₂ (1 mL/mmol) was added with a dropping funnel in 20 min at −10° C. The reaction was stirred at −10° C. for 90 min and then added at once to a solution of the Malone component (1.5 eq) and NaOAc (1 eq.) in MeOH (139 mL) kept at −10° C. in an ice-salt bath. The reaction was stirred at −10° C. until a precipitate formed. The solid was collected by suction filtration. The resulting solution was kept overnight at 5° C. and the filtration repeated. After concentration under reduced pressure and cooling overnight at 5° C., the filtration was repeated. All the solids from the filtrations were assembled and dried in vacuum.

The desired products were obtained as yellow solid for R=CN or red solids for R=COOEt.

### Route A:

Step 2A:

Product of step 1 and the amine (1.5 eq.) are dissolved in pyridine (0.2 mL/mmol). The mixture is stirred at 80° C. After 20 min, Cu(OAc)₂ (1 eq.) is added. Then the mixture is stirred at 80° C. overnight. The mixture is evaporated, then the residue is dissolved in (5-10 mL/mmol) acetonitrile and add cold water (˜50-100 mL/mmol). The precipitated crude product is collected by filtration and purified by gradient flash chromatography.

Step 3A:

The product of step 2A is dissolved in water/ethanol 1:1 (1.5 mL/mmol) and 5 eq. NaOH (5 eq.) was added. The mixture is stirred at rt overnight. The mixture is evaporated, dissolve in water and extracted with DCM. The organic layer is dried over Na₂SO₄, filtered and concentrated under reduced pressure to get the crude product. The crude product is purified by semi preparative HPLC (acetonitrile/water). After evaporation and Lyophilization a colourless solid compound is obtained

### Route B

Step 2B:

In a 20 mL microwave vial, equipped with a magnetic stirrer, product of Step1 (1 eq) was dissolved in 24 mL/g of dry Pyridine. NH₄Cl (1.3 eq) was added. The reaction was stirred at 80° C. for 20 min. Cu(OAc)2H2O (2 eq.) was added and the reaction was stirred at 80° C. overnight and monitored by HPLC MS. After completion, the solvent was removed under reduced pressure. The residue was suspended in EtOAc and filtered through a celite pad. The filtrate was concentrated under reduced pressure till dryness and the final product purified by gradient silica gel column chromatography (EtOAc:Cyc, 0 to 100%). The desired product was obtained as yellow solid

Step 3B:

The product of step 2B, bromo-aryl/heteroaryl compound (1.2 eq.), XPhos (0.25 eq.), 3rd Generation t-BuBrett Pd precatalyst (0.25 eq.), and t-BuONa (1.5 eq.) were mixed in a microwave vial. The vial was sealed and purged with nitrogen. Then, anhydrous 1,4-dioxane (3 mL) was added to the mixture. The mixture was bubbled with N₂ for 15 min, then it was stirred at 95° C. for 1 to 3 h. The reaction was controlled by LC-MS. When the reaction was finished, the crude was cooled down till room temperature, filtered through a HPLC filter using abundant methanol, and then concentrated until dryness under reduced pressure. The compound was purified by gradient silica gel column chromatography

Step 4B:

The product of step 3B (1 eq.) was dissolved in THF (5 mL/1 mmol) and then ammonium hydroxide solution was added (33% in water, 30 mL/mmol). The reaction was stirred at room temperature overnight. After completion, the product was extracted from the water using ethyl acetate (3×100 mL). The combined organic layers were dried over Na₂SO₄, filtered and concentrated under reduced pressure. The product was then purified by preparative reversed phase HPLC chromatography. After that purification, the tubes containing the product were combined, dried under reduced pressure, re-dissolved in t-Butanol/water, freeze and lyophilized.

### Route C

Step 2C:

In a 20 mL microwave vial, equipped with a magnetic stirrer, product of Step1 (1 eq) was dissolved in 24 mL/g of dry Pyridine. NH₄Cl (1.3 eq) was added. The reaction was stirred at 80° C. for 20 min. Cu(OAc)2H2O (2 eq.) was added and the reaction was stirred at 80° C. overnight and monitored by HPLC MS. After completion, the solvent was removed under reduced pressure. The residue was suspended in EtOAc and filtered through a celite pad. The filtrate was concentrated under reduced pressure till dryness and the final product purified by gradient silica gel column chromatography (EtOAc:Cyc-hexane, 0 to 100%).

The desired product was obtained as a yellow solid

Step 3C:

In a round bottom flask equipped with a magnetic stirrer, the product of Step 2C (1 eq) was dissolved in EtOH:H2O, (1:1). NaOH (5 eq) was added and the reaction was stirred at 40° C. overnight and monitored by HPLC MS. After completion, the solvent was removed under reduced pressure. The residue was partitioned between DCM-H2O (1:1) and extracted with DCM (3×). The organic layers were assembled, washed with brine and dried (MgSO₄). The solvent was removed under reduced pressure to afford the desired product as a yellow solid.

Step 4C:

In a 20 mL microwave vial, equipped with a magnetic stirrer, the product of Step3C (1 eq), the bromide (1.5 eq.) and NaOtBu (3 eq) were dissolved in 2 mL of dry dioxane. The resulting solution was degassed for 5 min in an ultrasound bath. tBuBrettphos Pd G3 (0.25 eq) and XPhos (0.25 eq) were added and the reaction further degassed as before. The reaction was stirred at 100° C. in an oil bath and monitored by HPLC MS. After completion, the solution was cooled down to room temperature, diluted with MeOH and filtered through a celite pad. The resulting solution was concentrated under reduced pressure and the product pre purified on an by gradient silica gel column chromatography. The final product was further purified using an Agilent semi preparative reverse phase machine. The product was obtained as a white powder after lyophilization from tBuOH—H2O.

### Route D

The intermediate from Step 2C was treated as described in Step 4C to produce the corresponding nitrile from Step 2A, which was then hydrolyze according to Step 3A.

### Example 22

Chemical analytical data for the compounds listed in Table 1a is summarized in Table 8.

TABLE 8

| | | HPLC- | | | |
|---|---|---|---|---|---|
| Compound | Synthesis | MS | retention | m + H | |
| No | Route | Method | time (min) | Found | NMR |
| 101 | A | C | 3.450 | 461.2 | 1H NMR (400 MHz, Chloroform-d) δ 8.64 (s, 1H), 7.59 (d, J = 8.4 Hz, 2H), 7.56-7.46 |
| | | | | | (m, 3H), 7.43 (d, J = 8.5 Hz, 2H), 6.68 (s, 1H), 5.80-5.60 (m, 1H), 3.71 (s, 8H). |
| 102 | A | C | 3.080 | 474.2 | 1H NMR (400 MHz, Chloroform-d) δ 8.65 (s, 1H), 7.64-7.56 (m, 2H), 7.58-7.50 (m, 2H), |
| | | | | | 7.48 (dd, J = 9.4, 6.5 Hz, 1H), 7.47-7.39 (m, 2H), 6.67 (s, 1H), 5.74 (s, 1H), 3.83 |
| | | | | | (s, 4H), 2.67 (s, 4H), 2.49 (s, 3H). |
| 103 | A | C | 2.980 | 416.2 | |
| 104 | A | C | 3.580 | 430.2 | |
| 105 | A | C | 3.020 | 488.2 | |
| 106 | A | C | 4.000 | 463.0 | |
| 107 | A | C | 3.440 | 456.2 | 1H NMR (800 MHz, Chloroform-d) δ 8.74 (s, 1H), 7.65 (s, 1H), 7.66-7.60 (m, 3H), 7.62- |
| | | | | | 7.53 (m, 2H), 7.54-7.46 (m, 1H), 6.66 (s, 1H), 5.61 (s, 1H), 4.57-4.53 (m, 2H), 4.50 |
| | | | | | (s, 2H), 3.56 (ddt, J = 5.5, 9.0, 5.6 Hz, 1H). |
| 108 | A | C | 3.040 | 474.2 | |
| 109 | A | C | 3.430 | 469.2 | |
| 110 | A | C | 3.820 | 470.2 | |
| 111 | A | C | 3.870 | 470.2 | |
| 112 | C | B | 3.731 | 526.2 | 1H NMR (600 MHz, Methanol-d4) d 7.75 (d, J = 1.4 Hz, 1H), 7.72-7.69 (m, 2H), 7.67- |
| | | | | | 7.65 (m, 2H), 7.63-7.60 (m, 2H), 7.59 (s, 1H), 4.15 (t, J = 5.3 Hz, 2H), 3.80 (t, J = |
| | | | | | 5.3 Hz, 2H). |
| 113 | C | B | 3.317 | 479.2 | 1H NMR (400 MHz, Methanol-d4) d 8.60 (s, 1H), 7.77-7.72 (m, 2H), 7.66-7.58 (dd, J = |
| | | | | | 2.7. 25 Hz, 1H), 7.64-7.60 (m, 2H), 7.58-7.54 (m, 2H), 6.16 (tt, J = 54.3, 2.8 Hz, |
| | | | | | 1H), 4.56 (td, J = 15.6, 2.9 Hz, 3H). |
| 114 | C | C | 3.170 | 461.2 | 1H NMR (400 MHz, Methanol-d4) δ 7.70-7.51 (m, 3H), 7.51-7.37 (m, 2H), 6.97-6.81 (m, |
| | | | | | 2H), 4.42 (brs, 1H), 2.99 (brs, 2H), 2.74 (brs, 2H), 2.54 (s, 3H), 2.13-1.76 (m, 4H). |
| 115 | C | C | 4.080 | 496.2 | 1H NMR (400 MHz, Methanol-d4) δ 7.76-7.52 (m, 8H), 3.78 (s, 3H). |
| 116 | B | B | 3.337 | 457.2 | |
| 117 | C | B | 3.061 | 429.2 | 1H NMR (400 MHz, Chloroform-d) δ 8.64 (s, 1H), 7.59 (d, J = 8.4 Hz, 2H), 7.56-7.46 |
| | | | | | (m, 3H), 7.43 (d, J = 8.5 Hz, 2H), 6.68 (s, 1H), 5.80-5.60 (m, 1H), 3.71 (s, 8H). |
| 118 | | C | 4.140 | 540.2 | 1H NMR (400 MHz, Chloroform-d) δ 8.66 (s, 1H), 7.69-7.62 (m, 2H), 7.60-7.51 (m, 4H), |
| | | | | | 7.53-7.44 (m, 2H), 6.66 (s, 1H), 5.64 (s, 1H), 4.17 (t, J = 5.1 Hz, 2H), 3.65 (t, J = |
| | | | | | 5.1 Hz, 2H), 3.37 (s, 3H). |
| 119 | A | C | 3.502 | 463.2 | 1H NMR (400 MHz, Chloroform-d) δ 8.68 (s, 1H), 7.76 (d, J = 8.7 Hz, 2H), 7.63-7.51 |
| | | | | | (m, 4H), 7.48 (ddd, J = 9.3, 6.5, 0.7 Hz, 1H), 6.80 (d, J = 5.4 Hz, 1H), 6.66 (s, |
| | | | | | 1H), 5.62 (s, 1H), 3.59 (q, J = 5.9 Hz, 4H), 3.40 (d, J = 0.7 Hz, 3H), 1.90 (p, J = |
| | | | | | 5.8 Hz, 2H). |
| 120 | A | C | 3.250 | 447.2 | 1H NMR (400 MHz, Chloroform-d) δ 8.27 (s, 1H), 7.58-7.39 (m, 5H), 6.90 (d, J = 8.8 |
| | | | | | Hz, 2H), 6.63 (s, 1H), 6.09 (broad s, NH), 5.76 (s, 1H), 4.46 (s, 1H), 3.27 (s, 2H), |
| | | | | | 3.03 (s, 2H), 2.13 (d, J = 14.8 Hz, 2H), 2.00-1.86 (m, 2H). |
| 121 | B | C | 4.399 | 511.2 | ¹H NMR (400 MHz, Chloroform-d) δ 8.70 (s, 1H), 7.73-7.63 (m, 2H), 7.55-7.46 (m, 2H), |
| | | | | | 7.45 (dd, J = 9.4, 6.5 Hz, 1H), 7.39-7.30 (m, 2H), 6.63 (s, 1H), 5.62 (s, 1H), 2.79 |
| | | | | | (q, J = 7.6 Hz, 2H), 1.30 (t, J = 7.6 Hz, 3H). |
| 122 | A | C | 3.020 | 502.3 | 1H NMR (400 MHz, Chloroform-d) δ 8.62 (s, 1H), 7.65-7.45 (m, 5H), 7.45-7.37 (m, 2H), |
| | | | | | 6.66 (s, 1H), 5.68 (s, 1H), 3.20 (s, 2H), 2.91 (s, 2H), 2.56 (td, J = 11.4, 5.8 Hz, |
| | | | | | 1H), 2.36 (s, 6H), 1.90 (s, 2H), 1.51 (d, J = 12.5 Hz, 2H). |
| 123 | A | C | 3.410 | 449.2 | 1H NMR (400 MHz, Chloroform-d) δ 8.68 (s, 1H), 7.83-7.74 (m, 2H), 7.65-7.57 (m, 2H), |
| | | | | | 7.58-7.44 (m, 3H), 6.65 (s, 1H), 6.48 (d, J = 5.3 Hz, 1H), 5.62 (s, 1H), 3.67 (td, |
| | | | | | J = 5.6, 4.3 Hz, 2H), 3.58 (dd, J = 5.4, 4.2 Hz, 2H), 3.41 (s, 3H). |
| 124 | A | C | 3.150 | 447.2 | 1H NMR (400 MHz, Methanol-d4) δ 7.72-7.59 (m, 7H), 4.68-4.59 (m, 2H), 4.40 (s, 1H), |
| | | | | | 4.20 (s, 1H), 3.95 (d, J = 10.5 Hz, 1H). |
| 125 | B | C | 3.470 | 457.2 | ¹H NMR (400 MHz, Methanol-d₄) δ 7.82-7.74 (m, 2H), 7.70-7.60 (m, 3H), 7.59-7.51 (m, |
| | | | | | 3H), 4.58 (sept, J = 6.8 Hz, 1H), 1.50 (d, J = 6.7 Hz, 6H). |
| 126 | B | C | 3.850 | 443.2 | ¹H NMR (400 MHz, Methanol-d₄) δ 7.85-7.72 (m, 2H), 7.72-7.54 (m, 4H), 7.49-7.36 (m, |
| | | | | | 2H), 2.72 (q, J = 7.5 Hz, 2H), 1.22 (t, J = 7.5 Hz, 3H). |
| 127 | C | B | 2.419 | 475.2 | 1H NMR (600 MHz, cdcl3) δ 9.22 (s, 1H), 8.40 (t, J = 2.5 Hz, 1H), 7.97 (dd, J = 8.6, |
| | | | | | 0.9 Hz, 1H), 7.75 (dd, J = 8.7, 2.3 Hz, 1H), 7.53 (d, J = 8.6 Hz, 2H), 7.48 (dd, J = |
| | | | | | 9.8, 2.7 Hz, 1H), 6.64 (s, 1H), 5.82 (s, 1H), 4.15-3.29 (m, 8H), 2.35 (s, 3H). |
| 128 | A | C | 3.460 | 437.2 | 1H NMR (400 MHz, Chloroform-d) δ 8.71 (s, 1H), 7.83-7.73 (m, 2H), 7.65-7.57 (m, 2H), |
| | | | | | 7.55 (d, J = 6.8 Hz, 2H), 7.49 (dd, J = 9.4, 6.5 Hz, 1H), 6.66 (s, 1H), 6.43 (t, J = |
| | | | | | 5.9 Hz, 1H), 5.63 (s, 1H), 4.68 (t, J = 4.7 Hz, 1H), 4.56 (t, J = 4.7 Hz, 1H), 3.83 |
| | | | | | (q, J = 5.1 Hz, 1H), 3.76 (q, J = 5.1 Hz, 1H). |
| 129 | A | C | 3.560 | 449.0 | 1H NMR (400 MHz, Chloroform-d) δ 8.71 (s, 1H), 7.74-7.58 (m, 4H), 7.58-7.42 (m, 3H), |
| | | | | | 6.65 (s, 1H), 5.58 (s, 1H), 5.37 (dtt, J = 56.8, 6.3, 3.5 Hz, 1H), 4.53 (ddd, J = |
| | | | | | 19.1, 11.4, 6.4 Hz, 2H), 4.38 (s, 2H). |
| 130 | C | A | 3.583 | 466.2 | 1H NMR (600 MHz, cdcl3) 8 9.25 (s, 1H), 8.53 (d, J = 37.7 Hz, 1H), 8.00-7.94 (m, 1H), |
| | | | | | 7.90 (d, J = 9.8 Hz, 1H), 7.56-7.42 (m, 3H), 6.63 (s, 1H), 5.68 (s, 1H), 5.28 (t, J = |
| | | | | | 55.2 Hz, 1H), 3.90 (d, J = 36.2 Hz, 4H), 3.75-3.52 (m, 2H). |
| 131 | B | C | 3.660 | 429.2 | ¹H NMR (400 MHz, Methanol-d₄) δ 7.86-7.73 (m, 2H), 7.72-7.56 (m, 4H), 7.51-7.39 (m, |
| | | | | | 2H), 2.34 (s, 3H). |
| 132 | B | A | 4.108 | 518.2 | |
| 133 | A | C | 2.900 | 489.2 | 1H NMR (600 MHz, Chloroform-d) δ 9.24 (s, 1H), 8.52 (d, J = 11.4 Hz, 1H), 7.96 (d, |
| | | | | | J = 8.8 Hz, 1H), 7.87 (dd, J = 8.7, 2.3 Hz, 1H), 7.55-7.50 (m, 2H), 7.47 (dd, J = |
| | | | | | 9.2, 7.1 Hz, 1H), 6.64 (s, 1H), 5.82 (s, 1H), 4.04-3.34 (m, 4H), 2.77 (d, J = 72.3 |
| | | | | | Hz, 1H), 2.33 (s, 3H), 2.24 (s, 3H), 2.11 (s, 1H), 1.89 (d, J = 11.9 Hz, 1H). |
| 134 | C | C | 3.470 | 431.2 | 1H NMR (600 MHz, Chloroform-d) δ 9.40 (s, 1H), 8.40 (d, J = 2.6 Hz, 1H), 8.17 (d, J = |
| | | | | | 9.0 Hz, 1H), 7.74 (dd, J = 9.0, 2.7 Hz, 1H), 7.57-7.52 (m, 2H), 7.49 (dd, J = 9.2, |
| | | | | | 7.0 Hz, 1H), 6.66 (s, 1H), 5.75 (s, 1H), 2.60 (s, 3H). |
| 135 | C | C | 3.420 | 450.2 | 1H NMR (400 MHz, Chloroform-d) δ 9.37 (s, 1H), 8.55 (s, 1H), 7.98 (s, 2H), 7.55-7.41 |
| | | | | | (m, 3H), 6.61 (s, 1H), 5.67 (s, 1H), 5.35 (d, J = 56.7 Hz, 1H), 4.50 (d, J = 15.9 Hz, |
| | | | | | 2H), 4.36 (s, 2H). |
| 136 | C | C | 3.429 | 464.2 | 1H NMR (600 MHz, cdcl3) δ 9.28 (s, 1H), 8.53 (d, J = 37.5 Hz, 1H), 7.98 (d, J = 8.8 |
| | | | | | Hz, 1H), 7.91 (d, J = 10.6 Hz, 1H), 7.57-7.43 (m, 3H), 6.63 (s, 1H), 5.73 (s, 1H), |
| | | | | | 5.19 (s, 1H), 3.97-3.65 (m, 4H), 2.30 (t, J = 15.8 Hz, 1H), 2.16-1.93 (m, 1H). |
| 137 | C | C | 3.500 | 446.2 | 1H NMR (600 MHz, cd3od) δ 8.47 (dd, J = 2.3, 0.9 Hz, 1H), 8.00-7.94 (m, 2H), 7.69- |
| | | | | | 7.60 (m, 3H), 3.58 (q, J = 7.1 Hz, 4H), 1.95 (dp, J = 36.3, 6.9 Hz, 4H). |
| 138 | | A | 3.680 | 479.2 | ¹H NMR (600 MHz, Chloroform-d) δ 8.62 (s, 1H), 7.58-7.54 (m, 2H), 7.50 (dd, J = 9.1, |
| | | | | | 4.8 Hz, 1H), 7.43-7.39 (m, 2H), 7.37 (dd, J = 9.1, 7.8 Hz, 1H), 6.62 (s, 1H), 5.55 |
| | | | | | (s, 1H), 3.78-3.57 (m, 8H). |
| 139 | C | B | 3.289 | 480.2 | 1H NMR (400 MHz, Methanol-d4) δ 8.97 (d, J = 2.7 Hz, 1H), 8.32 (dd, J = 8.6, 2.7 Hz, |
| | | | | | 1H), 7.83 (d, J = 8.6 Hz, 1H), 7.73-7.59 (m, 4H), 7.44 (dd, J = 9.3, 6.7 Hz, 1H), |
| | | | | | 6.83 (d, J = 6.9 Hz, 1H), 2.34 (s, 3H). |
| 140 | C | A | 3.450 | 462.2 | |
| 141 | C | C | 3.505 | 430.2 | ¹H NMR (400 MHz, Chloroform-d) δ 9.31 (s, 1H), 8.39 (dd, J = 2.6, 0.8 Hz, 1H), 8.11 |
| | | | | | (dd, J = 8.9, 0.8 Hz, 1H), 7.74 (dd, J = 8.9, 2.6 Hz, 1H), 7.57 (q, J = 0.8 Hz, 1H), |
| | | | | | 7.54-7.50 (m, 2H), 7.46 (dd, J = 9.5, 6.4 Hz, 1H), 6.62 (s, 1H), 5.63 (s, 1H), 2.32 |
| | | | | | (d, J = 0.8 Hz, 3H). |
| 142 | C | C | 3.497 | 444.2 | 1H NMR (400 MHz, Chloroform-d) d 9.28 (s, 1H), 8.60-8.52 (m, 1H), 8.06 (d, J = 8.7 |
| | | | | | Hz, 1H), 7.99-7.89 (m, 2H), 7.52 (dd, J = 7.9, 1.5 Hz, 2H), 7.46 (dd, J = 9.5, 6.4 |
| | | | | | Hz, 1H), 6.61 (s, 1H), 5.59 (s, 1H), 4.25 (q, J = 7.2 Hz, 2H), 1.50 (t, J = 7.2 Hz, |
| | | | | | 3H). |
| 143 | C | C | 3.630 | 434.2 | ¹H NMR (400 MHz, Chloroform-d) δ 8.84 (brs, 1H), 8.00 (d, J = 2.9 Hz, 1H), 7.85 (d, |
| | | | | | J = 9.0 Hz, 1H), 7.59-7.36 (m, 3H), 7.26 (s, 1H), 6.58 (brs, 1H), 5.55 (brs, 1H), |
| | | | | | 3.88-3.85 (m, 4H), 3.09-3.05 (m, 4H). |
| 144 | C | C | 3.920 | 497.2 | ¹H NMR (400 MHz, Chloroform-d) δ 9.25 (brs, 1H), 8.59-8.50 (m, 1H), 8.09-7.99 (m, 1H), |
| | | | | | 7.94 (dd, J = 8.8, 2.4 Hz, 1H), 7.57-7.43 (m, 3H), 7.32 (s, 1H), 6.62 (brs, 1H), 5.60 |
| | | | | | (brs, 1H), 3.76 (s, 3H). |
| 145 | A | A | 3.800 | 481.2 | ¹H NMR (600 MHz, Methanol-d₄) δ 7.71 (dd, J = 9.2, 4.9 Hz, 1H), 7.66-7.63 (m, 2H), |
| | | | | | 7.61 (d, J = 8.8 Hz, 1H), 7.56 (dd, J = 16.2, 8.4 Hz, 2H), 5.29 (dd, J = 61.6, 52.8 |
| | | | | | Hz, 1H), 3.90-3.66 (m, 4H), 2.33-2.17 (m, 1H), 2.17-2.02 (m, 1H). |
| 146 | A | A | 3.830 | 481.2 | ¹H NMR (600 MHz, Methanol-d₄) δ 7.65-7.61 (m, 2H), 7.59 (d, J = 8.1 Hz, 2H), 7.55 (dd, |
| | | | | | J = 16.1, 8.4 Hz, 2H), 5.29 (dd, J = 61.6, 52.7 Hz, 1H), 3.91-3.65 (m, 4H), 2.33-2.17 |
| | | | | | (m, 1H), 2.17-1.95 (m, 1H). |
| 147 | A | A | 3.720 | 465.2 | ¹H NMR (600 MHz, Methanol-d₄) δ 7.65-7.62 (m, 2H), 7.61-7.58 (m, 1H), 7.54 (dd, J = |
| | | | | | 16.1, 8.4 Hz, 2H), 7.46 (td, J = 9.1, 4.3 Hz, 1H), 5.38-5.15 (m, 1H), 3.91-3.63 (m, |
| | | | | | 4H), 2.32-2.17 (m, 1H), 2.16-2.02 (m, 1H). |
| 148 | A | A | 3.710 | 488.2 | ¹H NMR (600 MHz, Methanol-d₄) δ 8.15 (s, 2H), 7.64-7.60 (m, 2H), 7.54 (dd, J = 16.2, |
| | | | | | 8.4 Hz, 2H), 5.27 (dd, J = 61.9, 52.8 Hz, 1H), 3.89-3.63 (m, 4H), 2.31-2.16 (m, 1H), |
| | | | | | 2.15-1.99 (m, 1H). |
| 149 | A | C | 3.000 | 503.2 | 1H NMR (600 MHz, Chloroform-d) δ 9.22 (s, 1H), 8.39 (dd, J = 2.3, 0.8 Hz, 1H), 7.97 |
| | | | | | (dd, J = 8.7, 0.9 Hz, 1H), 7.75 (dd, J = 8.7, 2.3 Hz, 1H), 7.55-7.50 (m, 2H), 7.47 |
| | | | | | (dd, J = 9.1, 7.0 Hz, 1H), 6.65 (s, 1H), 6.03 (s, 1H), 2.94 (broad s, 2H), 2.80 (t, |
| | | | | | J = 11.8 Hz, 1H), 2.41 (s, 6H), 2.04 (s, 4H), 1.96 (s, 2H). |
| 150 | A | C | 2.940 | 489.2 | 1H NMR (400 MHz, Chloroform-d) δ 9.24 (s, 1H), 8.43 (s, 1H), 7.99 (dd, J = 8.7, 0.9 |
| | | | | | Hz, 1H), 7.82-7.70 (m, 1H), 7.57-7.46 (m, 3H), 6.67 (s, 1H), 5.94 (s, 1H), 3.98- |
| | | | | | 3.50(m, 4H), 2.92-2.45(m, 6H), 2.05 (d, J = 10.3 Hz, 3H). |
| 151 | D | C | 3190.000 | 476.2 | 1H NMR (600 MHz, Chloroform-d) δ 8.68 (s, 1H), 8.51 (s, 1H), 8.02 (dd, J = 8.7, 2.8 |
| | | | | | Hz, 1H), 7.52 (d, J = 7.4 Hz, 2H), 7.47 (dd, J = 9.2, 7.0 Hz, 1H), 7.34 (s, 1H), 6.64 |
| | | | | | (s, 1H), 5.64 (s, 1H), 3.93 (s, 2H), 3.62 (s, 6H), 3.56 (t, J = 4.6 Hz, 2H). |
| 152 | C | C | 3.240 | 464.2 | 1H NMR (600 MHz, Chloroform-d) δ 8.64 (d, J = 2.8 Hz, 1H), 8.44 (s, 1H), 8.00 (dd, |
| | | | | | J = 8.5, 2.8 Hz, 1H), 7.54-7.49 (m, 2H), 7.46 (dd, J = 9.1, 7.1 Hz, 1H), 7.30 (d, J = |
| | | | | | 8.5 Hz, 1H), 6.64(s, 1H), 5.63 (s, 1H), 5.27 (dtt, J = 56.6, 6.4, 3.5 Hz, 1H), 4.51 |
| | | | | | (dddd, J = 19.7, 10.6, 6.1, 1.7 Hz, 1H), 4.36-4.23 (m, 2H), 4.12 (dddd, J = 24.4, |
| | | | | | 11.6, 3.5, 1.7 Hz, 1H), 3.68-3.59 (m, 2H). |
| 153 | A | C | 3.300 | 457.2 | 1H NMR (600 MHz, Chloroform-d) δ 9.34 (s, 1H), 8.55 (dd, J = 2.3, 0.9 Hz, 1H), 7.99 |
| | | | | | (dd, J = 8.8, 0.9 Hz, 1H), 7.95 (dd, J = 8.8, 2.3 Hz, 1H), 7.56-7.50 (m, 2H), 7.48 |
| | | | | | (dd, J = 9.2, 6.9 Hz, 1H), 6.64 (S, 1H), 5.72 (s, 1H), 4.55 (t, J = 8.6 Hz, 2H), 4.50 |
| | | | | | (s, 2H), 3.58 (tt, J = 9.1, 6.3 Hz, 1H). |
| 154 | A | C | 3.030 | 448.2 | 1H NMR (600 MHz, Chloroform-d) δ 9.27 (s, 1H), 8.58 (dd, J = 2.2, 1.0 Hz, 1H), 8.02- |
| | | | | | 7.94 (m, 2H), 7.53 (d, J = 8.7 Hz, 2H), 7.48 (dd, J = 9.2, 7.0 Hz, 1H), 6.63 (s, 1H), |
| | | | | | 5.76 (s, 1H), 4.75 (ddt, J = 10.6, 8.2, 4.1 Hz, 1H), 4.51 (s, 2H), 4.22 (s, 1H), 4.08 |
| | | | | | (s, 1H). |
| 155 | C | C | 4.170 | 483.2 | ¹H NMR (600 MHz, Chloroform-d) δ 8.77 (brs, 1H), 8.09 (s, 1H), 7.76-7.68 (m, 2H), |
| | | | | | 7.56-7.50 (m, 2H), 7.50-7.39 (m, 3H), 6.65 (brs, 1H), 5.60 (brs, 1H). |
| 156 | C | C | 3.340 | 430.2 | 1H NMR (600 MHz, Chloroform-d) d 9.30 (s, 1H), 8.40 (d, J = 2.6 Hz, 1H), 8.10 (d, J = |
| | | | | | 8.8 Hz, 1H), 7.95 (s, 1H), 7.73 (dd, J = 8.9, 2.7 Hz, 1H), 7.57-7.51 (m, 2H), 7.48 |
| | | | | | (dd, J = 9.1, 7.0 Hz, 1H), 6.64 (s, 1H), 5.61 (s, 1H), 2.51 (s, 3H). |
| 157 | C | C | 4.030 | 484.2 | ¹H NMR (600 MHz, Chloroform-d) δ 9.41 (s, 1H), 8.45 (d, J = 2.6 Hz, 1H), 8.18-8.11 (m, |
| | | | | | 2H), 7.76 (dd, J = 9.0, 2.7 Hz, 1H), 7.54 (d, J = 8.6 Hz, 2H), 7.49 (dd, J = 9.1, 7.0 |
| | | | | | Hz, 1H), 6.64 (s, 1H), 5.63 (s, 1H). |
| 158 | C | C | 3.410 | 431.2 | ¹H NMR (400 MHz, Chloroform-d) δ 9.39 (brs, 1H), 8.69 (dd, J = 2.3, 0.9 Hz, 1H), 8.20- |
| | | | | | 7.98 (m, 2H), 7.63-7.40 (m, 3H), 6.65 (s, 1H), 5.63 (brs, 1H), 4.20 (brs, 3H). |
| 159 | C | C | 4.130 | 484.2 | |
| 160 | C | C | 3.220 | 510.0 | 1H NMR (400 MHz, Chloroform-d) δ 9.38 (s, 1H), 8.47 (s, 1H), 8.04 (s, 1H), 7.81 (d, |
| | | | | | J = 7.5 Hz, 1H), 7.63-7.46 (m, 3H), 6.67 (s, 1H), 5.85 (s, 1H), 4.16 (s, 4H), 3.12 |
| | | | | | (s, 4H). |
| 161 | C | B | 3.229 | 481.2 | |
| 162 | A | A | 3.510 | 454.2 | 1H NMR (600 MHz, Methanol-d₄) δ 8.03 (dd, J = 8.3, 1.3 Hz, 1H), 7.98 (dd, J = 7.8, 1.3 |
| | | | | | Hz, 1H), 7.78 (t, J = 8.1 Hz, 1H), 7.69-7.66 (m, 2H), 7.57 (dd, J = 16.1, 8.4 Hz, |
| | | | | | 2H), 5.29 (dd, J = 61.6, 52.8 Hz, 1H), 3.90-3.67 (m, 4H), 2.33-2.17 (m, 1H), 2.17- |
| | | | | | 2.01 (m, 1H). |
| 163 | A | C | 3.350 | 509.0 | 1H NMR (400 MHz, Chloroform-d) δ 8.67 (s, 1H), 7.62-7.55 (m, 2H), 7.54-7.48 (m, 2H), |
| | | | | | 7.48-7.43 (m, 1H), 7.43-7.38 (m, 2H), 6.63 (s, 1H), 5.67 (s, 1H), 4.08 (d, J = 5.4 |
| | | | | | Hz, 4H), 3.04 (s, 4H). |
| 164 | A | A | 3.440 | 438.2 | |
| 165 | A | C | 3.750 | 460.2 | 1H NMR (400 MHz, Chloroform-d) δ 9.32 (s, 1H), 8.58 (d, J = 2.1 Hz, 1H), 8.02 (dd, |
| | | | | | J = 8.8, 2.0 Hz, 1H), 7.97 (d, J = 8.7 Hz, 1H), 7.58-7.49 (m, 2H), 7.51-7.43 (m, 1H), |
| | | | | | 6.63 (s, 1H), 5.73 (s, 1H), 4.01 (s, 2H), 3.89 (s, 2H), 1.31 (s, 6H). |
| 166 | A | C | 4.060 | 459.0 | 1H NMR (400 MHz, Chloroform-d) δ 8.63 (s, 1H), 7.68-7.60 (m, 2H), 7.58-7.43 (m, 5H), |
| | | | | | 6.65 (s, 1H), 5.75 (s, 1H), 3.98 (s, 2H), 3.88 (s, 2H), 1.29 (s, 6H). |
| 167 | A | A | 3.730 | 465.2 | |
| 168 | C | C | 3.020 | 465.2 | 1H NMR (600 MHz, cdcl3) δ 7.87-7.82 (m, 2H), 7.54 (d, J = 0.8 Hz, 1H), 7.50 (d, J = |
| | | | | | 1.3 Hz, 1H), 7.49 (d, J = 0.6 Hz, 1H), 7.44 (dd, J = 9.1, 7.2 Hz, 1H), 6.56 (s, 1H), |
| | | | | | 5.54(s, 1H), 4.93 (s, 2H), 3.65-3.50 (m, 8H). |
| 169 | D | C | 3.060 | 508.0 | 1H NMR (400 MHz, Methanol-d4) δ 7.69-7.55 (m, 5H), 7.52-7.44 (m, 2H), 4.42 (s, 2H), |
| | | | | | 3.87-3.76 (m, 2H), 3.74-3.64 (m, 4H). |
| 170 | A | C | 3.800 | 477.2 | 1H NMR (400 MHz, Methanol-d4) δ 7.67-7.54 (m, 5H), 7.40-7.30 (m, 2H), 3.84 (s, 4H), |
| | | | | | 2.64 (S, 4H). |
| 171 | A | C | 3.190 | 522.2 | 1H NMR (400 MHz, Methanol-d4) δ 7.68-7.57 (m, 5H), 7.50-7.44 (m, 2H), 4.21 (s, 2H), |
| | | | | | 3.83 (ddd, J = 13.1, 8.6, 3.1 Hz, 2H), 3.52-3.32 (m, 4H), 2.80 (s, 3H). |
| 172 | C | B | 3.051 | 416.0 | |
| 173 | C | C | 2.472 | 430.0 | |
| 174 | C | C | 2.986 | 513.0 | 1H NMR (400 MHz, CDCl3) δ 7.97-7.84 (m, 1H), 7.72-7.32 (m, 4H), 6.60 (s, 1H), 5.64 |
| | | | | | (s, 1H), 5.01 (s, 2H), 4.11 (t, J = 5.5 Hz, 3H), 2.92 (d, J = 62.5 Hz, 3H). |
| 175 | A | C | 3.440 | 527.1 | 1H NMR (400 MHz, Chloroform-d) δ 8.72 (s, 1H), 7.66-7.58 (m, 2H), 7.49-7.42 (m, 2H), |
| | | | | | 7.32 (d, J = 7.7 Hz, 2H), 6.65 (s, 1H), 5.61 (s, 1H), 4.14 (s, 4H), 3.09 (d, J = 6.5 |
| | | | | | Hz, 4H). |
| 176 | A | C | 3.920 | 507.0 | 1H NMR (400 MHz, Chloroform-d) δ 8.61 (s, 1H), 7.63-7.55 (m, 2H), 7.43-7.35 (m, 2H), |
| | | | | | 7.04 (s, 2H), 6.65 (s, 1H), 5.62 (s, 1H), 3.91 (s, 4H), 2.67 (s, 4H). |
| 177 | A | C | 3.440 | 527.1 | 1H NMR (400 MHz, Chloroform-d) δ 8.72 (s, 1H), 7.66-7.58 (m, 2H), 7.49-7.42 (m, 2H), |
| | | | | | 7.32 (d, J = 7.7 Hz, 2H), 6.65 (s, 1H), 5.61 (s, 1H), 4.14 (s, 4H), 3.09 (d, J = 6.5 |
| | | | | | Hz, 4H). |

### Example 23

The compounds listed in Table 1a were tested for their ability to inhibit a set of kinases according to the following procedures.

Biochemical Assays:

The basic protocol for TR-FRET LanthaScreen Eu Kinase Binding Assay inhibitor studies were performed as follows. LanthaScreen Kinase Binding Assays (ThermoFisher/USA) to evaluate inhibitors were performed by addition of 5 μL of test compound in corresponding DMSO dilutions/5 μl of kinase/antibody mixture, 5 μL of tracer into 384 well small volume plates. After incubation for 1 hour at room temperature, plates were read. Data analysis of emission ratios was according LanthaScreen Eu Kinase Binding Assay protocol.

Kinase and assay components were adjusted to final concentrations according to kit protocol. For LRRK2: 5 nM wt human LRRK2, catalytic site, or G2019S human LRRK2, catalytic site (ThermoFisher/USA), 2 nM Eu-Anti-GST Antibody, 10 nM Kinase Tracer 236 in 1× Kinase Buffer A. For NUAK1: 8 nM wt human NUAK1, full length (ThermoFisher/USA), 2 nM Eu-Anti-His Antibody, 5 nM Kinase Tracer 236 in 1× Kinase Buffer A.

Basic protocol for Z′-LYTE Assays (ThermoFisher/USA) inhibitor studies involved two steps:
Kinase reaction: Addition of 2 μL of test compound in corresponding DMSO dilutions, 5 L of kinase/substrate mixture, 3 μL of ATP into 384 well small volume plates. Incubation for at least 1 hour at room temperature.
Development reaction: Addition of 5 μL development reagent, incubation for 1 hour at room temperature, addition of 5 μL stop reagent, read plate. Data analysis of emission ratios according to Z′-LYTE assay protocol.

Kinase and assay components were adjusted to final concentrations according to kit protocol. For JAK2: 5 nM wt human JAK2, catalytic site (ThermoFisher/USA), 2 μM Z′-LYTE Tyr 06 Peptide, 36 μM ATP in Kinase Buffer.

Basic protocol for HTRF KinEASE assay (Cisbio/FRA) inhibitor studies involved two steps:
Enzymatic step: Addition of 4 μL of test compound in corresponding DMSO dilutions, 4 μL of kinase/substrate mixture, 2 μL of ATP into 384 well small volume plates. Incubation for at least 30 minutes at room temperature.
Detection step: Addition of 5 μL antibody and 5 μL streptavidin-XL665, read plate after 60 minutes. Data analysis of emission ratios according KinEASE assay protocol.

Kinase and assay components were adjusted to final concentrations according to kit protocol. For TYK2: 2 nM wt human TYK2, catalytic site (SignalChem/CAN), 1 μM HTRF KinEASE-TK Substrate-biotin, 1 μM ATP in 1× Kinase Buffer. For JAK1: 4 nM wt human JAK1, catalytic site (ThermoFisher/USA), 1 μM HTRF KinEASE-TK Substrate-biotin, 2.5 μM ATP in 1× Kinase Buffer. For JAK2: 1 nM wt human JAK2, catalytic site (SignalChem/CAN), 1 μM HTRF KinEASE-TK Substrate-biotin, 100 μM ATP in 1× Kinase Buffer. Results for from biochemical tests were as follows:

### Activity Toward TYK2

The following compounds had an IC₅₀ lower than 10 nM: Compound Nos. 101, 102, 104, 105, 106, 107, 108, 109, 112, 114, 116, 122, 124, 128, 129, 131, 160, 162, 163, 169, 170, 171, and 175.

The following compounds had an IC₅₀ between 10 nM and 100 nM: Compound Nos. 103, 110, 113, 115, 117, 119, 120, 123, 125, 126, 130, 134, 135, 137, 138, 140, 141, 144, 145, 146, 147, 148, 149, 150, 153, 154, 156, 158, 161, 164, 165, 166, 167, 168, 174, 177 and 477.

The following compounds had an IC₅₀ between 100 nM and 1000 nM: Compound Nos. 111, 118, 127, 132, 133, 139, 142, 143, 151, 152, 157, and 176.

Activity Toward LRRK2(wt) and LRRK2(G2019) The following compounds had an IC₅₀ between 100 nM and 1000 nM: Compound Nos. 108, 113, 116, 122, 129, 129, and 131.

### Activity Toward NUAK1

The following compounds had an IC₅₀ between 100 nM and 1000 nM: Compound Nos. 101, 102, 103, 104, 105, and 106.

### Activity Toward JAK1

The following compounds had an IC₅₀ lower than 10 nM: Compound Nos. 101, 102, 105, 108, 112, 113, 114, 123, 126, 128, 129, 138, 145, 146, 147, 149, 163, 166, 169, 170, 171, 174, 175, and 177.

The following compounds had an IC₅₀ between 10 nM and 100 nM: Compound Nos. 103, 104, 106, 109, 110, 116, 117, 118, 119, 120, 122, 124, 125, 131, 133, 134, 135, 137, 139, 141, 142, 144, 150, 151, 152, 153, 154, 155, 156, 157, 158, 160, 161, 162, 164, 167, 168, and 176.

The following compounds had an IC₅₀ between 100 nM and 1000 nM: Compound Nos. 107, 111, 115, 121, 127, 130, 132, 136, 140, 143, 148, 165, and 172.

### Activity Toward JAK2

The following compounds had an IC₅₀ between 100 nM and 1000 nM: Compound Nos. 101, 102, 103, 104, 105, 106, 107, 108, 109, 113, 114, 116, 117, 119, 120, 122, 124, 125, 129, 131, 138, 145, 147, 162, 163, 164, 167, 169, and 171.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A compound of Formula (II): 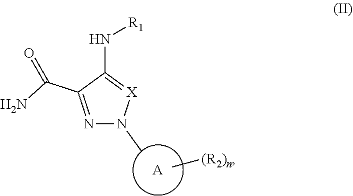
wherein:
X is N or CH;
R₁ is C(O)CH₂R₃, optionally substituted cycloalkyl, optionally substituted cycloheteroalkyl, optionally substituted cycloalkenyl, optionally substituted cycloheteroalkenyl, optionally substituted aryl, or optionally substituted heteroaryl;
each instance of R₂ is independently halo, haloalkyl, cyano, optionally substituted alkoxyl, optionally substituted alkyl, optionally substituted heteroalkyl, optionally substituted alkenyl, optionally substituted heteroalkenyl;
R₃ is optionally substituted cycloalkyl, optionally substituted cycloheteroalkyl, optionally substituted cycloalkenyl, optionally substituted cycloheteroalkenyl, optionally substituted aryl, or optionally substituted heteroaryl;
A is cycloalkyl, cycloheteroalkyl, cycloalkenyl, cycloheteroalkenyl, aryl, or heteroaryl; and
n is 0-5.

2. The compound of claim 1, wherein X is N.

3. The compound of claim 1, wherein X is CH.

4. The compound of claim 1, wherein A is aryl or heteroaryl.

5. The compound of claim 4, wherein each instance of R₂ is halo or cyano.

6. The compound of claim 5, wherein n is 2 or 3.

7. The compound of claim 6, wherein the compound is represented by Formula (IIa):

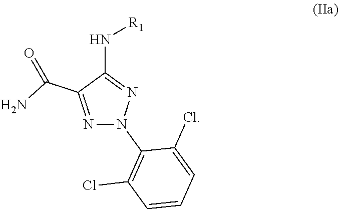

8. The compound of claim 6, wherein the compound is represented by Formula (I):

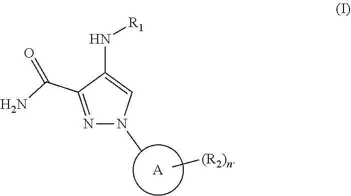

9. The compound of claim 1, wherein R₁ is an optionally substituted aryl.

10. The compound of claim 9, wherein R₁ is a substituted phenyl.

11. The compound of claim 1, wherein R₁ is optionally substituted aryl or optionally substituted heteroaryl;

12. The compound of claim 11, wherein R₁ is an optionally substituted aryl other than phenyl.

13. The compound of claim 11, wherein R₁ is an optionally substituted heteroaryl.

14. The compound of claim 11, wherein A is aryl or heteroaryl.

15. The compound of claim 14, wherein A is aryl.

16. The compound of claim 14, wherein A is heteroaryl.

17. The compound of claim 14, wherein each instance of R₂ is halo or cyano.

18. The compound of claim 17, wherein n is 2 or 3.

19. The compound of claim 1, wherein the compound is selected from the group consisting of 1-(4-methoxyphenyl)-4-(2-morpholinoacetamido)-1H-pyrazole-3-carboxamide; 1-(2,6-difluorophenyl)-4-(2-morpholinoacetamido)-1H-pyrazole-3-carboxamide; 1-(2-fluorophenyl)-4-(2-morpholinoacetamido)-1H-pyrazole-3-carboxamide; 1-(2,6-difluorophenyl)-4-((6-methylpyrimidin-4-yl)amino)-1H-pyrazole-3-carboxamide; 1-(2-fluorophenyl)-4-((6-methylpyrimidin-4-yl)amino)-1H-pyrazole-3-carboxamide; 4-((5-aminopyridin-2-yl)amino)-1-(2,6-difluorophenyl)-1H-pyrazole-3-carboxamide; 1-(2,6-difluorophenyl)-4-(pyridin-2-ylamino)-1H-pyrazole-3-carboxamide; 4-((2-amino-6-methylpyrimidin-4-yl)amino)-1-(2,6-difluorophenyl)-1H-pyrazole-3-carboxamide; 4-(benzo[d]thiazol-2-ylamino)-1-(2,6-difluorophenyl)-1H-pyrazole-3-carboxamide; 1-(2,6-difluorophenyl)-4-((4-methylpyridin-2-yl)amino)-1H-pyrazole-3-carboxamide; 4-((1H-benzo[d]imidazol-2-yl)amino)-1-(2,6-difluorophenyl)-1H-pyrazole-3-carboxamide; 1-(2,6-difluorophenyl)-4-((4-(morpholine-4-carbonyl)phenyl)amino)-1H-pyrazole-3-carboxamide; 1-(2,6-difluorophenyl)-4-((4-((tetrahydro-2H-pyran-4-yl)carbamoyl)phenyl)amino)-1H-pyrazole-3-carboxamide; 1-(2,6-difluorophenyl)-4-((4-((2,2,2-trifluoroethyl)carbamoyl)phenyl)amino)-1H-pyrazole-3-carboxamide; 4-((4-(tert-butylcarbamoyl)phenyl)amino)-1-(2,6-difluorophenyl)-1H-pyrazole-3-carboxamide; 1-(2,6-difluorophenyl)-4-((4-(ethylcarbamoyl)phenyl)amino)-1H-pyrazole-3-carboxamide; 1-(2,6-dichlorophenyl)-4-((4-(morpholine-4-carbonyl)phenyl)amino)-1H-pyrazole-3-carboxamide; 1-(2,6-difluorophenyl)-4-((3-(morpholine-4-carbonyl)phenyl)amino)-1H-pyrazole-3-carboxamide; 4-((4-chlorophenyl)amino)-1-(2,6-difluorophenyl)-1H-pyrazole-3-carboxamide; 4-((4-chlorophenyl)amino)-1-(2,6-dichlorophenyl)-1H-pyrazole-3-carboxamide; 1-(2,6-dichlorophenyl)-4-(phenylamino)-1H-pyrazole-3-carboxamide; 1-(2-chloro-6-fluorophenyl)-4-((4-(morpholine-4-carbonyl)phenyl)amino)-1H-pyrazole-3-carboxamide; N-(tert-butyl)-5-((3-carbamoyl-1-(2,6-difluorophenyl)-1H-pyrazol-4-yl)amino)picolinamide; 5-((3-carbamoyl-1-(2,6-difluorophenyl)-1H-pyrazol-4-yl)amino)-N-ethylpicolinamide; 5-((3-carbamoyl-1-(2-fluoro-6-methoxyphenyl)-1H-pyrazol-4-yl)amino)-N-ethylpicolinamide; 4-((4-(tert-butylcarbamoyl)phenyl)amino)-1-(2,6-dichlorophenyl)-1H-pyrazole-3-carboxamide; 4-((4-(tert-butylcarbamoyl)phenyl)amino)-1-(2-chloro-6-fluorophenyl)-1H-pyrazole-3-carboxamide; 4-((4-(tert-butylcarbamoyl)-3-fluorophenyl)amino)-1-(2,6-dichlorophenyl)-1H-pyrazole-3-carboxamide; 4-((4-(tert-butylcarbamoyl)-3-fluorophenyl)amino)-1-(2-chloro-6-fluorophenyl)-1H-pyrazole-3-carboxamide; 1-(2,6-difluorophenyl)-4-((6-(morpholine-4-carbonyl)pyridin-3-yl)amino)-1H-pyrazole-3-carboxamide; 1-(2,6-dichlorophenyl)-4-((4-((1-methylpiperidin-4-yl)carbamoyl)phenyl)amino)-1H-pyrazole-3-carboxamide; 1-(2,6-dichlorophenyl)-4-((4-(4-methylpiperazine-1-carbonyl)phenyl)amino)-1H-pyrazole-3-carboxamide; tert-butyl_3-(4-((3-carbamoyl-1-(2,6-dichlorophenyl)-1H-pyrazol-4-yl)amino)benzamido)piperidine-1-carboxylate; 1-(2,6-dichlorophenyl)-4-((4-(ethylcarbamoyl)phenyl)amino)-1H-pyrazole-3-carboxamide; 1-(2-chloro-6-fluorophenyl)-4-((4-((1-methylpiperidin-4-yl)carbamoyl)phenyl)amino)-1H-pyrazole-3-carboxamide; 1-(2-chloro-6-fluorophenyl)-4-((4-(4-methylpiperazine-1-carbonyl)phenyl)amino)-1H-pyrazole-3-carboxamide; tert-butyl_3-(4-((3-carbamoyl-1-(2-chloro-6-fluorophenyl)-1H-pyrazol-4-yl)amino)benzamido)piperidine-1-carboxylate; 1-(2-chloro-6-fluorophenyl)-4-((4-(piperidin-3-ylcarbamoyl)phenyl)amino)-1H-pyrazole-3-carboxamide; 1-(2,6-dichlorophenyl)-4-((4-(piperidin-3-ylcarbamoyl)phenyl)amino)-1H-pyrazole-3-carboxamide; 1-(2-chloro-6-fluorophenyl)-4-((4-(ethylcarbamoyl)phenyl)amino)-1H-pyrazole-3-carboxamide; 1-(2,6-dichlorophenyl)-4-((4-pivaloylphenyl)amino)-1H-pyrazole-3-carboxamide; 1-(2-chloro-6-fluorophenyl)-4-((4-pivaloylphenyl)amino)-1H-pyrazole-3-carboxamide; 1-(2,6-dichlorophenyl)-4-((1,2-dimethyl-1H-benzo[d]imidazol-5-yl)amino)-1H-pyrazole-3-carboxamide; 1-(2-chloro-6-fluorophenyl)-4-((1,2-dimethyl-1H-benzo[d]imidazol-5-yl)amino)-1H-pyrazole-3-carboxamide; 1-(2,6-dichlorophenyl)-4-((4-(4-methylpyridazin-3-yl)phenyl)amino)-1H-pyrazole-3-carboxamide; 4-((4-(2-azaspiro[3.3]heptane-2-carbonyl)phenyl)amino)-1-(2,6-dichlorophenyl)-1H-pyrazole-3-carboxamide; 1-(2,6-dichlorophenyl)-4-((4-(1-ethyl-1H-1,2,4-triazol-5-yl)phenyl)amino)-1H-pyrazole-3-carboxamide; 1-(2,6-dichlorophenyl)-4-((4-(1-methyl-1H-1,2,3-triazol-5-yl)phenyl)amino)-1H-pyrazole-3-carboxamide; 1-(2,6-dichlorophenyl)-4-((4-(pyridazin-3-yl)phenyl)amino)-1H-pyrazole-3-carboxamide; 1-(2,6-dichlorophenyl)-4-((4-(methyl(1-methylpiperidin-4-yl)carbamoyl)phenyl)amino)-1H-pyrazole-3-carboxamide; 1-(2,6-dichlorophenyl)-4-((4-(5-methylpyrimidin-4-yl)phenyl)amino)-1H-pyrazole-3-carboxamide; 1-(2,6-dichlorophenyl)-4-((4-(5-ethylpyrimidin-4-yl)phenyl)amino)-1H-pyrazole-3-carboxamide; 1-(2,6-dichlorophenyl)-4-((4-(1,4-dimethyl-1H-imidazol-5-yl)phenyl)amino)-1H-pyrazole-3-carboxamide; 4-((4-(2-azaspiro[3.3]heptane-2-carbonyl)phenyl)amino)-1-(2-chloro-6-fluorophenyl)-1H-pyrazole-3-carboxamide; 1-(2,6-dichlorophenyl)-4-((4-(5,6,7,8-tetrahydro-[1,2,4]triazolo[4,3-a]pyridin-3-yl)phenyl)amino)-1H-pyrazole-3-carboxamide; 1-(2-chloro-6-fluorophenyl)-4-((4-(4-methylpyridazin-3-yl)phenyl)amino)-1H-pyrazole-3-carboxamide; 1-(2,6-dichlorophenyl)-4-((4-(6,8-dihydro-5H-[1,2,4]triazolo[3,4-c][1,4]oxazin-3-yl)phenyl)amino)-1H-pyrazole-3-carboxamide; 1-(2-chloro-6-fluorophenyl)-4-((4-(5,6,7,8-tetrahydro-[1,2,4]triazolo[4,3-a]pyridin-3-yl)phenyl)amino)-1H-pyrazole-3-carboxamide; 4-((4-(3-cyanopyridin-2-yl)phenyl)amino)-1-(2,6-dichlorophenyl)-1H-pyrazole-3-carboxamide; 4-((4-(4-cyanopyridazin-3-yl)phenyl)amino)-1-(2,6-dichlorophenyl)-1H-pyrazole-3-carboxamide; 4-((4-(2-azaspiro[3.3]heptane-2-carbonyl)phenyl)amino)-1-(2-fluoro-6-(trifluoromethyl)phenyl)-1H-pyrazole-3-carboxamide; 1-(2-chloro-6-fluorophenyl)-4-((4-(6,8-dihydro-5H-[1,2,4]triazolo[3,4-c][1,4]oxazin-3-yl)phenyl)amino)-1H-pyrazole-3-carboxamide; 1-(2,6-dichlorophenyl)-4-((4-(6,7,8,9-tetrahydro-5H-[1,2,4]triazolo[4,3-a]azepin-3-yl)phenyl)amino)-1H-pyrazole-3-carboxamide; 1-(2,6-difluorophenyl)-4-((4-(dimethylphosphoryl)phenyl)amino)-1H-pyrazole-3-carboxamide; 4-((4-(1H-indazol-1-yl)phenyl)amino)-1-(2,6-dichlorophenyl)-1H-pyrazole-3-carboxamide; 4-((4-(2H-indazol-2-yl)phenyl)amino)-1-(2,6-dichlorophenyl)-1H-pyrazole-3-carboxamide; 4-((4-(4-chloropyridazin-3-yl)phenyl)amino)-1-(2,6-dichlorophenyl)-1H-pyrazole-3-carboxamide; 1-(2-chloro-6-fluorophenyl)-4-((5-(morpholine-4-carbonyl)pyridin-2-yl)amino)-1H-pyrazole-3-carboxamide; 1-(2,6-dichlorophenyl)-4-((4-(4-(trifluoromethyl)pyridazin-3-yl)phenyl)amino)-1H-pyrazole-3-carboxamide; 1-(2,6-dichlorophenyl)-4-((4-(6,7-dihydropyrano[4,3-c]pyrazol-1(4H)-yl)phenyl)amino)-1H-pyrazole-3-carboxamide; 1-(2,6-dichlorophenyl)-4-((4-(6,7-dihydropyrano[4,3-c]pyrazol-2(4H)-yl)phenyl)amino)-1H-pyrazole-3-carboxamide; 1-(2,6-dichlorophenyl)-4-((4-(3-methyl-6-oxopyridazin-1(6H)-yl)phenyl)amino)-1H-pyrazole-3-carboxamide; 1-(2,6-dichlorophenyl)-4-((4-(4,5,6,7-tetrahydro-1H-benzo[d][1,2,3]triazol-1-yl)phenyl)amino)-1H-pyrazole-3-carboxamide; 1-(2,6-dichlorophenyl)-4-((5-(morpholine-4-carbonyl)pyridin-2-yl)amino)-1H-pyrazole-3-carboxamide; 1-(2,6-dichlorophenyl)-4-((4-(3-methyl-1H-pyrazol-1-yl)phenyl)amino)-1H-pyrazole-3-carboxamide; 4-((4-(3-chloro-1H-pyrazol-1-yl)phenyl)amino)-1-(2,6-dichlorophenyl)-1H-pyrazole-3-carboxamide; 1-(2,6-dichlorophenyl)-4-((4-(2-methyl-5-oxo-2,5-dihydro-1H-pyrazol-1-yl)phenyl)amino)-1H-pyrazole-3-carboxamide; 1-(2,6-dichlorophenyl)-4-((4-((1-methyl-1H-pyrazol-3-yl)oxy)phenyl)amino)-1H-pyrazole-3-carboxamide; 4-((4-(3-chloro-1H-1,2,4-triazol-1-yl)phenyl)amino)-1-(2,6-dichlorophenyl)-1H-pyrazole-3-carboxamide; 1-(2,6-dichlorophenyl)-4-((4-(5-methyl-1H-pyrazol-1-yl)phenyl)amino)-1H-pyrazole-3-carboxamide; 4-((4-(1H-benzo[d][1,2,3]triazol-1-yl)phenyl)amino)-1-(2,6-dichlorophenyl)-1H-pyrazole-3-carboxamide; 1-(2,6-dichlorophenyl)-4-((4-(piperidine-1-carbonyl)phenyl)amino)-1H-pyrazole-3-carboxamide; 1-(2,6-dichlorophenyl)-4-((4-(4-(2,2-difluoroethyl)piperazine-1-carbonyl)phenyl)amino)-1H-pyrazole-3-carboxamide; 4-((4-(4-cyclopropylpiperazine-1-carbonyl)phenyl)amino)-1-(2,6-dichlorophenyl)-1H-pyrazole-3-carboxamide; 1-(2,6-dichlorophenyl)-4-((4-((4-methylpiperazin-1-yl)sulfonyl)phenyl)amino)-1H-pyrazole-3-carboxamide; 4-((4-(4-(cyclopropylmethyl)piperazine-1-carbonyl)phenyl)amino)-1-(2,6-dichlorophenyl)-1H-pyrazole-3-carboxamide; 1-(2,6-dichlorophenyl)-4-((4-(4-ethyl-4H-1,2,4-triazol-3-yl)phenyl)amino)-1H-pyrazole-3-carboxamide; 1-(2,6-dichlorophenyl)-4-((4-(4-methyl-4H-1,2,4-triazol-3-yl)phenyl)amino)-1H-pyrazole-3-carboxamide; 1-(2,6-dichlorophenyl)-4-((4-(N-(1-methylpiperidin-4-yl)sulfamoyl)phenyl)amino)-1H-pyrazole-3-carboxamide; 1-(2,6-dichlorophenyl)-4-((4-(1-isopropyl-1H-1,2,4-triazol-5-yl)phenyl)amino)-1H-pyrazole-3-carboxamide; 1-(2,6-dichlorophenyl)-4-((4-(4-methyl-1,4-diazepane-1-carbonyl)phenyl)amino)-1H-pyrazole-3-carboxamide; 4-((4-(azetidine-1-carbonyl)phenyl)amino)-1-(2,6-dichlorophenyl)-1H-pyrazole-3-carboxamide; 1-(2,6-dichlorophenyl)-4-((4-(4-(2,2,2-trifluoroethyl)piperazine-1-carbonyl)phenyl)amino)-1H-pyrazole-3-carboxamide; 1-(2,6-dichlorophenyl)-4-((4-(pyrrolidine-1-carbonyl)phenyl)amino)-1H-pyrazole-3-carboxamide; 4-((4-(azepane-1-carbonyl)phenyl)amino)-1-(2,6-dichlorophenyl)-1H-pyrazole-3-carboxamide; 1-(2,6-dichlorophenyl)-4-((4-(1-methyl-1H-1,2,4-triazol-5-yl)phenyl)amino)-1H-pyrazole-3-carboxamide; 1-(2,6-dichlorophenyl)-4-((4-(2-oxobenzo[d]thiazol-3(2H)-yl)phenyl)amino)-1H-pyrazole-3-carboxamide; 4-((4-(4-cyclopentyl-4H-1,2,4-triazol-3-yl)phenyl)amino)-1-(2,6-dichlorophenyl)-1H-pyrazole-3-carboxamide; 1-(2,6-dichlorophenyl)-4-((4-(4-isobutyl-4H-1,2,4-triazol-3-yl)phenyl)amino)-1H-pyrazole-3-carboxamide; 1-(2,6-dichlorophenyl)-4-((4-(1-methyl-1H-imidazol-5-yl)phenyl)amino)-1H-pyrazole-3-carboxamide; 1-(2,6-dichlorophenyl)-4-((4-(4-isopropyl-4H-1,2,4-triazol-3-yl)phenyl)amino)-1H-pyrazole-3-carboxamide; 4-((4-(4-cyclopropyl-4H-1,2,4-triazol-3-yl)phenyl)amino)-1-(2,6-dichlorophenyl)-1H-pyrazole-3-carboxamide; 1-(2-chloro-6-fluorophenyl)-4-((4-(4-ethyl-4H-1,2,4-triazol-3-yl)phenyl)amino)-1H-pyrazole-3-carboxamide; 1-(2-chloro-6-fluorophenyl)-4-((4-(6,7-dihydropyrano[4,3-c]pyrazol-1(4H)-yl)phenyl)amino)-1H-pyrazole-3-carboxamide; 1-(2-chloro-6-fluorophenyl)-4-((4-(6,7-dihydropyrano[4,3-c]pyrazol-2(4H)-yl)phenyl)amino)-1H-pyrazole-3-carboxamide; 1-(2,6-dichlorophenyl)-4-((4-(4-(2,2,2-trifluoroethyl)-4H-1,2,4-triazol-3-yl)phenyl)amino)-1H-pyrazole-3-carboxamide; 1-(2-chloro-6-fluorophenyl)-4-((4-(1-isopropyl-1H-1,2,4-triazol-5-yl)phenyl)amino)-1H-pyrazole-3-carboxamide; 1-(2,6-dichlorophenyl)-4-((4-(4-(2,2-difluoroethyl)-4H-1,2,4-triazol-3-yl)phenyl)amino)-1H-pyrazole-3-carboxamide; 1-(2,6-dichlorophenyl)-4-((4-(4,5-dimethylpyridazin-3-yl)phenyl)amino)-1H-pyrazole-3-carboxamide; 1-(2,6-dichlorophenyl)-4-((4-(4-(2-(dimethylamino)ethyl)-4H-1,2,4-triazol-3-yl)phenyl)amino)-1H-pyrazole-3-carboxamide; 1-(2,6-dichlorophenyl)-4-((4-(1-methyl-3-(trifluoromethyl)-1H-1,2,4-triazol-5-yl)phenyl)amino)-1H-pyrazole-3-carboxamide; 1-(2,6-dichlorophenyl)-4-((4-(3,5-dimethyl-1H-pyrazol-1-yl)phenyl)amino)-1H-pyrazole-3-carboxamide; 1-(2,6-dichlorophenyl)-4-((4-(3,4-dimethyl-1H-pyrazol-1-yl)phenyl)amino)-1H-pyrazole-3-carboxamide; 1-(2,6-dichlorophenyl)-4-((4-(4,5-dimethyl-1H-pyrazol-1-yl)phenyl)amino)-1H-pyrazole-3-carboxamide; 4-((4-(4-chloro-3,5-dimethyl-1H-pyrazol-1-yl)phenyl)amino)-1-(2,6-dichlorophenyl)-1H-pyrazole-3-carboxamide; 4-((4-(4-chloro-5-methyl-1H-pyrazol-1-yl)phenyl)amino)-1-(2,6-dichlorophenyl)-1H-pyrazole-3-carboxamide; 4-((4-(4-chloro-3-methyl-1H-pyrazol-1-yl)phenyl)amino)-1-(2,6-dichlorophenyl)-1H-pyrazole-3-carboxamide; 1-(2,6-dichlorophenyl)-4-((4-(4-ethyl-3,5-dimethyl-1H-pyrazol-1-yl)phenyl)amino)-1H-pyrazole-3-carboxamide; 1-(2,6-dichlorophenyl)-4-((4-(3,5-diethyl-1H-pyrazol-1-yl)phenyl)amino)-1H-pyrazole-3-carboxamide; 1-(2,6-dichlorophenyl)-4-((4-(3,4,5-trimethyl-1H-pyrazol-1-yl)phenyl)amino)-1H-pyrazole-3-carboxamide; 1-(2,6-dichlorophenyl)-4-((4-(1-methyl-1H-imidazol-2-yl)phenyl)amino)-1H-pyrazole-3-carboxamide; 1-(2,6-dichlorophenyl)-4-((5-(pyrrolidine-1-carbonyl)pyridin-2-yl)amino)-1H-pyrazole-3-carboxamide; 1-(2-chloro-6-fluorophenyl)-4-((4-(pyrrolidine-1-carbonyl)phenyl)amino)-1H-pyrazole-3-carboxamide; 1-(2-chloro-6-fluorophenyl)-4-((4-(piperidine-1-carbonyl)phenyl)amino)-1H-pyrazole-3-carboxamide; 1-(2,6-dichlorophenyl)-4-((4-(1-ethyl-1H-imidazol-2-yl)phenyl)amino)-1H-pyrazole-3-carboxamide; 1-(2,6-dichlorophenyl)-4-((4-(1-(2,2-difluoroethyl)-1H-imidazol-2-yl)phenyl)amino)-1H-pyrazole-3-carboxamide; 1-(2-chloro-6-fluorophenyl)-4-((4-(1-methyl-3-(trifluoromethyl)-1H-1,2,4-triazol-5-yl)phenyl)amino)-1H-pyrazole-3-carboxamide; 1-(2-chloro-6-fluorophenyl)-4-((4-(3-methyl-1H-pyrazol-1-yl)phenyl)amino)-1H-pyrazole-3-carboxamide; 1-(2-chloro-6-fluorophenyl)-4-((4-(5-methyl-1H-pyrazol-1-yl)phenyl)amino)-1H-pyrazole-3-carboxamide; 1-(2,6-dichlorophenyl)-4-((4-(3-ethylpyridin-2-yl)phenyl)amino)-1H-pyrazole-3-carboxamide; 1-(2,6-dichlorophenyl)-4-((5-(pyrrolidine-1-carbonyl)pyrimidin-2-yl)amino)-1H-pyrazole-3-carboxamide; 1-(2,6-dichlorophenyl)-4-((4-(3-(trifluoromethyl)-4,5,6,7-tetrahydro-2H-indazol-2-yl)phenyl)amino)-1H-pyrazole-3-carboxamide; 1-(2,6-dichlorophenyl)-4-((4-((1-methylpiperidin-4-yl)oxy)phenyl)amino)-1H-pyrazole-3-carboxamide; 1-(2,6-dichlorophenyl)-4-((4-(1-methyl-1H-tetrazol-5-yl)phenyl)amino)-1H-pyrazole-3-carboxamide; 1-(2,6-dichlorophenyl)-4-((4′-fluoro-[1,1′-biphenyl]-4-yl)amino)-1H-pyrazole-3-carboxamide; 1-(2,6-dichlorophenyl)-4-((4-(3-ethyl-1H-pyrazol-1-yl)phenyl)amino)-1H-pyrazole-3-carboxamide; 1-(2,6-dichlorophenyl)-4-((4-(5-ethyl-1H-pyrazol-1-yl)phenyl)amino)-1H-pyrazole-3-carboxamide; 1-(2,6-dichlorophenyl)-4-((4-(4-methyl-5-(trifluoromethyl)-4H-1,2,4-triazol-3-yl)phenyl)amino)-1H-pyrazole-3-carboxamide; 1-(2,6-dichlorophenyl)-4-((4-(5-methyl-1H-tetrazol-1-yl)phenyl)amino)-1H-pyrazole-3-carboxamide; 1-(2,6-dichlorophenyl)-4-((3′-fluoro-[1,1′-biphenyl]-4-yl)amino)-1H-pyrazole-3-carboxamide; 1-(2,6-dichlorophenyl)-4-((2′,5′-difluoro-[1,1′-biphenyl]-4-yl)amino)-1H-pyrazole-3-carboxamide; 1-(2,6-dichlorophenyl)-4-((4-(3-fluoropyrrolidine-1-carbonyl)phenyl)amino)-1H-pyrazole-3-carboxamide; 1-(2,6-dichlorophenyl)-4-((4-(3,3-difluoropyrrolidine-1-carbonyl)phenyl)amino)-1H-pyrazole-3-carboxamide; 1-(2,6-dichlorophenyl)-4-((4-(3-(dimethylamino)pyrrolidine-1-carbonyl)phenyl)amino)-1H-pyrazole-3-carboxamide; 4-((4-([1,3′-bipyrrolidine]-1′-carbonyl)phenyl)amino)-1-(2,6-dichlorophenyl)-1H-pyrazole-3-carboxamide; 1-(2,6-dichlorophenyl)-4-((4-(5-ethyl-4-methyl-1H-pyrazol-1-yl)phenyl)amino)-1H-pyrazole-3-carboxamide; 1-(2,6-dichlorophenyl)-4-((4-(1-(2-(dimethylamino)ethyl)-1H-imidazol-2-yl)phenyl)amino)-1H-pyrazole-3-carboxamide; 4-((4-(4-cyano-3-methyl-1H-pyrazol-1-yl)phenyl)amino)-1-(2,6-dichlorophenyl)-1H-pyrazole-3-carboxamide; 4-((4-(4-cyano-5-methyl-1H-pyrazol-1-yl)phenyl)amino)-1-(2,6-dichlorophenyl)-1H-pyrazole-3-carboxamide; 1-(2,6-dichlorophenyl)-4-((4-(3,3,4,4-tetrafluoropyrrolidine-1-carbonyl)phenyl)amino)-1H-pyrazole-3-carboxamide; 1-(2,6-dichlorophenyl)-4-((2′-fluoro-[1,1′-biphenyl]-4-yl)amino)-1H-pyrazole-3-carboxamide; 4-((2′-cyano-[1,1′-biphenyl]-4-yl)amino)-1-(2,6-dichlorophenyl)-1H-pyrazole-3-carboxamide; 1-(2,6-dichlorophenyl)-4-((4-(5-methyl-3-(trifluoromethyl)-1H-pyrazol-1-yl)phenyl)amino)-1H-pyrazole-3-carboxamide; 1-(2,6-dichlorophenyl)-4-((4-(3-methyl-5-(trifluoromethyl)-1H-pyrazol-1-yl)phenyl)amino)-1H-pyrazole-3-carboxamide; 1-(2,6-dichlorophenyl)-4-((4-(5-ethyl-1H-tetrazol-1-yl)phenyl)amino)-1H-pyrazole-3-carboxamide; 1-(2,6-dichlorophenyl)-4-((4-(3-methylpyridin-4-yl)phenyl)amino)-1H-pyrazole-3-carboxamide; 1-(2,6-dichlorophenyl)-4-((4-isopropoxyphenyl)amino)-1H-pyrazole-3-carboxamide; 1-(2,6-dichlorophenyl)-4-((4-(5-isopropyl-1H-pyrazol-1-yl)phenyl)amino)-1H-pyrazole-3-carboxamide; 1-(2,6-dichlorophenyl)-4-((4-(3-isopropyl-1H-pyrazol-1-yl)phenyl)amino)-1H-pyrazole-3-carboxamide; 1-(2,6-dichlorophenyl)-N-methyl-4-((4-(morpholine-4-carbonyl)phenyl)amino)-1H-pyrazole-3-carboxamide; 1-(2,6-dichlorophenyl)-4-((4-sulfamoylphenyl)amino)-1H-pyrazole-3-carboxamide; 4-((4-cyanophenyl)amino)-1-(2,6-dichlorophenyl)-1H-pyrazole-3-carboxamide; 4-(4-((3-carbamoyl-1-(2,6-dichlorophenyl)-1H-pyrazol-4-yl)amino)phenyl)-3-methylpyridine_1-oxide; 1-(2,6-dichlorophenyl)-4-((4-(1-(2,2,2-trifluoroethyl)-1H-imidazol-2-yl)phenyl)amino)-1H-pyrazole-3-carboxamide; 1-(2,6-dichlorophenyl)-4-((4-(7-methyl-5,6,7,8-tetrahydro-[1,2,4]triazolo[4,3-a]pyrazin-3-yl)phenyl)amino)-1H-pyrazole-3-carboxamide; 1-(2,6-dichlorophenyl)-4-((4-(ethylsulfonamido)phenyl)amino)-1H-pyrazole-3-carboxamide; 1-(2,6-dichlorophenyl)-4-((4-(methylsulfonamido)phenyl)amino)-1H-pyrazole-3-carboxamide; 1-(2,6-dichlorophenyl)-4-((4-(1,1-dioxidoisothiazolidin-2-yl)phenyl)amino)-1H-pyrazole-3-carboxamide; 1-(2,6-dichlorophenyl)-4-((4-(5-(2-(dimethylamino)ethyl)-1H-tetrazol-1-yl)phenyl)amino)-1H-pyrazole-3-carboxamide; 1-(2,6-dichlorophenyl)-4-((4-(5-isopropyl-3-(trifluoromethyl)-1H-1,2,4-triazol-1-yl)phenyl)amino)-1H-pyrazole-3-carboxamide; 1-(2,6-dichlorophenyl)-4-((4-(5-methyl-3-(trifluoromethyl)-1H-1,2,4-triazol-1-yl)phenyl)amino)-1H-pyrazole-3-carboxamide; 4-((4-(3-cyanoazetidine-1-carbonyl)phenyl)amino)-1-(2,6-dichlorophenyl)-1H-pyrazole-3-carboxamide; 1-(2,6-dichlorophenyl)-4-((4-(5-ethyl-3-(trifluoromethyl)-1H-1,2,4-triazol-1-yl)phenyl)amino)-1H-pyrazole-3-carboxamide; 1-(2,6-dichlorophenyl)-4-((4-(1-ethyl-3-(trifluoromethyl)-1H-1,2,4-triazol-5-yl)phenyl)amino)-1H-pyrazole-3-carboxamide; 1-(2,6-dichlorophenyl)-4-((4-(5-propyl-3-(trifluoromethyl)-1H-1,2,4-triazol-1-yl)phenyl)amino)-1H-pyrazole-3-carboxamide; 1-(2,6-dichlorophenyl)-4-((4-(1-isopropyl-3-(trifluoromethyl)-1H-1,2,4-triazol-5-yl)phenyl)amino)-1H-pyrazole-3-carboxamide; 1-(2,6-dichlorophenyl)-4-((4-(5-(methoxymethyl)-3-(trifluoromethyl)-1H-1,2,4-triazol-1-yl)phenyl)amino)-1H-pyrazole-3-carboxamide; 1-(2,6-dichlorophenyl)-4-((4-(1-propyl-3-(trifluoromethyl)-1H-1,2,4-triazol-5-yl)phenyl)amino)-1H-pyrazole-3-carboxamide; 1-(2-chloro-6-fluorophenyl)-4-((4-(5-ethyl-3-(trifluoromethyl)-1H-1,2,4-triazol-1-yl)phenyl)amino)-1H-pyrazole-3-carboxamide; 1-(2,6-difluorophenyl)-4-((4-(5-ethyl-3-(trifluoromethyl)-1H-1,2,4-triazol-1-yl)phenyl)amino)-1H-pyrazole-3-carboxamide; 1-(2,6-difluorophenyl)-4-((4-(1-methyl-3-(trifluoromethyl)-1H-1,2,4-triazol-5-yl)phenyl)amino)-1H-pyrazole-3-carboxamide; 1-(2-chloro-6-fluorophenyl)-4-((4-(5-ethyl-1H-pyrazol-1-yl)phenyl)amino)-1H-pyrazole-3-carboxamide; 1-(2,6-difluorophenyl)-4-((4-(5-ethyl-1H-pyrazol-1-yl)phenyl)amino)-1H-pyrazole-3-carboxamide; 1-(2,6-difluorophenyl)-4-((4-((1-methylpiperidin-4-yl)oxy)phenyl)amino)-1H-pyrazole-3-carboxamide; 1-(2-chloro-6-fluorophenyl)-4-((4-(1-ethyl-3-(trifluoromethyl)-1H-1,2,4-triazol-5-yl)phenyl)amino)-1H-pyrazole-3-carboxamide; 4-((3-carbamoyl-1-(2,6-dichlorophenyl)-1H-pyrazol-4-yl)amino)benzenesulfonic_acid; 1-(2,6-dichlorophenyl)-4-((4-(5-ethyl-1H-1,2,4-triazol-1-yl)phenyl)amino)-1H-pyrazole-3-carboxamide; 1-(2,6-dichlorophenyl)-4-((4-(1-(methoxymethyl)-4-(trifluoromethyl)-1H-imidazol-2-yl)phenyl)amino)-1H-pyrazole-3-carboxamide; 1-(2,6-dichlorophenyl)-4-((4-(1-ethyl-4-(trifluoromethyl)-1H-imidazol-2-yl)phenyl)amino)-1H-pyrazole-3-carboxamide; 1-(2,6-dichlorophenyl)-4-((4-(1-methyl-4-(trifluoromethyl)-1H-imidazol-2-yl)phenyl)amino)-1H-pyrazole-3-carboxamide; 1-(2-chloro-6-fluorophenyl)-4-((4-((1-methylpiperidin-4-yl)oxy)phenyl)amino)-1H-pyrazole-3-carboxamide; 1-(2,6-difluorophenyl)-4-((4-(4-methylpyridazin-3-yl)phenyl)amino)-1H-pyrazole-3-carboxamide; 1-(2,6-dichlorophenyl)-4-((4-(5-((dimethylamino)methyl)-3-(trifluoromethyl)-1H-1,2,4-triazol-1-yl)phenyl)amino)-1H-pyrazole-3-carboxamide; 1-(2-chloro-6-fluorophenyl)-4-((4-(5-(methoxymethyl)-3-(trifluoromethyl)-1H-1,2,4-triazol-1-yl)phenyl)amino)-1H-pyrazole-3-carboxamide; 1-(2,6-difluorophenyl)-4-((4-(5-(methoxymethyl)-3-(trifluoromethyl)-1H-1,2,4-triazol-1-yl)phenyl)amino)-1H-pyrazole-3-carboxamide; 1-(2,6-dichlorophenyl)-4-((4-(5-(methoxymethyl)-1H-1,2,4-triazol-1-yl)phenyl)amino)-1H-pyrazole-3-carboxamide; 1-(2,6-dichlorophenyl)-4-((4-(1-(oxetan-3-yl)-4-(trifluoromethyl)-1H-imidazol-2-yl)phenyl)amino)-1H-pyrazole-3-carboxamide; 1-(2,6-difluorophenyl)-4-((4-(1-ethyl-3-(trifluoromethyl)-1H-1,2,4-triazol-5-yl)phenyl)amino)-1H-pyrazole-3-carboxamide; 1-(2-chloro-6-fluorophenyl)-4-((4-(1-ethyl-4-(trifluoromethyl)-1H-imidazol-2-yl)phenyl)amino)-1H-pyrazole-3-carboxamide; 1-(2,6-difluorophenyl)-4-((4-(1-ethyl-4-(trifluoromethyl)-1H-imidazol-2-yl)phenyl)amino)-1H-pyrazole-3-carboxamide; 1-(2-chloro-6-fluorophenyl)-4-((4-(1-methyl-4-(trifluoromethyl)-1H-imidazol-2-yl)phenyl)amino)-1H-pyrazole-3-carboxamide; 1-(2,6-dichlorophenyl)-4-((4-(1-(2-(dimethylamino)ethyl)-4-(trifluoromethyl)-1H-imidazol-2-yl)phenyl)amino)-1H-pyrazole-3-carboxamide; 1-(2,6-dichlorophenyl)-4-((4-(1-(2-methoxyethyl)-4-(trifluoromethyl)-1H-imidazol-2-yl)phenyl)amino)-1H-pyrazole-3-carboxamide; 1-(2,6-difluorophenyl)-4-((4-(1-methyl-4-(trifluoromethyl)-1H-imidazol-2-yl)phenyl)amino)-1H-pyrazole-3-carboxamide; 1-(2,6-difluorophenyl)-4-((4-(1-isopropyl-1H-1,2,4-triazol-5-yl)phenyl)amino)-1H-pyrazole-3-carboxamide; 4-((4-(4-cyano-1-ethyl-1H-imidazol-2-yl)phenyl)amino)-1-(2,6-dichlorophenyl)-1H-pyrazole-3-carboxamide; 1-(2,6-dichlorophenyl)-4-((4-(1-propyl-4-(trifluoromethyl)-1H-imidazol-2-yl)phenyl)amino)-1H-pyrazole-3-carboxamide; 4-((4-(1-butyl-4-(trifluoromethyl)-1H-imidazol-2-yl)phenyl)amino)-1-(2,6-dichlorophenyl)-1H-pyrazole-3-carboxamide; (R)-1-(2,6-dichlorophenyl)-4-((4-(3-(dimethylamino)pyrrolidine-1-carbonyl)phenyl)amino)-1H-pyrazole-3-carboxamide; (S)-1-(2,6-dichlorophenyl)-4-((4-(3-(dimethylamino)pyrrolidine-1-carbonyl)phenyl)amino)-1H-pyrazole-3-carboxamide; (R)-1-(2,6-dichlorophenyl)-4-((4-(3-fluoropyrrolidine-1-carbonyl)phenyl)amino)-1H-pyrazole-3-carboxamide; (S)-1-(2,6-dichlorophenyl)-4-((4-(3-fluoropyrrolidine-1-carbonyl)phenyl)amino)-1H-pyrazole-3-carboxamide; 4-((4-(3-aminopyrrolidine-1-carbonyl)phenyl)amino)-1-(2,6-dichlorophenyl)-1H-pyrazole-3-carboxamide; (R)-4-((4-(3-aminopyrrolidine-1-carbonyl)phenyl)amino)-1-(2,6-dichlorophenyl)-1H-pyrazole-3-carboxamide; (S)-4-((4-(3-aminopyrrolidine-1-carbonyl)phenyl)amino)-1-(2,6-dichlorophenyl)-1H-pyrazole-3-carboxamide; 1-(2,6-dichlorophenyl)-4-((2-(2-methyl-1H-imidazol-1-yl)thiazol-4-yl)amino)-1H-pyrazole-3-carboxamide; 1-(2,6-dichlorophenyl)-4-((4-(pyridazin-4-yl)phenyl)amino)-1H-pyrazole-3-carboxamide; 1-(2,6-dichlorophenyl)-4-((4-(5-ethylpyridazin-4-yl)phenyl)amino)-1H-pyrazole-3-carboxamide; tert-butyl_(1-(4-((3-carbamoyl-1-(2,6-dichlorophenyl)-1H-pyrazol-4-yl)amino)benzoyl)pyrrolidin-3-yl)carbamate; (R)-tert-butyl_(1-(4-((3-carbamoyl-1-(2,6-dichlorophenyl)-1H-pyrazol-4-yl)amino)benzoyl)pyrrolidin-3-yl)carbamate; (S)-tert-butyl_(1-(4-((3-carbamoyl-1-(2,6-dichlorophenyl)-1H-pyrazol-4-yl)amino)benzoyl)pyrrolidin-3-yl)carbamate; 1-(2,6-dichlorophenyl)-4-((4-(5-methyl-1H-1,2,4-triazol-1-yl)phenyl)amino)-1H-pyrazole-3-carboxamide; 1-(2,6-dichlorophenyl)-4-((4-(5-(2-methoxyethyl)-1H-1,2,4-triazol-1-yl)phenyl)amino)-1H-pyrazole-3-carboxamide; 1-(2,6-dichlorophenyl)-4-((6-(5-(methoxymethyl)-3-(trifluoromethyl)-1H-1,2,4-triazol-1-yl)pyridin-3-yl)amino)-1H-pyrazole-3-carboxamide; 1-(2,6-dichlorophenyl)-4-((6-(5-ethyl-3-(trifluoromethyl)-1H-1,2,4-triazol-1-yl)pyridin-3-yl)amino)-1H-pyrazole-3-carboxamide; 1-(2,6-dichlorophenyl)-4-((4-(5-(2-methoxyethyl)-3-(trifluoromethyl)-1H-1,2,4-triazol-1-yl)phenyl)amino)-1H-pyrazole-3-carboxamide; (R)-4-((4-([1,3′-bipyrrolidine]-1′-carbonyl)phenyl)amino)-1-(2,6-dichlorophenyl)-1H-pyrazole-3-carboxamide; (S)-4-((4-([1,3′-bipyrrolidine]-1′-carbonyl)phenyl)amino)-1-(2,6-dichlorophenyl)-1H-pyrazole-3-carboxamide; 4-((4-([1,2,4]triazolo[4,3-a]pyridin-3-yl)phenyl)amino)-1-(2,6-dichlorophenyl)-1H-pyrazole-3-carboxamide; 1-(2,6-dichlorophenyl)-4-((4-(5-(trifluoromethyl)-1H-1,2,4-triazol-3-yl)phenyl)amino)-1H-pyrazole-3-carboxamide; 1-(2,6-dichlorophenyl)-4-((6-(5-(methoxymethyl)-3-(trifluoromethyl)-1H-1,2,4-triazol-1-yl)pyridazin-3-yl)amino)-1H-pyrazole-3-carboxamide; 1-(2,6-dichlorophenyl)-4-((6-(5-ethyl-3-(trifluoromethyl)-1H-1,2,4-triazol-1-yl)pyridazin-3-yl)amino)-1H-pyrazole-3-carboxamide; 4-((6-(1H-1,2,4-triazol-1-yl)pyridin-3-yl)amino)-1-(2,6-dichlorophenyl)-1H-pyrazole-3-carboxamide; 1-(2,6-dichlorophenyl)-4-((4-(1-(2-hydroxyethyl)-4-(trifluoromethyl)-1H-imidazol-2-yl)phenyl)amino)-1H-pyrazole-3-carboxamide; 1-(2,6-dichlorophenyl)-4-((6-(1-ethyl-4-(trifluoromethyl)-1H-imidazol-2-yl)pyridin-3-yl)amino)-1H-pyrazole-3-carboxamide; 1-(2,6-dichlorophenyl)-4-((4-(5-methylpyridazin-4-yl)phenyl)amino)-1H-pyrazole-3-carboxamide; 1-(2,6-dichlorophenyl)-4-((4-(5-isopropyl-1H-1,2,4-triazol-1-yl)phenyl)amino)-1H-pyrazole-3-carboxamide; 4-((4-(1-(1-acetylazetidin-3-yl)-4-(trifluoromethyl)-1H-imidazol-2-yl)phenyl)amino)-1-(2,6-dichlorophenyl)-1H-pyrazole-3-carboxamide; 1-(2,6-dichlorophenyl)-4-((6-(5-ethyl-1H-1,2,4-triazol-1-yl)pyridin-3-yl)amino)-1H-pyrazole-3-carboxamide; 1-(2,6-dichlorophenyl)-4-((6-(2-methoxyphenyl)pyridin-3-yl)amino)-1H-pyrazole-3-carboxamide; 1-(2,6-dichlorophenyl)-4-((6-(2-oxopyrrolidin-1-yl)pyridin-3-yl)amino)-1H-pyrazole-3-carboxamide; 1-(2,6-dichlorophenyl)-4-((5-methyl-6-(1H-pyrazol-1-yl)pyridin-3-yl)amino)-1H-pyrazole-3-carboxamide; 1-(2,6-dichlorophenyl)-4-((4-(5-(oxetan-3-yl)-1H-1,2,4-triazol-1-yl)phenyl)amino)-1H-pyrazole-3-carboxamide; 1-(2,6-dichlorophenyl)-4-((4-(5-(hydroxymethyl)-1H-1,2,4-triazol-1-yl)phenyl)amino)-1H-pyrazole-3-carboxamide; 4-((4-(4-cyano-1-(oxetan-3-yl)-1H-imidazol-2-yl)phenyl)amino)-1-(2,6-dichlorophenyl)-1H-pyrazole-3-carboxamide; 1-(2,6-dichlorophenyl)-4-((6-(5-methylpyridazin-4-yl)pyridin-3-yl)amino)-1H-pyrazole-3-carboxamide; 1-(2,6-dichlorophenyl)-4-((6-(5-ethylpyridazin-4-yl)pyridin-3-yl)amino)-1H-pyrazole-3-carboxamide; 1-(2,6-dichlorophenyl)-4-((6-(pyridazin-4-yl)pyridin-3-yl)amino)-1H-pyrazole-3-carboxamide; 1-(2,6-dichlorophenyl)-4-((6-oxo-1,6-dihydropyridin-3-yl)amino)-1H-pyrazole-3-carboxamide; (S)-1-(2,6-dichlorophenyl)-4-((6-(3-(dimethylamino)pyrrolidine-1-carbonyl)pyridin-3-yl)amino)-1H-pyrazole-3-carboxamide; (S)-1-(2,6-dichlorophenyl)-4-((6-(3-fluoropyrrolidine-1-carbonyl)pyridin-3-yl)amino)-1H-pyrazole-3-carboxamide; (R)-4-((6-([1,3′-bipyrrolidine]-1′-carbonyl)pyridin-3-yl)amino)-1-(2,6-dichlorophenyl)-1H-pyrazole-3-carboxamide; (S)-4-((6-(3-aminopyrrolidine-1-carbonyl)pyridin-3-yl)amino)-1-(2,6-dichlorophenyl)-1H-pyrazole-3-carboxamide; 1-(2,6-dichlorophenyl)-4-((6-(pyrrolidine-1-carbonyl)pyridin-3-yl)amino)-1H-pyrazole-3-carboxamide; 1-(2-chloro-6-fluorophenyl)-4-((1-methyl-1H-pyrazol-4-yl)amino)-1H-pyrazole-3-carboxamide; 1-(2,6-dichlorophenyl)-4-((6-(4-methyl-4H-1,2,4-triazol-3-yl)pyridin-3-yl)amino)-1H-pyrazole-3-carboxamide; 4-((4-(4-cyano-1-(2-hydroxyethyl)-1H-imidazol-2-yl)phenyl)amino)-1-(2,6-dichlorophenyl)-1H-pyrazole-3-carboxamide; 1-(2,6-dichlorophenyl)-4-((6-(3,3,4,4-tetrafluoropyrrolidine-1-carbonyl)pyridin-3-yl)amino)-1H-pyrazole-3-carboxamide; 1-(2,6-dichlorophenyl)-4-((6-(4-methylpiperazine-1-carbonyl)pyridin-3-yl)amino)-1H-pyrazole-3-carboxamide; 4-((6-(4-cyclopropylpiperazine-1-carbonyl)pyridin-3-yl)amino)-1-(2,6-dichlorophenyl)-1H-pyrazole-3-carboxamide; 1-(2,6-dichlorophenyl)-4-((6-(morpholine-4-carbonyl)pyridin-3-yl)amino)-1H-pyrazole-3-carboxamide; (S)-1-(2,6-dichlorophenyl)-4-((5-(3-(dimethylamino)pyrrolidine-1-carbonyl)pyridin-2-yl)amino)-1H-pyrazole-3-carboxamide; (S)-1-(2,6-dichlorophenyl)-4-((5-(3-fluoropyrrolidine-1-carbonyl)pyridin-2-yl)amino)-1H-pyrazole-3-carboxamide; (S)-4-((5-(3-aminopyrrolidine-1-carbonyl)pyridin-2-yl)amino)-1-(2,6-dichlorophenyl)-1H-pyrazole-3-carboxamide; 1-(2,6-dichlorophenyl)-4-((5-(3,3,4,4-tetrafluoropyrrolidine-1-carbonyl)pyridin-2-yl)amino)-1H-pyrazole-3-carboxamide; 1-(2,6-dichlorophenyl)-4-((5-(4-methylpiperazine-1-carbonyl)pyridin-2-yl)amino)-1H-pyrazole-3-carboxamide; 1-(2,6-dichlorophenyl)-4-((4-(5-(hydroxymethyl)-3-(trifluoromethyl)-1H-1,2,4-triazol-1-yl)phenyl)amino)-1H-pyrazole-3-carboxamide; 1-(2-chloro-6-ethoxyphenyl)-4-((1-methyl-1H-pyrazol-4-yl)amino)-1H-pyrazole-3-carboxamide; 1-(2,6-dichlorophenyl)-4-((6-(1-(2-hydroxyethyl)-4-(trifluoromethyl)-1H-imidazol-2-yl)pyridin-3-yl)amino)-1H-pyrazole-3-carboxamide; 1-(2,6-dichlorophenyl)-4-((1-(3,6-dimethylpyrazin-2-yl)-1H-pyrazol-4-yl)amino)-1H-pyrazole-3-carboxamide; 1-(2,6-dichlorophenyl)-4-((6-(1-methyl-1H-tetrazol-5-yl)pyridin-3-yl)amino)-1H-pyrazole-3-carboxamide; 1-(2,6-dichlorophenyl)-4-((4-(5-methyl-[1,2,4]triazolo[4,3-a]pyridin-3-yl)phenyl)amino)-1H-pyrazole-3-carboxamide; 1-(2,6-dichlorophenyl)-4-((1-methyl-1H-pyrazol-4-yl)amino)-1H-pyrazole-3-carboxamide; 1-(2,6-dichlorophenyl)-4-((1-isopropyl-1H-pyrazol-4-yl)amino)-1H-pyrazole-3-carboxamide; 1-(2,6-dichlorophenyl)-4-((2-methyl-2H-1,2,3-triazol-4-yl)amino)-1H-pyrazole-3-carboxamide; 1-(2,6-dichlorophenyl)-4-((1-methyl-1H-1,2,3-triazol-4-yl)amino)-1H-pyrazole-3-carboxamide; 1-(2,6-dichlorophenyl)-4-((1-methyl-2-oxo-1,2-dihydropyridin-4-yl)amino)-1H-pyrazole-3-carboxamide; (R)-4-((5-([1,3′-bipyrrolidine]-1′-carbonyl)pyridin-2-yl)amino)-1-(2,6-dichlorophenyl)-1H-pyrazole-3-carboxamide; 4-((5-(4-cyclopropylpiperazine-1-carbonyl)pyridin-2-yl)amino)-1-(2,6-dichlorophenyl)-1H-pyrazole-3-carboxamide; 1-(2,6-dichlorophenyl)-4-((6-(2-oxopiperidin-1-yl)pyridin-3-yl)amino)-1H-pyrazole-3-carboxamide; 1-(2,6-dichlorophenyl)-4-((6-(5-(hydroxymethyl)-3-(trifluoromethyl)-1H-1,2,4-triazol-1-yl)pyridin-3-yl)amino)-1H-pyrazole-3-carboxamide; 1-(2,6-dichlorophenyl)-4-((1-(3-fluoropyridin-2-yl)-1H-pyrazol-4-yl)amino)-1H-pyrazole-3-carboxamide; 4-((6-(4-cyano-1-(2-hydroxyethyl)-1H-imidazol-2-yl)pyridin-3-yl)amino)-1-(2,6-dichlorophenyl)-1H-pyrazole-3-carboxamide; 1-(2,6-dichlorophenyl)-4-((6-(5-methyl-[1,2,4]triazolo[4,3-a]pyridin-3-yl)pyridin-3-yl)amino)-1H-pyrazole-3-carboxamide; 1-(2,6-dichlorophenyl)-4-((1-methyl-6-oxo-1,6-dihydropyridin-3-yl)amino)-1H-pyrazole-3-carboxamide; 1-(2,6-dichlorophenyl)-4-((2-oxo-2H-[1,2′-bipyridin]-5′-yl)amino)-1H-pyrazole-3-carboxamide; 1-(2,6-dichlorophenyl)-4-((4-(5-methyl-1H-1,2,3-triazol-1-yl)phenyl)amino)-1H-pyrazole-3-carboxamide; 1-(2,6-dichlorophenyl)-4-((1-(4-methoxybenzyl)-1H-pyrazol-4-yl)amino)-1H-pyrazole-3-carboxamide; 1-(2,6-dichlorophenyl)-4-((6-(5-isopropyl-1H-tetrazol-1-yl)pyridin-3-yl)amino)-1H-pyrazole-3-carboxamide; 1-(2,6-dichlorophenyl)-4-((4′-methyl-[2,3′-bipyridin]-5-yl)amino)-1H-pyrazole-3-carboxamide; 1-(2,6-dichlorophenyl)-4-((3′-methyl-[2,2′-bipyridin]-5-yl)amino)-1H-pyrazole-3-carboxamide; 4-((1-(3-chloropyridin-2-yl)-1H-pyrazol-3-yl)amino)-1-(2,6-dichlorophenyl)-1H-pyrazole-3-carboxamide; 1-(2,6-dichlorophenyl)-4-((1-(pyridin-2-yl)-1H-pyrazol-3-yl)amino)-1H-pyrazole-3-carboxamide; 1-(2,6-dichlorophenyl)-4-((6-(3,5-dimethyl-1H-pyrazol-1-yl)pyridin-3-yl)amino)-1H-pyrazole-3-carboxamide; 1-(2,6-dichlorophenyl)-4-((1-(pyridin-2-yl)-1H-imidazol-4-yl)amino)-1H-pyrazole-3-carboxamide; 1-(2,6-dichlorophenyl)-4-((6-(4-isopropyl-4H-1,2,4-triazol-3-yl)pyridin-3-yl)amino)-1H-pyrazole-3-carboxamide; 1-(2,6-dichlorophenyl)-4-((5-(morpholine-4-carbonyl)pyrazin-2-yl)amino)-1H-pyrazole-3-carboxamide; 1-(2,6-dichlorophenyl)-4-((5-(4-methylpiperazine-1-carbonyl)pyrazin-2-yl)amino)-1H-pyrazole-3-carboxamide; (R)-1-(2,6-dichlorophenyl)-4-((5-(3-fluoropyrrolidine-1-carbonyl)pyrazin-2-yl)amino)-1H-pyrazole-3-carboxamide; 1-(2-chloro-6-fluorophenyl)-4-((1-methyl-1H-1,2,4-triazol-5-yl)amino)-1H-pyrazole-3-carboxamide; 1-(2,6-dichlorophenyl)-4-((6-((1-methylpiperidin-4-yl)oxy)pyridin-3-yl)amino)-1H-pyrazole-3-carboxamide; 1-(2,6-dichlorophenyl)-4-((1-methyl-1H-1,2,4-triazol-5-yl)amino)-1H-pyrazole-3-carboxamide; 4-((1H-pyrazol-4-yl)amino)-1-(2,6-dichlorophenyl)-1H-pyrazole-3-carboxamide; 1-(2,6-dichlorophenyl)-4-(imidazo[1,2-b]pyridazin-3-ylamino)-1H-pyrazole-3-carboxamide; 1-(2,6-dichlorophenyl)-4-(pyrazolo[1,5-a]pyrimidin-3-ylamino)-1H-pyrazole-3-carboxamide; 1-(2,6-dichlorophenyl)-4-((2-((1-methylpiperidin-4-yl)amino)pyrimidin-5-yl)amino)-1H-pyrazole-3-carboxamide; 1-(2,6-dichlorophenyl)-4-((4-(5-ethyl-1H-1,2,3-triazol-1-yl)phenyl)amino)-1H-pyrazole-3-carboxamide; 1-(2,6-dichloro-4-fluorophenyl)-4-((4-(morpholine-4-carbonyl)phenyl)amino)-1H-pyrazole-3-carboxamide; 1-(2,6-dichloro-4-fluorophenyl)-4-((4-(methyl(1-methylpiperidin-4-yl)carbamoyl)phenyl)amino)-1H-pyrazole-3-carboxamide; 1-(2,6-dichloro-4-fluorophenyl)-4-((4-(4-ethyl-4H-1,2,4-triazol-3-yl)phenyl)amino)-1H-pyrazole-3-carboxamide; (E)-1-(2,6-dichlorophenyl)-4-((4-(4-(2-fluorovinyl)-4H-1,2,4-triazol-3-yl)phenyl)amino)-1H-pyrazole-3-carboxamide; 1-(2,6-dichloro-3-fluorophenyl)-4-((4-(morpholine-4-carbonyl)phenyl)amino)-1H-pyrazole-3-carboxamide; 1-(2,6-dichloro-3-fluorophenyl)-4-((4-(methyl(1-methylpiperidin-4-yl)carbamoyl)phenyl)amino)-1H-pyrazole-3-carboxamide; 1-(2,6-dichloro-3-fluorophenyl)-4-((4-(4-ethyl-4H-1,2,4-triazol-3-yl)phenyl)amino)-1H-pyrazole-3-carboxamide; 1-(2,6-dichlorophenyl)-4-((4-((2-methoxyethyl)carbamoyl)phenyl)amino)-1H-pyrazole-3-carboxamide; 1-(2,6-dichlorophenyl)-4-((4-((3-methoxypropyl)carbamoyl)phenyl)amino)-1H-pyrazole-3-carboxamide; 1-(2,6-dichlorophenyl)-4-((4-((2-fluoroethyl)carbamoyl)phenyl)amino)-1H-pyrazole-3-carboxamide; 1-(2,6-dichlorophenyl)-4-((4-((2-(diethylamino)ethyl)carbamoyl)phenyl)amino)-1H-pyrazole-3-carboxamide; 1-(2,6-dichlorophenyl)-4-((4-(4-(dimethylamino)piperidine-1-carbonyl)phenyl)amino)-1H-pyrazole-3-carboxamide; 1-(2,6-dichlorophenyl)-4-((4-(piperidin-4-yloxy)phenyl)amino)-1H-pyrazole-3-carboxamide; 1-(2,6-dichlorophenyl)-4-((4-(3-fluoroazetidine-1-carbonyl)phenyl)amino)-1H-pyrazole-3-carboxamide; 1-(2,6-dichlorophenyl)-4-((4-(3-hydroxyazetidine-1-carbonyl)phenyl)amino)-1H-pyrazole-3-carboxamide; 1-(2,6-dichlorophenyl)-4-((6-(4-methyl-3H-indazol-3-ylidene)-1,6-dihydropyridin-3-yl)amino)-1H-pyrazole-3-carboxamide; 1-(2,6-dichlorophenyl)-4-((5-morpholinopyridin-2-yl)amino)-1H-pyrazole-3-carboxamide; 1-(2,6-dichlorophenyl)-4-((5-(5-methyl-[1,2,4]triazolo[4,3-a]pyridin-3-yl)pyridin-2-yl)amino)-1H-pyrazole-3-carboxamide; 1-(2,6-dichlorophenyl)-4-((5-(5-methyl-1H-1,2,4-triazol-1-yl)pyridin-2-yl)amino)-1H-pyrazole-3-carboxamide; 1-(2,6-dichlorophenyl)-4-((5-(1-ethyl-1H-1,2,4-triazol-5-yl)pyridin-2-yl)amino)-1H-pyrazole-3-carboxamide; 1-(2,6-dichlorophenyl)-4-((5-(5-methyl-1H-1,2,3-triazol-1-yl)pyridin-2-yl)amino)-1H-pyrazole-3-carboxamide; 1-(2,6-dichlorophenyl)-4-((5-(1-methyl-4-(trifluoromethyl)-1H-imidazol-2-yl)pyridin-2-yl)amino)-1H-pyrazole-3-carboxamide; 1-(2,6-dichlorophenyl)-4-((5-(2-methyl-2H-tetrazol-5-yl)pyridin-2-yl)amino)-1H-pyrazole-3-carboxamide; 1-(2,6-dichlorophenyl)-4-((5-(5-methyl-1H-tetrazol-1-yl)pyridin-2-yl)amino)-1H-pyrazole-3-carboxamide; 1-(2,6-dichlorophenyl)-4-((6-(5-methyl-[1,2,4]triazolo[4,3-a]pyrazin-3-yl)pyridin-3-yl)amino)-1H-pyrazole-3-carboxamide; 1-(2,6-dichlorophenyl)-4-((5-(5-(trifluoromethyl)-1H-1,2,3-triazol-1-yl)pyridin-2-yl)amino)-1H-pyrazole-3-carboxamide; 1-(2,6-dichlorophenyl)-4-((4-(5-(trifluoromethyl)-1H-1,2,3-triazol-1-yl)phenyl)amino)-1H-pyrazole-3-carboxamide; 1-(2,6-dichlorophenyl)-4-((5-(3-hydroxyazetidine-1-carbonyl)pyridin-2-yl)amino)-1H-pyrazole-3-carboxamide; 1-(2,6-dichlorophenyl)-4-((6-(2-morpholino-2-oxoethyl)pyridin-3-yl)amino)-1H-pyrazole-3-carboxamide; 1-(2,6-dichlorophenyl)-4-((6-(2-(3-fluoroazetidin-1-yl)-2-oxoethyl)pyridin-3-yl)amino)-1H-pyrazole-3-carboxamide; 1-(2,6-dichlorophenyl)-4-((5-(4-(dimethylamino)piperidine-1-carbonyl)pyridin-2-yl)amino)-1H-pyrazole-3-carboxamide; (R)-1-(2,6-dichlorophenyl)-4-((5-(3-fluoropyrrolidine-1-carbonyl)pyridin-2-yl)amino)-1H-pyrazole-3-carboxamide; 4-((5-(3-cyanoazetidine-1-carbonyl)pyridin-2-yl)amino)-1-(2,6-dichlorophenyl)-1H-pyrazole-3-carboxamide; 1-(2,6-dichlorophenyl)-4-((5-(1-methyl-1H-tetrazol-5-yl)pyridin-2-yl)amino)-1H-pyrazole-3-carboxamide; 1-(2,6-dichlorophenyl)-4-((6-(5-(trifluoromethyl)-1H-1,2,3-triazol-1-yl)pyridin-3-yl)amino)-1H-pyrazole-3-carboxamide; 4-((6-(5-chloro-[1,2,4]triazolo[4,3-a]pyrazin-3-yl)pyridin-3-yl)amino)-1-(2,6-dichlorophenyl)-1H-pyrazole-3-carboxamide; 1-(2,6-dichlorophenyl)-4-((5-(4-methyl-1,4-diazepane-1-carbonyl)pyridin-2-yl)amino)-1H-pyrazole-3-carboxamide; 1-(2,6-dichlorophenyl)-4-((5-(3-fluoroazetidine-1-carbonyl)pyridin-2-yl)amino)-1H-pyrazole-3-carboxamide; (R)-1-(2-cyano-6-fluorophenyl)-4-((4-(3-fluoropyrrolidine-1-carbonyl)phenyl)amino)-1H-pyrazole-3-carboxamide; 1-(2,6-dichlorophenyl)-4-((4-(1,1-dioxidothiomorpholine-4-carbonyl)phenyl)amino)-1H-pyrazole-3-carboxamide; 1-(2-cyano-6-fluorophenyl)-4-((4-(1,1-dioxidothiomorpholine-4-carbonyl)phenyl)amino)-1H-pyrazole-3-carboxamide; 4-[4-(3-Carbamoyl-azetidine-1-carbonyl)-phenylamino]-1-(2,6-dichloro-phenyl)-1H-pyrazole-3-carboxylic acid amide; 1-(2-cyano-6-fluorophenyl)-4-((4-(morpholine-4-carbonyl)phenyl)amino)-1H-pyrazole-3-carboxamide; 1-(2,6-dichlorophenyl)-4-((6-((1,1-dioxidothiomorpholino)methyl)pyridin-3-yl)amino)-1H-pyrazole-3-carboxamide; 1-(2,6-dichlorophenyl)-4-((4-(3,3-dimethylazetidine-1-carbonyl)phenyl)amino)-1H-pyrazole-3-carboxamide; 1-(2-chloro-6-cyanophenyl)-4-((4-(1,1-dioxidothiomorpholine-4-carbonyl)phenyl)amino)-1H-pyrazole-3-carboxamide; 1-(2-chloro-6-cyanophenyl)-4-((4-(morpholine-4-carbonyl)phenyl)amino)-1H-pyrazole-3-carboxamide; 1-(2-Chloro-6-cyano-phenyl)-4-[4-((R)-3-fluoro-pyrrolidine-1-carbonyl)-phenylamino]-1H-pyrazole-3-carboxylic acid amide; 1-(2,6-dichlorophenyl)-4-((6-(5-(1,1-difluoroethyl)-1H-tetrazol-1-yl)pyridin-3-yl)amino)-1H-pyrazole-3-carboxamide; 1-(2,6-dichlorophenyl)-4-((1-(2-morpholino-2-oxoethyl)-1H-pyrazol-4-yl)amino)-1H-pyrazole-3-carboxamide; methyl_2-(4-((3-carbamoyl-1-(2,6-dichlorophenyl)-1H-pyrazol-4-yl)amino)-1H-pyrazol-1-yl)acetate; 1-(2,6-dichlorophenyl)-4-((6-(1,1-dioxidothiomorpholine-4-carbonyl)pyridin-3-yl)amino)-1H-pyrazole-3-carboxamide; 1-(2,6-dichlorophenyl)-4-((6-(3,3-dimethylazetidine-1-carbonyl)pyridin-3-yl)amino)-1H-pyrazole-3-carboxamide; 1-(2,6-dichlorophenyl)-4-((5-(3,3-dimethylazetidine-1-carbonyl)pyridin-2-yl)amino)-1H-pyrazole-3-carboxamide; 1-(2,6-dichlorophenyl)-4-((4-(1-(methylimino)-1-oxidothiomorpholine-4-carbonyl)phenyl)amino)-1H-pyrazole-3-carboxamide; 1-(2,6-dichlorophenyl)-4-((4-(thiomorpholine-4-carbonyl)phenyl)amino)-1H-pyrazole-3-carboxamide; 1-(2,6-dichlorophenyl)-4-((4-(1-imino-1-oxidothiomorpholine-4-carbonyl)phenyl)amino)-1H-pyrazole-3-carboxamide; 1-(2,6-dichlorophenyl)-4-((1-(2-(1,1-dioxidothiomorpholino)-2-oxoethyl)-1H-pyrazol-4-yl)amino)-1H-pyrazole-3-carboxamide; 1-(2,6-dichlorophenyl)-4-((6-(5,7-dimethyl-[1,2,4]triazolo[4,3-c]pyrimidin-3-yl)pyridin-3-yl)amino)-1H-pyrazole-3-carboxamide; 1-(2,6-dichlorophenyl)-4-((6-(5-(trifluoromethyl)-[1,2,4]triazolo[4,3-a]pyridin-3-yl)pyridin-3-yl)amino)-1H-pyrazole-3-carboxamide; 1-(2,6-dichlorophenyl)-4-((6-(5-(trifluoromethyl)-[1,2,4]triazolo[4,3-a]pyrazin-3-yl)pyridin-3-yl)amino)-1H-pyrazole-3-carboxamide; 1-(2,6-dichlorophenyl)-4-((6-(5-methylimidazo[1,2-a]pyridin-3-yl)pyridin-3-yl)amino)-1H-pyrazole-3-carboxamide; 1-(2,6-dichlorophenyl)-4-((6-(5-methyl-[1,2,4]triazolo[4,3-c]pyrimidin-3-yl)pyridin-3-yl)amino)-1H-pyrazole-3-carboxamide; 1-(2,6-dichlorophenyl)-4-((6-(5-methylthiazolo[2,3-c][1,2,4]triazol-3-yl)pyridin-3-yl)amino)-1H-pyrazole-3-carboxamide; 4-((6-(5-cyano-[1,2,4]triazolo[4,3-a]pyridin-3-yl)pyridin-3-yl)amino)-1-(2,6-dichlorophenyl)-1H-pyrazole-3-carboxamide; 1-(2,6-dichloro-4-fluorophenyl)-4-((6-(5-methyl-[1,2,4]triazolo[4,3-a]pyridin-3-yl)pyridin-3-yl)amino)-1H-pyrazole-3-carboxamide; and 4-((4-(3-carbamoylazetidine-1-carbonyl)phenyl)amino)-1-(2,6-dichlorophenyl)-1H-pyrazole-3-carboxamide.

20. The compound of claim 1, wherein the compound is selected from the group consisting of 2-(2,6-Dichloro-phenyl)-5-[4-(morpholine-4-carbonyl)-phenylamino]-2H-[1,2,3]triazole-4-carboxylic acid amide; 2-(2,6-Dichloro-phenyl)-5-[4-(4-methyl-piperazine-1-carbonyl)-phenylamino]-2H-[1,2,3]triazole-4-carboxylic acid amide; 2-(2,6-Dichloro-phenyl)-5-[4-(1H-tetrazol-5-yl)-phenylamino]-2H-[1,2,3]triazole-4-carboxylic acid amide; 2-(2,6-Dichloro-phenyl)-5-[4-(1-methyl-1H-tetrazol-5-yl)-phenylamino]-2H-[1,2,3]triazole-4-carboxylic acid amide; 2-(2,6-Dichloro-phenyl)-5-[4-((S)-3-dimethylamino-pyrrolidine-1-carbonyl)-phenylamino]-2H-[1,2,3]triazole-4-carboxylic acid amide; 2-(2,6-Dichloro-phenyl)-5-[4-((R)-3-fluoro-pyrrolidine-1-carbonyl)-phenylamino]-2H-[1,2,3]triazole-4-carboxylic acid amide; 5-[4-(3-Cyano-azetidine-1-carbonyl)-phenylamino]-2-(2,6-dichloro-phenyl)-2H-[1,2,3]triazole-4-carboxylic acid amide; 2-(2,6-dichlorophenyl)-5-((4-(1-(2-hydroxyethyl)-4-(trifluoromethyl)-1H-imidazol-2-yl)phenyl)amino)-2H-1,2,3-triazole-4-carboxamide; 2-(2,6-Dichloro-phenyl)-5-[4-(5,6,7,8-tetrahydro-[1,2,4]triazolo[4,3-a]pyridin-3-yl)-phenylamino]-2H-[1,2,3]triazole-4-carboxylic acid amide; 2-(2,6-Dichloro-phenyl)-5-[4-(6,7-dihydro-4H-pyrano[4,3-c]pyrazol-1-yl)-phenylamino]-2H-[1,2,3]triazole-4-carboxylic acid amide; 2-(2,6-Dichloro-phenyl)-5-[4-(6,7-dihydro-4H-pyrano[4,3-c]pyrazol-2-yl)-phenylamino]-2H-[1,2,3]triazole-4-carboxylic acid amide; 2-(2,6-dichlorophenyl)-5-((4-(1-(2-hydroxyethyl)-4-(trifluoromethyl)-1H-imidazol-2-yl)phenyl)amino)-2H-1,2,3-triazole-4-carboxamide; 2-(2,6-dichlorophenyl)-5-((4-(4-(2,2-difluoroethyl)-4H-1,2,4-triazol-3-yl)phenyl)amino)-2H-1,2,3-triazole-4-carboxamide; 2-(2,6-dichlorophenyl)-5-((4-((1-methylpiperidin-4-yl)oxy)phenyl)amino)-2H-1,2,3-triazole-4-carboxamide; 2-(2,6-dichlorophenyl)-5-((4-(1-methyl-4-(trifluoromethyl)-1H-imidazol-2-yl)phenyl)amino)-2H-1,2,3-triazole-4-carboxamide; 2-(2,6-dichlorophenyl)-5-((4-(4-isopropyl-4H-1,2,4-triazol-3-yl)phenyl)amino)-2H-1,2,3-triazole-4-carboxamide; 2-(2,6-dichlorophenyl)-5-((4-(4-methyl-4H-1,2,4-triazol-3-yl)phenyl)amino)-2H-1,2,3-triazole-4-carboxamide; 2-(2,6-dichlorophenyl)-5-((4-(1-(2-methoxyethyl)-4-(trifluoromethyl)-1H-imidazol-2-yl)phenyl)amino)-2H-1,2,3-triazole-4-carboxamide; 2-(2,6-dichlorophenyl)-5-((4-((3-methoxypropyl)carbamoyl)phenyl)amino)-2H-1,2,3-triazole-4-carboxamide; 2-(2,6-dichlorophenyl)-5-((4-(piperidin-4-yloxy)phenyl)amino)-2H-1,2,3-triazole-4-carboxamide; 2-(2,6-dichlorophenyl)-5-((4-(5-ethyl-3-(trifluoromethyl)-1H-1,2,4-triazol-1-yl)phenyl)amino)-2H-1,2,3-triazole-4-carboxamide; 2-(2,6-dichlorophenyl)-5-((4-(4-(dimethylamino)piperidine-1-carbonyl)phenyl)amino)-2H-1,2,3-triazole-4-carboxamide; 2-(2,6-dichlorophenyl)-5-((4-((2-methoxyethyl)carbamoyl)phenyl)amino)-2H-1,2,3-triazole-4-carboxamide; 2-(2,6-dichlorophenyl)-5-((4-(3-hydroxyazetidine-1-carbonyl)phenyl)amino)-2H-1,2,3-triazole-4-carboxamide; 2-(2,6-dichlorophenyl)-5-((4-(4-isopropyl-4H-1,2,4-triazol-3-yl)phenyl)amino)-2H-1,2,3-triazole-4-carboxamide; 2-(2,6-dichlorophenyl)-5-((4-(5-ethyl-1H-1,2,3-triazol-1-yl)phenyl)amino)-2H-1,2,3-triazole-4-carboxamide; 2-(2,6-dichlorophenyl)-5-((5-(4-methylpiperazine-1-carbonyl)pyridin-2-yl)amino)-2H-1,2,3-triazole-4-carboxamide; 2-(2,6-dichlorophenyl)-5-((4-((2-fluoroethyl)carbamoyl)phenyl)amino)-2H-1,2,3-triazole-4-carboxamide; 2-(2,6-dichlorophenyl)-5-((4-(3-fluoroazetidine-1-carbonyl)phenyl)amino)-2H-1,2,3-triazole-4-carboxamide; (S)-2-(2,6-dichlorophenyl)-5-((5-(3-fluoropyrrolidine-1-carbonyl)pyridin-2-yl)amino)-2H-1,2,3-triazole-4-carboxamide; 2-(2,6-dichlorophenyl)-5-((4-(5-methyl-1H-1,2,3-triazol-1-yl)phenyl)amino)-2H-1,2,3-triazole-4-carboxamide; 2-(2,6-dichlorophenyl)-5-((5-(3,3,4,4-tetrafluoropyrrolidine-1-carbonyl)pyridin-2-yl)amino)-2H-1,2,3-triazole-4-carboxamide; (R)-2-(2,6-dichlorophenyl)-5-((5-(3-(dimethylamino)pyrrolidine-1-carbonyl)pyridin-2-yl)amino)-2H-1,2,3-triazole-4-carboxamide; 2-(2,6-dichlorophenyl)-5-((5-(5-methyl-1H-tetrazol-1-yl)pyridin-2-yl)amino)-2H-1,2,3-triazole-4-carboxamide; 2-(2,6-dichlorophenyl)-5-((5-(3-fluoroazetidine-1-carbonyl)pyridin-2-yl)amino)-2H-1,2,3-triazole-4-carboxamide; (R)-2-(2,6-dichlorophenyl)-5-((5-(3-fluoropyrrolidine-1-carbonyl)pyridin-2-yl)amino)-2H-1,2,3-triazole-4-carboxamide; 2-(2,6-dichlorophenyl)-5-((5-(pyrrolidine-1-carbonyl)pyridin-2-yl)amino)-2H-1,2,3-triazole-4-carboxamide; 2-(2,6-dichloro-3-fluorophenyl)-5-((4-(morpholine-4-carbonyl)phenyl)amino)-2H-1,2,3-triazole-4-carboxamide; 2-(2,6-dichlorophenyl)-5-((6-(5-methyl-[1,2,4]triazolo[4,3-a]pyridin-3-yl)pyridin-3-yl)amino)-2H-1,2,3-triazole-4-carboxamide; 2-(2,6-dichlorophenyl)-5-((5-(morpholine-4-carbonyl)pyridin-2-yl)amino)-2H-1,2,3-triazole-4-carboxamide; 2-(2,6-dichlorophenyl)-5-((5-(5-methyl-1H-1,2,3-triazol-1-yl)pyridin-2-yl)amino)-2H-1,2,3-triazole-4-carboxamide; 2-(2,6-dichlorophenyl)-5-((5-(1-ethyl-1H-1,2,4-triazol-5-yl)pyridin-2-yl)amino)-2H-1,2,3-triazole-4-carboxamide; 2-(2,6-dichlorophenyl)-5-((5-morpholinopyridin-2-yl)amino)-2H-1,2,3-triazole-4-carboxamide; 2-(2,6-dichlorophenyl)-5-((5-(1-methyl-4-(trifluoromethyl)-1H-imidazol-2-yl)pyridin-2-yl)amino)-2H-1,2,3-triazole-4-carboxamide; (R)-2-(2,6-dichloro-3-fluorophenyl)-5-((4-(3-fluoropyrrolidine-1-carbonyl)phenyl)amino)-2H-1,2,3-triazole-4-carboxamide; (R)-2-(2,6-dichloro-4-fluorophenyl)-5-((4-(3-fluoropyrrolidine-1-carbonyl)phenyl)amino)-2H-1,2,3-triazole-4-carboxamide; (R)-2-(2-chloro-3,6-difluorophenyl)-5-((4-(3-fluoropyrrolidine-1-carbonyl)phenyl)amino)-2H-1,2,3-triazole-4-carboxamide; (R)-2-(2,6-dichloro-4-cyanophenyl)-5-((4-(3-fluoropyrrolidine-1-carbonyl)phenyl)amino)-2H-1,2,3-triazole-4-carboxamide; 2-(2,6-dichlorophenyl)-5-((5-(4-(dimethylamino)piperidine-1-carbonyl)pyridin-2-yl)amino)-2H-1,2,3-triazole-4-carboxamide; 2-(2,6-dichlorophenyl)-5-((5-(4-methyl-1,4-diazepane-1-carbonyl)pyridin-2-yl)amino)-2H-1,2,3-triazole-4-carboxamide; 2-(2,6-dichlorophenyl)-5-((6-(2-morpholino-2-oxoethyl)pyridin-3-yl)amino)-2H-1,2,3-triazole-4-carboxamide; 2-(2,6-dichlorophenyl)-5-((6-(2-(3-fluoroazetidin-1-yl)-2-oxoethyl)pyridin-3-yl)amino)-2H-1,2,3-triazole-4-carboxamide; 5-((5-(3-cyanoazetidine-1-carbonyl)pyridin-2-yl)amino)-2-(2,6-dichlorophenyl)-2H-1,2,3-triazole-4-carboxamide; 2-(2,6-dichlorophenyl)-5-((5-(3-hydroxyazetidine-1-carbonyl)pyridin-2-yl)amino)-2H-1,2,3-triazole-4-carboxamide; 2-(2,6-dichlorophenyl)-5-((4-(5-(trifluoromethyl)-1H-1,2,3-triazol-1-yl)phenyl)amino)-2H-1,2,3-triazole-4-carboxamide; 2-(2,6-dichlorophenyl)-5-((5-(5-methyl-1H-1,2,4-triazol-1-yl)pyridin-2-yl)amino)-2H-1,2,3-triazole-4-carboxamide; 2-(2,6-dichlorophenyl)-5-((5-(5-(trifluoromethyl)-1H-1,2,3-triazol-1-yl)pyridin-2-yl)amino)-2H-1,2,3-triazole-4-carboxamide; 2-(2,6-dichlorophenyl)-5-((5-(1-methyl-1H-tetrazol-5-yl)pyridin-2-yl)amino)-2H-1,2,3-triazole-4-carboxamide; 2-(2,6-dichlorophenyl)-5-((6-(5-(trifluoromethyl)-1H-1,2,3-triazol-1-yl)pyridin-3-yl)amino)-2H-1,2,3-triazole-4-carboxamide; 2-(2,6-dichlorophenyl)-5-((5-(1,1-dioxidothiomorpholine-4-carbonyl)pyridin-2-yl)amino)-2H-1,2,3-triazole-4-carboxamide; 2-(2,6-dichlorophenyl)-5-((6-(5-methyl-[1,2,4]triazolo[4,3-a]pyrazin-3-yl)pyridin-3-yl)amino)-2H-1,2,3-triazole-4-carboxamide; (R)-2-(2-chloro-6-cyanophenyl)-5-((4-(3-fluoropyrrolidine-1-carbonyl)phenyl)amino)-2H-1,2,3-triazole-4-carboxamide; 2-(2,6-dichlorophenyl)-5-((4-(1,1-dioxidothiomorpholine-4-carbonyl)phenyl)amino)-2H-1,2,3-triazole-4-carboxamide; (R)-2-(2-cyano-6-fluorophenyl)-5-((4-(3-fluoropyrrolidine-1-carbonyl)phenyl)amino)-2H-1,2,3-triazole-4-carboxamide; 2-(2,6-dichlorophenyl)-5-((5-(3,3-dimethylazetidine-1-carbonyl)pyridin-2-yl)amino)-2H-1,2,3-triazole-4-carboxamide; 2-(2,6-dichlorophenyl)-5-((4-(3,3-dimethylazetidine-1-carbonyl)phenyl)amino)-2H-1,2,3-triazole-4-carboxamide; (R)-2-(6-chloro-2,3-difluorophenyl)-5-((4-(3-fluoropyrrolidine-1-carbonyl)phenyl)amino)-2H-1,2,3-triazole-4-carboxamide; 2-(2,6-dichlorophenyl)-5-((1-(2-morpholino-2-oxoethyl)-1H-pyrazol-4-yl)amino)-2H-1,2,3-triazole-4-carboxamide; 2-(2,6-dichlorophenyl)-5-((4-(1-imino-1-oxidothiomorpholine-4-carbonyl)phenyl)amino)-2H-1,2,3-triazole-4-carboxamide; 2-(2,6-dichlorophenyl)-5-((4-(thiomorpholine-4-carbonyl)phenyl)amino)-2H-1,2,3-triazole-4-carboxamide; 2-(2,6-dichlorophenyl)-5-((4-(1-(methylimino)-1-oxidothiomorpholine-4-carbonyl)phenyl)amino)-2H-1,2,3-triazole-4-carboxamide; 2-(2,6-dichlorophenyl)-5-((1-(methylsulfonyl)-1H-pyrazol-4-yl)amino)-2H-1,2,3-triazole-4-carboxamide; 2-(2,6-dichlorophenyl)-5-((1-(ethylsulfonyl)-1H-pyrazol-4-yl)amino)-2H-1,2,3-triazole-4-carboxamide; 2-(2,6-dichlorophenyl)-5-((1-(2-(1,1-dioxidothiomorpholino)-2-oxoethyl)-1H-pyrazol-4-yl)amino)-2H-1,2,3-triazole-4-carboxamide; 2-(2,6-dichloro-4-fluorophenyl)-5-((4-(1,1-dioxidothiomorpholine-4-carbonyl)phenyl)amino)-2H-1,2,3-triazole-4-carboxamide; 2-(2,6-dichloro-4-methoxyphenyl)-5-((4-(thiomorpholine-4-carbonyl)phenyl)amino)-2H-1,2,3-triazole-4-carboxamide; and 2-(2,6-dichloro-4-fluorophenyl)-5-((4-(thiomorpholine-4-carbonyl)phenyl)amino)-2H-1,2,3-triazole-4-carboxamide.

21. A method of modulating the activity of a kinase, the method comprising contacting cells containing a kinase with a compound of Formula (II): 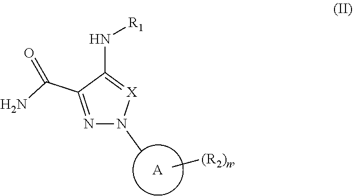
wherein:
X is N or CH;
R₁ is C(O)CH₂R₃, optionally substituted cycloalkyl, optionally substituted cycloheteroalkyl, optionally substituted cycloalkenyl, optionally substituted cycloheteroalkenyl, optionally substituted aryl, or optionally substituted heteroaryl;
each instance of R₂ is independently halo, haloalkyl, cyano, optionally substituted alkoxyl, optionally substituted alkyl, optionally substituted heteroalkyl, optionally substituted alkenyl, optionally substituted heteroalkenyl;
R₃ is optionally substituted cycloalkyl, optionally substituted cycloheteroalkyl, optionally substituted cycloalkenyl, optionally substituted cycloheteroalkenyl, optionally substituted aryl, or optionally substituted heteroaryl;
A is cycloalkyl, cycloheteroalkyl, cycloalkenyl, cycloheteroalkenyl, aryl, or heteroaryl; and
n is 0-5.

22. The method of claim 21, wherein X is N.

23. The method of claim 21, wherein X is CH.

24. The method of claim 21, wherein A is aryl or heteroaryl.

25. The method of claim 24, wherein each instance of R₂ is halo or cyano.

26. The method of claim 25, wherein n is 2 or 3.

27. The compound of claim 26, wherein the compound is represented by Formula (IIa):

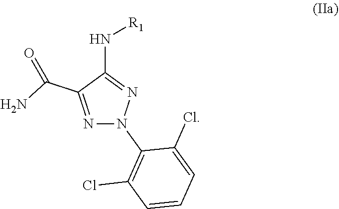

28. The method of claim 26, wherein the compound is represented by Formula (I):

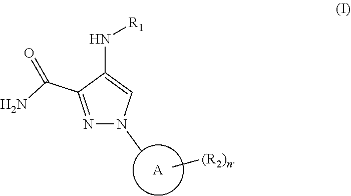

29. The method of claim 21, wherein R₁ is an optionally substituted aryl.

30. The method of claim 29, wherein R₁ is a substituted phenyl.

31. The method of claim 21, wherein R₁ is optionally substituted aryl or optionally substituted heteroaryl;

32. The method of claim 31, wherein R₁ is an optionally substituted aryl other than phenyl.

33. The method of claim 31, wherein R₁ is an optionally substituted heteroaryl.

34. The method of claim 31, wherein A is aryl or heteroaryl.

35. The method of claim 34, wherein A is aryl.

36. The method of claim 34, wherein A is heteroaryl.

37. The method of claim 34, wherein each instance of R₂ is halo or cyano.

38. The method of claim 37, wherein n is 2 or 3.

39. The method of claim 21, wherein the compound is selected from the group consisting of 1-(4-methoxyphenyl)-4-(2-morpholinoacetamido)-1H-pyrazole-3-carboxamide; 1-(2,6-difluorophenyl)-4-(2-morpholinoacetamido)-1H-pyrazole-3-carboxamide; 1-(2-fluorophenyl)-4-(2-morpholinoacetamido)-1H-pyrazole-3-carboxamide; 1-(2,6-difluorophenyl)-4-((6-methylpyrimidin-4-yl)amino)-1H-pyrazole-3-carboxamide; 1-(2-fluorophenyl)-4-((6-methylpyrimidin-4-yl)amino)-1H-pyrazole-3-carboxamide; 4-((5-aminopyridin-2-yl)amino)-1-(2,6-difluorophenyl)-1H-pyrazole-3-carboxamide; 1-(2,6-difluorophenyl)-4-(pyridin-2-ylamino)-1H-pyrazole-3-carboxamide; 4-((2-amino-6-methylpyrimidin-4-yl)amino)-1-(2,6-difluorophenyl)-1H-pyrazole-3-carboxamide; 4-(benzo[d]thiazol-2-ylamino)-1-(2,6-difluorophenyl)-1H-pyrazole-3-carboxamide; 1-(2,6-difluorophenyl)-4-((4-methylpyridin-2-yl)amino)-1H-pyrazole-3-carboxamide; 4-((1H-benzo[d]imidazol-2-yl)amino)-1-(2,6-difluorophenyl)-1H-pyrazole-3-carboxamide; 1-(2,6-difluorophenyl)-4-((4-(morpholine-4-carbonyl)phenyl)amino)-1H-pyrazole-3-carboxamide; 1-(2,6-difluorophenyl)-4-((4-((tetrahydro-2H-pyran-4-yl)carbamoyl)phenyl)amino)-1H-pyrazole-3-carboxamide; 1-(2,6-difluorophenyl)-4-((4-((2,2,2-trifluoroethyl)carbamoyl)phenyl)amino)-1H-pyrazole-3-carboxamide; 4-((4-(tert-butylcarbamoyl)phenyl)amino)-1-(2,6-difluorophenyl)-1H-pyrazole-3-carboxamide; 1-(2,6-difluorophenyl)-4-((4-(ethylcarbamoyl)phenyl)amino)-1H-pyrazole-3-carboxamide; 1-(2,6-dichlorophenyl)-4-((4-(morpholine-4-carbonyl)phenyl)amino)-1H-pyrazole-3-carboxamide; 1-(2,6-difluorophenyl)-4-((3-(morpholine-4-carbonyl)phenyl)amino)-1H-pyrazole-3-carboxamide; 4-((4-chlorophenyl)amino)-1-(2,6-difluorophenyl)-1H-pyrazole-3-carboxamide; 4-((4-chlorophenyl)amino)-1-(2,6-dichlorophenyl)-1H-pyrazole-3-carboxamide; 1-(2,6-dichlorophenyl)-4-(phenylamino)-1H-pyrazole-3-carboxamide; 1-(2-chloro-6-fluorophenyl)-4-((4-(morpholine-4-carbonyl)phenyl)amino)-1H-pyrazole-3-carboxamide; N-(tert-butyl)-5-((3-carbamoyl-1-(2,6-difluorophenyl)-1H-pyrazol-4-yl)amino)picolinamide; 5-((3-carbamoyl-1-(2,6-difluorophenyl)-1H-pyrazol-4-yl)amino)-N-ethylpicolinamide; 5-((3-carbamoyl-1-(2-fluoro-6-methoxyphenyl)-1H-pyrazol-4-yl)amino)-N-ethylpicolinamide; 4-((4-(tert-butylcarbamoyl)phenyl)amino)-1-(2,6-dichlorophenyl)-1H-pyrazole-3-carboxamide; 4-((4-(tert-butylcarbamoyl)phenyl)amino)-1-(2-chloro-6-fluorophenyl)-1H-pyrazole-3-carboxamide; 4-((4-(tert-butylcarbamoyl)-3-fluorophenyl)amino)-1-(2,6-dichlorophenyl)-1H-pyrazole-3-carboxamide; 4-((4-(tert-butylcarbamoyl)-3-fluorophenyl)amino)-1-(2-chloro-6-fluorophenyl)-1H-pyrazole-3-carboxamide; 1-(2,6-difluorophenyl)-4-((6-(morpholine-4-carbonyl)pyridin-3-yl)amino)-1H-pyrazole-3-carboxamide; 1-(2,6-dichlorophenyl)-4-((4-((1-methylpiperidin-4-yl)carbamoyl)phenyl)amino)-1H-pyrazole-3-carboxamide; 1-(2,6-dichlorophenyl)-4-((4-(4-methylpiperazine-1-carbonyl)phenyl)amino)-1H-pyrazole-3-carboxamide; tert-butyl_3-(4-((3-carbamoyl-1-(2,6-dichlorophenyl)-1H-pyrazol-4-yl)amino)benzamido)piperidine-1-carboxylate; 1-(2,6-dichlorophenyl)-4-((4-(ethylcarbamoyl)phenyl)amino)-1H-pyrazole-3-carboxamide; 1-(2-chloro-6-fluorophenyl)-4-((4-((1-methylpiperidin-4-yl)carbamoyl)phenyl)amino)-1H-pyrazole-3-carboxamide; 1-(2-chloro-6-fluorophenyl)-4-((4-(4-methylpiperazine-1-carbonyl)phenyl)amino)-1H-pyrazole-3-carboxamide; tert-butyl_3-(4-((3-carbamoyl-1-(2-chloro-6-fluorophenyl)-1H-pyrazol-4-yl)amino)benzamido)piperidine-1-carboxylate; 1-(2-chloro-6-fluorophenyl)-4-((4-(piperidin-3-ylcarbamoyl)phenyl)amino)-1H-pyrazole-3-carboxamide; 1-(2,6-dichlorophenyl)-4-((4-(piperidin-3-ylcarbamoyl)phenyl)amino)-1H-pyrazole-3-carboxamide; 1-(2-chloro-6-fluorophenyl)-4-((4-(ethylcarbamoyl)phenyl)amino)-1H-pyrazole-3-carboxamide; 1-(2,6-dichlorophenyl)-4-((4-pivaloylphenyl)amino)-1H-pyrazole-3-carboxamide; 1-(2-chloro-6-fluorophenyl)-4-((4-pivaloylphenyl)amino)-1H-pyrazole-3-carboxamide; 1-(2,6-dichlorophenyl)-4-((1,2-dimethyl-1H-benzo[d]imidazol-5-yl)amino)-1H-pyrazole-3-carboxamide; 1-(2-chloro-6-fluorophenyl)-4-((1,2-dimethyl-1H-benzo[d]imidazol-5-yl)amino)-1H-pyrazole-3-carboxamide; 1-(2,6-dichlorophenyl)-4-((4-(4-methylpyridazin-3-yl)phenyl)amino)-1H-pyrazole-3-carboxamide; 4-((4-(2-azaspiro[3.3]heptane-2-carbonyl)phenyl)amino)-1-(2,6-dichlorophenyl)-1H-pyrazole-3-carboxamide; 1-(2,6-dichlorophenyl)-4-((4-(1-ethyl-1H-1,2,4-triazol-5-yl)phenyl)amino)-1H-pyrazole-3-carboxamide; 1-(2,6-dichlorophenyl)-4-((4-(1-methyl-1H-1,2,3-triazol-5-yl)phenyl)amino)-1H-pyrazole-3-carboxamide; 1-(2,6-dichlorophenyl)-4-((4-(pyridazin-3-yl)phenyl)amino)-1H-pyrazole-3-carboxamide; 1-(2,6-dichlorophenyl)-4-((4-(methyl(1-methylpiperidin-4-yl)carbamoyl)phenyl)amino)-1H-pyrazole-3-carboxamide; 1-(2,6-dichlorophenyl)-4-((4-(5-methylpyrimidin-4-yl)phenyl)amino)-1H-pyrazole-3-carboxamide; 1-(2,6-dichlorophenyl)-4-((4-(5-ethylpyrimidin-4-yl)phenyl)amino)-1H-pyrazole-3-carboxamide; 1-(2,6-dichlorophenyl)-4-((4-(1,4-dimethyl-1H-imidazol-5-yl)phenyl)amino)-1H-pyrazole-3-carboxamide; 4-((4-(2-azaspiro[3.3]heptane-2-carbonyl)phenyl)amino)-1-(2-chloro-6-fluorophenyl)-1H-pyrazole-3-carboxamide; 1-(2,6-dichlorophenyl)-4-((4-(5,6,7,8-tetrahydro-[1,2,4]triazolo[4,3-a]pyridin-3-yl)phenyl)amino)-1H-pyrazole-3-carboxamide; 1-(2-chloro-6-fluorophenyl)-4-((4-(4-methylpyridazin-3-yl)phenyl)amino)-1H-pyrazole-3-carboxamide; 1-(2,6-dichlorophenyl)-4-((4-(6,8-dihydro-5H-[1,2,4]triazolo[3,4-c][1,4]oxazin-3-yl)phenyl)amino)-1H-pyrazole-3-carboxamide; 1-(2-chloro-6-fluorophenyl)-4-((4-(5,6,7,8-tetrahydro-[1,2,4]triazolo[4,3-a]pyridin-3-yl)phenyl)amino)-1H-pyrazole-3-carboxamide; 4-((4-(3-cyanopyridin-2-yl)phenyl)amino)-1-(2,6-dichlorophenyl)-1H-pyrazole-3-carboxamide; 4-((4-(4-cyanopyridazin-3-yl)phenyl)amino)-1-(2,6-dichlorophenyl)-1H-pyrazole-3-carboxamide; 4-((4-(2-azaspiro[3.3]heptane-2-carbonyl)phenyl)amino)-1-(2-fluoro-6-(trifluoromethyl)phenyl)-1H-pyrazole-3-carboxamide; 1-(2-chloro-6-fluorophenyl)-4-((4-(6,8-dihydro-5H-[1,2,4]triazolo[3,4-c][1,4]oxazin-3-yl)phenyl)amino)-1H-pyrazole-3-carboxamide; 1-(2,6-dichlorophenyl)-4-((4-(6,7,8,9-tetrahydro-5H-[1,2,4]triazolo[4,3-a]azepin-3-yl)phenyl)amino)-1H-pyrazole-3-carboxamide; 1-(2,6-difluorophenyl)-4-((4-(dimethylphosphoryl)phenyl)amino)-1H-pyrazole-3-carboxamide; 4-((4-(1H-indazol-1-yl)phenyl)amino)-1-(2,6-dichlorophenyl)-1H-pyrazole-3-carboxamide; 4-((4-(2H-indazol-2-yl)phenyl)amino)-1-(2,6-dichlorophenyl)-1H-pyrazole-3-carboxamide; 4-((4-(4-chloropyridazin-3-yl)phenyl)amino)-1-(2,6-dichlorophenyl)-1H-pyrazole-3-carboxamide; 1-(2-chloro-6-fluorophenyl)-4-((5-(morpholine-4-carbonyl)pyridin-2-yl)amino)-1H-pyrazole-3-carboxamide; 1-(2,6-dichlorophenyl)-4-((4-(4-(trifluoromethyl)pyridazin-3-yl)phenyl)amino)-1H-pyrazole-3-carboxamide; 1-(2,6-dichlorophenyl)-4-((4-(6,7-dihydropyrano[4,3-c]pyrazol-1(4H)-yl)phenyl)amino)-1H-pyrazole-3-carboxamide; 1-(2,6-dichlorophenyl)-4-((4-(6,7-dihydropyrano[4,3-c]pyrazol-2(4H)-yl)phenyl)amino)-1H-pyrazole-3-carboxamide; 1-(2,6-dichlorophenyl)-4-((4-(3-methyl-6-oxopyridazin-1(6H)-yl)phenyl)amino)-1H-pyrazole-3-carboxamide; 1-(2,6-dichlorophenyl)-4-((4-(4,5,6,7-tetrahydro-1H-benzo[d][1,2,3]triazol-1-yl)phenyl)amino)-1H-pyrazole-3-carboxamide; 1-(2,6-dichlorophenyl)-4-((5-(morpholine-4-carbonyl)pyridin-2-yl)amino)-1H-pyrazole-3-carboxamide; 1-(2,6-dichlorophenyl)-4-((4-(3-methyl-1H-pyrazol-1-yl)phenyl)amino)-1H-pyrazole-3-carboxamide; 4-((4-(3-chloro-1H-pyrazol-1-yl)phenyl)amino)-1-(2,6-dichlorophenyl)-1H-pyrazole-3-carboxamide; 1-(2,6-dichlorophenyl)-4-((4-(2-methyl-5-oxo-2,5-dihydro-1H-pyrazol-1-yl)phenyl)amino)-1H-pyrazole-3-carboxamide; 1-(2,6-dichlorophenyl)-4-((4-((1-methyl-1H-pyrazol-3-yl)oxy)phenyl)amino)-1H-pyrazole-3-carboxamide; 4-((4-(3-chloro-1H-1,2,4-triazol-1-yl)phenyl)amino)-1-(2,6-dichlorophenyl)-1H-pyrazole-3-carboxamide; 1-(2,6-dichlorophenyl)-4-((4-(5-methyl-1H-pyrazol-1-yl)phenyl)amino)-1H-pyrazole-3-carboxamide; 4-((4-(1H-benzo[d][1,2,3]triazol-1-yl)phenyl)amino)-1-(2,6-dichlorophenyl)-1H-pyrazole-3-carboxamide; 1-(2,6-dichlorophenyl)-4-((4-(piperidine-1-carbonyl)phenyl)amino)-1H-pyrazole-3-carboxamide; 1-(2,6-dichlorophenyl)-4-((4-(4-(2,2-difluoroethyl)piperazine-1-carbonyl)phenyl)amino)-1H-pyrazole-3-carboxamide; 4-((4-(4-cyclopropylpiperazine-1-carbonyl)phenyl)amino)-1-(2,6-dichlorophenyl)-1H-pyrazole-3-carboxamide; 1-(2,6-dichlorophenyl)-4-((4-((4-methylpiperazin-1-yl)sulfonyl)phenyl)amino)-1H-pyrazole-3-carboxamide; 4-((4-(4-(cyclopropylmethyl)piperazine-1-carbonyl)phenyl)amino)-1-(2,6-dichlorophenyl)-1H-pyrazole-3-carboxamide; 1-(2,6-dichlorophenyl)-4-((4-(4-ethyl-4H-1,2,4-triazol-3-yl)phenyl)amino)-1H-pyrazole-3-carboxamide; 1-(2,6-dichlorophenyl)-4-((4-(4-methyl-4H-1,2,4-triazol-3-yl)phenyl)amino)-1H-pyrazole-3-carboxamide; 1-(2,6-dichlorophenyl)-4-((4-(N-(1-methylpiperidin-4-yl)sulfamoyl)phenyl)amino)-1H-pyrazole-3-carboxamide; 1-(2,6-dichlorophenyl)-4-((4-(1-isopropyl-1H-1,2,4-triazol-5-yl)phenyl)amino)-1H-pyrazole-3-carboxamide; 1-(2,6-dichlorophenyl)-4-((4-(4-methyl-1,4-diazepane-1-carbonyl)phenyl)amino)-1H-pyrazole-3-carboxamide; 4-((4-(azetidine-1-carbonyl)phenyl)amino)-1-(2,6-dichlorophenyl)-1H-pyrazole-3-carboxamide; 1-(2,6-dichlorophenyl)-4-((4-(4-(2,2,2-trifluoroethyl)piperazine-1-carbonyl)phenyl)amino)-1H-pyrazole-3-carboxamide; 1-(2,6-dichlorophenyl)-4-((4-(pyrrolidine-1-carbonyl)phenyl)amino)-1H-pyrazole-3-carboxamide; 4-((4-(azepane-1-carbonyl)phenyl)amino)-1-(2,6-dichlorophenyl)-1H-pyrazole-3-carboxamide; 1-(2,6-dichlorophenyl)-4-((4-(1-methyl-1H-1,2,4-triazol-5-yl)phenyl)amino)-1H-pyrazole-3-carboxamide; 1-(2,6-dichlorophenyl)-4-((4-(2-oxobenzo[d]thiazol-3(2H)-yl)phenyl)amino)-1H-pyrazole-3-carboxamide; 4-((4-(4-cyclopentyl-4H-1,2,4-triazol-3-yl)phenyl)amino)-1-(2,6-dichlorophenyl)-1H-pyrazole-3-carboxamide; 1-(2,6-dichlorophenyl)-4-((4-(4-isobutyl-4H-1,2,4-triazol-3-yl)phenyl)amino)-1H-pyrazole-3-carboxamide; 1-(2,6-dichlorophenyl)-4-((4-(1-methyl-1H-imidazol-5-yl)phenyl)amino)-1H-pyrazole-3-carboxamide; 1-(2,6-dichlorophenyl)-4-((4-(4-isopropyl-4H-1,2,4-triazol-3-yl)phenyl)amino)-1H-pyrazole-3-carboxamide; 4-((4-(4-cyclopropyl-4H-1,2,4-triazol-3-yl)phenyl)amino)-1-(2,6-dichlorophenyl)-1H-pyrazole-3-carboxamide; 1-(2-chloro-6-fluorophenyl)-4-((4-(4-ethyl-4H-1,2,4-triazol-3-yl)phenyl)amino)-1H-pyrazole-3-carboxamide; 1-(2-chloro-6-fluorophenyl)-4-((4-(6,7-dihydropyrano[4,3-c]pyrazol-1(4H)-yl)phenyl)amino)-1H-pyrazole-3-carboxamide; 1-(2-chloro-6-fluorophenyl)-4-((4-(6,7-dihydropyrano[4,3-c]pyrazol-2(4H)-yl)phenyl)amino)-1H-pyrazole-3-carboxamide; 1-(2,6-dichlorophenyl)-4-((4-(4-(2,2,2-trifluoroethyl)-4H-1,2,4-triazol-3-yl)phenyl)amino)-1H-pyrazole-3-carboxamide; 1-(2-chloro-6-fluorophenyl)-4-((4-(1-isopropyl-1H-1,2,4-triazol-5-yl)phenyl)amino)-1H-pyrazole-3-carboxamide; 1-(2,6-dichlorophenyl)-4-((4-(4-(2,2-difluoroethyl)-4H-1,2,4-triazol-3-yl)phenyl)amino)-1H-pyrazole-3-carboxamide; 1-(2,6-dichlorophenyl)-4-((4-(4,5-dimethylpyridazin-3-yl)phenyl)amino)-1H-pyrazole-3-carboxamide; 1-(2,6-dichlorophenyl)-4-((4-(4-(2-(dimethylamino)ethyl)-4H-1,2,4-triazol-3-yl)phenyl)amino)-1H-pyrazole-3-carboxamide; 1-(2,6-dichlorophenyl)-4-((4-(1-methyl-3-(trifluoromethyl)-1H-1,2,4-triazol-5-yl)phenyl)amino)-1H-pyrazole-3-carboxamide; 1-(2,6-dichlorophenyl)-4-((4-(3,5-dimethyl-1H-pyrazol-1-yl)phenyl)amino)-1H-pyrazole-3-carboxamide; 1-(2,6-dichlorophenyl)-4-((4-(3,4-dimethyl-1H-pyrazol-1-yl)phenyl)amino)-1H-pyrazole-3-carboxamide; 1-(2,6-dichlorophenyl)-4-((4-(4,5-dimethyl-1H-pyrazol-1-yl)phenyl)amino)-1H-pyrazole-3-carboxamide; 4-((4-(4-chloro-3,5-dimethyl-1H-pyrazol-1-yl)phenyl)amino)-1-(2,6-dichlorophenyl)-1H-pyrazole-3-carboxamide; 4-((4-(4-chloro-5-methyl-1H-pyrazol-1-yl)phenyl)amino)-1-(2,6-dichlorophenyl)-1H-pyrazole-3-carboxamide; 4-((4-(4-chloro-3-methyl-1H-pyrazol-1-yl)phenyl)amino)-1-(2,6-dichlorophenyl)-1H-pyrazole-3-carboxamide; 1-(2,6-dichlorophenyl)-4-((4-(4-ethyl-3,5-dimethyl-1H-pyrazol-1-yl)phenyl)amino)-1H-pyrazole-3-carboxamide; 1-(2,6-dichlorophenyl)-4-((4-(3,5-diethyl-1H-pyrazol-1-yl)phenyl)amino)-1H-pyrazole-3-carboxamide; 1-(2,6-dichlorophenyl)-4-((4-(3,4,5-trimethyl-1H-pyrazol-1-yl)phenyl)amino)-1H-pyrazole-3-carboxamide; 1-(2,6-dichlorophenyl)-4-((4-(1-methyl-1H-imidazol-2-yl)phenyl)amino)-1H-pyrazole-3-carboxamide; 1-(2,6-dichlorophenyl)-4-((5-(pyrrolidine-1-carbonyl)pyridin-2-yl)amino)-1H-pyrazole-3-carboxamide; 1-(2-chloro-6-fluorophenyl)-4-((4-(pyrrolidine-1-carbonyl)phenyl)amino)-1H-pyrazole-3-carboxamide; 1-(2-chloro-6-fluorophenyl)-4-((4-(piperidine-1-carbonyl)phenyl)amino)-1H-pyrazole-3-carboxamide; 1-(2,6-dichlorophenyl)-4-((4-(1-ethyl-1H-imidazol-2-yl)phenyl)amino)-1H-pyrazole-3-carboxamide; 1-(2,6-dichlorophenyl)-4-((4-(1-(2,2-difluoroethyl)-1H-imidazol-2-yl)phenyl)amino)-1H-pyrazole-3-carboxamide; 1-(2-chloro-6-fluorophenyl)-4-((4-(1-methyl-3-(trifluoromethyl)-1H-1,2,4-triazol-5-yl)phenyl)amino)-1H-pyrazole-3-carboxamide; 1-(2-chloro-6-fluorophenyl)-4-((4-(3-methyl-1H-pyrazol-1-yl)phenyl)amino)-1H-pyrazole-3-carboxamide; 1-(2-chloro-6-fluorophenyl)-4-((4-(5-methyl-1H-pyrazol-1-yl)phenyl)amino)-1H-pyrazole-3-carboxamide; 1-(2,6-dichlorophenyl)-4-((4-(3-ethylpyridin-2-yl)phenyl)amino)-1H-pyrazole-3-carboxamide; 1-(2,6-dichlorophenyl)-4-((5-(pyrrolidine-1-carbonyl)pyrimidin-2-yl)amino)-1H-pyrazole-3-carboxamide; 1-(2,6-dichlorophenyl)-4-((4-(3-(trifluoromethyl)-4,5,6,7-tetrahydro-2H-indazol-2-yl)phenyl)amino)-1H-pyrazole-3-carboxamide; 1-(2,6-dichlorophenyl)-4-((4-((1-methylpiperidin-4-yl)oxy)phenyl)amino)-1H-pyrazole-3-carboxamide; 1-(2,6-dichlorophenyl)-4-((4-(1-methyl-1H-tetrazol-5-yl)phenyl)amino)-1H-pyrazole-3-carboxamide; 1-(2,6-dichlorophenyl)-4-((4′-fluoro-[1,1′-biphenyl]-4-yl)amino)-1H-pyrazole-3-carboxamide; 1-(2,6-dichlorophenyl)-4-((4-(3-ethyl-1H-pyrazol-1-yl)phenyl)amino)-1H-pyrazole-3-carboxamide; 1-(2,6-dichlorophenyl)-4-((4-(5-ethyl-1H-pyrazol-1-yl)phenyl)amino)-1H-pyrazole-3-carboxamide; 1-(2,6-dichlorophenyl)-4-((4-(4-methyl-5-(trifluoromethyl)-4H-1,2,4-triazol-3-yl)phenyl)amino)-1H-pyrazole-3-carboxamide; 1-(2,6-dichlorophenyl)-4-((4-(5-methyl-1H-tetrazol-1-yl)phenyl)amino)-1H-pyrazole-3-carboxamide; 1-(2,6-dichlorophenyl)-4-((3′-fluoro-[1,1′-biphenyl]-4-yl)amino)-1H-pyrazole-3-carboxamide; 1-(2,6-dichlorophenyl)-4-((2′,5′-difluoro-[1,1′-biphenyl]-4-yl)amino)-1H-pyrazole-3-carboxamide; 1-(2,6-dichlorophenyl)-4-((4-(3-fluoropyrrolidine-1-carbonyl)phenyl)amino)-1H-pyrazole-3-carboxamide; 1-(2,6-dichlorophenyl)-4-((4-(3,3-difluoropyrrolidine-1-carbonyl)phenyl)amino)-1H-pyrazole-3-carboxamide; 1-(2,6-dichlorophenyl)-4-((4-(3-(dimethylamino)pyrrolidine-1-carbonyl)phenyl)amino)-1H-pyrazole-3-carboxamide; 4-((4-([1,3′-bipyrrolidine]-1′-carbonyl)phenyl)amino)-1-(2,6-dichlorophenyl)-1H-pyrazole-3-carboxamide; 1-(2,6-dichlorophenyl)-4-((4-(5-ethyl-4-methyl-1H-pyrazol-1-yl)phenyl)amino)-1H-pyrazole-3-carboxamide; 1-(2,6-dichlorophenyl)-4-((4-(1-(2-(dimethylamino)ethyl)-1H-imidazol-2-yl)phenyl)amino)-1H-pyrazole-3-carboxamide; 4-((4-(4-cyano-3-methyl-1H-pyrazol-1-yl)phenyl)amino)-1-(2,6-dichlorophenyl)-1H-pyrazole-3-carboxamide; 4-((4-(4-cyano-5-methyl-1H-pyrazol-1-yl)phenyl)amino)-1-(2,6-dichlorophenyl)-1H-pyrazole-3-carboxamide; 1-(2,6-dichlorophenyl)-4-((4-(3,3,4,4-tetrafluoropyrrolidine-1-carbonyl)phenyl)amino)-1H-pyrazole-3-carboxamide; 1-(2,6-dichlorophenyl)-4-((2′-fluoro-[1,1′-biphenyl]-4-yl)amino)-1H-pyrazole-3-carboxamide; 4-((2′-cyano-[1,1′-biphenyl]-4-yl)amino)-1-(2,6-dichlorophenyl)-1H-pyrazole-3-carboxamide; 1-(2,6-dichlorophenyl)-4-((4-(5-methyl-3-(trifluoromethyl)-1H-pyrazol-1-yl)phenyl)amino)-1H-pyrazole-3-carboxamide; 1-(2,6-dichlorophenyl)-4-((4-(3-methyl-5-(trifluoromethyl)-1H-pyrazol-1-yl)phenyl)amino)-1H-pyrazole-3-carboxamide; 1-(2,6-dichlorophenyl)-4-((4-(5-ethyl-1H-tetrazol-1-yl)phenyl)amino)-1H-pyrazole-3-carboxamide; 1-(2,6-dichlorophenyl)-4-((4-(3-methylpyridin-4-yl)phenyl)amino)-1H-pyrazole-3-carboxamide; 1-(2,6-dichlorophenyl)-4-((4-isopropoxyphenyl)amino)-1H-pyrazole-3-carboxamide; 1-(2,6-dichlorophenyl)-4-((4-(5-isopropyl-1H-pyrazol-1-yl)phenyl)amino)-1H-pyrazole-3-carboxamide; 1-(2,6-dichlorophenyl)-4-((4-(3-isopropyl-1H-pyrazol-1-yl)phenyl)amino)-1H-pyrazole-3-carboxamide; 1-(2,6-dichlorophenyl)-N-methyl-4-((4-(morpholine-4-carbonyl)phenyl)amino)-1H-pyrazole-3-carboxamide; 1-(2,6-dichlorophenyl)-4-((4-sulfamoylphenyl)amino)-1H-pyrazole-3-carboxamide; 4-((4-cyanophenyl)amino)-1-(2,6-dichlorophenyl)-1H-pyrazole-3-carboxamide; 4-(4-((3-carbamoyl-1-(2,6-dichlorophenyl)-1H-pyrazol-4-yl)amino)phenyl)-3-methylpyridine_1-oxide; 1-(2,6-dichlorophenyl)-4-((4-(1-(2,2,2-trifluoroethyl)-1H-imidazol-2-yl)phenyl)amino)-1H-pyrazole-3-carboxamide; 1-(2,6-dichlorophenyl)-4-((4-(7-methyl-5,6,7,8-tetrahydro-[1,2,4]triazolo[4,3-a]pyrazin-3-yl)phenyl)amino)-1H-pyrazole-3-carboxamide; 1-(2,6-dichlorophenyl)-4-((4-(ethylsulfonamido)phenyl)amino)-1H-pyrazole-3-carboxamide; 1-(2,6-dichlorophenyl)-4-((4-(methylsulfonamido)phenyl)amino)-1H-pyrazole-3-carboxamide; 1-(2,6-dichlorophenyl)-4-((4-(1,1-dioxidoisothiazolidin-2-yl)phenyl)amino)-1H-pyrazole-3-carboxamide; 1-(2,6-dichlorophenyl)-4-((4-(5-(2-(dimethylamino)ethyl)-1H-tetrazol-1-yl)phenyl)amino)-1H-pyrazole-3-carboxamide; 1-(2,6-dichlorophenyl)-4-((4-(5-isopropyl-3-(trifluoromethyl)-1H-1,2,4-triazol-1-yl)phenyl)amino)-1H-pyrazole-3-carboxamide; 1-(2,6-dichlorophenyl)-4-((4-(5-methyl-3-(trifluoromethyl)-1H-1,2,4-triazol-1-yl)phenyl)amino)-1H-pyrazole-3-carboxamide; 4-((4-(3-cyanoazetidine-1-carbonyl)phenyl)amino)-1-(2,6-dichlorophenyl)-1H-pyrazole-3-carboxamide; 1-(2,6-dichlorophenyl)-4-((4-(5-ethyl-3-(trifluoromethyl)-1H-1,2,4-triazol-1-yl)phenyl)amino)-1H-pyrazole-3-carboxamide; 1-(2,6-dichlorophenyl)-4-((4-(1-ethyl-3-(trifluoromethyl)-1H-1,2,4-triazol-5-yl)phenyl)amino)-1H-pyrazole-3-carboxamide; 1-(2,6-dichlorophenyl)-4-((4-(5-propyl-3-(trifluoromethyl)-1H-1,2,4-triazol-1-yl)phenyl)amino)-1H-pyrazole-3-carboxamide; 1-(2,6-dichlorophenyl)-4-((4-(1-isopropyl-3-(trifluoromethyl)-1H-1,2,4-triazol-5-yl)phenyl)amino)-1H-pyrazole-3-carboxamide; 1-(2,6-dichlorophenyl)-4-((4-(5-(methoxymethyl)-3-(trifluoromethyl)-1H-1,2,4-triazol-1-yl)phenyl)amino)-1H-pyrazole-3-carboxamide; 1-(2,6-dichlorophenyl)-4-((4-(1-propyl-3-(trifluoromethyl)-1H-1,2,4-triazol-5-yl)phenyl)amino)-1H-pyrazole-3-carboxamide; 1-(2-chloro-6-fluorophenyl)-4-((4-(5-ethyl-3-(trifluoromethyl)-1H-1,2,4-triazol-1-yl)phenyl)amino)-1H-pyrazole-3-carboxamide; 1-(2,6-difluorophenyl)-4-((4-(5-ethyl-3-(trifluoromethyl)-1H-1,2,4-triazol-1-yl)phenyl)amino)-1H-pyrazole-3-carboxamide; 1-(2,6-difluorophenyl)-4-((4-(1-methyl-3-(trifluoromethyl)-1H-1,2,4-triazol-5-yl)phenyl)amino)-1H-pyrazole-3-carboxamide; 1-(2-chloro-6-fluorophenyl)-4-((4-(5-ethyl-1H-pyrazol-1-yl)phenyl)amino)-1H-pyrazole-3-carboxamide; 1-(2,6-difluorophenyl)-4-((4-(5-ethyl-1H-pyrazol-1-yl)phenyl)amino)-1H-pyrazole-3-carboxamide; 1-(2,6-difluorophenyl)-4-((4-((1-methylpiperidin-4-yl)oxy)phenyl)amino)-1H-pyrazole-3-carboxamide; 1-(2-chloro-6-fluorophenyl)-4-((4-(1-ethyl-3-(trifluoromethyl)-1H-1,2,4-triazol-5-yl)phenyl)amino)-1H-pyrazole-3-carboxamide; 4-((3-carbamoyl-1-(2,6-dichlorophenyl)-1H-pyrazol-4-yl)amino)benzenesulfonic_acid; 1-(2,6-dichlorophenyl)-4-((4-(5-ethyl-1H-1,2,4-triazol-1-yl)phenyl)amino)-1H-pyrazole-3-carboxamide; 1-(2,6-dichlorophenyl)-4-((4-(1-(methoxymethyl)-4-(trifluoromethyl)-1H-imidazol-2-yl)phenyl)amino)-1H-pyrazole-3-carboxamide; 1-(2,6-dichlorophenyl)-4-((4-(1-ethyl-4-(trifluoromethyl)-1H-imidazol-2-yl)phenyl)amino)-1H-pyrazole-3-carboxamide; 1-(2,6-dichlorophenyl)-4-((4-(1-methyl-4-(trifluoromethyl)-1H-imidazol-2-yl)phenyl)amino)-1H-pyrazole-3-carboxamide; 1-(2-chloro-6-fluorophenyl)-4-((4-((1-methylpiperidin-4-yl)oxy)phenyl)amino)-1H-pyrazole-3-carboxamide; 1-(2,6-difluorophenyl)-4-((4-(4-methylpyridazin-3-yl)phenyl)amino)-1H-pyrazole-3-carboxamide; 1-(2,6-dichlorophenyl)-4-((4-(5-((dimethylamino)methyl)-3-(trifluoromethyl)-1H-1,2,4-triazol-1-yl)phenyl)amino)-1H-pyrazole-3-carboxamide; 1-(2-chloro-6-fluorophenyl)-4-((4-(5-(methoxymethyl)-3-(trifluoromethyl)-1H-1,2,4-triazol-1-yl)phenyl)amino)-1H-pyrazole-3-carboxamide; 1-(2,6-difluorophenyl)-4-((4-(5-(methoxymethyl)-3-(trifluoromethyl)-1H-1,2,4-triazol-1-yl)phenyl)amino)-1H-pyrazole-3-carboxamide; 1-(2,6-dichlorophenyl)-4-((4-(5-(methoxymethyl)-1H-1,2,4-triazol-1-yl)phenyl)amino)-1H-pyrazole-3-carboxamide; 1-(2,6-dichlorophenyl)-4-((4-(1-(oxetan-3-yl)-4-(trifluoromethyl)-1H-imidazol-2-yl)phenyl)amino)-1H-pyrazole-3-carboxamide; 1-(2,6-difluorophenyl)-4-((4-(1-ethyl-3-(trifluoromethyl)-1H-1,2,4-triazol-5-yl)phenyl)amino)-1H-pyrazole-3-carboxamide; 1-(2-chloro-6-fluorophenyl)-4-((4-(1-ethyl-4-(trifluoromethyl)-1H-imidazol-2-yl)phenyl)amino)-1H-pyrazole-3-carboxamide; 1-(2,6-difluorophenyl)-4-((4-(1-ethyl-4-(trifluoromethyl)-1H-imidazol-2-yl)phenyl)amino)-1H-pyrazole-3-carboxamide; 1-(2-chloro-6-fluorophenyl)-4-((4-(1-methyl-4-(trifluoromethyl)-1H-imidazol-2-yl)phenyl)amino)-1H-pyrazole-3-carboxamide; 1-(2,6-dichlorophenyl)-4-((4-(1-(2-(dimethylamino)ethyl)-4-(trifluoromethyl)-1H-imidazol-2-yl)phenyl)amino)-1H-pyrazole-3-carboxamide; 1-(2,6-dichlorophenyl)-4-((4-(1-(2-methoxyethyl)-4-(trifluoromethyl)-1H-imidazol-2-yl)phenyl)amino)-1H-pyrazole-3-carboxamide; 1-(2,6-difluorophenyl)-4-((4-(1-methyl-4-(trifluoromethyl)-1H-imidazol-2-yl)phenyl)amino)-1H-pyrazole-3-carboxamide; 1-(2,6-difluorophenyl)-4-((4-(1-isopropyl-1H-1,2,4-triazol-5-yl)phenyl)amino)-1H-pyrazole-3-carboxamide; 4-((4-(4-cyano-1-ethyl-1H-imidazol-2-yl)phenyl)amino)-1-(2,6-dichlorophenyl)-1H-pyrazole-3-carboxamide; 1-(2,6-dichlorophenyl)-4-((4-(1-propyl-4-(trifluoromethyl)-1H-imidazol-2-yl)phenyl)amino)-1H-pyrazole-3-carboxamide; 4-((4-(1-butyl-4-(trifluoromethyl)-1H-imidazol-2-yl)phenyl)amino)-1-(2,6-dichlorophenyl)-1H-pyrazole-3-carboxamide; (R)-1-(2,6-dichlorophenyl)-4-((4-(3-(dimethylamino)pyrrolidine-1-carbonyl)phenyl)amino)-1H-pyrazole-3-carboxamide; (S)-1-(2,6-dichlorophenyl)-4-((4-(3-(dimethylamino)pyrrolidine-1-carbonyl)phenyl)amino)-1H-pyrazole-3-carboxamide; (R)-1-(2,6-dichlorophenyl)-4-((4-(3-fluoropyrrolidine-1-carbonyl)phenyl)amino)-1H-pyrazole-3-carboxamide; (S)-1-(2,6-dichlorophenyl)-4-((4-(3-fluoropyrrolidine-1-carbonyl)phenyl)amino)-1H-pyrazole-3-carboxamide; 4-((4-(3-aminopyrrolidine-1-carbonyl)phenyl)amino)-1-(2,6-dichlorophenyl)-1H-pyrazole-3-carboxamide; (R)-4-((4-(3-aminopyrrolidine-1-carbonyl)phenyl)amino)-1-(2,6-dichlorophenyl)-1H-pyrazole-3-carboxamide; (S)-4-((4-(3-aminopyrrolidine-1-carbonyl)phenyl)amino)-1-(2,6-dichlorophenyl)-1H-pyrazole-3-carboxamide; 1-(2,6-dichlorophenyl)-4-((2-(2-methyl-1H-imidazol-1-yl)thiazol-4-yl)amino)-1H-pyrazole-3-carboxamide; 1-(2,6-dichlorophenyl)-4-((4-(pyridazin-4-yl)phenyl)amino)-1H-pyrazole-3-carboxamide; 1-(2,6-dichlorophenyl)-4-((4-(5-ethylpyridazin-4-yl)phenyl)amino)-1H-pyrazole-3-carboxamide; tert-butyl_(1-(4-((3-carbamoyl-1-(2,6-dichlorophenyl)-1H-pyrazol-4-yl)amino)benzoyl)pyrrolidin-3-yl)carbamate; (R)-tert-butyl_(1-(4-((3-carbamoyl-1-(2,6-dichlorophenyl)-1H-pyrazol-4-yl)amino)benzoyl)pyrrolidin-3-yl)carbamate; (S)-tert-butyl_(1-(4-((3-carbamoyl-1-(2,6-dichlorophenyl)-1H-pyrazol-4-yl)amino)benzoyl)pyrrolidin-3-yl)carbamate; 1-(2,6-dichlorophenyl)-4-((4-(5-methyl-1H-1,2,4-triazol-1-yl)phenyl)amino)-1H-pyrazole-3-carboxamide; 1-(2,6-dichlorophenyl)-4-((4-(5-(2-methoxyethyl)-1H-1,2,4-triazol-1-yl)phenyl)amino)-1H-pyrazole-3-carboxamide; 1-(2,6-dichlorophenyl)-4-((6-(5-(methoxymethyl)-3-(trifluoromethyl)-1H-1,2,4-triazol-1-yl)pyridin-3-yl)amino)-1H-pyrazole-3-carboxamide; 1-(2,6-dichlorophenyl)-4-((6-(5-ethyl-3-(trifluoromethyl)-1H-1,2,4-triazol-1-yl)pyridin-3-yl)amino)-1H-pyrazole-3-carboxamide; 1-(2,6-dichlorophenyl)-4-((4-(5-(2-methoxyethyl)-3-(trifluoromethyl)-1H-1,2,4-triazol-1-yl)phenyl)amino)-1H-pyrazole-3-carboxamide; (R)-4-((4-([1,3′-bipyrrolidine]-1′-carbonyl)phenyl)amino)-1-(2,6-dichlorophenyl)-1H-pyrazole-3-carboxamide; (S)-4-((4-([1,3′-bipyrrolidine]-1′-carbonyl)phenyl)amino)-1-(2,6-dichlorophenyl)-1H-pyrazole-3-carboxamide; 4-((4-([1,2,4]triazolo[4,3-a]pyridin-3-yl)phenyl)amino)-1-(2,6-dichlorophenyl)-1H-pyrazole-3-carboxamide; 1-(2,6-dichlorophenyl)-4-((4-(5-(trifluoromethyl)-1H-1,2,4-triazol-3-yl)phenyl)amino)-1H-pyrazole-3-carboxamide; 1-(2,6-dichlorophenyl)-4-((6-(5-(methoxymethyl)-3-(trifluoromethyl)-1H-1,2,4-triazol-1-yl)pyridazin-3-yl)amino)-1H-pyrazole-3-carboxamide; 1-(2,6-dichlorophenyl)-4-((6-(5-ethyl-3-(trifluoromethyl)-1H-1,2,4-triazol-1-yl)pyridazin-3-yl)amino)-1H-pyrazole-3-carboxamide; 4-((6-(1H-1,2,4-triazol-1-yl)pyridin-3-yl)amino)-1-(2,6-dichlorophenyl)-1H-pyrazole-3-carboxamide; 1-(2,6-dichlorophenyl)-4-((4-(1-(2-hydroxyethyl)-4-(trifluoromethyl)-1H-imidazol-2-yl)phenyl)amino)-1H-pyrazole-3-carboxamide; 1-(2,6-dichlorophenyl)-4-((6-(1-ethyl-4-(trifluoromethyl)-1H-imidazol-2-yl)pyridin-3-yl)amino)-1H-pyrazole-3-carboxamide; 1-(2,6-dichlorophenyl)-4-((4-(5-methylpyridazin-4-yl)phenyl)amino)-1H-pyrazole-3-carboxamide; 1-(2,6-dichlorophenyl)-4-((4-(5-isopropyl-1H-1,2,4-triazol-1-yl)phenyl)amino)-1H-pyrazole-3-carboxamide; 4-((4-(1-(1-acetylazetidin-3-yl)-4-(trifluoromethyl)-1H-imidazol-2-yl)phenyl)amino)-1-(2,6-dichlorophenyl)-1H-pyrazole-3-carboxamide; 1-(2,6-dichlorophenyl)-4-((6-(5-ethyl-1H-1,2,4-triazol-1-yl)pyridin-3-yl)amino)-1H-pyrazole-3-carboxamide; 1-(2,6-dichlorophenyl)-4-((6-(2-methoxyphenyl)pyridin-3-yl)amino)-1H-pyrazole-3-carboxamide; 1-(2,6-dichlorophenyl)-4-((6-(2-oxopyrrolidin-1-yl)pyridin-3-yl)amino)-1H-pyrazole-3-carboxamide; 1-(2,6-dichlorophenyl)-4-((5-methyl-6-(1H-pyrazol-1-yl)pyridin-3-yl)amino)-1H-pyrazole-3-carboxamide; 1-(2,6-dichlorophenyl)-4-((4-(5-(oxetan-3-yl)-1H-1,2,4-triazol-1-yl)phenyl)amino)-1H-pyrazole-3-carboxamide; 1-(2,6-dichlorophenyl)-4-((4-(5-(hydroxymethyl)-1H-1,2,4-triazol-1-yl)phenyl)amino)-1H-pyrazole-3-carboxamide; 4-((4-(4-cyano-1-(oxetan-3-yl)-1H-imidazol-2-yl)phenyl)amino)-1-(2,6-dichlorophenyl)-1H-pyrazole-3-carboxamide; 1-(2,6-dichlorophenyl)-4-((6-(5-methylpyridazin-4-yl)pyridin-3-yl)amino)-1H-pyrazole-3-carboxamide; 1-(2,6-dichlorophenyl)-4-((6-(5-ethylpyridazin-4-yl)pyridin-3-yl)amino)-1H-pyrazole-3-carboxamide; 1-(2,6-dichlorophenyl)-4-((6-(pyridazin-4-yl)pyridin-3-yl)amino)-1H-pyrazole-3-carboxamide; 1-(2,6-dichlorophenyl)-4-((6-oxo-1,6-dihydropyridin-3-yl)amino)-1H-pyrazole-3-carboxamide; (S)-1-(2,6-dichlorophenyl)-4-((6-(3-(dimethylamino)pyrrolidine-1-carbonyl)pyridin-3-yl)amino)-1H-pyrazole-3-carboxamide; (S)-1-(2,6-dichlorophenyl)-4-((6-(3-fluoropyrrolidine-1-carbonyl)pyridin-3-yl)amino)-1H-pyrazole-3-carboxamide; (R)-4-((6-([1,3′-bipyrrolidine]-1′-carbonyl)pyridin-3-yl)amino)-1-(2,6-dichlorophenyl)-1H-pyrazole-3-carboxamide; (S)-4-((6-(3-aminopyrrolidine-1-carbonyl)pyridin-3-yl)amino)-1-(2,6-dichlorophenyl)-1H-pyrazole-3-carboxamide; 1-(2,6-dichlorophenyl)-4-((6-(pyrrolidine-1-carbonyl)pyridin-3-yl)amino)-1H-pyrazole-3-carboxamide; 1-(2-chloro-6-fluorophenyl)-4-((1-methyl-1H-pyrazol-4-yl)amino)-1H-pyrazole-3-carboxamide; 1-(2,6-dichlorophenyl)-4-((6-(4-methyl-4H-1,2,4-triazol-3-yl)pyridin-3-yl)amino)-1H-pyrazole-3-carboxamide; 4-((4-(4-cyano-1-(2-hydroxyethyl)-1H-imidazol-2-yl)phenyl)amino)-1-(2,6-dichlorophenyl)-1H-pyrazole-3-carboxamide; 1-(2,6-dichlorophenyl)-4-((6-(3,3,4,4-tetrafluoropyrrolidine-1-carbonyl)pyridin-3-yl)amino)-1H-pyrazole-3-carboxamide; 1-(2,6-dichlorophenyl)-4-((6-(4-methylpiperazine-1-carbonyl)pyridin-3-yl)amino)-1H-pyrazole-3-carboxamide; 4-((6-(4-cyclopropylpiperazine-1-carbonyl)pyridin-3-yl)amino)-1-(2,6-dichlorophenyl)-1H-pyrazole-3-carboxamide; 1-(2,6-dichlorophenyl)-4-((6-(morpholine-4-carbonyl)pyridin-3-yl)amino)-1H-pyrazole-3-carboxamide; (S)-1-(2,6-dichlorophenyl)-4-((5-(3-(dimethylamino)pyrrolidine-1-carbonyl)pyridin-2-yl)amino)-1H-pyrazole-3-carboxamide; (S)-1-(2,6-dichlorophenyl)-4-((5-(3-fluoropyrrolidine-1-carbonyl)pyridin-2-yl)amino)-1H-pyrazole-3-carboxamide; (S)-4-((5-(3-aminopyrrolidine-1-carbonyl)pyridin-2-yl)amino)-1-(2,6-dichlorophenyl)-1H-pyrazole-3-carboxamide; 1-(2,6-dichlorophenyl)-4-((5-(3,3,4,4-tetrafluoropyrrolidine-1-carbonyl)pyridin-2-yl)amino)-1H-pyrazole-3-carboxamide; 1-(2,6-dichlorophenyl)-4-((5-(4-methylpiperazine-1-carbonyl)pyridin-2-yl)amino)-1H-pyrazole-3-carboxamide; 1-(2,6-dichlorophenyl)-4-((4-(5-(hydroxymethyl)-3-(trifluoromethyl)-1H-1,2,4-triazol-1-yl)phenyl)amino)-1H-pyrazole-3-carboxamide; 1-(2-chloro-6-ethoxyphenyl)-4-((1-methyl-1H-pyrazol-4-yl)amino)-1H-pyrazole-3-carboxamide; 1-(2,6-dichlorophenyl)-4-((6-(1-(2-hydroxyethyl)-4-(trifluoromethyl)-1H-imidazol-2-yl)pyridin-3-yl)amino)-1H-pyrazole-3-carboxamide; 1-(2,6-dichlorophenyl)-4-((1-(3,6-dimethylpyrazin-2-yl)-1H-pyrazol-4-yl)amino)-1H-pyrazole-3-carboxamide; 1-(2,6-dichlorophenyl)-4-((6-(1-methyl-1H-tetrazol-5-yl)pyridin-3-yl)amino)-1H-pyrazole-3-carboxamide; 1-(2,6-dichlorophenyl)-4-((4-(5-methyl-[1,2,4]triazolo[4,3-a]pyridin-3-yl)phenyl)amino)-1H-pyrazole-3-carboxamide; 1-(2,6-dichlorophenyl)-4-((1-methyl-1H-pyrazol-4-yl)amino)-1H-pyrazole-3-carboxamide; 1-(2,6-dichlorophenyl)-4-((1-isopropyl-1H-pyrazol-4-yl)amino)-1H-pyrazole-3-carboxamide; 1-(2,6-dichlorophenyl)-4-((2-methyl-2H-1,2,3-triazol-4-yl)amino)-1H-pyrazole-3-carboxamide; 1-(2,6-dichlorophenyl)-4-((1-methyl-1H-1,2,3-triazol-4-yl)amino)-1H-pyrazole-3-carboxamide; 1-(2,6-dichlorophenyl)-4-((1-methyl-2-oxo-1,2-dihydropyridin-4-yl)amino)-1H-pyrazole-3-carboxamide; (R)-4-((5-([1,3′-bipyrrolidine]-1′-carbonyl)pyridin-2-yl)amino)-1-(2,6-dichlorophenyl)-1H-pyrazole-3-carboxamide; 4-((5-(4-cyclopropylpiperazine-1-carbonyl)pyridin-2-yl)amino)-1-(2,6-dichlorophenyl)-1H-pyrazole-3-carboxamide; 1-(2,6-dichlorophenyl)-4-((6-(2-oxopiperidin-1-yl)pyridin-3-yl)amino)-1H-pyrazole-3-carboxamide; 1-(2,6-dichlorophenyl)-4-((6-(5-(hydroxymethyl)-3-(trifluoromethyl)-1H-1,2,4-triazol-1-yl)pyridin-3-yl)amino)-1H-pyrazole-3-carboxamide; 1-(2,6-dichlorophenyl)-4-((1-(3-fluoropyridin-2-yl)-1H-pyrazol-4-yl)amino)-1H-pyrazole-3-carboxamide; 4-((6-(4-cyano-1-(2-hydroxyethyl)-1H-imidazol-2-yl)pyridin-3-yl)amino)-1-(2,6-dichlorophenyl)-1H-pyrazole-3-carboxamide; 1-(2,6-dichlorophenyl)-4-((6-(5-methyl-[1,2,4]triazolo[4,3-a]pyridin-3-yl)pyridin-3-yl)amino)-1H-pyrazole-3-carboxamide; 1-(2,6-dichlorophenyl)-4-((1-methyl-6-oxo-1,6-dihydropyridin-3-yl)amino)-1H-pyrazole-3-carboxamide; 1-(2,6-dichlorophenyl)-4-((2-oxo-2H-[1,2′-bipyridin]-5′-yl)amino)-1H-pyrazole-3-carboxamide; 1-(2,6-dichlorophenyl)-4-((4-(5-methyl-1H-1,2,3-triazol-1-yl)phenyl)amino)-1H-pyrazole-3-carboxamide; 1-(2,6-dichlorophenyl)-4-((1-(4-methoxybenzyl)-1H-pyrazol-4-yl)amino)-1H-pyrazole-3-carboxamide; 1-(2,6-dichlorophenyl)-4-((6-(5-isopropyl-1H-tetrazol-1-yl)pyridin-3-yl)amino)-1H-pyrazole-3-carboxamide; 1-(2,6-dichlorophenyl)-4-((4′-methyl-[2,3′-bipyridin]-5-yl)amino)-1H-pyrazole-3-carboxamide; 1-(2,6-dichlorophenyl)-4-((3′-methyl-[2,2′-bipyridin]-5-yl)amino)-1H-pyrazole-3-carboxamide; 4-((1-(3-chloropyridin-2-yl)-1H-pyrazol-3-yl)amino)-1-(2,6-dichlorophenyl)-1H-pyrazole-3-carboxamide; 1-(2,6-dichlorophenyl)-4-((1-(pyridin-2-yl)-1H-pyrazol-3-yl)amino)-1H-pyrazole-3-carboxamide; 1-(2,6-dichlorophenyl)-4-((6-(3,5-dimethyl-1H-pyrazol-1-yl)pyridin-3-yl)amino)-1H-pyrazole-3-carboxamide; 1-(2,6-dichlorophenyl)-4-((1-(pyridin-2-yl)-1H-imidazol-4-yl)amino)-1H-pyrazole-3-carboxamide; 1-(2,6-dichlorophenyl)-4-((6-(4-isopropyl-4H-1,2,4-triazol-3-yl)pyridin-3-yl)amino)-1H-pyrazole-3-carboxamide; 1-(2,6-dichlorophenyl)-4-((5-(morpholine-4-carbonyl)pyrazin-2-yl)amino)-1H-pyrazole-3-carboxamide; 1-(2,6-dichlorophenyl)-4-((5-(4-methylpiperazine-1-carbonyl)pyrazin-2-yl)amino)-1H-pyrazole-3-carboxamide; (R)-1-(2,6-dichlorophenyl)-4-((5-(3-fluoropyrrolidine-1-carbonyl)pyrazin-2-yl)amino)-1H-pyrazole-3-carboxamide; 1-(2-chloro-6-fluorophenyl)-4-((1-methyl-1H-1,2,4-triazol-5-yl)amino)-1H-pyrazole-3-carboxamide; 1-(2,6-dichlorophenyl)-4-((6-((1-methylpiperidin-4-yl)oxy)pyridin-3-yl)amino)-1H-pyrazole-3-carboxamide; 1-(2,6-dichlorophenyl)-4-((1-methyl-1H-1,2,4-triazol-5-yl)amino)-1H-pyrazole-3-carboxamide; 4-((1H-pyrazol-4-yl)amino)-1-(2,6-dichlorophenyl)-1H-pyrazole-3-carboxamide; 1-(2,6-dichlorophenyl)-4-(imidazo[1,2-b]pyridazin-3-ylamino)-1H-pyrazole-3-carboxamide; 1-(2,6-dichlorophenyl)-4-(pyrazolo[1,5-a]pyrimidin-3-ylamino)-1H-pyrazole-3-carboxamide; 1-(2,6-dichlorophenyl)-4-((2-((1-methylpiperidin-4-yl)amino)pyrimidin-5-yl)amino)-1H-pyrazole-3-carboxamide; 1-(2,6-dichlorophenyl)-4-((4-(5-ethyl-1H-1,2,3-triazol-1-yl)phenyl)amino)-1H-pyrazole-3-carboxamide; 1-(2,6-dichloro-4-fluorophenyl)-4-((4-(morpholine-4-carbonyl)phenyl)amino)-1H-pyrazole-3-carboxamide; 1-(2,6-dichloro-4-fluorophenyl)-4-((4-(methyl(1-methylpiperidin-4-yl)carbamoyl)phenyl)amino)-1H-pyrazole-3-carboxamide; 1-(2,6-dichloro-4-fluorophenyl)-4-((4-(4-ethyl-4H-1,2,4-triazol-3-yl)phenyl)amino)-1H-pyrazole-3-carboxamide; (E)-1-(2,6-dichlorophenyl)-4-((4-(4-(2-fluorovinyl)-4H-1,2,4-triazol-3-yl)phenyl)amino)-1H-pyrazole-3-carboxamide; 1-(2,6-dichloro-3-fluorophenyl)-4-((4-(morpholine-4-carbonyl)phenyl)amino)-1H-pyrazole-3-carboxamide; 1-(2,6-dichloro-3-fluorophenyl)-4-((4-(methyl(1-methylpiperidin-4-yl)carbamoyl)phenyl)amino)-1H-pyrazole-3-carboxamide; 1-(2,6-dichloro-3-fluorophenyl)-4-((4-(4-ethyl-4H-1,2,4-triazol-3-yl)phenyl)amino)-1H-pyrazole-3-carboxamide; 1-(2,6-dichlorophenyl)-4-((4-((2-methoxyethyl)carbamoyl)phenyl)amino)-1H-pyrazole-3-carboxamide; 1-(2,6-dichlorophenyl)-4-((4-((3-methoxypropyl)carbamoyl)phenyl)amino)-1H-pyrazole-3-carboxamide; 1-(2,6-dichlorophenyl)-4-((4-((2-fluoroethyl)carbamoyl)phenyl)amino)-1H-pyrazole-3-carboxamide; 1-(2,6-dichlorophenyl)-4-((4-((2-(diethylamino)ethyl)carbamoyl)phenyl)amino)-1H-pyrazole-3-carboxamide; 1-(2,6-dichlorophenyl)-4-((4-(4-(dimethylamino)piperidine-1-carbonyl)phenyl)amino)-1H-pyrazole-3-carboxamide; 1-(2,6-dichlorophenyl)-4-((4-(piperidin-4-yloxy)phenyl)amino)-1H-pyrazole-3-carboxamide; 1-(2,6-dichlorophenyl)-4-((4-(3-fluoroazetidine-1-carbonyl)phenyl)amino)-1H-pyrazole-3-carboxamide; 1-(2,6-dichlorophenyl)-4-((4-(3-hydroxyazetidine-1-carbonyl)phenyl)amino)-1H-pyrazole-3-carboxamide; 1-(2,6-dichlorophenyl)-4-((6-(4-methyl-3H-indazol-3-ylidene)-1,6-dihydropyridin-3-yl)amino)-1H-pyrazole-3-carboxamide; 1-(2,6-dichlorophenyl)-4-((5-morpholinopyridin-2-yl)amino)-1H-pyrazole-3-carboxamide; 1-(2,6-dichlorophenyl)-4-((5-(5-methyl-[1,2,4]triazolo[4,3-a]pyridin-3-yl)pyridin-2-yl)amino)-1H-pyrazole-3-carboxamide; 1-(2,6-dichlorophenyl)-4-((5-(5-methyl-1H-1,2,4-triazol-1-yl)pyridin-2-yl)amino)-1H-pyrazole-3-carboxamide; 1-(2,6-dichlorophenyl)-4-((5-(1-ethyl-1H-1,2,4-triazol-5-yl)pyridin-2-yl)amino)-1H-pyrazole-3-carboxamide; 1-(2,6-dichlorophenyl)-4-((5-(5-methyl-1H-1,2,3-triazol-1-yl)pyridin-2-yl)amino)-1H-pyrazole-3-carboxamide; 1-(2,6-dichlorophenyl)-4-((5-(1-methyl-4-(trifluoromethyl)-1H-imidazol-2-yl)pyridin-2-yl)amino)-1H-pyrazole-3-carboxamide; 1-(2,6-dichlorophenyl)-4-((5-(2-methyl-2H-tetrazol-5-yl)pyridin-2-yl)amino)-1H-pyrazole-3-carboxamide; 1-(2,6-dichlorophenyl)-4-((5-(5-methyl-1H-tetrazol-1-yl)pyridin-2-yl)amino)-1H-pyrazole-3-carboxamide; 1-(2,6-dichlorophenyl)-4-((6-(5-methyl-[1,2,4]triazolo[4,3-a]pyrazin-3-yl)pyridin-3-yl)amino)-1H-pyrazole-3-carboxamide; 1-(2,6-dichlorophenyl)-4-((5-(5-(trifluoromethyl)-1H-1,2,3-triazol-1-yl)pyridin-2-yl)amino)-1H-pyrazole-3-carboxamide; 1-(2,6-dichlorophenyl)-4-((4-(5-(trifluoromethyl)-1H-1,2,3-triazol-1-yl)phenyl)amino)-1H-pyrazole-3-carboxamide; 1-(2,6-dichlorophenyl)-4-((5-(3-hydroxyazetidine-1-carbonyl)pyridin-2-yl)amino)-1H-pyrazole-3-carboxamide; 1-(2,6-dichlorophenyl)-4-((6-(2-morpholino-2-oxoethyl)pyridin-3-yl)amino)-1H-pyrazole-3-carboxamide; 1-(2,6-dichlorophenyl)-4-((6-(2-(3-fluoroazetidin-1-yl)-2-oxoethyl)pyridin-3-yl)amino)-1H-pyrazole-3-carboxamide; 1-(2,6-dichlorophenyl)-4-((5-(4-(dimethylamino)piperidine-1-carbonyl)pyridin-2-yl)amino)-1H-pyrazole-3-carboxamide; (R)-1-(2,6-dichlorophenyl)-4-((5-(3-fluoropyrrolidine-1-carbonyl)pyridin-2-yl)amino)-1H-pyrazole-3-carboxamide; 4-((5-(3-cyanoazetidine-1-carbonyl)pyridin-2-yl)amino)-1-(2,6-dichlorophenyl)-1H-pyrazole-3-carboxamide; 1-(2,6-dichlorophenyl)-4-((5-(1-methyl-1H-tetrazol-5-yl)pyridin-2-yl)amino)-1H-pyrazole-3-carboxamide; 1-(2,6-dichlorophenyl)-4-((6-(5-(trifluoromethyl)-1H-1,2,3-triazol-1-yl)pyridin-3-yl)amino)-1H-pyrazole-3-carboxamide; 4-((6-(5-chloro-[1,2,4]triazolo[4,3-a]pyrazin-3-yl)pyridin-3-yl)amino)-1-(2,6-dichlorophenyl)-1H-pyrazole-3-carboxamide; 1-(2,6-dichlorophenyl)-4-((5-(4-methyl-1,4-diazepane-1-carbonyl)pyridin-2-yl)amino)-1H-pyrazole-3-carboxamide; 1-(2,6-dichlorophenyl)-4-((5-(3-fluoroazetidine-1-carbonyl)pyridin-2-yl)amino)-1H-pyrazole-3-carboxamide; (R)-1-(2-cyano-6-fluorophenyl)-4-((4-(3-fluoropyrrolidine-1-carbonyl)phenyl)amino)-1H-pyrazole-3-carboxamide; 1-(2,6-dichlorophenyl)-4-((4-(1,1-dioxidothiomorpholine-4-carbonyl)phenyl)amino)-1H-pyrazole-3-carboxamide; 1-(2-cyano-6-fluorophenyl)-4-((4-(1,1-dioxidothiomorpholine-4-carbonyl)phenyl)amino)-1H-pyrazole-3-carboxamide; 4-[4-(3-Carbamoyl-azetidine-1-carbonyl)-phenylamino]-1-(2,6-dichloro-phenyl)-1H-pyrazole-3-carboxylic acid amide; 1-(2-cyano-6-fluorophenyl)-4-((4-(morpholine-4-carbonyl)phenyl)amino)-1H-pyrazole-3-carboxamide; 1-(2,6-dichlorophenyl)-4-((6-((1,1-dioxidothiomorpholino)methyl)pyridin-3-yl)amino)-1H-pyrazole-3-carboxamide; 1-(2,6-dichlorophenyl)-4-((4-(3,3-dimethylazetidine-1-carbonyl)phenyl)amino)-1H-pyrazole-3-carboxamide; 1-(2-chloro-6-cyanophenyl)-4-((4-(1,1-dioxidothiomorpholine-4-carbonyl)phenyl)amino)-1H-pyrazole-3-carboxamide; 1-(2-chloro-6-cyanophenyl)-4-((4-(morpholine-4-carbonyl)phenyl)amino)-1H-pyrazole-3-carboxamide; 1-(2-Chloro-6-cyano-phenyl)-4-[4-((R)-3-fluoro-pyrrolidine-1-carbonyl)-phenylamino]-1H-pyrazole-3-carboxylic acid amide; 1-(2,6-dichlorophenyl)-4-((6-(5-(1,1-difluoroethyl)-1H-tetrazol-1-yl)pyridin-3-yl)amino)-1H-pyrazole-3-carboxamide; 1-(2,6-dichlorophenyl)-4-((1-(2-morpholino-2-oxoethyl)-1H-pyrazol-4-yl)amino)-1H-pyrazole-3-carboxamide; methyl_2-(4-((3-carbamoyl-1-(2,6-dichlorophenyl)-1H-pyrazol-4-yl)amino)-1H-pyrazol-1-yl)acetate; 1-(2,6-dichlorophenyl)-4-((6-(1,1-dioxidothiomorpholine-4-carbonyl)pyridin-3-yl)amino)-1H-pyrazole-3-carboxamide; 1-(2,6-dichlorophenyl)-4-((6-(3,3-dimethylazetidine-1-carbonyl)pyridin-3-yl)amino)-1H-pyrazole-3-carboxamide; 1-(2,6-dichlorophenyl)-4-((5-(3,3-dimethylazetidine-1-carbonyl)pyridin-2-yl)amino)-1H-pyrazole-3-carboxamide; 1-(2,6-dichlorophenyl)-4-((4-(1-(methylimino)-1-oxidothiomorpholine-4-carbonyl)phenyl)amino)-1H-pyrazole-3-carboxamide; 1-(2,6-dichlorophenyl)-4-((4-(thiomorpholine-4-carbonyl)phenyl)amino)-1H-pyrazole-3-carboxamide; 1-(2,6-dichlorophenyl)-4-((4-(1-imino-1-oxidothiomorpholine-4-carbonyl)phenyl)amino)-1H-pyrazole-3-carboxamide; 1-(2,6-dichlorophenyl)-4-((1-(2-(1,1-dioxidothiomorpholino)-2-oxoethyl)-1H-pyrazol-4-yl)amino)-1H-pyrazole-3-carboxamide; 1-(2,6-dichlorophenyl)-4-((6-(5,7-dimethyl-[1,2,4]triazolo[4,3-c]pyrimidin-3-yl)pyridin-3-yl)amino)-1H-pyrazole-3-carboxamide; 1-(2,6-dichlorophenyl)-4-((6-(5-(trifluoromethyl)-[1,2,4]triazolo[4,3-a]pyridin-3-yl)pyridin-3-yl)amino)-1H-pyrazole-3-carboxamide; 1-(2,6-dichlorophenyl)-4-((6-(5-(trifluoromethyl)-[1,2,4]triazolo[4,3-a]pyrazin-3-yl)pyridin-3-yl)amino)-1H-pyrazole-3-carboxamide; 1-(2,6-dichlorophenyl)-4-((6-(5-methylimidazo[1,2-a]pyridin-3-yl)pyridin-3-yl)amino)-1H-pyrazole-3-carboxamide; 1-(2,6-dichlorophenyl)-4-((6-(5-methyl-[1,2,4]triazolo[4,3-c]pyrimidin-3-yl)pyridin-3-yl)amino)-1H-pyrazole-3-carboxamide; 1-(2,6-dichlorophenyl)-4-((6-(5-methylthiazolo[2,3-c][1,2,4]triazol-3-yl)pyridin-3-yl)amino)-1H-pyrazole-3-carboxamide; 4-((6-(5-cyano-[1,2,4]triazolo[4,3-a]pyridin-3-yl)pyridin-3-yl)amino)-1-(2,6-dichlorophenyl)-1H-pyrazole-3-carboxamide; 1-(2,6-dichloro-4-fluorophenyl)-4-((6-(5-methyl-[1,2,4]triazolo[4,3-a]pyridin-3-yl)pyridin-3-yl)amino)-1H-pyrazole-3-carboxamide; and 4-((4-(3-carbamoylazetidine-1-carbonyl)phenyl)amino)-1-(2,6-dichlorophenyl)-1H-pyrazole-3-carboxamide.

40. The method of claim 21, wherein the compound is selected from the group consisting of 2-(2,6-Dichloro-phenyl)-5-[4-(morpholine-4-carbonyl)-phenylamino]-2H-[1,2,3]triazole-4-carboxylic acid amide; 2-(2,6-Dichloro-phenyl)-5-[4-(4-methyl-piperazine-1-carbonyl)-phenylamino]-2H-[1,2,3]triazole-4-carboxylic acid amide; 2-(2,6-Dichloro-phenyl)-5-[4-(1H-tetrazol-5-yl)-phenylamino]-2H-[1,2,3]triazole-4-carboxylic acid amide; 2-(2,6-Dichloro-phenyl)-5-[4-(1-methyl-1H-tetrazol-5-yl)-phenylamino]-2H-[1,2,3]triazole-4-carboxylic acid amide; 2-(2,6-Dichloro-phenyl)-5-[4-((S)-3-dimethylamino-pyrrolidine-1-carbonyl)-phenylamino]-2H-[1,2,3]triazole-4-carboxylic acid amide; 2-(2,6-Dichloro-phenyl)-5-[4-((R)-3-fluoro-pyrrolidine-1-carbonyl)-phenylamino]-2H-[1,2,3]triazole-4-carboxylic acid amide; 5-[4-(3-Cyano-azetidine-1-carbonyl)-phenylamino]-2-(2,6-dichloro-phenyl)-2H-[1,2,3]triazole-4-carboxylic acid amide; 2-(2,6-dichlorophenyl)-5-((4-(1-(2-hydroxyethyl)-4-(trifluoromethyl)-1H-imidazol-2-yl)phenyl)amino)-2H-1,2,3-triazole-4-carboxamide; 2-(2,6-Dichloro-phenyl)-5-[4-(5,6,7,8-tetrahydro-[1,2,4]triazolo[4,3-a]pyridin-3-yl)-phenylamino]-2H-[1,2,3]triazole-4-carboxylic acid amide; 2-(2,6-Dichloro-phenyl)-5-[4-(6,7-dihydro-4H-pyrano[4,3-c]pyrazol-1-yl)-phenylamino]-2H-[1,2,3]triazole-4-carboxylic acid amide; 2-(2,6-Dichloro-phenyl)-5-[4-(6,7-dihydro-4H-pyrano[4,3-c]pyrazol-2-yl)-phenylamino]-2H-[1,2,3]triazole-4-carboxylic acid amide; 2-(2,6-dichlorophenyl)-5-((4-(1-(2-hydroxyethyl)-4-(trifluoromethyl)-1H-imidazol-2-yl)phenyl)amino)-2H-1,2,3-triazole-4-carboxamide; 2-(2,6-dichlorophenyl)-5-((4-(4-(2,2-difluoroethyl)-4H-1,2,4-triazol-3-yl)phenyl)amino)-2H-1,2,3-triazole-4-carboxamide; 2-(2,6-dichlorophenyl)-5-((4-((1-methylpiperidin-4-yl)oxy)phenyl)amino)-2H-1,2,3-triazole-4-carboxamide; 2-(2,6-dichlorophenyl)-5-((4-(1-methyl-4-(trifluoromethyl)-1H-imidazol-2-yl)phenyl)amino)-2H-1,2,3-triazole-4-carboxamide; 2-(2,6-dichlorophenyl)-5-((4-(4-isopropyl-4H-1,2,4-triazol-3-yl)phenyl)amino)-2H-1,2,3-triazole-4-carboxamide; 2-(2,6-dichlorophenyl)-5-((4-(4-methyl-4H-1,2,4-triazol-3-yl)phenyl)amino)-2H-1,2,3-triazole-4-carboxamide; 2-(2,6-dichlorophenyl)-5-((4-(1-(2-methoxyethyl)-4-(trifluoromethyl)-1H-imidazol-2-yl)phenyl)amino)-2H-1,2,3-triazole-4-carboxamide; 2-(2,6-dichlorophenyl)-5-((4-((3-methoxypropyl)carbamoyl)phenyl)amino)-2H-1,2,3-triazole-4-carboxamide; 2-(2,6-dichlorophenyl)-5-((4-(piperidin-4-yloxy)phenyl)amino)-2H-1,2,3-triazole-4-carboxamide; 2-(2,6-dichlorophenyl)-5-((4-(5-ethyl-3-(trifluoromethyl)-1H-1,2,4-triazol-1-yl)phenyl)amino)-2H-1,2,3-triazole-4-carboxamide; 2-(2,6-dichlorophenyl)-5-((4-(4-(dimethylamino)piperidine-1-carbonyl)phenyl)amino)-2H-1,2,3-triazole-4-carboxamide; 2-(2,6-dichlorophenyl)-5-((4-((2-methoxyethyl)carbamoyl)phenyl)amino)-2H-1,2,3-triazole-4-carboxamide; 2-(2,6-dichlorophenyl)-5-((4-(3-hydroxyazetidine-1-carbonyl)phenyl)amino)-2H-1,2,3-triazole-4-carboxamide; 2-(2,6-dichlorophenyl)-5-((4-(4-isopropyl-4H-1,2,4-triazol-3-yl)phenyl)amino)-2H-1,2,3-triazole-4-carboxamide; 2-(2,6-dichlorophenyl)-5-((4-(5-ethyl-1H-1,2,3-triazol-1-yl)phenyl)amino)-2H-1,2,3-triazole-4-carboxamide; 2-(2,6-dichlorophenyl)-5-((5-(4-methylpiperazine-1-carbonyl)pyridin-2-yl)amino)-2H-1,2,3-triazole-4-carboxamide; 2-(2,6-dichlorophenyl)-5-((4-((2-fluoroethyl)carbamoyl)phenyl)amino)-2H-1,2,3-triazole-4-carboxamide; 2-(2,6-dichlorophenyl)-5-((4-(3-fluoroazetidine-1-carbonyl)phenyl)amino)-2H-1,2,3-triazole-4-carboxamide; (S)-2-(2,6-dichlorophenyl)-5-((5-(3-fluoropyrrolidine-1-carbonyl)pyridin-2-yl)amino)-2H-1,2,3-triazole-4-carboxamide; 2-(2,6-dichlorophenyl)-5-((4-(5-methyl-1H-1,2,3-triazol-1-yl)phenyl)amino)-2H-1,2,3-triazole-4-carboxamide; 2-(2,6-dichlorophenyl)-5-((5-(3,3,4,4-tetrafluoropyrrolidine-1-carbonyl)pyridin-2-yl)amino)-2H-1,2,3-triazole-4-carboxamide; (R)-2-(2,6-dichlorophenyl)-5-((5-(3-(dimethylamino)pyrrolidine-1-carbonyl)pyridin-2-yl)amino)-2H-1,2,3-triazole-4-carboxamide; 2-(2,6-dichlorophenyl)-5-((5-(5-methyl-1H-tetrazol-1-yl)pyridin-2-yl)amino)-2H-1,2,3-triazole-4-carboxamide; 2-(2,6-dichlorophenyl)-5-((5-(3-fluoroazetidine-1-carbonyl)pyridin-2-yl)amino)-2H-1,2,3-triazole-4-carboxamide; (R)-2-(2,6-dichlorophenyl)-5-((5-(3-fluoropyrrolidine-1-carbonyl)pyridin-2-yl)amino)-2H-1,2,3-triazole-4-carboxamide; 2-(2,6-dichlorophenyl)-5-((5-(pyrrolidine-1-carbonyl)pyridin-2-yl)amino)-2H-1,2,3-triazole-4-carboxamide; 2-(2,6-dichloro-3-fluorophenyl)-5-((4-(morpholine-4-carbonyl)phenyl)amino)-2H-1,2,3-triazole-4-carboxamide; 2-(2,6-dichlorophenyl)-5-((6-(5-methyl-[1,2,4]triazolo[4,3-a]pyridin-3-yl)pyridin-3-yl)amino)-2H-1,2,3-triazole-4-carboxamide; 2-(2,6-dichlorophenyl)-5-((5-(morpholine-4-carbonyl)pyridin-2-yl)amino)-2H-1,2,3-triazole-4-carboxamide; 2-(2,6-dichlorophenyl)-5-((5-(5-methyl-1H-1,2,3-triazol-1-yl)pyridin-2-yl)amino)-2H-1,2,3-triazole-4-carboxamide; 2-(2,6-dichlorophenyl)-5-((5-(1-ethyl-1H-1,2,4-triazol-5-yl)pyridin-2-yl)amino)-2H-1,2,3-triazole-4-carboxamide; 2-(2,6-dichlorophenyl)-5-((5-morpholinopyridin-2-yl)amino)-2H-1,2,3-triazole-4-carboxamide; 2-(2,6-dichlorophenyl)-5-((5-(1-methyl-4-(trifluoromethyl)-1H-imidazol-2-yl)pyridin-2-yl)amino)-2H-1,2,3-triazole-4-carboxamide; (R)-2-(2,6-dichloro-3-fluorophenyl)-5-((4-(3-fluoropyrrolidine-1-carbonyl)phenyl)amino)-2H-1,2,3-triazole-4-carboxamide; (R)-2-(2,6-dichloro-4-fluorophenyl)-5-((4-(3-fluoropyrrolidine-1-carbonyl)phenyl)amino)-2H-1,2,3-triazole-4-carboxamide; (R)-2-(2-chloro-3,6-difluorophenyl)-5-((4-(3-fluoropyrrolidine-1-carbonyl)phenyl)amino)-2H-1,2,3-triazole-4-carboxamide; (R)-2-(2,6-dichloro-4-cyanophenyl)-5-((4-(3-fluoropyrrolidine-1-carbonyl)phenyl)amino)-2H-1,2,3-triazole-4-carboxamide; 2-(2,6-dichlorophenyl)-5-((5-(4-(dimethylamino)piperidine-1-carbonyl)pyridin-2-yl)amino)-2H-1,2,3-triazole-4-carboxamide; 2-(2,6-dichlorophenyl)-5-((5-(4-methyl-1,4-diazepane-1-carbonyl)pyridin-2-yl)amino)-2H-1,2,3-triazole-4-carboxamide; 2-(2,6-dichlorophenyl)-5-((6-(2-morpholino-2-oxoethyl)pyridin-3-yl)amino)-2H-1,2,3-triazole-4-carboxamide; 2-(2,6-dichlorophenyl)-5-((6-(2-(3-fluoroazetidin-1-yl)-2-oxoethyl)pyridin-3-yl)amino)-2H-1,2,3-triazole-4-carboxamide; 5-((5-(3-cyanoazetidine-1-carbonyl)pyridin-2-yl)amino)-2-(2,6-dichlorophenyl)-2H-1,2,3-triazole-4-carboxamide; 2-(2,6-dichlorophenyl)-5-((5-(3-hydroxyazetidine-1-carbonyl)pyridin-2-yl)amino)-2H-1,2,3-triazole-4-carboxamide; 2-(2,6-dichlorophenyl)-5-((4-(5-(trifluoromethyl)-1H-1,2,3-triazol-1-yl)phenyl)amino)-2H-1,2,3-triazole-4-carboxamide; 2-(2,6-dichlorophenyl)-5-((5-(5-methyl-1H-1,2,4-triazol-1-yl)pyridin-2-yl)amino)-2H-1,2,3-triazole-4-carboxamide; 2-(2,6-dichlorophenyl)-5-((5-(5-(trifluoromethyl)-1H-1,2,3-triazol-1-yl)pyridin-2-yl)amino)-2H-1,2,3-triazole-4-carboxamide; 2-(2,6-dichlorophenyl)-5-((5-(1-methyl-1H-tetrazol-5-yl)pyridin-2-yl)amino)-2H-1,2,3-triazole-4-carboxamide; 2-(2,6-dichlorophenyl)-5-((6-(5-(trifluoromethyl)-1H-1,2,3-triazol-1-yl)pyridin-3-yl)amino)-2H-1,2,3-triazole-4-carboxamide; 2-(2,6-dichlorophenyl)-5-((5-(1,1-dioxidothiomorpholine-4-carbonyl)pyridin-2-yl)amino)-2H-1,2,3-triazole-4-carboxamide; 2-(2,6-dichlorophenyl)-5-((6-(5-methyl-[1,2,4]triazolo[4,3-a]pyrazin-3-yl)pyridin-3-yl)amino)-2H-1,2,3-triazole-4-carboxamide; (R)-2-(2-chloro-6-cyanophenyl)-5-((4-(3-fluoropyrrolidine-1-carbonyl)phenyl)amino)-2H-1,2,3-triazole-4-carboxamide; 2-(2,6-dichlorophenyl)-5-((4-(1,1-dioxidothiomorpholine-4-carbonyl)phenyl)amino)-2H-1,2,3-triazole-4-carboxamide; (R)-2-(2-cyano-6-fluorophenyl)-5-((4-(3-fluoropyrrolidine-1-carbonyl)phenyl)amino)-2H-1,2,3-triazole-4-carboxamide; 2-(2,6-dichlorophenyl)-5-((5-(3,3-dimethylazetidine-1-carbonyl)pyridin-2-yl)amino)-2H-1,2,3-triazole-4-carboxamide; 2-(2,6-dichlorophenyl)-5-((4-(3,3-dimethylazetidine-1-carbonyl)phenyl)amino)-2H-1,2,3-triazole-4-carboxamide; (R)-2-(6-chloro-2,3-difluorophenyl)-5-((4-(3-fluoropyrrolidine-1-carbonyl)phenyl)amino)-2H-1,2,3-triazole-4-carboxamide; 2-(2,6-dichlorophenyl)-5-((1-(2-morpholino-2-oxoethyl)-1H-pyrazol-4-yl)amino)-2H-1,2,3-triazole-4-carboxamide; 2-(2,6-dichlorophenyl)-5-((4-(1-imino-1-oxidothiomorpholine-4-carbonyl)phenyl)amino)-2H-1,2,3-triazole-4-carboxamide; 2-(2,6-dichlorophenyl)-5-((4-(thiomorpholine-4-carbonyl)phenyl)amino)-2H-1,2,3-triazole-4-carboxamide; 2-(2,6-dichlorophenyl)-5-((4-(1-(methylimino)-1-oxidothiomorpholine-4-carbonyl)phenyl)amino)-2H-1,2,3-triazole-4-carboxamide; 2-(2,6-dichlorophenyl)-5-((1-(methylsulfonyl)-1H-pyrazol-4-yl)amino)-2H-1,2,3-triazole-4-carboxamide; 2-(2,6-dichlorophenyl)-5-((1-(ethylsulfonyl)-1H-pyrazol-4-yl)amino)-2H-1,2,3-triazole-4-carboxamide; 2-(2,6-dichlorophenyl)-5-((1-(2-(1,1-dioxidothiomorpholino)-2-oxoethyl)-1H-pyrazol-4-yl)amino)-2H-1,2,3-triazole-4-carboxamide; 2-(2,6-dichloro-4-fluorophenyl)-5-((4-(1,1-dioxidothiomorpholine-4-carbonyl)phenyl)amino)-2H-1,2,3-triazole-4-carboxamide; 2-(2,6-dichloro-4-methoxyphenyl)-5-((4-(thiomorpholine-4-carbonyl)phenyl)amino)-2H-1,2,3-triazole-4-carboxamide; and 2-(2,6-dichloro-4-fluorophenyl)-5-((4-(thiomorpholine-4-carbonyl)phenyl)amino)-2H-1,2,3-triazole-4-carboxamide.

41. The method of claim 21, wherein the kinase is LRRK2, NUAK1, JAK1, JAK2, or TYK2.
